(19) United States
(12) Patent Application Publication
(10) Pub. No.: US 2017/0333237 A1
(43) Pub. Date: Nov. 23, 2017

(54) VESSEL ACCESS CATHETER

(71) Applicant: Walzman Innovations, LLC, Boynton Beach, FL (US)

(72) Inventor: Daniel Walzman, Bergenfield, NJ (US)

(21) Appl. No.: 15/482,436

(22) Filed: Apr. 7, 2017

Related U.S. Application Data
(63) Continuation-in-part of application No. 15/250,693, filed on Aug. 29, 2016.
Application No. 15/250,693 is a continuation-in-part of application No. 15/158,341, filed on May 18, 2016.

(51) Int. Cl. A61F 2/958; A61M 25/01; A61F 2/966; A61M 25/00

(52) U.S. Cl. CPC A61F 2/958; A61M 25/0041; A61M 25/0068; A61M 25/0152; A61F 2/966; A61M 25/0133; A61M 2210/127; A61M 25/01

(57) ABSTRACT

The described invention provides endovascular devices that share the following general features: a working segment, a support segment, a working lumen, a support lumen, a side hole, and an angled extension. A described endovascular device includes an outer support catheter and an inner catheter with a side hole optionally containing an angled extension disposed at least partially within the lumen defined by the outer support catheter. The inner catheter can be connected to sets of grips on the outer catheter and the inner catheter, which are used by the operator to rotate one catheter relative to the other so as to position the side hole or the angled extension of the inner catheter in the targeted vessel. A radiopaque marker or intravascular ultrasound can be used to identify the position of the distal side hole in vivo. In some devices, the distal segment beyond the side hole (the support segment) for each of the described devices is effective to provide stability to each endovascular device, to provide strength to the endovascular device, to provide support for the endovascular device, to facilitate placement of the endovascular device, to anchor the endovascular device within a blood vessel, to reduce kickback of the endovascular device, or a combination thereof. The endovascular devices described can include additional support elements, for example, a balloon, a stent, a wire, or a combination thereof. The described selectively inflatable balloon is of two dimensions, a diameter and a length, sized to fit within the diameter of a vessel, for positioning the endovascular device. It may be disposed in a circumferential array of selectively inflatable balloons, as a single distal inflatable balloon or both. The circumferential array of selectively inflatable balloons is effective to position the endovascular device within a target blood vessel. The single distal balloon is effective to anchor the endovascular device within a target blood vessel. Each inflatable balloon can be selectively filled with a fluid, e.g., sterile water and saline.

### CROSS-REFERENCE TO RELATED APPLICATIONS

This application is a continuation-in-part (CIP) of U.S. patent application Ser. No. 15/250,693, filed Aug. 29, 2016 and U.S. patent application Ser. No. 15/158,341, filed May 18, 2016. The content of each of these applications is incorporated by reference herein in its entirety.

### FIELD OF THE INVENTION

The described invention relates generally to endovascular devices.

### BACKGROUND OF THE INVENTION

### Blood Vessel Structure and Function

Blood vessels are dynamic structures that constrict, relax, pulsate, and proliferate. Within the body, blood vessels form a closed delivery system that begins and ends at the heart. There are three major types of blood vessels: (i) arteries; (ii) capillaries and (iii) veins. As the heart contracts, it forces blood into the large arteries leaving the ventricles. Blood then moves into smaller arteries successively, until finally reaching the smallest branches, the arterioles, which feed into the capillary beds of organs and tissues. Blood drains from the capillaries into venules, the smallest veins, and then into larger veins that merge and ultimately empty into the heart.

Arteries carry blood away from the heart and “branch” as they form smaller and smaller divisions. In contrast, veins carry blood toward the heart and “merge” into larger and larger vessels approaching the heart. In the systemic circulation, arteries carry oxygenated blood and veins carry oxygen-poor blood. In the pulmonary circulation, the opposite is true. The arteries (still defined as the vessels leading away from the heart), carry oxygen-poor blood to the lungs, and the veins carry oxygen-rich blood from the lungs to the heart.

The only blood vessels that have intimate contact with tissue cells in the human body are capillaries. In this way, capillaries help serve cellular needs. Exchanges between the blood and tissue cells occur primarily through the thin capillary walls.

The walls of most blood vessels (the exception being the smallest vessels, e.g., venules), have three layers, or tunics, that surround a central blood-containing space called the vessel segment.

The innermost tunic (layer) is the tunica intima. The tunica intima contains the endothelium, the simple squamous epithelium that lines the segment of all vessels. The endothelium is continuous with the endocardial lining of the heart, and its flat cells fit closely together, forming a slippery surface that minimizes friction so blood moves smoothly through the segment. In vessels larger than 1 mm in diameter, a sub-endothelial layer, consisting of a basement membrane and loose connective tissue, supports the endothelium.

The middle tunic (layer), the tunica media, is mostly circularly arranged smooth muscle cells and sheets of elastin. The activity of the smooth muscle is regulated by sympathetic vasomotor nerve fibers of the autonomic nervous system. Depending on the body's needs at any given time, regulation causes either vasoconstriction (segment diameter decreases) or vasodilation (segment diameter increases). The activities of the tunica media are critical in regulating the circulatory system because small changes in vessel diameter greatly influence blood flow and blood pressure. Generally, the tunica media is the bulkiest layer in arteries, which bear the chief responsibility for maintaining blood pressure and proper circulation.

The outer layer of a blood vessel wall, the tunica externa, is primarily composed of collagen fibers that protect the vessel, reinforce the vessel, and anchor the vessel to surrounding structures. The tunica externa contains nerve fibers, lymphatic vessels, and elastic fibers (e.g., in large veins). In large vessels, the tunica externa contains a structure known as the vasa vasorum, which literally means “vessels of vessels”. The vasa vasorum nourishes external tissues of the blood vessel wall. Interior layers of blood vessels receive nutrients directly from blood in the segment (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

Cerebral Arteries

FIGS. 8 and 9 show schematic illustrations of the brain's blood vessels. Each cerebral hemisphere is supplied by an internal carotid artery, which arises from a common carotid artery beneath the angle of the jaw, enters the cranium through the carotid foramen, traverses the cavernosus sinus (giving off the ophthalmic artery), penetrates the dura and divides into the anterior and middle cerebral arteries. The large surface branches of the anterior cerebral artery supply the cortex and white matter of the inferior frontal lobe, the medial surface of the frontal and parietal lobes and the anterior corpus callosum. Smaller penetrating branches supply the deeper cerebrum and diencephalon, including limbic structures, the head of the caudate, and the anterior limb of the internal capsule. The large surface branches of the middle cerebral artery supply most of the cortex and white matter of the hemisphere's convexity, including the frontal, parietal, temporal and occipital lobes, and the insula. Smaller penetrating branches supply the deep white matter and diencephalic structures such as the posterior limb of the internal capsule, the putamen, the outer globus pallidus, and the body of the caudate. After the internal carotid artery emerges from the cavernous sinus, it also gives off the anterior choroidal artery, which supplies the anterior hippocampus and, at a caudal level, the posterior limb of the internal capsule. Each vertebral artery arises from a subclavian artery, enters the cranium through the foramen magnum, and gives off an anterior spinal artery and a posterior inferior cerebellar artery. The vertebral arteries join at the junction of the pons and the medulla to form the basilar artery, which at the level of the pons gives off the anterior inferior cerebellar artery and the internal auditory artery, and, at the midbrain, the superior cerebellar artery. The basilar artery then divides into the two posterior cerebral arteries. The large surface branches of the posterior cerebral arteries supply the inferior temporal and medial occipital lobes and the posterior corpus callosum; the smaller penetrating branches of these arteries supply diencephalic structures, including the thalamus and the subthalamic nuclei, as well as part of the midbrain (see Principles of Neural Sciences, 2d Ed., Eric R. Kandel and James H. Schwartz, Elsevier Science Publishing Co., Inc., New York, pp. 854-56 (1985)).

Interconnections between blood vessels (anastomoses) protect the brain when part of its vascular supply is compromised. Anastomoses are interconnections between blood vessels that protect the brain when part of its vascular supply is compromised. At the circle of Willis, the two anterior cerebral arteries are connected by the anterior communicating artery and the posterior cerebral arteries are connected to the internal carotid arteries by the posterior communicating arteries. Other important anastomoses include connections between the ophthalmic artery and branches of the external carotid artery through the orbit, and connections at the brain surface between branches of the middle, anterior, and posterior cerebral arteries (Principles of Neural Sciences, 2d Ed., Eric R. Kandel and James H. Schwartz, Elsevier Science Publishing Co., Inc., New York, pp. 854-56 (1985)).

Femoral Artery

The femoral artery is the main artery that provides oxygenated blood to the tissues of the leg. It passes through the deep tissues of the femoral (or thigh) region of the leg parallel to the femur.

The common femoral artery is the largest artery found in the femoral (thigh) region of the body. It begins as a continuation of the external iliac artery at the inguinal ligament which serves as the dividing line between the pelvis and the leg. From the inguinal ligament, the femoral artery follows the medial side of the head and neck of the femur inferiorly and laterally before splitting into the deep femoral artery and the superficial femoral artery.

The superficial femoral artery flexes to follow the femur inferiorly and medially. At its distal end, it flexes again and descends posterior to the femur before forming the popliteal artery of the posterior knee and continuing on into the lower leg and foot. Several smaller arteries branch off from the superficial femoral artery to provide blood to the skin and superficial muscles of the thigh.

The deep femoral artery follows the same path as the superficial branch, but follows a deeper path through the tissues of the thigh, closer to the femur. It branches off into the lateral and medial circumflex arteries and the perforating arteries that wrap around the femur and deliver blood to the femur and deep muscles of the thigh. Unlike the superficial femoral artery, none of the branches of the deep femoral artery continue into the lower leg or foot.

Like most blood vessels, the femoral artery is made of several distinct tissue layers that help it to deliver blood to the tissues of the leg. The innermost layer, known as the endothelium or tunica intima, is made of thin, simple squamous epithelium that holds the blood inside the hollow segment of the blood vessel and prevents platelets from sticking to the surface and forming blood clots. Surrounding the tunica intima is a thicker middle layer of connective tissues known as the tunica media. The tunica media contains many elastic and collagen fibers that give the femoral artery its strength and elasticity to withstand the force of blood pressure inside the vessel. Visceral muscle in the tunica media may contract or relax to help regulate the amount of blood flow. Finally, the tunica externa is the outermost layer of the femoral artery that contains many collagen fibers to reinforce the artery and anchor it to the surrounding tissues so that it remains stationary.

The femoral artery is classified as an elastic artery, meaning that it contains many elastic fibers that allow it to stretch in response to blood pressure. Every contraction of the heart causes a sudden increase in the blood pressure in the femoral artery, and the artery wall expands to accommodate the blood. This property allows the femoral artery to be used to detect a person's pulse through the skin (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

Use of the Femoral Artery for Endovascular Procedures

Endovascular diagnostic and therapeutic procedures are generally performed through the femoral artery. Some of the reasons for this generalized approach include its location, easy approach for puncture and hemostasis, low rate of complications, technical ease, wide applicability and relative patient comfort (Alvarez-Tostado J. A. et al. Journal of Vascular Surgery 2009; 49(2): 378-385). Femoral puncture also allows access to virtually all of the arterial territories and affords favorable ergonomics for the operator in most instances (Alvarez-Tostado J. A. et al. Journal of Vascular Surgery 2009; 49(2): 378-385).

Brachial Artery

The brachial artery is a major blood vessel located in the upper arm and is the main supplier of blood to the arm and hand. It continues from the axillary artery at the shoulder and travels down the underside of the arm. Along with the medial cubital vein and bicep tendon, it forms the cubital fossa, a triangular pit on the inside of the elbow. Below the cubital fossa, the brachial artery divides into two arteries running down the forearm: the ulnar and the radial; the two main branches of the brachial artery. Other branches of the brachial artery include the inferior ulnar collateral, profunda brachii, and superior ulnar arteries (See, e.g., The Cardiovascular System at a Glance, 4^{th} Edition, Philip I. Aaronson, Jeremy P. T. Ward, Michelle J. Connolly, November 2012, © 2012, Wiley-Blackwell, Hoboken, N.J.).

### Use of the Brachial Artery for Endovascular Procedures

Brachial artery access is a critical component of complex endovascular procedures, especially in instances where femoral access is difficult or contraindicated, such as the absence of palpable femoral pulses, severe common femoral occlusive disease, recent femoral intervention or surgery or femoral aneurysms/pseudoaneurysms. It is a straightforward procedure with a high success rate for percutaneous cannulation (Alvarez-Tostado J. A. et al. Journal of Vascular Surgery 2009; 49(2): 378-385). However, there is a general reluctance to puncture the right brachial artery due to the need to navigate through the innominate artery and arch and due to the risk for complications such as direct nerve trauma and ischemic occlusion resulting in long-term disability (Alvarez-Tostado J. A. et al. Journal of Vascular Surgery 2009; 49(2): 378-385; Cousins T. R. and O'Donnell J. M. AANA Journal 2004; 72(4): 267-271).

### Endovascular Intervention

The current standard for therapeutic recanalization and reperfusion in vascular disease and acute stroke is to perform endovascular interventions via a transfemoral approach, meaning, starting a catheter in the femoral artery at the groin, proceeding through the aorta and carotid artery to the affected blood vessel. All existing devices are designed to be used from this starting point and surgeons are most familiar and comfortable with this route.

Mechanical Endovascular Intervention in Coronary Artery Disease (CAD)

Percutaneous Coronary Intervention (PCI)

Percutaneous coronary intervention (PCI) is a nonsurgical method for coronary artery revascularization. PCI methods include balloon angioplasty, coronary stenting, atherectomy (devices that ablate plaque), thrombectomy (devices that remove clots from blood vessels) and embolic protection (devices that capture and remove embolic debris).

Balloon Angioplasty

Balloon angioplasty involves advancing a balloon-tipped catheter to an area of coronary narrowing, inflating the balloon, and then removing the catheter after deflation. Balloon angioplasty can reduce the severity of coronary stenosis, improve coronary flow, and diminish or eliminate objective and subjective manifestations of ischemia (Losordo D. W. et al. Circulation 1992 December. 86(6):1845-58). The mechanism of balloon angioplasty action involves three events: plaque fracture, compression of the plaque, and stretching of the vessel wall. These lead to expansion of the external elastic lumina and axial plaque redistribution along the length of the vessel (Losordo D. W. et al. Circulation 1992 December. 86(6):1845-58).

Coronary Stenting

Coronary stents are metallic scaffolds that are deployed within a diseased coronary artery segment to maintain wide luminal patency. They were devised as permanent endoluminal prostheses that could seal dissections, create a predictably large initial segment, and prevent early recoil and late vascular remodeling (Krajcer Z. and Howell M. H. Tex Heart Inst J. 2000; 27(4): 369-385).

Drug-eluting stents (DESs) elute medication to reduce restenosis (the recurrence of abnormal narrowing of a blood vessel) within the stents. Local release of rapamycin and its derivatives or of paclitaxel from a polymer matrix on the stent during the 30 days after implantation has been shown to reduce inflammation and smooth muscle cell proliferation within the stent, decreasing in-stent late loss of luminal diameter from the usual 1 mm to as little as 0.2 mm (Stone G. W. et al. N Engl J Med. 2007 Mar. 8. 356(10):998-1008). This dramatically lowers the restenosis rate after initial stent implantation or after secondary implantation of a DES for an in-stent restenosis (Stone G. W. et al. N Engl J Med. 2007 Mar. 8. 356(10):998-1008).

Coronary stents are used in about 90% of interventional procedures. Stent-assisted coronary intervention has replaced coronary artery bypass graft (CABG) as the most common revascularization procedure in patients with coronary artery disease (CAD) and is used in patients with multi-vessel disease and complex coronary anatomy (Kalyanasundaram A. et al. Medscape Dec. 16, 2014; article 164682; emedicine.medscape.com/article/164682-overview#a3).

Atherectomy

The directional coronary atherectomy (DCA) catheter was first used in human peripheral vessels in 1985 and in coronary arteries in 1986. In this procedure, a low-pressure positioning balloon presses a windowed steel housing against a lesion; any plaque that protrudes into the window is shaved from the lesion by a spinning cup-shaped cutter and trapped in the device's nose cone (Hinohara T. et al. Circulation 1990 March. 81(3 Suppl):IV79-91).

Rotational atherectomy uses a high-speed mechanical rotational stainless steel burr with a diamond chip—embedded surface. The burr is attached to a hollow flexible drive shaft that permits it to be advanced over a steerable guide wire with a platinum coil tip. The drive shaft is encased within a Teflon® sheath through which a flush solution is pumped to lubricate and cool the drive shaft and burr. A compressed air turbine rotates the drive shaft at 140,000-200,000 rpm during advancement across a lesion (Hinohara T. et al. Circulation 1990 March. 81(3 Suppl):IV79-91).

Laser Ablation

In laser ablation, an intense light beam travels via optical fibers within a catheter and enters the coronary segment. After the target lesion is crossed with the guide wire, the laser catheter is advanced to the proximal end of the lesion. Blood and contrast medium are removed from the target vessel by flushing with saline before activating the laser (Kalyanasundaram A. et al. Medscape Dec. 16, 2014; article 164682; emedicine.medscape.com/article/164682-overview#a3).

Mechanical Thrombectomy

Intracoronary thrombi can be treated with mechanical thrombectomy devices. These include rheolytic, suction and ultrasonic thrombectomy devices.

In rheolytic thrombectomy, high-speed water jets create suction via the Bernoulli-Venturi effect. The jets exit orifices near the catheter tip and spray back into the mouth of the catheter, creating a low-pressure region and intense suction. This suction pulls surrounding blood, thrombus, and saline into the tip opening and propels particles proximally through the catheter segment and out of the body (Kalyanasundaram A. et al. Medscape Dec. 16, 2014; article 164682; medicine.medscape.com/article/164682-overview#a 3).

The catheters used for suction thrombectomy act via manual aspiration. These catheters are advanced over a wire to the intracoronary thrombus then passed through the thrombus while suction is applied to a hole in the catheter tip. Large intact thrombus fragments can be removed by means of this technique (Kalyanasundaram A. et al. Medscape Dec. 16, 2014; article 164682; emedicine.medscape.com/article/164682-overview#a3).

Ultrasonic thrombectomy involves the use of ultrasonic vibration to induce cavitation that can fragment a thrombus into smaller components (Choi S.W. et al. J. Intery Cardiol. 2006 Feb. 19(1): 87-92).

Embolization Protection

Embolization (the passage of an embolus (blood clot) within the blood stream) can be caused by the manipulation of guidewires, balloons, and stents across complex atherosclerotic carotid artery lesions (Krajcer Z. and Howell M. H. Tex Heart Inst J. 2000; 27(4): 369-385). Several devices have been developed to trap such embolic material and remove it from the circulation.

The PercuSurge Guardwire is a device that consists of a 0.014- or 0.018-inch angioplasty guidewire constructed of a hollow nitinol hypotube. Incorporated into the distal wire segment is an inflatable balloon capable of occluding vessel flow. The proximal end of the wire incorporates a Microseal™ that allows inflation and deflation of the distal occlusion balloon. When the Microseal adapter is detached, the occlusion balloon remains inflated, at which time angioplasty and stenting are performed. An aspiration catheter can be advanced over the wire into the vessel, and manual suction is applied to retrieve particulate debris (Krajcer Z. and Howell M. H. Tex Heart Inst J. 2000; 27(4): 369-385).

The Medicorp device consists of a protection balloon and a dilation balloon that can be used over a 0.014-inch coronary guidewire. Occlusion above the lesion and below the lesion creates a dilation zone without a flow, which is aspirated and cleared of atherosclerotic debris (Krajcer Z. and Howell M. H. Tex Heart Inst J. 2000; 27(4): 369-385).

Endovascular Treatment of Abdominal Aortic Aneurysms (AAA)

Two endoluminal AAA exclusion stent graft systems have received FDA approval: (i) the Ancure™ Endograft System (Guidant/EVT; Menlo Park, Calif); and (ii) the AneuRx™ device (Medtronic AVE; Santa Rosa, Calif) (Krajcer Z. and Howell M. H. Tex Heart Inst J. 2000; 27(4): 369-385). Both are over-the-wire systems that require bilateral femoral artery access.

The Ancure™ stent graft is an unsupported, single piece of woven Dacron® fabric. The graft is bifurcated and has no intra-graft junctions. The main device is delivered through a 24-Fr introducer sheath; a 12-Fr sheath is required to facilitate the deployment of the contralateral iliac limb. The graft is attached via a series of hooks that are located at the proximal aortic end and at both iliac ends. The hooks are seated transmurally (passing through the vessel wall) in the aorta and the iliac arteries, initially by minimal radial force, and then affixed by low-pressure balloon dilation. Radiopaque markers are located on the body of the graft for correct alignment and positioning (Krajcer Z. and Howell M.H. Tex Heart Inst J. 2000; 27(4): 369-385).

The AneuRx™ device is a modular 2-piece system composed of a main bifurcation segment and a contralateral iliac limb. The graft is made of thin-walled woven polyester that is fully supported by a self-expanding nitinol exoskeleton. Attachment is accomplished by radial force at the attachment sites, which causes a frictional seal. The main bifurcated body is delivered through a 21-Fr sheath, and the contralateral limb requires a 16-Fr sheath. The body of the graft has radiopaque markers that facilitate correct alignment and positioning (Krajcer Z. and Howell M. H. Tex Heart Inst J. 2000; 27(4): 369-385).

### Mechanical Endovascular Neurointervention

Mechanical Thrombectomy

Mechanical thrombectomy (excision of a clot from a blood vessel) devices remove occluding thrombi (blood clots) from the target vessel by a catheter. Subgroups include (1) suction thrombectomy devices that remove occlusions from the cerebral vessels by aspiration (Proximal Thrombectomy) and (2) clot removal devices that physically seize cerebral thrombi and drag them out of the cerebral vessels (Distal Thrombectomy) (Gralla J. et al. Stroke 2006; 37: 3019-24; Brekenfeld C. et al. Stroke 2008; 39: 1213-9).

Proximal Endovascular Thrombectomy

Manual suction thrombectomy is performed by moving forward an aspiration catheter at the proximal surface of the thrombus (Singh P. et al. J Neurosci Rural Pract. 2013 July-September; 4(3): 298-303). Manual aspiration is then carried out and the aspiration catheter is taken back under continuous negative pressure. The Penumbra System (Penumbra, Almeda, Calif. USA) is a variation of the manual proximal aspiration method which comprises a dedicated reperfusion catheter attached to a pumping system applying constant aspiration. A second retriever device is similar to a stent and is utilized to take out the resistant clot (Singh P. et al. J Neurosci Rural Pract. 2013 July-September; 4(3): 298-303). The time window for neuroradiological intervention is 8 hours after stroke onset in patients not eligible for intravenous thrombolysis or in patients where intravenous thrombolysis was unsuccessful (Singh P. et al. J Neurosci Rural Pract. 2013 July-September; 4(3): 298-303).

The Penumbra System™ has been examined in a number of clinical trials. The Penumbra Pivotal Stroke Trial was a prospective, single-arm, multicenter study that recruited 125 stroke patients (mean NIHSS 18) within 8 hours of symptom onset and was successful in 81.6% of treated vessels (Penumbra Pivotal Stroke Trial Investigators: The Penumbra pivotal stroke trial: Safety and effectiveness of a new generation of mechanical devices for clot removal in intracranial large vessel occlusive disease. Stroke 2009; 40: 2761-8). However, a good clinical outcome at 90 days was attained in only 25% of patients and in 29% of patients with successful recanalization (the process of restoring flow to or reuniting an interrupted channel such as a blood vessel) of the target vessel (Penumbra Pivotal Stroke Trial Investigators: The penumbra pivotal stroke trial: Safety and effectiveness of a new generation of mechanical devices for clot removal in intracranial large vessel occlusive disease. Stroke. 2009; 40: 2761-8). Poor clinical results occurred despite comparatively better recanalization rates as evidenced by a mortality rate of 32.8% and the occurrence of symptomatic intracerebral hemorrhage (ICH) in 11.2% (Penumbra Pivotal Stroke Trial Investigators: The penumbra pivotal stroke trial: Safety and effectiveness of a new generation of mechanical devices for clot removal in intracranial large vessel occlusive disease. Stroke. 2009; 40: 2761-8).

Distal Endovascular Thrombectomy

Distal thrombectomy is a technically difficult procedure (Singh P. et al. J Neurosci Rural Pract. 2013 July-September; 4(3): 298-303). A number of clinical studies have been carried out using the MERCI (Mechanical Embolus Removal in Cerebral Ischemia) Retriever® device (Concentric Medical, Mountain View, USA), which was the earliest distal thrombectomy device approved by the United States Food and Drug Administration (FDA) (Singh P. et al. J Neurosci Rural Pract. 2013 July-September; 4(3): 298-303). In the initial stage of the procedure, the occlusion site must be traversed with a microcatheter so as to deploy the device beyond the thrombus. The MERCI Retriever® device is pulled back into the thrombus and positioned within the clot. Next, the MERCI Retriever® and the trapped clot are withdrawn, initially into the positioning catheter and then out of the patient's body (Singh P. et al. J Neurosci Rural Pract. 2013 July-September; 4(3): 298-303). Proximal balloon occlusion by means of a balloon guide catheter and aspiration during retrieval of the Merci device is done for the majority of cases in order to prevent thromboembolic complications (Nogueira R. G. et al. Am J Neuroradiol. 2009; 30: 649-61; Nogueira R. G. et al. Am J Neuroradiol. 2009; 30: 859-7). During in vivo experimental studies, the distal technique was shown to be more efficient as compared to proximal manual aspiration (Gralla J. et al. Stroke 2006; 37: 3019-24).

The MERCI Retriever® clinical trial was a 25-site, uncontrolled, technical efficacy trial (Smith W. S. et al. Stroke 2005; 36: 1432-8). The trial incorporated 151 patients with occlusion of the internal carotid artery or vertebral and basilar arteries, who did not qualify for intra-arterial therapy (IAT) within 8 hours of symptom onset (Smith W. S. et al. Stroke 2005; 36: 1432-8). Successful recanalization was accomplished in 46%, with excellent clinical outcome in 27.7% of patients (Smith W. S. et al. Stroke 2005; 36: 1432-1438). Successful recanalization was linked with distinctly better clinical outcomes. Average procedure time was 2.1 hours, with clinically noteworthy procedural complications occurring in 7.1% and a rate of symptomatic intracranial hemorrhage (ICH) occurring in 7.8% of patients (Smith W. S. et al. Stroke 2005; 36: 1432-1438). Despite good clinical outcome, limitations of this device include operator learning curve, the need to traverse the occluded artery to deploy the device distal to the occlusion, the duration required to perform multiple passes with device, clot fragmentation and passage of an embolus within the bloodstream (Meyers P. M. et al. Circulation 2011; 123: 2591-2601).

Self-Expanding Stents

Until recently, intracranial stenting was restricted to off-label use of balloon-mounted stents intended for cardiac circulation (Singh P. et al. J Neurosci Rural Pract. 2013 July-September; 4(3): 298-303). These stents are not ideal for treating intracranial disease due to their rigidity which makes navigation in the convoluted intracranial vessels difficult (Singh P. et al. J Neurosci Rural Pract. 2013 July-September; 4(3): 298-303). Self-expanding intracranial stents permit stenting in acute stroke that is unmanageable with conventional treatment regimens. The clot occluding the vessel is outwardly displaced by the side of the vessel wall and becomes trapped in the interstices of a self-expanding stent (SES). Wingspan™ (Stryker), Neuroform® (Stryker, Kalamazoo, Mich.), and Cordis Enterprise™ (Cordis Neurovascular, Fremont, Calif.) self-expanding stenting systems have improved steering, cause a reduced amount of vasospasm, and cause a reduced amount of side-branch occlusions as compared to balloon-inflated stents Singh P. et al. J Neurosci Rural Pract. 2013 July-September; 4(3): 298-303). Drawbacks of this method include delayed in-stent thrombosis, the use of platelet inhibitors which may cause intracerebral hemorrhage (ICH) and perforator occlusion from relocation of the thrombus after stent placement (Samaniego E. A. et al Front Neurol. 2011; 2: 1-7; Fitzsimmons B. F. et al. Am J Neuroradiol. 2006; 27: 1132-4; Levy E. I. et al. Neurosurgery 2006; 58: 458-63; Zaidat O. O. et al. Stroke 2008; 39: 2392-5).

Retrievable Thrombectomy Stents

Retrievable thrombectomy stents are self-expandable, re-sheathable, and re-constrainable stent-like thrombectomy devices which combine the advantages of intracranial stent deployment with immediate reperfusion and subsequent retrieval with definitive clot removal from the occluded artery (Singh P. et al. J Neurosci Rural Pract. 2013 July-September; 4(3): 298-303). Removal of the device circumvents the drawbacks associated with permanent stent implantation. These include the requirement for double anti-platelet medication, which potentially adds to the risk of hemorrhagic complications and the risk of in-stent thrombosis or stenosis. The application of retrievable thrombectomy stents is analogous to that of intracranial stents. Under general anesthesia, using a transfemoral approach, a guide catheter is positioned in the proximal internal carotid artery. A guide wire is advanced coaxially over a microcatheter within the blocked intracranial vessel and navigated past the thrombus. The microcatheter is then advanced over the wire through the clot, and the guide wire is substituted for the embolectomy device Singh P. et al. J Neurosci Rural Pract. 2013 July-Sepember; 4(3): 298-303). The revascularization device is placed with the middle third of the device residing within the thrombus formation. The radial force of the stent retriever is able to create a channel by squeezing the thrombus and is able to partially restore blood flow to the distal territory in the majority of cases, producing a channel for a temporary bypass (Singh P. et al. J Neurosci Rural Pract. 2013 July-September; 4(3): 298-303). The device is usually left in place for an embedding time of up to 10 minutes, permitting entrapment of the thrombus within the stent struts. To extract the thrombus, the unfolded stent and the microcatheter are slowly dragged into the guide catheter with flow reversal by continuous aspiration with a 50-ml syringe from the guide catheter Singh P. et al. J Neurosci Rural Pract. 2013 July-September; 4(3): 298-303). The designs of these stents differ in terms of radial strength, design of the proximal and distal stent aperture, stent cell design, material and supplementary intraluminal struts (Mordasini P. et al. Am J Neuroradiol 2011; 32: 294-300; Brekenfeld C. et al. Am J Neuroradiol. 201; 2: 1269-73; Mordasini P. et al. Am J Neuroradiol. 2013; 34: 153-8). Despite the potential to diminish procedure time and to improve recanalization rates, drawbacks to using these devices remain. For example, the TREVO 2 study (Thrombectomy Revascularisation of Large Vessel Occlusions in AIS) was an open label, multi-center trial evaluating the efficacy of the Trevo Pro retriever (Stryker Neurovascular, Fremont, USA) with the Merci device in patients with large vessel ischemic stroke (Nogueira R. G. et al. Lancet 2012; 380: 1231-40). Symptomatic ICH occurred in 6.8% in the Trevo group and in 8.9% of the Merci group, with mortality rates of 33% an 24% respectively. The outcome of this trial sustains the supposition that there are unique mechanical mechanisms of action and consequently dissimilar success and efficacy rates depending on the thrombectomy approaches applied (Singh P. et al. J Neurosci Rural Pract. 2013 July-September; 4(3): 298-303).

Although mechanical endovascular neurointerventions using a transfemoral approach are the current standard for the treatment of acute stroke, it is difficult to access the left internal carotid artery via these transfemoral techniques when an aortic arch variation occurs. A similar transfemoral access problem can occur when vertebral arteries arise at an acute angle from the subclavian artery.

### Aortic Arch

Normal Anatomy

The most common aortic arch branching pattern in humans consists of three great vessels originating from the arch of the aorta. The first branch is the innominate artery (brachiocephalic artery), which branches into the right subclavian artery and the right common carotid artery. The second branch in the most common pattern is the left common carotid artery, and the last branch is the left subclavian artery (Layton K. F. Am J Neuroradiol. 2006; 27: 1541-1542) (FIG. 3).

Variant Anatomy of the Aortic Arch

Hypoplastic Ascending Aorta

Hypoplasia (underdevelopment or incomplete development) of the ascending aorta usually occurs concomitant with hypoplastic left heart syndrome (HLHS). HLHS comprises a wide spectrum of cardiac malformations, including hypoplasia or atresia (abnormal opening or failure of a structure to be tubular) of the aortic and mitral valves and hypoplasia of the left ventricle and ascending aorta. The great vessels are normally related in this congenital anomaly. HLHS has a reported prevalence of 0.2 per 1000 live births and occurs twice as often in boys as in girls. Left untreated, HLHS is lethal (Kau T. et al. Semin Intervent Radiol. 2007; 24(2): 141-152).

Coarctation of the Aorta

Coarctation of the aorta accounts for about 5 to 7% of all congenital heart disease. It is defined as a discrete stenosis in the proximal descending thoracic aorta. Only those with the most severe obstruction (e.g., aortic arch atresia or interruption) or associated cardiac defects invariably present in infancy (Jenkins N. P., Ward C. QJM. 1999; 92: 365-371). Most other cases are identified because of a murmur or hypertension found on routine examination. Age at presentation is related to the severity rather than the site of obstruction, as a result of cardiac failure or occasionally cerebrovascular accident, aortic dissection, or endocarditis (Jenkins N. P., Ward C. QJM. 1999; 92: 365-371). Aortic coarctation can be subclassified into isolated coarctation, coarctation with ventricular septal defect, and coarctation with complex intracardiac anomalies (Backer C. L. et al. Ann Thorac Surg. 2000; 69: S308-S318). An exceedingly rare congenital anomaly is coarctation of a right aortic arch (Maxey T. S. et al. J Card Surg. 2006; 21: 261-263).

Interrupted Aortic Arch

Interrupted aortic arch is defined as the loss of luminal continuity between the ascending and descending aorta and is associated with a multitude of lesions ranging from isolated ventricular septal defects to complex ones (Kau T. et al. Semin Intervent Radiol. 2007; 24(2): 141-152). An interrupted aortic arch can be subclassified into anatomical types based on the location of the interruption (Maxey T. S. et al. J Card Surg. 2006; 21: 261-263). Although results have improved, repair of this abnormality is associated with a significant mortality and morbidity (Tchervenkov C. I. et al. Semin Thorac Cardiovasc Surg Pediatr Card Surg Annu. 2005: 92-102).

Patent Ductus Arteriosus

A ductus arteriosus Botalli permits blood flow between the aorta (distal to the left subclavian artery) and the pulmonary artery. In a full-term infant, the ductus usually closes within the first 2 days of life. Persistent patency beyond that point is generally permanent, being two to three times as common in girls as in boys. Most of the cases occur as isolated defects. Typical concomitant findings are left ventricle hypertrophy and pulmonary artery dilation. Persistent ductus arteriosus may also be associated with coarctation of the aorta, transposition of the great vessels, and ventricular septal defect (Campbell M. Br Heart J. 1968; 30:4-13).

Thyroid Ima Artery

The thyroid ima artery is a collateral vessel feeding the thyroid gland (Wolpert S. M. Radiology 1969; 92: 333-334). This vessel occurs in up to 16.9% of the population (Vasovic L. et al. Ital J Anat Embryol. 2004;109:189-197). It can be a branch of the aortic arch between the brachiocephalic and left subclavian arteries. However, more frequently it is a branch of the brachiocephalic artery. A further variant of origin is from the right common carotid artery. In the remaining cases, it may originate from the internal mammary, subclavian, or inferior thyroid arteries (Kadir S. In: Kadir S, editor. Atlas of Normal and Variant Angiographic Anatomy. Philadelphia: WB Saunders; 1991. Regional anatomy of the thoracic aorta. pp. 19-54).

Aberrant Right Subclavian or Brachiocephalic Artery

The right subclavian artery is the last branch of the aortic arch in approximately 1% of individuals (Richardson J. V. et al. Ann Thorac Surg. 1981; 31: 426-432). It courses to the right behind the esophagus in approximately 80% of these cases, between the esophagus and trachea in 15%, and anterior to the trachea or mainstem bronchus in 5% (Kadir S. In: Kadir S, editor. Atlas of Normal and Variant Angiographic Anatomy. Philadelphia: WB Saunders; 1991. Regional anatomy of the thoracic aorta. pp. 19-54).

Right Aortic Arch

Right aortic arch is an uncommon anatomical anomaly that occurs in <0.1% of the population (Cina C. S. et al. J Vasc Surg. 2004; 39: 131-139). It results from the persistence of the right fourth branchial arch (Kadir S. In: Kadir S, editor. Atlas of Normal and Variant Angiographic Anatomy. Philadelphia: WB Saunders; 1991. Regional anatomy of the thoracic aorta. pp. 19-54). The most common type is the right aortic arch with an aberrant left subclavian artery. The vessels originate in the following order: left common carotid, right common carotid, right subclavian, and left subclavian artery. This type is rarely associated with congenital heart disease. However, symptoms may arise from vascular ring formation (Son J. A. et al. J Card Surg. 999; 14: 98-102). The mirror-image type (left brachiocephalic trunk, right common carotid and subclavian arteries) is almost always associated with congenital heart disease, especially the cyanotic type (McElhinney D. B. et al. Pediatr Cardiol. 2001; 22:285-291).

Ductus Diverticulum

The aortic isthmus in adults has a variable appearance. Its configuration may show a concavity, a straightening or slight convexity, or a discrete focal bulge. The latter finding represents a ductus diverticulum, present in about 9% of individuals. Representing the most distal segment of the embryonic right arch, the ductus diverticulum is a fusiform dilation of the ventromedial portion of the proximal descending thoracic aorta. At times a prominent ductus diverticulum may resemble a traumatic pseudoaneurysm of the aortic isthmus (Goodman P. C. et al. Cardiovasc Intervent Radiol. 1982; 5: 1-4).

Double Aortic Arch

The double aortic arch is a rare anomaly caused by persistence (to varying degrees) of the fetal double aortic arch system (Kadir S. In: Kadir S, editor. Atlas of Normal and Variant Angiographic Anatomy. Philadelphia: WB Saunders; 1991. Regional anatomy of the thoracic aorta. pp. 19-54). The ascending aorta divides into two arches that pass to either side of the esophagus and trachea and reunite to form the descending aorta. Therefore, it is a form of complete vascular ring, resulting in noncardiac morbidity, but rarely associated with intracardiac defects (Alsenaidi K. et al. Pediatrics. 2006; 118: e1336-e1341). The descending aorta is usually on the left side. Most commonly, one arch is dominant, whereas the other can be of small caliber or represented by a fibrous band.

Cervical Aortic Arch

The cervical aortic arch refers to an unusually high location of the aortic arch in the low or midneck region (Kadir S. In: Kadir S, editor. Atlas of Normal and Variant Angiographic Anatomy. Philadelphia: WB Saunders; 1991. Regional anatomy of the thoracic aorta. pp. 19-54). This rare type of aortic arch anomaly is presumed to result from persistence of the third aortic arch and regression of the normal fourth arch. Abnormalities of brachiocephalic arterial branching and arch laterality are common in patients with a cervical aortic arch (McElhinney D.B. et al. Pediatr Cardiol. 2001; 22:285-291). There is no association with congenital heart disease, and the anomaly occurs most frequently in association with a right aortic arch. Most of the patients with this anomaly are asymptomatic, but symptoms of dysphagia and respiratory distress due to the compression by the vascular ring have been reported (Acikel U. et al. Angiology 1997; 48: 659-662).

Bovine Aortic Arch

A common brachiocephalic trunk (also known as the innominate artery), in which both common carotid arteries and the right subclavian artery arise from a single trunk off the arch, is the most frequent normal variant of aortic arch branching (Kadir S. In: Kadir S, editor. Atlas of Normal and Variant Angiographic Anatomy. Philadelphia: WB Saunders; 1991. Regional anatomy of the thoracic aorta. pp. 19-54) (FIG. 4). The innominate artery and the left common carotid artery have a common origin. Therefore, only 2 great vessels originate from the aortic arch (Layton K. F. et al. Am J Neuroradiol. 2006; 27: 1541-1542). Overall, this pattern of branching is seen in approximately 13% of patients (Lippert H, Pabst R. Aortic arch. In: Arterial Variations in Man: Classification and Frequency. Munich, Germany: J F Bergmann-Verlag;1985 :3-10). Although the term bovine aortic arch is ascribed to this anomaly, it is not commonly found in cattle (Layton K. F. et al. Am J Neuroradiol. 2006; 27:1541-1542).

Other Variant Branching

Variations in the sequence of branching of the major arch vessels also occur (<0.5%) (Kadir S. In: Kadir S, editor. Atlas of Normal and Variant Angiographic Anatomy. Philadelphia: WB Saunders; 1991. Regional anatomy of the thoracic aorta. pp. 19-54). For example, the left subclavian artery can be the second branch (before the left common carotid), or the internal and external carotid arteries may originate independently from the aortic arch (Nelson M. L., Sparks C.D. Clin Anat. 2001; 14: 62-65).

Variant Origin of Vertebral Arteries

Various unusual vertebral artery origins exist (Yamaki K. et al. Anat Sci Int. 2006; 81: 100-106; Koenigsberg R. A. et al. Catheter Cardiovasc Interv. 2003; 59:244-250). For example, the left vertebral artery arises from the aortic arch, with reported prevalences of 2.4 to 5.8% (Lemke A. J. et al. Am J Neuroradiol. 1999; 20: 1318-1321). The most frequent location is between the left common carotid and subclavian arteries (Kadir S. In: Kadir S, editor. Atlas of Normal and Variant Angiographic Anatomy. Philadelphia: WB Saunders; 1991. Regional anatomy of the thoracic aorta. pp. 19-54). Rarely, the proximal left vertebral artery is duplicated in which one part arises from the arch and the other from the left subclavian, or both originate from the aortic arch. Occasionally, the left vertebral artery is the last branch of the aortic arch, which is rarely true for both vertebral arteries (Goray V. B. et al. Am J Neuroradiol. 2005; 26: 93-95).

The existence of aortic and vertebral artery variations inhibits the treatment of diseases that require endovascular intervention via a transfemoral approach. For example, the acute angle at which the left common carotid artery branches from the aortic arch in the bovine arch configuration makes mechanical endovascular neurointervention difficult, especially when additional tortuosity (i.e., twists) in the aorta and/or the carotid artery are present. Currently, catheters exist that can access the origin of the left common carotid artery when arterial variations exists. However, when a wire is advanced through these catheters in order to achieve distal access to the artery head, these catheters lack adequate support which results in kickback of the advancing wire into the aortic arch. The lack of adequate support and the resulting kickback of the advancing wire make effective treatment impossible. Even when catheterization is achieved in these situations, the process of arriving at the correct combination of catheters and wires results in long treatment delays. In cases of acute stroke, long delays in obtaining access to arteries often leads to additional irreversible cell death with additional permanent neurologic injury.

A need exists for an endovascular device capable of treating diseases that require endovascular intervention in a patient suffering from a blood vessel anomaly, e.g., intracranially. The described invention provides a dual segment endovascular device capable of effectively treating such patients by providing support and thus preventing kickback of an advancing wire, resulting in distal blood vessel access, clot retrieval, embolization of an aneurysm and/or embolization of an arteriovenous malformation (AVM).

Additionally, some vessels and lesions, such as intracranial aneurysms, can originate at sharp angles and/or at indirect orientation relative to the feeding vessel. This can make stable and safe access to such a vessel and/or lesion for treatments difficult and/or dangerous. Therefore, a need exist for novel catheters that can help both position a delivery catheter for treatment accurately and/or can help stabilize such catheter and/or device in the desired position.

### SUMMARY OF THE INVENTION

According to one aspect, the described invention provides an endovascular device including: a tube defining a working lumen further comprising a support tube defining a support lumen, wherein the tube comprises: a first end; and a second end comprising an opening, wherein the opening at the second end comprises a primary opening leading to the working lumen comprising a diameter of the primary opening and a secondary opening leading to the support lumen comprising a diameter of the secondary opening, wherein the diameter of the primary opening is greater than the diameter of the secondary opening, the first branch, the working lumen and the primary opening form a working segment comprising the working lumen and the proximal portion of the support lumen; the support tube distal to the primary opening comprises a support segment, which comprises a distal segment of the support lumen and the secondary opening, and the diameter of the working segment is greater than the diameter of the support segment. According to some embodiments of the endovascular device, (a) the support segment of a single lumen device is a conduit through which a second device is advanced into a blood vessel; or (b) the working segment of a single lumen device is a conduit through which a second device is advanced into a blood vessel; or (c) the support segment or the working segment of a dual lumen device is a conduit through which a second device is advanced into a blood vessel. According to another embodiment, the second device is a diagnostic catheter, a therapeutic catheter, a therapeutic balloon, a retrievable balloon, a therapeutic stent, a retrievable stent, a flow-diverting stent, a coil, a wire, an endoluminal mesh, an endosaccular mesh, an embolic agent, another endovascular device or a combination thereof. According to another embodiment, for intracranial applications, length of the support segment of a single lumen device, ranges from 0.01% to about 20% of the length of the working segment; or, for peripheral applications, (i) length of the support segment ranges from at least 0.01% to at least 200% of the length of the working segment; or (ii) length of the support segment is about 0.05 cm to about 320 cm; or (c) length of the working segment ranges from at least 20 cm to at least 160 cm. According to another embodiment, the support segment comprises one or more inflatable balloons encircling the support segment, the working segment comprises one or more inflatable balloons encircling the working segment, or a combination thereof. According to another embodiment, (a) when a single balloon on one side of the endovascular device that comprises the side-hole is effective to deflect the device, to provide more room between the side-hole and a side-wall aneurysm, or to center the endovascular device; or (b) the two or more inflatable balloons when selectively inflated are effective to center the primary opening of the endovascular device on a base of an aneurysm in vivo; or (c) the two or more inflatable balloons when selectively inflated are effective to center the secondary opening of the endovascular device on a base of an aneurysm in vivo. According to another embodiment, the endovascular device comprises a distal inflatable balloon. According to another embodiment, (a) the selectively inflated inflatable balloons are effective to occlude blood flow; or (b) the selectively inflated inflatable balloons are effective to reverse direction of blood flow; or (c) the selectively inflated inflatable balloons are effective to position the catheter in a desired position within a blood vessel. According to another embodiment, the endovascular device further comprises a lumen positioned in association with each balloon that is adapted to fill and empty each selectively inflatable balloon. According to another embodiment, (a) the support segment comprises a second device separate from the endovascular device, wherein the second device is effective to provide added support to the endovascular device; or (b) the support segment is in form of a geometric shape selected from the group consisting of a “C” shape, a “J” shape and an “S” shape; or (c) the support segment is in form of a pre-shaped configuration; or (d) the support segment is in form of an adjustable shape; or (e) the support segment for insertion into the body of a subject is in a straight configuration and assumes a pre-formed geometric shape in situ; or (f) the support segment comprises a shape-memory polymer (SMP); or (g) the support segment comprises a shape-memory alloy (SMA). According to another embodiment, the second device separate from the endovascular device that provides added support is a stent, a retrievable stent, a balloon, a retrievable balloon, a wire or combination thereof. According to another embodiment, the second device separate from the endovascular device is adapted to be incorporated directly into the endovascular device; or is a second device adapted to be deployed through the support lumen of the endovascular device. According to another embodiment, a side-hole divides the endovascular device into a first segment and a second segment; the first segment, comprising the primary opening and the lumen, extends from the primary opening to the side hole; and forms the working segment comprising the primary opening, the lumen, and the side-hole; and the second segment, which extends from the side hole to the second opening/end; forms the support segment comprising the second opening/end and the lumen. According to another embodiment, the endovascular device is an inner catheter disposed at least partially within the working lumen of an outer support catheter, wherein (a) the inner catheter comprises a working distal side-hole that separates the working segment of the inner catheter from the distal support segment of the inner catheter; (b) the inner catheter is connected to a set of inner catheter grips at the proximal end of the endovascular device; and the outer catheter is connected to a set of outer catheter grips, wherein the inner catheter grips and outer catheter grips are adapted for rotation of one catheter relative to the other catheter; and (c) the rotation is effective to center the working distal side-hole of the inner catheter in proper alignment with a blood vessel origin, for support in accessing the blood vessel in proper alignment with a targeted lesion or other targeted anatomy, or a combination thereof. According to another embodiment, the inner catheter is a single lumen catheter; or the inner catheter is a dual lumen catheter. According to another embodiment, (a) one or more radiopaque markers can be used to identify position of the side hole; or (b) intravascular ultrasound (IVUS) can be incorporated into the endovascular device to help identify position of the side-hole relative to intraluminal anatomy including a neck of an aneurysm, a base of an aneurysm, an origin of a vessel branch, other targeted anatomy, or a combination thereof. According to another embodiment, the primary opening comprises an extension extending from the primary opening at an angle (angled extension), wherein (a) the angle of the angled extension is fixed or adjustable; or (b) the angle of the angled extension ranges from an angle of at least 1 degree to an angle of at least 359 degrees; or (c) a length of the angled extension is less than a length of the primary opening; or (d) the primary opening is adapted to accommodate extension of the angled extension through the primary opening when in a vessel. According to another embodiment, the endovascular device is an intracranial endovascular device; or a peripheral blood vessel endovascular device; or a cardiac endovascular device. According to another embodiment, the first end comprises a bifurcation, the bifurcation at the first end comprising a first branch comprising a diameter of the first branch and a length of the first branch; and a second branch comprising a diameter of the second branch and a length of the second branch. According to another embodiment, the diameter of the first branch comprising the working lumen is greater than the diameter of the second branch comprising the support lumen.

According to another aspect, the described invention provides a catheter comprising two or more inflatable balloons along a segment of its outer circumference that are adapted to be selectively inflated to position the catheter within a blood vessel.

### BRIEF DESCRIPTION OF THE DRAWINGS

FIG. 1 shows an illustration of a side view of some embodiments of a dual lumen catheter of the described invention.

FIG. 2 shows an illustration of a cross-sectional view of some embodiments of a dual lumen catheter of the described invention.

FIG. 3 shows an illustration of a side view of some embodiments of a single lumen catheter of the described invention.

FIG. 4 shows an illustration of the most common aortic arch branching pattern found in humans (from Layton K. F. et al. Am J Neuroradiol. 2006; 27: 141-1542).

FIG. 5 shows an illustration of the aortic arch branching pattern in bovine arch variation (from Layton K. F. et al. Am J Neuroradiol. 2006; 27: 141-1542).

FIG. 6 shows an illustration of a side view of some embodiments of the described invention.

FIG. 7 shows an illustration of some embodiments of the endovascular device of the described invention comprising an “S”-shape support segment inserted into an aortic arch.

FIG. 8 shows an illustrative view of the cerebral arteries.

FIG. 9 shows an illustrative view of the cerebral arteries. (from Netter FH. The CIBA Collection of Medical Illustrations: Volumes 1, Nervous System. Vol. 1. Part I. CIBA: USA. 1986. pp. 256).

FIG. 10 shows an illustrative embodiment of an endovascular device according to the described invention. The device is depicted inserted in the left middle cerebral artery (L-MCA) with the side-hole oriented to access an acutely angled branch feeder of an arteriovenous malformation (AVM).

FIG. 11 shows an illustrative embodiment of an endovascular device according to the described invention. The device is depicted inserted in the left middle cerebral artery (L-MCA) with the side-hole oriented to access an acutely angled branch feeder of an arteriovenous malformation (AVM). The device is depicted with an angled extension protruding from the side-hole.

FIG. 12 shows an illustrative embodiment of an endovascular device according to the described invention. 1310 is an illustration of inflatable balloons attached around the circumference of the endovascular device located proximal to the side-hole (310), (1470).

FIG. 13A and FIG. 13B show an illustrative embodiment of an endovascular device according to the described invention. 1410 is an illustration of a luer lock. 1420 identifies the beginning of the outer catheter. 1430 is a general illustration of grips. 1431 is an illustration of grips on the inner catheter. 1432 is an illustration of grips on the outer catheter. 1440 is an illustration of an outer catheter. 1450 is an illustration of an angled extension protruding from the side-hole (1470) positioned at the base of an aneurysm; length of the angled extension is less than the length of the side-hole (1470). 1460 is an illustration of an inner catheter of the endovascular device. 1480 is an illustration of the distal support segment of the endovascular device.

FIG. 14 shows an illustrative embodiment of an endovascular device (1400) according to the described invention. This embodiment shows no distal support segment. 1310 illustrates an array of inflatable balloons attached around the circumference of the endovascular device.

FIG. 15 shows an illustrative embodiment of a cross-section through the array of five (5) balloons (1310) depicted in side view in FIG. 14 at the proximal end of the endovascular device. This embodiment shows a working lumen (1510) and five (5) fluid channels to the five separate balloons.

### DETAILED DESCRIPTION OF THE INVENTION

### Glossary

The term “ablation” as used herein, refers to a procedure that uses radiofrequency energy (e.g., microwave heat) to destroy a small area of heart tissue that is causing rapid and irregular heartbeats. Destroying this tissue restores the heart's regular rhythm. The procedure is also called radiofrequency ablation.

The terms “acute angle” and “acute angulation” are used interchangeably herein to refer to a sharp, obstructive or abnormal angle or bend (e.g., less than 90 degrees) in an organ, artery, vessel, etc.

### Anatomical Terms:

When referring to animals that typically have one end with a head and mouth, with the opposite end often comprising the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The terms “anomaly”, “variation”, “abnormality” and “aberration” are used interchangeably herein to refer to a deviation from what is standard, normal or expected. For example, “bovine arch variation” is an anatomical deviation from the most common aortic arch branching pattern in humans. By way of additional example, an anomaly can occur in a blood vessel comprising tortuosity.

The term “aneurysm”, as used herein, refers to a localized widening (dilatation) of an artery, a vein, or the heart. At the point of an aneurysm, there is typically a bulge, where the wall of the blood vessel or organ is weakened and may rupture.

Blood flow in most aneurysms is regular and predictable primarily according to the geometric relationship between the aneurysm and its parent artery. As blood flows within the parent artery with an aneurysm, divergence of blood flow, as occurs at the inlet of the aneurysm, leads to dynamic disturbances, producing increased lateral pressure and retrograde vortices that are easily converted to turbulence. Blood flow proceeds from the parent vessel into the aneurysm at the distal or downstream extent of the aneurysm neck (i.e., the transition from the sac to the parent artery), circulates around the periphery along the aneurysm wall from the neck to the top of the fundus (i.e., aneurysm sac) (downstream to upstream), returning in a type of “isotropic shower” along the aneurysm wall toward the neck region, and exits the closest extent of the aneurysm neck into the parent vessel (See, e.g., Strother C. M. Neuroradiology 1994; 36: 530-536; Moulder P. V. Physiology and biomechanics of aneurysms. In: Kerstein M D., Moulder P V, Webb W R, eds. Aneurysms. Baltimore, Md: Williams & Wilkins; 1983:20).

As flow persists, areas of stagnation or vortices develop within a central zone of the aneurysm. These rotating vortices, formed at the entrance to the aneurysm at each systole (i.e., ventricle contraction) and then circulated around the aneurysm, are caused by the slipstreams or regions of recirculating flow rolling upon themselves when they enter the aneurysm at its downstream wall during systole. The stagnant vortex zone occurs in the center and at the fundus or upper portion of the aneurysm and becomes more pronounced in larger aneurysms. It is this stagnant zone that is believed to promote the formation of thrombi or blood clots, particularly in giant aneurysms (See, e.g., Gobin Y. P. et al. Neuroradiology 1994; 36: 530-536; Hademenos G. J. and Massoud T. F. Stroke 1997; 28: 2067-2077).

The term “abdominal aortic aneurysm” or “AAA”, as used herein refers to an aortic diameter at least one and one-half times the normal diameter at the level of the renal arteries, which is approximately 2.0 cm. Generally, a segment of abdominal aorta with a diameter of greater than 3.0 cm is considered an aortic aneurysm. Aortic aneurysms constitute the 14th leading cause of death in the United States. Risk factors associated with AAA include age, sex, ethnicity, smoking, hypertension and atherosclerosis, among others (See, e.g., Aggarwal S. et al. Exp Clin Cardiol. 2011; 16(1): 11-15; Ouriel K. et al. J Vasc Surg. 1992; 15: 12-18; Silverberg E. et al. CA Cancer J Clin. 1990; 40: 9-26).

The term “arteriovenous malformation” (“AVM”), as used herein, refers to a tangle of abnormal and poorly formed blood vessels (e.g., arteries and veins) which have a higher than normal rate of bleeding compared to normal blood vessels.

AVMs are congenital vascular lesions that occur as a result of capillary mal-development between the arterial and venous systems. Approximately 0.14% of the United States population has an intracranial AVM that poses a significant risk and represents a major life threat, particularly to persons under the age of 50 years. The vessels constituting the AVM are weak and enlarged and serve as direct shunts for blood flow between the high-pressure arterial system and the low-pressure venous system, corresponding to a large pressure gradient and small vascular resistance. The abnormal low-resistance, high-flow shunting of blood within the brain AVM without an intervening capillary bed causes the fragile dilated vessels in the nidus (i.e., tangle of blood vessels) to become structurally abnormal and fatigued, to further enlarge, and to rupture (See, e.g., Wilkins R. H. Neurosurgery 1985; 16:421-430; Graves V. B. et al. Invest Radiol. 1990; 25: 952-960; Hademenos G. J. et al., Neurosurgery 1996; 38: 1005-1015).

The abnormal microvessels of an AVM serve as passive conduits for blood flow from the arterial circulation directly to the venous circulation, by-passing their normal physiological function of brain tissue perfusion. The hemodynamic consequences of an AVM occur as a result of two interdependent circulatory mechanisms involved in the shunting of blood between artery and vein (See, e.g., Hademenos G. J. and Massoud T. F. Stroke 1996; 27: 1072-1083).

In the normal cerebral circulation, blood flows under a high cerebrovascular resistance and high cerebral perfusion pressure. However, the presence of a brain AVM in the normal circulation introduces a second abnormal circuit of cerebral blood flow where the blood flow is continuously shunted under a high perfusion pressure through the AVM, possessing a low cerebrovascular resistance and low venous pressure. The clinical consequence of the abnormal shunt is a significant increase in blood returning to the heart (approximately 4 to 5 times the original amount, depending on the diameter and size of the shunt), resulting in a dangerous overload of the heart and cardiac failure. Volumetric blood flow through an AVM ranges from 200 mL/min to 800 mL/min and increases according to nidus size (See, e.g., Yamada S. Neurol Res. 1993; 15: 379-383).

The abnormal shunting of blood flow by brain AVMs rapidly removes or “steals” blood from the normal cerebral circulation and substantially reduces the volume of blood reaching the surrounding normal brain tissue. This phenomenon, known as cerebrovascular steal, depends on the size of the AVM and is the most plausible explanation for the development of progressive neurological deficits. Cerebrovascular steal could translate into additional neurological complications developed as a result of cerebral ischemia or stroke in neuronal territories adjacent to an AVM (See, e.g., Manchola I. F. et al. Neurosurgery 1993; 33: 556-562; Hademenos G. J. and Massoud T. F. Stroke 1997; 28: 2067-2077).

The term “atherectomy”, as used herein, refers to a minimally invasive endovascular surgery technique for removing atherosclerosis from blood vessels within the body by cutting plaque from the walls of a blood vessel.

The term “atherosclerosis” (also known as “hardening of the arteries”), as used herein, refers to a pathological process in which calcified lipid or fatty deposits from flowing blood accumulate along the innermost intimal layer of a vessel wall. Atherosclerotic plaques are found almost exclusively at the outer wall of one or both daughter vessels at major arterial bifurcations, including the carotid. Atherosclerosis and the development of arterial plaques are the products of a host of independent biochemical processes including the oxidation of low-density lipoproteins, formation of fatty streaks, and the proliferation of smooth muscle cells. As the plaques form, the walls become thick, fibrotic, and calcified. As a result, the segment narrows, reducing the flow of blood to the tissues supplied by the artery (See, e.g., Hademenos G. J. and Massoud T. F. Stroke 1997; 28: 2067-2077; Hademenos G. J. Am Scientist 1997; 85: 226-235; Woolf N., Davies M. J. Sci Am Science & Medicine 1994; 1: 38-47).

Atherosclerotic deposits promote the development of blood clots or the process of thrombosis, due in part, to flow obstruction and to high shear stresses exerted on the vessel wall by the blood. High wall shear stress mechanically damages the inner wall of the artery, initiating a lesion. Low wall shear stress encourages the deposition of particles on the artery wall, promoting the accumulation of plaque. Turbulence has also been implicated in atherosclerotic disease because it can increase the kinetic energy deposited in the vessel walls and because it can lead to areas of stasis, or stagnant blood flow, that promote clotting. The presence of atherosclerotic lesions introduces an irregular vessel surface, resulting in turbulent blood flow, thus causing the dislodgment of plaques of varying size into the bloodstream. Subsequently, the dislodged plaque lodges into a vessel of smaller size, preventing further passage of blood flow (See, e.g., Hademenos G. J. and Massoud T. F. Stroke 1997; 28: 2067-2077).

The term “atresia”, as used herein, refers to the absence or abnormal narrowing of an opening or passage in the body. For example, aortic atresia refers to a rare congenital anomaly in which the aortic orifice is absent or closed.

The term “atrial fibrillation”, as used herein, refers to an irregular and often fast heart rate which may cause symptoms such as heart palpitations, fatigue, and shortness of breath. Atrial fibrillation weakens the cardiac wall and introduces abnormalities in the physiological function of the heartbeat, which ultimately result in reduced systemic pressure, conditions of ischemia and stroke.

The term “brachiocephalic trunk”, also known as “innominate artery”, as used herein, refers to a major vessel that supplies the head, neck and right arm. It is the first of three main branches of the aortic arch, which originates from the upward convexity. After arising in the midline, it courses upwards to the right, crossing the trachea, and bifurcates posteriorly to the right sternoclavicular joint into the right subclavian and right common carotid arteries. It typically measures 4-5 cm in length with a diameter of approximately 12 mm.

The term “brain aneurysm”, as used herein, refers to a cerebrovascular disease that manifests as a pouching or ballooning of the vessel wall (i.e., vascular dilation). The vascular dilatation develops at a diseased site along the arterial wall into a distended sac of stressed and thinned arterial tissue. The fully developed cerebral aneurysm typically ranges in size from a few millimeters to 15 mm but can attain sizes greater than 2.5 cm. If left untreated, the aneurysm may continue to expand until it ruptures, causing hemorrhage, severe neurological complications and deficits, and possibly death (Hademenos G. J. and Massoud T. F. Stroke 1997; 28: 2067-2077; Hademenos G. J. Phys Today 1995; 48: 24-30).

The two main treatment options for a patient suffering from a brain aneurysm are (i) surgical clipping; and (ii) endovascular coiling. Surgical clipping is an intracranial procedure in which a small metallic clip is placed along the neck of the aneurysm. The clip prevents blood from entering into the aneurysm sac so that it no longer poses a risk for bleeding. The clip remains in place, causing the aneurysm to shrink and permanently scar. Endovascular coiling is a minimally invasive technique in which a catheter is inserted into the femoral artery and navigated through the blood vessels to the vessels of the brain and into the aneurysm. Coils are then packed into the aneurysm to the point where it arises from the blood vessel, thus preventing blood flow from entering the aneurysm. Additional devices, such as a stent or balloon, for example, can be needed to keep the coils in place.

The term “branch”, as used herein, refers to something that extends from or enters into a main body or source; a division or offshoot from a main stem (e.g., blood vessels); one of the primary divisions of a blood vessel.

The term “coarctation” or “coarctation of the aorta”, as used herein, refers to a congenital narrowing of a short section of the aorta.

The terms “compound curves” and “multi-curves” are used interchangeably herein to refer to multiple deflection points along the length of a catheter. By way of example, two deflection points allow a catheter to be deflected into an “S” shape or the shape of a shepherd's hook.

The term “conduit”, as used herein, refers to a tube through which something passes.

The term “curve diameter”, as used herein, refers to the furthest distance a catheter moves from its straight axis as it is being deflected. The curve diameter does not always remain constant during deflection and does not necessarily indicate the location of the catheter tip.

The term “dilator”, as used herein, refers to a long, tapered device adapted to stretch an opening in skin and/or to stretch a blood vessel to allow for insertion of a larger device, e.g., a sheath, catheter, etc.

The term “distal”, as used herein, refers to the state of being situated away from the point of attachment or origin.

The term “deflection”, as used herein, refers to movement of a catheter tip independent of the rest of the catheter.

The term “dyscrasia”, as used herein, refers to an abnormal or disordered state of the body or a bodily part. The term “blood dyscrasia”, as used herein, refers to an abnormally of blood cells or of clotting elements.

The term “embolus” (plural “emboli”), as used herein, refers to a gaseous or particulate matter that acts as a traveling “clot”. A common example of an embolus is a platelet aggregate dislodged from an atherosclerotic lesion. The dislodged platelet aggregate is transported by the bloodstream through the cerebrovasculature until it reaches a vessel too small for further propagation. The clot remains there, clogging the vessel and preventing blood flow from entering the distal vasculature. Emboli can originate from distant sources such as the heart, lungs, and peripheral circulation, which could eventually travel within the cerebral blood vessels, obstructing flow and causing stroke. Other sources of emboli include atrial fibrillation and valvular disease. The severity of stroke depends on the size of the embolus and the location of the obstruction. The bigger the embolus and the larger the vessel obstruction, the larger the territory of brain at risk (Hademenos G. J. and Massoud T. F. Stroke 1997; 28: 2067-2077).

The term “endoluminal”, as used herein, refers to the state of being within a tubular organ or structure (e.g., blood vessel, duct, gastrointestinal tract, etc.) or within a segment. The term “segment”, as used herein, refers to the inner open space or cavity of a tubular structure.

The term “French” (abbreviated “Fr” or “F” or “Fg” or “Ga” or “CH” or “Ch”), as used herein, is a system used to measure the diameter of a catheter. The French unit of measure is equivalent to three times the diameter in millimeters (mm). For example, 9 Fr is equivalent to a diameter of 3 mm.

The term “hemorrhage”, as used herein, refers to the escape of blood from a ruptured blood vessel.

Blood vessels are typically structurally adept to withstand the dynamic quantities required to maintain circulatory function. For reasons that are not entirely understood, the vessel wall can become fatigued and abnormally weak and possibly rupture. With vessel rupture, hemorrhage occurs with blood seeping into the surrounding brain tissue. As the blood accumulates within the brain, the displaced volume causes the blood, now thrombosed, to ultimately compress the surrounding vessels. The compression of vessels translates into a reduced vessel diameter and a corresponding reduction in flow to surrounding tissue, thereby enlarging the insult (See, e.g., Hademenos G. J. and Massoud T. F. Stroke 1997; 28: 2067-2077).

In the brain, hemorrhage may occur at the brain surface (extraparenchymal), for example, from the rupture of congenital aneurysms at the circle of Willis, causing subarachnoid hemorrhage (SAH). Hemorrhage also can be intraparenchymal, for example, from rupture of vessels damaged by long-standing hypertension, and may cause a blood clot (intracerebral hematoma) within the cerebral hemispheres, in the brain stem, or in the cerebellum. Hemorrhage can be accompanied by ischemia or infarction. The mass effect of an intracerebral hematoma may compromise the blood supply of adjacent brain tissue; or SAH may cause reactive vasospasm of cerebral surface vessels, leading to further ischemic brain damage. Infarcted tissue may also become secondarily hemorrhagic. Among the vascular lesions that can lead to hemorrhagic strokes are aneurysms and arteriovenous malformations (AVMs) (See, e.g., Hademenos G. J. and Massoud T. F. Stroke 1997; 28: 2067-2077).

The term “hypoplasia”, as used herein, refers to a condition of arrested development in which an organ or other part of the body remains below the normal size or in an immature state, usually due to a deficiency in the number of cells; atrophy due to destruction of some of the elements and not merely to their general reduction in size.

The term “introducer”, as used herein, refers to an instrument such as a tube or a sheath that is placed within a vein or artery for introduction of a flexible device, for example, a catheter, needle, wire, etc.

The terms “ischemic” and “ischemia”, as used herein, refer to deficient supply of blood to a body part generally due to obstruction of the inflow of arterial blood (e.g., by the narrowing of arteries, spasm or disease).

The term “kickback”, as used herein refers to the phenomenon of catheter coil prolapse (slipping forward or down) due to a counterforce against the catheter by the prolapsed coil tail. The counterforce can be due to a lack of available space to insert the last coil. This lack of space can be the result of, for example, a blood vessel variation such as a bovine arch variation, a vertebral artery variation, a thrombus, an embolus, or an arteriovenous malformation.

The term “myocardial infarction”, as used herein, refers to death of cells of an area of heart muscle as a result of oxygen deprivation, which in turn is caused by obstruction of the blood supply; commonly referred to as a “heart attack”. The most common cause is thrombosis of an atherosclerotic coronary artery or a spasm. Less common causes included coronary artery abnormalities and vasculitis (inflammation of blood vessels).

The term “proximal”, as used herein, refers to the state of being situated next to or nearest the point of attachment or origin.

The term “recanalization”, as used herein, refers to the process of restoring flow to or reuniting an interrupted channel of a bodily tube (e.g., a blood vessel).

The term “reperfusion”, as used herein, refers to restoration of the flow of blood to a previously ischemic organ or tissue (e.g., heart or brain).

The term “restenosis”, as used herein, refers to the recurrence of abnormal narrowing of a blood vessel (e.g., artery or vein) or valve.

The term “steerability”, as used herein, refers to an ability to turn or rotate the distal end of a catheter with like-for-like movement of the proximal section or the catheter handle.

The term “stroke” or “cerebrovascular accident”, as used herein, refers to neurological signs and symptoms, usually focal and acute, which result from diseases involving blood vessels. Strokes are either occlusive (due to closure of a blood vessel) or hemorrhagic (due to bleeding from a vessel). Although most occlusive strokes are due to atherosclerosis and thrombosis, and most hemorrhagic strokes are associated with hypertension or aneurysms, strokes of either type may occur at any age from many causes, including cardiac disease, trauma, infection, neoplasm, blood dyscrasia, vascular malformation, immunological disorder, and exogenous toxins. An ischemia stroke results from a lack of blood supply and oxygen to the brain that occurs when reduced perfusion pressure distal to an abnormal narrowing (stenosis) of a blood vessel is not compensated by autoregulatory dilation of the resistance vessels. When ischemia is sufficiently severe and prolonged, neurons and other cellular elements die. This condition is referred to as “infarction” (See, e.g., Hart R. G. et al., Stroke 1990; 21:1111-1121). Although the consequences of both ischemic and hemorrhagic stroke are similar (i.e., vessel obstruction, resultant reduced blood flow to the brain, neurological deficits and possibly death), the biophysical and hemodynamic mechanisms behind the obstruction of blood flow are different. Biophysical mechanisms for the development of obstructions that ultimately lead to stroke can arise by six distinct processes: atherosclerosis, embolus, thrombus, reduced systemic pressure, hemorrhage, and vasospasm (See, e.g., Hademenos G. J. and Massoud T. F., Stroke 1997; 28: 2067-2077).

The term “taper”, as used herein, refers to a reduction of thickness and/or diameter toward one end, or the gradual diminution of width or thickness in an elongated object; i.e., to become more slender toward one end.

The term “thrombectomy”, as used herein, refers to the surgical excision of a thrombus.

The term “thrombus”, as used herein, refers to an internal physiological mechanism responsible for the clotting of blood. A thrombus is a blood clot, an aggregation of platelets and fibrin formed in response either to an atherosclerotic lesion or to vessel injury. In response to vessel or tissue injury, the blood coagulation system is activated, which initiates a cascade of processes, transforming prothrombin, ultimately resulting in a fibrin clot:

(Prothrombin Thrombins→Fibrinogen→Fibrin→Fibrin Clot)→(See, e.g., Hademenos G. J. and Massoud T. F. Stroke 1997; 28: 2067-2077).

Although a host of mechanisms and causes are responsible for vessel injury, vessel injury can occur as a result of forces (e.g., shear stresses) coupled with excess energy created by the turbulent flow exerted against the inner (intimal) lining of the vessel wall, particularly an atherosclerotic vessel wall (See, e.g., Fry D. L. Circ Res. 1968; 22: 165-197; Stein P. D. and Sabbah H. N. Circ Res. 1974; 35: 608-614; Mustard J. F. et al. Am J Med. 1962; 33: 621-647; Goldsmith H. L. et al. Thromb Haemost 1986; 55: 415-435).

The term “tortuosity” and other grammatical forms of the term “tortuous” is used herein to refer to a property of a tube, passage or blood vessel (e.g., an artery or a vein) being twisted, crooked or comprising many turns.

The term “vasospasm”, as used herein, refers to the sudden constriction of a blood vessel, reducing its diameter and flow rate. When bleeding occurs in the subarachnoid space, the arteries in the subarachnoid space can become spastic with a muscular contraction, known as cerebral vasospasm. The contraction from vasospasm can produce a focal constriction of sufficient severity to cause total occlusion. The length of time that the vessel is contracted during vasospasm varies from hours to days. However, regardless of the duration of vessel constriction, reduction of blood flow induces cerebral ischemia, thought to be reversible within the first 6 hours and irreversible thereafter. It has been shown that vasospasm is maximal between 5 and 10 days after subarachnoid hemorrhage and can occur up to 2 weeks after subarachnoid hemorrhage (See, e.g., Wilkins R. H. Contemp Neurosurg. 1988; 10:1-66; Hademenos G. J. and Massoud T. F. Stroke 1997; 28: 2067-2077).

In the various views of the drawings, like reference characters designate like or similar parts.

While size can vary, each of the described endovascular devices herein described share the following general features: a working segment, a support segment, a working lumen, a support lumen, a side hole, an angled extension.

According to some embodiments, the distal segment beyond the side hole (the support segment) for each of the described devices is effective to provide stability to each endovascular device, to provide strength to the endovascular device, to provide support for the endovascular device, to facilitate placement of the endovascular device, to anchor the endovascular device within a blood vessel, to reduce kickback of the endovascular device, or a combination thereof.

According to some embodiments, the endovascular devices described comprise additional support elements, for example, a balloon, a stent, a wire, or a combination thereof. According to some embodiments, the additional support element(s) is/are effective: to provide stability to the endovascular device, strength to the endovascular device, to provide support for the endovascular device, to facilitate placement of endovascular device; to anchor the endovascular device within a blood vessel; to provide stability to the working segment; to provide strength to the working segment, to provide support for the working segment; to facilitate placement of the working segment in a blood vessel; to anchor the working segment within a blood vessel or a combination thereof.

According to some embodiments, the endovascular device comprises inflatable balloons with two dimensions, a diameter and a length, sized to fit within the diameter of a vessel.

According to some embodiments,an inflatable balloon can be comprised of various shapes including, but not limited to, a cylindrical shape, a spherical shape, an oval shape, a conical shape, a stepped shape, a tapered shape and a dog bone shape.

According to some embodiments, the inflatable balloon is comprised of a material such as, for example, a polyamide, polyethylene terephthalate (PET), polyurethane, composites, engineered nylons and equivalent materials. Engineered nylons include, but are not limited to, Pebax®, Grilamid®, and Vestamid®.

According to some embodiments, the inflatable balloon ends can be comprised of various shapes including, but not limited to, a conical sharp corner end, a conical radius corner end, an offset neck end, a spherical end and a square end.

According to some embodiments, the inflatable balloon is filled with a fluid. Non-limiting examples of the fluid include sterile water and saline.

According to some embodiments, the stent is a retrievable stent. According to some embodiments, the stent is capable of being advanced into a blood vessel through the lumen defined in the support segment. According to some embodiments, the retrievable stent is attached to the distal portion of the support segment of the endovascular device. According to some embodiments, the retrievable stent is attached to a second device separate from the endovascular device. According to some embodiments, the second separate device separate is capable of being advanced into a blood vessel through the lumen defined in the support segment of the endovascular device.

According to some embodiments, the second separate device is a diagnostic catheter, a therapeutic catheter or other therapeutic device.

Diagnostic catheters include, but are not limited to, angiography catheters, electrophysiology catheters, and intravenous ultrasound catheters.

Catheter angiography can be performed using such techniques as, for example, X-rays, computed tomography (CT) and magnetic resonance imaging (MRI). In catheter angiography, a catheter is inserted into a blood vessel (e.g., an artery) through a small incision in the skin. The catheter is guided to the area being examined, a contrast material is injected through the catheter and images are acquired using a small dose of ionizing radiation (e.g., X-rays). Contrast agents include, but are not limited to, iodinated low-osmolar contrast media (LOCM) and high-osmolar contrast media (HOCM). Low-osmolar contrast media include, but are not limited to, ioxaglate, iopamidol, iohexol, ioidixanol, iotrolan, ioxaglate, ioxilan, iopromide, ioversol and iomeprol. Non-limiting examples of high-osmolar contrast media include diatrizoate, metrizoate and iothalamate.

Catheter electrophysiology is an invasive heart catheterization that is designed to evaluate the electrical system of the heart. This test evaluates if there is a need for implantation of a pacemaker or defibrillator or to perform a catheter ablation which is a procedure that uses radiofrequency energy (similar to microwave heat) to destroy small areas of heart tissue that cause rapid or irregular heartbeats. In this procedure, a catheter is introduced into a blood vessel and placed under X-ray guidance into the heart. For example, catheter electrophysiology is used for evaluating patients who have concerning symptoms such as fainting, episodes of almost fainting, sensations of rapid heartbeats, or excessively slow heartbeats.

Ultrasound catheterization, or intravascular ultrasound (IVUS) is an imaging procedure using a catheter with a miniaturized ultrasound probe attached to the distal end. The proximal end of the catheter is attached to computerized ultrasound equipment which measure how sound waves reflect off blood vessels and converts these measurements into images. IVUS is used to determine, among others, the accumulation of plaque in an artery and the correct placement of a stent.

The term “therapeutic catheter” includes, but is not limited to, a proximal endovascular thrombectomy catheter, a distal endovascular thrombectomy catheter, a self-expanding stent catheter, a retrievable thrombectomy stent catheter, an ablation catheter, a percutaneous transluminal angioplasty (PTCA) catheter, an embolization catheter, a coil, a liquid embolic, a stent, a flow-diverting stent, and an intravascular web device.

PTCA is a minimally invasive procedure to open blocked coronary arteries, allowing blood to circulate unobstructed to the heart muscle. The procedure begins with the injection of local anesthesia into the groin area and putting a needle into the femoral artery. A guide wire is placed through the needle and the needle is removed. An introducer is then placed over the guide wire, after which the wire is removed. A different sized guide wire is then put in its place. Next, a long narrow tube called a diagnostic catheter is advanced through the introducer over the guide wire, into the blood vessel. This catheter is then guided to the aorta and the guide wire is removed. Once the catheter is placed in the opening (or ostium) of one the coronary arteries, a contrast dye is injected and an x-ray is taken. If a treatable blockage is noted, the first catheter is exchanged for a guiding catheter. Once the guiding catheter is in place, a guide wire is advanced across the blockage, then a balloon catheter is advanced to the blockage site. The balloon is inflated for a few seconds to compress the blockage against the artery wall. Then the balloon is deflated.

Catheter embolization is a minimally invasive treatment that occludes or blocks one or more blood vessels or vascular channels of malformations (abnormalities). In a catheter embolization procedure, medications or synthetic materials (embolic agents) are placed through a catheter into a blood vessel to prevent blood flow to the area. Using image-guidance, a catheter is inserted through the skin to the treatment site. A contrast material is then injected through the catheter and a series of x-rays are taken to locate the exact site of bleeding or abnormality. Next, a medication or an embolic agent is injected through the catheter. Additional x-rays are taken to ensure the loss of blood flow in the target vessel or malformation. Uses of catheter embolization include, but are not limited, control or prevention of abnormal bleeding, including bleeding that results from an injury, tumor or gastrointestinal tract lesions such as an ulcer or diverticular disease; controlling bleeding into the abdomen or pelvis caused by traumatic injuries; treatment of long menstrual periods or heavy menstrual bleeding that results from fibroid tumors of the uterus; to occlude or close off vessels that are supplying blood to a tumor; to eliminate an arteriovenous malformation (AVM) or arteriovenous fistula (AVF) (abnormal connection or connections between arteries and veins); and to treat aneurysms (a bulge or sac formed in a weak artery wall) by either blocking an artery supplying the aneurysm or closing the aneurysmal sac itself.

The various components of the described invention can be comprised of one or more materials. For example, according to some embodiments, the components can comprise one or more of a thermoplastic, a thermoset, a composite or a radiopaque filler.

Thermoplastics include, but are not limited to, nylon, polyethylene terephthalate (PET), urethane, polyethylene, polyvinyl chloride (PVC) and polyether ether ketone (PEEK).

Thermosets include, but are not limited to, silicone, polytetrafluoroethylene (PTFE) and polyimide.

Composites include, but are not limited to, liquid crystal polymers (LCP). LCPs are partially crystalline aromatic polyesters based on p-hydroxybenzoic acid and related monomers. LCPs are highly ordered structures when in the liquid phase, but the degree of order is less than that of a regular solid crystal. LCPs can be substituted for such materials as ceramics, metals, composites and other plastics due to their strength at extreme temperatures and resistance to chemicals, weathering, radiation and heat. Non-limiting examples of LCPs include wholly or partially aromatic polyesters or copolyesters such as XYDAR® (Amoco) or VECTRA® (Hoechst Celanese). Other commercial liquid crystal polymers include SUMIKOSUPER™ and EKONOL™ (Sumitomo Chemical), DuPont HX™ and DuPont ZENITE™ (E.I. DuPont de Nemours), RODRUN™ (Unitika) and GRANLAR™ (Grandmont).

Non-limiting examples of radiopaque fillers include barium sulfate, bismuth oxychloride, and tantalum.

According to some embodiments, the wire is capable of being advanced into a blood vessel through the lumen defined in the support segment. According to some embodiments, the wire is comprised of a core material which includes, but is not limited to, stainless steel, nitinol or a combination thereof. In general, stainless steel is easier to torque and is more rigid, providing better columnar support. Nitinol is more flexible and kink resistant. Developments such as high-tensile-strength stainless steel and combinations of stainless steel with nitinol have been utilized. High-tensile-strength stainless steel provides more column strength and torquability than original stainless steel. The use of hybrid wires incorporates high-tensile stainless steel shafts with nitinol tips to impart high torquability and columnar shaft strength with kink-resistance tips.

According to some embodiments, the wire comprises a core taper. The core tapers are areas where the core of the wire changes over a set distance. There may becan be several tapers in a wire. Long, gradual tapers track well around bends, but do not provide as much support in short distances. Broad, gradual, or long tapers offer acute vessel access and improved tracking. Devices with abrupt or short tapers create support in shorter distances and have a greater tendency to prolapse.

According to some embodiments, the wire comprises a core grind (i.e., constant diameter).

According to some embodiments, the wire comprises a core that extends to the tip. A core that extends to the tip of the wire increases the transmission of force, is more durable and steerable, improves tactile feedback, and is used in peripheral vessels. According to some embodiments, the wire comprises a core that does not extend to the tip. A core that does not extend to the tip (i.e., shaping ribbon design) is delicate, flexible, and soft. This kind of tip is also easier to shape, can be easily prolapsed, and is less likely to inadvertently injure distal vessels.

According to some embodiments, the wire comprises a cover. Covers included, but are not limited to, a polymer or a plastic. A sleeve of polymer or plastic placed over the wire core enhances lubricity which results in less drag, enhanced lesion crossing, and smooth tracking in tortuous vessels. According to some embodiments, the wire comprises a coating. Non-limiting examples of a coating include a hydrophobic coating and a hydrophilic coating. Hydrophobic coatings reduce friction and improve device trackability by repelling water to create a smooth, “wax-like” surface, with no water actuation required. Hydrophilic coatings attract water to create a slippery, “gel-like” surface.

According to some embodiments, the wire is a guide wire.

According to some embodiments, the side-hole of the endovascular device comprises an angled extension extending from the side-hole defining a lumen, with the angled extension at least partially disposed within the lumen of the working segment. According to some embodiments, the angled extension the angled extension facilitates the direction in which the catheter is going to go, and therefore helps the second catheter or device make the turn into the desired vessel to be accessed. According to some embodiments, the length of the angled extension is no longer than the length of the side hole. It is understood that a catheter with an angled extension cannot be introduced into the body with the angled extension in an extended state. Instead, a dilator or introducer must extend through the side hole. It follows that the angled extension cannot be longer than the side hole or it would not be able to be extended from the side hold once the catheter is deployed.

It should be understood that any catheter with a side hole, including, without limitation, each of catheters 100, 200, 600 and 1400, can have an angled extension. When an angled extension is incorporated, the length of the side-hole is equal to or larger than the length of the soft angled extension of the catheter.

According to some embodiments, the angle of the angled extension is fixed. According to some embodiments, the angle of the angled extension ranges from about 0 degrees to about 359 degrees. According to some embodiments, the angle of the angled extension ranges from about 0 degrees to at least 359 degrees. According to some embodiments, the angle of the angled extension ranges from at least 0 degrees to at least 359 degrees. According to some embodiments, the angle of the angled extension is at least 0 degrees. According to some embodiments, the angle of the angled extension is at least 10 degrees. According to some embodiments, the angle of the angled extension is at least 20 degrees. According to some embodiments, the angle of the angled extension is at least 30 degrees. According to some embodiments, the angle of the angled extension is at least 40 degrees. According to some embodiments, the angle of the angled extension is at least 50 degrees. According to some embodiments, the angle of the angled extension is at least 60 degrees. According to some embodiments, the angle of the angled extension is at least 70 degrees. According to some embodiments, the angle of the angled extension is at least 80 degrees. According to some embodiments, the angle of the angled extension is at least 90 degrees. According to some embodiments, the angle of the angled extension is at least 100 degrees. According to some embodiments, the angle of the angled extension is at least 110 degrees. According to some embodiments, the angle of the angled extension is at least 120 degrees. According to some embodiments, the angle of the angled extension is at least 130 degrees. According to some embodiments, the angle of the angled extension is at least 140 degrees. According to some embodiments, the angle of the angled extension is at least 150 degrees. According to some embodiments, the angle of the angled extension is at least 160 degrees. According to some embodiments, the angle of the angled extension is at least 170 degrees. According to some embodiments, the angle of the angled extension is at least 180 degrees. According to some embodiments, the angle of the angled extension is at least 190 degrees. According to some embodiments, the angle of the angled extension is at least 200 degrees. According to some embodiments, the angle of the angled extension is at least 210 degrees. According to some embodiments, the angle of the angled extension is at least 220 degrees. According to some embodiments, the angle of the angled extension is at least 230 degrees. According to some embodiments, the angle of the angled extension is at least 240 degrees. According to some embodiments, the angle of the angled extension is at least 250 degrees. According to some embodiments, the angle of the angled extension is at least 260 degrees. According to some embodiments, the angle of the angled extension is at least 270 degrees. According to some embodiments, the angle of the angled extension is at least 280 degrees. According to some embodiments, the angle of the angled extension is at least 290 degrees. According to some embodiments, the angle of the angled extension is at least 300 degrees. According to some embodiments, the angle of the angled extension is at least 310 degrees. According to some embodiments, the angle of the angled extension is at least 320 degrees. According to some embodiments, the angle of the angled extension is at least 330 degrees. According to some embodiments, the angle of the angled extension is at least 340 degrees. According to some embodiments, the angle of the angled extension is at least 350 degrees. According to some embodiments, the angle of the angled extension is at least 359 degrees.

According to some embodiments, the angle (relative to the length of the tube 360) of the angled extension is adjustable. According to some embodiments, the angle of the angled extension is adjustable from about 0 degrees to about 359 degrees. According to some embodiments, the angle of the angled extension is adjustable from at least 0 degrees to at least 359 degrees. According to some embodiments, the angle of the angled extension is adjustable to at least 0 degrees. According to some embodiments, the angle of the angled extension is adjustable to at least 10 degrees. According to some embodiments, the angle of the angled extension is adjustable to at least 20 degrees. According to some embodiments, the angle of the angled extension is adjustable to at least 30 degrees. According to some embodiments, the angle of the angled extension is adjustable to at least 40 degrees. According to some embodiments, the angle of the angled extension is adjustable to at least 50 degrees. According to some embodiments, the angle of the angled extension is adjustable to at least 60 degrees. According to some embodiments, the angle of the angled extension is adjustable to at least 70 degrees. According to some embodiments, the angle of the angled extension is adjustable to at least 80 degrees. According to some embodiments, the angle of the angled extension is adjustable to at least 90 degrees. According to some embodiments, the angle of the angled extension is adjustable to at least 100 degrees. According to some embodiments, the angle of the angled extension is adjustable to at least 110 degrees. According to some embodiments, the angle of the angled extension is adjustable to at least 120 degrees. According to some embodiments, the angle of the angled extension is adjustable to at least 130 degrees. According to some embodiments, the angle of the angled extension is adjustable to at least 140 degrees. According to some embodiments, the angle of the angled extension is adjustable to at least 150 degrees. According to some embodiments, the angle of the angled extension is adjustable to at least 160 degrees. According to some embodiments, the angle of the angled extension is adjustable to at least 170 degrees. According to some embodiments, the angle of the angled extension is adjustable to at least 180 degrees. According to some embodiments, the angle of the angled extension is adjustable to at least 190 degrees. According to some embodiments, the angle of the angled extension is adjustable to at least 200 degrees. According to some embodiments, the angle of the angled extension is adjustable to at least 210 degrees. According to some embodiments, the angle of the angled extension is adjustable to at least 220 degrees. According to some embodiments, the angle of the angled extension is adjustable to at least 230 degrees. According to some embodiments, the angle of the angled extension is adjustable to at least 240 degrees. According to some embodiments, the angle of the angled extension is adjustable to at least 250 degrees. According to some embodiments, the angle of the angled extension is adjustable to at least 260 degrees. According to some embodiments, the angle of the angled extension is adjustable to at least 270 degrees. According to some embodiments, the angle of the angled extension is adjustable to at least 280 degrees. According to some embodiments, the angle of the angled extension is adjustable to at least 290 degrees. According to some embodiments, the angle of the angled extension is adjustable to at least 300 degrees. According to some embodiments, the angle of the angled extension is adjustable to at least 310 degrees. According to some embodiments, the angle of the angled extension is adjustable to at least 320 degrees. According to some embodiments, the angle of the angled extension is adjustable to at least 330 degrees. According to some embodiments, the angle of the angled extension is adjustable to at least 340 degrees. According to some embodiments, the angle of the angled extension is adjustable to at least 350 degrees. According to some embodiments, the angle of the angled extension is adjustable to at least 359 degrees. According to some embodiments, the angled extension is adjusted after the endovascular device 300 is inserted into a blood vessel.

According to some embodiments, the angled extension comprises a shape memory polymer (SMP). Shape memory polymers include, but are not limited to methacrylates, polyurethanes, blends of polystyrene and polyurethane, and polyvinylchloride.

According to some embodiments, the angled extension of the catheter comprises a shape memory alloy (SMA). Non-limiting examples of shape memory alloys include nickel-titanium (i.e., nitinol).

According to some embodiments, an introducer is effective to straighten the angled extension of the endovascular device upon entry into a blood vessel. According to some embodiments, the introducer is sufficiently rigid to straighten the angled extension, but flexible enough to navigate upper cervical and intracranial blood vessels. According to some embodiments, the introducer comprises a dilator. According to some embodiments, the dilator is effective to straighten the angled extension of the endovascular device upon entry into a blood vessel.

According to some embodiments, the angled extension of the endovascular device protrudes from the side-hole when the endovascular device is in its intended position within the blood vessel, when the dilator is removed from the blood vessel, or both. According to some embodiments, the dilator is an inner dilator.

According to some embodiments, the diameter of the side-hole of the endovascular device ranges from about 0.1 French (Fr) (0.001 inches) to about 30 French (Fr) (0.394 inches). According to some embodiments, the diameter of the side-hole of the endovascular device ranges from about 0.1 French (Fr) (0.001 inches) to at least 30 French (Fr) (0.394 inches). According to some embodiments, the diameter of the side-hole of the endovascular device is at least 0.1 French (Fr) (0.001 inches). According to some embodiments, the diameter of the side-hole of the endovascular device is at least 0.2 French (Fr) (0.002 inches). According to some embodiments, the diameter of the side-hole of the endovascular device is at least 0.3 French (Fr) (0.003 inches). According to some embodiments, the diameter of the side-hole of the endovascular device is at least 0.4 French (Fr) (0.005 inches). According to some embodiments, the diameter of the side-hole of the endovascular device is at least 0.5 French (Fr) (0.007 inches). According to some embodiments, the diameter of the side-hole of the endovascular device is at least 0.6 French (Fr) (0.008 inches). According to some embodiments, the diameter of the side-hole of the endovascular device is at least 0.7 French (Fr) (0.009 inches). According to some embodiments, the diameter of the side-hole of the endovascular device is at least 0.8 French (Fr) (0.01 inches). According to some embodiments, the diameter of the side-hole of the endovascular device is at least 0.9 French (Fr) (0.012 inches). According to some embodiments, the diameter of the side-hole of the endovascular device is at least 1 French (Fr) (0.013 inches). According to some embodiments, the diameter of the side-hole of the endovascular device is at least 1.1 French (Fr) (0.014 inches). According to some embodiments, the diameter of the side-hole of the endovascular device is at least 1.2 French (Fr) (0.016 inches). According to some embodiments, the diameter of the side-hole of the endovascular device is at least 1.3 French (Fr) (0.017 inches). According to some embodiments, the diameter of the side-hole of the endovascular device is at least 1.4 French (Fr) (0.018 inches). According to some embodiments, the diameter of the side-hole of the endovascular device is at least 1.5 French (Fr) (0.02 inches). According to some embodiments, the diameter of the side-hole of the endovascular device is at least 1.6 French (Fr) (0.021 inches). According to some embodiments, the diameter of the side-hole of the endovascular device is at least 1.7 French (Fr) (0.022 inches). According to some embodiments, the diameter of the side-hole of the endovascular device is at least 1.8 French (Fr) (0.024 inches). According to some embodiments, the diameter of the side-hole of the endovascular device is at least 1.9 French (Fr) (0.025 inches). According to some embodiments, the diameter of the side-hole of the endovascular device is at least 2 French (Fr) (0.026 inches). According to some embodiments, the diameter of the side-hole of the endovascular device is at least 2.1 French (Fr) (0.028 inches). According to some embodiments, the diameter of the side-hole of the endovascular device is at least 2.2 French (Fr) (0.029 inches). According to some embodiments, the diameter of the side-hole of the endovascular device is at least 2.3 French (Fr) (0.03 inches). According to some embodiments, the diameter of the side-hole of the endovascular device is at least 2.4 French (Fr) (0.031 inches). According to some embodiments, the diameter of the side-hole of the endovascular device is at least 2.5 French (Fr) (0.033 inches). According to some embodiments, the diameter of the side-hole of the endovascular device is at least 2.6 French (Fr) (0.034 inches). According to some embodiments, the diameter of the side-hole of the endovascular device is at least 2.7 French (Fr) (0.035 inches). According to some embodiments, the diameter of the side-hole of the endovascular device is at least 2.8 French (Fr) (0.037 inches). According to some embodiments, the diameter of the side-hole of the endovascular device is at least 2.9 French (Fr) (0.038 inches). According to some embodiments, the diameter of the side-hole of the endovascular device is at least 3 French (Fr) (0.039 inches). According to some embodiments, the diameter of the side-hole of the endovascular device is at least 3.1 French (Fr) (0.041 inches). According to some embodiments, the diameter of the side-hole of the endovascular device is at least 3.2 French (Fr) (0.042 inches). According to some embodiments, the diameter of the side-hole of the endovascular device is at least 3.3 French (Fr) (0.043 inches). According to some embodiments, the diameter of the side-hole of the endovascular device is at least 3.4 French (Fr) (0.045 inches). According to some embodiments, the diameter of the side-hole of the endovascular device is at least 3.5 French (Fr) (0.046 inches). According to some embodiments, the diameter of the side-hole of the endovascular device is at least 3.6 French (Fr) (0.047 inches). According to some embodiments, the diameter of the side-hole of the endovascular device is at least 3.7 French (Fr) (0.049 inches). According to some embodiments, the diameter of the side-hole of the endovascular device is at least 3.8 French (Fr) (0.05 inches). According to some embodiments, the diameter of the side-hole of the endovascular device is at least 3.9 French (Fr) (0.051 inches).

According to some embodiments, the diameter of the side-hole of the endovascular device is at least 4 French (Fr) (0.052 inches). According to some embodiments, the diameter of the side-hole of the endovascular device is at least 4.1 French (Fr) (0.054 inches). According to some embodiments, the diameter of the side-hole of the endovascular device is at least 4.2 French (Fr) (0.055 inches). According to some embodiments, the diameter of the side-hole of the endovascular device is at least 4.3 French (Fr) (0.056 inches). According to some embodiments, the diameter of the side-hole of the endovascular device is at least 4.4 French (Fr) (0.058 inches). According to some embodiments, the diameter of the side-hole of the endovascular device is at least 4.5 French (Fr) (0.059 inches). According to some embodiments, the diameter of the side-hole of the endovascular device is at least 4.6 French (Fr) (0.06 inches). According to some embodiments, the diameter of the side-hole of the endovascular device is at least 4.7 French (Fr) (0.062 inches). According to some embodiments, the diameter of the side-hole of the endovascular device is at least 4.8 French (Fr) (0.063 inches). According to some embodiments, the diameter of the side-hole of the endovascular device is at least 4.9 French (Fr) (0.064 inches). According to some embodiments, the diameter of the side-hole of the endovascular device is at least 5 French (Fr) (0.066 inches). According to some embodiments, the diameter of the side-hole of the endovascular device is at least 5.1 French (Fr) (0.067 inches). According to some embodiments, the diameter of the side-hole of the endovascular device is at least 5.2 French (Fr) (0.068 inches). According to some embodiments, the diameter of the side-hole of the endovascular device is at least 5.3 French (Fr) (0.070 inches). According to some embodiments, the diameter of the side-hole of the endovascular device is at least 5.4 French (Fr) (0.071 inches). According to some embodiments, the diameter of the side-hole of the endovascular device is at least 5.5 French (Fr) (0.072 inches). According to some embodiments, the diameter of the side-hole of the endovascular device is at least 5.6 French (Fr) (0.073 inches). According to some embodiments, the diameter of the side-hole of the endovascular device is at least 5.7 French (Fr) (0.075 inches). According to some embodiments, the diameter of the side-hole of the endovascular device is at least 5.8 French (Fr) (0.076 inches). According to some embodiments, the diameter of the side-hole of the endovascular device is at least 5.9 French (Fr) (0.077 inches). According to some embodiments, the diameter of the side-hole of the endovascular device is at least 6 French (Fr) (0.079 inches). According to some embodiments, the diameter of the side-hole of the endovascular device is at least 6.1 French (Fr) (0.080 inches). According to some embodiments, the diameter of the side-hole of the endovascular device is at least 6.2 French (Fr) (0.081 inches). According to some embodiments, the diameter of the side-hole of the endovascular device is at least 6.3 French (Fr) (0.083 inches). According to some embodiments, the diameter of the side-hole of the endovascular device is at least 6.4 French (Fr) (0.084 inches). According to some embodiments, the diameter of the side-hole of the endovascular device is at least 6.5 French (Fr) (0.085 inches). According to some embodiments, the diameter of the side-hole of the endovascular device is at least 6.6 French (Fr) (0.087 inches). According to some embodiments, the diameter of the side-hole of the endovascular device is at least 6.7 French (Fr) (0.088 inches). According to some embodiments, the diameter of the side-hole of the endovascular device is at least 6.8 French (Fr) (0.089 inches). According to some embodiments, the diameter of the side-hole of the endovascular device is at least 6.9 French (Fr) (0.091 inches). According to some embodiments, the diameter of the side-hole of the endovascular device is at least 7 French (Fr) (0.092 inches). According to some embodiments, the diameter of the side-hole of the endovascular device is at least 7.1 French (Fr). According to some embodiments, the diameter of the side-hole of the endovascular device is at least 7.2 French (Fr). According to some embodiments, the diameter of the side-hole of the endovascular device is at least 7.3 French (Fr). According to some embodiments, the diameter of the side-hole of the endovascular device is at least 7.4 French (Fr). According to some embodiments, the diameter of the side-hole of the endovascular device is at least 7.5 French (Fr). According to some embodiments, the diameter of the side-hole of the endovascular device is at least 7.6 French (Fr). According to some embodiments, the diameter of the side-hole of the endovascular device is at least 7.7 French (Fr). According to some embodiments, the diameter of the side-hole of the endovascular device is at least 7.8 French (Fr). According to some embodiments, the diameter of the side-hole of the endovascular device is at least 7.9 French (Fr). According to some embodiments, the diameter of the side-hole of the endovascular device is at least 8.0 French (Fr). According to some embodiments, the diameter of the side-hole of the endovascular device is at least 8.1 French (Fr). According to some embodiments, the diameter of the side-hole of the endovascular device is at least 8.2 French (Fr). According to some embodiments, the diameter of the side-hole of the endovascular device is at least 8.3 French (Fr). According to some embodiments, the diameter of the side-hole of the endovascular device is at least 8.4 French (Fr). According to some embodiments, the diameter of the side-hole of the endovascular device is at least 8.5 French (Fr). According to some embodiments, the diameter of the side-hole of the endovascular device is at least 8.6 French (Fr). According to some embodiments, the diameter of the side-hole of the endovascular device is at least 8.7 French (Fr). According to some embodiments, the diameter of the side-hole of the endovascular device is at least 8.8 French (Fr). According to some embodiments, the diameter of the side-hole of the endovascular device is at least 8.9 French (Fr). According to some embodiments, the diameter of the side-hole of the endovascular device is at least 9.0 French (Fr). According to some embodiments, the diameter of the side-hole of the endovascular device is at least 9.1 French (Fr). According to some embodiments, the diameter of the side-hole of the endovascular device is at least 9.2 French (Fr). According to some embodiments, the diameter of the side-hole of the endovascular device is at least 9.3 French (Fr). According to some embodiments, the diameter of the side-hole of the endovascular device is at least 9.4 French (Fr). According to some embodiments, the diameter of the side-hole of the endovascular device is at least 9.5 French (Fr). According to some embodiments, the diameter of the side-hole of the endovascular device is at least 9.6 French (Fr). According to some embodiments, the diameter of the side-hole of the endovascular device is at least 9.7 French (Fr). According to some embodiments, the diameter of the side-hole of the endovascular device is at least 9.8 French (Fr). According to some embodiments, the diameter of the side-hole of the endovascular device is at least 9.9 French (Fr). According to some embodiments, the diameter of the side-hole of the endovascular device is at least 10.0 French (Fr). According to some embodiments, the diameter of the side-hole of the endovascular device is at least 10.1 French (Fr). According to some embodiments, the diameter of the side-hole of the endovascular device is at least 10.2 French (Fr). According to some embodiments, the diameter of the side-hole of the endovascular device is at least 10.3 French (Fr). According to some embodiments, the diameter of the side-hole of the endovascular device is at least 10.4 French (Fr). According to some embodiments, the diameter of the side-hole of the endovascular device is at least 10.5 French (Fr). According to some embodiments, the diameter of the side-hole of the endovascular device is at least 10.6 French (Fr). According to some embodiments, the diameter of the side-hole of the endovascular device is at least 10.7 French (Fr). According to some embodiments, the diameter of the side-hole of the endovascular device is at least 10.8 French (Fr). According to some embodiments, the diameter of the side-hole of the endovascular device is at least 10.9 French (Fr). According to some embodiments, the diameter of the side-hole of the endovascular device is at least 11.0 French (Fr). According to some embodiments, the diameter of the side-hole of the endovascular device is at least 11.1 French (Fr). According to some embodiments, the diameter of the side-hole of the endovascular device is at least 11.2 French (Fr). According to some embodiments, the diameter of the side-hole of the endovascular device is at least 11.3 French (Fr). According to some embodiments, the diameter of the side-hole of the endovascular device is at least 11.4 French (Fr). According to some embodiments, the diameter of the side-hole of the endovascular device is at least 11.5 French (Fr). According to some embodiments, the diameter of the side-hole of the endovascular device is at least 11.6 French (Fr). According to some embodiments, the diameter of the side-hole of the endovascular device is at least 11.7 French (Fr). According to some embodiments, the diameter of the side-hole of the endovascular device is at least 11.8 French (Fr). According to some embodiments, the diameter of the side-hole of the endovascular device is at least 11.9 French (Fr). According to some embodiments, the diameter of the side-hole of the endovascular device is at least 12.0 French (Fr). According to some embodiments, the diameter of the side-hole of the endovascular device is at least 12.1 French (Fr). According to some embodiments, the diameter of the side-hole of the endovascular device is at least 12.2 French (Fr). According to some embodiments, the diameter of the side-hole of the endovascular device is at least 12.3 French (Fr). According to some embodiments, the diameter of the side-hole of the endovascular device is at least 12.4 French (Fr). According to some embodiments, the diameter of the side-hole of the endovascular device is at least 12.5 French (Fr). According to some embodiments, the diameter of the side-hole of the endovascular device is at least 12.6 French (Fr). According to some embodiments, the diameter of the side-hole of the endovascular device is at least 12.7 French (Fr). According to some embodiments, the diameter of the side-hole of the endovascular device is at least 12.8 French (Fr). According to some embodiments, the diameter of the side-hole of the endovascular device is at least 12.9 French (Fr). According to some embodiments, the diameter of the side-hole of the endovascular device is at least 13.0 French (Fr). According to some embodiments, the diameter of the side-hole of the endovascular device is at least 13.1 French (Fr). According to some embodiments, the diameter of the side-hole of the endovascular device is at least 13.2 French (Fr). According to some embodiments, the diameter of the side-hole of the endovascular device is at least 13.3 French (Fr). According to some embodiments, the diameter of the side-hole of the endovascular device is at least 13.4 French (Fr). According to some embodiments, the diameter of the side-hole of the endovascular device is at least 13.5 French (Fr). According to some embodiments, the diameter of the side-hole of the endovascular device is at least 13.6 French (Fr). According to some embodiments, the diameter of the side-hole of the endovascular device is at least 13.7 French (Fr). According to some embodiments, the diameter of the side-hole of the endovascular device is at least 13.8 French (Fr). According to some embodiments, the diameter of the side-hole of the endovascular device is at least 13.9 French (Fr). According to some embodiments, the diameter of the side-hole of the endovascular device is at least 14.0 French (Fr). According to some embodiments, the diameter of the side-hole of the endovascular device is at least 14.1 French (Fr). According to some embodiments, the diameter of the side-hole of the endovascular device is at least 14.2 French (Fr). According to some embodiments, the diameter of the side-hole of the endovascular device is at least 14.3 French (Fr). According to some embodiments, the diameter of the side-hole of the endovascular device is at least 14.4 French (Fr). According to some embodiments, the diameter of the side-hole of the endovascular device is at least 14.5 French (Fr). According to some embodiments, the diameter of the side-hole of the endovascular device is at least 14.6 French (Fr). According to some embodiments, the diameter of the side-hole of the endovascular device is at least 14.7 French (Fr). According to some embodiments, the diameter of the side-hole of the endovascular device is at least 14.8 French (Fr). According to some embodiments, the diameter of the side-hole of the endovascular device is at least 14.9 French (Fr). According to some embodiments, the diameter of the side-hole of the endovascular device is at least 15.0 French (Fr). According to some embodiments, the diameter of the side-hole of the endovascular device is at least 15.0 French (Fr). According to some embodiments, the diameter of the side-hole of the endovascular device is at least 15.1 French (Fr). According to some embodiments, the diameter of the side-hole of the endovascular device is at least 15.2 French (Fr). According to some embodiments, the diameter of the side-hole of the endovascular device is at least 15.3 French (Fr). According to some embodiments, the diameter of the side-hole of the endovascular device is at least 15.4 French (Fr). According to some embodiments, the diameter of the side-hole of the endovascular device is at least 15.5 French (Fr). According to some embodiments, the diameter of the side-hole of the endovascular device is at least 15.6 French (Fr). According to some embodiments, the diameter of the side-hole of the endovascular device is at least 15.7 French (Fr). According to some embodiments, the diameter of the side-hole of the endovascular device is at least 15.8 French (Fr). According to some embodiments, the diameter of the side-hole of the endovascular device is at least 15.9 French (Fr). According to some embodiments, the diameter of the side-hole of the endovascular device is at least 16.0 French (Fr). According to some embodiments, the diameter of the side-hole of the endovascular device is at least 16.1 French (Fr). According to some embodiments, the diameter of the side-hole of the endovascular device is at least 16.2 French (Fr). According to some embodiments, the diameter of the side-hole of the endovascular device is at least 16.3 French (Fr). According to some embodiments, the diameter of the side-hole of the endovascular device is at least 16.4 French (Fr). According to some embodiments, the diameter of the side-hole of the endovascular device is at least 16.5 French (Fr). According to some embodiments, the diameter of the side-hole of the endovascular device is at least 16.6 French (Fr). According to some embodiments, the diameter of the side-hole of the endovascular device is at least 16.7 French (Fr). According to some embodiments, the diameter of the side-hole of the endovascular device is at least 16.8 French (Fr). According to some embodiments, the diameter of the side-hole of the endovascular device is at least 16.9 French (Fr). According to some embodiments, the diameter of the side-hole of the endovascular device is at least 17.0 French (Fr). According to some embodiments, the diameter of the side-hole of the endovascular device is at least 17.1 French (Fr). According to some embodiments, the diameter of the side-hole of the endovascular device is at least 17.2 French (Fr). According to some embodiments, the diameter of the side-hole of the endovascular device is at least 17.3 French (Fr). According to some embodiments, the diameter of the side-hole of the endovascular device is at least 17.4 French (Fr). According to some embodiments, the diameter of the side-hole of the endovascular device is at least 17.5 French (Fr). According to some embodiments, the diameter of the side-hole of the endovascular device is at least 17.6 French (Fr). According to some embodiments, the diameter of the side-hole of the endovascular device is at least 17.7 French (Fr). According to some embodiments, the diameter of the side-hole of the endovascular device is at least 17.8 French (Fr). According to some embodiments, the diameter of the side-hole of the endovascular device is at least 17.9 French (Fr). According to some embodiments, the diameter of the side-hole of the endovascular device is at least 18.0 French (Fr). According to some embodiments, the diameter of the side-hole of the endovascular device is at least 18.1 French (Fr). According to some embodiments, the diameter of the side-hole of the endovascular device is at least 18.2 French (Fr). According to some embodiments, the diameter of the side-hole of the endovascular device is at least 18.3 French (Fr). According to some embodiments, the diameter of the side-hole of the endovascular device is at least 18.4 French (Fr). According to some embodiments, the diameter of the side-hole of the endovascular device is at least 18.5 French (Fr). According to some embodiments, the diameter of the side-hole of the endovascular device is at least 18.6 French (Fr). According to some embodiments, the diameter of the side-hole of the endovascular device is at least 18.7 French (Fr). According to some embodiments, the diameter of the side-hole of the endovascular device is at least 18.8 French (Fr). According to some embodiments, the diameter of the side-hole of the endovascular device is at least 18.9 French (Fr). According to some embodiments, the diameter of the side-hole of the endovascular device is at least 19.0 French (Fr). According to some embodiments, the diameter of the side-hole of the endovascular device is at least 19.1 French (Fr). According to some embodiments, the diameter of the side-hole of the endovascular device is at least 19.2 French (Fr). According to some embodiments, the diameter of the side-hole of the endovascular device is at least 19.3 French (Fr). According to some embodiments, the diameter of the side-hole of the endovascular device is at least 19.4 French (Fr). According to some embodiments, the diameter of the side-hole of the endovascular device is at least 19.5 French (Fr). According to some embodiments, the diameter of the side-hole of the endovascular device is at least 19.6 French (Fr). According to some embodiments, the diameter of the side-hole of the endovascular device is at least 19.6 French (Fr). According to some embodiments, the diameter of the side-hole of the endovascular device is at least 19.7 French (Fr). According to some embodiments, the diameter of the side-hole of the endovascular device is at least 19.8 French (Fr). According to some embodiments, the diameter of the side-hole of the endovascular device is at least 19.9 French (Fr). According to some embodiments, the diameter of the side-hole of the endovascular device is at least 20.0 French (Fr). According to some embodiments, the diameter of the side-hole of the endovascular device is at least 20.1 French (Fr). According to some embodiments, the diameter of the side-hole of the endovascular device is at least 20.2 French (Fr). According to some embodiments, the diameter of the side-hole of the endovascular device is at least 20.3 French (Fr). According to some embodiments, the diameter of the side-hole of the endovascular device is at least 20.4 French (Fr). According to some embodiments, the diameter of the side-hole of the endovascular device is at least 20.5 French (Fr). According to some embodiments, the diameter of the side-hole of the endovascular device is at least 20.6 French (Fr). According to some embodiments, the diameter of the side-hole of the endovascular device is at least 20.7 French (Fr). According to some embodiments, the diameter of the side-hole of the endovascular device is at least 20.8 French (Fr). According to some embodiments, the diameter of the side-hole of the endovascular device is at least 20.9 French (Fr). According to some embodiments, the diameter of the side-hole of the endovascular device is at least 21.0 French (Fr). According to some embodiments, the diameter of the side-hole of the endovascular device is at least 21.1 French (Fr). According to some embodiments, the diameter of the side-hole of the endovascular device is at least 21.2 French (Fr). According to some embodiments, the diameter of the side-hole of the endovascular device is at least 21.3 French (Fr). According to some embodiments, the diameter of the side-hole of the endovascular device is at least 21.4 French (Fr). According to some embodiments, the diameter of the side-hole of the endovascular device is at least 21.5 French (Fr). According to some embodiments, the diameter of the side-hole of the endovascular device is at least 21.6 French (Fr). According to some embodiments, the diameter of the side-hole of the endovascular device is at least 21.7 French (Fr). According to some embodiments, the diameter of the side-hole of the endovascular device is at least 21.8 French (Fr). According to some embodiments, the diameter of the side-hole of the endovascular device is at least 21.9 French (Fr). According to some embodiments, the diameter of the side-hole of the endovascular device is at least 22.0 French (Fr). According to some embodiments, the diameter of the side-hole of the endovascular device is at least 22.1 French (Fr). According to some embodiments, the diameter of the side-hole of the endovascular device is at least 22.2 French (Fr). According to some embodiments, the diameter of the side-hole of the endovascular device is at least 22.3 French (Fr). According to some embodiments, the diameter of the side-hole of the endovascular device is at least 22.4 French (Fr). According to some embodiments, the diameter of the side-hole of the endovascular device is at least 22.5 French (Fr). According to some embodiments, the diameter of the side-hole of the endovascular device is at least 22.6 French (Fr). According to some embodiments, the diameter of the side-hole of the endovascular device is at least 22.7 French (Fr). According to some embodiments, the diameter of the side-hole of the endovascular device is at least 22.8 French (Fr). According to some embodiments, the diameter of the side-hole of the endovascular device is at least 22.9 French (Fr). According to some embodiments, the diameter of the side-hole of the endovascular device is at least 23.0 French (Fr). According to some embodiments, the diameter of the side-hole of the endovascular device is at least 23.1 French (Fr). According to some embodiments, the diameter of the side-hole of the endovascular device is at least 23.2 French (Fr). According to some embodiments, the diameter of the side-hole of the endovascular device is at least 23.3 French (Fr). According to some embodiments, the diameter of the side-hole of the endovascular device is at least 23.4 French (Fr). According to some embodiments, the diameter of the side-hole of the endovascular device is at least 23.5 French (Fr). According to some embodiments, the diameter of the side-hole of the endovascular device is at least 23.6 French (Fr). According to some embodiments, the diameter of the side-hole of the endovascular device is at least 23.7 French (Fr). According to some embodiments, the diameter of the side-hole of the endovascular device is at least 23.8 French (Fr). According to some embodiments, the diameter of the side-hole of the endovascular device is at least 23.9 French (Fr). According to some embodiments, the diameter of the side-hole of the endovascular device is at least 24.0 French (Fr). According to some embodiments, the diameter of the side-hole of the endovascular device is at least 24.1 French (Fr). According to some embodiments, the diameter of the side-hole of the endovascular device is at least 24.2 French (Fr). According to some embodiments, the diameter of the side-hole of the endovascular device is at least 24.3 French (Fr). According to some embodiments, the diameter of the side-hole of the endovascular device is at least 24.4 French (Fr). According to some embodiments, the diameter of the side-hole of the endovascular device is at least 24.5 French (Fr). According to some embodiments, the diameter of the side-hole of the endovascular device is at least 24.6 French (Fr). According to some embodiments, the diameter of the side-hole of the endovascular device is at least 24.7 French (Fr). According to some embodiments, the diameter of the side-hole of the endovascular device is at least 24.8 French (Fr). According to some embodiments, the diameter of the side-hole of the endovascular device is at least 24.9 French (Fr). According to some embodiments, the diameter of the side-hole of the endovascular device is at least 25.0 French (Fr). According to some embodiments, the diameter of the side-hole of the endovascular device is at least 25.0 French (Fr). According to some embodiments, the diameter of the side-hole of the endovascular device is at least 25.1 French (Fr). According to some embodiments, the diameter of the side-hole of the endovascular device is at least 5.2 French (Fr). According to some embodiments, the diameter of the side-hole of the endovascular device is at least 25.3 French (Fr). According to some embodiments, the diameter of the side-hole of the endovascular device is at least 25.4 French (Fr). According to some embodiments, the diameter of the side-hole of the endovascular device is at least 25.5 French (Fr). According to some embodiments, the diameter of the side-hole of the endovascular device is at least 25.6 French (Fr). According to some embodiments, the diameter of the side-hole of the endovascular device is at least 25.7 French (Fr). According to some embodiments, the diameter of the side-hole of the endovascular device is at least 25.8 French (Fr). According to some embodiments, the diameter of the side-hole of the endovascular device is at least 25.9 French (Fr). According to some embodiments, the diameter of the side-hole of the endovascular device is at least 26.0 French (Fr). According to some embodiments, the diameter of the side-hole of the endovascular device is at least 26.1 French (Fr). According to some embodiments, the diameter of the side-hole of the endovascular device is at least 26.2 French (Fr). According to some embodiments, the diameter of the side-hole of the endovascular device is at least 26.3 French (Fr). According to some embodiments, the diameter of the side-hole of the endovascular device is at least 26.4 French (Fr). According to some embodiments, the diameter of the side-hole of the endovascular device is at least 26.5 French (Fr). According to some embodiments, the diameter of the side-hole of the endovascular device is at least 26.6 French (Fr). According to some embodiments, the diameter of the side-hole of the endovascular device is at least 26.7 French (Fr). According to some embodiments, the diameter of the side-hole of the endovascular device is at least 26.8 French (Fr). According to some embodiments, the diameter of the side-hole of the endovascular device is at least 26.9 French (Fr). According to some embodiments, the diameter of the side-hole of the endovascular device is at least 27.0 French (Fr). According to some embodiments, the diameter of the side-hole of the endovascular device is at least 27.1 French (Fr). According to some embodiments, the diameter of the side-hole of the endovascular device is at least 27.2 French (Fr). According to some embodiments, the diameter of the side-hole of the endovascular device is at least 27.3 French (Fr). According to some embodiments, the diameter of the side-hole of the endovascular device is at least 27.4 French (Fr). According to some embodiments, the diameter of the side-hole of the endovascular device is at least 27.5 French (Fr). According to some embodiments, the diameter of the side-hole of the endovascular device is at least 27.6 French (Fr). According to some embodiments, the diameter of the side-hole of the endovascular device is at least 27.7 French (Fr). According to some embodiments, the diameter of the side-hole of the endovascular device is at least 27.8 French (Fr). According to some embodiments, the diameter of the side-hole of the endovascular device is at least 27.9 French (Fr). According to some embodiments, the diameter of the side-hole of the endovascular device is at least 28.0 French (Fr). According to some embodiments, the diameter of the side-hole of the endovascular device is at least 28.1 French (Fr). According to some embodiments, the diameter of the side-hole of the endovascular device is at least 28.2 French (Fr). According to some embodiments, the diameter of the side-hole of the endovascular device is at least 28.3 French (Fr). According to some embodiments, the diameter of the side-hole of the endovascular device is at least 28.4 French (Fr). According to some embodiments, the diameter of the side-hole of the endovascular device is at least 28.5 French (Fr). According to some embodiments, the diameter of the side-hole of the endovascular device is at least 28.6 French (Fr). According to some embodiments, the diameter of the side-hole of the endovascular device is at least 28.7 French (Fr). According to some embodiments, the diameter of the side-hole of the endovascular device is at least 28.8 French (Fr). According to some embodiments, the diameter of the side-hole of the endovascular device is at least 28.9 French (Fr). According to some embodiments, the diameter of the side-hole of the endovascular device is at least 29.0 French (Fr). According to some embodiments, the diameter of the side-hole of the endovascular device is at least 29.1 French (Fr). According to some embodiments, the diameter of the side-hole of the endovascular device is at least 29.2 French (Fr). According to some embodiments, the diameter of the side-hole of the endovascular device is at least 29.3 French (Fr). According to some embodiments, the diameter of the side-hole of the endovascular device is at least 29.4 French (Fr). According to some embodiments, the diameter of the side-hole of the endovascular device is at least 29.5 French (Fr). According to some embodiments, the diameter of the side-hole of the endovascular device is at least 29.6 French (Fr). According to some embodiments, the diameter of the side-hole of the endovascular device is at least 29.6 French (Fr). According to some embodiments, the diameter of the side-hole of the endovascular device is at least 29.7 French (Fr). According to some embodiments, the diameter of the side-hole of the endovascular device is at least 29.8 French (Fr). According to some embodiments, the diameter of the side-hole of the endovascular device is at least 29.9 French (Fr). According to some embodiments, the diameter of the side-hole of the endovascular device is at least 30.0 French (Fr).

According to some embodiments, the side-hole in side view is an ellipse comprising a length and a diameter. According to some embodiments, the side-hole of the endovascular device comprises a length. The length of the side-hole 310 is measured from the most proximal portion of the side-hole 310 to the most distal portion of the side-hole 310. According to some embodiments, the length of the side-hole of the endovascular device ranges from about 0.02 cm to at least 2 cm. According to some embodiments, the length of the side-hole of the endovascular device is at least 0.02 cm. According to some embodiments, the length of the side-hole of the endovascular device is at least 0.03 cm. According to some embodiments, the length of the side-hole of the endovascular device is at least 0.04 cm. According to some embodiments, the length of the side-hole of the endovascular device is at least 0.05 cm. According to some embodiments, the length of the side-hole of the endovascular device is at least 0.06 cm. According to some embodiments, the length of the side-hole of the endovascular device is at least 0.07 cm. According to some embodiments, the length of the side-hole of the endovascular device is at least 0.08 cm. According to some embodiments, the length of the side-hole of the endovascular device is at least 0.09 cm. According to some embodiments, the length of the side-hole of the endovascular device is at least 0.10 cm. According to some embodiments, the length of the side-hole of the endovascular device is at least 0.11 cm. According to some embodiments, the length of the side-hole of the endovascular device is at least 0.12 cm. According to some embodiments, the length of the side-hole of the endovascular device is at least 0.13 cm. According to some embodiments, the length of the side-hole of the endovascular device is at least 0.14 cm. According to some embodiments, the length of the side-hole of the endovascular device is at least 0.15 cm. According to some embodiments, the length of the side-hole of the endovascular device is at least 0.16 cm. According to some embodiments, the length of the side-hole of the endovascular device is at least 0.17 cm. According to some embodiments, the length of the side-hole of the endovascular device is at least 0.18 cm. According to some embodiments, the length of the side-hole of the endovascular device is at least 0.19 cm. According to some embodiments, the length of the side-hole of the endovascular device is at least 0.20 cm. According to some embodiments, the length of the side-hole of the endovascular device is at least 0.21 cm. According to some embodiments, the length of the side-hole of the endovascular device is at least 0.22 cm. According to some embodiments, the length of the side-hole of the endovascular device is at least 0.23 cm. According to some embodiments, the length of the side-hole of the endovascular device is at least 0.24 cm. According to some embodiments, the length of the side-hole of the endovascular device is at least 0.25 cm. According to some embodiments, the length of the side-hole of the endovascular device is at least 0.26 cm. According to some embodiments, the length of the side-hole of the endovascular device is at least 0.27 cm. According to some embodiments, the length of the side-hole of the endovascular device is at least 0.28 cm. According to some embodiments, the length of the side-hole of the endovascular device is at least 0.29 cm. According to some embodiments, the length of the side-hole of the endovascular device is at least 0.30 cm. According to some embodiments, the length of the side-hole of the endovascular device is at least 0.31 cm. According to some embodiments, the length of the side-hole of the endovascular device is at least 0.32 cm. According to some embodiments, the length of the side-hole of the endovascular device is at least 0.33 cm. According to some embodiments, the length of the side-hole of the endovascular device is at least 0.34 cm. According to some embodiments, the length of the side-hole of the endovascular device is at least 0.35 cm. According to some embodiments, the length of the side-hole of the endovascular device is at least 0.36 cm. According to some embodiments, the length of the side-hole of the endovascular device is at least 0.37 cm. According to some embodiments, the length of the side-hole of the endovascular device is at least 0.38 cm. According to some embodiments, the length of the side-hole of the endovascular device is at least 0.39 cm. According to some embodiments, the length of the side-hole of the endovascular device is at least 0.40 cm. According to some embodiments, the length of the side-hole of the endovascular device is at least 0.41 cm. According to some embodiments, the length of the side-hole of the endovascular device is at least 0.42 cm. According to some embodiments, the length of the side-hole of the endovascular device is at least 0.43 cm. According to some embodiments, the length of the side-hole of the endovascular device is at least 0.44 cm. According to some embodiments, the length of the side-hole of the endovascular device is at least 0.45 cm. According to some embodiments, the length of the side-hole of the endovascular device is at least 0.46 cm. According to some embodiments, the length of the side-hole of the endovascular device is at least 0.47 cm. According to some embodiments, the length of the side-hole of the endovascular device is at least 0.48 cm. According to some embodiments, the length of the side-hole of the endovascular device is at least 0.49 cm. According to some embodiments, the length of the side-hole of the endovascular device is at least 0.50 cm. According to some embodiments, the length of the side-hole of the endovascular device is at least 0.51 cm. According to some embodiments, the length of the side-hole of the endovascular device is at least 0.52 cm. According to some embodiments, the length of the side-hole of the endovascular device is at least 0.53 cm. According to some embodiments, the length of the side-hole of the endovascular device is at least 0.54 cm. According to some embodiments, the length of the side-hole of the endovascular device is at least 0.55 cm. According to some embodiments, the length of the side-hole of the endovascular device is at least 0.56 cm. According to some embodiments, the length of the side-hole of the endovascular device is at least 0.57 cm. According to some embodiments, the length of the side-hole of the endovascular device is at least 0.58 cm. According to some embodiments, the length of the side-hole of the endovascular device is at least 0.59 cm. According to some embodiments, the length of the side-hole of the endovascular device is at least 0.60 cm. According to some embodiments, the length of the side-hole of the endovascular device is at least 0.61 cm. According to some embodiments, the length of the side-hole of the endovascular device is at least 0.62 cm. According to some embodiments, the length of the side-hole of the endovascular device is at least 0.63 cm. According to some embodiments, the length of the side-hole of the endovascular device is at least 0.64 cm. According to some embodiments, the length of the side-hole of the endovascular device is at least 0.65 cm. According to some embodiments, the length of the side-hole of the endovascular device is at least 0.66 cm. According to some embodiments, the length of the side-hole of the endovascular device is at least 0.67 cm. According to some embodiments, the length of the side-hole of the endovascular device is at least 0.68 cm. According to some embodiments, the length of the side-hole of the endovascular device is at least 0.69 cm. According to some embodiments, the length of the side-hole of the endovascular device is at least 0.70 cm. According to some embodiments, the length of the side-hole of the endovascular device is at least 0.71 cm. According to some embodiments, the length of the side-hole of the endovascular device is at least 0.72 cm. According to some embodiments, the length of the side-hole of the endovascular device is at least 0.73 cm. According to some embodiments, the length of the side-hole of the endovascular device is at least 0.74 cm. According to some embodiments, the length of the side-hole of the endovascular device is at least 0.75 cm. According to some embodiments, the length of the side-hole of the endovascular device is at least 0.76 cm. According to some embodiments, the length of the side-hole of the endovascular device is at least 0.77 cm. According to some embodiments, the length of the side-hole of the endovascular device is at least 0.78 cm. According to some embodiments, the length of the side-hole of the endovascular device is at least 0.79 cm. According to some embodiments, the length of the side-hole of the endovascular device is at least 0.80 cm. According to some embodiments, the length of the side-hole of the endovascular device is at least 0.81 cm. According to some embodiments, the length of the side-hole of the endovascular device is at least 0.82 cm. According to some embodiments, the length of the side-hole of the endovascular device is at least 0.83 cm. According to some embodiments, the length of the side-hole of the endovascular device is at least 0.84 cm. According to some embodiments, the length of the side-hole of the endovascular device is at least 0.85 cm. According to some embodiments, the length of the side-hole of the endovascular device is at least 0.86 cm. According to some embodiments, the length of the side-hole of the endovascular device is at least 0.87 cm. According to some embodiments, the length of the side-hole of the endovascular device is at least 0.88 cm. According to some embodiments, the length of the side-hole of the endovascular device is at least 0.89 cm. According to some embodiments, the length of the side-hole of the endovascular device is at least 0.90 cm. According to some embodiments, the length of the side-hole of the endovascular device is at least 0.91 cm. According to some embodiments, the length of the side-hole of the endovascular device is at least 0.92 cm. According to some embodiments, the length of the side-hole of the endovascular device is at least 0.93 cm. According to some embodiments, the length of the side-hole of the endovascular device is at least 0.94 cm. According to some embodiments, the length of the side-hole of the endovascular device is at least 0.95 cm. According to some embodiments, the length of the side-hole of the endovascular device is at least 0.96 cm. According to some embodiments, the length of the side-hole of the endovascular device is at least 0.97 cm. According to some embodiments, the length of the side-hole of the endovascular device is at least 0.98 cm. According to some embodiments, the length of the side-hole of the endovascular device is at least 0.99 cm. According to some embodiments, the length of the side-hole of the endovascular device is at least 1 cm. According to some embodiments, the length of the side-hole of the endovascular device is at least 1.01 cm. According to some embodiments, the length of the side-hole of the endovascular device is at least 1.02 cm. According to some embodiments, the length of the side-hole of the endovascular device is at least 1.03 cm. According to some embodiments, the length of the side-hole of the endovascular device is at least 1.04 cm. According to some embodiments, the length of the side-hole of the endovascular device is at least 1.05 cm. According to some embodiments, the length of the side-hole of the endovascular device is at least 1.06 cm. According to some embodiments, the length of the side-hole of the endovascular device is at least 1.07 cm. According to some embodiments, the length of the side-hole of the endovascular device is at least 1.08 cm. According to some embodiments, the length of the side-hole of the endovascular device is at least 1.09 cm. According to some embodiments, the length of the side-hole of the endovascular device is at least 1.10 cm. According to some embodiments, the length of the side-hole of the endovascular device is at least 1.11 cm. According to some embodiments, the length of the side-hole of the endovascular device is at least 1.12 cm. According to some embodiments, the length of the side-hole of the endovascular device is at least 1.13 cm. According to some embodiments, the length of the side-hole of the endovascular device is at least 1.14 cm. According to some embodiments, the length of the side-hole of the endovascular device is at least 1.15 cm. According to some embodiments, the length of the side-hole of the endovascular device is at least 1.16 cm. According to some embodiments, the length of the side-hole of the endovascular device is at least 1.17 cm. According to some embodiments, the length of the side-hole of the endovascular device is at least 1.18 cm. According to some embodiments, the length of the side-hole of the endovascular device is at least 1.19 cm. According to some embodiments, the length of the side-hole of the endovascular device is at least 1.20 cm. According to some embodiments, the length of the side-hole of the endovascular device is at least 1.21 cm. According to some embodiments, the length of the side-hole of the endovascular device is at least 1.22 cm. According to some embodiments, the length of the side-hole of the endovascular device is at least 1.23 cm. According to some embodiments, the length of the side-hole of the endovascular device is at least 1.24 cm. According to some embodiments, the length of the side-hole of the endovascular device is at least 1.25 cm. According to some embodiments, the length of the side-hole of the endovascular device is at least 1.26 cm. According to some embodiments, the length of the side-hole of the endovascular device is at least 1.27 cm. According to some embodiments, the length of the side-hole of the endovascular device is at least 1.28 cm. According to some embodiments, the length of the side-hole of the endovascular device is at least 1.29 cm. According to some embodiments, the length of the side-hole of the endovascular device is at least 1.30 cm. According to some embodiments, the length of the side-hole of the endovascular device is at least 1.31 cm. According to some embodiments, the length of the side-hole of the endovascular device is at least 1.32 cm. According to some embodiments, the length of the side-hole of the endovascular device is at least 1.33 cm. According to some embodiments, the length of the side-hole of the endovascular device is at least 1.34 cm. According to some embodiments, the length of the side-hole of the endovascular device is at least 1.35 cm. According to some embodiments, the length of the side-hole of the endovascular device is at least 1.36 cm. According to some embodiments, the length of the side-hole of the endovascular device is at least 1.37 cm. According to some embodiments, the length of the side-hole of the endovascular device is at least 1.38 cm. According to some embodiments, the length of the side-hole of the endovascular device is at least 1.39 cm. According to some embodiments, the length of the side-hole of the endovascular device is at least 1.40 cm. According to some embodiments, the length of the side-hole of the endovascular device is at least 1.41 cm. According to some embodiments, the length of the side-hole of the endovascular device is at least 1.42 cm. According to some embodiments, the length of the side-hole of the endovascular device is at least 1.43 cm. According to some embodiments, the length of the side-hole of the endovascular device is at least 1.44 cm. According to some embodiments, the length of the side-hole of the endovascular device is at least 1.45 cm. According to some embodiments, the length of the side-hole of the endovascular device is at least 1.46 cm. According to some embodiments, the length of the side-hole of the endovascular device is at least 1.47 cm. According to some embodiments, the length of the side-hole of the endovascular device is at least 1.48 cm. According to some embodiments, the length of the side-hole of the endovascular device is at least 1.49 cm. According to some embodiments, the length of the side-hole of the endovascular device is at least 1.50 cm. According to some embodiments, the length of the side-hole of the endovascular device is at least 1.51 cm. According to some embodiments, the length of the side-hole of the endovascular device is at least 1.52 cm. According to some embodiments, the length of the side-hole of the endovascular device is at least 1.53 cm. According to some embodiments, the length of the side-hole of the endovascular device is at least 1.54 cm. According to some embodiments, the length of the side-hole of the endovascular device is at least 1.55 cm. According to some embodiments, the length of the side-hole of the endovascular device is at least 1.56 cm. According to some embodiments, the length of the side-hole of the endovascular device is at least 1.57 cm. According to some embodiments, the length of the side-hole of the endovascular device is at least 1.58 cm. According to some embodiments, the length of the side-hole of the endovascular device is at least 1.59 cm. According to some embodiments, the length of the side-hole of the endovascular device is at least 1.60 cm. According to some embodiments, the length of the side-hole of the endovascular device is at least 1.61 cm. According to some embodiments, the length of the side-hole of the endovascular device is at least 1.62 cm. According to some embodiments, the length of the side-hole of the endovascular device is at least 1.63 cm. According to some embodiments, the length of the side-hole of the endovascular device is at least 1.64 cm. According to some embodiments, the length of the side-hole of the endovascular device is at least 1.65 cm. According to some embodiments, the length of the side-hole of the endovascular device is at least 1.66 cm. According to some embodiments, the length of the side-hole of the endovascular device is at least 1.67 cm. According to some embodiments, the length of the side-hole of the endovascular device is at least 1.68 cm. According to some embodiments, the length of the side-hole of the endovascular device is at least 1.69 cm. According to some embodiments, the length of the side-hole of the endovascular device is at least 1.70 cm. According to some embodiments, the length of the side-hole of the endovascular device is at least 1.71 cm. According to some embodiments, the length of the side-hole of the endovascular device is at least 1.72 cm. According to some embodiments, the length of the side-hole of the endovascular device is at least 1.73 cm. According to some embodiments, the length of the side-hole of the endovascular device is at least 1.74 cm. According to some embodiments, the length of the side-hole of the endovascular device is at least 1.75 cm. According to some embodiments, the length of the side-hole of the endovascular device is at least 1.76 cm. According to some embodiments, the length of the side-hole of the endovascular device is at least 1.77 cm. According to some embodiments, the length of the side-hole of the endovascular device is at least 1.78 cm. According to some embodiments, the length of the side-hole of the endovascular device is at least 1.79 cm. According to some embodiments, the length of the side-hole of the endovascular device is at least 1.80 cm. According to some embodiments, the length of the side-hole of the endovascular device is at least 1.81 cm. According to some embodiments, the length of the side-hole of the endovascular device is at least 1.82 cm. According to some embodiments, the length of the side-hole of the endovascular device is at least 1.83 cm. According to some embodiments, the length of the side-hole of the endovascular device is at least 1.84 cm. According to some embodiments, the length of the side-hole of the endovascular device is at least 1.85 cm. According to some embodiments, the length of the side-hole of the endovascular device is at least 1.86 cm. According to some embodiments, the length of the side-hole of the endovascular device is at least 1.87 cm. According to some embodiments, the length of the side-hole of the endovascular device is at least 1.88 cm. According to some embodiments, the length of the side-hole of the endovascular device is at least 1.89 cm. According to some embodiments, the length of the side-hole of the endovascular device is at least 1.90 cm. According to some embodiments, the length of the side-hole of the endovascular device is at least 1.91 cm. According to some embodiments, the length of the side-hole of the endovascular device is at least 1.92 cm. According to some embodiments, the length of the side-hole of the endovascular device is at least 1.93 cm. According to some embodiments, the length of the side-hole of the endovascular device is at least 1.94 cm. According to some embodiments, the length of the side-hole of the endovascular device is at least 1.95 cm. According to some embodiments, the length of the side-hole of the endovascular device is at least 1.96 cm. According to some embodiments, the length of the side-hole of the endovascular device is at least 1.97 cm. According to some embodiments, the length of the side-hole of the endovascular device is at least 1.98 cm. According to some embodiments, the length of the side-hole of the endovascular device is at least 1.99 cm. According to some embodiments, the length of the side-hole of the endovascular device is at least 2 cm.

As described above, according to some embodiments, part of or all of the first segment/working segment and/or the second segment/support segment of each endovascular device can be preformed in form of a geometric shape. Exemplary geometric shapes of the support segment can include, without limitation, a “J” shape, a “C” shape and an “S” shape. According to some embodiments, the shape of part of or all of the support segment is a pre-shaped configuration. According to some embodiments, the shape of the support segment is inserted into the body of a subject in a straight configuration and is subsequently re-shaped into a pre-shaped geometric configuration. According to some embodiments, the shape of the support segment is adjustable. According to some embodiments, the support segment can be straightened by, for example, a dilator, a stylet, a wire, etc. for introduction into the body of a subject. According to some embodiments, the support segment is achieved by steam shaping. According to some embodiments the steam shaping is effective to re-shape the support segment of the device.

According to some embodiments, the shape of the support segment comprises, without limitation, a shape memory polymer (SMP), a shape memory alloy (SMA) or a combination thereof.

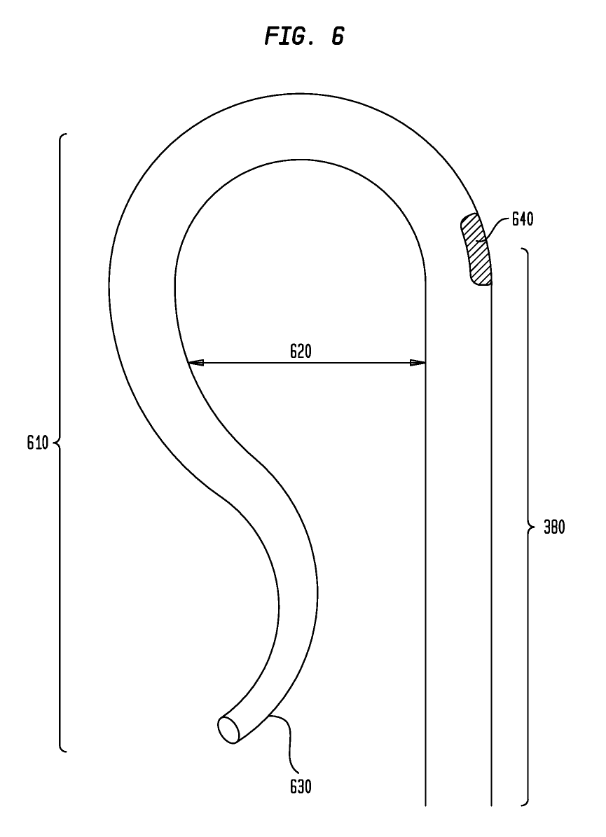

According to some embodiments, the shape of at least part of the support segment is an “S” shape. According to some embodiments, the “S” shape is a shepherd's hook shape. For example, FIG. 6 shows an exemplary and non-limiting example of some embodiments of “S”-shaped support segment 610.

According to some embodiments, the “S”-shaped support segment 610 can be used to access difficult innominate arteries; to subsequently access the right common carotid artery and its distal branches; and/or to subsequently access the right subclavian artery and/or its branches. By way of non-limiting example, in a subject with an overgrown, tortuous and/or long aortic arch, and/or an elongated, straightened and/or tortuous innominate artery, the “S”-shaped support segment 610 can be used to distally access the right subclavian artery and right common carotid artery. According to some embodiments, the “S”-shaped support segment 610 can be used at least at the intracranial carotid terminus.

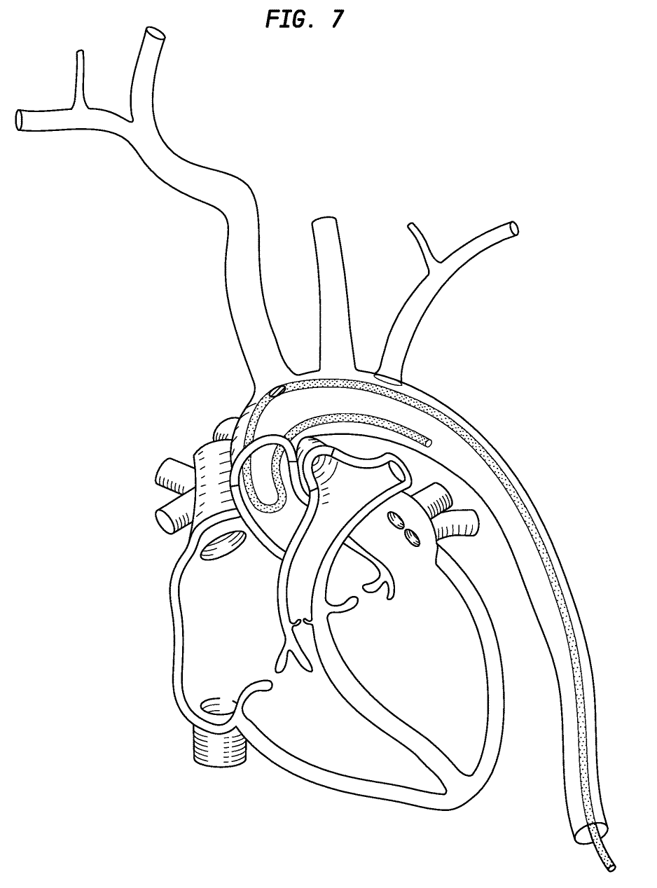

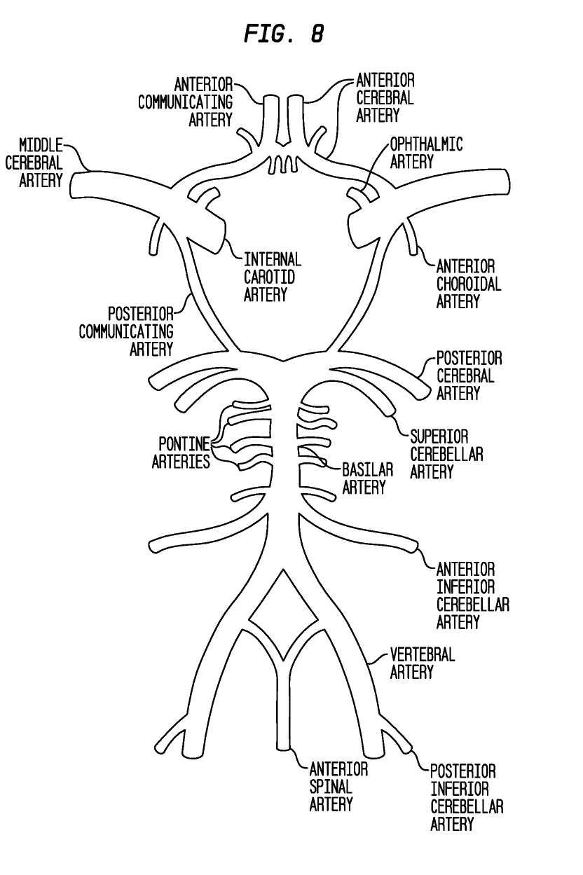

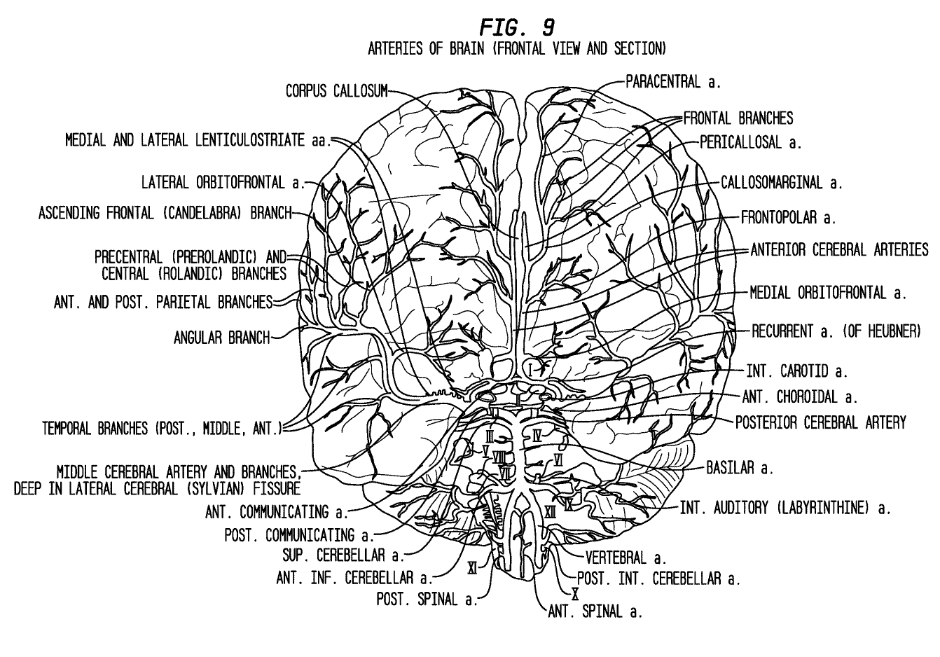

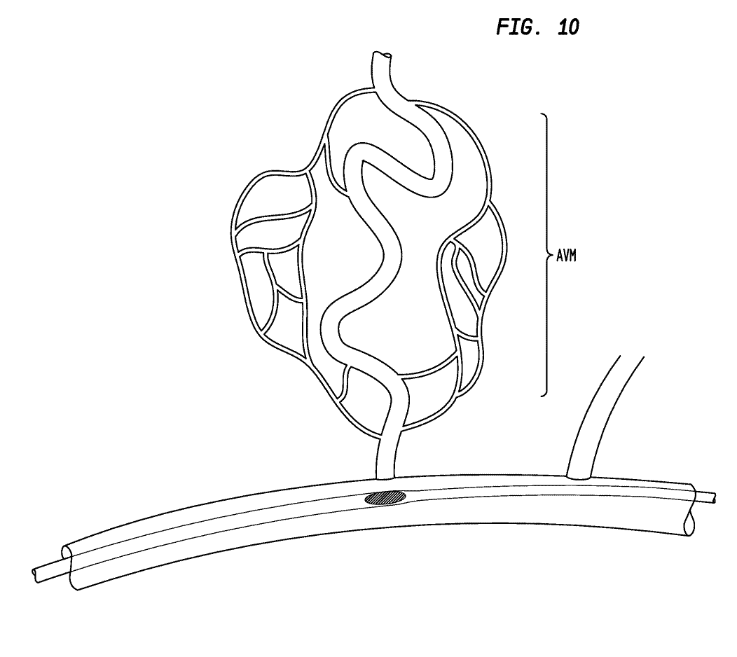

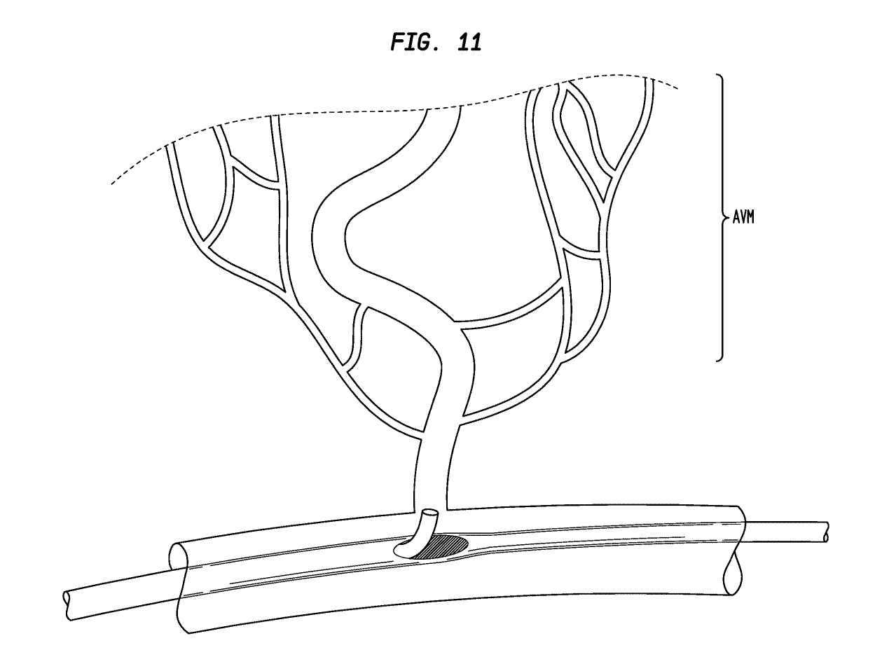

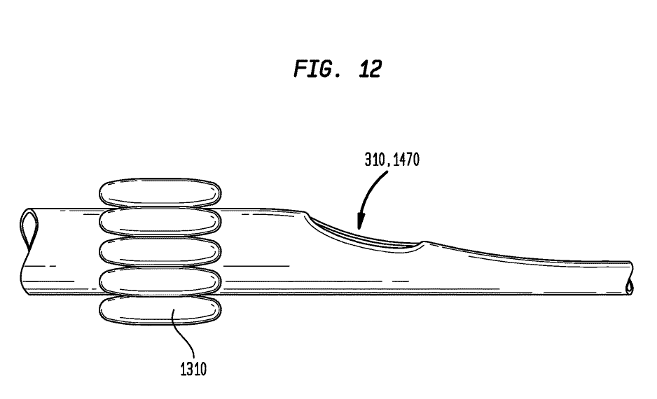

According to some embodiments, the “S” shape is a pre-shaped configuration. According to some embodiments, the “S”-shaped support segment 610 is inserted into the body of a subject in a straight configuration and is subsequently re-shaped into a pre-shaped “S” configuration. According to some embodiments, the “S”-shaped support segment 610 is inserted into the body of a subject in a straight configuration, and , as depicted in FIG. 7, e.g., into the aortic arch (see FIG. 7) in a straight configuration, and the distal portion 630 of the “S”-shaped support segment 610 curves back across the aortic arch, (see FIG. 7), which is effective to provide support for the endovascular device, to facilitate placement of the endovascular device 100, to anchor the endovascular device within a blood vessel, to prevent kickback of the endovascular device 100, or a combination thereof (FIG. 7). According to some embodiments, the “S”-shaped support segment 610 is inserted into the body of a subject in a straight configuration, into the aortic arch in a straight configuration such that , and the distal portion 630 of the “S”-shaped support segment 610 curves back across the aortic arch and down the descending aorta to provide support for the endovascular device, to facilitate placement of the endovascular device, to anchor the endovascular device within a blood vessel, to prevent kickback of the endovascular device, or a combination thereof (FIG. 7). According to some embodiments, the placement of the endovascular device is with the primary opening positioned at the origin of the innominate artery.

According to some embodiments, the geometrically shaped support segment comprises a shapeable material. Exemplary shapeable materials include, without limitation, a shape memory polymer (SMP), a shape memory alloy (SMA) or a combination thereof.

According to some embodiments, the geometrically shaped support segment 610 comprises a shape memory polymer (SMP). Shape memory polymers include, but are not limited to methacrylates, polyurethanes, blends of polystyrene and polyurethane, and polyvinylchloride.

According to some embodiments, the geometrically shaped support segment 610 comprises a shape memory alloy (SMA). Non-limiting examples of shape memory alloys include nickel-titanium (i.e., nitinol).

According to some embodiments, the length of the “S”-shaped support segment 610 is at least 2% longer than the length of the working segment. According to some embodiments, the length of the “S”-shaped support segment 610 is at least 2.5% longer than the length of the working segment. According to some embodiments, the length of “S”-shaped support segment 610 is at least 3% longer than the length of the working segment. According to some embodiments, the length of “S”-shaped support segment 610 is at least 3.5% longer than the length of working segment. According to some embodiments, the length of “S”-shaped support segment 610 is at least 4% longer than the length of the working segment. According to some embodiments, the length of “S”-shaped support segment 610 is at least 4.5% longer than the length of the working segment. According to some embodiments, the length of “S”-shaped support segment 610 is at least 5% longer than the length of the the working segment. According to some embodiments, the length of the “S”-shaped support segment 610 is at least 5.5% longer than the length of the working segment. According to some embodiments, the length of the “S”-shaped support segment 610 is at least 6% longer than the length of the working segment. According to some embodiments, the length of the “S”-shaped support segment 610 is at least 6.5% longer than the length of the working segment. According to some embodiments, the length of the “S”-shaped support segment 610 is at least 7% longer than the length of the working segment. According to some embodiments, the length of the “S”-shaped support segment 610 is at least 7.5% longer than the length of the working segment. According to some embodiments, the length of the “S”-shaped support segment 610 is at least 8% longer than the length of the working segment. According to some embodiments, the length of the “S”-shaped support segment 610 is at least 8.5% longer than the length of the working segment. According to some embodiments, the length of the “S”-shaped support segment 610 is at least 9% longer than the length of the working segment. According to some embodiments, the length of the “S”-shaped support segment 610 is at least 9.5% longer than the length of the working segment. According to some embodiments, the length of the “S”-shaped support segment 610 is at least 10% longer than the length of the working segment. According to some embodiments, the length of the “S”-shaped support segment 610 is at least 20% longer than the length of the working segment. According to some embodiments, the length of the “S”-shaped support segment 610 is at least 30% longer than the length of the working segment. According to some embodiments, the length of the “S”-shaped support segment 610 is at least 40% longer than the length of the working segment. According to some embodiments, the length of the “S”-shaped support segment 610 is at least 50% longer than the length of the working segment. According to some embodiments, the length of the “S”-shaped support segment 610 is at least 60% longer than the length of the working segment. According to some embodiments, the length of the “S”-shaped support segment 610 is at least 70% longer than the length of the working segment. According to some embodiments, the length of the “S”-shaped support segment 610 is at least 80% longer than the length of the working segment. According to some embodiments, the length of the “S”-shaped support segment 610 is at least 90% longer than the length of the working segment. According to some embodiments, the length of the “S”-shaped support segment 610 is at least 100% longer than the length of the working segment. According to some embodiments, the length of the “S”-shaped support segment 610 is at least 110% longer than the length of the working segment. According to some embodiments, the length of the “S”-shaped support segment 610 is at least 380% longer than the length of the working segment. According to some embodiments, the length of the “S”-shaped support segment 610 is at least 130% longer than the length of the working segment. According to some embodiments, the length of the “S”-shaped support segment 610 is at least 140% longer than the length of the working segment. According to some embodiments, the length of the “S”-shaped support segment 610 is at least 150% longer than the length of the working segment. According to some embodiments, the length of the “S”-shaped support segment 610 is at least 160% longer than the length of the working segment. According to some embodiments, the length of the “S”-shaped support segment 610 is at least 170% longer than the length of the working segment. According to some embodiments, the length of the “S”-shaped support segment 610 is at least 180% longer than the length of the working segment. According to some embodiments, the length of the “S”-shaped support segment 610 is at least 190% longer than the length of the working segment. According to some embodiments, the length of the “S”-shaped support segment 610 is at least 200% longer than the length of the working segment. According to some embodiments, the length of the “S”-shaped support segment 610 is at least 210% longer than the length of the working segment. According to some embodiments, the length of the “S”-shaped support segment 610 is at least 220% longer than the length of the working segment. According to some embodiments, the length of the “S”-shaped support segment 610 is at least 230% longer than the length of the working segment. According to some embodiments, the length of the “S”-shaped support segment 610 is at least 240% longer than the length of the working segment. According to some embodiments, the length of the “S”-shaped support segment 610 is at least 250% longer than the length of the working segment. According to some embodiments, the length of the “S”-shaped support segment 610 is at least 260% longer than the length of the working segment. According to some embodiments, the length of the “S”-shaped support segment 610 is at least 270% longer than the length of the working segment. According to some embodiments, the length of the “S”-shaped support segment 610 is at least 280% longer than the length of the working segment. According to some embodiments, the length of the “S”-shaped support segment 610 is at least 290% longer than the length of the working segment. According to some embodiments, the length of the “S”-shaped support segment 610 is at least 300% longer than the length of the working segment.

According to some embodiments, “S”-shaped support segment 610 comprises a curve diameter 620. According to some embodiments, curve diameter 620 ranges from about 1 mm to about 10 cm. According to some embodiments, curve diameter 620 ranges from about 1 mm to at least 10 cm. According to some embodiments, curve diameter 620 ranges from at least 1 mm to at least 10 cm. According to some embodiments, curve diameter 620 is at least 1 mm. According to some embodiments, curve diameter 620 is at least 2 mm. According to some embodiments, curve diameter 620 is at least 3 mm. According to some embodiments, curve diameter 620is at least 4 mm. According to some embodiments, curve diameter 620 is at least 5 mm. According to some embodiments, curve diameter 620 is at least 6 mm. According to some embodiments, curve diameter 620 is at least 7 mm. According to some embodiments, curve diameter 620 is at least 8 mm. According to some embodiments, curve diameter 620 is at least 9 mm. According to some embodiments, curve diameter 620 is at least 1 cm. According to some embodiments, curve diameter 620 is at least 2 cm. According to some embodiments, curve diameter 620 is at least 3 cm. According to some embodiments, curve diameter 620 is at least 4 cm. According to some embodiments, curve diameter 620 is at least 5 cm. According to some embodiments, curve diameter 620 is at least 6 cm. According to some embodiments, curve diameter 620 is at least 7 cm. According to some embodiments, curve diameter 620 is at least 8 cm. According to some embodiments, curve diameter 620 is at least 9 cm. According to some embodiments, curve diameter 620 is at least 10 cm.

According to some embodiments, a luer lock is attached to the proximal end of the working segment, to the proximal end of the support segment, or both.

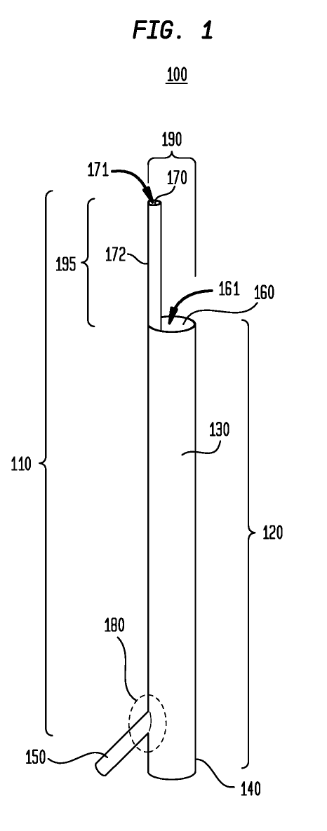

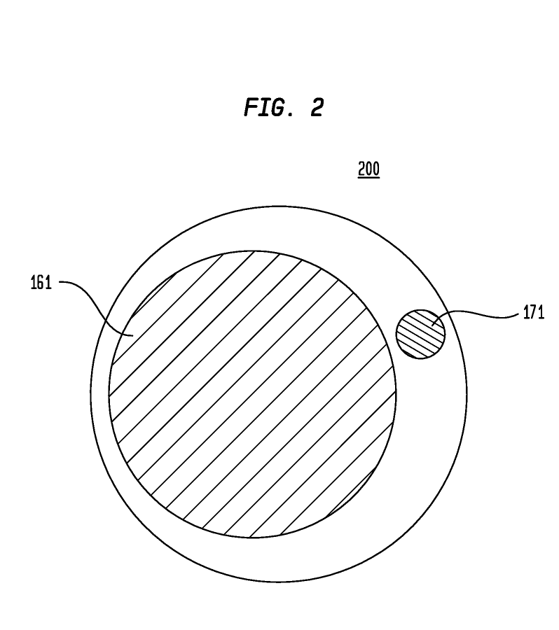

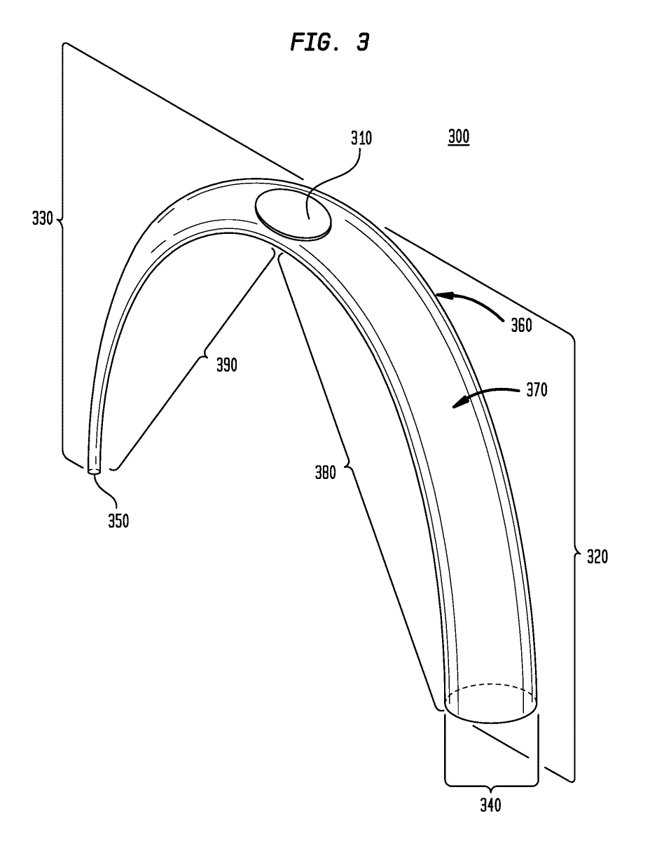

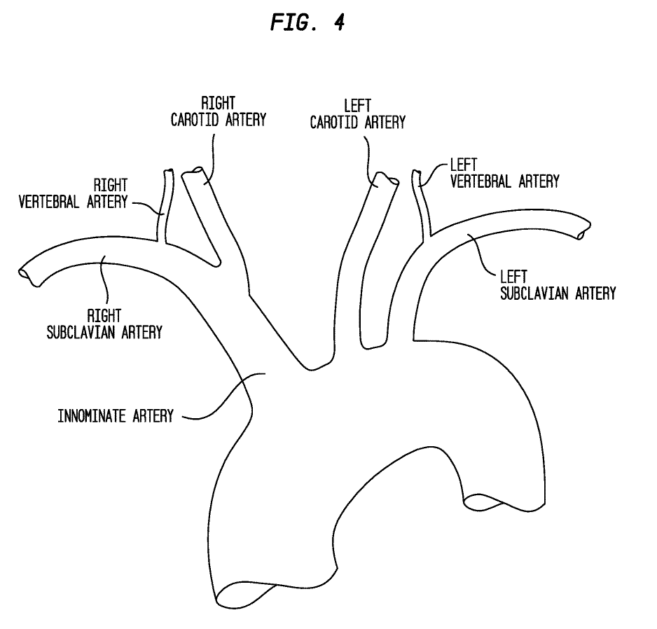

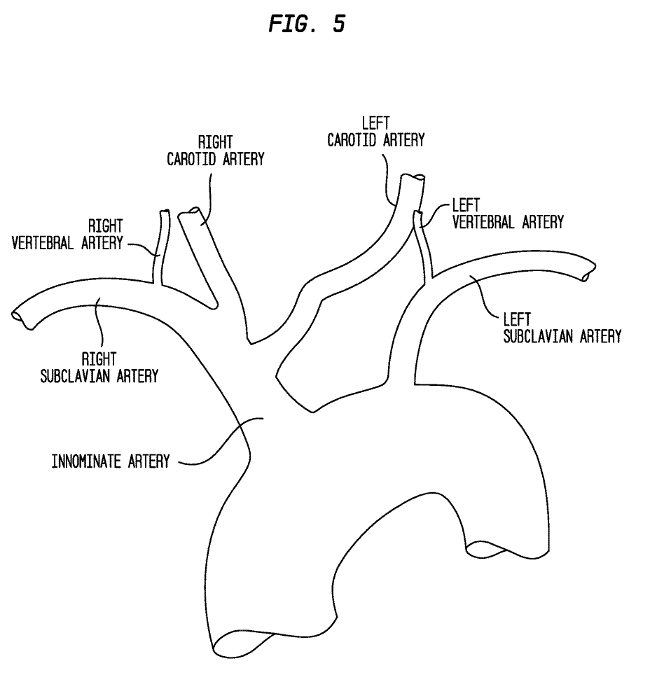

### The Exemplary Catheter of FIGS. 1 and 2

FIGS. 1 and 2 show an exemplary and non-limiting example of some embodiments of the endovascular device 100 of the described invention. According to one possible configuration, FIG. 1 illustrates a side view of the endovascular device 100 comprising a tube 130 defining a working lumen 161. According to some embodiments, tube 130 further comprises a support tube 172 defining a support lumen 171. According to some embodiments, tube 130 comprises a bifurcation 180 at a first end and openings 190 at a second end. The bifurcation comprises a first branch 140 and a second branch 150. The openings comprise a primary opening 160 leading to the working lumen 161 and a secondary opening 170 leading to the support lumen 171. The first branch 140, the working lumen 161, and the primary opening 160 form a working segment 120, which contains both the entirety of the working lumen 161 and the proximal portion of the support lumen 171. A segment 110 incorporates the entirety of the device. The part of the support tube 172 distal to the primary opening 160 comprises the distal support segment 195, which incorporates the distal segment of the support lumen 171 and the secondary opening 170. According to some embodiments, a luer lock is attached to the proximal end (first end) of each branch. FIG. 2 illustrates a cross-sectional view 200 through segment 120 of the endovascular device 100 comprising a cross-sectional view of the working lumen 161 and a cross-sectional view of the support lumen 171 at the middle of working segment 120 of the device.

According to some embodiments, the distal support segment 195 and or the working segment 120 is rigid. According to some embodiments, the distal support segment 195 and/or the working segment 120 comprises a soft, flexible portion.

According to some embodiments, the endovascular device 100 is an intracranial endovascular device. According to some embodiments, the endovascular device is a peripheral blood vessel endovascular device. According to some embodiments, the endovascular device is a cardiac blood vessel endovascular device.

According to some embodiments, the length of the working segment 120 of the endovascular device ranges from about 20 cm to about 160 cm. According to some embodiments, the length of the working segment 120 of the endovascular device ranges from at least 20 cm to at least 160 cm. According to some embodiments, the length of the working segment 120 of the endovascular device is about 20 cm. According to some embodiments, the length of the working segment 120 of the endovascular device is about 25 cm. According to some embodiments, the length of the working segment 120 of the endovascular device is about 30 cm. According to some embodiments, the length of the working segment 120 of the endovascular device is about 35 cm. According to some embodiments, the length of the working segment 120 of the endovascular device is about 40 cm. According to some embodiments, the length of the working segment 120 of the endovascular device is about 45 cm. According to some embodiments, the length of the working segment 120 of the endovascular device is about 50 cm. According to some embodiments, the length of the working segment 120 of the endovascular device is about 55 cm. According to some embodiments, the length of the working segment 120 of the endovascular device is about 60 cm. According to some embodiments, the length of the working segment 120 of the endovascular device is at least 65 cm. According to some embodiments, the length of the working segment 120 of the endovascular device is at least 70 cm. According to some embodiments, the length of the working segment 120 of the endovascular device is at least 75 cm. According to some embodiments, the length of the working segment 120 of the endovascular device is at least 80 cm. According to some embodiments, the length of the working segment 120 of the endovascular device is at least 85 cm. According to some embodiments, the length of the working segment 120 of the endovascular device is at least 90 cm. According to some embodiments, the length of the working segment 120 of the endovascular device is at least 95 cm. According to some embodiments, the length of the working segment 120 of the endovascular device is at least 100 cm. According to some embodiments, the length of the working segment 120 of the endovascular device is at least 105 cm. According to some embodiments, the length of the working segment 120 of the endovascular device is at least 110 cm. According to some embodiments, the length of the working segment 120 of the endovascular device is at least 115 cm. According to some embodiments, the length of the working segment 120 of the endovascular device is at least 120 cm. According to some embodiments, the length of the working segment 120 of the endovascular device is at least 125 cm. According to some embodiments, the length of the working segment 120 of the endovascular device is at least 130 cm. According to some embodiments, the length of the working segment 120 of the endovascular device is at least 135 cm. According to some embodiments, the length of the working segment 120 of the endovascular device is at least 140 cm. According to some embodiments, the length of the working segment 120 of the endovascular device is at least 145 cm. According to some embodiments, the length of the working segment 120 of the endovascular device is at least 150 cm. According to some embodiments, the length of the working segment 120 of the endovascular device is at least 155 cm. According to some embodiments, the length of the working segment 120 of the endovascular device is at least 160 cm.

According to some embodiments, the inner diameter (ID) of the working lumen 161 of the endovascular device ranges from about 0.1 French (Fr) (0.001 inches) to about 30 French (Fr) (0.394 inches). According to some embodiments, the inner diameter (ID) of the working lumen 161 of the endovascular device ranges from about 0.1 French (Fr) (0.001 inches) to at least 30 French (Fr) (0.394 inches). According to some embodiments, the inner diameter (ID) of the working lumen 161 of the endovascular device ranges from at least 0.1 French (Fr) (0.001 inches) to at least 30 French (Fr) (0.394 inches). According to some embodiments, the inner diameter (ID) of the working lumen 161 of the endovascular device is at least 0.1 French (Fr) (0.001 inches). According to some embodiments, the inner diameter (ID) of the working lumen 161 of the endovascular device is at least 0.2 French (Fr) (0.002 inches). According to some embodiments, the inner diameter (ID) of the working lumen 161 of the endovascular device is at least 0.3 French (Fr) (0.003 inches). According to some embodiments, the inner diameter (ID) of the working segment 120 of the endovascular device is at least 0.4 French (Fr) (0.005 inches). According to some embodiments, the inner diameter (ID) of the working lumen 161 of the endovascular device is at least 0.5 French (Fr) (0.007 inches). According to some embodiments, the inner diameter (ID) of the working lumen 161 of the endovascular device is at least 0.6 French (Fr) (0.008 inches). According to some embodiments, the inner diameter (ID) of the working lumen 161 of the endovascular device is at least 0.7 French (Fr) (0.009 inches). According to some embodiments, the inner diameter (ID) of the working lumen 161 of the endovascular device is at least 0.8 French (Fr) (0.01 inches). According to some embodiments, the inner diameter (ID) of the working lumen 161 of the endovascular device is at least 0.9 French (Fr) (0.012 inches). According to some embodiments, the inner diameter (ID) of the working lumen 161 of the endovascular device is at least 1 French (Fr) (0.013 inches). According to some embodiments, the inner diameter (ID) of the working lumen 161 of the endovascular device is at least 2 French (Fr) (0.026 inches). According to some embodiments, the inner diameter (ID) of the working lumen 161 of the endovascular device is at least 3 French (Fr) (0.039 inches). According to some embodiments, the inner diameter (ID) of the working lumen 161 of the endovascular device is at least 4 French (Fr) (0.052 inches). According to some embodiments, the inner diameter (ID) of the working lumen 161 of the endovascular device is at least 5 French (Fr) (0.066 inches). According to some embodiments, the inner diameter (ID) of the working lumen 161 of the endovascular device is at least 6 French (Fr) (0.079 inches). According to some embodiments, the inner diameter (ID) of the working lumen 161 of the endovascular device is at least 7 French (Fr) (0.092 inches). According to some embodiments, the inner diameter (ID) of the working lumen 161 of the endovascular device is at least 8 French (Fr) (0.105 inches). According to some embodiments, the inner diameter (ID) of the working lumen 161 of the endovascular device is at least 9 French (Fr) (0.118 inches). According to some embodiments, the inner diameter (ID) of the working lumen 161 of the endovascular device is at least 10 French (Fr) (0.131 inches). According to some embodiments, the inner diameter (ID) of the working lumen 161 of the endovascular device is at least 11 French (Fr) (0.144 inches). According to some embodiments, the inner diameter (ID) of the working lumen 161 of the endovascular device is at least 12 French (Fr) (0.157 inches). According to some embodiments, the inner diameter (ID) of the working lumen 161 of the endovascular device is at least 13 French (Fr) (0.171 inches). According to some embodiments, the inner diameter (ID) of the working lumen 161 of the endovascular device is at least 14 French (Fr) (0.184 inches). According to some embodiments, the inner diameter (ID) of the working lumen 161 of the endovascular device is at least 15 French (Fr) (0.197 inches). According to some embodiments, the inner diameter (ID) of the working lumen 161 of the endovascular device is at least 16 French (Fr) (0.210 inches). According to some embodiments, the inner diameter (ID) of the working lumen 161 of the endovascular device is at least 17 French (Fr) (0.223 inches). According to some embodiments, the inner diameter (ID) of the working lumen 161 of the endovascular device is at least 18 French (Fr) (0.236 inches). According to some embodiments, the inner diameter (ID) of the working lumen 161 of the endovascular device is at least 19 French (Fr) (0.249 inches). According to some embodiments, the inner diameter (ID) of the working lumen 161 of the endovascular device is at least 20 French (Fr) (0.262 inches). According to some embodiments, the inner diameter (ID) of the working lumen 161 of the endovascular device is at least 21 French (Fr) (0.276 inches). According to some embodiments, the inner diameter (ID) of the working lumen 161 of the endovascular device is at least 22 French (Fr) (0.289 inches). According to some embodiments, the inner diameter (ID) of the working lumen 161 of the endovascular device is at least 23 French (Fr) (0.302 inches). According to some embodiments, the inner diameter (ID) of the working lumen 161 of the endovascular device is at least 24 French (Fr) (0.315 inches). According to some embodiments, the inner diameter (ID) of the working lumen 161 of the endovascular device is at least 25 French (Fr) (0.328 inches). According to some embodiments, the inner diameter (ID) of the working lumen 161 of the endovascular device is at least 26 French (Fr) (0.341 inches). According to some embodiments, the inner diameter (ID) of the working lumen 161 of the endovascular device is at least 27 French (Fr) (0.354 inches). According to some embodiments, the inner diameter (ID) of the working lumen 161 of the endovascular device is at least 28 French (Fr) (0.367 inches). According to some embodiments, the inner diameter (ID) of the working lumen 161 of the endovascular device is at least 29 French (Fr) (0.381 inches). According to some embodiments, the inner diameter (ID) of the working lumen 161 of the endovascular device is at least 30 French (Fr) (0.394 inches).

According to some such embodiments, the catheter is a catheter for intracranial applications. According to some such embodiments, the length of segment 110 of the endovascular device ranges from about 100.1% to about 120% of the length of the working segment 120 of the endovascular device. According to some such embodiments, the length of segment 110 of the endovascular device is at least 100.1% of the length of the working segment 120 of the endovascular device. According to some such embodiments, the length of segment 110 of the endovascular device is at least 100.2% of the length of the working segment 120 of the endovascular device. According to some such embodiments, the length of segment 110 of the endovascular device is at least 100.3% of the length of the working segment 120 of the endovascular device. According to some such embodiments, the length of segment 110 of the endovascular device is at least 100.4% of the length of the working segment 120 of the endovascular device. According to some such embodiments, the length of segment 110 of the endovascular device is at least 100.5% of the length of the working segment 120 of the endovascular device. According to some such embodiments, the length of segment 110 of the endovascular device is at least 100.6% of the length of the working segment 120 of the endovascular device. According to some such embodiments, the length of segment 110 of the endovascular device is at least 100.7% of the length of the working segment 120 of the endovascular device. According to some such embodiments, the length of segment 110 of the endovascular device is at least 100.8% of the length of the working segment 120 of the endovascular device. According to some such embodiments, the length of segment 110 of the endovascular device is at least 100.9% of the length of the working segment 120 of the endovascular device. According to some such embodiments, the length of segment 110 of the endovascular device is at least 101% of the length of the working segment 120 of the endovascular device. According to some such embodiments, the length of segment 110 of the endovascular device is at least 101.1% of the length of the working segment 120 of the endovascular device. According to some such embodiments, the length of segment 110 of the endovascular device is at least 101.2% of the length of the working segment 120 of the endovascular device. According to some such embodiments, the length of segment 110 of the endovascular device is at least 101.3% of the length of the working segment 120 of the endovascular device. According to some such embodiments, the length of segment 110 of the endovascular device is at least 101.4% of the length of the working segment 120 of the endovascular device. According to some such embodiments, the length of segment 110 of the endovascular device is at least 101.5% of the length of the working segment 120 of the endovascular device. According to some such embodiments, the length of segment 110 of the endovascular device is at least 101.6% of the length of the working segment 120 of the endovascular device. According to some such embodiments, the length of segment 110 of the endovascular device is at least 101.7% of the length of the working segment 120 of the endovascular device. According to some such embodiments, the length of segment 110 of the endovascular device is at least 101.8% of the length of the working segment 120 of the endovascular device. According to some such embodiments, the length of segment 110 of the endovascular device is at least 101.9% of the length of the working segment 120 of the endovascular device. According to some such embodiments, the length of segment 110 of the endovascular device is at least 102% of the length of the working segment 120 of the endovascular device. According to some such embodiments, the length of segment 110 of the endovascular device is at least 102.1% of the length of the working segment 120 of the endovascular device. According to some such embodiments, the length of segment 110 of the endovascular device is at least 102.2% of the length of the working segment 120 of the endovascular device. According to some such embodiments, the length of segment 110 of the endovascular device is at least 102.3% of the length of the working segment 120 of the endovascular device. According to some such embodiments, the length of segment 110 of the endovascular device is at least 102.4% of the length of the working segment 120 of the endovascular device. According to some such embodiments, the length of segment 110 of the endovascular device is at least 102.5% of the length of the working segment 120 of the endovascular device. According to some such embodiments, the length of segment 110 of the endovascular device is at least 102.6% of the length of the working segment 120 of the endovascular device. According to some such embodiments, the length of segment 110 of the endovascular device is at least 102.7% of the length of the working segment 120 of the endovascular device. According to some such embodiments, the length of segment 110 of the endovascular device is at least 102.8% of the length of the working segment 120 of the endovascular device. According to some such embodiments, the length of segment 110 of the endovascular device is at least 102.9% of the length of the working segment 120 of the endovascular device. According to some such embodiments, the length of segment 110 of the endovascular device is at least 103% of the length of the working segment 120 of the endovascular device. According to some such embodiments, the length of segment 110 of the endovascular device is at least 103.1% of the length of the working segment 120 of the endovascular device. According to some such embodiments, the length of segment 110 of the endovascular device is at least 103.2% of the length of the working segment 120 of the endovascular device. According to some such embodiments, the length of segment 110 of the endovascular device is at least 103.3% of the length of the working segment 120 of the endovascular device. According to some such embodiments, the length of segment 110 of the endovascular device is at least 103.4% of the length of the working segment 120 of the endovascular device. According to some such embodiments, the length of segment 110 of the endovascular device is at least 103.5% of the length of the working segment 120 of the endovascular device. According to some such embodiments, the length of segment 110 of the endovascular device is at least 103.6% of the length of the working segment 120 of the endovascular device. According to some such embodiments, the length of segment 110 of the endovascular device is at least 103.7% of the length of the working segment 120 of the endovascular device. According to some such embodiments, the length of segment 110 of the endovascular device is at least 103.8% of the length of the working segment 120 of the endovascular device. According to some such embodiments, the length of segment 110 of the endovascular device is at least 103.9% of the length of the working segment 120 of the endovascular device. According to some such embodiments, the length of segment 110 of the endovascular device is at least 104% of the length of the working segment 120 of the endovascular device. According to some such embodiments, the length of segment 110 of the endovascular device is at least 104.1% of the length of the working segment 120 of the endovascular device. According to some such embodiments, the length of segment 110 of the endovascular device is at least 104.2% of the length of the working segment 120 of the endovascular device. According to some such embodiments, the length of segment 110 of the endovascular device is at least 104.3% of the length of the working segment 120 of the endovascular device. According to some such embodiments, the length of segment 110 of the endovascular device is at least 104.4% of the length of the working segment 120 of the endovascular device. According to some such embodiments, the length of segment 110 of the endovascular device is at least 104.5% of the length of the working segment 120 of the endovascular device. According to some such embodiments, the length of segment 110 of the endovascular device is at least 104.6% of the length of the working segment 120 of the endovascular device. According to some such embodiments, the length of segment 110 of the endovascular device is at least 104.7% of the length of the working segment 120 of the endovascular device. According to some such embodiments, the length of segment 110 of the endovascular device is at least 104.8% of the length of the working segment 120 of the endovascular device. According to some such embodiments, the length of segment 110 of the endovascular device is at least 104.9% of the length of the working segment 120 of the endovascular device. According to some such embodiments, the length of segment 110 of the endovascular device is at least 105% of the length of the working segment 120 of the endovascular device. According to some such embodiments, the length of segment 110 of the endovascular device is at least 105.1% of the length of the working segment 120 of the endovascular device. According to some such embodiments, the length of segment 110 of the endovascular device is at least 105.2% of the length of the working segment 120 of the endovascular device. According to some such embodiments, the length of segment 110 of the endovascular device is at least 105.3% of the length of the working segment 120 of the endovascular device. According to some such embodiments, the length of segment 110 of the endovascular device is at least 105.4% of the length of the working segment 120 of the endovascular device. According to some such embodiments, the length of segment 110 of the endovascular device is at least 105.5% of the length of the working segment 120 of the endovascular device. According to some such embodiments, the length of segment 110 of the endovascular device is at least 105.6% of the length of the working segment 120 of the endovascular device. According to some such embodiments, the length of segment 110 of the endovascular device is at least 105.7% of the length of the working segment 120 of the endovascular device. According to some such embodiments, the length of segment 110 of the endovascular device is at least 105.8% of the length of the working segment 120 of the endovascular device. According to some such embodiments, the length of segment 110 of the endovascular device is at least 105.9% of the length of the working segment 120 of the endovascular device. According to some such embodiments, the length of segment 110 of the endovascular device is at least 106% of the length of the working segment 120 of the endovascular device. According to some such embodiments, the length of segment 110 of the endovascular device is at least 106.1% of the length of the working segment 120 of the endovascular device. According to some such embodiments, the length of segment 110 of the endovascular device is at least 106.2% of the length of the working segment 120 of the endovascular device. According to some such embodiments, the length of segment 110 of the endovascular device is at least 106.3% of the length of the working segment 120 of the endovascular device. According to some such embodiments, the length of segment 110 of the endovascular device is at least 106.4% of the length of the working segment 120 of the endovascular device. According to some such embodiments, the length of segment 110 of the endovascular device is at least 106.5% of the length of the working segment 120 of the endovascular device. According to some such embodiments, the length of segment 110 of the endovascular device is at least 106.6% of the length of the working segment 120 of the endovascular device. According to some such embodiments, the length of segment 110 of the endovascular device is at least 106.7% of the length of the working segment 120 of the endovascular device. According to some such embodiments, the length of segment 110 of the endovascular device is at least 106.8% of the length of the working segment 120 of the endovascular device. According to some such embodiments, the length of segment 110 of the endovascular device is at least 106.9% of the length of the working segment 120 of the endovascular device. According to some such embodiments, the length of segment 110 of the endovascular device is at least 107% of the length of the working segment 120 of the endovascular device. According to some such embodiments, the length of segment 110 of the endovascular device is at least 107.1% of the length of the working segment 120 of the endovascular device. According to some such embodiments, the length of segment 110 of the endovascular device is at least 107.2% of the length of the working segment 120 of the endovascular device. According to some such embodiments, the length of segment 110 of the endovascular device is at least 107.3% of the length of the working segment 120 of the endovascular device. According to some such embodiments, the length of segment 110 of the endovascular device is at least 107.4% of the length of the working segment 120 of the endovascular device. According to some such embodiments, the length of segment 110 of the endovascular device is at least 107.5% of the length of the working segment 120 of the endovascular device. According to some such embodiments, the length of segment 110 of the endovascular device is at least 107.6% of the length of the working segment 120 of the endovascular device. According to some such embodiments, the length of segment 110 of the endovascular device is at least 107.7% of the length of the working segment 120 of the endovascular device. According to some such embodiments, the length of segment 110 of the endovascular device is at least 107.8% of the length of the working segment 120 of the endovascular device. According to some such embodiments, the length of segment 110 of the endovascular device is at least 107.9% of the length of the working segment 120 of the endovascular device. According to some such embodiments, the length of segment 110 of the endovascular device is at least 108% of the length of the working segment 120 of the endovascular device. According to some such embodiments, the length of segment 110 of the endovascular device is at least 108.1% of the length of the working segment 120 of the endovascular device. According to some such embodiments, the length of segment 110 of the endovascular device is at least 108.2% of the length of the working segment 120 of the endovascular device. According to some such embodiments, the length of segment 110 of the endovascular device is at least 108.3% of the length of the working segment 120 of the endovascular device. According to some such embodiments, the length of segment 110 of the endovascular device is at least 108.4% of the length of the working segment 120 of the endovascular device. According to some such embodiments, the length of segment 110 of the endovascular device is at least 108.5% of the length of the working segment 120 of the endovascular device. According to some such embodiments, the length of segment 110 of the endovascular device is at least 108.6% of the length of the working segment 120 of the endovascular device. According to some such embodiments, the length of segment 110 of the endovascular device is at least 108.7% of the length of the working segment 120 of the endovascular device. According to some such embodiments, the length of segment 110 of the endovascular device is at least 108.8% of the length of the working segment 120 of the endovascular device. According to some such embodiments, the length of segment 110 of the endovascular device is at least 108.9% of the length of the working segment 120 of the endovascular device. According to some such embodiments, the length of segment 110 of the endovascular device is at least 109% of the length of the working segment 120 of the endovascular device. According to some such embodiments, the length of segment 110 of the endovascular device is at least 109.1% of the length of the working segment 120 of the endovascular device. According to some such embodiments, the length of segment 110 of the endovascular device is at least 109.2% of the length of the working segment 120 of the endovascular device. According to some such embodiments, the length of segment 110 of the endovascular device is at least 109.3% of the length of the working segment 120 of the endovascular device. According to some such embodiments, the length of segment 110 of the endovascular device is at least 109.4% of the length of the working segment 120 of the endovascular device. According to some such embodiments, the length of segment 110 of the endovascular device is at least 109.5% of the length of the working segment 120 of the endovascular device. According to some embodiments, the length of segment 110 of the endovascular device is at least 109.6% of the length of the working segment 120 of the endovascular device. According to some embodiments, the length of segment 110 of the endovascular device is at least 109.7% of the length of the working segment 120 of the endovascular device. According to some embodiments, the length of segment 110 of the endovascular device is at least 109.8% of the length of the working segment 120 of the endovascular device. According to some embodiments, the length of segment 110 of the endovascular device is at least 109.9% of the length of the working segment 120 of the endovascular device. According to some such embodiments, the length of segment 110 of the endovascular device is at least 110% of the length of the working segment 120 of the endovascular device. According to some such embodiments, the length of segment 110 of the endovascular device is at least 110.1% of the length of the working segment 120 of the endovascular device. According to some such embodiments, the length of segment 110 of the endovascular device is at least 110.2% of the length of the working segment 120 of the endovascular device. According to some such embodiments, the length of segment 110 of the endovascular device is at least 110.3% of the length of the working segment 120 of the endovascular device. According to some such embodiments, the length of segment 110 of the endovascular device is at least 110.4% of the length of the working segment 120 of the endovascular device. According to some such embodiments, the length of segment 110 of the endovascular device is at least 110.5% of the length of the working segment 120 of the endovascular device. According to some such embodiments, the length of segment 110 of the endovascular device is at least 110.6% of the length of the working segment 120 of the endovascular device. According to some such embodiments, the length of segment 110 of the endovascular device is at least 110.7% of the length of the working segment 120 of the endovascular device. According to some such embodiments, the length of segment 110 of the endovascular device is at least 110.8% of the length of the working segment 120 of the endovascular device. According to some such embodiments, the length of segment 110 of the endovascular device is at least 110.9% of the length of the working segment 120 of the endovascular device. According to some such embodiments, the length of segment 110 of the endovascular device is at least 111% of the length of the working segment 120 of the endovascular device. According to some such embodiments, the length of segment 110 of the endovascular device is at least 111.1% of the length of the working segment 120 of the endovascular device. According to some such embodiments, the length of segment 110 of the endovascular device is at least 111.2% of the length of the working segment 120 of the endovascular device. According to some such embodiments, the length of segment 110 of the endovascular device is at least 111.3% of the length of the working segment 120 of the endovascular device. According to some such embodiments, the length of segment 110 of the endovascular device is at least 111.4% of the length of the working segment 120 of the endovascular device. According to some such embodiments, the length of segment 110 of the endovascular device is at least 111.5% of the length of the working segment 120 of the endovascular device. According to some such embodiments, the length of segment 110 of the endovascular device is at least 111.6% of the length of the working segment 120 of the endovascular device. According to some such embodiments, the length of segment 110 of the endovascular device is at least 111.7% of the length of the working segment 120 of the endovascular device. According to some such embodiments, the length of segment 110 of the endovascular device is at least 111.8% of the length of the working segment 120 of the endovascular device. According to some such embodiments, the length of segment 110 of the endovascular device is at least 111.9% of the length of the working segment 120 of the endovascular device. According to some such embodiments, the length of segment 110 of the endovascular device is at least 112% of the length of the working segment 120 of the endovascular device. According to some such embodiments, the length of segment 110 of the endovascular device is at least 112.1% of the length of the working segment 120 of the endovascular device. According to some such embodiments, the length of segment 110 of the endovascular device is at least 112.2% of the length of the working segment 120 of the endovascular device. According to some such embodiments, the length of segment 110 of the endovascular device is at least 112.3% of the length of the working segment 120 of the endovascular device. According to some such embodiments, the length of segment 110 of the endovascular device is at least 112.4% of the length of the working segment 120 of the endovascular device. According to some such embodiments, the length of segment 110 of the endovascular device is at least 112.5% of the length of the working segment 120 of the endovascular device. According to some such embodiments, the length of segment 110 of the endovascular device is at least 112.6% of the length of the working segment 120 of the endovascular device. According to some such embodiments, the length of segment 110 of the endovascular device is at least 112.7% of the length of the working segment 120 of the endovascular device. According to some such embodiments, the length of segment 110 of the endovascular device is at least 112.8% of the length of the working segment 120 of the endovascular device. According to some such embodiments, the length of segment 110 of the endovascular device is at least 112.9% of the length of the working segment 120 of the endovascular device. According to some such embodiments, the length of segment 110 of the endovascular device is at least 113% of the length of the working segment 120 of the endovascular device. According to some such embodiments, the length of segment 110 of the endovascular device is at least 113.1% of the length of the working segment 120 of the endovascular device. According to some such embodiments, the length of segment 110 of the endovascular device is at least 113.2% of the length of the working segment 120 of the endovascular device. According to some such embodiments, the length of segment 110 of the endovascular device is at least 113.3% of the length of the working segment 120 of the endovascular device. According to some such embodiments, the length of segment 110 of the endovascular device is at least 113.4% of the length of the working segment 120 of the endovascular device. According to some such embodiments, the length of segment 110 of the endovascular device is at least 113.5% of the length of the working segment 120 of the endovascular device. According to some such embodiments, the length of segment 110 of the endovascular device is at least 113.6% of the length of the working segment 120 of the endovascular device. According to some such embodiments, the length of segment 110 of the endovascular device is at least 113.7% of the length of the working segment 120 of the endovascular device. According to some such embodiments, the length of segment 110 of the endovascular device is at least 113.8% of the length of the working segment 120 of the endovascular device. According to some such embodiments, the length of segment 110 of the endovascular device is at least 113.9% of the length of the working segment 120 of the endovascular device. According to some such embodiments, the length of segment 110 of the endovascular device is at least 114% of the length of the working segment 120 of the endovascular device. According to some such embodiments, the length of segment 110 of the endovascular device is at least 114.1% of the length of the working segment 120 of the endovascular device. According to some such embodiments, the length of segment 110 of the endovascular device is at least 114.2% of the length of the working segment 120 of the endovascular device. According to some such embodiments, the length of segment 110 of the endovascular device is at least 114.3% of the length of the working segment 120 of the endovascular device. According to some such embodiments, the length of segment 110 of the endovascular device is at least 114.4% of the length of the working segment 120 of the endovascular device. According to some such embodiments, the length of segment 110 of the endovascular device is at least 114.5% of the length of the working segment 120 of the endovascular device. According to some such embodiments, the length of segment 110 of the endovascular device is at least 114.6% of the length of the working segment 120 of the endovascular device. According to some such embodiments, the length of segment 110 of the endovascular device is at least 114.7% of the length of the working segment 120 of the endovascular device. According to some such embodiments, the length of segment 110 of the endovascular device is at least 114.8% of the length of the working segment 120 of the endovascular device. According to some such embodiments, the length of segment 110 of the endovascular device is at least 114.9% of the length of the working segment 120 of the endovascular device. According to some such embodiments, the length of segment 110 of the endovascular device is at least 115% of the length of the working segment 120 of the endovascular device. According to some such embodiments, the length of segment 110 of the endovascular device is at least 115.1% of the length of the working segment 120 of the endovascular device. According to some such embodiments, the length of segment 110 of the endovascular device is at least 115.2% of the length of the working segment 120 of the endovascular device. According to some such embodiments, the length of segment 110 of the endovascular device is at least 115.3% of the length of the working segment 120 of the endovascular device. According to some such embodiments, the length of segment 110 of the endovascular device is at least 115.4% of the length of the working segment 120 of the endovascular device. According to some such embodiments, the length of segment 110 of the endovascular device is at least 115.5% of the length of the working segment 120 of the endovascular device. According to some such embodiments, the length of segment 110 of the endovascular device is at least 115.6% of the length of the working segment 120 of the endovascular device. According to some such embodiments, the length of segment 110 of the endovascular device is at least 115.7% of the length of the working segment 120 of the endovascular device. According to some such embodiments, the length of segment 110 of the endovascular device is at least 115.8% of the length of the working segment 120 of the endovascular device. According to some such embodiments, the length of segment 110 of the endovascular device is at least 115.9% of the length of the working segment 120 of the endovascular device. According to some such embodiments, the length of segment 110 of the endovascular device is at least 116% of the length of the working segment 120 of the endovascular device. According to some such embodiments, the length of segment 110 of the endovascular device is at least 116.1% of the length of the working segment 120 of the endovascular device. According to some such embodiments, the length of segment 110 of the endovascular device is at least 116.2% of the length of the working segment 120 of the endovascular device. According to some such embodiments, the length of segment 110 of the endovascular device is at least 116.3% of the length of the working segment 120 of the endovascular device. According to some such embodiments, the length of segment 110 of the endovascular device is at least 116.4% of the length of the working segment 120 of the endovascular device. According to some such embodiments, the length of segment 110 of the endovascular device is at least 116.5% of the length of the working segment 120 of the endovascular device. According to some such embodiments, the length of segment 110 of the endovascular device is at least 116.6% of the length of the working segment 120 of the endovascular device. According to some such embodiments, the length of segment 110 of the endovascular device is at least 116.7% of the length of the working segment 120 of the endovascular device. According to some such embodiments, the length of segment 110 of the endovascular device is at least 116.8% of the length of the working segment 120 of the endovascular device. According to some such embodiments, the length of segment 110 of the endovascular device is at least 116.9% of the length of the working segment 120 of the endovascular device. According to some such embodiments, the length of segment 110 of the endovascular device is at least 117% of the length of the working segment 120 of the endovascular device. According to some such embodiments, the length of segment 110 of the endovascular device is at least 117.1% of the length of the working segment 120 of the endovascular device. According to some such embodiments, the length of segment 110 of the endovascular device is at least 117.2% of the length of the working segment 120 of the endovascular device. According to some such embodiments, the length of segment 110 of the endovascular device is at least 117.3% of the length of the working segment 120 of the endovascular device. According to some such embodiments, the length of segment 110 of the endovascular device is at least 117.4% of the length of the working segment 120 of the endovascular device. According to some such embodiments, the length of segment 110 of the endovascular device is at least 117.5% of the length of the working segment 120 of the endovascular device. According to some such embodiments, the length of segment 110 of the endovascular device is at least 117.6% of the length of the working segment 120 of the endovascular device. According to some such embodiments, the length of segment 110 of the endovascular device is at least 117.7% of the length of the working segment 120 of the endovascular device. According to some such embodiments, the length of segment 110 of the endovascular device is at least 117.8% of the length of the working segment 120 of the endovascular device. According to some such embodiments, the length of segment 110 of the endovascular device is at least 117.9% of the length of the working segment 120 of the endovascular device. According to some such embodiments, the length of segment 110 of the endovascular device is at least 118% of the length of the working segment 120 of the endovascular device. According to some such embodiments, the length of segment 110 of the endovascular device is at least 118.1% of the length of the working segment 120 of the endovascular device. According to some such embodiments, the length of segment 110 of the endovascular device is at least 118.2% of the length of the working segment 120 of the endovascular device. According to some such embodiments, the length of segment 110 of the endovascular device is at least 118.3% of the length of the working segment 120 of the endovascular device. According to some such embodiments, the length of segment 110 of the endovascular device is at least 118.4% of the length of the working segment 120 of the endovascular device. According to some such embodiments, the length of segment 110 of the endovascular device is at least 118.5% of the length of the working segment 120 of the endovascular device. According to some such embodiments, the length of segment 110 of the endovascular device is at least 118.6% of the length of the working segment 120 of the endovascular device. According to some such embodiments, the length of segment 110 of the endovascular device is at least 118.7% of the length of the working segment 120 of the endovascular device. According to some such embodiments, the length of segment 110 of the endovascular device is at least 118.8% of the length of the working segment 120 of the endovascular device. According to some such embodiments, the length of segment 110 of the endovascular device is at least 118.9% of the length of the working segment 120 of the endovascular device. According to some such embodiments, the length of segment 110 of the endovascular device is at least 119% of the length of the working segment 120 of the endovascular device. According to some such embodiments, the length of segment 110 of the endovascular device is at least 119.1% of the length of the working segment 120 of the endovascular device. According to some such embodiments, the length of segment 110 of the endovascular device is at least 119.2% of the length of the working segment 120 of the endovascular device. According to some such embodiments, the length of segment 110 of the endovascular device is at least 119.3% of the length of the working segment 120 of the endovascular device. According to some such embodiments, the length of segment 110 of the endovascular device is at least 119.4% of the length of the working segment 120 of the endovascular device. According to some such embodiments, the length of segment 110 of the endovascular device is at least 119.5% of the length of the working segment 120 of the endovascular device. According to some such embodiments, the length of segment 110 of the endovascular device is at least 119.6% of the length of the working segment 120 of the endovascular device. According to some such embodiments, the length of segment 110 of the endovascular device is at least 119.7% of the length of the working segment 120 of the endovascular device. According to some such embodiments, the length of segment 110 of the endovascular device is at least 119.8% of the length of the working segment 120 of the endovascular device. According to some such embodiments, the length of segment 110 of the endovascular device is at least 119.9% of the length of the working segment 120 of the endovascular device. According to some such embodiments, the length of segment 110 of the endovascular device is about 120% of the length of the working segment 120 of the endovascular device.

According to some such embodiments, the catheter is useful for peripheral applications. According to some such embodiments, the length of segment 110 of the endovascular device ranges from at least 105% to about 300% of the length of the working segment 120 of the endovascular device. According to some such embodiments, the length of segment 110 of the endovascular device is at least 105.1% of the length of the working segment 120 of the endovascular device. According to some such embodiments, the length of segment 110 of the endovascular device is at least 105.2% of the length of the working segment 120 of the endovascular device. According to some such embodiments, the length of segment 110 of the endovascular device is at least 105.3% of the length of the working segment 120 of the endovascular device. According to some such embodiments, the length of segment 110 of the endovascular device is at least 105.4% of the length of the working segment 120 of the endovascular device. According to some such embodiments, the length of segment 110 of the endovascular device is at least 105.5% of the length of the working segment 120 of the endovascular device. According to some such embodiments, the length of segment 110 of the endovascular device is at least 105.6% of the length of the working segment 120 of the endovascular device. According to some such embodiments, the length of segment 110 of the endovascular device is at least 105.7% of the length of the working segment 120 of the endovascular device. According to some such embodiments, the length of segment 110 of the endovascular device is at least 105.8% of the length of the working segment 120 of the endovascular device. According to some such embodiments, the length of segment 110 of the endovascular device is at least 105.9% of the length of the working segment 120 of the endovascular device. According to some such embodiments, the length of segment 110 of the endovascular device is at least 106% of the length of the working segment 120 of the endovascular device. According to some such embodiments, the length of segment 110 of the endovascular device is at least 106.1% of the length of the working segment 120 of the endovascular device. According to some such embodiments, the length of segment 110 of the endovascular device is at least 106.2% of the length of the working segment 120 of the endovascular device. According to some such embodiments, the length of segment 110 of the endovascular device is at least 106.3% of the length of the working segment 120 of the endovascular device. According to some such embodiments, the length of segment 110 of the endovascular device is at least 106.4% of the length of the working segment 120 of the endovascular device. According to some such embodiments, the length of segment 110 of the endovascular device is at least 106.5% of the length of the working segment 120 of the endovascular device. According to some such embodiments, the length of segment 110 of the endovascular device is at least 106.6% of the length of the working segment 120 of the endovascular device. According to some such embodiments, the length of segment 110 of the endovascular device is at least 106.7% of the length of the working segment 120 of the endovascular device. According to some such embodiments, the length of segment 110 of the endovascular device is at least 106.8% of the length of the working segment 120 of the endovascular device. According to some such embodiments, the length of segment 110 of the endovascular device is at least 106.9% of the length of the working segment 120 of the endovascular device. According to some such embodiments, the length of segment 110 of the endovascular device is at least 107% of the length of the working segment 120 of the endovascular device. According to some such embodiments, the length of segment 110 of the endovascular device is at least 107.1% of the length of the working segment 120 of the endovascular device. According to some such embodiments, the length of segment 110 of the endovascular device is at least 107.2% of the length of the working segment 120 of the endovascular device. According to some such embodiments, the length of segment 110 of the endovascular device is at least 107.3% of the length of the working segment 120 of the endovascular device. According to some such embodiments, the length of segment 110 of the endovascular device is at least 107.4% of the length of the working segment 120 of the endovascular device. According to some such embodiments, the length of segment 110 of the endovascular device is at least 107.5% of the length of the working segment 120 of the endovascular device. According to some such embodiments, the length of segment 110 of the endovascular device is at least 107.6% of the length of the working segment 120 of the endovascular device. According to some such embodiments, the length of segment 110 of the endovascular device is at least 107.7% of the length of the working segment 120 of the endovascular device. According to some such embodiments, the length of segment 110 of the endovascular device is at least 107.8% of the length of the working segment 120 of the endovascular device. According to some such embodiments, the length of segment 110 of the endovascular device is at least 107.9% of the length of the working segment 120 of the endovascular device. According to some such embodiments, the length of segment 110 of the endovascular device is at least 108% of the length of the working segment 120 of the endovascular device. According to some such embodiments, the length of segment 110 of the endovascular device is at least 108.1% of the length of the working segment 120 of the endovascular device. According to some such embodiments, the length of segment 110 of the endovascular device is at least 108.2% of the length of the working segment 120 of the endovascular device. According to some such embodiments, the length of segment 110 of the endovascular device is at least 108.3% of the length of the working segment 120 of the endovascular device. According to some such embodiments, the length of segment 110 of the endovascular device is at least 108.4% of the length of the working segment 120 of the endovascular device. According to some such embodiments, the length of segment 110 of the endovascular device is at least 108.5% of the length of the working segment 120 of the endovascular device. According to some such embodiments, the length of segment 110 of the endovascular device is at least 108.6% of the length of the working segment 120 of the endovascular device. According to some such embodiments, the length of segment 110 of the endovascular device is at least 108.7% of the length of the working segment 120 of the endovascular device. According to some such embodiments, the length of segment 110 of the endovascular device is at least 108.8% of the length of the working segment 120 of the endovascular device. According to some such embodiments, the length of segment 110 of the endovascular device is at least 108.9% of the length of the working segment 120 of the endovascular device. According to some such embodiments, the length of segment 110 of the endovascular device is at least 109% of the length of the working segment 120 of the endovascular device. According to some such embodiments, the length of segment 110 of the endovascular device is at least 109.1% of the length of the working segment 120 of the endovascular device. According to some such embodiments, the length of segment 110 of the endovascular device is at least 109.2% of the length of the working segment 120 of the endovascular device. According to some such embodiments, the length of segment 110 of the endovascular device is at least 109.3% of the length of the working segment 120 of the endovascular device. According to some such embodiments, the length of segment 110 of the endovascular device is at least 109.4% of the length of the working segment 120 of the endovascular device. According to some such embodiments, the length of segment 110 of the endovascular device is at least 109.5% of the length of the working segment 120 of the endovascular device. According to some such embodiments, the length of segment 110 of the endovascular device is at least 109.6% of the length of the working segment 120 of the endovascular device. According to some such embodiments, the length of segment 110 of the endovascular device is at least 109.7% of the length of the working segment 120 of the endovascular device. According to some such embodiments, the length of segment 110 of the endovascular device is at least 109.8% of the length of the working segment 120 of the endovascular device. According to some such embodiments, the length of segment 110 of the endovascular device is at least 109.9% of the length of the working segment 120 of the endovascular device. According to some such embodiments, the length of segment 110 of the endovascular device is at least 110% of the length of the working segment 120 of the endovascular device. According to some such embodiments, the length of segment 110 of the endovascular device is at least 110.1% of the length of the working segment 120 of the endovascular device. According to some such embodiments, the length of segment 110 of the endovascular device is at least 110.2% of the length of the working segment 120 of the endovascular device. According to some such embodiments, the length of segment 110 of the endovascular device is at least 110.3% of the length of the working segment 120 of the endovascular device. According to some such embodiments, the length of segment 110 of the endovascular device is at least 110.4% of the length of the working segment 120 of the endovascular device. According to some such embodiments, the length of segment 110 of the endovascular device is at least 110.5% of the length of the working segment 120 of the endovascular device. According to some such embodiments, the length of segment 110 of the endovascular device is at least 110.6% of the length of the working segment 120 of the endovascular device. According to some such embodiments, the length of segment 110 of the endovascular device is at least 110.7% of the length of the working segment 120 of the endovascular device. According to some such embodiments, the length of segment 110 of the endovascular device is at least 110.8% of the length of the working segment 120 of the endovascular device. According to some such embodiments, the length of segment 110 of the endovascular device is at least 110.9% of the length of the working segment 120 of the endovascular device. According to some such embodiments, the length of segment 110 of the endovascular device is at least 111% of the length of the working segment 120 of the endovascular device. According to some such embodiments, the length of segment 110 of the endovascular device is at least 111.1% of the length of the working segment 120 of the endovascular device. According to some such embodiments, the length of segment 110 of the endovascular device is at least 111.2% of the length of the working segment 120 of the endovascular device. According to some such embodiments, the length of segment 110 of the endovascular device is at least 111.3% of the length of the working segment 120 of the endovascular device. According to some such embodiments, the length of segment 110 of the endovascular device is at least 111.4% of the length of the working segment 120 of the endovascular device. According to some such embodiments, the length of segment 110 of the endovascular device is at least 111.5% of the length of the working segment 120 of the endovascular device. According to some such embodiments, the length of segment 110 of the endovascular device is at least 111.6% of the length of the working segment 120 of the endovascular device. According to some such embodiments, the length of segment 110 of the endovascular device is at least 111.7% of the length of the working segment 120 of the endovascular device. According to some such embodiments, the length of segment 110 of the endovascular device is at least 111.8% of the length of the working segment 120 of the endovascular device. According to some such embodiments, the length of segment 110 of the endovascular device is at least 111.9% of the length of the working segment 120 of the endovascular device. According to some such embodiments, the length of segment 110 of the endovascular device is at least 112% of the length of the working segment 120 of the endovascular device. According to some such embodiments, the length of segment 110 of the endovascular device is at least 112.1% of the length of the working segment 120 of the endovascular device. According to some such embodiments, the length of segment 110 of the endovascular device is at least 112.2% of the length of the working segment 120 of the endovascular device. According to some such embodiments, the length of segment 110 of the endovascular device is at least 112.3% of the length of the working segment 120 of the endovascular device. According to some such embodiments, the length of segment 110 of the endovascular device is at least 112.4% of the length of the working segment 120 of the endovascular device. According to some such embodiments, the length of segment 110 of the endovascular device is at least 112.5% of the length of the working segment 120 of the endovascular device. According to some such embodiments, the length of segment 110 of the endovascular device is at least 112.6% of the length of the working segment 120 of the endovascular device. According to some such embodiments, the length of segment 110 of the endovascular device is at least 112.7% of the length of the working segment 120 of the endovascular device. According to some such embodiments, the length of segment 110 of the endovascular device is at least 112.8% of the length of the working segment 120 of the endovascular device. According to some such embodiments, the length of segment 110 of the endovascular device is at least 112.9% of the length of the working segment 120 of the endovascular device. According to some such embodiments, the length of segment 110 of the endovascular device is at least 113% of the length of the working segment 120 of the endovascular device. According to some such embodiments, the length of segment 110 of the endovascular device is at least 113.1% of the length of the working segment 120 of the endovascular device. According to some such embodiments, the length of segment 110 of the endovascular device is at least 113.2% of the length of the working segment 120 of the endovascular device. According to some such embodiments, the length of segment 110 of the endovascular device is at least 113.3% of the length of the working segment 120 of the endovascular device. According to some such embodiments, the length of segment 110 of the endovascular device is at least 113.4% of the length of the working segment 120 of the endovascular device. According to some such embodiments, the length of segment 110 of the endovascular device is at least 113.5% of the length of the working segment 120 of the endovascular device. According to some such embodiments, the length of segment 110 of the endovascular device is at least 113.6% of the length of the working segment 120 of the endovascular device. According to some such embodiments, the length of segment 110 of the endovascular device is at least 113.7% of the length of the working segment 120 of the endovascular device. According to some such embodiments, the length of segment 110 of the endovascular device is at least 113.8% of the length of the working segment 120 of the endovascular device. According to some such embodiments, the length of segment 110 of the endovascular device is at least 113.9% of the length of the working segment 120 of the endovascular device. According to some such embodiments, the length of segment 110 of the endovascular device is at least 114% of the length of the working segment 120 of the endovascular device. According to some such embodiments, the length of segment 110 of the endovascular device is at least 114.1% of the length of the working segment 120 of the endovascular device. According to some such embodiments, the length of segment 110 of the endovascular device is at least 114.2% of the length of the working segment 120 of the endovascular device. According to some such embodiments, the length of segment 110 of the endovascular device is at least 114.3% of the length of the working segment 120 of the endovascular device. According to some such embodiments, the length of segment 110 of the endovascular device is at least 114.4% of the length of the working segment 120 of the endovascular device. According to some such embodiments, the length of segment 110 of the endovascular device is at least 114.5% of the length of the working segment 120 of the endovascular device. According to some such embodiments, the length of segment 110 of the endovascular device is at least 114.6% of the length of the working segment 120 of the endovascular device. According to some such embodiments, the length of segment 110 of the endovascular device is at least 114.7% of the length of the working segment 120 of the endovascular device. According to some such embodiments, the length of segment 110 of the endovascular device is at least 114.8% of the length of the working segment 120 of the endovascular device. According to some such embodiments, the length of segment 110 of the endovascular device is at least 114.9% of the length of the working segment 120 of the endovascular device. According to some such embodiments, the length of segment 110 of the endovascular device is at least 115% of the length of the working segment 120 of the endovascular device. According to some such embodiments, the length of segment 110 of the endovascular device is at least 115.1% of the length of the working segment 120 of the endovascular device. According to some such embodiments, the length of segment 110 of the endovascular device is at least 115.2% of the length of the working segment 120 of the endovascular device. According to some such embodiments, the length of segment 110 of the endovascular device is at least 115.3% of the length of the working segment 120 of the endovascular device. According to some such embodiments, the length of segment 110 of the endovascular device is at least 115.4% of the length of the working segment 120 of the endovascular device. According to some such embodiments, the length of segment 110 of the endovascular device is at least 115.5% of the length of the working segment 120 of the endovascular device. According to some such embodiments, the length of segment 110 of the endovascular device is at least 115.6% of the length of the working segment 120 of the endovascular device. According to some such embodiments, the length of segment 110 of the endovascular device is at least 115.7% of the length of the working segment 120 of the endovascular device. According to some such embodiments, the length of segment 110 of the endovascular device is at least 115.8% of the length of the working segment 120 of the endovascular device. According to some such embodiments, the length of segment 110 of the endovascular device is at least 115.9% of the length of the working segment 120 of the endovascular device. According to some such embodiments, the length of segment 110 of the endovascular device is at least 116% of the length of the working segment 120 of the endovascular device. According to some such embodiments, the length of segment 110 of the endovascular device is at least 116.1% of the length of the working segment 120 of the endovascular device. According to some such embodiments, the length of segment 110 of the endovascular device is at least 116.2% of the length of the working segment 120 of the endovascular device. According to some such embodiments, the length of segment 110 of the endovascular device is at least 116.3% of the length of the working segment 120 of the endovascular device. According to some such embodiments, the length of segment 110 of the endovascular device is at least 116.4% of the length of the working segment 120 of the endovascular device. According to some such embodiments, the length of segment 110 of the endovascular device is at least 116.5% of the length of the working segment 120 of the endovascular device. According to some such embodiments, the length of segment 110 of the endovascular device is at least 116.6% of the length of the working segment 120 of the endovascular device. According to some such embodiments, the length of segment 110 of the endovascular device is at least 116.7% of the length of the working segment 120 of the endovascular device. According to some such embodiments, the length of segment 110 of the endovascular device is at least 116.8% of the length of the working segment 120 of the endovascular device. According to some such embodiments, the length of segment 110 of the endovascular device is at least 116.9% of the length of the working segment 120 of the endovascular device. According to some such embodiments, the length of segment 110 of the endovascular device is at least 117% of the length of the working segment 120 of the endovascular device. According to some such embodiments, the length of segment 110 of the endovascular device is at least 117.1% of the length of the working segment 120 of the endovascular device. According to some such embodiments, the length of segment 110 of the endovascular device is at least 117.2% of the length of the working segment 120 of the endovascular device. According to some such embodiments, the length of segment 110 of the endovascular device is at least 117.3% of the length of the working segment 120 of the endovascular device. According to some such embodiments, the length of segment 110 of the endovascular device is at least 117.4% of the length of the working segment 120 of the endovascular device. According to some such embodiments, the length of segment 110 of the endovascular device is at least 117.5% of the length of the working segment 120 of the endovascular device. According to some such embodiments, the length of segment 110 of the endovascular device is at least 117.6% of the length of the working segment 120 of the endovascular device. According to some such embodiments, the length of segment 110 of the endovascular device is at least 117.7% of the length of the working segment 120 of the endovascular device. According to some such embodiments, the length of segment 110 of the endovascular device is at least 117.8% of the length of the working segment 120 of the endovascular device. According to some such embodiments, the length of segment 110 of the endovascular device is at least 117.9% of the length of the working segment 120 of the endovascular device. According to some such embodiments, the length of segment 110 of the endovascular device is at least 118% of the length of the working segment 120 of the endovascular device. According to some such embodiments, the length of segment 110 of the endovascular device is at least 118.1% of the length of the working segment 120 of the endovascular device. According to some such embodiments, the length of segment 110 of the endovascular device is at least 118.2% of the length of the working segment 120 of the endovascular device. According to some such embodiments, the length of segment 110 of the endovascular device is at least 118.3% of the length of the working segment 120 of the endovascular device. According to some such embodiments, the length of segment 110 of the endovascular device is at least 118.4% of the length of the working segment 120 of the endovascular device. According to some such embodiments, the length of segment 110 of the endovascular device is at least 118.5% of the length of the working segment 120 of the endovascular device. According to some such embodiments, the length of segment 110 of the endovascular device is at least 118.6% of the length of the working segment 120 of the endovascular device. According to some such embodiments, the length of segment 110 of the endovascular device is at least 118.7% of the length of the working segment 120 of the endovascular device. According to some such embodiments, the length of segment 110 of the endovascular device is at least 118.8% of the length of the working segment 120 of the endovascular device. According to some such embodiments, the length of segment 110 of the endovascular device is at least 118.9% of the length of the working segment 120 of the endovascular device. According to some such embodiments, the length of segment 110 of the endovascular device is at least 119% of the length of the working segment 120 of the endovascular device. According to some such embodiments, the length of segment 110 of the endovascular device is at least 119.1% of the length of the working segment 120 of the endovascular device. According to some such embodiments, the length of segment 110 of the endovascular device is at least 119.2% of the length of the working segment 120 of the endovascular device. According to some such embodiments, the length of segment 110 of the endovascular device is at least 119.3% of the length of the working segment 120 of the endovascular device. According to some such embodiments, the length of segment 110 of the endovascular device is at least 119.4% of the length of the working segment 120 of the endovascular device. According to some such embodiments, the length of segment 110 of the endovascular device is at least 119.5% of the length of the working segment 120 of the endovascular device. According to some such embodiments, the length of segment 110 of the endovascular device is at least 119.6% of the length of the working segment 120 of the endovascular device. According to some such embodiments, the length of segment 110 of the endovascular device is at least 119.7% of the length of the working segment 120 of the endovascular device. According to some such embodiments, the length of segment 110 of the endovascular device is at least 119.8% of the length of the working segment 120 of the endovascular device. According to some such embodiments, the length of segment 110 of the endovascular device is at least 119.9% of the length of the working segment 120 of the endovascular device. According to some such embodiments, the length of segment 110 of the endovascular device is at least 120% of the length of the working segment 120 of the endovascular device. According to some such embodiments, the length of segment 110 of the endovascular device is at least 120.1% of the length of the working segment 120 of the endovascular device. According to some such embodiments, the length of segment 110 of the endovascular device is at least 120.2% of the length of the working segment 120 of the endovascular device. According to some such embodiments, the length of segment 110 of the endovascular device is at least 120.3% of the length of the working segment 120 of the endovascular device. According to some such embodiments, the length of segment 110 of the endovascular device is at least 120.4% of the length of the working segment 120 of the endovascular device. According to some such embodiments, the length of segment 110 of the endovascular device is at least 120.5% of the length of the working segment 120 of the endovascular device. According to some such embodiments, the length of segment 110 of the endovascular device is at least 120.6% of the length of the working segment 120 of the endovascular device. According to some such embodiments, the length of segment 110 of the endovascular device is at least 120.7% of the length of the working segment 120 of the endovascular device. According to some such embodiments, the length of segment 110 of the endovascular device is at least 120.8% of the length of the working segment 120 of the endovascular device. According to some such embodiments, the length of segment 110 of the endovascular device is at least 120.9% of the length of the working segment 120 of the endovascular device. According to some such embodiments, the length of segment 110 of the endovascular device is at least 121% of the length of the working segment 120 of the endovascular device. According to some such embodiments, the length of segment 110 of the endovascular device is at least 121.1% of the length of the working segment 120 of the endovascular device. According to some such embodiments, the length of segment 110 of the endovascular device is at least 121.2% of the length of the working segment 120 of the endovascular device. According to some such embodiments, the length of segment 110 of the endovascular device is at least 121.3% of the length of the working segment 120 of the endovascular device. According to some such embodiments, the length of segment 110 of the endovascular device is at least 121.4% of the length of the working segment 120 of the endovascular device. According to some such embodiments, the length of segment 110 of the endovascular device is at least 121.5% of the length of the working segment 120 of the endovascular device. According to some such embodiments, the length of segment 110 of the endovascular device is at least 121.6% of the length of the working segment 120 of the endovascular device. According to some such embodiments, the length of segment 110 of the endovascular device is at least 121.7% of the length of the working segment 120 of the endovascular device. According to some such embodiments, the length of segment 110 of the endovascular device is at least 121.8% of the length of the working segment 120 of the endovascular device. According to some such embodiments, the length of segment 110 of the endovascular device is at least 121.9% of the length of the working segment 120 of the endovascular device. According to some such embodiments, the length of segment 110 of the endovascular device is at least 122% of the length of the working segment 120 of the endovascular device. According to some such embodiments, the length of segment 110 of the endovascular device is at least 122.1% of the length of the working segment 120 of the endovascular device. According to some such embodiments, the length of segment 110 of the endovascular device is at least 122.2% of the length of the working segment 120 of the endovascular device. According to some such embodiments, the length of segment 110 of the endovascular device is at least 122.3% of the length of the working segment 120 of the endovascular device. According to some such embodiments, the length of segment 110 of the endovascular device is at least 122.4% of the length of the working segment 120 of the endovascular device. According to some such embodiments, the length of segment 110 of the endovascular device is at least 122.5% of the length of the working segment 120 of the endovascular device. According to some such embodiments, the length of segment 110 of the endovascular device is at least 122.6% of the length of the working segment 120 of the endovascular device. According to some such embodiments, the length of segment 110 of the endovascular device is at least 122.7% of the length of the working segment 120 of the endovascular device. According to some such embodiments, the length of segment 110 of the endovascular device is at least 122.8% of the length of the working segment 120 of the endovascular device. According to some such embodiments, the length of segment 110 of the endovascular device is at least 122.9% of the length of the working segment 120 of the endovascular device. According to some such embodiments, the length of segment 110 of the endovascular device is at least 123% of the length of the working segment 120 of the endovascular device. According to some such embodiments, the length of segment 110 of the endovascular device is at least 123.1% of the length of the working segment 120 of the endovascular device. According to some such embodiments, the length of segment 110 of the endovascular device is at least 123.2% of the length of the working segment 120 of the endovascular device. According to some such embodiments, the length of segment 110 of the endovascular device is at least 123.3% of the length of the working segment 120 of the endovascular device. According to some such embodiments, the length of segment 110 of the endovascular device is at least 123.4% of the length of the working segment 120 of the endovascular device. According to some such embodiments, the length of segment 110 of the endovascular device is at least 123.5% of the length of the working segment 120 of the endovascular device. According to some such embodiments, the length of segment 110 of the endovascular device is at least 123.6% of the length of the working segment 120 of the endovascular device. According to some such embodiments, the length of segment 110 of the endovascular device is at least 123.7% of the length of the working segment 120 of the endovascular device. According to some such embodiments, the length of segment 110 of the endovascular device is at least 123.8% of the length of the working segment 120 of the endovascular device. According to some such embodiments, the length of segment 110 of the endovascular device is at least 123.9% of the length of the working segment 120 of the endovascular device. According to some such embodiments, the length of segment 110 of the endovascular device is at least 124% of the length of the working segment 120 of the endovascular device. According to some such embodiments, the length of segment 110 of the endovascular device is at least 124.1% of the length of the working segment 120 of the endovascular device. According to some such embodiments, the length of segment 110 of the endovascular device is at least 124.2% of the length of the working segment 120 of the endovascular device. According to some such embodiments, the length of segment 110 of the endovascular device is at least 124.3% of the length of the working segment 120 of the endovascular device. According to some such embodiments, the length of segment 110 of the endovascular device is at least 124.4% of the length of the working segment 120 of the endovascular device. According to some such embodiments, the length of segment 110 of the endovascular device is at least 124.5% of the length of the working segment 120 of the endovascular device. According to some such embodiments, the length of segment 110 of the endovascular device is at least 124.6% of the length of the working segment 120 of the endovascular device. According to some such embodiments, the length of segment 110 of the endovascular device is at least 124.7% of the length of the working segment 120 of the endovascular device. According to some such embodiments, the length of segment 110 of the endovascular device is at least 124.8% of the length of the working segment 120 of the endovascular device. According to some such embodiments, the length of segment 110 of the endovascular device is at least 124.9% of the length of the working segment 120 of the endovascular device. According to some such embodiments, the length of segment 110 of the endovascular device is at least 125% of the length of the working segment 120 of the endovascular device.

According to some such embodiments, the length of segment 110 of the endovascular device is at least 125.1% of the length of the working segment 120 of the endovascular device. According to some such embodiments, the length of segment 110 of the endovascular device is at least 125.2% of the length of the working segment 120 of the endovascular device. According to some such embodiments, the length of segment 110 of the endovascular device is at least 125.3% of the length of the working segment 120 of the endovascular device. According to some such embodiments, the length of segment 110 of the endovascular device is at least 125.4% of the length of the working segment 120 of the endovascular device. According to some such embodiments, the length of segment 110 of the endovascular device is at least 125.5% of the length of the working segment 120 of the endovascular device. According to some such embodiments, the length of segment 110 of the endovascular device is at least 125.6% of the length of the working segment 120 of the endovascular device. According to some such embodiments, the length of segment 110 of the endovascular device is at least 125.7% of the length of the working segment 120 of the endovascular device. According to some such embodiments, the length of segment 110 of the endovascular device is at least 125.8% of the length of the working segment 120 of the endovascular device. According to some such embodiments, the length of segment 110 of the endovascular device is at least 125.9% of the length of the working segment 120 of the endovascular device. According to some such embodiments, the length of segment 110 of the endovascular device is at least 126% of the length of the working segment 120 of the endovascular device. According to some such embodiments, the length of segment 110 of the endovascular device is at least 126.1% of the length of the working segment 120 of the endovascular device. According to some such embodiments, the length of segment 110 of the endovascular device is at least 126.2% of the length of the working segment 120 of the endovascular device. According to some such embodiments, the length of segment 110 of the endovascular device is at least 126.3% of the length of the working segment 120 of the endovascular device. According to some such embodiments, the length of segment 110 of the endovascular device is at least 126.4% of the length of the working segment 120 of the endovascular device. According to some such embodiments, the length of segment 110 of the endovascular device is at least 126.5% of the length of the working segment 120 of the endovascular device. According to some such embodiments, the length of segment 110 of the endovascular device is at least 126.6% of the length of the working segment 120 of the endovascular device. According to some such embodiments, the length of segment 110 of the endovascular device is at least 126.7% of the length of the working segment 120 of the endovascular device. According to some such embodiments, the length of segment 110 of the endovascular device is at least 126.8% of the length of the working segment 120 of the endovascular device. According to some such embodiments, the length of segment 110 of the endovascular device is at least 126.9% of the length of the working segment 120 of the endovascular device. According to some such embodiments, the length of segment 110 of the endovascular device is at least 127% of the length of the working segment 120 of the endovascular device. According to some such embodiments, the length of segment 110 of the endovascular device is at least 127.1% of the length of the working segment 120 of the endovascular device. According to some such embodiments, the length of segment 110 of the endovascular device is at least 127.2% of the length of the working segment 120 of the endovascular device. According to some such embodiments, the length of segment 110 of the endovascular device is at least 127.3% of the length of the working segment 120 of the endovascular device. According to some such embodiments, the length of segment 110 of the endovascular device is at least 127.4% of the length of the working segment 120 of the endovascular device. According to some such embodiments, the length of segment 110 of the endovascular device is at least 127.5% of the length of the working segment 120 of the endovascular device. According to some such embodiments, the length of segment 110 of the endovascular device is at least 127.6% of the length of the working segment 120 of the endovascular device. According to some such embodiments, the length of segment 110 of the endovascular device is at least 127.7% of the length of the working segment 120 of the endovascular device. According to some such embodiments, the length of segment 110 of the endovascular device is at least 127.8% of the length of the working segment 120 of the endovascular device. According to some such embodiments, the length of segment 110 of the endovascular device is at least 127.9% of the length of the working segment 120 of the endovascular device. According to some such embodiments, the length of segment 110 of the endovascular device is at least 128% of the length of the working segment 120 of the endovascular device. According to some such embodiments, the length of segment 110 of the endovascular device is at least 128.1% of the length of the working segment 120 of the endovascular device. According to some such embodiments, the length of segment 110 of the endovascular device is at least 128.2% of the length of the working segment 120 of the endovascular device. According to some such embodiments, the length of segment 110 of the endovascular device is at least 128.3% of the length of the working segment 120 of the endovascular device. According to some such embodiments, the length of segment 110 of the endovascular device is at least 128.4% of the length of the working segment 120 of the endovascular device. According to some such embodiments, the length of segment 110 of the endovascular device is at least 128.5% of the length of the working segment 120 of the endovascular device. According to some such embodiments, the length of segment 110 of the endovascular device is at least 128.6% of the length of the working segment 120 of the endovascular device. According to some such embodiments, the length of segment 110 of the endovascular device is at least 128.7% of the length of the working segment 120 of the endovascular device. According to some such embodiments, the length of segment 110 of the endovascular device is at least 128.8% of the length of the working segment 120 of the endovascular device. According to some such embodiments, the length of segment 110 of the endovascular device is at least 128.9% of the length of the working segment 120 of the endovascular device. According to some such embodiments, the length of segment 110 of the endovascular device is at least 129% of the length of the working segment 120 of the endovascular device. According to some such embodiments, the length of segment 110 of the endovascular device is at least 129.1% of the length of the working segment 120 of the endovascular device. According to some such embodiments, the length of segment 110 of the endovascular device is at least 129.2% of the length of the working segment 120 of the endovascular device. According to some such embodiments, the length of segment 110 of the endovascular device is at least 129.3% of the length of the working segment 120 of the endovascular device. According to some such embodiments, the length of segment 110 of the endovascular device is at least 129.4% of the length of the working segment 120 of the endovascular device. According to some such embodiments, the length of segment 110 of the endovascular device is at least 129.5% of the length of the working segment 120 of the endovascular device. According to some such embodiments, the length of segment 110 of the endovascular device is at least 129.6% of the length of the working segment 120 of the endovascular device. According to some such embodiments, the length of segment 110 of the endovascular device is at least 129.7% of the length of the working segment 120 of the endovascular device. According to some such embodiments, the length of segment 110 of the endovascular device is at least 129.8% of the length of the working segment 120 of the endovascular device. According to some such embodiments, the length of segment 110 of the endovascular device is at least 129.9% of the length of the working segment 120 of the endovascular device. According to some such embodiments, the length of segment 110 of the endovascular device is at least 130% of the length of the working segment 120 of the endovascular device. According to some such embodiments, the length of segment 110 of the endovascular device is at least 130.1% of the length of the working segment 120 of the endovascular device. According to some such embodiments, the length of segment 110 of the endovascular device is at least 130.2% of the length of the working segment 120 of the endovascular device. According to some such embodiments, the length of segment 110 of the endovascular device is at least 130.3% of the length of the working segment 120 of the endovascular device. According to some such embodiments, the length of segment 110 of the endovascular device is at least 130.4% of the length of the working segment 120 of the endovascular device. According to some such embodiments, the length of segment 110 of the endovascular device is at least 130.5% of the length of the working segment 120 of the endovascular device. According to some such embodiments, the length of segment 110 of the endovascular device is at least 130.6% of the length of the working segment 120 of the endovascular device. According to some such embodiments, the length of segment 110 of the endovascular device is at least 130.7% of the length of the working segment 120 of the endovascular device. According to some such embodiments, the length of segment 110 of the endovascular device is at least 130.8% of the length of the working segment 120 of the endovascular device. According to some such embodiments, the length of segment 110 of the endovascular device is at least 130.9% of the length of the working segment 120 of the endovascular device. According to some such embodiments, the length of segment 110 of the endovascular device is at least 131% of the length of the working segment 120 of the endovascular device. According to some such embodiments, the length of segment 110 of the endovascular device is at least 131.1% of the length of the working segment 120 of the endovascular device. According to some such embodiments, the length of segment 110 of the endovascular device is at least 131.2% of the length of the working segment 120 of the endovascular device. According to some such embodiments, the length of segment 110 of the endovascular device is at least 131.3% of the length of the working segment 120 of the endovascular device. According to some such embodiments, the length of segment 110 of the endovascular device is at least 131.4% of the length of the working segment 120 of the endovascular device. According to some such embodiments, the length of segment 110 of the endovascular device is at least 131.5% of the length of the working segment 120 of the endovascular device. According to some such embodiments, the length of segment 110 of the endovascular device is at least 131.6% of the length of the working segment 120 of the endovascular device. According to some such embodiments, the length of segment 110 of the endovascular device is at least 131.7% of the length of the working segment 120 of the endovascular device. According to some such embodiments, the length of segment 110 of the endovascular device is at least 131.8% of the length of the working segment 120 of the endovascular device. According to some such embodiments, the length of segment 110 of the endovascular device is at least 131.9% of the length of the working segment 120 of the endovascular device. According to some such embodiments, the length of segment 110 of the endovascular device is at least 132% of the length of the working segment 120 of the endovascular device. According to some such embodiments, the length of segment 110 of the endovascular device is at least 132.1% of the length of the working segment 120 of the endovascular device. According to some such embodiments, the length of segment 110 of the endovascular device is at least 132.2% of the length of the working segment 120 of the endovascular device. According to some such embodiments, the length of segment 110 of the endovascular device is at least 132.3% of the length of the working segment 120 of the endovascular device. According to some such embodiments, the length of segment 110 of the endovascular device is at least 132.4% of the length of the working segment 120 of the endovascular device. According to some such embodiments, the length of segment 110 of the endovascular device is at least 132.5% of the length of the working segment 120 of the endovascular device. According to some such embodiments, the length of segment 110 of the endovascular device is at least 132.6% of the length of the working segment 120 of the endovascular device. According to some such embodiments, the length of segment 110 of the endovascular device is at least 132.7% of the length of the working segment 120 of the endovascular device. According to some such embodiments, the length of segment 110 of the endovascular device is at least 132.8% of the length of the working segment 120 of the endovascular device. According to some such embodiments, the length of segment 110 of the endovascular device is at least 132.9% of the length of the working segment 120 of the endovascular device. According to some such embodiments, the length of segment 110 of the endovascular device is at least 132% of the length of the working segment 120 of the endovascular device. According to some such embodiments, the length of segment 110 of the endovascular device is at least 133.1% of the length of the working segment 120 of the endovascular device. According to some such embodiments, the length of segment 110 of the endovascular device is at least 133.2% of the length of the working segment 120 of the endovascular device. According to some such embodiments, the length of segment 110 of the endovascular device is at least 133.3% of the length of the working segment 120 of the endovascular device. According to some such embodiments, the length of segment 110 of the endovascular device is at least 133.4% of the length of the working segment 120 of the endovascular device. According to some such embodiments, the length of segment 110 of the endovascular device is at least 133.5% of the length of the working segment 120 of the endovascular device. According to some such embodiments, the length of segment 110 of the endovascular device is at least 133.6% of the length of the working segment 120 of the endovascular device. According to some such embodiments, the length of segment 110 of the endovascular device is at least 133.7% of the length of the working segment 120 of the endovascular device. According to some such embodiments, the length of segment 110 of the endovascular device is at least 133.8% of the length of the working segment 120 of the endovascular device. According to some such embodiments, the length of segment 110 of the endovascular device is at least 133.9% of the length of the working segment 120 of the endovascular device. According to some such embodiments, the length of segment 110 of the endovascular device is at least 134% of the length of the working segment 120 of the endovascular device. According to some such embodiments, the length of segment 110 of the endovascular device is at least 134.1% of the length of the working segment 120 of the endovascular device. According to some such embodiments, the length of segment 110 of the endovascular device is at least 134.2% of the length of the working segment 120 of the endovascular device. According to some such embodiments, the length of segment 110 of the endovascular device is at least 134.3% of the length of the working segment 120 of the endovascular device. According to some such embodiments, the length of segment 110 of the endovascular device is at least 134.4% of the length of the working segment 120 of the endovascular device. According to some such embodiments, the length of segment 110 of the endovascular device is at least 134.5% of the length of the working segment 120 of the endovascular device. According to some such embodiments, the length of segment 110 of the endovascular device is at least 134.6% of the length of the working segment 120 of the endovascular device. According to some such embodiments, the length of segment 110 of the endovascular device is at least 134.7% of the length of the working segment 120 of the endovascular device. According to some such embodiments, the length of segment 110 of the endovascular device is at least 134.8% of the length of the working segment 120 of the endovascular device. According to some such embodiments, the length of segment 110 of the endovascular device is at least 134.9% of the length of the working segment 120 of the endovascular device. According to some such embodiments, the length of segment 110 of the endovascular device is at least 135% of the length of the working segment 120 of the endovascular device. According to some such embodiments, the length of segment 110 of the endovascular device is at least 135.1% of the length of the working segment 120 of the endovascular device. According to some such embodiments, the length of segment 110 of the endovascular device is at least 135.2% of the length of the working segment 120 of the endovascular device. According to some such embodiments, the length of segment 110 of the endovascular device is at least 135.3% of the length of the working segment 120 of the endovascular device. According to some such embodiments, the length of segment 110 of the endovascular device is at least 135.4% of the length of the working segment 120 of the endovascular device. According to some such embodiments, the length of segment 110 of the endovascular device is at least 135.5% of the length of the working segment 120 of the endovascular device. According to some such embodiments, the length of segment 110 of the endovascular device is at least 135.6% of the length of the working segment 120 of the endovascular device. According to some such embodiments, the length of segment 110 of the endovascular device is at least 135.7% of the length of the working segment 120 of the endovascular device. According to some such embodiments, the length of segment 110 of the endovascular device is at least 135.8% of the length of the working segment 120 of the endovascular device. According to some such embodiments, the length of segment 110 of the endovascular device is at least 135.9% of the length of the working segment 120 of the endovascular device. According to some such embodiments, the length of segment 110 of the endovascular device is at least 136% of the length of the working segment 120 of the endovascular device. According to some such embodiments, the length of segment 110 of the endovascular device is at least 136.1% of the length of the working segment 120 of the endovascular device. According to some such embodiments, the length of segment 110 of the endovascular device is at least 136.2% of the length of the working segment 120 of the endovascular device. According to some such embodiments, the length of segment 110 of the endovascular device is at least 136.3% of the length of the working segment 120 of the endovascular device. According to some such embodiments, the length of segment 110 of the endovascular device is at least 136.4% of the length of the working segment 120 of the endovascular device. According to some such embodiments, the length of segment 110 of the endovascular device is at least 136.5% of the length of the working segment 120 of the endovascular device. According to some such embodiments, the length of segment 110 of the endovascular device is at least 136.6% of the length of the working segment 120 of the endovascular device. According to some such embodiments, the length of segment 110 of the endovascular device is at least 136.7% of the length of the working segment 120 of the endovascular device. According to some such embodiments, the length of segment 110 of the endovascular device is at least 136.8% of the length of the working segment 120 of the endovascular device. According to some such embodiments, the length of segment 110 of the endovascular device is at least 136.9% of the length of the working segment 120 of the endovascular device. According to some such embodiments, the length of segment 110 of the endovascular device is at least 137% of the length of the working segment 120 of the endovascular device. According to some such embodiments, the length of segment 110 of the endovascular device is at least 137.1% of the length of the working segment 120 of the endovascular device. According to some such embodiments, the length of segment 110 of the endovascular device is at least 137.2% of the length of the working segment 120 of the endovascular device. According to some such embodiments, the length of segment 110 of the endovascular device is at least 137.3% of the length of the working segment 120 of the endovascular device. According to some such embodiments, the length of segment 110 of the endovascular device is at least 137.4% of the length of the working segment 120 of the endovascular device. According to some such embodiments, the length of segment 110 of the endovascular device is at least 137.5% of the length of the working segment 120 of the endovascular device. According to some such embodiments, the length of segment 110 of the endovascular device is at least 137.6% of the length of the working segment 120 of the endovascular device. According to some such embodiments, the length of segment 110 of the endovascular device is at least 137.7% of the length of the working segment 120 of the endovascular device. According to some such embodiments, the length of segment 110 of the endovascular device is at least 137.8% of the length of the working segment 120 of the endovascular device. According to some such embodiments, the length of segment 110 of the endovascular device is at least 137.9% of the length of the working segment 120 of the endovascular device. According to some such embodiments, the length of segment 110 of the endovascular device is at least 138% of the length of the working segment 120 of the endovascular device. According to some such embodiments, the length of segment 110 of the endovascular device is at least 138.1% of the length of the working segment 120 of the endovascular device. According to some such embodiments, the length of segment 110 of the endovascular device is at least 138.2% of the length of the working segment 120 of the endovascular device. According to some such embodiments, the length of segment 110 of the endovascular device is at least 138.3% of the length of the working segment 120 of the endovascular device. According to some such embodiments, the length of segment 110 of the endovascular device is at least 138.4% of the length of the working segment 120 of the endovascular device. According to some such embodiments, the length of segment 110 of the endovascular device is at least 138.5% of the length of the working segment 120 of the endovascular device. According to some such embodiments, the length of segment 110 of the endovascular device is at least 138.6% of the length of the working segment 120 of the endovascular device. According to some such embodiments, the length of segment 110 of the endovascular device is at least 138.7% of the length of the working segment 120 of the endovascular device. According to some such embodiments, the length of segment 110 of the endovascular device is at least 138.8% of the length of the working segment 120 of the endovascular device. According to some such embodiments, the length of segment 110 of the endovascular device is at least 138.9% of the length of the working segment 120 of the endovascular device. According to some such embodiments, the length of segment 110 of the endovascular device is at least 139% of the length of the working segment 120 of the endovascular device. According to some such embodiments, the length of segment 110 of the endovascular device is at least 139.1% of the length of the working segment 120 of the endovascular device. According to some such embodiments, the length of segment 110 of the endovascular device is at least 139.2% of the length of the working segment 120 of the endovascular device. According to some such embodiments, the length of segment 110 of the endovascular device is at least 139.3% of the length of the working segment 120 of the endovascular device. According to some such embodiments, the length of segment 110 of the endovascular device is at least 139.4% of the length of the working segment 120 of the endovascular device. According to some such embodiments, the length of segment 110 of the endovascular device is at least 139.5% of the length of the working segment 120 of the endovascular device. According to some such embodiments, the length of segment 110 of the endovascular device is at least 139.6% of the length of the working segment 120 of the endovascular device. According to some such embodiments, the length of segment 110 of the endovascular device is at least 139.7% of the length of the working segment 120 of the endovascular device. According to some such embodiments, the length of segment 110 of the endovascular device is at least 139.8% of the length of the working segment 120 of the endovascular device. According to some such embodiments, the length of segment 110 of the endovascular device is at least 139.9% of the length of the working segment 120 of the endovascular device.

According to some such embodiments, the length of segment 110 of the endovascular device is at least 140% of the length of the working segment 120 of the endovascular device. According to some such embodiments, the length of segment 110 of the endovascular device is at least 140.1% of the length of the working segment 120 of the endovascular device. According to some such embodiments, the length of segment 110 of the endovascular device is at least 140.2% of the length of the working segment 120 of the endovascular device. According to some such embodiments, the length of segment 110 of the endovascular device is at least 140.3% of the length of the working segment 120 of the endovascular device. According to some such embodiments, the length of segment 110 of the endovascular device is at least 140.4% of the length of the working segment 120 of the endovascular device. According to some such embodiments, the length of segment 110 of the endovascular device is at least 140.5% of the length of the working segment 120 of the endovascular device. According to some such embodiments, the length of segment 110 of the endovascular device is at least 140.6% of the length of the working segment 120 of the endovascular device. According to some such embodiments, the length of segment 110 of the endovascular device is at least 140.7% of the length of the working segment 120 of the endovascular device. According to some such embodiments, the length of segment 110 of the endovascular device is at least 140.8% of the length of the working segment 120 of the endovascular device. According to some such embodiments, the length of segment 110 of the endovascular device is at least 140.9% of the length of the working segment 120 of the endovascular device. According to some such embodiments, the length of segment 110 of the endovascular device is at least 141% of the length of the working segment 120 of the endovascular device. According to some such embodiments, the length of segment 110 of the endovascular device is at least 141.1% of the length of the working segment 120 of the endovascular device. According to some such embodiments, the length of segment 110 of the endovascular device is at least 141.2% of the length of the working segment 120 of the endovascular device. According to some such embodiments, the length of segment 110 of the endovascular device is at least 141.3% of the length of the working segment 120 of the endovascular device. According to some such embodiments, the length of segment 110 of the endovascular device is at least 141.4% of the length of the working segment 120 of the endovascular device. According to some such embodiments, the length of segment 110 of the endovascular device is at least 141.5% of the length of the working segment 120 of the endovascular device. According to some such embodiments, the length of segment 110 of the endovascular device is at least 141.6% of the length of the working segment 120 of the endovascular device. According to some such embodiments, the length of segment 110 of the endovascular device is at least 141.7% of the length of the working segment 120 of the endovascular device. According to some such embodiments, the length of segment 110 of the endovascular device is at least 141.8% of the length of the working segment 120 of the endovascular device. According to some such embodiments, the length of segment 110 of the endovascular device is at least 141.9% of the length of the working segment 120 of the endovascular device. According to some such embodiments, the length of segment 110 of the endovascular device is at least 142% of the length of the working segment 120 of the endovascular device. According to some such embodiments, the length of segment 110 of the endovascular device is at least 142.1% of the length of the working segment 120 of the endovascular device. According to some such embodiments, the length of segment 110 of the endovascular device is at least 142.2% of the length of the working segment 120 of the endovascular device. According to some such embodiments, the length of segment 110 of the endovascular device is at least 142.3% of the length of the working segment 120 of the endovascular device. According to some such embodiments, the length of segment 110 of the endovascular device is at least 142.4% of the length of the working segment 120 of the endovascular device. According to some such embodiments, the length of segment 110 of the endovascular device is at least 142.5% of the length of the working segment 120 of the endovascular device. According to some such embodiments, the length of segment 110 of the endovascular device is at least 142.6% of the length of the working segment 120 of the endovascular device. According to some such embodiments, the length of segment 110 of the endovascular device is at least 142.7% of the length of the working segment 120 of the endovascular device. According to some such embodiments, the length of segment 110 of the endovascular device is at least 142.8% of the length of the working segment 120 of the endovascular device. According to some such embodiments, the length of segment 110 of the endovascular device is at least 142.9% of the length of the working segment 120 of the endovascular device. According to some such embodiments, the length of segment 110 of the endovascular device is at least 143% of the length of the working segment 120 of the endovascular device. According to some such embodiments, the length of segment 110 of the endovascular device is at least 143.1% of the length of the working segment 120 of the endovascular device. According to some such embodiments, the length of segment 110 of the endovascular device is at least 143.2% of the length of the working segment 120 of the endovascular device. According to some such embodiments, the length of segment 110 of the endovascular device is at least 143.3% of the length of the working segment 120 of the endovascular device. According to some such embodiments, the length of segment 110 of the endovascular device is at least 143.4% of the length of the working segment 120 of the endovascular device. According to some such embodiments, the length of segment 110 of the endovascular device is at least 143.5% of the length of the working segment 120 of the endovascular device. According to some such embodiments, the length of segment 110 of the endovascular device is at least 143.6% of the length of the working segment 120 of the endovascular device. According to some such embodiments, the length of segment 110 of the endovascular device is at least 143.7% of the length of the working segment 120 of the endovascular device. According to some such embodiments, the length of segment 110 of the endovascular device is at least 143.8% of the length of the working segment 120 of the endovascular device. According to some such embodiments, the length of segment 110 of the endovascular device is at least 143.9% of the length of the working segment 120 of the endovascular device. According to some such embodiments, the length of segment 110 of the endovascular device is at least 144% of the length of the working segment 120 of the endovascular device. According to some such embodiments, the length of segment 110 of the endovascular device is at least 144.1% of the length of the working segment 120 of the endovascular device. According to some such embodiments, the length of segment 110 of the endovascular device is at least 144.2% of the length of the working segment 120 of the endovascular device. According to some such embodiments, the length of segment 110 of the endovascular device is at least 144.3% of the length of the working segment 120 of the endovascular device. According to some such embodiments, the length of segment 110 of the endovascular device is at least 144.4% of the length of the working segment 120 of the endovascular device. According to some such embodiments, the length of segment 110 of the endovascular device is at least 144.5% of the length of the working segment 120 of the endovascular device. According to some such embodiments, the length of segment 110 of the endovascular device is at least 144.6% of the length of the working segment 120 of the endovascular device. According to some such embodiments, the length of segment 110 of the endovascular device is at least 144.7% of the length of the working segment 120 of the endovascular device. According to some such embodiments, the length of segment 110 of the endovascular device is at least 144.8% of the length of the working segment 120 of the endovascular device. According to some such embodiments, the length of segment 110 of the endovascular device is at least 144.9% of the length of the working segment 120 of the endovascular device. According to some such embodiments, the length of segment 110 of the endovascular device is at least 145% of the length of the working segment 120 of the endovascular device. According to some such embodiments, the length of segment 110 of the endovascular device is at least 145.1% of the length of the working segment 120 of the endovascular device. According to some such embodiments, the length of segment 110 of the endovascular device is at least 145.2% of the length of the working segment 120 of the endovascular device. According to some such embodiments, the length of segment 110 of the endovascular device is at least 145.3% of the length of the working segment 120 of the endovascular device. According to some such embodiments, the length of segment 110 of the endovascular device is at least 145.4% of the length of the working segment 120 of the endovascular device. According to some such embodiments, the length of segment 110 of the endovascular device is at least 145.5% of the length of the working segment 120 of the endovascular device. According to some such embodiments, the length of segment 110 of the endovascular device is at least 145.6% of the length of the working segment 120 of the endovascular device. According to some such embodiments, the length of segment 110 of the endovascular device is at least 145.7% of the length of the working segment 120 of the endovascular device. According to some such embodiments, the length of segment 110 of the endovascular device is at least 145.8% of the length of the working segment 120 of the endovascular device. According to some such embodiments, the length of segment 110 of the endovascular device is at least 145.9% of the length of the working segment 120 of the endovascular device. According to some such embodiments, the length of segment 110 of the endovascular device is at least 146% of the length of the working segment 120 of the endovascular device. According to some such embodiments, the length of segment 110 of the endovascular device is at least 146.1% of the length of the working segment 120 of the endovascular device. According to some such embodiments, the length of segment 110 of the endovascular device is at least 146.2% of the length of the working segment 120 of the endovascular device. According to some such embodiments, the length of segment 110 of the endovascular device is at least 146.3% of the length of the working segment 120 of the endovascular device. According to some such embodiments, the length of segment 110 of the endovascular device is at least 146.4% of the length of the working segment 120 of the endovascular device. According to some such embodiments, the length of segment 110 of the endovascular device is at least 146.5% of the length of the working segment 120 of the endovascular device. According to some such embodiments, the length of segment 110 of the endovascular device is at least 146.6% of the length of the working segment 120 of the endovascular device. According to some such embodiments, the length of segment 110 of the endovascular device is at least 146.7% of the length of the working segment 120 of the endovascular device. According to some such embodiments, the length of segment 110 of the endovascular device is at least 146.8% of the length of the working segment 120 of the endovascular device. According to some such embodiments, the length of segment 110 of the endovascular device is at least 146.9% of the length of the working segment 120 of the endovascular device. According to some such embodiments, the length of segment 110 of the endovascular device is at least 147% of the length of the working segment 120 of the endovascular device. According to some such embodiments, the length of segment 110 of the endovascular device is at least 147.1% of the length of the working segment 120 of the endovascular device. According to some such embodiments, the length of segment 110 of the endovascular device is at least 147.2% of the length of the working segment 120 of the endovascular device. According to some such embodiments, the length of segment 110 of the endovascular device is at least 147.3% of the length of the working segment 120 of the endovascular device. According to some such embodiments, the length of segment 110 of the endovascular device is at least 147.4% of the length of the working segment 120 of the endovascular device. According to some such embodiments, the length of segment 110 of the endovascular device is at least 147.5% of the length of the working segment 120 of the endovascular device. According to some such embodiments, the length of segment 110 of the endovascular device is at least 147.6% of the length of the working segment 120 of the endovascular device. According to some such embodiments, the length of segment 110 of the endovascular device is at least 147.7% of the length of the working segment 120 of the endovascular device. According to some such embodiments, the length of segment 110 of the endovascular device is at least 147.8% of the length of the working segment 120 of the endovascular device. According to some such embodiments, the length of segment 110 of the endovascular device is at least 147.9% of the length of the working segment 120 of the endovascular device. According to some such embodiments, the length of segment 110 of the endovascular device is at least 148% of the length of the working segment 120 of the endovascular device. According to some such embodiments, the length of segment 110 of the endovascular device is at least 148.1% of the length of the working segment 120 of the endovascular device. According to some such embodiments, the length of segment 110 of the endovascular device is at least 148.2% of the length of the working segment 120 of the endovascular device. According to some such embodiments, the length of segment 110 of the endovascular device is at least 148.3% of the length of the working segment 120 of the endovascular device. According to some such embodiments, the length of segment 110 of the endovascular device is at least 148.4% of the length of the working segment 120 of the endovascular device. According to some such embodiments, the length of segment 110 of the endovascular device is at least 148.5% of the length of the working segment 120 of the endovascular device. According to some such embodiments, the length of segment 110 of the endovascular device is at least 148.6% of the length of the working segment 120 of the endovascular device. According to some such embodiments, the length of segment 110 of the endovascular device is at least 148.7% of the length of the working segment 120 of the endovascular device. According to some such embodiments, the length of segment 110 of the endovascular device is at least 148.8% of the length of the working segment 120 of the endovascular device. According to some such embodiments, the length of segment 110 of the endovascular device is at least 148.9% of the length of the working segment 120 of the endovascular device. According to some such embodiments, the length of segment 110 of the endovascular device is at least 149% of the length of the working segment 120 of the endovascular device. According to some such embodiments, the length of segment 110 of the endovascular device is at least 149.1% of the length of the working segment 120 of the endovascular device. According to some such embodiments, the length of segment 110 of the endovascular device is at least 149.2% of the length of the working segment 120 of the endovascular device. According to some such embodiments, the length of segment 110 of the endovascular device is at least 149.3% of the length of the working segment 120 of the endovascular device. According to some such embodiments, the length of segment 110 of the endovascular device is at least 149.4% of the length of the working segment 120 of the endovascular device. According to some such embodiments, the length of segment 110 of the endovascular device is at least 149.5% of the length of the working segment 120 of the endovascular device. According to some such embodiments, the length of segment 110 of the endovascular device is at least 149.6% of the length of the working segment 120 of the endovascular device. According to some such embodiments, the length of segment 110 of the endovascular device is at least 149.7% of the length of the working segment 120 of the endovascular device. According to some such embodiments, the length of segment 110 of the endovascular device is at least 149.8% of the length of the working segment 120 of the endovascular device. According to some such embodiments, the length of segment 110 of the endovascular device is at least 149.9% of the length of the working segment 120 of the endovascular device. According to some such embodiments, the length of segment 110 of the endovascular device is at least 150% of the length of the working segment 120 of the endovascular device. According to some such embodiments, the length of segment 110 of the endovascular device is at least 150.1% of the length of the working segment 120 of the endovascular device. According to some such embodiments, the length of segment 110 of the endovascular device is at least 150.2% of the length of the working segment 120 of the endovascular device. According to some such embodiments, the length of segment 110 of the endovascular device is at least 150.3% of the length of the working segment 120 of the endovascular device. According to some such embodiments, the length of segment 110 of the endovascular device is at least 150.4% of the length of the working segment 120 of the endovascular device. According to some such embodiments, the length of segment 110 of the endovascular device is at least 150.5% of the length of the working segment 120 of the endovascular device. According to some such embodiments, the length of segment 110 of the endovascular device is at least 150.6% of the length of the working segment 120 of the endovascular device. According to some such embodiments, the length of segment 110 of the endovascular device is at least 150.7% of the length of the working segment 120 of the endovascular device. According to some such embodiments, the length of segment 110 of the endovascular device is at least 150.8% of the length of the working segment 120 of the endovascular device. According to some such embodiments, the length of segment 110 of the endovascular device is at least 150.9% of the length of the working segment 120 of the endovascular device. According to some such embodiments, the length of segment 110 of the endovascular device is at least 151% of the length of the working segment 120 of the endovascular device. According to some such embodiments, the length of segment 110 of the endovascular device is at least 151.1% of the length of the working segment 120 of the endovascular device. According to some such embodiments, the length of segment 110 of the endovascular device is at least 151.2% of the length of the working segment 120 of the endovascular device. According to some such embodiments, the length of segment 110 of the endovascular device is at least 151.3% of the length of the working segment 120 of the endovascular device. According to some such embodiments, the length of segment 110 of the endovascular device is at least 151.4% of the length of the working segment 120 of the endovascular device. According to some such embodiments, the length of segment 110 of the endovascular device is at least 151.5% of the length of the working segment 120 of the endovascular device. According to some such embodiments, the length of segment 110 of the endovascular device is at least 151.6% of the length of the working segment 120 of the endovascular device. According to some such embodiments, the length of segment 110 of the endovascular device is at least 151.7% of the length of the working segment 120 of the endovascular device. According to some such embodiments, the length of segment 110 of the endovascular device is at least 151.8% of the length of the working segment 120 of the endovascular device. According to some such embodiments, the length of segment 110 of the endovascular device is at least 151.9% of the length of the working segment 120 of the endovascular device. According to some such embodiments, the length of segment 110 of the endovascular device is at least 152% of the length of the working segment 120 of the endovascular device. According to some such embodiments, the length of segment 110 of the endovascular device is at least 152.1% of the length of the working segment 120 of the endovascular device. According to some such embodiments, the length of segment 110 of the endovascular device is at least 152.2% of the length of the working segment 120 of the endovascular device. According to some such embodiments, the length of segment 110 of the endovascular device is at least 152.3% of the length of the working segment 120 of the endovascular device. According to some such embodiments, the length of segment 110 of the endovascular device is at least 152.4% of the length of the working segment 120 of the endovascular device. According to some such embodiments, the length of segment 110 of the endovascular device is at least 152.5% of the length of the working segment 120 of the endovascular device. According to some such embodiments, the length of segment 110 of the endovascular device is at least 152.6% of the length of the working segment 120 of the endovascular device. According to some such embodiments, the length of segment 110 of the endovascular device is at least 152.7% of the length of the working segment 120 of the endovascular device. According to some such embodiments, the length of segment 110 of the endovascular device is at least 152.8% of the length of the working segment 120 of the endovascular device. According to some such embodiments, the length of segment 110 of the endovascular device is at least 152.9% of the length of the working segment 120 of the endovascular device. According to some such embodiments, the length of segment 110 of the endovascular device is at least 153% of the length of the working segment 120 of the endovascular device. According to some such embodiments, the length of segment 110 of the endovascular device is at least 153.1% of the length of the working segment 120 of the endovascular device. According to some such embodiments, the length of segment 110 of the endovascular device is at least 153.2% of the length of the working segment 120 of the endovascular device. According to some such embodiments, the length of segment 110 of the endovascular device is at least 153.3% of the length of the working segment 120 of the endovascular device. According to some such embodiments, the length of segment 110 of the endovascular device is at least 153.4% of the length of the working segment 120 of the endovascular device. According to some such embodiments, the length of segment 110 of the endovascular device is at least 153.5% of the length of the working segment 120 of the endovascular device. According to some such embodiments, the length of segment 110 of the endovascular device is at least 153.6% of the length of the working segment 120 of the endovascular device. According to some such embodiments, the length of segment 110 of the endovascular device is at least 153.7% of the length of the working segment 120 of the endovascular device. According to some such embodiments, the length of segment 110 of the endovascular device is at least 153.8% of the length of the working segment 120 of the endovascular device. According to some such embodiments, the length of segment 110 of the endovascular device is at least 153.9% of the length of the working segment 120 of the endovascular device. According to some such embodiments, the length of segment 110 of the endovascular device is at least 154% of the length of the working segment 120 of the endovascular device. According to some such embodiments, the length of segment 110 of the endovascular device is at least 154.1% of the length of the working segment 120 of the endovascular device. According to some such embodiments, the length of segment 110 of the endovascular device is at least 154.2% of the length of the working segment 120 of the endovascular device. According to some such embodiments, the length of segment 110 of the endovascular device is at least 154.3% of the length of the working segment 120 of the endovascular device. According to some such embodiments, the length of segment 110 of the endovascular device is at least 154.4% of the length of the working segment 120 of the endovascular device. According to some such embodiments, the length of segment 110 of the endovascular device is at least 154.5% of the length of the working segment 120 of the endovascular device. According to some such embodiments, the length of segment 110 of the endovascular device is at least 154.6% of the length of the working segment 120 of the endovascular device. According to some such embodiments, the length of segment 110 of the endovascular device is at least 154.7% of the length of the working segment 120 of the endovascular device. According to some such embodiments, the length of segment 110 of the endovascular device is at least 154.8% of the length of the working segment 120 of the endovascular device. According to some such embodiments, the length of segment 110 of the endovascular device is at least 154.9% of the length of the working segment 120 of the endovascular device. According to some such embodiments, the length of segment 110 of the endovascular device is at least 155% of the length of the working segment 120 of the endovascular device. According to some such embodiments, the length of segment 110 of the endovascular device is at least 155.1% of the length of the working segment 120 of the endovascular device. According to some such embodiments, the length of segment 110 of the endovascular device is at least 155.2% of the length of the working segment 120 of the endovascular device. According to some such embodiments, the length of segment 110 of the endovascular device is at least 155.3% of the length of the working segment 120 of the endovascular device. According to some such embodiments, the length of segment 110 of the endovascular device is at least 155.4% of the length of the working segment 120 of the endovascular device. According to some such embodiments, the length of segment 110 of the endovascular device is at least 155.5% of the length of the working segment 120 of the endovascular device. According to some such embodiments, the length of segment 110 of the endovascular device is at least 155.6% of the length of the working segment 120 of the endovascular device. According to some such embodiments, the length of segment 110 of the endovascular device is at least 155.7% of the length of the working segment 120 of the endovascular device. According to some such embodiments, the length of segment 110 of the endovascular device is at least 155.8% of the length of the working segment 120 of the endovascular device. According to some such embodiments, the length of segment 110 of the endovascular device is at least 155.9% of the length of the working segment 120 of the endovascular device. According to some such embodiments, the length of segment 110 of the endovascular device is at least 156% of the length of the working segment 120 of the endovascular device. According to some such embodiments, the length of segment 110 of the endovascular device is at least 156.1% of the length of the working segment 120 of the endovascular device. According to some such embodiments, the length of segment 110 of the endovascular device is at least 156.2% of the length of the working segment 120 of the endovascular device. According to some such embodiments, the length of segment 110 of the endovascular device is at least 156.3% of the length of the working segment 120 of the endovascular device. According to some such embodiments, the length of segment 110 of the endovascular device is at least 156.4% of the length of the working segment 120 of the endovascular device. According to some such embodiments, the length of segment 110 of the endovascular device is at least 156.5% of the length of the working segment 120 of the endovascular device. According to some such embodiments, the length of segment 110 of the endovascular device is at least 156.6% of the length of the working segment 120 of the endovascular device. According to some such embodiments, the length of segment 110 of the endovascular device is at least 156.7% of the length of the working segment 120 of the endovascular device. According to some such embodiments, the length of segment 110 of the endovascular device is at least 156.8% of the length of the working segment 120 of the endovascular device. According to some such embodiments, the length of segment 110 of the endovascular device is at least 156.9% of the length of the working segment 120 of the endovascular device. According to some such embodiments, the length of segment 110 of the endovascular device is at least 157% of the length of the working segment 120 of the endovascular device.

According to some such embodiments, the length of segment 110 of the endovascular device is at least 157.1% of the length of the working segment 120 of the endovascular device. According to some such embodiments, the length of segment 110 of the endovascular device is at least 157.2% of the length of the working segment 120 of the endovascular device. According to some such embodiments, the length of segment 110 of the endovascular device is at least 157.3% of the length of the working segment 120 of the endovascular device. According to some such embodiments, the length of segment 110 of the endovascular device is at least 157.4% of the length of the working segment 120 of the endovascular device. According to some such embodiments, the length of segment 110 of the endovascular device is at least 157.5% of the length of the working segment 120 of the endovascular device. According to some such embodiments, the length of segment 110 of the endovascular device is at least 157.6% of the length of the working segment 120 of the endovascular device. According to some such embodiments, the length of segment 110 of the endovascular device is at least 157.7% of the length of the working segment 120 of the endovascular device. According to some such embodiments, the length of segment 110 of the endovascular device is at least 157.8% of the length of the working segment 120 of the endovascular device. According to some such embodiments, the length of segment 110 of the endovascular device is at least 157.9% of the length of the working segment 120 of the endovascular device. According to some such embodiments, the length of segment 110 of the endovascular device is at least 158% of the length of the working segment 120 of the endovascular device. According to some such embodiments, the length of segment 110 of the endovascular device is at least 158.1% of the length of the working segment 120 of the endovascular device. According to some such embodiments, the length of segment 110 of the endovascular device is at least 158.2% of the length of the working segment 120 of the endovascular device. According to some such embodiments, the length of segment 110 of the endovascular device is at least 158.3% of the length of the working segment 120 of the endovascular device. According to some such embodiments, the length of segment 110 of the endovascular device is at least 158.4% of the length of the working segment 120 of the endovascular device. According to some such embodiments, the length of segment 110 of the endovascular device is at least 158.5% of the length of the working segment 120 of the endovascular device. According to some such embodiments, the length of segment 110 of the endovascular device is at least 158.6% of the length of the working segment 120 of the endovascular device. According to some such embodiments, the length of segment 110 of the endovascular device is at least 158.7% of the length of the working segment 120 of the endovascular device. According to some such embodiments, the length of segment 110 of the endovascular device is at least 158.8% of the length of the working segment 120 of the endovascular device. According to some such embodiments, the length of segment 110 of the endovascular device is at least 158.9% of the length of the working segment 120 of the endovascular device. According to some such embodiments, the length of segment 110 of the endovascular device is at least 159% of the length of the working segment 120 of the endovascular device. According to some such embodiments, the length of segment 110 of the endovascular device is at least 159.1% of the length of the working segment 120 of the endovascular device. According to some such embodiments, the length of segment 110 of the endovascular device is at least 159.2% of the length of the working segment 120 of the endovascular device. According to some such embodiments, the length of segment 110 of the endovascular device is at least 159.3% of the length of the working segment 120 of the endovascular device. According to some such embodiments, the length of segment 110 of the endovascular device is at least 159.4% of the length of the working segment 120 of the endovascular device. According to some such embodiments, the length of segment 110 of the endovascular device is at least 159.5% of the length of the working segment 120 of the endovascular device. According to some such embodiments, the length of segment 110 of the endovascular device is at least 159.6% of the length of the working segment 120 of the endovascular device. According to some such embodiments, the length of segment 110 of the endovascular device is at least 159.7% of the length of the working segment 120 of the endovascular device. According to some such embodiments, the length of segment 110 of the endovascular device is at least 159.8% of the length of the working segment 120 of the endovascular device. According to some such embodiments, the length of segment 110 of the endovascular device According to some such embodiments, the length of segment 110 of the endovascular device is at least 160% of the length of the working segment 120 of the endovascular device. According to some such embodiments, the length of segment 110 of the endovascular device is at least 160.1% of the length of the working segment 120 of the endovascular device. According to some such embodiments, the length of segment 110 of the endovascular device is at least 160.2% of the length of the working segment 120 of the endovascular device. According to some such embodiments, the length of segment 110 of the endovascular device is at least 160.3% of the length of the working segment 120 of the endovascular device. According to some such embodiments, the length of segment 110 of the endovascular device is at least 160.4% of the length of the working segment 120 of the endovascular device. According to some such embodiments, the length of segment 110 of the endovascular device is at least 160.5% of the length of the working segment 120 of the endovascular device. According to some such embodiments, the length of segment 110 of the endovascular device is at least 160.6% of the length of the working segment 120 of the endovascular device. According to some such embodiments, the length of segment 110 of the endovascular device is at least 160.7% of the length of the working segment 120 of the endovascular device. According to some such embodiments, the length of segment 110 of the endovascular device is at least 160.8% of the length of the working segment 120 of the endovascular device. According to some such embodiments, the length of segment 110 of the endovascular device is at least 160.9% of the length of the working segment 120 of the endovascular device. According to some such embodiments, the length of segment 110 of the endovascular device is at least 161% of the length of the working segment 120 of the endovascular device. According to some such embodiments, the length of segment 110 of the endovascular device is at least 161.1% of the length of the working segment 120 of the endovascular device. According to some such embodiments, the length of segment 110 of the endovascular device is at least 161.2% of the length of the working segment 120 of the endovascular device. According to some such embodiments, the length of segment 110 of the endovascular device is at least 161.3% of the length of the working segment 120 of the endovascular device. According to some such embodiments, the length of segment 110 of the endovascular device is at least 161.4% of the length of the working segment 120 of the endovascular device. According to some such embodiments, the length of segment 110 of the endovascular device is at least 161.5% of the length of the working segment 120 of the endovascular device. According to some such embodiments, the length of segment 110 of the endovascular device is at least 161.6% of the length of the working segment 120 of the endovascular device. According to some such embodiments, the length of segment 110 of the endovascular device is at least 161.7% of the length of the working segment 120 of the endovascular device. According to some such embodiments, the length of segment 110 of the endovascular device is at least 161.8% of the length of the working segment 120 of the endovascular device. According to some such embodiments, the length of segment 110 of the endovascular device is at least 161.9% of the length of the working segment 120 of the endovascular device. According to some such embodiments, the length of segment 110 of the endovascular device is at least 162% of the length of the working segment 120 of the endovascular device. According to some such embodiments, the length of segment 110 of the endovascular device is at least 162.1% of the length of the working segment 120 of the endovascular device. According to some such embodiments, the length of segment 110 of the endovascular device is at least 162.2% of the length of the working segment 120 of the endovascular device. According to some such embodiments, the length of segment 110 of the endovascular device is at least 162.3% of the length of the working segment 120 of the endovascular device. According to some such embodiments, the length of segment 110 of the endovascular device is at least 162.4% of the length of the working segment 120 of the endovascular device. According to some such embodiments, the length of segment 110 of the endovascular device is at least 162.5% of the length of the working segment 120 of the endovascular device. According to some such embodiments, the length of segment 110 of the endovascular device is at least 162.6% of the length of the working segment 120 of the endovascular device. According to some such embodiments, the length of segment 110 of the endovascular device is at least 162.7% of the length of the working segment 120 of the endovascular device. According to some such embodiments, the length of segment 110 of the endovascular device is at least 162.8% of the length of the working segment 120 of the endovascular device. According to some such embodiments, the length of segment 110 of the endovascular device is at least 162.9% of the length of the working segment 120 of the endovascular device. According to some such embodiments, the length of segment 110 of the endovascular device is at least 163% of the length of the working segment 120 of the endovascular device. According to some such embodiments, the length of segment 110 of the endovascular device is at least 163.1% of the length of the working segment 120 of the endovascular device. According to some such embodiments, the length of segment 110 of the endovascular device is at least 163.2% of the length of the working segment 120 of the endovascular device. According to some such embodiments, the length of segment 110 of the endovascular device is at least 163.3% of the length of the working segment 120 of the endovascular device. According to some such embodiments, the length of segment 110 of the endovascular device is at least 163.4% of the length of the working segment 120 of the endovascular device. According to some such embodiments, the length of segment 110 of the endovascular device is at least 163.5% of the length of the working segment 120 of the endovascular device. According to some such embodiments, the length of segment 110 of the endovascular device is at least 163.6% of the length of the working segment 120 of the endovascular device. According to some such embodiments, the length of segment 110 of the endovascular device is at least 163.7% of the length of the working segment 120 of the endovascular device. According to some such embodiments, the length of segment 110 of the endovascular device is at least 163.8% of the length of the working segment 120 of the endovascular device. According to some such embodiments, the length of segment 110 of the endovascular device is at least 163.9% of the length of the working segment 120 of the endovascular device. According to some such embodiments, the length of segment 110 of the endovascular device is at least 164% of the length of the working segment 120 of the endovascular device. According to some such embodiments, the length of segment 110 of the endovascular device is at least 164.1% of the length of the working segment 120 of the endovascular device. According to some such embodiments, the length of segment 110 of the endovascular device is at least 164.2% of the length of the working segment 120 of the endovascular device. According to some such embodiments, the length of segment 110 of the endovascular device is at least 164.3% of the length of the working segment 120 of the endovascular device. According to some such embodiments, the length of segment 110 of the endovascular device is at least 164.4% of the length of the working segment 120 of the endovascular device. According to some such embodiments, the length of segment 110 of the endovascular device is at least 164.5% of the length of the working segment 120 of the endovascular device. According to some such embodiments, the length of segment 110 of the endovascular device is at least 164.6% of the length of the working segment 120 of the endovascular device. According to some such embodiments, the length of segment 110 of the endovascular device is at least 164.7% of the length of the working segment 120 of the endovascular device. According to some such embodiments, the length of segment 110 of the endovascular device is at least 164.8% of the length of the working segment 120 of the endovascular device. According to some such embodiments, the length of segment 110 of the endovascular device is at least 164.9% of the length of the working segment 120 of the endovascular device. According to some such embodiments, the length of segment 110 of the endovascular device is at least 165% of the length of the working segment 120 of the endovascular device. According to some such embodiments, the length of segment 110 of the endovascular device is at least 165.1% of the length of the working segment 120 of the endovascular device. According to some such embodiments, the length of segment 110 of the endovascular device is at least 165.2% of the length of the working segment 120 of the endovascular device. According to some such embodiments, the length of segment 110 of the endovascular device is at least 165.3% of the length of the working segment 120 of the endovascular device. According to some such embodiments, the length of segment 110 of the endovascular device is at least 165.4% of the length of the working segment 120 of the endovascular device. According to some such embodiments, the length of segment 110 of the endovascular device is at least 165.5% of the length of the working segment 120 of the endovascular device. According to some such embodiments, the length of segment 110 of the endovascular device is at least 165.6% of the length of the working segment 120 of the endovascular device. According to some such embodiments, the length of segment 110 of the endovascular device is at least 165.7% of the length of the working segment 120 of the endovascular device. According to some such embodiments, the length of segment 110 of the endovascular device is at least 165.8% of the length of the working segment 120 of the endovascular device. According to some such embodiments, the length of segment 110 of the endovascular device is at least 165.9% of the length of the working segment 120 of the endovascular device. According to some such embodiments, the length of segment 110 of the endovascular device is at least 166% of the length of the working segment 120 of the endovascular device. According to some such embodiments, the length of segment 110 of the endovascular device is at least 166.1% of the length of the working segment 120 of the endovascular device. According to some such embodiments, the length of segment 110 of the endovascular device is at least 166.2% of the length of the working segment 120 of the endovascular device. According to some such embodiments, the length of segment 110 of the endovascular device is at least 166.3% of the length of the working segment 120 of the endovascular device. According to some such embodiments, the length of segment 110 of the endovascular device is at least 166.4% of the length of the working segment 120 of the endovascular device. According to some such embodiments, the length of segment 110 of the endovascular device is at least 166.5% of the length of the working segment 120 of the endovascular device. According to some such embodiments, the length of segment 110 of the endovascular device is at least 166.6% of the length of the working segment 120 of the endovascular device. According to some such embodiments, the length of segment 110 of the endovascular device is at least 166.7% of the length of the working segment 120 of the endovascular device. According to some such embodiments, the length of segment 110 of the endovascular device is at least 166.8% of the length of the working segment 120 of the endovascular device. According to some such embodiments, the length of segment 110 of the endovascular device is at least 166.9% of the length of the working segment 120 of the endovascular device. According to some such embodiments, the length of segment 110 of the endovascular device is at least 167% of the length of the working segment 120 of the endovascular device. According to some such embodiments, the length of segment 110 of the endovascular device is at least 167.1% of the length of the working segment 120 of the endovascular device. According to some such embodiments, the length of segment 110 of the endovascular device is at least 167.2% of the length of the working segment 120 of the endovascular device. According to some such embodiments, the length of segment 110 of the endovascular device is at least 167.3% of the length of the working segment 120 of the endovascular device. According to some such embodiments, the length of segment 110 of the endovascular device is at least 167.4% of the length of the working segment 120 of the endovascular device. According to some such embodiments, the length of segment 110 of the endovascular device is at least 167.5% of the length of the working segment 120 of the endovascular device. According to some such embodiments, the length of segment 110 of the endovascular device is at least 167.6% of the length of the working segment 120 of the endovascular device. According to some such embodiments, the length of segment 110 of the endovascular device is at least 167.7% of the length of the working segment 120 of the endovascular device. According to some such embodiments, the length of segment 110 of the endovascular device is at least 167.8% of the length of the working segment 120 of the endovascular device. According to some such embodiments, the length of segment 110 of the endovascular device is at least 167.9% of the length of the working segment 120 of the endovascular device. According to some such embodiments, the length of segment 110 of the endovascular device is at least 168% of the length of the working segment 120 of the endovascular device. According to some such embodiments, the length of segment 110 of the endovascular device is at least 168.1% of the length of the working segment 120 of the endovascular device. According to some such embodiments, the length of segment 110 of the endovascular device is at least 168.2% of the length of the working segment 120 of the endovascular device. According to some such embodiments, the length of segment 110 of the endovascular device is at least 168.3% of the length of the working segment 120 of the endovascular device. According to some such embodiments, the length of segment 110 of the endovascular device is at least 168.4% of the length of the working segment 120 of the endovascular device. According to some such embodiments, the length of segment 110 of the endovascular device is at least 168.5% of the length of the working segment 120 of the endovascular device. According to some such embodiments, the length of segment 110 of the endovascular device is at least 168.6% of the length of the working segment 120 of the endovascular device. According to some such embodiments, the length of segment 110 of the endovascular device is at least 168.7% of the length of the working segment 120 of the endovascular device. According to some such embodiments, the length of segment 110 of the endovascular device is at least 168.8% of the length of the working segment 120 of the endovascular device. According to some such embodiments, the length of segment 110 of the endovascular device is at least 168.9% of the length of the working segment 120 of the endovascular device. According to some such embodiments, the length of segment 110 of the endovascular device is at least 169% of the length of the working segment 120 of the endovascular device. According to some such embodiments, the length of segment 110 of the endovascular device is at least 169.1% of the length of the working segment 120 of the endovascular device. According to some such embodiments, the length of segment 110 of the endovascular device is at least 169.2% of the length of the working segment 120 of the endovascular device. According to some such embodiments, the length of segment 110 of the endovascular device is at least 169.3% of the length of the working segment 120 of the endovascular device. According to some such embodiments, the length of segment 110 of the endovascular device is at least 169.4% of the length of the working segment 120 of the endovascular device. According to some such embodiments, the length of segment 110 of the endovascular device is at least 169.5% of the length of the working segment 120 of the endovascular device. According to some such embodiments, the length of segment 110 of the endovascular device is at least 169.6% of the length of the working segment 120 of the endovascular device. According to some such embodiments, the length of segment 110 of the endovascular device is at least 169.7% of the length of the working segment 120 of the endovascular device. According to some such embodiments, the length of segment 110 of the endovascular device is at least 169.8% of the length of the working segment 120 of the endovascular device. According to some such embodiments, the length of segment 110 of the endovascular device is at least 169.9% of the length of the working segment 120 of the endovascular device. According to some such embodiments, the length of segment 110 of the endovascular device is at least 170% of the length of the working segment 120 of the endovascular device. According to some such embodiments, the length of segment 110 of the endovascular device is at least 170.1% of the length of the working segment 120 of the endovascular device. According to some such embodiments, the length of segment 110 of the endovascular device is at least 170.2% of the length of the working segment 120 of the endovascular device. According to some such embodiments, the length of segment 110 of the endovascular device is at least 170.3% of the length of the working segment 120 of the endovascular device. According to some such embodiments, the length of segment 110 of the endovascular device is at least 170.4% of the length of the working segment 120 of the endovascular device. According to some such embodiments, the length of segment 110 of the endovascular device is at least 170.5% of the length of the working segment 120 of the endovascular device. According to some such embodiments, the length of segment 110 of the endovascular device is at least 170.6% of the length of the working segment 120 of the endovascular device. According to some such embodiments, the length of segment 110 of the endovascular device is at least 170.7% of the length of the working segment 120 of the endovascular device. According to some such embodiments, the length of segment 110 of the endovascular device is at least 170.8% of the length of the working segment 120 of the endovascular device. According to some such embodiments, the length of segment 110 of the endovascular device is at least 170.9% of the length of the working segment 120 of the endovascular device. According to some such embodiments, the length of segment 110 of the endovascular device is at least 171% of the length of the working segment 120 of the endovascular device. According to some such embodiments, the length of segment 110 of the endovascular device is at least 171.1% of the length of the working segment 120 of the endovascular device. According to some such embodiments, the length of segment 110 of the endovascular device is at least 171.2% of the length of the working segment 120 of the endovascular device. According to some such embodiments, the length of segment 110 of the endovascular device is at least 171.3% of the length of the working segment 120 of the endovascular device. According to some such embodiments, the length of segment 110 of the endovascular device is at least 171.4% of the length of the working segment 120 of the endovascular device. According to some such embodiments, the length of segment 110 of the endovascular device is at least 171.5% of the length of the working segment 120 of the endovascular device. According to some such embodiments, the length of segment 110 of the endovascular device is at least 171.6% of the length of the working segment 120 of the endovascular device. According to some such embodiments, the length of segment 110 of the endovascular device is at least 171.7% of the length of the working segment 120 of the endovascular device. According to some such embodiments, the length of segment 110 of the endovascular device is at least 171.8% of the length of the working segment 120 of the endovascular device. According to some such embodiments, the length of segment 110 of the endovascular device is at least 171.9% of the length of the working segment 120 of the endovascular device. According to some such embodiments, the length of segment 110 of the endovascular device is at least 172% of the length of the working segment 120 of the endovascular device. According to some such embodiments, the length of segment 110 of the endovascular device is at least 172.1% of the length of the working segment 120 of the endovascular device. According to some such embodiments, the length of segment 110 of the endovascular device is at least 172.2% of the length of the working segment 120 of the endovascular device. According to some such embodiments, the length of segment 110 of the endovascular device is at least 172.3% of the length of the working segment 120 of the endovascular device. According to some such embodiments, the length of segment 110 of the endovascular device is at least 172.4% of the length of the working segment 120 of the endovascular device. According to some such embodiments, the length of segment 110 of the endovascular device is at least 172.5% of the length of the working segment 120 of the endovascular device. According to some such embodiments, the length of segment 110 of the endovascular device is at least 172.6% of the length of the working segment 120 of the endovascular device. According to some such embodiments, the length of segment 110 of the endovascular device is at least 172.7% of the length of the working segment 120 of the endovascular device. According to some such embodiments, the length of segment 110 of the endovascular device is at least 172.8% of the length of the working segment 120 of the endovascular device. According to some such embodiments, the length of segment 110 of the endovascular device is at least 172.9% of the length of the working segment 120 of the endovascular device. According to some such embodiments, the length of segment 110 of the endovascular device is at least 173% of the length of the working segment 120 of the endovascular device. According to some such embodiments, the length of segment 110 of the endovascular device is at least 173.1% of the length of the working segment 120 of the endovascular device. According to some such embodiments, the length of segment 110 of the endovascular device is at least 173.2% of the length of the working segment 120 of the endovascular device. According to some such embodiments, the length of segment 110 of the endovascular device is at least 173.3% of the length of the working segment 120 of the endovascular device. According to some such embodiments, the length of segment 110 of the endovascular device is at least 173.4% of the length of the working segment 120 of the endovascular device. According to some such embodiments, the length of segment 110 of the endovascular device is at least 173.5% of the length of the working segment 120 of the endovascular device. According to some such embodiments, the length of segment 110 of the endovascular device is at least 173.6% of the length of the working segment 120 of the endovascular device. According to some such embodiments, the length of segment 110 of the endovascular device is at least 173.7% of the length of the working segment 120 of the endovascular device. According to some such embodiments, the length of segment 110 of the endovascular device is at least 173.8% of the length of the working segment 120 of the endovascular device. According to some such embodiments, the length of segment 110 of the endovascular device is at least 173.9% of the length of the working segment 120 of the endovascular device. According to some such embodiments, the length of segment 110 of the endovascular device is at least 174% of the length of the working segment 120 of the endovascular device. According to some such embodiments, the length of segment 110 of the endovascular device is at least 174.1% of the length of the working segment 120 of the endovascular device. According to some such embodiments, the length of segment 110 of the endovascular device is at least 174.2% of the length of the working segment 120 of the endovascular device. According to some such embodiments, the length of segment 110 of the endovascular device is at least 174.3% of the length of the working segment 120 of the endovascular device. According to some such embodiments, the length of segment 110 of the endovascular device is at least 174.4% of the length of the working segment 120 of the endovascular device. According to some such embodiments, the length of segment 110 of the endovascular device is at least 174.5% of the length of the working segment 120 of the endovascular device. According to some such embodiments, the length of segment 110 of the endovascular device is at least 174.6% of the length of the working segment 120 of the endovascular device. According to some such embodiments, the length of segment 110 of the endovascular device is at least 174.7% of the length of the working segment 120 of the endovascular device. According to some such embodiments, the length of segment 110 of the endovascular device is at least 174.8% of the length of the working segment 120 of the endovascular device. According to some such embodiments, the length of segment 110 of the endovascular device is at least 174.9% of the length of the working segment 120 of the endovascular device. According to some such embodiments, the length of segment 110 of the endovascular device is at least 175% of the length of the working segment 120 of the endovascular device.

According to some such embodiments, the length of segment 110 of the endovascular device is at least 175.1% of the length of the working segment 120 of the endovascular device. According to some such embodiments, the length of segment 110 of the endovascular device is at least 175.2% of the length of the working segment 120 of the endovascular device. According to some such embodiments, the length of segment 110 of the endovascular device is at least 175.3% of the length of the working segment 120 of the endovascular device. According to some such embodiments, the length of segment 110 of the endovascular device is at least 175.4% of the length of the working segment 120 of the endovascular device. According to some such embodiments, the length of segment 110 of the endovascular device is at least 175.5% of the length of the working segment 120 of the endovascular device. According to some such embodiments, the length of segment 110 of the endovascular device is at least 175.6% of the length of the working segment 120 of the endovascular device. According to some such embodiments, the length of segment 110 of the endovascular device is at least 175.7% of the length of the working segment 120 of the endovascular device. According to some such embodiments, the length of segment 110 of the endovascular device is at least 175.8% of the length of the working segment 120 of the endovascular device. According to some such embodiments, the length of segment 110 of the endovascular device is at least 175.9% of the length of the working segment 120 of the endovascular device. According to some such embodiments, the length of segment 110 of the endovascular device is at least 176% of the length of the working segment 120 of the endovascular device. According to some such embodiments, the length of segment 110 of the endovascular device is at least 176.1% of the length of the working segment 120 of the endovascular device. According to some such embodiments, the length of segment 110 of the endovascular device is at least 176.2% of the length of the working segment 120 of the endovascular device. According to some such embodiments, the length of segment 110 of the endovascular device is at least 176.3% of the length of the working segment 120 of the endovascular device. According to some such embodiments, the length of segment 110 of the endovascular device is at least 176.4% of the length of the working segment 120 of the endovascular device. According to some such embodiments, the length of segment 110 of the endovascular device is at least 176.5% of the length of the working segment 120 of the endovascular device. According to some such embodiments, the length of segment 110 of the endovascular device is at least 176.6% of the length of the working segment 120 of the endovascular device. According to some such embodiments, the length of segment 110 of the endovascular device is at least 176.7% of the length of the working segment 120 of the endovascular device. According to some such embodiments, the length of segment 110 of the endovascular device is at least 176.8% of the length of the working segment 120 of the endovascular device. According to some such embodiments, the length of segment 110 of the endovascular device is at least 176.9% of the length of the working segment 120 of the endovascular device.

According to some such embodiments, the length of segment 110 of the endovascular device is at least 177.1% of the length of the working segment 120 of the endovascular device. According to some such embodiments, the length of segment 110 of the endovascular device is at least 177.2% of the length of the working segment 120 of the endovascular device. According to some such embodiments, the length of segment 110 of the endovascular device is at least 177.3% of the length of the working segment 120 of the endovascular device. According to some such embodiments, the length of segment 110 of the endovascular device is at least 177.4% of the length of the working segment 120 of the endovascular device. According to some such embodiments, the length of segment 110 of the endovascular device is at least 177.5% of the length of the working segment 120 of the endovascular device. According to some such embodiments, the length of segment 110 of the endovascular device is at least 177.6% of the length of the working segment 120 of the endovascular device. According to some such embodiments, the length of segment 110 of the endovascular device is at least 177.7% of the length of the working segment 120 of the endovascular device. According to some such embodiments, the length of segment 110 of the endovascular device is at least 177.8% of the length of the working segment 120 of the endovascular device. According to some such embodiments, the length of segment 110 of the endovascular device is at least 177.9% of the length of the working segment 120 of the endovascular device. According to some such embodiments, the length of segment 110 of the endovascular device is at least 178% of the length of the working segment 120 of the endovascular device. According to some such embodiments, the length of segment 110 of the endovascular device is at least 178.1% of the length of the working segment 120 of the endovascular device. According to some such embodiments, the length of segment 110 of the endovascular device is at least 178.2% of the length of the working segment 120 of the endovascular device. According to some such embodiments, the length of segment 110 of the endovascular device is at least 178.3% of the length of the working segment 120 of the endovascular device. According to some such embodiments, the length of segment 110 of the endovascular device is at least 178.4% of the length of the working segment 120 of the endovascular device. According to some such embodiments, the length of segment 110 of the endovascular device is at least 178.5% of the length of the working segment 120 of the endovascular device. According to some such embodiments, the length of segment 110 of the endovascular device is at least 178.6% of the length of the working segment 120 of the endovascular device. According to some such embodiments, the length of segment 110 of the endovascular device is at least 178.7% of the length of the working segment 120 of the endovascular device. According to some such embodiments, the length of segment 110 of the endovascular device is at least 178.8% of the length of the working segment 120 of the endovascular device. According to some such embodiments, the length of segment 110 of the endovascular device is at least 178.9% of the length of the working segment 120 of the endovascular device. According to some such embodiments, the length of segment 110 of the endovascular device is at least 179% of the length of the working segment 120 of the endovascular device. According to some such embodiments, the length of segment 110 of the endovascular device is at least 179.1% of the length of the working segment 120 of the endovascular device. According to some such embodiments, the length of segment 110 of the endovascular device is at least 179.2% of the length of the working segment 120 of the endovascular device. According to some such embodiments, the length of segment 110 of the endovascular device is at least 179.3% of the length of the working segment 120 of the endovascular device. According to some such embodiments, the length of segment 110 of the endovascular device is at least 179.4% of the length of the working segment 120 of the endovascular device. According to some such embodiments, the length of segment 110 of the endovascular device is at least 179.5% of the length of the working segment 120 of the endovascular device. According to some such embodiments, the length of segment 110 of the endovascular device is at least 179.6% of the length of the working segment 120 of the endovascular device. According to some such embodiments, the length of segment 110 of the endovascular device is at least 179.7% of the length of the working segment 120 of the endovascular device. According to some such embodiments, the length of segment 110 of the endovascular device is at least 179.8% of the length of the working segment 120 of the endovascular device. According to some such embodiments, the length of segment 110 of the endovascular device is at least 179.9% of the length of the working segment 120 of the endovascular device. According to some such embodiments, the length of segment 110 of the endovascular device is at least 180% of the length of the working segment 120 of the endovascular device. According to some such embodiments, the length of segment 110 of the endovascular device is at least 180.1% of the length of the working segment 120 of the endovascular device. According to some such embodiments, the length of segment 110 of the endovascular device is at least 180.2% of the length of the working segment 120 of the endovascular device. According to some such embodiments, the length of segment 110 of the endovascular device is at least 180.3% of the length of the working segment 120 of the endovascular device. According to some such embodiments, the length of segment 110 of the endovascular device is at least 180.4% of the length of the working segment 120 of the endovascular device. According to some such embodiments, the length of segment 110 of the endovascular device is at least 180.5% of the length of the working segment 120 of the endovascular device. According to some such embodiments, the length of segment 110 of the endovascular device is at least 180.6% of the length of the working segment 120 of the endovascular device. According to some such embodiments, the length of segment 110 of the endovascular device is at least 180.7% of the length of the working segment 120 of the endovascular device. According to some such embodiments, the length of segment 110 of the endovascular device is at least 180.8% of the length of the working segment 120 of the endovascular device. According to some such embodiments, the length of segment 110 of the endovascular device is at least 180.9% of the length of the working segment 120 of the endovascular device. According to some such embodiments, the length of segment 110 of the endovascular device is at least 181% of the length of the working segment 120 of the endovascular device. According to some such embodiments, the length of segment 110 of the endovascular device is at least 181.1% of the length of the working segment 120 of the endovascular device. According to some such embodiments, the length of segment 110 of the endovascular device is at least 181.2% of the length of the working segment 120 of the endovascular device. According to some such embodiments, the length of segment 110 of the endovascular device is at least 181.3% of the length of the working segment 120 of the endovascular device. According to some such embodiments, the length of segment 110 of the endovascular device is at least 181.4% of the length of the working segment 120 of the endovascular device. According to some such embodiments, the length of segment 110 of the endovascular device is at least 181.5% of the length of the working segment 120 of the endovascular device. According to some such embodiments, the length of segment 110 of the endovascular device is at least 181.6% of the length of the working segment 120 of the endovascular device. According to some such embodiments, the length of segment 110 of the endovascular device is at least 181.7% of the length of the working segment 120 of the endovascular device. According to some such embodiments, the length of segment 110 of the endovascular device is at least 181.8% of the length of the working segment 120 of the endovascular device. According to some such embodiments, the length of segment 110 of the endovascular device is at least 181.9% of the length of the working segment 120 of the endovascular device. According to some such embodiments, the length of segment 110 of the endovascular device is at least 182% of the length of the working segment 120 of the endovascular device. According to some such embodiments, the length of segment 110 of the endovascular device is at least 182.1% of the length of the working segment 120 of the endovascular device. According to some such embodiments, the length of segment 110 of the endovascular device is at least 182.2% of the length of the working segment 120 of the endovascular device. According to some such embodiments, the length of segment 110 of the endovascular device is at least 182.3% of the length of the working segment 120 of the endovascular device. According to some such embodiments, the length of segment 110 of the endovascular device is at least 182.4% of the length of the working segment 120 of the endovascular device. According to some such embodiments, the length of segment 110 of the endovascular device is at least 182.5% of the length of the working segment 120 of the endovascular device. According to some such embodiments, the length of segment 110 of the endovascular device is at least 182.6% of the length of the working segment 120 of the endovascular device. According to some such embodiments, the length of segment 110 of the endovascular device is at least 182.7% of the length of the working segment 120 of the endovascular device. According to some such embodiments, the length of segment 110 of the endovascular device is at least 182.8% of the length of the working segment 120 of the endovascular device. According to some such embodiments, the length of segment 110 of the endovascular device is at least 182.9% of the length of the working segment 120 of the endovascular device. According to some such embodiments, the length of segment 110 of the endovascular device is at least 183% of the length of the working segment 120 of the endovascular device. According to some such embodiments, the length of segment 110 of the endovascular device is at least 183.1% of the length of the working segment 120 of the endovascular device. According to some such embodiments, the length of segment 110 of the endovascular device is at least 183.2% of the length of the working segment 120 of the endovascular device. According to some such embodiments, the length of segment 110 of the endovascular device is at least 183.3% of the length of the working segment 120 of the endovascular device. According to some such embodiments, the length of segment 110 of the endovascular device is at least 183.4% of the length of the working segment 120 of the endovascular device. According to some such embodiments, the length of segment 110 of the endovascular device is at least 183.5% of the length of the working segment 120 of the endovascular device. According to some such embodiments, the length of segment 110 of the endovascular device is at least 183.6% of the length of the working segment 120 of the endovascular device. According to some such embodiments, the length of segment 110 of the endovascular device is at least 183.7% of the length of the working segment 120 of the endovascular device. According to some such embodiments, the length of segment 110 of the endovascular device is at least 183.8% of the length of the working segment 120 of the endovascular device. According to some such embodiments, the length of segment 110 of the endovascular device is at least 183.9% of the length of the working segment 120 of the endovascular device. According to some such embodiments, the length of segment 110 of the endovascular device is at least 184% of the length of the working segment 120 of the endovascular device. According to some such embodiments, the length of segment 110 of the endovascular device is at least 184.1% of the length of the working segment 120 of the endovascular device. According to some such embodiments, the length of segment 110 of the endovascular device is at least 184.2% of the length of the working segment 120 of the endovascular device. According to some such embodiments, the length of segment 110 of the endovascular device is at least 184.3% of the length of the working segment 120 of the endovascular device. According to some such embodiments, the length of segment 110 of the endovascular device is at least 184.4% of the length of the working segment 120 of the endovascular device. According to some such embodiments, the length of segment 110 of the endovascular device is at least 184.5% of the length of the working segment 120 of the endovascular device. According to some such embodiments, the length of segment 110 of the endovascular device is at least 184.6% of the length of the working segment 120 of the endovascular device. According to some such embodiments, the length of segment 110 of the endovascular device is at least 184.7% of the length of the working segment 120 of the endovascular device. According to some such embodiments, the length of segment 110 of the endovascular device is at least 184.8% of the length of the working segment 120 of the endovascular device. According to some such embodiments, the length of segment 110 of the endovascular device is at least 184.9% of the length of the working segment 120 of the endovascular device. According to some such embodiments, the length of segment 110 of the endovascular device is at least 185% of the length of the working segment 120 of the endovascular device. According to some such embodiments, the length of segment 110 of the endovascular device is at least 185.1% of the length of the working segment 120 of the endovascular device. According to some such embodiments, the length of segment 110 of the endovascular device is at least 185.2% of the length of the working segment 120 of the endovascular device. According to some such embodiments, the length of segment 110 of the endovascular device is at least 185.3% of the length of the working segment 120 of the endovascular device. According to some such embodiments, the length of segment 110 of the endovascular device is at least 185.4% of the length of the working segment 120 of the endovascular device. According to some such embodiments, the length of segment 110 of the endovascular device is at least 185.5% of the length of the working segment 120 of the endovascular device. According to some such embodiments, the length of segment 110 of the endovascular device is at least 185.6% of the length of the working segment 120 of the endovascular device. According to some such embodiments, the length of segment 110 of the endovascular device is at least 185.7% of the length of the working segment 120 of the endovascular device. According to some such embodiments, the length of segment 110 of the endovascular device is at least 185.8% of the length of the working segment 120 of the endovascular device. According to some such embodiments, the length of segment 110 of the endovascular device is at least 185.9% of the length of the working segment 120 of the endovascular device. According to some such embodiments, the length of segment 110 of the endovascular device is at least 186% of the length of the working segment 120 of the endovascular device. According to some such embodiments, the length of segment 110 of the endovascular device is at least 186.1% of the length of the working segment 120 of the endovascular device. According to some such embodiments, the length of segment 110 of the endovascular device is at least 186.2% of the length of the working segment 120 of the endovascular device. According to some such embodiments, the length of segment 110 of the endovascular device is at least 186.3% of the length of the working segment 120 of the endovascular device. According to some such embodiments, the length of segment 110 of the endovascular device is at least 186.4% of the length of the working segment 120 of the endovascular device. According to some such embodiments, the length of segment 110 of the endovascular device is at least 186.5% of the length of the working segment 120 of the endovascular device. According to some such embodiments, the length of segment 110 of the endovascular device is at least 186.6% of the length of the working segment 120 of the endovascular device. According to some such embodiments, the length of segment 110 of the endovascular device is at least 186.7% of the length of the working segment 120 of the endovascular device. According to some such embodiments, the length of segment 110 of the endovascular device is at least 186.8% of the length of the working segment 120 of the endovascular device. According to some such embodiments, the length of segment 110 of the endovascular device is at least 186.9% of the length of the working segment 120 of the endovascular device. According to some such embodiments, the length of segment 110 of the endovascular device is at least 187% of the length of the working segment 120 of the endovascular device. According to some such embodiments, the length of segment 110 of the endovascular device is at least 187.1% of the length of the working segment 120 of the endovascular device. According to some such embodiments, the length of segment 110 of the endovascular device is at least 187.2% of the length of the working segment 120 of the endovascular device. According to some such embodiments, the length of segment 110 of the endovascular device is at least 187.3% of the length of the working segment 120 of the endovascular device. According to some such embodiments, the length of segment 110 of the endovascular device is at least 187.4% of the length of the working segment 120 of the endovascular device. According to some such embodiments, the length of segment 110 of the endovascular device is at least 187.5% of the length of the working segment 120 of the endovascular device. According to some such embodiments, the length of segment 110 of the endovascular device is at least 187.6% of the length of the working segment 120 of the endovascular device. According to some such embodiments, the length of segment 110 of the endovascular device is at least 187.7% of the length of the working segment 120 of the endovascular device. According to some such embodiments, the length of segment 110 of the endovascular device is at least 187.8% of the length of the working segment 120 of the endovascular device. According to some such embodiments, the length of segment 110 of the endovascular device is at least 187.9% of the length of the working segment 120 of the endovascular device. According to some such embodiments, the length of segment 110 of the endovascular device is at least 188% of the length of the working segment 120 of the endovascular device. According to some such embodiments, the length of segment 110 of the endovascular device is at least 188.1% of the length of the working segment 120 of the endovascular device. According to some such embodiments, the length of segment 110 of the endovascular device is at least 188.2% of the length of the working segment 120 of the endovascular device. According to some such embodiments, the length of segment 110 of the endovascular device is at least 188.3% of the length of the working segment 120 of the endovascular device. According to some such embodiments, the length of segment 110 of the endovascular device is at least 188.4% of the length of the working segment 120 of the endovascular device. According to some such embodiments, the length of segment 110 of the endovascular device is at least 188.5% of the length of the working segment 120 of the endovascular device. According to some such embodiments, the length of segment 110 of the endovascular device is at least 188.6% of the length of the working segment 120 of the endovascular device. According to some such embodiments, the length of segment 110 of the endovascular device is at least 188.7% of the length of the working segment 120 of the endovascular device. According to some such embodiments, the length of segment 110 of the endovascular device is at least 188.8% of the length of the working segment 120 of the endovascular device. According to some such embodiments, the length of segment 110 of the endovascular device is at least 188.9% of the length of the working segment 120 of the endovascular device. According to some such embodiments, the length of segment 110 of the endovascular device is at least 189% of the length of the working segment 120 of the endovascular device. According to some such embodiments, the length of segment 110 of the endovascular device is at least 189.1% of the length of the working segment 120 of the endovascular device. According to some such embodiments, the length of segment 110 of the endovascular device is at least 189.2% of the length of the working segment 120 of the endovascular device. According to some such embodiments, the length of segment 110 of the endovascular device is at least 189.3% of the length of the working segment 120 of the endovascular device. According to some such embodiments, the length of segment 110 of the endovascular device is at least 189.4% of the length of the working segment 120 of the endovascular device. According to some such embodiments, the length of segment 110 of the endovascular device is at least 189.5% of the length of the working segment 120 of the endovascular device. According to some such embodiments, the length of segment 110 of the endovascular device is at least 189.6% of the length of the working segment 120 of the endovascular device. According to some such embodiments, the length of segment 110 of the endovascular device is at least 189.7% of the length of the working segment 120 of the endovascular device. According to some such embodiments, the length of segment 110 of the endovascular device is at least 189.8% of the length of the working segment 120 of the endovascular device. According to some such embodiments, the length of segment 110 of the endovascular device is at least 189.9% of the length of the working segment 120 of the endovascular device.

According to some such embodiments, the length of segment 110 of the endovascular device is at least 190% of the length of the working segment 120 of the endovascular device. According to some such embodiments, the length of segment 110 of the endovascular device is at least 190.1% of the length of the working segment 120 of the endovascular device. According to some such embodiments, the length of segment 110 of the endovascular device is at least 190.2% of the length of the working segment 120 of the endovascular device. According to some such embodiments, the length of segment 110 of the endovascular device is at least 190.3% of the length of the working segment 120 of the endovascular device. According to some such embodiments, the length of segment 110 of the endovascular device is at least 190.4% of the length of the working segment 120 of the endovascular device. According to some such embodiments, the length of segment 110 of the endovascular device is at least 190.5% of the length of the working segment 120 of the endovascular device. According to some such embodiments, the length of segment 110 of the endovascular device is at least 190.6% of the length of the working segment 120 of the endovascular device. According to some such embodiments, the length of segment 110 of the endovascular device is at least 190.7% of the length of the working segment 120 of the endovascular device. According to some such embodiments, the length of segment 110 of the endovascular device is at least 190.8% of the length of the working segment 120 of the endovascular device. According to some such embodiments, the length of segment 110 of the endovascular device is at least 190.9% of the length of the working segment 120 of the endovascular device. According to some such embodiments, the length of segment 110 of the endovascular device is at least 191% of the length of the working segment 120 of the endovascular device. According to some such embodiments, the length of segment 110 of the endovascular device is at least 191.1% of the length of the working segment 120 of the endovascular device. According to some such embodiments, the length of segment 110 of the endovascular device is at least 191.2% of the length of the working segment 120 of the endovascular device. According to some such embodiments, the length of segment 110 of the endovascular device is at least 191.3% of the length of the working segment 120 of the endovascular device. According to some such embodiments, the length of segment 110 of the endovascular device is at least 191.4% of the length of the working segment 120 of the endovascular device. According to some such embodiments, the length of segment 110 of the endovascular device is at least 191.5% of the length of the working segment 120 of the endovascular device. According to some such embodiments, the length of segment 110 of the endovascular device is at least 191.6% of the length of the working segment 120 of the endovascular device. According to some such embodiments, the length of segment 110 of the endovascular device is at least 191.7% of the length of the working segment 120 of the endovascular device. According to some such embodiments, the length of segment 110 of the endovascular device is at least 191.8% of the length of the working segment 120 of the endovascular device. According to some such embodiments, the length of segment 110 of the endovascular device is at least 191.9% of the length of the working segment 120 of the endovascular device. According to some such embodiments, the length of segment 110 of the endovascular device is at least 192% of the length of the working segment 120 of the endovascular device. According to some such embodiments, the length of segment 110 of the endovascular device is at least 192.1% of the length of the working segment 120 of the endovascular device. According to some such embodiments, the length of segment 110 of the endovascular device is at least 192.2% of the length of the working segment 120 of the endovascular device. According to some such embodiments, the length of segment 110 of the endovascular device is at least 192.3% of the length of the working segment 120 of the endovascular device. According to some such embodiments, the length of segment 110 of the endovascular device is at least 192.4% of the length of the working segment 120 of the endovascular device. According to some such embodiments, the length of segment 110 of the endovascular device is at least 192.5% of the length of the working segment 120 of the endovascular device. According to some such embodiments, the length of segment 110 of the endovascular device is at least 192.6% of the length of the working segment 120 of the endovascular device. According to some such embodiments, the length of segment 110 of the endovascular device is at least 192.7% of the length of the working segment 120 of the endovascular device. According to some such embodiments, the length of segment 110 of the endovascular device is at least 192.8% of the length of the working segment 120 of the endovascular device. According to some such embodiments, the length of segment 110 of the endovascular device is at least 192.9% of the length of the working segment 120 of the endovascular device. According to some such embodiments, the length of segment 110 of the endovascular device is at least 193% of the length of the working segment 120 of the endovascular device. According to some such embodiments, the length of segment 110 of the endovascular device is at least 193.1% of the length of the working segment 120 of the endovascular device. According to some such embodiments, the length of segment 110 of the endovascular device is at least 193.2% of the length of the working segment 120 of the endovascular device. According to some such embodiments, the length of segment 110 of the endovascular device is at least 193.3% of the length of the working segment 120 of the endovascular device. According to some such embodiments, the length of segment 110 of the endovascular device is at least 193.4% of the length of the working segment 120 of the endovascular device. According to some such embodiments, the length of segment 110 of the endovascular device is at least 193.5% of the length of the working segment 120 of the endovascular device. According to some such embodiments, the length of segment 110 of the endovascular device is at least 193.6% of the length of the working segment 120 of the endovascular device. According to some such embodiments, the length of segment 110 of the endovascular device is at least 193.7% of the length of the working segment 120 of the endovascular device. According to some such embodiments, the length of segment 110 of the endovascular device is at least 193.8% of the length of the working segment 120 of the endovascular device. According to some such embodiments, the length of segment 110 of the endovascular device is at least 193.9% of the length of the working segment 120 of the endovascular device. According to some such embodiments, the length of segment 110 of the endovascular device is at least 194% of the length of the working segment 120 of the endovascular device. According to some such embodiments, the length of segment 110 of the endovascular device is at least 194.1% of the length of the working segment 120 of the endovascular device. According to some such embodiments, the length of segment 110 of the endovascular device is at least 194.2% of the length of the working segment 120 of the endovascular device. According to some such embodiments, the length of segment 110 of the endovascular device is at least 194.3% of the length of the working segment 120 of the endovascular device. According to some such embodiments, the length of segment 110 of the endovascular device is at least 194.4% of the length of the working segment 120 of the endovascular device. According to some such embodiments, the length of segment 110 of the endovascular device is at least 194.5% of the length of the working segment 120 of the endovascular device. According to some such embodiments, the length of segment 110 of the endovascular device is at least 194.6% of the length of the working segment 120 of the endovascular device. According to some such embodiments, the length of segment 110 of the endovascular device is at least 194.7% of the length of the working segment 120 of the endovascular device. According to some such embodiments, the length of segment 110 of the endovascular device is at least 194.8% of the length of the working segment 120 of the endovascular device. According to some such embodiments, the length of segment 110 of the endovascular device is at least 194.9% of the length of the working segment 120 of the endovascular device. According to some such embodiments, the length of segment 110 of the endovascular device is at least 195% of the length of the working segment 120 of the endovascular device. According to some such embodiments, the length of segment 110 of the endovascular device is at least 195.1% of the length of the working segment 120 of the endovascular device. According to some such embodiments, the length of segment 110 of the endovascular device is at least 195.2% of the length of the working segment 120 of the endovascular device. According to some such embodiments, the length of segment 110 of the endovascular device is at least 195.3% of the length of the working segment 120 of the endovascular device. According to some such embodiments, the length of segment 110 of the endovascular device is at least 195.4% of the length of the working segment 120 of the endovascular device. According to some such embodiments, the length of segment 110 of the endovascular device is at least 195.5% of the length of the working segment 120 of the endovascular device. According to some such embodiments, the length of segment 110 of the endovascular device is at least 195.6% of the length of the working segment 120 of the endovascular device. According to some such embodiments, the length of segment 110 of the endovascular device is at least 195.7% of the length of the working segment 120 of the endovascular device. According to some such embodiments, the length of segment 110 of the endovascular device is at least 195.8% of the length of the working segment 120 of the endovascular device. According to some such embodiments, the length of segment 110 of the endovascular device is at least 195.9% of the length of the working segment 120 of the endovascular device. According to some such embodiments, the length of segment 110 of the endovascular device is at least 196% of the length of the working segment 120 of the endovascular device. According to some such embodiments, the length of segment 110 of the endovascular device is at least 196.1% of the length of the working segment 120 of the endovascular device. According to some such embodiments, the length of segment 110 of the endovascular device is at least 196.2% of the length of the working segment 120 of the endovascular device. According to some such embodiments, the length of segment 110 of the endovascular device is at least 196.3% of the length of the working segment 120 of the endovascular device. According to some such embodiments, the length of segment 110 of the endovascular device is at least 196.4% of the length of the working segment 120 of the endovascular device. According to some such embodiments, the length of segment 110 of the endovascular device is at least 196.5% of the length of the working segment 120 of the endovascular device. According to some such embodiments, the length of segment 110 of the endovascular device is at least 196.6% of the length of the working segment 120 of the endovascular device. According to some such embodiments, the length of segment 110 of the endovascular device is at least 196.7% of the length of the working segment 120 of the endovascular device. According to some such embodiments, the length of segment 110 of the endovascular device is at least 196.8% of the length of the working segment 120 of the endovascular device. According to some such embodiments, the length of segment 110 of the endovascular device is at least 196.9% of the length of the working segment 120 of the endovascular device. According to some such embodiments, the length of segment 110 of the endovascular device is at least 197% of the length of the working segment 120 of the endovascular device. According to some such embodiments, the length of segment 110 of the endovascular device is at least 197.1% of the length of the working segment 120 of the endovascular device. According to some such embodiments, the length of segment 110 of the endovascular device is at least 197.2% of the length of the working segment 120 of the endovascular device. According to some such embodiments, the length of segment 110 of the endovascular device is at least 197.3% of the length of the working segment 120 of the endovascular device. According to some such embodiments, the length of segment 110 of the endovascular device is at least 197.4% of the length of the working segment 120 of the endovascular device. According to some such embodiments, the length of segment 110 of the endovascular device is at least 197.5% of the length of the working segment 120 of the endovascular device. According to some such embodiments, the length of segment 110 of the endovascular device is at least 197.6% of the length of the working segment 120 of the endovascular device. According to some such embodiments, the length of segment 110 of the endovascular device is at least 197.7% of the length of the working segment 120 of the endovascular device. According to some such embodiments, the length of segment 110 of the endovascular device is at least 197.8% of the length of the working segment 120 of the endovascular device. According to some such embodiments, the length of segment 110 of the endovascular device is at least 197.9% of the length of the working segment 120 of the endovascular device. According to some such embodiments, the length of segment 110 of the endovascular device is at least 198% of the length of the working segment 120 of the endovascular device. According to some such embodiments, the length of segment 110 of the endovascular device is at least 198.1% of the length of the working segment 120 of the endovascular device. According to some such embodiments, the length of segment 110 of the endovascular device is at least 198.2% of the length of the working segment 120 of the endovascular device. According to some such embodiments, the length of segment 110 of the endovascular device is at least 198.3% of the length of the working segment 120 of the endovascular device. According to some such embodiments, the length of segment 110 of the endovascular device is at least 198.4% of the length of the working segment 120 of the endovascular device. According to some such embodiments, the length of segment 110 of the endovascular device is at least 198.5% of the length of the working segment 120 of the endovascular device. According to some such embodiments, the length of segment 110 of the endovascular device is at least 198.6% of the length of the working segment 120 of the endovascular device. According to some such embodiments, the length of segment 110 of the endovascular device is at least 198.7% of the length of the working segment 120 of the endovascular device. According to some such embodiments, the length of segment 110 of the endovascular device is at least 198.8% of the length of the working segment 120 of the endovascular device. According to some such embodiments, the length of segment 110 of the endovascular device is at least 198.9% of the length of the working segment 120 of the endovascular device. According to some such embodiments, the length of segment 110 of the endovascular device is at least 199% of the length of the working segment 120 of the endovascular device. According to some such embodiments, the length of segment 110 of the endovascular device is at least 199.1% of the length of the working segment 120 of the endovascular device. According to some such embodiments, the length of segment 110 of the endovascular device is at least 199.2% of the length of the working segment 120 of the endovascular device. According to some such embodiments, the length of segment 110 of the endovascular device is at least 199.3% of the length of the working segment 120 of the endovascular device. According to some such embodiments, the length of segment 110 of the endovascular device is at least 199.4% of the length of the working segment 120 of the endovascular device. According to some such embodiments, the length of segment 110 of the endovascular device is at least 199.5% of the length of the working segment 120 of the endovascular device. According to some such embodiments, the length of segment 110 of the endovascular device is at least 199.6% of the length of the working segment 120 of the endovascular device. According to some such embodiments, the length of segment 110 of the endovascular device is at least 199.7% of the length of the working segment 120 of the endovascular device. According to some such embodiments, the length of segment 110 of the endovascular device is at least 199.8% of the length of the working segment 120 of the endovascular device. According to some such embodiments, the length of segment 110 of the endovascular device is at least 199.9% of the length of the working segment 120 of the endovascular device.

According to some such embodiments, the length of segment 110 of the endovascular device is at least 200% of the length of the working segment 120 of the endovascular device. According to some such embodiments, the length of segment 110 of the endovascular device is at least 200.1% of the length of the working segment 120 of the endovascular device. According to some such embodiments, the length of segment 110 of the endovascular device is at least 200.2% of the length of the working segment 120 of the endovascular device. According to some such embodiments, the length of segment 110 of the endovascular device is at least 200.3% of the length of the working segment 120 of the endovascular device. According to some such embodiments, the length of segment 110 of the endovascular device is at least 200.4% of the length of the working segment 120 of the endovascular device. According to some such embodiments, the length of segment 110 of the endovascular device is at least 200.5% of the length of the working segment 120 of the endovascular device. According to some such embodiments, the length of segment 110 of the endovascular device is at least 200.6% of the length of the working segment 120 of the endovascular device. According to some such embodiments, the length of segment 110 of the endovascular device is at least 200.7% of the length of the working segment 120 of the endovascular device. According to some such embodiments, the length of segment 110 of the endovascular device is at least 200.8% of the length of the working segment 120 of the endovascular device. According to some such embodiments, the length of segment 110 of the endovascular device is at least 200.9% of the length of the working segment 120 of the endovascular device. According to some such embodiments, the length of segment 110 of the endovascular device is at least 201% of the length of the working segment 120 of the endovascular device. According to some such embodiments, the length of segment 110 of the endovascular device is at least 201.1% of the length of the working segment 120 of the endovascular device. According to some such embodiments, the length of segment 110 of the endovascular device is at least 201.2% of the length of the working segment 120 of the endovascular device. According to some such embodiments, the length of segment 110 of the endovascular device is at least 201.3% of the length of the working segment 120 of the endovascular device. According to some such embodiments, the length of segment 110 of the endovascular device is at least 201.4% of the length of the working segment 120 of the endovascular device. According to some such embodiments, the length of segment 110 of the endovascular device is at least 201.5% of the length of the working segment 120 of the endovascular device. According to some such embodiments, the length of segment 110 of the endovascular device is at least 201.6% of the length of the working segment 120 of the endovascular device. According to some such embodiments, the length of segment 110 of the endovascular device is at least 201.7% of the length of the working segment 120 of the endovascular device. According to some such embodiments, the length of segment 110 of the endovascular device is at least 201.8% of the length of the working segment 120 of the endovascular device. According to some such embodiments, the length of segment 110 of the endovascular device is at least 201.9% of the length of the working segment 120 of the endovascular device. According to some such embodiments, the length of segment 110 of the endovascular device is at least 202% of the length of the working segment 120 of the endovascular device. According to some such embodiments, the length of segment 110 of the endovascular device is at least 202.1% of the length of the working segment 120 of the endovascular device. According to some such embodiments, the length of segment 110 of the endovascular device is at least 202.2% of the length of the working segment 120 of the endovascular device. According to some such embodiments, the length of segment 110 of the endovascular device is at least 202.3% of the length of the working segment 120 of the endovascular device. According to some such embodiments, the length of segment 110 of the endovascular device is at least 202.4% of the length of the working segment 120 of the endovascular device. According to some such embodiments, the length of segment 110 of the endovascular device is at least 202.5% of the length of the working segment 120 of the endovascular device. According to some such embodiments, the length of segment 110 of the endovascular device is at least 202.6% of the length of the working segment 120 of the endovascular device. According to some such embodiments, the length of segment 110 of the endovascular device is at least 202.7% of the length of the working segment 120 of the endovascular device. According to some such embodiments, the length of segment 110 of the endovascular device is at least 202.8% of the length of the working segment 120 of the endovascular device. According to some such embodiments, the length of segment 110 of the endovascular device is at least 202.9% of the length of the working segment 120 of the endovascular device. According to some such embodiments, the length of segment 110 of the endovascular device is at least 203% of the length of the working segment 120 of the endovascular device. According to some such embodiments, the length of segment 110 of the endovascular device is at least 203.1% of the length of the working segment 120 of the endovascular device. According to some such embodiments, the length of segment 110 of the endovascular device is at least 203.2% of the length of the working segment 120 of the endovascular device. According to some such embodiments, the length of segment 110 of the endovascular device is at least 203.3% of the length of the working segment 120 of the endovascular device. According to some such embodiments, the length of segment 110 of the endovascular device is at least 203.4% of the length of the working segment 120 of the endovascular device. According to some such embodiments, the length of segment 110 of the endovascular device is at least 203.5% of the length of the working segment 120 of the endovascular device. According to some such embodiments, the length of segment 110 of the endovascular device is at least 203.6% of the length of the working segment 120 of the endovascular device. According to some such embodiments, the length of segment 110 of the endovascular device is at least 203.7% of the length of the working segment 120 of the endovascular device. According to some such embodiments, the length of segment 110 of the endovascular device is at least 203.8% of the length of the working segment 120 of the endovascular device. According to some such embodiments, the length of segment 110 of the endovascular device is at least 203.9% of the length of the working segment 120 of the endovascular device. According to some such embodiments, the length of segment 110 of the endovascular device is at least 204% of the length of the working segment 120 of the endovascular device. According to some such embodiments, the length of segment 110 of the endovascular device is at least 204.1% of the length of the working segment 120 of the endovascular device. According to some such embodiments, the length of segment 110 of the endovascular device is at least 204.2% of the length of the working segment 120 of the endovascular device. According to some such embodiments, the length of segment 110 of the endovascular device is at least 204.3% of the length of the working segment 120 of the endovascular device. According to some such embodiments, the length of segment 110 of the endovascular device is at least 204.4% of the length of the working segment 120 of the endovascular device. According to some such embodiments, the length of segment 110 of the endovascular device is at least 204.5% of the length of the working segment 120 of the endovascular device. According to some such embodiments, the length of segment 110 of the endovascular device is at least 204.6% of the length of the working segment 120 of the endovascular device. According to some such embodiments, the length of segment 110 of the endovascular device is at least 204.7% of the length of the working segment 120 of the endovascular device. According to some such embodiments, the length of segment 110 of the endovascular device is at least 204.8% of the length of the working segment 120 of the endovascular device. According to some such embodiments, the length of segment 110 of the endovascular device is at least 204.9% of the length of the working segment 120 of the endovascular device. According to some such embodiments, the length of segment 110 of the endovascular device is at least 205% of the length of the working segment 120 of the endovascular device. According to some such embodiments, the length of segment 110 of the endovascular device is at least 205.1% of the length of the working segment 120 of the endovascular device. According to some such embodiments, the length of segment 110 of the endovascular device is at least 205.2% of the length of the working segment 120 of the endovascular device. According to some such embodiments, the length of segment 110 of the endovascular device is at least 205.3% of the length of the working segment 120 of the endovascular device. According to some such embodiments, the length of segment 110 of the endovascular device is at least 205.4% of the length of the working segment 120 of the endovascular device. According to some such embodiments, the length of segment 110 of the endovascular device is at least 205.5% of the length of the working segment 120 of the endovascular device. According to some such embodiments, the length of segment 110 of the endovascular device is at least 205.6% of the length of the working segment 120 of the endovascular device. According to some such embodiments, the length of segment 110 of the endovascular device is at least 205.7% of the length of the working segment 120 of the endovascular device. According to some such embodiments, the length of segment 110 of the endovascular device is at least 205.8% of the length of the working segment 120 of the endovascular device. According to some such embodiments, the length of segment 110 of the endovascular device is at least 205.9% of the length of the working segment 120 of the endovascular device. According to some such embodiments, the length of segment 110 of the endovascular device is at least 206% of the length of the working segment 120 of the endovascular device. According to some such embodiments, the length of segment 110 of the endovascular device is at least 206.1% of the length of the working segment 120 of the endovascular device. According to some such embodiments, the length of segment 110 of the endovascular device is at least 206.2% of the length of the working segment 120 of the endovascular device. According to some such embodiments, the length of segment 110 of the endovascular device is at least 206.3% of the length of the working segment 120 of the endovascular device. According to some such embodiments, the length of segment 110 of the endovascular device is at least 206.4% of the length of the working segment 120 of the endovascular device. According to some such embodiments, the length of segment 110 of the endovascular device is at least 206.5% of the length of the working segment 120 of the endovascular device. According to some such embodiments, the length of segment 110 of the endovascular device is at least 206.6% of the length of the working segment 120 of the endovascular device. According to some such embodiments, the length of segment 110 of the endovascular device is at least 206.7% of the length of the working segment 120 of the endovascular device. According to some such embodiments, the length of segment 110 of the endovascular device is at least 206.8% of the length of the working segment 120 of the endovascular device. According to some such embodiments, the length of segment 110 of the endovascular device is at least 206.9% of the length of the working segment 120 of the endovascular device. According to some such embodiments, the length of segment 110 of the endovascular device is at least 207% of the length of the working segment 120 of the endovascular device. According to some such embodiments, the length of segment 110 of the endovascular device is at least 207.1% of the length of the working segment 120 of the endovascular device. According to some such embodiments, the length of segment 110 of the endovascular device is at least 207.2% of the length of the working segment 120 of the endovascular device. According to some such embodiments, the length of segment 110 of the endovascular device is at least 207.3% of the length of the working segment 120 of the endovascular device. According to some such embodiments, the length of segment 110 of the endovascular device is at least 207.4% of the length of the working segment 120 of the endovascular device. According to some such embodiments, the length of segment 110 of the endovascular device is at least 207.5% of the length of the working segment 120 of the endovascular device. According to some such embodiments, the length of segment 110 of the endovascular device is at least 207.6% of the length of the working segment 120 of the endovascular device. According to some such embodiments, the length of segment 110 of the endovascular device is at least 207.7% of the length of the working segment 120 of the endovascular device. According to some such embodiments, the length of segment 110 of the endovascular device is at least 207.8% of the length of the working segment 120 of the endovascular device. According to some such embodiments, the length of segment 110 of the endovascular device is at least 207.9% of the length of the working segment 120 of the endovascular device. According to some such embodiments, the length of segment 110 of the endovascular device is at least 208% of the length of the working segment 120 of the endovascular device. According to some such embodiments, the length of segment 110 of the endovascular device is at least 208.1% of the length of the working segment 120 of the endovascular device. According to some such embodiments, the length of segment 110 of the endovascular device is at least 208.2% of the length of the working segment 120 of the endovascular device. According to some such embodiments, the length of segment 110 of the endovascular device is at least 208.3% of the length of the working segment 120 of the endovascular device. According to some such embodiments, the length of segment 110 of the endovascular device is at least 208.4% of the length of the working segment 120 of the endovascular device. According to some such embodiments, the length of segment 110 of the endovascular device is at least 208.5% of the length of the working segment 120 of the endovascular device. According to some such embodiments, the length of segment 110 of the endovascular device is at least 208.6% of the length of the working segment 120 of the endovascular device. According to some such embodiments, the length of segment 110 of the endovascular device is at least 208.7% of the length of the working segment 120 of the endovascular device. According to some such embodiments, the length of segment 110 of the endovascular device is at least 208.8% of the length of the working segment 120 of the endovascular device. According to some such embodiments, the length of segment 110 of the endovascular device is at least 208.9% of the length of the working segment 120 of the endovascular device. According to some such embodiments, the length of segment 110 of the endovascular device is at least 209% of the length of the working segment 120 of the endovascular device. According to some such embodiments, the length of segment 110 of the endovascular device is at least 209.1% of the length of the working segment 120 of the endovascular device. According to some such embodiments, the length of segment 110 of the endovascular device is at least 209.2% of the length of the working segment 120 of the endovascular device. According to some such embodiments, the length of segment 110 of the endovascular device is at least 209.3% of the length of the working segment 120 of the endovascular device. According to some such embodiments, the length of segment 110 of the endovascular device is at least 209.4% of the length of the working segment 120 of the endovascular device. According to some such embodiments, the length of segment 110 of the endovascular device is at least 209.5% of the length of the working segment 120 of the endovascular device. According to some embodiments, the length of segment 110 of the endovascular device is at least 209.6% of the length of the working segment 120 of the endovascular device. According to some embodiments, the length of segment 110 of the endovascular device is at least 209.7% of the length of the working segment 120 of the endovascular device. According to some embodiments, the length of segment 110 of the endovascular device is at least 209.8% of the length of the working segment 120 of the endovascular device. According to some embodiments, the length of segment 110 of the endovascular device is at least 209.9% of the length of the working segment 120 of the endovascular device. According to some such embodiments, the length of segment 110 of the endovascular device is at least 210% of the length of the working segment 120 of the endovascular device. According to some such embodiments, the length of segment 110 of the endovascular device is at least 210.1% of the length of the working segment 120 of the endovascular device. According to some such embodiments, the length of segment 110 of the endovascular device is at least 210.2% of the length of the working segment 120 of the endovascular device. According to some such embodiments, the length of segment 110 of the endovascular device is at least 210.3% of the length of the working segment 120 of the endovascular device. According to some such embodiments, the length of segment 110 of the endovascular device is at least 210.4% of the length of the working segment 120 of the endovascular device. According to some such embodiments, the length of segment 110 of the endovascular device is at least 210.5% of the length of the working segment 120 of the endovascular device. According to some such embodiments, the length of segment 110 of the endovascular device is at least 210.6% of the length of the working segment 120 of the endovascular device. According to some such embodiments, the length of segment 110 of the endovascular device is at least 210.7% of the length of the working segment 120 of the endovascular device. According to some such embodiments, the length of segment 110 of the endovascular device is at least 210.8% of the length of the working segment 120 of the endovascular device. According to some such embodiments, the length of segment 110 of the endovascular device is at least 210.9% of the length of the working segment 120 of the endovascular device. According to some such embodiments, the length of segment 110 of the endovascular device is at least 211% of the length of the working segment 120 of the endovascular device. According to some such embodiments, the length of segment 110 of the endovascular device is at least 211.1% of the length of the working segment 120 of the endovascular device. According to some such embodiments, the length of segment 110 of the endovascular device is at least 211.2% of the length of the working segment 120 of the endovascular device. According to some such embodiments, the length of segment 110 of the endovascular device is at least 211.3% of the length of the working segment 120 of the endovascular device. According to some such embodiments, the length of segment 110 of the endovascular device is at least 211.4% of the length of the working segment 120 of the endovascular device. According to some such embodiments, the length of segment 110 of the endovascular device is at least 211.5% of the length of the working segment 120 of the endovascular device. According to some such embodiments, the length of segment 110 of the endovascular device is at least 211.6% of the length of the working segment 120 of the endovascular device. According to some such embodiments, the length of segment 110 of the endovascular device is at least 211.7% of the length of the working segment 120 of the endovascular device. According to some such embodiments, the length of segment 110 of the endovascular device is at least 211.8% of the length of the working segment 120 of the endovascular device. According to some such embodiments, the length of segment 110 of the endovascular device is at least 211.9% of the length of the working segment 120 of the endovascular device. According to some such embodiments, the length of segment 110 of the endovascular device is at least 212% of the length of the working segment 120 of the endovascular device. According to some such embodiments, the length of segment 110 of the endovascular device is at least 212.1% of the length of the working segment 120 of the endovascular device. According to some such embodiments, the length of segment 110 of the endovascular device is at least 212.2% of the length of the working segment 120 of the endovascular device. According to some such embodiments, the length of segment 110 of the endovascular device is at least 212.3% of the length of the working segment 120 of the endovascular device. According to some such embodiments, the length of segment 110 of the endovascular device is at least 212.4% of the length of the working segment 120 of the endovascular device. According to some such embodiments, the length of segment 110 of the endovascular device is at least 212.5% of the length of the working segment 120 of the endovascular device. According to some such embodiments, the length of segment 110 of the endovascular device is at least 212.6% of the length of the working segment 120 of the endovascular device. According to some such embodiments, the length of segment 110 of the endovascular device is at least 212.7% of the length of the working segment 120 of the endovascular device. According to some such embodiments, the length of segment 110 of the endovascular device is at least 212.8% of the length of the working segment 120 of the endovascular device. According to some such embodiments, the length of segment 110 of the endovascular device is at least 212.9% of the length of the working segment 120 of the endovascular device. According to some such embodiments, the length of segment 110 of the endovascular device is at least 213% of the length of the working segment 120 of the endovascular device. According to some such embodiments, the length of segment 110 of the endovascular device is at least 213.1% of the length of the working segment 120 of the endovascular device. According to some such embodiments, the length of segment 110 of the endovascular device is at least 213.2% of the length of the working segment 120 of the endovascular device. According to some such embodiments, the length of segment 110 of the endovascular device is at least 213.3% of the length of the working segment 120 of the endovascular device. According to some such embodiments, the length of segment 110 of the endovascular device is at least 213.4% of the length of the working segment 120 of the endovascular device. According to some such embodiments, the length of segment 110 of the endovascular device is at least 213.5% of the length of the working segment 120 of the endovascular device. According to some such embodiments, the length of segment 110 of the endovascular device is at least 213.6% of the length of the working segment 120 of the endovascular device. According to some such embodiments, the length of segment 110 of the endovascular device is at least 213.7% of the length of the working segment 120 of the endovascular device. According to some such embodiments, the length of segment 110 of the endovascular device is at least 213.8% of the length of the working segment 120 of the endovascular device. According to some such embodiments, the length of segment 110 of the endovascular device is at least 213.9% of the length of the working segment 120 of the endovascular device. According to some such embodiments, the length of segment 110 of the endovascular device is at least 214% of the length of the working segment 120 of the endovascular device. According to some such embodiments, the length of segment 110 of the endovascular device is at least 214.1% of the length of the working segment 120 of the endovascular device. According to some such embodiments, the length of segment 110 of the endovascular device is at least 214.2% of the length of the working segment 120 of the endovascular device. According to some such embodiments, the length of segment 110 of the endovascular device is at least 214.3% of the length of the working segment 120 of the endovascular device. According to some such embodiments, the length of segment 110 of the endovascular device is at least 214.4% of the length of the working segment 120 of the endovascular device. According to some such embodiments, the length of segment 110 of the endovascular device is at least 214.5% of the length of the working segment 120 of the endovascular device. According to some such embodiments, the length of segment 110 of the endovascular device is at least 214.6% of the length of the working segment 120 of the endovascular device. According to some such embodiments, the length of segment 110 of the endovascular device is at least 214.7% of the length of the working segment 120 of the endovascular device. According to some such embodiments, the length of segment 110 of the endovascular device is at least 214.8% of the length of the working segment 120 of the endovascular device. According to some such embodiments, the length of segment 110 of the endovascular device is at least 214.9% of the length of the working segment 120 of the endovascular device. According to some such embodiments, the length of segment 110 of the endovascular device is at least 215% of the length of the working segment 120 of the endovascular device. According to some such embodiments, the length of segment 110 of the endovascular device is at least 215.1% of the length of the working segment 120 of the endovascular device. According to some such embodiments, the length of segment 110 of the endovascular device is at least 215.2% of the length of the working segment 120 of the endovascular device. According to some such embodiments, the length of segment 110 of the endovascular device is at least 215.3% of the length of the working segment 120 of the endovascular device. According to some such embodiments, the length of segment 110 of the endovascular device is at least 215.4% of the length of the working segment 120 of the endovascular device. According to some such embodiments, the length of segment 110 of the endovascular device is at least 215.5% of the length of the working segment 120 of the endovascular device. According to some such embodiments, the length of segment 110 of the endovascular device is at least 215.6% of the length of the working segment 120 of the endovascular device. According to some such embodiments, the length of segment 110 of the endovascular device is at least 215.7% of the length of the working segment 120 of the endovascular device. According to some such embodiments, the length of segment 110 of the endovascular device is at least 215.8% of the length of the working segment 120 of the endovascular device. According to some such embodiments, the length of segment 110 of the endovascular device is at least 215.9% of the length of the working segment 120 of the endovascular device. According to some such embodiments, the length of segment 110 of the endovascular device is at least 216% of the length of the working segment 120 of the endovascular device. According to some such embodiments, the length of segment 110 of the endovascular device is at least 216.1% of the length of the working segment 120 of the endovascular device. According to some such embodiments, the length of segment 110 of the endovascular device is at least 216.2% of the length of the working segment 120 of the endovascular device. According to some such embodiments, the length of segment 110 of the endovascular device is at least 216.3% of the length of the working segment 120 of the endovascular device. According to some such embodiments, the length of segment 110 of the endovascular device is at least 216.4% of the length of the working segment 120 of the endovascular device. According to some such embodiments, the length of segment 110 of the endovascular device is at least 216.5% of the length of the working segment 120 of the endovascular device. According to some such embodiments, the length of segment 110 of the endovascular device is at least 216.6% of the length of the working segment 120 of the endovascular device. According to some such embodiments, the length of segment 110 of the endovascular device is at least 216.7% of the length of the working segment 120 of the endovascular device. According to some such embodiments, the length of segment 110 of the endovascular device is at least 216.8% of the length of the working segment 120 of the endovascular device. According to some such embodiments, the length of segment 110 of the endovascular device is at least 216.9% of the length of the working segment 120 of the endovascular device. According to some such embodiments, the length of segment 110 of the endovascular device is at least 217% of the length of the working segment 120 of the endovascular device. According to some such embodiments, the length of segment 110 of the endovascular device is at least 217.1% of the length of the working segment 120 of the endovascular device. According to some such embodiments, the length of segment 110 of the endovascular device is at least 217.2% of the length of the working segment 120 of the endovascular device. According to some such embodiments, the length of segment 110 of the endovascular device is at least 217.3% of the length of the working segment 120 of the endovascular device. According to some such embodiments, the length of segment 110 of the endovascular device is at least 217.4% of the length of the working segment 120 of the endovascular device. According to some such embodiments, the length of segment 110 of the endovascular device is at least 217.5% of the length of the working segment 120 of the endovascular device. According to some such embodiments, the length of segment 110 of the endovascular device is at least 217.6% of the length of the working segment 120 of the endovascular device. According to some such embodiments, the length of segment 110 of the endovascular device is at least 217.7% of the length of the working segment 120 of the endovascular device. According to some such embodiments, the length of segment 110 of the endovascular device is at least 217.8% of the length of the working segment 120 of the endovascular device. According to some such embodiments, the length of segment 110 of the endovascular device is at least 217.9% of the length of the working segment 120 of the endovascular device. According to some such embodiments, the length of segment 110 of the endovascular device is at least 218% of the length of the working segment 120 of the endovascular device. According to some such embodiments, the length of segment 110 of the endovascular device is at least 218.1% of the length of the working segment 120 of the endovascular device. According to some such embodiments, the length of segment 110 of the endovascular device is at least 218.2% of the length of the working segment 120 of the endovascular device. According to some such embodiments, the length of segment 110 of the endovascular device is at least 218.3% of the length of the working segment 120 of the endovascular device. According to some such embodiments, the length of segment 110 of the endovascular device is at least 218.4% of the length of the working segment 120 of the endovascular device. According to some such embodiments, the length of segment 110 of the endovascular device is at least 218.5% of the length of the working segment 120 of the endovascular device. According to some such embodiments, the length of segment 110 of the endovascular device is at least 218.6% of the length of the working segment 120 of the endovascular device. According to some such embodiments, the length of segment 110 of the endovascular device is at least 218.7% of the length of the working segment 120 of the endovascular device. According to some such embodiments, the length of segment 110 of the endovascular device is at least 218.8% of the length of the working segment 120 of the endovascular device. According to some such embodiments, the length of segment 110 of the endovascular device is at least 218.9% of the length of the working segment 120 of the endovascular device. According to some such embodiments, the length of segment 110 of the endovascular device is at least 219% of the length of the working segment 120 of the endovascular device. According to some such embodiments, the length of segment 110 of the endovascular device is at least 219.1% of the length of the working segment 120 of the endovascular device. According to some such embodiments, the length of segment 110 of the endovascular device is at least 219.2% of the length of the working segment 120 of the endovascular device. According to some such embodiments, the length of segment 110 of the endovascular device is at least 219.3% of the length of the working segment 120 of the endovascular device. According to some such embodiments, the length of segment 110 of the endovascular device is at least 219.4% of the length of the working segment 120 of the endovascular device. According to some such embodiments, the length of segment 110 of the endovascular device is at least 219.5% of the length of the working segment 120 of the endovascular device. According to some such embodiments, the length of segment 110 of the endovascular device is at least 219.6% of the length of the working segment 120 of the endovascular device. According to some such embodiments, the length of segment 110 of the endovascular device is at least 219.7% of the length of the working segment 120 of the endovascular device. According to some such embodiments, the length of segment 110 of the endovascular device is at least 219.8% of the length of the working segment 120 of the endovascular device. According to some such embodiments, the length of segment 110 of the endovascular device is at least 219.9% of the length of the working segment 120 of the endovascular device. According to some such embodiments, the length of segment 110 of the endovascular device is at least 220% of the length of the working segment 120 of the endovascular device.

According to some such embodiments, the length of segment 110 of the endovascular device is at least 220.1% of the length of the working segment 120 of the endovascular device. According to some such embodiments, the length of segment 110 of the endovascular device is at least 220.2% of the length of the working segment 120 of the endovascular device. According to some such embodiments, the length of segment 110 of the endovascular device is at least 220.3% of the length of the working segment 120 of the endovascular device. According to some such embodiments, the length of segment 110 of the endovascular device is at least 220.4% of the length of the working segment 120 of the endovascular device. According to some such embodiments, the length of segment 110 of the endovascular device is at least 220.5% of the length of the working segment 120 of the endovascular device. According to some such embodiments, the length of segment 110 of the endovascular device is at least 220.6% of the length of the working segment 120 of the endovascular device. According to some such embodiments, the length of segment 110 of the endovascular device is at least 220.7% of the length of the working segment 120 of the endovascular device. According to some such embodiments, the length of segment 110 of the endovascular device is at least 220.8% of the length of the working segment 120 of the endovascular device. According to some such embodiments, the length of segment 110 of the endovascular device is at least 220.9% of the length of the working segment 120 of the endovascular device. According to some such embodiments, the length of segment 110 of the endovascular device is at least 221% of the length of the working segment 120 of the endovascular device. According to some such embodiments, the length of segment 110 of the endovascular device is at least 221.1% of the length of the working segment 120 of the endovascular device. According to some such embodiments, the length of segment 110 of the endovascular device is at least 221.2% of the length of the working segment 120 of the endovascular device. According to some such embodiments, the length of segment 110 of the endovascular device is at least 221.3% of the length of the working segment 120 of the endovascular device. According to some such embodiments, the length of segment 110 of the endovascular device is at least 221.4% of the length of the working segment 120 of the endovascular device. According to some such embodiments, the length of segment 110 of the endovascular device is at least 221.5% of the length of the working segment 120 of the endovascular device. According to some such embodiments, the length of segment 110 of the endovascular device is at least 221.6% of the length of the working segment 120 of the endovascular device. According to some such embodiments, the length of segment 110 of the endovascular device is at least 221.7% of the length of the working segment 120 of the endovascular device. According to some such embodiments, the length of segment 110 of the endovascular device is at least 221.8% of the length of the working segment 120 of the endovascular device. According to some such embodiments, the length of segment 110 of the endovascular device is at least 221.9% of the length of the working segment 120 of the endovascular device. According to some such embodiments, the length of segment 110 of the endovascular device is at least 222% of the length of the working segment 120 of the endovascular device. According to some such embodiments, the length of segment 110 of the endovascular device is at least 222.1% of the length of the working segment 120 of the endovascular device. According to some such embodiments, the length of segment 110 of the endovascular device is at least 222.2% of the length of the working segment 120 of the endovascular device. According to some such embodiments, the length of segment 110 of the endovascular device is at least 222.3% of the length of the working segment 120 of the endovascular device. According to some such embodiments, the length of segment 110 of the endovascular device is at least 222.4% of the length of the working segment 120 of the endovascular device. According to some such embodiments, the length of segment 110 of the endovascular device is at least 222.5% of the length of the working segment 120 of the endovascular device. According to some such embodiments, the length of segment 110 of the endovascular device is at least 222.6% of the length of the working segment 120 of the endovascular device. According to some such embodiments, the length of segment 110 of the endovascular device is at least 222.7% of the length of the working segment 120 of the endovascular device. According to some such embodiments, the length of segment 110 of the endovascular device is at least 222.8% of the length of the working segment 120 of the endovascular device. According to some such embodiments, the length of segment 110 of the endovascular device is at least 222.9% of the length of the working segment 120 of the endovascular device. According to some such embodiments, the length of segment 110 of the endovascular device is at least 223% of the length of the working segment 120 of the endovascular device. According to some such embodiments, the length of segment 110 of the endovascular device is at least 223.1% of the length of the working segment 120 of the endovascular device. According to some such embodiments, the length of segment 110 of the endovascular device is at least 223.2% of the length of the working segment 120 of the endovascular device. According to some such embodiments, the length of segment 110 of the endovascular device is at least 223.3% of the length of the working segment 120 of the endovascular device. According to some such embodiments, the length of segment 110 of the endovascular device is at least 223.4% of the length of the working segment 120 of the endovascular device. According to some such embodiments, the length of segment 110 of the endovascular device is at least 223.5% of the length of the working segment 120 of the endovascular device. According to some such embodiments, the length of segment 110 of the endovascular device is at least 223.6% of the length of the working segment 120 of the endovascular device. According to some such embodiments, the length of segment 110 of the endovascular device is at least 223.7% of the length of the working segment 120 of the endovascular device. According to some such embodiments, the length of segment 110 of the endovascular device is at least 223.8% of the length of the working segment 120 of the endovascular device. According to some such embodiments, the length of segment 110 of the endovascular device is at least 223.9% of the length of the working segment 120 of the endovascular device. According to some such embodiments, the length of segment 110 of the endovascular device is at least 224% of the length of the working segment 120 of the endovascular device. According to some such embodiments, the length of segment 110 of the endovascular device is at least 224.1% of the length of the working segment 120 of the endovascular device. According to some such embodiments, the length of segment 110 of the endovascular device is at least 224.2% of the length of the working segment 120 of the endovascular device. According to some such embodiments, the length of segment 110 of the endovascular device is at least 224.3% of the length of the working segment 120 of the endovascular device. According to some such embodiments, the length of segment 110 of the endovascular device is at least 224.4% of the length of the working segment 120 of the endovascular device. According to some such embodiments, the length of segment 110 of the endovascular device is at least 224.5% of the length of the working segment 120 of the endovascular device. According to some such embodiments, the length of segment 110 of the endovascular device is at least 224.6% of the length of the working segment 120 of the endovascular device. According to some such embodiments, the length of segment 110 of the endovascular device is at least 224.7% of the length of the working segment 120 of the endovascular device. According to some such embodiments, the length of segment 110 of the endovascular device is at least 224.8% of the length of the working segment 120 of the endovascular device. According to some such embodiments, the length of segment 110 of the endovascular device is at least 224.9% of the length of the working segment 120 of the endovascular device. According to some such embodiments, the length of segment 110 of the endovascular device is at least 225% of the length of the working segment 120 of the endovascular device.

According to some such embodiments, the length of segment 110 of the endovascular device is at least 225.1% of the length of the working segment 120 of the endovascular device. According to some such embodiments, the length of segment 110 of the endovascular device is at least 125.2% of the length of the working segment 120 of the endovascular device. According to some such embodiments, the length of segment 110 of the endovascular device is at least 225.3% of the length of the working segment 120 of the endovascular device. According to some such embodiments, the length of segment 110 of the endovascular device is at least 225.4% of the length of the working segment 120 of the endovascular device. According to some such embodiments, the length of segment 110 of the endovascular device is at least 225.5% of the length of the working segment 120 of the endovascular device. According to some such embodiments, the length of segment 110 of the endovascular device is at least 225.6% of the length of the working segment 120 of the endovascular device. According to some such embodiments, the length of segment 110 of the endovascular device is at least 225.7% of the length of the working segment 120 of the endovascular device. According to some such embodiments, the length of segment 110 of the endovascular device is at least 225.8% of the length of the working segment 120 of the endovascular device. According to some such embodiments, the length of segment 110 of the endovascular device is at least 225.9% of the length of the working segment 120 of the endovascular device. According to some such embodiments, the length of segment 110 of the endovascular device is at least 226% of the length of the working segment 120 of the endovascular device. According to some such embodiments, the length of segment 110 of the endovascular device is at least 226.1% of the length of the working segment 120 of the endovascular device. According to some such embodiments, the length of segment 110 of the endovascular device is at least 226.2% of the length of the working segment 120 of the endovascular device. According to some such embodiments, the length of segment 110 of the endovascular device is at least 226.3% of the length of the working segment 120 of the endovascular device. According to some such embodiments, the length of segment 110 of the endovascular device is at least 226.4% of the length of the working segment 120 of the endovascular device. According to some such embodiments, the length of segment 110 of the endovascular device is at least 226.5% of the length of the working segment 120 of the endovascular device. According to some such embodiments, the length of segment 110 of the endovascular device is at least 226.6% of the length of the working segment 120 of the endovascular device. According to some such embodiments, the length of segment 110 of the endovascular device is at least 226.7% of the length of the working segment 120 of the endovascular device. According to some such embodiments, the length of segment 110 of the endovascular device is at least 226.8% of the length of the working segment 120 of the endovascular device. According to some such embodiments, the length of segment 110 of the endovascular device is at least 226.9% of the length of the working segment 120 of the endovascular device. According to some such embodiments, the length of segment 110 of the endovascular device is at least 227% of the length of the working segment 120 of the endovascular device. According to some such embodiments, the length of segment 110 of the endovascular device is at least 227.1% of the length of the working segment 120 of the endovascular device. According to some such embodiments, the length of segment 110 of the endovascular device is at least 227.2% of the length of the working segment 120 of the endovascular device. According to some such embodiments, the length of segment 110 of the endovascular device is at least 227.3% of the length of the working segment 120 of the endovascular device. According to some such embodiments, the length of segment 110 of the endovascular device is at least 227.4% of the length of the working segment 120 of the endovascular device. According to some such embodiments, the length of segment 110 of the endovascular device is at least 227.5% of the length of the working segment 120 of the endovascular device. According to some such embodiments, the length of segment 110 of the endovascular device is at least 227.6% of the length of the working segment 120 of the endovascular device. According to some such embodiments, the length of segment 110 of the endovascular device is at least 227.7% of the length of the working segment 120 of the endovascular device. According to some such embodiments, the length of segment 110 of the endovascular device is at least 227.8% of the length of the working segment 120 of the endovascular device. According to some such embodiments, the length of segment 110 of the endovascular device is at least 227.9% of the length of the working segment 120 of the endovascular device. According to some such embodiments, the length of segment 110 of the endovascular device is at least 228% of the length of the working segment 120 of the endovascular device. According to some such embodiments, the length of segment 110 of the endovascular device is at least 228.1% of the length of the working segment 120 of the endovascular device. According to some such embodiments, the length of segment 110 of the endovascular device is at least 228.2% of the length of the working segment 120 of the endovascular device. According to some such embodiments, the length of segment 110 of the endovascular device is at least 228.3% of the length of the working segment 120 of the endovascular device. According to some such embodiments, the length of segment 110 of the endovascular device is at least 228.4% of the length of the working segment 120 of the endovascular device. According to some such embodiments, the length of segment 110 of the endovascular device is at least 228.5% of the length of the working segment 120 of the endovascular device. According to some such embodiments, the length of segment 110 of the endovascular device is at least 228.6% of the length of the working segment 120 of the endovascular device. According to some such embodiments, the length of segment 110 of the endovascular device is at least 228.7% of the length of the working segment 120 of the endovascular device. According to some such embodiments, the length of segment 110 of the endovascular device is at least 228.8% of the length of the working segment 120 of the endovascular device. According to some such embodiments, the length of segment 110 of the endovascular device is at least 228.9% of the length of the working segment 120 of the endovascular device. According to some such embodiments, the length of segment 110 of the endovascular device is at least 229% of the length of the working segment 120 of the endovascular device. According to some such embodiments, the length of segment 110 of the endovascular device is at least 229.1% of the length of the working segment 120 of the endovascular device. According to some such embodiments, the length of segment 110 of the endovascular device is at least 229.2% of the length of the working segment 120 of the endovascular device. According to some such embodiments, the length of segment 110 of the endovascular device is at least 229.3% of the length of the working segment 120 of the endovascular device. According to some such embodiments, the length of segment 110 of the endovascular device is at least 229.4% of the length of the working segment 120 of the endovascular device. According to some such embodiments, the length of segment 110 of the endovascular device is at least 229.5% of the length of the working segment 120 of the endovascular device. According to some such embodiments, the length of segment 110 of the endovascular device is at least 229.6% of the length of the working segment 120 of the endovascular device. According to some such embodiments, the length of segment 110 of the endovascular device is at least 229.7% of the length of the working segment 120 of the endovascular device. According to some such embodiments, the length of segment 110 of the endovascular device is at least 229.8% of the length of the working segment 120 of the endovascular device. According to some such embodiments, the length of segment 110 of the endovascular device is at least 229.9% of the length of the working segment 120 of the endovascular device. According to some such embodiments, the length of segment 110 of the endovascular device is at least 230% of the length of the working segment 120 of the endovascular device. According to some such embodiments, the length of segment 110 of the endovascular device is at least 230.1% of the length of the working segment 120 of the endovascular device. According to some such embodiments, the length of segment 110 of the endovascular device is at least 230.2% of the length of the working segment 120 of the endovascular device. According to some such embodiments, the length of segment 110 of the endovascular device is at least 230.3% of the length of the working segment 120 of the endovascular device. According to some such embodiments, the length of segment 110 of the endovascular device is at least 230.4% of the length of the working segment 120 of the endovascular device. According to some such embodiments, the length of segment 110 of the endovascular device is at least 230.5% of the length of the working segment 120 of the endovascular device. According to some such embodiments, the length of segment 110 of the endovascular device is at least 230.6% of the length of the working segment 120 of the endovascular device. According to some such embodiments, the length of segment 110 of the endovascular device is at least 230.7% of the length of the working segment 120 of the endovascular device. According to some such embodiments, the length of segment 110 of the endovascular device is at least 230.8% of the length of the working segment 120 of the endovascular device. According to some such embodiments, the length of segment 110 of the endovascular device is at least 230.9% of the length of the working segment 120 of the endovascular device.

According to some such embodiments, the length of segment 110 of the endovascular device is at least 231% of the length of the working segment 120 of the endovascular device. According to some such embodiments, the length of segment 110 of the endovascular device is at least 231.1% of the length of the working segment 120 of the endovascular device. According to some such embodiments, the length of segment 110 of the endovascular device is at least 231.2% of the length of the working segment 120 of the endovascular device. According to some such embodiments, the length of segment 110 of the endovascular device is at least 231.3% of the length of the working segment 120 of the endovascular device. According to some such embodiments, the length of segment 110 of the endovascular device is at least 231.4% of the length of the working segment 120 of the endovascular device. According to some such embodiments, the length of segment 110 of the endovascular device is at least 231.5% of the length of the working segment 120 of the endovascular device. According to some such embodiments, the length of segment 110 of the endovascular device is at least 231.6% of the length of the working segment 120 of the endovascular device. According to some such embodiments, the length of segment 110 of the endovascular device is at least 231.7% of the length of the working segment 120 of the endovascular device. According to some such embodiments, the length of segment 110 of the endovascular device is at least 231.8% of the length of the working segment 120 of the endovascular device. According to some such embodiments, the length of segment 110 of the endovascular device is at least 231.9% of the length of the working segment 120 of the endovascular device. According to some such embodiments, the length of segment 110 of the endovascular device is at least 232% of the length of the working segment 120 of the endovascular device. According to some such embodiments, the length of segment 110 of the endovascular device is at least 232.1% of the length of the working segment 120 of the endovascular device. According to some such embodiments, the length of segment 110 of the endovascular device is at least 232.2% of the length of the working segment 120 of the endovascular device. According to some such embodiments, the length of segment 110 of the endovascular device is at least 232.3% of the length of the working segment 120 of the endovascular device. According to some such embodiments, the length of segment 110 of the endovascular device is at least 232.4% of the length of the working segment 120 of the endovascular device. According to some such embodiments, the length of segment 110 of the endovascular device is at least 232.5% of the length of the working segment 120 of the endovascular device. According to some such embodiments, the length of segment 110 of the endovascular device is at least 232.6% of the length of the working segment 120 of the endovascular device. According to some such embodiments, the length of segment 110 of the endovascular device is at least 232.7% of the length of the working segment 120 of the endovascular device. According to some such embodiments, the length of segment 110 of the endovascular device is at least 232.8% of the length of the working segment 120 of the endovascular device. According to some such embodiments, the length of segment 110 of the endovascular device is at least 232.9% of the length of the working segment 120 of the endovascular device. According to some such embodiments, the length of segment 110 of the endovascular device is at least 232% of the length of the working segment 120 of the endovascular device. According to some such embodiments, the length of segment 110 of the endovascular device is at least 233.1% of the length of the working segment 120 of the endovascular device. According to some such embodiments, the length of segment 110 of the endovascular device is at least 233.2% of the length of the working segment 120 of the endovascular device. According to some such embodiments, the length of segment 110 of the endovascular device is at least 233.3% of the length of the working segment 120 of the endovascular device. According to some such embodiments, the length of segment 110 of the endovascular device is at least 233.4% of the length of the working segment 120 of the endovascular device. According to some such embodiments, the length of segment 110 of the endovascular device is at least 233.5% of the length of the working segment 120 of the endovascular device. According to some such embodiments, the length of segment 110 of the endovascular device is at least 233.6% of the length of the working segment 120 of the endovascular device. According to some such embodiments, the length of segment 110 of the endovascular device is at least 233.7% of the length of the working segment 120 of the endovascular device. According to some such embodiments, the length of segment 110 of the endovascular device is at least 233.8% of the length of the working segment 120 of the endovascular device. According to some such embodiments, the length of segment 110 of the endovascular device is at least 233.9% of the length of the working segment 120 of the endovascular device. According to some such embodiments, the length of segment 110 of the endovascular device is at least 234% of the length of the working segment 120 of the endovascular device. According to some such embodiments, the length of segment 110 of the endovascular device is at least 234.1% of the length of the working segment 120 of the endovascular device. According to some such embodiments, the length of segment 110 of the endovascular device is at least 234.2% of the length of the working segment 120 of the endovascular device. According to some such embodiments, the length of segment 110 of the endovascular device is at least 234.3% of the length of the working segment 120 of the endovascular device. According to some such embodiments, the length of segment 110 of the endovascular device is at least 234.4% of the length of the working segment 120 of the endovascular device. According to some such embodiments, the length of segment 110 of the endovascular device is at least 234.5% of the length of the working segment 120 of the endovascular device. According to some such embodiments, the length of segment 110 of the endovascular device is at least 234.6% of the length of the working segment 120 of the endovascular device. According to some such embodiments, the length of segment 110 of the endovascular device is at least 234.7% of the length of the working segment 120 of the endovascular device. According to some such embodiments, the length of segment 110 of the endovascular device is at least 234.8% of the length of the working segment 120 of the endovascular device. According to some such embodiments, the length of segment 110 of the endovascular device is at least 234.9% of the length of the working segment 120 of the endovascular device. According to some such embodiments, the length of segment 110 of the endovascular device is at least 235% of the length of the working segment 120 of the endovascular device. According to some such embodiments, the length of segment 110 of the endovascular device is at least 235.1% of the length of the working segment 120 of the endovascular device. According to some such embodiments, the length of segment 110 of the endovascular device is at least 235.2% of the length of the working segment 120 of the endovascular device. According to some such embodiments, the length of segment 110 of the endovascular device is at least 235.3% of the length of the working segment 120 of the endovascular device. According to some such embodiments, the length of segment 110 of the endovascular device is at least 235.4% of the length of the working segment 120 of the endovascular device. According to some such embodiments, the length of segment 110 of the endovascular device is at least 235.5% of the length of the working segment 120 of the endovascular device. According to some such embodiments, the length of segment 110 of the endovascular device is at least 235.6% of the length of the working segment 120 of the endovascular device. According to some such embodiments, the length of segment 110 of the endovascular device is at least 235.7% of the length of the working segment 120 of the endovascular device. According to some such embodiments, the length of segment 110 of the endovascular device is at least 235.8% of the length of the working segment 120 of the endovascular device. According to some such embodiments, the length of segment 110 of the endovascular device is at least 235.9% of the length of the working segment 120 of the endovascular device. According to some such embodiments, the length of segment 110 of the endovascular device is at least 236% of the length of the working segment 120 of the endovascular device. According to some such embodiments, the length of segment 110 of the endovascular device is at least 236.1% of the length of the working segment 120 of the endovascular device. According to some such embodiments, the length of segment 110 of the endovascular device is at least 236.2% of the length of the working segment 120 of the endovascular device. According to some such embodiments, the length of segment 110 of the endovascular device is at least 236.3% of the length of the working segment 120 of the endovascular device. According to some such embodiments, the length of segment 110 of the endovascular device is at least 236.4% of the length of the working segment 120 of the endovascular device. According to some such embodiments, the length of segment 110 of the endovascular device is at least 236.5% of the length of the working segment 120 of the endovascular device. According to some such embodiments, the length of segment 110 of the endovascular device is at least 236.6% of the length of the working segment 120 of the endovascular device. According to some such embodiments, the length of segment 110 of the endovascular device is at least 236.7% of the length of the working segment 120 of the endovascular device. According to some such embodiments, the length of segment 110 of the endovascular device is at least 236.8% of the length of the working segment 120 of the endovascular device. According to some such embodiments, the length of segment 110 of the endovascular device is at least 236.9% of the length of the working segment 120 of the endovascular device. According to some such embodiments, the length of segment 110 of the endovascular device is at least 237% of the length of the working segment 120 of the endovascular device. According to some such embodiments, the length of segment 110 of the endovascular device is at least 237.1% of the length of the working segment 120 of the endovascular device. According to some such embodiments, the length of segment 110 of the endovascular device is at least 237.2% of the length of the working segment 120 of the endovascular device. According to some such embodiments, the length of segment 110 of the endovascular device is at least 237.3% of the length of the working segment 120 of the endovascular device. According to some such embodiments, the length of segment 110 of the endovascular device is at least 237.4% of the length of the working segment 120 of the endovascular device. According to some such embodiments, the length of segment 110 of the endovascular device is at least 237.5% of the length of the working segment 120 of the endovascular device. According to some such embodiments, the length of segment 110 of the endovascular device is at least 237.6% of the length of the working segment 120 of the endovascular device. According to some such embodiments, the length of segment 110 of the endovascular device is at least 237.7% of the length of the working segment 120 of the endovascular device. According to some such embodiments, the length of segment 110 of the endovascular device is at least 237.8% of the length of the working segment 120 of the endovascular device. According to some such embodiments, the length of segment 110 of the endovascular device is at least 237.9% of the length of the working segment 120 of the endovascular device. According to some such embodiments, the length of segment 110 of the endovascular device is at least 238% of the length of the working segment 120 of the endovascular device. According to some such embodiments, the length of segment 110 of the endovascular device is at least 238.1% of the length of the working segment 120 of the endovascular device. According to some such embodiments, the length of segment 110 of the endovascular device is at least 238.2% of the length of the working segment 120 of the endovascular device. According to some such embodiments, the length of segment 110 of the endovascular device is at least 238.3% of the length of the working segment 120 of the endovascular device. According to some such embodiments, the length of segment 110 of the endovascular device is at least 238.4% of the length of the working segment 120 of the endovascular device. According to some such embodiments, the length of segment 110 of the endovascular device is at least 238.5% of the length of the working segment 120 of the endovascular device. According to some such embodiments, the length of segment 110 of the endovascular device is at least 238.6% of the length of the working segment 120 of the endovascular device. According to some such embodiments, the length of segment 110 of the endovascular device is at least 238.7% of the length of the working segment 120 of the endovascular device. According to some such embodiments, the length of segment 110 of the endovascular device is at least 238.8% of the length of the working segment 120 of the endovascular device. According to some such embodiments, the length of segment 110 of the endovascular device is at least 238.9% of the length of the working segment 120 of the endovascular device. According to some such embodiments, the length of segment 110 of the endovascular device is at least 239% of the length of the working segment 120 of the endovascular device. According to some such embodiments, the length of segment 110 of the endovascular device is at least 239.1% of the length of the working segment 120 of the endovascular device. According to some such embodiments, the length of segment 110 of the endovascular device is at least 239.2% of the length of the working segment 120 of the endovascular device. According to some such embodiments, the length of segment 110 of the endovascular device is at least 239.3% of the length of the working segment 120 of the endovascular device. According to some such embodiments, the length of segment 110 of the endovascular device is at least 239.4% of the length of the working segment 120 of the endovascular device. According to some such embodiments, the length of segment 110 of the endovascular device is at least 239.5% of the length of the working segment 120 of the endovascular device. According to some such embodiments, the length of segment 110 of the endovascular device is at least 239.6% of the length of the working segment 120 of the endovascular device. According to some such embodiments, the length of segment 110 of the endovascular device is at least 239.7% of the length of the working segment 120 of the endovascular device. According to some such embodiments, the length of segment 110 of the endovascular device is at least 239.8% of the length of the working segment 120 of the endovascular device. According to some such embodiments, the length of segment 110 of the endovascular device is at least 239.9% of the length of the working segment 120 of the endovascular device.

According to some such embodiments, the length of segment 110 of the endovascular device is at least 240% of the length of the working segment 120 of the endovascular device. According to some such embodiments, the length of segment 110 of the endovascular device is at least 240.1% of the length of the working segment 120 of the endovascular device. According to some such embodiments, the length of segment 110 of the endovascular device is at least 240.2% of the length of the working segment 120 of the endovascular device. According to some such embodiments, the length of segment 110 of the endovascular device is at least 240.3% of the length of the working segment 120 of the endovascular device. According to some such embodiments, the length of segment 110 of the endovascular device is at least 240.4% of the length of the working segment 120 of the endovascular device. According to some such embodiments, the length of segment 110 of the endovascular device is at least 240.5% of the length of the working segment 120 of the endovascular device. According to some such embodiments, the length of segment 110 of the endovascular device is at least 240.6% of the length of the working segment 120 of the endovascular device. According to some such embodiments, the length of segment 110 of the endovascular device is at least 240.7% of the length of the working segment 120 of the endovascular device. According to some such embodiments, the length of segment 110 of the endovascular device is at least 240.8% of the length of the working segment 120 of the endovascular device. According to some such embodiments, the length of segment 110 of the endovascular device is at least 240.9% of the length of the working segment 120 of the endovascular device. According to some such embodiments, the length of segment 110 of the endovascular device is at least 241% of the length of the working segment 120 of the endovascular device. According to some such embodiments, the length of segment 110 of the endovascular device is at least 241.1% of the length of the working segment 120 of the endovascular device. According to some such embodiments, the length of segment 110 of the endovascular device is at least 241.2% of the length of the working segment 120 of the endovascular device. According to some such embodiments, the length of segment 110 of the endovascular device is at least 241.3% of the length of the working segment 120 of the endovascular device. According to some such embodiments, the length of segment 110 of the endovascular device is at least 241.4% of the length of the working segment 120 of the endovascular device. According to some such embodiments, the length of segment 110 of the endovascular device is at least 241.5% of the length of the working segment 120 of the endovascular device. According to some such embodiments, the length of segment 110 of the endovascular device is at least 241.6% of the length of the working segment 120 of the endovascular device. According to some such embodiments, the length of segment 110 of the endovascular device is at least 241.7% of the length of the working segment 120 of the endovascular device. According to some such embodiments, the length of segment 110 of the endovascular device is at least 241.8% of the length of the working segment 120 of the endovascular device. According to some such embodiments, the length of segment 110 of the endovascular device is at least 241.9% of the length of the working segment 120 of the endovascular device. According to some such embodiments, the length of segment 110 of the endovascular device is at least 242% of the length of the working segment 120 of the endovascular device. According to some such embodiments, the length of segment 110 of the endovascular device is at least 242.1% of the length of the working segment 120 of the endovascular device. According to some such embodiments, the length of segment 110 of the endovascular device is at least 242.2% of the length of the working segment 120 of the endovascular device. According to some such embodiments, the length of segment 110 of the endovascular device is at least 242.3% of the length of the working segment 120 of the endovascular device. According to some such embodiments, the length of segment 110 of the endovascular device is at least 242.4% of the length of the working segment 120 of the endovascular device. According to some such embodiments, the length of segment 110 of the endovascular device is at least 242.5% of the length of the working segment 120 of the endovascular device. According to some such embodiments, the length of segment 110 of the endovascular device is at least 242.6% of the length of the working segment 120 of the endovascular device. According to some such embodiments, the length of segment 110 of the endovascular device is at least 242.7% of the length of the working segment 120 of the endovascular device. According to some such embodiments, the length of segment 110 of the endovascular device is at least 242.8% of the length of the working segment 120 of the endovascular device. According to some such embodiments, the length of segment 110 of the endovascular device is at least 242.9% of the length of the working segment 120 of the endovascular device. According to some such embodiments, the length of segment 110 of the endovascular device is at least 243% of the length of the working segment 120 of the endovascular device. According to some such embodiments, the length of segment 110 of the endovascular device is at least 243.1% of the length of the working segment 120 of the endovascular device. According to some such embodiments, the length of segment 110 of the endovascular device is at least 243.2% of the length of the working segment 120 of the endovascular device. According to some such embodiments, the length of segment 110 of the endovascular device is at least 243.3% of the length of the working segment 120 of the endovascular device. According to some such embodiments, the length of segment 110 of the endovascular device is at least 243.4% of the length of the working segment 120 of the endovascular device. According to some such embodiments, the length of segment 110 of the endovascular device is at least 243.5% of the length of the working segment 120 of the endovascular device. According to some such embodiments, the length of segment 110 of the endovascular device is at least 243.6% of the length of the working segment 120 of the endovascular device. According to some such embodiments, the length of segment 110 of the endovascular device is at least 243.7% of the length of the working segment 120 of the endovascular device. According to some such embodiments, the length of segment 110 of the endovascular device is at least 243.8% of the length of the working segment 120 of the endovascular device. According to some such embodiments, the length of segment 110 of the endovascular device is at least 243.9% of the length of the working segment 120 of the endovascular device. According to some such embodiments, the length of segment 110 of the endovascular device is at least 244% of the length of the working segment 120 of the endovascular device. According to some such embodiments, the length of segment 110 of the endovascular device is at least 244.1% of the length of the working segment 120 of the endovascular device. According to some such embodiments, the length of segment 110 of the endovascular device is at least 244.2% of the length of the working segment 120 of the endovascular device. According to some such embodiments, the length of segment 110 of the endovascular device is at least 244.3% of the length of the working segment 120 of the endovascular device. According to some such embodiments, the length of segment 110 of the endovascular device is at least 244.4% of the length of the working segment 120 of the endovascular device. According to some such embodiments, the length of segment 110 of the endovascular device is at least 244.5% of the length of the working segment 120 of the endovascular device. According to some such embodiments, the length of segment 110 of the endovascular device is at least 244.6% of the length of the working segment 120 of the endovascular device. According to some such embodiments, the length of segment 110 of the endovascular device is at least 244.7% of the length of the working segment 120 of the endovascular device. According to some such embodiments, the length of segment 110 of the endovascular device is at least 244.8% of the length of the working segment 120 of the endovascular device. According to some such embodiments, the length of segment 110 of the endovascular device is at least 244.9% of the length of the working segment 120 of the endovascular device. According to some such embodiments, the length of segment 110 of the endovascular device is at least 245% of the length of the working segment 120 of the endovascular device. According to some such embodiments, the length of segment 110 of the endovascular device is at least 245.1% of the length of the working segment 120 of the endovascular device. According to some such embodiments, the length of segment 110 of the endovascular device is at least 245.2% of the length of the working segment 120 of the endovascular device. According to some such embodiments, the length of segment 110 of the endovascular device is at least 245.3% of the length of the working segment 120 of the endovascular device. According to some such embodiments, the length of segment 110 of the endovascular device is at least 245.4% of the length of the working segment 120 of the endovascular device. According to some such embodiments, the length of segment 110 of the endovascular device is at least 245.5% of the length of the working segment 120 of the endovascular device. According to some such embodiments, the length of segment 110 of the endovascular device is at least 245.6% of the length of the working segment 120 of the endovascular device. According to some such embodiments, the length of segment 110 of the endovascular device is at least 245.7% of the length of the working segment 120 of the endovascular device. According to some such embodiments, the length of segment 110 of the endovascular device is at least 245.8% of the length of the working segment 120 of the endovascular device. According to some such embodiments, the length of segment 110 of the endovascular device is at least 245.9% of the length of the working segment 120 of the endovascular device. According to some such embodiments, the length of segment 110 of the endovascular device is at least 246% of the length of the working segment 120 of the endovascular device. According to some such embodiments, the length of segment 110 of the endovascular device is at least 246.1% of the length of the working segment 120 of the endovascular device. According to some such embodiments, the length of segment 110 of the endovascular device is at least 246.2% of the length of the working segment 120 of the endovascular device. According to some such embodiments, the length of segment 110 of the endovascular device is at least 246.3% of the length of the working segment 120 of the endovascular device. According to some such embodiments, the length of segment 110 of the endovascular device is at least 246.4% of the length of the working segment 120 of the endovascular device. According to some such embodiments, the length of segment 110 of the endovascular device is at least 246.5% of the length of the working segment 120 of the endovascular device. According to some such embodiments, the length of segment 110 of the endovascular device is at least 246.6% of the length of the working segment 120 of the endovascular device. According to some such embodiments, the length of segment 110 of the endovascular device is at least 246.7% of the length of the working segment 120 of the endovascular device. According to some such embodiments, the length of segment 110 of the endovascular device is at least 246.8% of the length of the working segment 120 of the endovascular device. According to some such embodiments, the length of segment 110 of the endovascular device is at least 246.9% of the length of the working segment 120 of the endovascular device. According to some such embodiments, the length of segment 110 of the endovascular device is at least 247% of the length of the working segment 120 of the endovascular device. According to some such embodiments, the length of segment 110 of the endovascular device is at least 247.1% of the length of the working segment 120 of the endovascular device. According to some such embodiments, the length of segment 110 of the endovascular device is at least 247.2% of the length of the working segment 120 of the endovascular device. According to some such embodiments, the length of segment 110 of the endovascular device is at least 247.3% of the length of the working segment 120 of the endovascular device. According to some such embodiments, the length of segment 110 of the endovascular device is at least 247.4% of the length of the working segment 120 of the endovascular device. According to some such embodiments, the length of segment 110 of the endovascular device is at least 247.5% of the length of the working segment 120 of the endovascular device. According to some such embodiments, the length of segment 110 of the endovascular device is at least 247.6% of the length of the working segment 120 of the endovascular device. According to some such embodiments, the length of segment 110 of the endovascular device is at least 247.7% of the length of the working segment 120 of the endovascular device. According to some such embodiments, the length of segment 110 of the endovascular device is at least 247.8% of the length of the working segment 120 of the endovascular device. According to some such embodiments, the length of segment 110 of the endovascular device is at least 247.9% of the length of the working segment 120 of the endovascular device. According to some such embodiments, the length of segment 110 of the endovascular device is at least 248% of the length of the working segment 120 of the endovascular device. According to some such embodiments, the length of segment 110 of the endovascular device is at least 248.1% of the length of the working segment 120 of the endovascular device. According to some such embodiments, the length of segment 110 of the endovascular device is at least 248.2% of the length of the working segment 120 of the endovascular device. According to some such embodiments, the length of segment 110 of the endovascular device is at least 248.3% of the length of the working segment 120 of the endovascular device. According to some such embodiments, the length of segment 110 of the endovascular device is at least 248.4% of the length of the working segment 120 of the endovascular device. According to some such embodiments, the length of segment 110 of the endovascular device is at least 248.5% of the length of the working segment 120 of the endovascular device. According to some such embodiments, the length of segment 110 of the endovascular device is at least 248.6% of the length of the working segment 120 of the endovascular device. According to some such embodiments, the length of segment 110 of the endovascular device is at least 248.7% of the length of the working segment 120 of the endovascular device. According to some such embodiments, the length of segment 110 of the endovascular device is at least 248.8% of the length of the working segment 120 of the endovascular device. According to some such embodiments, the length of segment 110 of the endovascular device is at least 248.9% of the length of the working segment 120 of the endovascular device. According to some such embodiments, the length of segment 110 of the endovascular device is at least 249% of the length of the working segment 120 of the endovascular device. According to some such embodiments, the length of segment 110 of the endovascular device is at least 249.1% of the length of the working segment 120 of the endovascular device. According to some such embodiments, the length of segment 110 of the endovascular device is at least 249.2% of the length of the working segment 120 of the endovascular device. According to some such embodiments, the length of segment 110 of the endovascular device is at least 249.3% of the length of the working segment 120 of the endovascular device. According to some such embodiments, the length of segment 110 of the endovascular device is at least 249.4% of the length of the working segment 120 of the endovascular device. According to some such embodiments, the length of segment 110 of the endovascular device is at least 249.5% of the length of the working segment 120 of the endovascular device. According to some such embodiments, the length of segment 110 of the endovascular device is at least 249.6% of the length of the working segment 120 of the endovascular device. According to some such embodiments, the length of segment 110 of the endovascular device is at least 249.7% of the length of the working segment 120 of the endovascular device. According to some such embodiments, the length of segment 110 of the endovascular device is at least 249.8% of the length of the working segment 120 of the endovascular device. According to some such embodiments, the length of segment 110 of the endovascular device is at least 249.9% of the length of the working segment 120 of the endovascular device. According to some such embodiments, the length of segment 110 of the endovascular device is at least 250% of the length of the working segment 120 of the endovascular device.

According to some such embodiments, the length of segment 110 of the endovascular device is at least 250.1% of the length of the working segment 120 of the endovascular device. According to some such embodiments, the length of segment 110 of the endovascular device is at least 250.2% of the length of the working segment 120 of the endovascular device. According to some such embodiments, the length of segment 110 of the endovascular device is at least 250.3% of the length of the working segment 120 of the endovascular device. According to some such embodiments, the length of segment 110 of the endovascular device is at least 250.4% of the length of the working segment 120 of the endovascular device. According to some such embodiments, the length of segment 110 of the endovascular device is at least 250.5% of the length of the working segment 120 of the endovascular device. According to some such embodiments, the length of segment 110 of the endovascular device is at least 250.6% of the length of the working segment 120 of the endovascular device. According to some such embodiments, the length of segment 110 of the endovascular device is at least 250.7% of the length of the working segment 120 of the endovascular device. According to some such embodiments, the length of segment 110 of the endovascular device is at least 250.8% of the length of the working segment 120 of the endovascular device. According to some such embodiments, the length of segment 110 of the endovascular device is at least 250.9% of the length of the working segment 120 of the endovascular device. According to some such embodiments, the length of segment 110 of the endovascular device is at least 251% of the length of the working segment 120 of the endovascular device.

According to some such embodiments, the length of segment 110 of the endovascular device is at least 251.1% of the length of the working segment 120 of the endovascular device. According to some such embodiments, the length of segment 110 of the endovascular device is at least 251.2% of the length of the working segment 120 of the endovascular device. According to some such embodiments, the length of segment 110 of the endovascular device is at least 251.3% of the length of the working segment 120 of the endovascular device. According to some such embodiments, the length of segment 110 of the endovascular device is at least 251.4% of the length of the working segment 120 of the endovascular device. According to some such embodiments, the length of segment 110 of the endovascular device is at least 251.5% of the length of the working segment 120 of the endovascular device. According to some such embodiments, the length of segment 110 of the endovascular device is at least 251.6% of the length of the working segment 120 of the endovascular device. According to some such embodiments, the length of segment 110 of the endovascular device is at least 251.7% of the length of the working segment 120 of the endovascular device. According to some such embodiments, the length of segment 110 of the endovascular device is at least 251.8% of the length of the working segment 120 of the endovascular device. According to some such embodiments, the length of segment 110 of the endovascular device is at least 251.9% of the length of the working segment 120 of the endovascular device. According to some such embodiments, the length of segment 110 of the endovascular device is at least 252% of the length of the working segment 120 of the endovascular device. According to some such embodiments, the length of segment 110 of the endovascular device is at least 252.1% of the length of the working segment 120 of the endovascular device. According to some such embodiments, the length of segment 110 of the endovascular device is at least 252.2% of the length of the working segment 120 of the endovascular device. According to some such embodiments, the length of segment 110 of the endovascular device is at least 252.3% of the length of the working segment 120 of the endovascular device. According to some such embodiments, the length of segment 110 of the endovascular device is at least 252.4% of the length of the working segment 120 of the endovascular device. According to some such embodiments, the length of segment 110 of the endovascular device is at least 252.5% of the length of the working segment 120 of the endovascular device. According to some such embodiments, the length of segment 110 of the endovascular device is at least 252.6% of the length of the working segment 120 of the endovascular device. According to some such embodiments, the length of segment 110 of the endovascular device is at least 252.7% of the length of the working segment 120 of the endovascular device. According to some such embodiments, the length of segment 110 of the endovascular device is at least 252.8% of the length of the working segment 120 of the endovascular device. According to some such embodiments, the length of segment 110 of the endovascular device is at least 252.9% of the length of the working segment 120 of the endovascular device. According to some such embodiments, the length of segment 110 of the endovascular device is at least 253% of the length of the working segment 120 of the endovascular device. According to some such embodiments, the length of segment 110 of the endovascular device is at least 253.1% of the length of the working segment 120 of the endovascular device. According to some such embodiments, the length of segment 110 of the endovascular device is at least 253.2% of the length of the working segment 120 of the endovascular device. According to some such embodiments, the length of segment 110 of the endovascular device is at least 253.3% of the length of the working segment 120 of the endovascular device. According to some such embodiments, the length of segment 110 of the endovascular device is at least 253.4% of the length of the working segment 120 of the endovascular device. According to some such embodiments, the length of segment 110 of the endovascular device is at least 253.5% of the length of the working segment 120 of the endovascular device. According to some such embodiments, the length of segment 110 of the endovascular device is at least 253.6% of the length of the working segment 120 of the endovascular device. According to some such embodiments, the length of segment 110 of the endovascular device is at least 253.7% of the length of the working segment 120 of the endovascular device. According to some such embodiments, the length of segment 110 of the endovascular device is at least 253.8% of the length of the working segment 120 of the endovascular device. According to some such embodiments, the length of segment 110 of the endovascular device is at least 253.9% of the length of the working segment 120 of the endovascular device. According to some such embodiments, the length of segment 110 of the endovascular device is at least 254% of the length of the working segment 120 of the endovascular device. According to some such embodiments, the length of segment 110 of the endovascular device is at least 254.1% of the length of the working segment 120 of the endovascular device. According to some such embodiments, the length of segment 110 of the endovascular device is at least 254.2% of the length of the working segment 120 of the endovascular device. According to some such embodiments, the length of segment 110 of the endovascular device is at least 254.3% of the length of the working segment 120 of the endovascular device. According to some such embodiments, the length of segment 110 of the endovascular device is at least 254.4% of the length of the working segment 120 of the endovascular device. According to some such embodiments, the length of segment 110 of the endovascular device is at least 254.5% of the length of the working segment 120 of the endovascular device. According to some such embodiments, the length of segment 110 of the endovascular device is at least 254.6% of the length of the working segment 120 of the endovascular device. According to some such embodiments, the length of segment 110 of the endovascular device is at least 254.7% of the length of the working segment 120 of the endovascular device. According to some such embodiments, the length of segment 110 of the endovascular device is at least 254.8% of the length of the working segment 120 of the endovascular device. According to some such embodiments, the length of segment 110 of the endovascular device is at least 254.9% of the length of the working segment 120 of the endovascular device. According to some such embodiments, the length of segment 110 of the endovascular device is at least 255% of the length of the working segment 120 of the endovascular device. According to some such embodiments, the length of segment 110 of the endovascular device is at least 255.1% of the length of the working segment 120 of the endovascular device. According to some such embodiments, the length of segment 110 of the endovascular device is at least 255.2% of the length of the working segment 120 of the endovascular device. According to some such embodiments, the length of segment 110 of the endovascular device is at least 255.3% of the length of the working segment 120 of the endovascular device. According to some such embodiments, the length of segment 110 of the endovascular device is at least 255.4% of the length of the working segment 120 of the endovascular device. According to some such embodiments, the length of segment 110 of the endovascular device is at least 255.5% of the length of the working segment 120 of the endovascular device. According to some such embodiments, the length of segment 110 of the endovascular device is at least 255.6% of the length of the working segment 120 of the endovascular device. According to some such embodiments, the length of segment 110 of the endovascular device is at least 255.7% of the length of the working segment 120 of the endovascular device. According to some such embodiments, the length of segment 110 of the endovascular device is at least 255.8% of the length of the working segment 120 of the endovascular device. According to some such embodiments, the length of segment 110 of the endovascular device is at least 255.9% of the length of the working segment 120 of the endovascular device.

According to some such embodiments, the length of segment 110 of the endovascular device is at least 256% of the length of the working segment 120 of the endovascular device. According to some such embodiments, the length of segment 110 of the endovascular device is at least 256.1% of the length of the working segment 120 of the endovascular device. According to some such embodiments, the length of segment 110 of the endovascular device is at least 256.2% of the length of the working segment 120 of the endovascular device. According to some such embodiments, the length of segment 110 of the endovascular device is at least 256.3% of the length of the working segment 120 of the endovascular device. According to some such embodiments, the length of segment 110 of the endovascular device is at least 256.4% of the length of the working segment 120 of the endovascular device. According to some such embodiments, the length of segment 110 of the endovascular device is at least 256.5% of the length of the working segment 120 of the endovascular device. According to some such embodiments, the length of segment 110 of the endovascular device is at least 256.6% of the length of the working segment 120 of the endovascular device. According to some such embodiments, the length of segment 110 of the endovascular device is at least 256.7% of the length of the working segment 120 of the endovascular device. According to some such embodiments, the length of segment 110 of the endovascular device is at least 256.8% of the length of the working segment 120 of the endovascular device. According to some such embodiments, the length of segment 110 of the endovascular device is at least 256.9% of the length of the working segment 120 of the endovascular device. According to some such embodiments, the length of segment 110 of the endovascular device is at least 257% of the length of the working segment 120 of the endovascular device.

According to some such embodiments, the length of segment 110 of the endovascular device is at least 257.1% of the length of the working segment 120 of the endovascular device. According to some such embodiments, the length of segment 110 of the endovascular device is at least 257.2% of the length of the working segment 120 of the endovascular device. According to some such embodiments, the length of segment 110 of the endovascular device is at least 257.3% of the length of the working segment 120 of the endovascular device. According to some such embodiments, the length of segment 110 of the endovascular device is at least 257.4% of the length of the working segment 120 of the endovascular device. According to some such embodiments, the length of segment 110 of the endovascular device is at least 257.5% of the length of the working segment 120 of the endovascular device. According to some such embodiments, the length of segment 110 of the endovascular device is at least 257.6% of the length of the working segment 120 of the endovascular device. According to some such embodiments, the length of segment 110 of the endovascular device is at least 257.7% of the length of the working segment 120 of the endovascular device. According to some such embodiments, the length of segment 110 of the endovascular device is at least 257.8% of the length of the working segment 120 of the endovascular device. According to some such embodiments, the length of segment 110 of the endovascular device is at least 257.9% of the length of the working segment 120 of the endovascular device. According to some such embodiments, the length of segment 110 of the endovascular device is at least 258% of the length of the working segment 120 of the endovascular device. According to some such embodiments, the length of segment 110 of the endovascular device is at least 258.1% of the length of the working segment 120 of the endovascular device. According to some such embodiments, the length of segment 110 of the endovascular device is at least 258.2% of the length of the working segment 120 of the endovascular device. According to some such embodiments, the length of segment 110 of the endovascular device is at least 258.3% of the length of the working segment 120 of the endovascular device. According to some such embodiments, the length of segment 110 of the endovascular device is at least 258.4% of the length of the working segment 120 of the endovascular device. According to some such embodiments, the length of segment 110 of the endovascular device is at least 258.5% of the length of the working segment 120 of the endovascular device. According to some such embodiments, the length of segment 110 of the endovascular device is at least 258.6% of the length of the working segment 120 of the endovascular device. According to some such embodiments, the length of segment 110 of the endovascular device is at least 258.7% of the length of the working segment 120 of the endovascular device. According to some such embodiments, the length of segment 110 of the endovascular device is at least 258.8% of the length of the working segment 120 of the endovascular device. According to some such embodiments, the length of segment 110 of the endovascular device is at least 258.9% of the length of the working segment 120 of the endovascular device. According to some such embodiments, the length of segment 110 of the endovascular device is at least 259% of the length of the working segment 120 of the endovascular device. According to some such embodiments, the length of segment 110 of the endovascular device is at least 259.1% of the length of the working segment 120 of the endovascular device. According to some such embodiments, the length of segment 110 of the endovascular device is at least 259.2% of the length of the working segment 120 of the endovascular device. According to some such embodiments, the length of segment 110 of the endovascular device is at least 259.3% of the length of the working segment 120 of the endovascular device. According to some such embodiments, the length of segment 110 of the endovascular device is at least 259.4% of the length of the working segment 120 of the endovascular device. According to some such embodiments, the length of segment 110 of the endovascular device is at least 259.5% of the length of the working segment 120 of the endovascular device. According to some such embodiments, the length of segment 110 of the endovascular device is at least 259.6% of the length of the working segment 120 of the endovascular device. According to some such embodiments, the length of segment 110 of the endovascular device is at least 259.7% of the length of the working segment 120 of the endovascular device. According to some such embodiments, the length of segment 110 of the endovascular device is at least 259.8% of the length of the working segment 120 of the endovascular device. According to some such embodiments, the length of segment 110 of the endovascular device is at least 159.9% of the length of the working segment 120 of the endovascular device.

According to some such embodiments, the length of segment 110 of the endovascular device is at least 260% of the length of the working segment 120 of the endovascular device. According to some such embodiments, the length of segment 110 of the endovascular device is at least 260.1% of the length of the working segment 120 of the endovascular device. According to some such embodiments, the length of segment 110 of the endovascular device is at least 260.2% of the length of the working segment 120 of the endovascular device. According to some such embodiments, the length of segment 110 of the endovascular device is at least 260.3% of the length of the working segment 120 of the endovascular device. According to some such embodiments, the length of segment 110 of the endovascular device is at least 260.4% of the length of the working segment 120 of the endovascular device. According to some such embodiments, the length of segment 110 of the endovascular device is at least 260.5% of the length of the working segment 120 of the endovascular device. According to some such embodiments, the length of segment 110 of the endovascular device is at least 260.6% of the length of the working segment 120 of the endovascular device. According to some such embodiments, the length of segment 110 of the endovascular device is at least 260.7% of the length of the working segment 120 of the endovascular device. According to some such embodiments, the length of segment 110 of the endovascular device is at least 260.8% of the length of the working segment 120 of the endovascular device. According to some such embodiments, the length of segment 110 of the endovascular device is at least 260.9% of the length of the working segment 120 of the endovascular device. According to some such embodiments, the length of segment 110 of the endovascular device is at least 261% of the length of the working segment 120 of the endovascular device. According to some such embodiments, the length of segment 110 of the endovascular device is at least 261.1% of the length of the working segment 120 of the endovascular device. According to some such embodiments, the length of segment 110 of the endovascular device is at least 261.2% of the length of the working segment 120 of the endovascular device. According to some such embodiments, the length of segment 110 of the endovascular device is at least 261.3% of the length of the working segment 120 of the endovascular device. According to some such embodiments, the length of segment 110 of the endovascular device is at least 261.4% of the length of the working segment 120 of the endovascular device. According to some such embodiments, the length of segment 110 of the endovascular device is at least 261.5% of the length of the working segment 120 of the endovascular device. According to some such embodiments, the length of segment 110 of the endovascular device is at least 261.6% of the length of the working segment 120 of the endovascular device. According to some such embodiments, the length of segment 110 of the endovascular device is at least 261.7% of the length of the working segment 120 of the endovascular device. According to some such embodiments, the length of segment 110 of the endovascular device is at least 261.8% of the length of the working segment 120 of the endovascular device. According to some such embodiments, the length of segment 110 of the endovascular device is at least 261.9% of the length of the working segment 120 of the endovascular device. According to some such embodiments, the length of segment 110 of the endovascular device is at least 262% of the length of the working segment 120 of the endovascular device. According to some such embodiments, the length of segment 110 of the endovascular device is at least 262.1% of the length of the working segment 120 of the endovascular device. According to some such embodiments, the length of segment 110 of the endovascular device is at least 262.2% of the length of the working segment 120 of the endovascular device. According to some such embodiments, the length of segment 110 of the endovascular device is at least 262.3% of the length of the working segment 120 of the endovascular device. According to some such embodiments, the length of segment 110 of the endovascular device is at least 262.4% of the length of the working segment 120 of the endovascular device. According to some such embodiments, the length of segment 110 of the endovascular device is at least 262.5% of the length of the working segment 120 of the endovascular device. According to some such embodiments, the length of segment 110 of the endovascular device is at least 262.6% of the length of the working segment 120 of the endovascular device. According to some such embodiments, the length of segment 110 of the endovascular device is at least 262.7% of the length of the working segment 120 of the endovascular device. According to some such embodiments, the length of segment 110 of the endovascular device is at least 262.8% of the length of the working segment 120 of the endovascular device. According to some such embodiments, the length of segment 110 of the endovascular device is at least 262.9% of the length of the working segment 120 of the endovascular device. According to some such embodiments, the length of segment 110 of the endovascular device is at least 263% of the length of the working segment 120 of the endovascular device. According to some such embodiments, the length of segment 110 of the endovascular device is at least 263.1% of the length of the working segment 120 of the endovascular device. According to some such embodiments, the length of segment 110 of the endovascular device is at least 263.2% of the length of the working segment 120 of the endovascular device. According to some such embodiments, the length of segment 110 of the endovascular device is at least 263.3% of the length of the working segment 120 of the endovascular device. According to some such embodiments, the length of segment 110 of the endovascular device is at least 263.4% of the length of the working segment 120 of the endovascular device. According to some such embodiments, the length of segment 110 of the endovascular device is at least 263.5% of the length of the working segment 120 of the endovascular device. According to some such embodiments, the length of segment 110 of the endovascular device is at least 263.6% of the length of the working segment 120 of the endovascular device. According to some such embodiments, the length of segment 110 of the endovascular device is at least 263.7% of the length of the working segment 120 of the endovascular device. According to some such embodiments, the length of segment 110 of the endovascular device is at least 263.8% of the length of the working segment 120 of the endovascular device. According to some such embodiments, the length of segment 110 of the endovascular device is at least 263.9% of the length of the working segment 120 of the endovascular device. According to some such embodiments, the length of segment 110 of the endovascular device is at least 264% of the length of the working segment 120 of the endovascular device. According to some such embodiments, the length of segment 110 of the endovascular device is at least 264.1% of the length of the working segment 120 of the endovascular device. According to some such embodiments, the length of segment 110 of the endovascular device is at least 264.2% of the length of the working segment 120 of the endovascular device. According to some such embodiments, the length of segment 110 of the endovascular device is at least 264.3% of the length of the working segment 120 of the endovascular device. According to some such embodiments, the length of segment 110 of the endovascular device is at least 264.4% of the length of the working segment 120 of the endovascular device. According to some such embodiments, the length of segment 110 of the endovascular device is at least 264.5% of the length of the working segment 120 of the endovascular device. According to some such embodiments, the length of segment 110 of the endovascular device is at least 264.6% of the length of the working segment 120 of the endovascular device. According to some such embodiments, the length of segment 110 of the endovascular device is at least 264.7% of the length of the working segment 120 of the endovascular device. According to some such embodiments, the length of segment 110 of the endovascular device is at least 264.8% of the length of the working segment 120 of the endovascular device. According to some such embodiments, the length of segment 110 of the endovascular device is at least 264.9% of the length of the working segment 120 of the endovascular device. According to some such embodiments, the length of segment 110 of the endovascular device is at least 265% of the length of the working segment 120 of the endovascular device. According to some such embodiments, the length of segment 110 of the endovascular device is at least 265.1% of the length of the working segment 120 of the endovascular device. According to some such embodiments, the length of segment 110 of the endovascular device is at least 265.2% of the length of the working segment 120 of the endovascular device. According to some such embodiments, the length of segment 110 of the endovascular device is at least 265.3% of the length of the working segment 120 of the endovascular device. According to some such embodiments, the length of segment 110 of the endovascular device is at least 265.4% of the length of the working segment 120 of the endovascular device. According to some such embodiments, the length of segment 110 of the endovascular device is at least 265.5% of the length of the working segment 120 of the endovascular device. According to some such embodiments, the length of segment 110 of the endovascular device is at least 265.6% of the length of the working segment 120 of the endovascular device. According to some such embodiments, the length of segment 110 of the endovascular device is at least 265.7% of the length of the working segment 120 of the endovascular device. According to some such embodiments, the length of segment 110 of the endovascular device is at least 265.8% of the length of the working segment 120 of the endovascular device. According to some such embodiments, the length of segment 110 of the endovascular device is at least 265.9% of the length of the working segment 120 of the endovascular device. According to some such embodiments, the length of segment 110 of the endovascular device is at least 266% of the length of the working segment 120 of the endovascular device. According to some such embodiments, the length of segment 110 of the endovascular device is at least 266.1% of the length of the working segment 120 of the endovascular device. According to some such embodiments, the length of segment 110 of the endovascular device is at least 266.2% of the length of the working segment 120 of the endovascular device. According to some such embodiments, the length of segment 110 of the endovascular device is at least 266.3% of the length of the working segment 120 of the endovascular device. According to some such embodiments, the length of segment 110 of the endovascular device is at least 266.4% of the length of the working segment 120 of the endovascular device. According to some such embodiments, the length of segment 110 of the endovascular device is at least 266.5% of the length of the working segment 120 of the endovascular device. According to some such embodiments, the length of segment 110 of the endovascular device is at least 266.6% of the length of the working segment 120 of the endovascular device. According to some such embodiments, the length of segment 110 of the endovascular device is at least 266.7% of the length of the working segment 120 of the endovascular device. According to some such embodiments, the length of segment 110 of the endovascular device is at least 266.8% of the length of the working segment 120 of the endovascular device. According to some such embodiments, the length of segment 110 of the endovascular device is at least 266.9% of the length of the working segment 120 of the endovascular device. According to some such embodiments, the length of segment 110 of the endovascular device is at least 267% of the length of the working segment 120 of the endovascular device. According to some such embodiments, the length of segment 110 of the endovascular device is at least 267.1% of the length of the working segment 120 of the endovascular device. According to some such embodiments, the length of segment 110 of the endovascular device is at least 267.2% of the length of the working segment 120 of the endovascular device. According to some such embodiments, the length of segment 110 of the endovascular device is at least 267.3% of the length of the working segment 120 of the endovascular device. According to some such embodiments, the length of segment 110 of the endovascular device is at least 267.4% of the length of the working segment 120 of the endovascular device. According to some such embodiments, the length of segment 110 of the endovascular device is at least 267.5% of the length of the working segment 120 of the endovascular device. According to some such embodiments, the length of segment 110 of the endovascular device is at least 267.6% of the length of the working segment 120 of the endovascular device. According to some such embodiments, the length of segment 110 of the endovascular device is at least 267.7% of the length of the working segment 120 of the endovascular device. According to some such embodiments, the length of segment 110 of the endovascular device is at least 267.8% of the length of the working segment 120 of the endovascular device. According to some such embodiments, the length of segment 110 of the endovascular device is at least 267.9% of the length of the working segment 120 of the endovascular device. According to some such embodiments, the length of segment 110 of the endovascular device is at least 268% of the length of the working segment 120 of the endovascular device. According to some such embodiments, the length of segment 110 of the endovascular device is at least 268.1% of the length of the working segment 120 of the endovascular device. According to some such embodiments, the length of segment 110 of the endovascular device is at least 268.2% of the length of the working segment 120 of the endovascular device. According to some such embodiments, the length of segment 110 of the endovascular device is at least 268.3% of the length of the working segment 120 of the endovascular device. According to some such embodiments, the length of segment 110 of the endovascular device is at least 268.4% of the length of the working segment 120 of the endovascular device. According to some such embodiments, the length of segment 110 of the endovascular device is at least 268.5% of the length of the working segment 120 of the endovascular device. According to some such embodiments, the length of segment 110 of the endovascular device is at least 268.6% of the length of the working segment 120 of the endovascular device. According to some such embodiments, the length of segment 110 of the endovascular device is at least 268.7% of the length of the working segment 120 of the endovascular device. According to some such embodiments, the length of segment 110 of the endovascular device is at least 268.8% of the length of the working segment 120 of the endovascular device. According to some such embodiments, the length of segment 110 of the endovascular device is at least 268.9% of the length of the working segment 120 of the endovascular device. According to some such embodiments, the length of segment 110 of the endovascular device is at least 269% of the length of the working segment 120 of the endovascular device. According to some such embodiments, the length of segment 110 of the endovascular device is at least 269.1% of the length of the working segment 120 of the endovascular device. According to some such embodiments, the length of segment 110 of the endovascular device is at least 269.2% of the length of the working segment 120 of the endovascular device. According to some such embodiments, the length of segment 110 of the endovascular device is at least 269.3% of the length of the working segment 120 of the endovascular device. According to some such embodiments, the length of segment 110 of the endovascular device is at least 269.4% of the length of the working segment 120 of the endovascular device. According to some such embodiments, the length of segment 110 of the endovascular device is at least 269.5% of the length of the working segment 120 of the endovascular device. According to some such embodiments, the length of segment 110 of the endovascular device is at least 269.6% of the length of the working segment 120 of the endovascular device. According to some such embodiments, the length of segment 110 of the endovascular device is at least 269.7% of the length of the working segment 120 of the endovascular device. According to some such embodiments, the length of segment 110 of the endovascular device is at least 269.8% of the length of the working segment 120 of the endovascular device. According to some such embodiments, the length of segment 110 of the endovascular device is at least 269.9% of the length of the working segment 120 of the endovascular device.

According to some such embodiments, the length of segment 110 of the endovascular device is at least 270% of the length of the working segment 120 of the endovascular device. According to some such embodiments, the length of segment 110 of the endovascular device is at least 270.1% of the length of the working segment 120 of the endovascular device. According to some such embodiments, the length of segment 110 of the endovascular device is at least 270.2% of the length of the working segment 120 of the endovascular device. According to some such embodiments, the length of segment 110 of the endovascular device is at least 270.3% of the length of the working segment 120 of the endovascular device. According to some such embodiments, the length of segment 110 of the endovascular device is at least 270.4% of the length of the working segment 120 of the endovascular device. According to some such embodiments, the length of segment 110 of the endovascular device is at least 270.5% of the length of the working segment 120 of the endovascular device. According to some such embodiments, the length of segment 110 of the endovascular device is at least 270.6% of the length of the working segment 120 of the endovascular device. According to some such embodiments, the length of segment 110 of the endovascular device is at least 270.7% of the length of the working segment 120 of the endovascular device. According to some such embodiments, the length of segment 110 of the endovascular device is at least 270.8% of the length of the working segment 120 of the endovascular device. According to some such embodiments, the length of segment 110 of the endovascular device is at least 270.9% of the length of the working segment 120 of the endovascular device. According to some such embodiments, the length of segment 110 of the endovascular device is at least 271% of the length of the working segment 120 of the endovascular device. According to some such embodiments, the length of segment 110 of the endovascular device is at least 271.1% of the length of the working segment 120 of the endovascular device. According to some such embodiments, the length of segment 110 of the endovascular device is at least 271.2% of the length of the working segment 120 of the endovascular device. According to some such embodiments, the length of segment 110 of the endovascular device is at least 271.3% of the length of the working segment 120 of the endovascular device. According to some such embodiments, the length of segment 110 of the endovascular device is at least 271.4% of the length of the working segment 120 of the endovascular device. According to some such embodiments, the length of segment 110 of the endovascular device is at least 271.5% of the length of the working segment 120 of the endovascular device. According to some such embodiments, the length of segment 110 of the endovascular device is at least 271.6% of the length of the working segment 120 of the endovascular device. According to some such embodiments, the length of segment 110 of the endovascular device is at least 271.7% of the length of the working segment 120 of the endovascular device. According to some such embodiments, the length of segment 110 of the endovascular device is at least 271.8% of the length of the working segment 120 of the endovascular device. According to some such embodiments, the length of segment 110 of the endovascular device is at least 271.9% of the length of the working segment 120 of the endovascular device. According to some such embodiments, the length of segment 110 of the endovascular device is at least 272% of the length of the working segment 120 of the endovascular device. According to some such embodiments, the length of segment 110 of the endovascular device is at least 272.1% of the length of the working segment 120 of the endovascular device. According to some such embodiments, the length of segment 110 of the endovascular device is at least 272.2% of the length of the working segment 120 of the endovascular device. According to some such embodiments, the length of segment 110 of the endovascular device is at least 272.3% of the length of the working segment 120 of the endovascular device. According to some such embodiments, the length of segment 110 of the endovascular device is at least 272.4% of the length of the working segment 120 of the endovascular device. According to some such embodiments, the length of segment 110 of the endovascular device is at least 272.5% of the length of the working segment 120 of the endovascular device. According to some such embodiments, the length of segment 110 of the endovascular device is at least 272.6% of the length of the working segment 120 of the endovascular device. According to some such embodiments, the length of segment 110 of the endovascular device is at least 272.7% of the length of the working segment 120 of the endovascular device. According to some such embodiments, the length of segment 110 of the endovascular device is at least 272.8% of the length of the working segment 120 of the endovascular device. According to some such embodiments, the length of segment 110 of the endovascular device is at least 272.9% of the length of the working segment 120 of the endovascular device. According to some such embodiments, the length of segment 110 of the endovascular device is at least 273% of the length of the working segment 120 of the endovascular device. According to some such embodiments, the length of segment 110 of the endovascular device is at least 273.1% of the length of the working segment 120 of the endovascular device. According to some such embodiments, the length of segment 110 of the endovascular device is at least 273.2% of the length of the working segment 120 of the endovascular device. According to some such embodiments, the length of segment 110 of the endovascular device is at least 273.3% of the length of the working segment 120 of the endovascular device. According to some such embodiments, the length of segment 110 of the endovascular device is at least 273.4% of the length of the working segment 120 of the endovascular device. According to some such embodiments, the length of segment 110 of the endovascular device is at least 273.5% of the length of the working segment 120 of the endovascular device. According to some such embodiments, the length of segment 110 of the endovascular device is at least 273.6% of the length of the working segment 120 of the endovascular device. According to some such embodiments, the length of segment 110 of the endovascular device is at least 273.7% of the length of the working segment 120 of the endovascular device. According to some such embodiments, the length of segment 110 of the endovascular device is at least 273.8% of the length of the working segment 120 of the endovascular device. According to some such embodiments, the length of segment 110 of the endovascular device is at least 273.9% of the length of the working segment 120 of the endovascular device. According to some such embodiments, the length of segment 110 of the endovascular device is at least 274% of the length of the working segment 120 of the endovascular device. According to some such embodiments, the length of segment 110 of the endovascular device is at least 274.1% of the length of the working segment 120 of the endovascular device. According to some such embodiments, the length of segment 110 of the endovascular device is at least 274.2% of the length of the working segment 120 of the endovascular device. According to some such embodiments, the length of segment 110 of the endovascular device is at least 274.3% of the length of the working segment 120 of the endovascular device. According to some such embodiments, the length of segment 110 of the endovascular device is at least 274.4% of the length of the working segment 120 of the endovascular device. According to some such embodiments, the length of segment 110 of the endovascular device is at least 274.5% of the length of the working segment 120 of the endovascular device. According to some such embodiments, the length of segment 110 of the endovascular device is at least 274.6% of the length of the working segment 120 of the endovascular device. According to some such embodiments, the length of segment 110 of the endovascular device is at least 274.7% of the length of the working segment 120 of the endovascular device. According to some such embodiments, the length of segment 110 of the endovascular device is at least 274.8% of the length of the working segment 120 of the endovascular device. According to some such embodiments, the length of segment 110 of the endovascular device is at least 274.9% of the length of the working segment 120 of the endovascular device. According to some such embodiments, the length of segment 110 of the endovascular device is at least 275% of the length of the working segment 120 of the endovascular device.

According to some such embodiments, the length of segment 110 of the endovascular device is at least 275.1% of the length of the working segment 120 of the endovascular device. According to some such embodiments, the length of segment 110 of the endovascular device is at least 275.2% of the length of the working segment 120 of the endovascular device. According to some such embodiments, the length of segment 110 of the endovascular device is at least 275.3% of the length of the working segment 120 of the endovascular device. According to some such embodiments, the length of segment 110 of the endovascular device is at least 275.4% of the length of the working segment 120 of the endovascular device. According to some such embodiments, the length of segment 110 of the endovascular device is at least 275.5% of the length of the working segment 120 of the endovascular device. According to some such embodiments, the length of segment 110 of the endovascular device is at least 275.6% of the length of the working segment 120 of the endovascular device. According to some such embodiments, the length of segment 110 of the endovascular device is at least 275.7% of the length of the working segment 120 of the endovascular device. According to some such embodiments, the length of segment 110 of the endovascular device is at least 275.8% of the length of the working segment 120 of the endovascular device. According to some such embodiments, the length of segment 110 of the endovascular device is at least 275.9% of the length of the working segment 120 of the endovascular device. According to some such embodiments, the length of segment 110 of the endovascular device is at least 276% of the length of the working segment 120 of the endovascular device. According to some such embodiments, the length of segment 110 of the endovascular device is at least 276.1% of the length of the working segment 120 of the endovascular device. According to some such embodiments, the length of segment 110 of the endovascular device is at least 276.2% of the length of the working segment 120 of the endovascular device. According to some such embodiments, the length of segment 110 of the endovascular device is at least 276.3% of the length of the working segment 120 of the endovascular device. According to some such embodiments, the length of segment 110 of the endovascular device is at least 276.4% of the length of the working segment 120 of the endovascular device. According to some such embodiments, the length of segment 110 of the endovascular device is at least 276.5% of the length of the working segment 120 of the endovascular device. According to some such embodiments, the length of segment 110 of the endovascular device is at least 276.6% of the length of the working segment 120 of the endovascular device. According to some such embodiments, the length of segment 110 of the endovascular device is at least 276.7% of the length of the working segment 120 of the endovascular device. According to some such embodiments, the length of segment 110 of the endovascular device is at least 276.8% of the length of the working segment 120 of the endovascular device. According to some such embodiments, the length of segment 110 of the endovascular device is at least 276.9% of the length of the working segment 120 of the endovascular device.

According to some such embodiments, the length of segment 110 of the endovascular device is at least 277.1% of the length of the working segment 120 of the endovascular device. According to some such embodiments, the length of segment 110 of the endovascular device is at least 277.2% of the length of the working segment 120 of the endovascular device. According to some such embodiments, the length of segment 110 of the endovascular device is at least 277.3% of the length of the working segment 120 of the endovascular device. According to some such embodiments, the length of segment 110 of the endovascular device is at least 277.4% of the length of the working segment 120 of the endovascular device. According to some such embodiments, the length of segment 110 of the endovascular device is at least 277.5% of the length of the working segment 120 of the endovascular device. According to some such embodiments, the length of segment 110 of the endovascular device is at least 277.6% of the length of the working segment 120 of the endovascular device. According to some such embodiments, the length of segment 110 of the endovascular device is at least 277.7% of the length of the working segment 120 of the endovascular device. According to some such embodiments, the length of segment 110 of the endovascular device is at least 277.8% of the length of the working segment 120 of the endovascular device. According to some such embodiments, the length of segment 110 of the endovascular device is at least 277.9% of the length of the working segment 120 of the endovascular device. According to some such embodiments, the length of segment 110 of the endovascular device is at least 278% of the length of the working segment 120 of the endovascular device. According to some such embodiments, the length of segment 110 of the endovascular device is at least 278.1% of the length of the working segment 120 of the endovascular device. According to some such embodiments, the length of segment 110 of the endovascular device is at least 278.2% of the length of the working segment 120 of the endovascular device. According to some such embodiments, the length of segment 110 of the endovascular device is at least 278.3% of the length of the working segment 120 of the endovascular device. According to some such embodiments, the length of segment 110 of the endovascular device is at least 278.4% of the length of the working segment 120 of the endovascular device. According to some such embodiments, the length of segment 110 of the endovascular device is at least 278.5% of the length of the working segment 120 of the endovascular device. According to some such embodiments, the length of segment 110 of the endovascular device is at least 278.6% of the length of the working segment 120 of the endovascular device. According to some such embodiments, the length of segment 110 of the endovascular device is at least 278.7% of the length of the working segment 120 of the endovascular device. According to some such embodiments, the length of segment 110 of the endovascular device is at least 278.8% of the length of the working segment 120 of the endovascular device. According to some such embodiments, the length of segment 110 of the endovascular device is at least 278.9% of the length of the working segment 120 of the endovascular device. According to some such embodiments, the length of segment 110 of the endovascular device is at least 279% of the length of the working segment 120 of the endovascular device. According to some such embodiments, the length of segment 110 of the endovascular device is at least 279.1% of the length of the working segment 120 of the endovascular device. According to some such embodiments, the length of segment 110 of the endovascular device is at least 279.2% of the length of the working segment 120 of the endovascular device. According to some such embodiments, the length of segment 110 of the endovascular device is at least 279.3% of the length of the working segment 120 of the endovascular device. According to some such embodiments, the length of segment 110 of the endovascular device is at least 279.4% of the length of the working segment 120 of the endovascular device. According to some such embodiments, the length of segment 110 of the endovascular device is at least 279.5% of the length of the working segment 120 of the endovascular device. According to some such embodiments, the length of segment 110 of the endovascular device is at least 279.6% of the length of the working segment 120 of the endovascular device. According to some such embodiments, the length of segment 110 of the endovascular device is at least 279.7% of the length of the working segment 120 of the endovascular device. According to some such embodiments, the length of segment 110 of the endovascular device is at least 279.8% of the length of the working segment 120 of the endovascular device. According to some such embodiments, the length of segment 110 of the endovascular device is at least 279.9% of the length of the working segment 120 of the endovascular device. According to some such embodiments, the length of segment 110 of the endovascular device is at least 280% of the length of the working segment 120 of the endovascular device. According to some such embodiments, the length of segment 110 of the endovascular device is at least 280.1% of the length of the working segment 120 of the endovascular device. According to some such embodiments, the length of segment 110 of the endovascular device is at least 280.2% of the length of the working segment 120 of the endovascular device. According to some such embodiments, the length of segment 110 of the endovascular device is at least 280.3% of the length of the working segment 120 of the endovascular device. According to some such embodiments, the length of segment 110 of the endovascular device is at least 280.4% of the length of the working segment 120 of the endovascular device. According to some such embodiments, the length of segment 110 of the endovascular device is at least 280.5% of the length of the working segment 120 of the endovascular device. According to some such embodiments, the length of segment 110 of the endovascular device is at least 280.6% of the length of the working segment 120 of the endovascular device. According to some such embodiments, the length of segment 110 of the endovascular device is at least 280.7% of the length of the working segment 120 of the endovascular device. According to some such embodiments, the length of segment 110 of the endovascular device is at least 280.8% of the length of the working segment 120 of the endovascular device. According to some such embodiments, the length of segment 110 of the endovascular device is at least 280.9% of the length of the working segment 120 of the endovascular device.

According to some such embodiments, the length of segment 110 of the endovascular device is at least 281% of the length of the working segment 120 of the endovascular device. According to some such embodiments, the length of segment 110 of the endovascular device is at least 281.1% of the length of the working segment 120 of the endovascular device. According to some such embodiments, the length of segment 110 of the endovascular device is at least 281.2% of the length of the working segment 120 of the endovascular device. According to some such embodiments, the length of segment 110 of the endovascular device is at least 281.3% of the length of the working segment 120 of the endovascular device. According to some such embodiments, the length of segment 110 of the endovascular device is at least 281.4% of the length of the working segment 120 of the endovascular device. According to some such embodiments, the length of segment 110 of the endovascular device is at least 281.5% of the length of the working segment 120 of the endovascular device. According to some such embodiments, the length of segment 110 of the endovascular device is at least 281.6% of the length of the working segment 120 of the endovascular device. According to some such embodiments, the length of segment 110 of the endovascular device is at least 281.7% of the length of the working segment 120 of the endovascular device. According to some such embodiments, the length of segment 110 of the endovascular device is at least 281.8% of the length of the working segment 120 of the endovascular device. According to some such embodiments, the length of segment 110 of the endovascular device is at least 281.9% of the length of the working segment 120 of the endovascular device. According to some such embodiments, the length of segment 110 of the endovascular device is at least 282% of the length of the working segment 120 of the endovascular device. According to some such embodiments, the length of segment 110 of the endovascular device is at least 282.1% of the length of the working segment 120 of the endovascular device. According to some such embodiments, the length of segment 110 of the endovascular device is at least 282.2% of the length of the working segment 120 of the endovascular device. According to some such embodiments, the length of segment 110 of the endovascular device is at least 282.3% of the length of the working segment 120 of the endovascular device. According to some such embodiments, the length of segment 110 of the endovascular device is at least 282.4% of the length of the working segment 120 of the endovascular device. According to some such embodiments, the length of segment 110 of the endovascular device is at least 282.5% of the length of the working segment 120 of the endovascular device. According to some such embodiments, the length of segment 110 of the endovascular device is at least 282.6% of the length of the working segment 120 of the endovascular device. According to some such embodiments, the length of segment 110 of the endovascular device is at least 282.7% of the length of the working segment 120 of the endovascular device. According to some such embodiments, the length of segment 110 of the endovascular device is at least 282.8% of the length of the working segment 120 of the endovascular device. According to some such embodiments, the length of segment 110 of the endovascular device is at least 282.9% of the length of the working segment 120 of the endovascular device. According to some such embodiments, the length of segment 110 of the endovascular device is at least 283% of the length of the working segment 120 of the endovascular device. According to some such embodiments, the length of segment 110 of the endovascular device is at least 283.1% of the length of the working segment 120 of the endovascular device. According to some such embodiments, the length of segment 110 of the endovascular device is at least 283.2% of the length of the working segment 120 of the endovascular device. According to some such embodiments, the length of segment 110 of the endovascular device is at least 283.3% of the length of the working segment 120 of the endovascular device. According to some such embodiments, the length of segment 110 of the endovascular device is at least 283.4% of the length of the working segment 120 of the endovascular device. According to some such embodiments, the length of segment 110 of the endovascular device is at least 283.5% of the length of the working segment 120 of the endovascular device. According to some such embodiments, the length of segment 110 of the endovascular device is at least 283.6% of the length of the working segment 120 of the endovascular device. According to some such embodiments, the length of segment 110 of the endovascular device is at least 283.7% of the length of the working segment 120 of the endovascular device. According to some such embodiments, the length of segment 110 of the endovascular device is at least 283.8% of the length of the working segment 120 of the endovascular device. According to some such embodiments, the length of segment 110 of the endovascular device is at least 283.9% of the length of the working segment 120 of the endovascular device. According to some such embodiments, the length of segment 110 of the endovascular device is at least 284% of the length of the working segment 120 of the endovascular device. According to some such embodiments, the length of segment 110 of the endovascular device is at least 284.1% of the length of the working segment 120 of the endovascular device. According to some such embodiments, the length of segment 110 of the endovascular device is at least 284.2% of the length of the working segment 120 of the endovascular device. According to some such embodiments, the length of segment 110 of the endovascular device is at least 284.3% of the length of the working segment 120 of the endovascular device. According to some such embodiments, the length of segment 110 of the endovascular device is at least 284.4% of the length of the working segment 120 of the endovascular device. According to some such embodiments, the length of segment 110 of the endovascular device is at least 284.5% of the length of the working segment 120 of the endovascular device. According to some such embodiments, the length of segment 110 of the endovascular device is at least 284.6% of the length of the working segment 120 of the endovascular device. According to some such embodiments, the length of segment 110 of the endovascular device is at least 284.7% of the length of the working segment 120 of the endovascular device. According to some such embodiments, the length of segment 110 of the endovascular device is at least 284.8% of the length of the working segment 120 of the endovascular device. According to some such embodiments, the length of segment 110 of the endovascular device is at least 284.9% of the length of the working segment 120 of the endovascular device. According to some such embodiments, the length of segment 110 of the endovascular device is at least 285% of the length of the working segment 120 of the endovascular device. According to some such embodiments, the length of segment 110 of the endovascular device is at least 285.1% of the length of the working segment 120 of the endovascular device. According to some such embodiments, the length of segment 110 of the endovascular device is at least 285.2% of the length of the working segment 120 of the endovascular device. According to some such embodiments, the length of segment 110 of the endovascular device is at least 285.3% of the length of the working segment 120 of the endovascular device. According to some such embodiments, the length of segment 110 of the endovascular device is at least 285.4% of the length of the working segment 120 of the endovascular device. According to some such embodiments, the length of segment 110 of the endovascular device is at least 285.5% of the length of the working segment 120 of the endovascular device. According to some such embodiments, the length of segment 110 of the endovascular device is at least 285.6% of the length of the working segment 120 of the endovascular device. According to some such embodiments, the length of segment 110 of the endovascular device is at least 285.7% of the length of the working segment 120 of the endovascular device. According to some such embodiments, the length of segment 110 of the endovascular device is at least 285.8% of the length of the working segment 120 of the endovascular device. According to some such embodiments, the length of segment 110 of the endovascular device is at least 285.9% of the length of the working segment 120 of the endovascular device. According to some such embodiments, the length of segment 110 of the endovascular device is at least 286% of the length of the working segment 120 of the endovascular device. According to some such embodiments, the length of segment 110 of the endovascular device is at least 286.1% of the length of the working segment 120 of the endovascular device. According to some such embodiments, the length of segment 110 of the endovascular device is at least 286.2% of the length of the working segment 120 of the endovascular device. According to some such embodiments, the length of segment 110 of the endovascular device is at least 286.3% of the length of the working segment 120 of the endovascular device. According to some such embodiments, the length of segment 110 of the endovascular device is at least 286.4% of the length of the working segment 120 of the endovascular device. According to some such embodiments, the length of segment 110 of the endovascular device is at least 286.5% of the length of the working segment 120 of the endovascular device. According to some such embodiments, the length of segment 110 of the endovascular device is at least 286.6% of the length of the working segment 120 of the endovascular device. According to some such embodiments, the length of segment 110 of the endovascular device is at least 286.7% of the length of the working segment 120 of the endovascular device. According to some such embodiments, the length of segment 110 of the endovascular device is at least 286.8% of the length of the working segment 120 of the endovascular device. According to some such embodiments, the length of segment 110 of the endovascular device is at least 286.9% of the length of the working segment 120 of the endovascular device. According to some such embodiments, the length of segment 110 of the endovascular device is at least 287% of the length of the working segment 120 of the endovascular device. According to some such embodiments, the length of segment 110 of the endovascular device is at least 287.1% of the length of the working segment 120 of the endovascular device. According to some such embodiments, the length of segment 110 of the endovascular device is at least 287.2% of the length of the working segment 120 of the endovascular device. According to some such embodiments, the length of segment 110 of the endovascular device is at least 287.3% of the length of the working segment 120 of the endovascular device. According to some such embodiments, the length of segment 110 of the endovascular device is at least 287.4% of the length of the working segment 120 of the endovascular device. According to some such embodiments, the length of segment 110 of the endovascular device is at least 287.5% of the length of the working segment 120 of the endovascular device. According to some such embodiments, the length of segment 110 of the endovascular device is at least 287.6% of the length of the working segment 120 of the endovascular device. According to some such embodiments, the length of segment 110 of the endovascular device is at least 287.7% of the length of the working segment 120 of the endovascular device. According to some such embodiments, the length of segment 110 of the endovascular device is at least 287.8% of the length of the working segment 120 of the endovascular device. According to some such embodiments, the length of segment 110 of the endovascular device is at least 287.9% of the length of the working segment 120 of the endovascular device. According to some such embodiments, the length of segment 110 of the endovascular device is at least 288% of the length of the working segment 120 of the endovascular device. According to some such embodiments, the length of segment 110 of the endovascular device is at least 288.1% of the length of the working segment 120 of the endovascular device. According to some such embodiments, the length of segment 110 of the endovascular device is at least 288.2% of the length of the working segment 120 of the endovascular device. According to some such embodiments, the length of segment 110 of the endovascular device is at least 288.3% of the length of the working segment 120 of the endovascular device. According to some such embodiments, the length of segment 110 of the endovascular device is at least 288.4% of the length of the working segment 120 of the endovascular device. According to some such embodiments, the length of segment 110 of the endovascular device is at least 288.5% of the length of the working segment 120 of the endovascular device. According to some such embodiments, the length of segment 110 of the endovascular device is at least 288.6% of the length of the working segment 120 of the endovascular device. According to some such embodiments, the length of segment 110 of the endovascular device is at least 288.7% of the length of the working segment 120 of the endovascular device. According to some such embodiments, the length of segment 110 of the endovascular device is at least 288.8% of the length of the working segment 120 of the endovascular device. According to some such embodiments, the length of segment 110 of the endovascular device is at least 288.9% of the length of the working segment 120 of the endovascular device. According to some such embodiments, the length of segment 110 of the endovascular device is at least 289% of the length of the working segment 120 of the endovascular device. According to some such embodiments, the length of segment 110 of the endovascular device is at least 289.1% of the length of the working segment 120 of the endovascular device. According to some such embodiments, the length of segment 110 of the endovascular device is at least 289.2% of the length of the working segment 120 of the endovascular device. According to some such embodiments, the length of segment 110 of the endovascular device is at least 289.3% of the length of the working segment 120 of the endovascular device. According to some such embodiments, the length of segment 110 of the endovascular device is at least 289.4% of the length of the working segment 120 of the endovascular device. According to some such embodiments, the length of segment 110 of the endovascular device is at least 289.5% of the length of the working segment 120 of the endovascular device. According to some such embodiments, the length of segment 110 of the endovascular device is at least 289.6% of the length of the working segment 120 of the endovascular device. According to some such embodiments, the length of segment 110 of the endovascular device is at least 289.7% of the length of the working segment 120 of the endovascular device. According to some such embodiments, the length of segment 110 of the endovascular device is at least 289.8% of the length of the working segment 120 of the endovascular device. According to some such embodiments, the length of segment 110 of the endovascular device is at least 289.9% of the length of the working segment 120 of the endovascular device.

According to some such embodiments, the length of segment 110 of the endovascular device is at least 290% of the length of the working segment 120 of the endovascular device. According to some such embodiments, the length of segment 110 of the endovascular device is at least 290.1% of the length of the working segment 120 of the endovascular device. According to some such embodiments, the length of segment 110 of the endovascular device is at least 290.2% of the length of the working segment 120 of the endovascular device. According to some such embodiments, the length of segment 110 of the endovascular device is at least 290.3% of the length of the working segment 120 of the endovascular device. According to some such embodiments, the length of segment 110 of the endovascular device is at least 290.4% of the length of the working segment 120 of the endovascular device. According to some such embodiments, the length of segment 110 of the endovascular device is at least 290.5% of the length of the working segment 120 of the endovascular device. According to some such embodiments, the length of segment 110 of the endovascular device is at least 290.6% of the length of the working segment 120 of the endovascular device. According to some such embodiments, the length of segment 110 of the endovascular device is at least 290.7% of the length of the working segment 120 of the endovascular device. According to some such embodiments, the length of segment 110 of the endovascular device is at least 290.8% of the length of the working segment 120 of the endovascular device. According to some such embodiments, the length of segment 110 of the endovascular device is at least 290.9% of the length of the working segment 120 of the endovascular device. According to some such embodiments, the length of segment 110 of the endovascular device is at least 291% of the length of the working segment 120 of the endovascular device. According to some such embodiments, the length of segment 110 of the endovascular device is at least 291.1% of the length of the working segment 120 of the endovascular device. According to some such embodiments, the length of segment 110 of the endovascular device is at least 291.2% of the length of the working segment 120 of the endovascular device. According to some such embodiments, the length of segment 110 of the endovascular device is at least 291.3% of the length of the working segment 120 of the endovascular device. According to some such embodiments, the length of segment 110 of the endovascular device is at least 291.4% of the length of the working segment 120 of the endovascular device. According to some such embodiments, the length of segment 110 of the endovascular device is at least 291.5% of the length of the working segment 120 of the endovascular device. According to some such embodiments, the length of segment 110 of the endovascular device is at least 291.6% of the length of the working segment 120 of the endovascular device. According to some such embodiments, the length of segment 110 of the endovascular device is at least 291.7% of the length of the working segment 120 of the endovascular device. According to some such embodiments, the length of segment 110 of the endovascular device is at least 291.8% of the length of the working segment 120 of the endovascular device. According to some such embodiments, the length of segment 110 of the endovascular device is at least 291.9% of the length of the working segment 120 of the endovascular device. According to some such embodiments, the length of segment 110 of the endovascular device is at least 292% of the length of the working segment 120 of the endovascular device. According to some such embodiments, the length of segment 110 of the endovascular device is at least 292.1% of the length of the working segment 120 of the endovascular device. According to some such embodiments, the length of segment 110 of the endovascular device is at least 292.2% of the length of the working segment 120 of the endovascular device. According to some such embodiments, the length of segment 110 of the endovascular device is at least 292.3% of the length of the working segment 120 of the endovascular device. According to some such embodiments, the length of segment 110 of the endovascular device is at least 292.4% of the length of the working segment 120 of the endovascular device. According to some such embodiments, the length of segment 110 of the endovascular device is at least 292.5% of the length of the working segment 120 of the endovascular device. According to some such embodiments, the length of segment 110 of the endovascular device is at least 292.6% of the length of the working segment 120 of the endovascular device. According to some such embodiments, the length of segment 110 of the endovascular device is at least 292.7% of the length of the working segment 120 of the endovascular device. According to some such embodiments, the length of segment 110 of the endovascular device is at least 292.8% of the length of the working segment 120 of the endovascular device. According to some such embodiments, the length of segment 110 of the endovascular device is at least 292.9% of the length of the working segment 120 of the endovascular device. According to some such embodiments, the length of segment 110 of the endovascular device is at least 293% of the length of the working segment 120 of the endovascular device. According to some such embodiments, the length of segment 110 of the endovascular device is at least 293.1% of the length of the working segment 120 of the endovascular device. According to some such embodiments, the length of segment 110 of the endovascular device is at least 293.2% of the length of the working segment 120 of the endovascular device. According to some such embodiments, the length of segment 110 of the endovascular device is at least 293.3% of the length of the working segment 120 of the endovascular device. According to some such embodiments, the length of segment 110 of the endovascular device is at least 293.4% of the length of the working segment 120 of the endovascular device. According to some such embodiments, the length of segment 110 of the endovascular device is at least 293.5% of the length of the working segment 120 of the endovascular device. According to some such embodiments, the length of segment 110 of the endovascular device is at least 293.6% of the length of the working segment 120 of the endovascular device. According to some such embodiments, the length of segment 110 of the endovascular device is at least 293.7% of the length of the working segment 120 of the endovascular device. According to some such embodiments, the length of segment 110 of the endovascular device is at least 293.8% of the length of the working segment 120 of the endovascular device. According to some such embodiments, the length of segment 110 of the endovascular device is at least 293.9% of the length of the working segment 120 of the endovascular device. According to some such embodiments, the length of segment 110 of the endovascular device is at least 294% of the length of the working segment 120 of the endovascular device. According to some such embodiments, the length of segment 110 of the endovascular device is at least 294.1% of the length of the working segment 120 of the endovascular device. According to some such embodiments, the length of segment 110 of the endovascular device is at least 294.2% of the length of the working segment 120 of the endovascular device. According to some such embodiments, the length of segment 110 of the endovascular device is at least 294.3% of the length of the working segment 120 of the endovascular device. According to some such embodiments, the length of segment 110 of the endovascular device is at least 294.4% of the length of the working segment 120 of the endovascular device. According to some such embodiments, the length of segment 110 of the endovascular device is at least 294.5% of the length of the working segment 120 of the endovascular device. According to some such embodiments, the length of segment 110 of the endovascular device is at least 294.6% of the length of the working segment 120 of the endovascular device. According to some such embodiments, the length of segment 110 of the endovascular device is at least 294.7% of the length of the working segment 120 of the endovascular device. According to some such embodiments, the length of segment 110 of the endovascular device is at least 294.8% of the length of the working segment 120 of the endovascular device. According to some such embodiments, the length of segment 110 of the endovascular device is at least 294.9% of the length of the working segment 120 of the endovascular device. According to some such embodiments, the length of segment 110 of the endovascular device is at least 295% of the length of the working segment 120 of the endovascular device. According to some such embodiments, the length of segment 110 of the endovascular device is at least 295.1% of the length of the working segment 120 of the endovascular device. According to some such embodiments, the length of segment 110 of the endovascular device is at least 295.2% of the length of the working segment 120 of the endovascular device. According to some such embodiments, the length of segment 110 of the endovascular device is at least 295.3% of the length of the working segment 120 of the endovascular device. According to some such embodiments, the length of segment 110 of the endovascular device is at least 295.4% of the length of the working segment 120 of the endovascular device. According to some such embodiments, the length of segment 110 of the endovascular device is at least 295.5% of the length of the working segment 120 of the endovascular device. According to some such embodiments, the length of segment 110 of the endovascular device is at least 295.6% of the length of the working segment 120 of the endovascular device. According to some such embodiments, the length of segment 110 of the endovascular device is at least 295.7% of the length of the working segment 120 of the endovascular device. According to some such embodiments, the length of segment 110 of the endovascular device is at least 295.8% of the length of the working segment 120 of the endovascular device. According to some such embodiments, the length of segment 110 of the endovascular device is at least 295.9% of the length of the working segment 120 of the endovascular device. According to some such embodiments, the length of segment 110 of the endovascular device is at least 296% of the length of the working segment 120 of the endovascular device. According to some such embodiments, the length of segment 110 of the endovascular device is at least 296.1% of the length of the working segment 120 of the endovascular device. According to some such embodiments, the length of segment 110 of the endovascular device is at least 296.2% of the length of the working segment 120 of the endovascular device. According to some such embodiments, the length of segment 110 of the endovascular device is at least 296.3% of the length of the working segment 120 of the endovascular device. According to some such embodiments, the length of segment 110 of the endovascular device is at least 296.4% of the length of the working segment 120 of the endovascular device. According to some such embodiments, the length of segment 110 of the endovascular device is at least 296.5% of the length of the working segment 120 of the endovascular device. According to some such embodiments, the length of segment 110 of the endovascular device is at least 296.6% of the length of the working segment 120 of the endovascular device. According to some such embodiments, the length of segment 110 of the endovascular device is at least 296.7% of the length of the working segment 120 of the endovascular device. According to some such embodiments, the length of segment 110 of the endovascular device is at least 296.8% of the length of the working segment 120 of the endovascular device. According to some such embodiments, the length of segment 110 of the endovascular device is at least 296.9% of the length of the working segment 120 of the endovascular device. According to some such embodiments, the length of segment 110 of the endovascular device is at least 297% of the length of the working segment 120 of the endovascular device. According to some such embodiments, the length of segment 110 of the endovascular device is at least 297.1% of the length of the working segment 120 of the endovascular device. According to some such embodiments, the length of segment 110 of the endovascular device is at least 297.2% of the length of the working segment 120 of the endovascular device. According to some such embodiments, the length of segment 110 of the endovascular device is at least 297.3% of the length of the working segment 120 of the endovascular device. According to some such embodiments, the length of segment 110 of the endovascular device is at least 297.4% of the length of the working segment 120 of the endovascular device. According to some such embodiments, the length of segment 110 of the endovascular device is at least 297.5% of the length of the working segment 120 of the endovascular device. According to some such embodiments, the length of segment 110 of the endovascular device is at least 297.6% of the length of the working segment 120 of the endovascular device. According to some such embodiments, the length of segment 110 of the endovascular device is at least 297.7% of the length of the working segment 120 of the endovascular device. According to some such embodiments, the length of segment 110 of the endovascular device is at least 297.8% of the length of the working segment 120 of the endovascular device. According to some such embodiments, the length of segment 110 of the endovascular device is at least 297.9% of the length of the working segment 120 of the endovascular device. According to some such embodiments, the length of segment 110 of the endovascular device is at least 298% of the length of the working segment 120 of the endovascular device. According to some such embodiments, the length of segment 110 of the endovascular device is at least 298.1% of the length of the working segment 120 of the endovascular device. According to some such embodiments, the length of segment 110 of the endovascular device is at least 298.2% of the length of the working segment 120 of the endovascular device. According to some such embodiments, the length of segment 110 of the endovascular device is at least 298.3% of the length of the working segment 120 of the endovascular device. According to some such embodiments, the length of segment 110 of the endovascular device is at least 298.4% of the length of the working segment 120 of the endovascular device. According to some such embodiments, the length of segment 110 of the endovascular device is at least 298.5% of the length of the working segment 120 of the endovascular device. According to some such embodiments, the length of segment 110 of the endovascular device is at least 298.6% of the length of the working segment 120 of the endovascular device. According to some such embodiments, the length of segment 110 of the endovascular device is at least 298.7% of the length of the working segment 120 of the endovascular device. According to some such embodiments, the length of segment 110 of the endovascular device is at least 298.8% of the length of the working segment 120 of the endovascular device. According to some such embodiments, the length of segment 110 of the endovascular device is at least 298.9% of the length of the working segment 120 of the endovascular device. According to some such embodiments, the length of segment 110 of the endovascular device is at least 299% of the length of the working segment 120 of the endovascular device. According to some such embodiments, the length of segment 110 of the endovascular device is at least 299.1% of the length of the working segment 120 of the endovascular device. According to some such embodiments, the length of segment 110 of the endovascular device is at least 299.2% of the length of the working segment 120 of the endovascular device. According to some such embodiments, the length of segment 110 of the endovascular device is at least 299.3% of the length of the working segment 120 of the endovascular device. According to some such embodiments, the length of segment 110 of the endovascular device is at least 299.4% of the length of the working segment 120 of the endovascular device. According to some such embodiments, the length of segment 110 of the endovascular device is at least 299.5% of the length of the working segment 120 of the endovascular device. According to some such embodiments, the length of segment 110 of the endovascular device is at least 299.6% of the length of the working segment 120 of the endovascular device. According to some such embodiments, the length of segment 110 of the endovascular device is at least 299.7% of the length of the working segment 120 of the endovascular device. According to some such embodiments, the length of segment 110 of the endovascular device is at least 299.8% of the length of the working segment 120 of the endovascular device. According to some such embodiments, the length of segment 110 of the endovascular device is at least 299.9% of the length of the working segment 120 of the endovascular device. According to some such embodiments, the length of segment 110 of the endovascular device is about 300% of the length of the working segment 120 of the endovascular device.

According to some embodiments, the length of segment 110 of the endovascular device is at least 20.1 cm. According to some embodiments, the length of segment 110 of the endovascular device is at least 20.2 cm. According to some embodiments, the length of segment 110 of the endovascular device is at least 20.3 cm. According to some embodiments, the length of segment 110 of the endovascular device is at least 20.4 cm. According to some embodiments, the length of segment 110 of the endovascular device is at least 20.5 cm. According to some embodiments, the length of segment 110 of the endovascular device is at least 20.6 cm. According to some embodiments, the length of segment 110 of the endovascular device is at least 20.7 cm. According to some embodiments, the length of segment 110 of the endovascular device is at least 20.8 cm. According to some embodiments, the length of segment 110 of the endovascular device is at least 20.9 cm. According to some embodiments, the length of segment 110 of the endovascular device is at least 21 cm. According to some embodiments, the length of segment 110 of the endovascular device is at least 21.1 cm. According to some embodiments, the length of segment 110 of the endovascular device is at least 21.2 cm. According to some embodiments, the length of segment 110 of the endovascular device is at least 21.3 cm. According to some embodiments, the length of segment 110 of the endovascular device is at least 21.4 cm. According to some embodiments, the length of segment 110 of the endovascular device is at least 21.5 cm. According to some embodiments, the length of segment 110 of the endovascular device is at least 21.6 cm. According to some embodiments, the length of segment 110 of the endovascular device is at least 21.7 cm. According to some embodiments, the length of segment 110 of the endovascular device is at least 21.8 cm. According to some embodiments, the length of segment 110 of the endovascular device is at least 21.9 cm. According to some embodiments, the length of segment 110 of the endovascular device is at least 30 cm. According to some embodiments, the length of segment 110 of the endovascular device is at least 30.1 cm. According to some embodiments, the length of segment 110 of the endovascular device is at least 30.2 cm. According to some embodiments, the length of segment 110 of the endovascular device is at least 30.3 cm. According to some embodiments, the length of segment 110 of the endovascular device is at least 30.4 cm. According to some embodiments, the length of segment 110 of the endovascular device is at least 30.5 cm. According to some embodiments, the length of segment 110 of the endovascular device is at least 30.6 cm. According to some embodiments, the length of segment 110 of the endovascular device is at least 30.7 cm. According to some embodiments, the length of segment 110 of the endovascular device is at least 30.8 cm. According to some embodiments, the length of segment 110 of the endovascular device is at least 30.9 cm. According to some embodiments, the length of segment 110 of the endovascular device is at least 31.0 cm. According to some embodiments, the length of segment 110 of the endovascular device is at least 31.1 cm. According to some embodiments, the length of segment 110 of the endovascular device is at least 31.2 cm. According to some embodiments, the length of segment 110 of the endovascular device is at least 31.3 cm. According to some embodiments, the length of segment 110 of the endovascular device is at least 31.4 cm. According to some embodiments, the length of segment 110 of the endovascular device is at least 31.5 cm. According to some embodiments, the length of segment 110 of the endovascular device is at least 31.6 cm. According to some embodiments, the length of segment 110 of the endovascular device is at least 31.7 cm. According to some embodiments, the length of segment 110 of the endovascular device is at least 31.8 cm. According to some embodiments, the length of segment 110 of the endovascular device is at least 31.9 cm. According to some embodiments, the length of segment 110 of the endovascular device is at least 32 cm. According to some embodiments, the length of segment 110 of the endovascular device is at least 32.1 cm. According to some embodiments, the length of segment 110 of the endovascular device is at least 32.2 cm. According to some embodiments, the length of segment 110 of the endovascular device is at least 32.3 cm. According to some embodiments, the length of segment 110 of the endovascular device is at least 32.4 cm. According to some embodiments, the length of segment 110 of the endovascular device is at least 32.5 cm. According to some embodiments, the length of segment 110 of the endovascular device is at least 32.6 cm. According to some embodiments, the length of segment 110 of the endovascular device is at least 32.7 cm. According to some embodiments, the length of segment 110 of the endovascular device is at least 32.8 cm. According to some embodiments, the length of segment 110 of the endovascular device is at least 32.9 cm.

According to some embodiments, the length of segment 110 of the endovascular device is at least 33 cm. According to some embodiments, the length of segment 110 of the endovascular device is at least 33.1 cm. According to some embodiments, the length of segment 110 of the endovascular device is at least 33.2 cm. According to some embodiments, the length of segment 110 of the endovascular device is at least 33.3 cm. According to some embodiments, the length of segment 110 of the endovascular device is at least 33.4 cm. According to some embodiments, the length of segment 110 of the endovascular device is at least 33.5 cm. According to some embodiments, the length of segment 110 of the endovascular device is at least 33.6 cm. According to some embodiments, the length of segment 110 of the endovascular device is at least 33.7 cm. According to some embodiments, the length of segment 110 of the endovascular device is at least 33.8 cm. According to some embodiments, the length of segment 110 of the endovascular device is at least 33.9 cm.

According to some embodiments, the length of segment 110 of the endovascular device is at least 34 cm. According to some embodiments, the length of segment 110 of the endovascular device is at least 34.1 cm. According to some embodiments, the length of segment 110 of the endovascular device is at least 34.2 cm. According to some embodiments, the length of segment 110 of the endovascular device is at least 34.3 cm. According to some embodiments, the length of segment 110 of the endovascular device is at least 34.4 cm. According to some embodiments, the length of segment 110 of the endovascular device is at least 34.5 cm. According to some embodiments, the length of segment 110 of the endovascular device is at least 34.6 cm. According to some embodiments, the length of segment 110 of the endovascular device is at least 34.7 cm. According to some embodiments, the length of segment 110 of the endovascular device is at least 34.8 cm. According to some embodiments, the length of segment 110 of the endovascular device is at least 34.9 cm.

According to some embodiments, the length of segment 110 of the endovascular device is at least 35 cm. According to some embodiments, the length of segment 110 of the endovascular device is at least 35.1 cm. According to some embodiments, the length of segment 110 of the endovascular device is at least 35.2 cm. According to some embodiments, the length of segment 110 of the endovascular device is at least 35.3 cm. According to some embodiments, the length of segment 110 of the endovascular device is at least 35.4 cm. According to some embodiments, the length of segment 110 of the endovascular device is at least 35.5 cm. According to some embodiments, the length of segment 110 of the endovascular device is at least 35.6 cm. According to some embodiments, the length of segment 110 of the endovascular device is at least 35.7 cm. According to some embodiments, the length of segment 110 of the endovascular device is at least 35.8 cm. According to some embodiments, the length of segment 110 of the endovascular device is at least 35.9 cm.

According to some embodiments, the length of segment 110 of the endovascular device is at least 36 cm. According to some embodiments, the length of segment 110 of the endovascular device is at least 36.1 cm. According to some embodiments, the length of segment 110 of the endovascular device is at least 36.2 cm. According to some embodiments, the length of segment 110 of the endovascular device is at least 36.3 cm. According to some embodiments, the length of segment 110 of the endovascular device is at least 36.4 cm. According to some embodiments, the length of segment 110 of the endovascular device is at least 36.5 cm. According to some embodiments, the length of segment 110 of the endovascular device is at least 36.6 cm. According to some embodiments, the length of segment 110 of the endovascular device is at least 36.7 cm. According to some embodiments, the length of segment 110 of the endovascular device is at least 36.8 cm. According to some embodiments, the length of segment 110 of the endovascular device is at least 36.9 cm.

According to some embodiments, the length of segment 110 of the endovascular device is at least 37 cm. According to some embodiments, the length of segment 110 of the endovascular device is at least 37.1 cm. According to some embodiments, the length of segment 110 of the endovascular device is at least 37.2 cm. According to some embodiments, the length of segment 110 of the endovascular device is at least 37.3 cm. According to some embodiments, the length of segment 110 of the endovascular device is at least 37.4 cm. According to some embodiments, the length of segment 110 of the endovascular device is at least 37.5 cm. According to some embodiments, the length of segment 110 of the endovascular device is at least 37.6 cm. According to some embodiments, the length of segment 110 of the endovascular device is at least 37.7 cm. According to some embodiments, the length of segment 110 of the endovascular device is at least 37.8 cm. According to some embodiments, the length of segment 110 of the endovascular device is at least 37.9 cm.

According to some embodiments, the length of segment 110 of the endovascular device is at least 38 cm. According to some embodiments, the length of segment 110 of the endovascular device is at least 38.1 cm. According to some embodiments, the length of segment 110 of the endovascular device is at least 38.2 cm. According to some embodiments, the length of segment 110 of the endovascular device is at least 38.3 cm. According to some embodiments, the length of segment 110 of the endovascular device is at least 38.4 cm. According to some embodiments, the length of segment 110 of the endovascular device is at least 38.5 cm. According to some embodiments, the length of segment 110 of the endovascular device is at least 38.6 cm. According to some embodiments, the length of segment 110 of the endovascular device is at least 38.7 cm. According to some embodiments, the length of segment 110 of the endovascular device is at least 38.8 cm. According to some embodiments, the length of segment 110 of the endovascular device is at least 38.9 cm.

According to some embodiments, the length of segment 110 of the endovascular device is at least 39 cm. According to some embodiments, the length of segment 110 of the endovascular device is at least 39.1 cm. According to some embodiments, the length of segment 110 of the endovascular device is at least 39.2 cm. According to some embodiments, the length of segment 110 of the endovascular device is at least 39.3 cm. According to some embodiments, the length of segment 110 of the endovascular device is at least 39.4 cm. According to some embodiments, the length of segment 110 of the endovascular device is at least 39.5 cm. According to some embodiments, the length of segment 110 of the endovascular device is at least 39.6 cm. According to some embodiments, the length of segment 110 of the endovascular device is at least 39.7 cm. According to some embodiments, the length of segment 110 of the endovascular device is at least 39.8 cm. According to some embodiments, the length of segment 110 of the endovascular device is at least 39.9 cm.

According to some embodiments, the length of segment 110 of the endovascular device is at least 40 cm. According to some embodiments, the length of segment 110 of the endovascular device is at least 40.1 cm. According to some embodiments, the length of segment 110 of the endovascular device is at least 40.2 cm. According to some embodiments, the length of segment 110 of the endovascular device is at least 40.3 cm. According to some embodiments, the length of segment 110 of the endovascular device is at least 40.4 cm. According to some embodiments, the length of segment 110 of the endovascular device is at least 40.5 cm. According to some embodiments, the length of segment 110 of the endovascular device is at least 40.6 cm. According to some embodiments, the length of segment 110 of the endovascular device is at least 40.7 cm. According to some embodiments, the length of segment 110 of the endovascular device is at least 40.8 cm. According to some embodiments, the length of segment 110 of the endovascular device is at least 40.9 cm.

According to some embodiments, the length of segment 110 of the endovascular device is at least 41 cm. According to some embodiments, the length of segment 110 of the endovascular device is at least 41.1 cm. According to some embodiments, the length of segment 110 of the endovascular device is at least 41.2 cm. According to some embodiments, the length of segment 110 of the endovascular device is at least 41.3 cm. According to some embodiments, the length of segment 110 of the endovascular device is at least 41.4 cm. According to some embodiments, the length of segment 110 of the endovascular device is at least 41.5 cm. According to some embodiments, the length of segment 110 of the endovascular device is at least 41.6 cm. According to some embodiments, the length of segment 110 of the endovascular device is at least 41.7 cm. According to some embodiments, the length of segment 110 of the endovascular device is at least 41.8 cm. According to some embodiments, the length of segment 110 of the endovascular device is at least 41.9 cm.

According to some embodiments, the length of segment 110 of the endovascular device is at least 42 cm. According to some embodiments, the length of segment 110 of the endovascular device is at least 42.1 cm. According to some embodiments, the length of segment 110 of the endovascular device is at least 42.2 cm. According to some embodiments, the length of segment 110 of the endovascular device is at least 42.3 cm. According to some embodiments, the length of segment 110 of the endovascular device is at least 42.4 cm. According to some embodiments, the length of segment 110 of the endovascular device is at least 42.5 cm. According to some embodiments, the length of segment 110 of the endovascular device is at least 42.6 cm. According to some embodiments, the length of segment 110 of the endovascular device is at least 42.7 cm. According to some embodiments, the length of segment 110 of the endovascular device is at least 42.8 cm. According to some embodiments, the length of segment 110 of the endovascular device is at least 42.9 cm.

According to some embodiments, the length of segment 110 of the endovascular device is at least 43 cm. According to some embodiments, the length of segment 110 of the endovascular device is at least 43.1 cm. According to some embodiments, the length of segment 110 of the endovascular device is at least 43.2 cm. According to some embodiments, the length of segment 110 of the endovascular device is at least 43.3 cm. According to some embodiments, the length of segment 110 of the endovascular device is at least 43.4 cm. According to some embodiments, the length of segment 110 of the endovascular device is at least 43.5 cm. According to some embodiments, the length of segment 110 of the endovascular device is at least 43.6 cm. According to some embodiments, the length of segment 110 of the endovascular device is at least 43.7 cm. According to some embodiments, the length of segment 110 of the endovascular device is at least 43.8 cm. According to some embodiments, the length of segment 110 of the endovascular device is at least 43.9 cm.

According to some embodiments, the length of segment 110 of the endovascular device is at least 44 cm. According to some embodiments, the length of segment 110 of the endovascular device is at least 44.1 cm. According to some embodiments, the length of segment 110 of the endovascular device is at least 44.2 cm. According to some embodiments, the length of segment 110 of the endovascular device is at least 44.3 cm. According to some embodiments, the length of segment 110 of the endovascular device is at least 44.4 cm. According to some embodiments, the length of segment 110 of the endovascular device is at least 44.5 cm. According to some embodiments, the length of segment 110 of the endovascular device is at least 44.6 cm. According to some embodiments, the length of segment 110 of the endovascular device is at least 44.7 cm. According to some embodiments, the length of segment 110 of the endovascular device is at least 44.8 cm. According to some embodiments, the length of segment 110 of the endovascular device is at least 44.9 cm.

According to some embodiments, the length of segment 110 of the endovascular device is at least 45 cm. According to some embodiments, the length of segment 110 of the endovascular device is at least 45.1 cm. According to some embodiments, the length of segment 110 of the endovascular device is at least 45.2 cm. According to some embodiments, the length of segment 110 of the endovascular device is at least 45.3 cm. According to some embodiments, the length of segment 110 of the endovascular device is at least 45.4 cm. According to some embodiments, the length of segment 110 of the endovascular device is at least 45.5 cm. According to some embodiments, the length of segment 110 of the endovascular device is at least 45.6 cm. According to some embodiments, the length of segment 110 of the endovascular device is at least 45.7 cm. According to some embodiments, the length of segment 110 of the endovascular device is at least 45.8 cm. According to some embodiments, the length of segment 110 of the endovascular device is at least 45.9 cm.

According to some embodiments, the length of segment 110 of the endovascular device is at least 46 cm. According to some embodiments, the length of segment 110 of the endovascular device is at least 46.1 cm. According to some embodiments, the length of segment 110 of the endovascular device is at least 46.2 cm. According to some embodiments, the length of segment 110 of the endovascular device is at least 46.3 cm. According to some embodiments, the length of segment 110 of the endovascular device is at least 46.4 cm. According to some embodiments, the length of segment 110 of the endovascular device is at least 46.5 cm. According to some embodiments, the length of segment 110 of the endovascular device is at least 46.6 cm. According to some embodiments, the length of segment 110 of the endovascular device is at least 46.7 cm. According to some embodiments, the length of segment 110 of the endovascular device is at least 46.8 cm. According to some embodiments, the length of segment 110 of the endovascular device is at least 46.9 cm.

According to some embodiments, the length of segment 110 of the endovascular device is at least 47 cm. According to some embodiments, the length of segment 110 of the endovascular device is at least 47.1 cm. According to some embodiments, the length of segment 110 of the endovascular device is at least 47.2 cm. According to some embodiments, the length of segment 110 of the endovascular device is at least 47.3 cm. According to some embodiments, the length of segment 110 of the endovascular device is at least 47.4 cm. According to some embodiments, the length of segment 110 of the endovascular device is at least 47.5 cm. According to some embodiments, the length of segment 110 of the endovascular device is at least 47.6 cm. According to some embodiments, the length of segment 110 of the endovascular device is at least 47.7 cm. According to some embodiments, the length of segment 110 of the endovascular device is at least 47.8 cm. According to some embodiments, the length of segment 110 of the endovascular device is at least 47.9 cm.

According to some embodiments, the length of segment 110 of the endovascular device is at least 48 cm. According to some embodiments, the length of segment 110 of the endovascular device is at least 48.1 cm. According to some embodiments, the length of segment 110 of the endovascular device is at least 48.2 cm. According to some embodiments, the length of segment 110 of the endovascular device is at least 48.3 cm. According to some embodiments, the length of segment 110 of the endovascular device is at least 48.4 cm. According to some embodiments, the length of segment 110 of the endovascular device is at least 48.5 cm. According to some embodiments, the length of segment 110 of the endovascular device is at least 48.6 cm. According to some embodiments, the length of segment 110 of the endovascular device is at least 48.7 cm. According to some embodiments, the length of segment 110 of the endovascular device is at least 48.8 cm. According to some embodiments, the length of segment 110 of the endovascular device is at least 48.9 cm.

According to some embodiments, the length of segment 110 of the endovascular device is at least 49 cm. According to some embodiments, the length of segment 110 of the endovascular device is at least 49.1 cm. According to some embodiments, the length of segment 110 of the endovascular device is at least 49.2 cm. According to some embodiments, the length of segment 110 of the endovascular device is at least 49.3 cm. According to some embodiments, the length of segment 110 of the endovascular device is at least 49.4 cm. According to some embodiments, the length of segment 110 of the endovascular device is at least 49.5 cm. According to some embodiments, the length of segment 110 of the endovascular device is at least 49.6 cm. According to some embodiments, the length of segment 110 of the endovascular device is at least 49.7 cm. According to some embodiments, the length of segment 110 of the endovascular device is at least 49.8 cm. According to some embodiments, the length of segment 110 of the endovascular device is at least 49.9 cm.

According to some embodiments, the length of segment 110 of the endovascular device is at least 50 cm. According to some embodiments, the length of segment 110 of the endovascular device is at least 50.1 cm. According to some embodiments, the length of segment 110 of the endovascular device is at least 50.2 cm. According to some embodiments, the length of segment 110 of the endovascular device is at least 50.3 cm. According to some embodiments, the length of segment 110 of the endovascular device is at least 50.4 cm. According to some embodiments, the length of segment 110 of the endovascular device is at least 50.5 cm. According to some embodiments, the length of segment 110 of the endovascular device is at least 50.6 cm. According to some embodiments, the length of segment 110 of the endovascular device is at least 50.7 cm. According to some embodiments, the length of segment 110 of the endovascular device is at least 50.8 cm. According to some embodiments, the length of segment 110 of the endovascular device is at least 50.9 cm.

According to some embodiments, the length of segment 110 of the endovascular device is at least 51 cm. According to some embodiments, the length of segment 110 of the endovascular device is at least 51.1 cm. According to some embodiments, the length of segment 110 of the endovascular device is at least 51.2 cm. According to some embodiments, the length of segment 110 of the endovascular device is at least 51.3 cm. According to some embodiments, the length of segment 110 of the endovascular device is at least 51.4 cm. According to some embodiments, the length of segment 110 of the endovascular device is at least 51.5 cm. According to some embodiments, the length of segment 110 of the endovascular device is at least 51.6 cm. According to some embodiments, the length of segment 110 of the endovascular device is at least 51.7 cm. According to some embodiments, the length of segment 110 of the endovascular device is at least 51.8 cm. According to some embodiments, the length of segment 110 of the endovascular device is at least 51.9 cm.

According to some embodiments, the length of segment 110 of the endovascular device is at least 52 cm. According to some embodiments, the length of segment 110 of the endovascular device is at least 52.1 cm. According to some embodiments, the length of segment 110 of the endovascular device is at least 52.2 cm. According to some embodiments, the length of segment 110 of the endovascular device is at least 52.3 cm. According to some embodiments, the length of segment 110 of the endovascular device is at least 52.4 cm. According to some embodiments, the length of segment 110 of the endovascular device is at least 52.5 cm. According to some embodiments, the length of segment 110 of the endovascular device is at least 52.6 cm. According to some embodiments, the length of segment 110 of the endovascular device is at least 52.7 cm. According to some embodiments, the length of segment 110 of the endovascular device is at least 52.8 cm. According to some embodiments, the length of segment 110 of the endovascular device is at least 52.9 cm.

According to some embodiments, the length of segment 110 of the endovascular device is at least 53 cm. According to some embodiments, the length of segment 110 of the endovascular device is at least 53.1 cm. According to some embodiments, the length of segment 110 of the endovascular device is at least 53.2 cm. According to some embodiments, the length of segment 110 of the endovascular device is at least 53.3 cm. According to some embodiments, the length of segment 110 of the endovascular device is at least 53.4 cm. According to some embodiments, the length of segment 110 of the endovascular device is at least 53.5 cm. According to some embodiments, the length of segment 110 of the endovascular device is at least 53.6 cm. According to some embodiments, the length of segment 110 of the endovascular device is at least 53.7 cm. According to some embodiments, the length of segment 110 of the endovascular device is at least 53.8 cm. According to some embodiments, the length of segment 110 of the endovascular device is at least 53.9 cm.

According to some embodiments, the length of segment 110 of the endovascular device is at least 54 cm. According to some embodiments, the length of segment 110 of the endovascular device is at least 54.1 cm. According to some embodiments, the length of segment 110 of the endovascular device is at least 54.2 cm. According to some embodiments, the length of segment 110 of the endovascular device is at least 54.3 cm. According to some embodiments, the length of segment 110 of the endovascular device is at least 54.4 cm. According to some embodiments, the length of segment 110 of the endovascular device is at least 54.5 cm. According to some embodiments, the length of segment 110 of the endovascular device is at least 54.6 cm. According to some embodiments, the length of segment 110 of the endovascular device is at least 54.7 cm. According to some embodiments, the length of segment 110 of the endovascular device is at least 54.8 cm. According to some embodiments, the length of segment 110 of the endovascular device is at least 54.9 cm.

According to some embodiments, the length of segment 110 of the endovascular device is at least 55 cm. According to some embodiments, the length of segment 110 of the endovascular device is at least 55.1 cm. According to some embodiments, the length of segment 110 of the endovascular device is at least 55.2 cm. According to some embodiments, the length of segment 110 of the endovascular device is at least 55.3 cm. According to some embodiments, the length of segment 110 of the endovascular device is at least 55.4 cm. According to some embodiments, the length of segment 110 of the endovascular device is at least 55.5 cm. According to some embodiments, the length of segment 110 of the endovascular device is at least 55.6 cm. According to some embodiments, the length of segment 110 of the endovascular device is at least 55.7 cm. According to some embodiments, the length of segment 110 of the endovascular device is at least 55.8 cm. According to some embodiments, the length of segment 110 of the endovascular device is at least 55.9 cm.

According to some embodiments, the length of segment 110 of the endovascular device is at least 56 cm. According to some embodiments, the length of segment 110 of the endovascular device is at least 56.1 cm. According to some embodiments, the length of segment 110 of the endovascular device is at least 56.2 cm. According to some embodiments, the length of segment 110 of the endovascular device is at least 56.3 cm. According to some embodiments, the length of segment 110 of the endovascular device is at least 56.4 cm. According to some embodiments, the length of segment 110 of the endovascular device is at least 56.5 cm. According to some embodiments, the length of segment 110 of the endovascular device is at least 56.6 cm. According to some embodiments, the length of segment 110 of the endovascular device is at least 56.7 cm. According to some embodiments, the length of segment 110 of the endovascular device is at least 56.8 cm. According to some embodiments, the length of segment 110 of the endovascular device is at least 56.9 cm.

According to some embodiments, the length of segment 110 of the endovascular device is at least 57 cm. According to some embodiments, the length of segment 110 of the endovascular device is at least 57.1 cm. According to some embodiments, the length of segment 110 of the endovascular device is at least 57.2 cm. According to some embodiments, the length of segment 110 of the endovascular device is at least 57.3 cm. According to some embodiments, the length of segment 110 of the endovascular device is at least 57.4 cm. According to some embodiments, the length of segment 110 of the endovascular device is at least 57.5 cm. According to some embodiments, the length of segment 110 of the endovascular device is at least 57.6 cm. According to some embodiments, the length of segment 110 of the endovascular device is at least 57.7 cm. According to some embodiments, the length of segment 110 of the endovascular device is at least 57.8 cm. According to some embodiments, the length of segment 110 of the endovascular device is at least 57.9 cm.

According to some embodiments, the length of segment 110 of the endovascular device is at least 58 cm. According to some embodiments, the length of segment 110 of the endovascular device is at least 58.1 cm. According to some embodiments, the length of segment 110 of the endovascular device is at least 58.2 cm. According to some embodiments, the length of segment 110 of the endovascular device is at least 58.3 cm. According to some embodiments, the length of segment 110 of the endovascular device is at least 58.4 cm. According to some embodiments, the length of segment 110 of the endovascular device is at least 58.5 cm. According to some embodiments, the length of segment 110 of the endovascular device is at least 58.6 cm. According to some embodiments, the length of segment 110 of the endovascular device is at least 58.7 cm. According to some embodiments, the length of segment 110 of the endovascular device is at least 58.8 cm. According to some embodiments, the length of segment 110 of the endovascular device is at least 58.9 cm.

According to some embodiments, the length of segment 110 of the endovascular device is at least 59 cm. According to some embodiments, the length of segment 110 of the endovascular device is at least 59.1 cm. According to some embodiments, the length of segment 110 of the endovascular device is at least 59.2 cm. According to some embodiments, the length of segment 110 of the endovascular device is at least 59.3 cm. According to some embodiments, the length of segment 110 of the endovascular device is at least 59.4 cm. According to some embodiments, the length of segment 110 of the endovascular device is at least 59.5 cm. According to some embodiments, the length of segment 110 of the endovascular device is at least 59.6 cm. According to some embodiments, the length of segment 110 of the endovascular device is at least 59.7 cm. According to some embodiments, the length of segment 110 of the endovascular device is at least 59.8 cm. According to some embodiments, the length of segment 110 of the endovascular device is at least 59.9 cm.

According to some embodiments, the length of segment 110 of the endovascular device is at least 60 cm. According to some embodiments, the length of segment 110 of the endovascular device is at least 60.1 cm. According to some embodiments, the length of segment 110 of the endovascular device is at least 60.2 cm. According to some embodiments, the length of segment 110 of the endovascular device is at least 60.3 cm. According to some embodiments, the length of segment 110 of the endovascular device is at least 60.4 cm. According to some embodiments, the length of segment 110 of the endovascular device is at least 60.5 cm. According to some embodiments, the length of segment 110 of the endovascular device is at least 60.6 cm. According to some embodiments, the length of segment 110 of the endovascular device is at least 60.7 cm. According to some embodiments, the length of segment 110 of the endovascular device is at least 60.8 cm. According to some embodiments, the length of segment 110 of the endovascular device is at least 60.9 cm.

According to some embodiments, the length of segment 110 of the endovascular device is at least 61 cm. According to some embodiments, the length of segment 110 of the endovascular device is at least 61.1 cm. According to some embodiments, the length of segment 110 of the endovascular device is at least 61.2 cm. According to some embodiments, the length of segment 110 of the endovascular device is at least 61.3 cm. According to some embodiments, the length of segment 110 of the endovascular device is at least 61.4 cm. According to some embodiments, the length of segment 110 of the endovascular device is at least 61.5 cm. According to some embodiments, the length of segment 110 of the endovascular device is at least 61.6 cm. According to some embodiments, the length of segment 110 of the endovascular device is at least 61.7 cm. According to some embodiments, the length of segment 110 of the endovascular device is at least 61.8 cm. According to some embodiments, the length of segment 110 of the endovascular device is at least 61.9 cm.

According to some embodiments, the length of segment 110 of the endovascular device is at least 62 cm. According to some embodiments, the length of segment 110 of the endovascular device is at least 62.1 cm. According to some embodiments, the length of segment 110 of the endovascular device is at least 62.2 cm. According to some embodiments, the length of segment 110 of the endovascular device is at least 62.3 cm. According to some embodiments, the length of segment 110 of the endovascular device is at least 62.4 cm. According to some embodiments, the length of segment 110 of the endovascular device is at least 62.5 cm. According to some embodiments, the length of segment 110 of the endovascular device is at least 62.6 cm. According to some embodiments, the length of segment 110 of the endovascular device is at least 62.7 cm. According to some embodiments, the length of segment 110 of the endovascular device is at least 62.8 cm. According to some embodiments, the length of segment 110 of the endovascular device is at least 62.9 cm.

According to some embodiments, the length of segment 110 of the endovascular device is at least 63 cm. According to some embodiments, the length of segment 110 of the endovascular device is at least 63.1 cm. According to some embodiments, the length of segment 110 of the endovascular device is at least 63.2 cm. According to some embodiments, the length of segment 110 of the endovascular device is at least 63.3 cm. According to some embodiments, the length of segment 110 of the endovascular device is at least 63.4 cm. According to some embodiments, the length of segment 110 of the endovascular device is at least 63.5 cm. According to some embodiments, the length of segment 110 of the endovascular device is at least 63.6 cm. According to some embodiments, the length of segment 110 of the endovascular device is at least 63.7 cm. According to some embodiments, the length of segment 110 of the endovascular device is at least 63.8 cm. According to some embodiments, the length of segment 110 of the endovascular device is at least 63.9 cm.

According to some embodiments, the length of segment 110 of the endovascular device is at least 64 cm. According to some embodiments, the length of segment 110 of the endovascular device is at least 64.1 cm. According to some embodiments, the length of segment 110 of the endovascular device is at least 64.2 cm. According to some embodiments, the length of segment 110 of the endovascular device is at least 64.3 cm. According to some embodiments, the length of segment 110 of the endovascular device is at least 64.4 cm. According to some embodiments, the length of segment 110 of the endovascular device is at least 64.5 cm. According to some embodiments, the length of segment 110 of the endovascular device is at least 64.6 cm. According to some embodiments, the length of segment 110 of the endovascular device is at least 64.7 cm. According to some embodiments, the length of segment 110 of the endovascular device is at least 64.8 cm. According to some embodiments, the length of segment 110 of the endovascular device is at least 64.9 cm.

According to some embodiments, the length of segment 110 of the endovascular device is at least 65 cm. According to some embodiments, the length of segment 110 of the endovascular device is at least 65.1 cm. According to some embodiments, the length of segment 110 of the endovascular device is at least 65.2 cm. According to some embodiments, the length of segment 110 of the endovascular device is at least 65.3 cm. According to some embodiments, the length of segment 110 of the endovascular device is at least 65.4 cm. According to some embodiments, the length of segment 110 of the endovascular device is at least 65.5 cm. According to some embodiments, the length of segment 110 of the endovascular device is at least 65.6 cm. According to some embodiments, the length of segment 110 of the endovascular device is at least 65.7 cm. According to some embodiments, the length of segment 110 of the endovascular device is at least 65.8 cm. According to some embodiments, the length of segment 110 of the endovascular device is at least 65.9 cm.

According to some embodiments, the length of segment 110 of the endovascular device is at least 66 cm. According to some embodiments, the length of segment 110 of the endovascular device is at least 66.1 cm. According to some embodiments, the length of segment 110 of the endovascular device is at least 66.2 cm. According to some embodiments, the length of segment 110 of the endovascular device is at least 66.3 cm. According to some embodiments, the length of segment 110 of the endovascular device is at least 66.4 cm. According to some embodiments, the length of segment 110 of the endovascular device is at least 66.5 cm. According to some embodiments, the length of segment 110 of the endovascular device is at least 66.6 cm. According to some embodiments, the length of segment 110 of the endovascular device is at least 66.7 cm. According to some embodiments, the length of segment 110 of the endovascular device is at least 66.8 cm. According to some embodiments, the length of segment 110 of the endovascular device is at least 66.9 cm.

According to some embodiments, the length of segment 110 of the endovascular device is at least 67 cm. According to some embodiments, the length of segment 110 of the endovascular device is at least 67.1 cm. According to some embodiments, the length of segment 110 of the endovascular device is at least 67.2 cm. According to some embodiments, the length of segment 110 of the endovascular device is at least 67.3 cm. According to some embodiments, the length of segment 110 of the endovascular device is at least 67.4 cm. According to some embodiments, the length of segment 110 of the endovascular device is at least 67.5 cm. According to some embodiments, the length of segment 110 of the endovascular device is at least 67.6 cm. According to some embodiments, the length of segment 110 of the endovascular device is at least 67.7 cm. According to some embodiments, the length of segment 110 of the endovascular device is at least 67.8 cm. According to some embodiments, the length of segment 110 of the endovascular device is at least 67.9 cm.

According to some embodiments, the length of segment 110 of the endovascular device is at least 68 cm. According to some embodiments, the length of segment 110 of the endovascular device is at least 68.1 cm. According to some embodiments, the length of segment 110 of the endovascular device is at least 68.2 cm. According to some embodiments, the length of segment 110 of the endovascular device is at least 68.3 cm. According to some embodiments, the length of segment 110 of the endovascular device is at least 68.4 cm. According to some embodiments, the length of segment 110 of the endovascular device is at least 68.5 cm. According to some embodiments, the length of segment 110 of the endovascular device is at least 68.6 cm. According to some embodiments, the length of segment 110 of the endovascular device is at least 68.7 cm. According to some embodiments, the length of segment 110 of the endovascular device is at least 68.8 cm. According to some embodiments, the length of segment 110 of the endovascular device is at least 68.9 cm.

According to some embodiments, the length of segment 110 of the endovascular device is at least 69 cm. According to some embodiments, the length of segment 110 of the endovascular device is at least 69.1 cm. According to some embodiments, the length of segment 110 of the endovascular device is at least 69.2 cm. According to some embodiments, the length of segment 110 of the endovascular device is at least 69.3 cm. According to some embodiments, the length of segment 110 of the endovascular device is at least 69.4 cm. According to some embodiments, the length of segment 110 of the endovascular device is at least 69.5 cm. According to some embodiments, the length of segment 110 of the endovascular device is at least 69.6 cm. According to some embodiments, the length of segment 110 of the endovascular device is at least 69.7 cm. According to some embodiments, the length of segment 110 of the endovascular device is at least 69.8 cm. According to some embodiments, the length of segment 110 of the endovascular device is at least 69.9 cm.

According to some embodiments, the length of segment 110 of the endovascular device is at least 70 cm. According to some embodiments, the length of segment 110 of the endovascular device is at least 70.1 cm. According to some embodiments, the length of segment 110 of the endovascular device is at least 70.2 cm. According to some embodiments, the length of segment 110 of the endovascular device is at least 70.3 cm. According to some embodiments, the length of segment 110 of the endovascular device is at least 70.4 cm. According to some embodiments, the length of segment 110 of the endovascular device is at least 70.5 cm. According to some embodiments, the length of segment 110 of the endovascular device is at least 70.6 cm. According to some embodiments, the length of segment 110 of the endovascular device is at least 70.7 cm. According to some embodiments, the length of segment 110 of the endovascular device is at least 70.8 cm. According to some embodiments, the length of segment 110 of the endovascular device is at least 70.9 cm.

According to some embodiments, the length of segment 110 of the endovascular device is at least 71 cm. According to some embodiments, the length of segment 110 of the endovascular device is at least 71.1 cm. According to some embodiments, the length of segment 110 of the endovascular device is at least 71.2 cm. According to some embodiments, the length of segment 110 of the endovascular device is at least 71.3 cm. According to some embodiments, the length of segment 110 of the endovascular device is at least 71.4 cm. According to some embodiments, the length of segment 110 of the endovascular device is at least 71.5 cm. According to some embodiments, the length of segment 110 of the endovascular device is at least 71.6 cm. According to some embodiments, the length of segment 110 of the endovascular device is at least 71.7 cm. According to some embodiments, the length of segment 110 of the endovascular device is at least 71.8 cm. According to some embodiments, the length of segment 110 of the endovascular device is at least 71.9 cm.

According to some embodiments, the length of segment 110 of the endovascular device is at least 72 cm. According to some embodiments, the length of segment 110 of the endovascular device is at least 72.1 cm. According to some embodiments, the length of segment 110 of the endovascular device is at least 72.2 cm. According to some embodiments, the length of segment 110 of the endovascular device is at least 72.3 cm. According to some embodiments, the length of segment 110 of the endovascular device is at least 72.4 cm. According to some embodiments, the length of segment 110 of the endovascular device is at least 72.5 cm. According to some embodiments, the length of segment 110 of the endovascular device is at least 72.6 cm. According to some embodiments, the length of segment 110 of the endovascular device is at least 72.7 cm. According to some embodiments, the length of segment 110 of the endovascular device is at least 72.8 cm. According to some embodiments, the length of segment 110 of the endovascular device is at least 72.9 cm.

According to some embodiments, the length of segment 110 of the endovascular device is at least 73 cm. According to some embodiments, the length of segment 110 of the endovascular device is at least 73.1 cm. According to some embodiments, the length of segment 110 of the endovascular device is at least 73.2 cm. According to some embodiments, the length of segment 110 of the endovascular device is at least 73.3 cm. According to some embodiments, the length of segment 110 of the endovascular device is at least 73.4 cm. According to some embodiments, the length of segment 110 of the endovascular device is at least 73.5 cm. According to some embodiments, the length of segment 110 of the endovascular device is at least 73.6 cm. According to some embodiments, the length of segment 110 of the endovascular device is at least 73.7 cm. According to some embodiments, the length of segment 110 of the endovascular device is at least 73.8 cm. According to some embodiments, the length of segment 110 of the endovascular device is at least 73.9 cm.

According to some embodiments, the length of segment 110 of the endovascular device is at least 74 cm. According to some embodiments, the length of segment 110 of the endovascular device is at least 74.1 cm. According to some embodiments, the length of segment 110 of the endovascular device is at least 74.2 cm. According to some embodiments, the length of segment 110 of the endovascular device is at least 74.3 cm. According to some embodiments, the length of segment 110 of the endovascular device is at least 74.4 cm. According to some embodiments, the length of segment 110 of the endovascular device is at least 74.5 cm. According to some embodiments, the length of segment 110 of the endovascular device is at least 74.6 cm. According to some embodiments, the length of segment 110 of the endovascular device is at least 74.7 cm. According to some embodiments, the length of segment 110 of the endovascular device is at least 74.8 cm. According to some embodiments, the length of segment 110 of the endovascular device is at least 74.9 cm.

According to some embodiments, the length of segment 110 of the endovascular device is at least 75 cm. According to some embodiments, the length of segment 110 of the endovascular device is at least 75.1 cm. According to some embodiments, the length of segment 110 of the endovascular device is at least 75.2 cm. According to some embodiments, the length of segment 110 of the endovascular device is at least 75.3 cm. According to some embodiments, the length of segment 110 of the endovascular device is at least 75.4 cm. According to some embodiments, the length of segment 110 of the endovascular device is at least 75.5 cm. According to some embodiments, the length of segment 110 of the endovascular device is at least 75.6 cm. According to some embodiments, the length of segment 110 of the endovascular device is at least 75.7 cm. According to some embodiments, the length of segment 110 of the endovascular device is at least 75.8 cm. According to some embodiments, the length of segment 110 of the endovascular device is at least 75.9 cm.

According to some embodiments, the length of segment 110 of the endovascular device is at least 76 cm. According to some embodiments, the length of segment 110 of the endovascular device is at least 76.1 cm. According to some embodiments, the length of segment 110 of the endovascular device is at least 76.2 cm. According to some embodiments, the length of segment 110 of the endovascular device is at least 76.3 cm. According to some embodiments, the length of segment 110 of the endovascular device is at least 76.4 cm. According to some embodiments, the length of segment 110 of the endovascular device is at least 76.5 cm. According to some embodiments, the length of segment 110 of the endovascular device is at least 76.6 cm. According to some embodiments, the length of segment 110 of the endovascular device is at least 76.7 cm. According to some embodiments, the length of segment 110 of the endovascular device is at least 76.8 cm. According to some embodiments, the length of segment 110 of the endovascular device is at least 76.9 cm.

According to some embodiments, the length of segment 110 of the endovascular device is at least 77 cm. According to some embodiments, the length of segment 110 of the endovascular device is at least 77.1 cm. According to some embodiments, the length of segment 110 of the endovascular device is at least 77.2 cm. According to some embodiments, the length of segment 110 of the endovascular device is at least 77.3 cm. According to some embodiments, the length of segment 110 of the endovascular device is at least 77.4 cm. According to some embodiments, the length of segment 110 of the endovascular device is at least 77.5 cm. According to some embodiments, the length of segment 110 of the endovascular device is at least 77.6 cm. According to some embodiments, the length of segment 110 of the endovascular device is at least 77.7 cm. According to some embodiments, the length of segment 110 of the endovascular device is at least 77.8 cm. According to some embodiments, the length of segment 110 of the endovascular device is at least 77.9 cm.

According to some embodiments, the length of segment 110 of the endovascular device is at least 78 cm. According to some embodiments, the length of segment 110 of the endovascular device is at least 78.1 cm. According to some embodiments, the length of segment 110 of the endovascular device is at least 78.2 cm. According to some embodiments, the length of segment 110 of the endovascular device is at least 78.3 cm. According to some embodiments, the length of segment 110 of the endovascular device is at least 78.4 cm. According to some embodiments, the length of segment 110 of the endovascular device is at least 78.5 cm. According to some embodiments, the length of segment 110 of the endovascular device is at least 78.6 cm. According to some embodiments, the length of segment 110 of the endovascular device is at least 78.7 cm. According to some embodiments, the length of segment 110 of the endovascular device is at least 78.8 cm. According to some embodiments, the length of segment 110 of the endovascular device is at least 78.9 cm.

According to some embodiments, the length of segment 110 of the endovascular device is at least 79 cm. According to some embodiments, the length of segment 110 of the endovascular device is at least 79.1 cm. According to some embodiments, the length of segment 110 of the endovascular device is at least 79.2 cm. According to some embodiments, the length of segment 110 of the endovascular device is at least 79.3 cm. According to some embodiments, the length of segment 110 of the endovascular device is at least 79.4 cm. According to some embodiments, the length of segment 110 of the endovascular device is at least 79.5 cm. According to some embodiments, the length of segment 110 of the endovascular device is at least 79.6 cm. According to some embodiments, the length of segment 110 of the endovascular device is at least 79.7 cm. According to some embodiments, the length of segment 110 of the endovascular device is at least 79.8 cm. According to some embodiments, the length of segment 110 of the endovascular device is at least 79.9 cm.

According to some embodiments, the length of segment 110 of the endovascular device is at least 80 cm. According to some embodiments, the length of segment 110 of the endovascular device is at least 80.1 cm. According to some embodiments, the length of segment 110 of the endovascular device is at least 80.2 cm. According to some embodiments, the length of segment 110 of the endovascular device is at least 80.3 cm. According to some embodiments, the length of segment 110 of the endovascular device is at least 80.4 cm. According to some embodiments, the length of segment 110 of the endovascular device is at least 80.5 cm. According to some embodiments, the length of segment 110 of the endovascular device is at least 80.6 cm. According to some embodiments, the length of segment 110 of the endovascular device is at least 80.7 cm. According to some embodiments, the length of segment 110 of the endovascular device is at least 80.8 cm. According to some embodiments, the length of segment 110 of the endovascular device is at least 80.9 cm.

According to some embodiments, the length of segment 110 of the endovascular device is at least 81 cm. According to some embodiments, the length of segment 110 of the endovascular device is at least 81.1 cm. According to some embodiments, the length of segment 110 of the endovascular device is at least 81.2 cm. According to some embodiments, the length of segment 110 of the endovascular device is at least 81.3 cm. According to some embodiments, the length of segment 110 of the endovascular device is at least 81.4 cm. According to some embodiments, the length of segment 110 of the endovascular device is at least 81.5 cm. According to some embodiments, the length of segment 110 of the endovascular device is at least 81.6 cm. According to some embodiments, the length of segment 110 of the endovascular device is at least 81.7 cm. According to some embodiments, the length of segment 110 of the endovascular device is at least 81.8 cm. According to some embodiments, the length of segment 110 of the endovascular device is at least 81.9 cm.

According to some embodiments, the length of segment 110 of the endovascular device is at least 82 cm. According to some embodiments, the length of segment 110 of the endovascular device is at least 82.1 cm. According to some embodiments, the length of segment 110 of the endovascular device is at least 82.2 cm. According to some embodiments, the length of segment 110 of the endovascular device is at least 82.3 cm. According to some embodiments, the length of segment 110 of the endovascular device is at least 82.4 cm. According to some embodiments, the length of segment 110 of the endovascular device is at least 82.5 cm. According to some embodiments, the length of segment 110 of the endovascular device is at least 82.6 cm. According to some embodiments, the length of segment 110 of the endovascular device is at least 82.7 cm. According to some embodiments, the length of segment 110 of the endovascular device is at least 82.8 cm. According to some embodiments, the length of segment 110 of the endovascular device is at least 82.9 cm.

According to some embodiments, the length of segment 110 of the endovascular device is at least 83 cm. According to some embodiments, the length of segment 110 of the endovascular device is at least 83.1 cm. According to some embodiments, the length of segment 110 of the endovascular device is at least 83.2 cm. According to some embodiments, the length of segment 110 of the endovascular device is at least 83.3 cm. According to some embodiments, the length of segment 110 of the endovascular device is at least 83.4 cm. According to some embodiments, the length of segment 110 of the endovascular device is at least 83.5 cm. According to some embodiments, the length of segment 110 of the endovascular device is at least 83.6 cm. According to some embodiments, the length of segment 110 of the endovascular device is at least 83.7 cm. According to some embodiments, the length of segment 110 of the endovascular device is at least 83.8 cm. According to some embodiments, the length of segment 110 of the endovascular device is at least 83.9 cm.

According to some embodiments, the length of segment 110 of the endovascular device is at least 84 cm. According to some embodiments, the length of segment 110 of the endovascular device is at least 84.1 cm. According to some embodiments, the length of segment 110 of the endovascular device is at least 84.2 cm. According to some embodiments, the length of segment 110 of the endovascular device is at least 84.3 cm. According to some embodiments, the length of segment 110 of the endovascular device is at least 84.4 cm. According to some embodiments, the length of segment 110 of the endovascular device is at least 84.5 cm. According to some embodiments, the length of segment 110 of the endovascular device is at least 84.6 cm. According to some embodiments, the length of segment 110 of the endovascular device is at least 84.7 cm. According to some embodiments, the length of segment 110 of the endovascular device is at least 84.8 cm. According to some embodiments, the length of segment 110 of the endovascular device is at least 84.9 cm.

According to some embodiments, the length of segment 110 of the endovascular device is at least 85 cm. According to some embodiments, the length of segment 110 of the endovascular device is at least 85.1 cm. According to some embodiments, the length of segment 110 of the endovascular device is at least 85.2 cm. According to some embodiments, the length of segment 110 of the endovascular device is at least 85.3 cm. According to some embodiments, the length of segment 110 of the endovascular device is at least 85.4 cm. According to some embodiments, the length of segment 110 of the endovascular device is at least 85.5 cm. According to some embodiments, the length of segment 110 of the endovascular device is at least 85.6 cm. According to some embodiments, the length of segment 110 of the endovascular device is at least 85.7 cm. According to some embodiments, the length of segment 110 of the endovascular device is at least 85.8 cm. According to some embodiments, the length of segment 110 of the endovascular device is at least 85.9 cm.

According to some embodiments, the length of segment 110 of the endovascular device is at least 86 cm. According to some embodiments, the length of segment 110 of the endovascular device is at least 86.1 cm. According to some embodiments, the length of segment 110 of the endovascular device is at least 86.2 cm. According to some embodiments, the length of segment 110 of the endovascular device is at least 86.3 cm. According to some embodiments, the length of segment 110 of the endovascular device is at least 86.4 cm. According to some embodiments, the length of segment 110 of the endovascular device is at least 86.5 cm. According to some embodiments, the length of segment 110 of the endovascular device is at least 86.6 cm. According to some embodiments, the length of segment 110 of the endovascular device is at least 86.7 cm. According to some embodiments, the length of segment 110 of the endovascular device is at least 86.8 cm. According to some embodiments, the length of segment 110 of the endovascular device is at least 86.9 cm.

According to some embodiments, the length of segment 110 of the endovascular device is at least 87 cm. According to some embodiments, the length of segment 110 of the endovascular device is at least 87.1 cm. According to some embodiments, the length of segment 110 of the endovascular device is at least 87.2 cm. According to some embodiments, the length of segment 110 of the endovascular device is at least 87.3 cm. According to some embodiments, the length of segment 110 of the endovascular device is at least 87.4 cm. According to some embodiments, the length of segment 110 of the endovascular device is at least 87.5 cm. According to some embodiments, the length of segment 110 of the endovascular device is at least 87.6 cm. According to some embodiments, the length of segment 110 of the endovascular device is at least 87.7 cm. According to some embodiments, the length of segment 110 of the endovascular device is at least 87.8 cm. According to some embodiments, the length of segment 110 of the endovascular device is at least 87.9 cm.

According to some embodiments, the length of segment 110 of the endovascular device is at least 88 cm. According to some embodiments, the length of segment 110 of the endovascular device is at least 88.1 cm. According to some embodiments, the length of segment 110 of the endovascular device is at least 88.2 cm. According to some embodiments, the length of segment 110 of the endovascular device is at least 88.3 cm. According to some embodiments, the length of segment 110 of the endovascular device is at least 88.4 cm. According to some embodiments, the length of segment 110 of the endovascular device is at least 88.5 cm. According to some embodiments, the length of segment 110 of the endovascular device is at least 88.6 cm. According to some embodiments, the length of segment 110 of the endovascular device is at least 88.7 cm. According to some embodiments, the length of segment 110 of the endovascular device is at least 88.8 cm. According to some embodiments, the length of segment 110 of the endovascular device is at least 88.9 cm.

According to some embodiments, the length of segment 110 of the endovascular device is at least 89 cm. According to some embodiments, the length of segment 110 of the endovascular device is at least 89.1 cm. According to some embodiments, the length of segment 110 of the endovascular device is at least 89.2 cm. According to some embodiments, the length of segment 110 of the endovascular device is at least 89.3 cm. According to some embodiments, the length of segment 110 of the endovascular device is at least 89.4 cm. According to some embodiments, the length of segment 110 of the endovascular device is at least 89.5 cm. According to some embodiments, the length of segment 110 of the endovascular device is at least 89.6 cm. According to some embodiments, the length of segment 110 of the endovascular device is at least 89.7 cm. According to some embodiments, the length of segment 110 of the endovascular device is at least 89.8 cm. According to some embodiments, the length of segment 110 of the endovascular device is at least 89.9 cm.

According to some embodiments, the length of segment 110 of the endovascular device is at least 90 cm. According to some embodiments, the length of segment 110 of the endovascular device is at least 90.1 cm. According to some embodiments, the length of segment 110 of the endovascular device is at least 90.2 cm. According to some embodiments, the length of segment 110 of the endovascular device is at least 90.3 cm. According to some embodiments, the length of segment 110 of the endovascular device is at least 90.4 cm. According to some embodiments, the length of segment 110 of the endovascular device is at least 90.5 cm. According to some embodiments, the length of segment 110 of the endovascular device is at least 90.6 cm. According to some embodiments, the length of segment 110 of the endovascular device is at least 90.7 cm. According to some embodiments, the length of segment 110 of the endovascular device is at least 90.8 cm. According to some embodiments, the length of segment 110 of the endovascular device is at least 90.9 cm.

According to some embodiments, the length of segment 110 of the endovascular device is at least 91 cm. According to some embodiments, the length of segment 110 of the endovascular device is at least 91.5 cm. According to some embodiments, the length of segment 110 of the endovascular device is at least 92 cm. According to some embodiments, the length of segment 110 of the endovascular device is at least 92.5 cm. According to some embodiments, the length of segment 110 of the endovascular device is at least 93 cm. According to some embodiments, the length of segment 110 of the endovascular device is at least 93.5 cm. According to some embodiments, the length of segment 110 of the endovascular device is at least 94 cm. According to some embodiments, the length of segment 110 of the endovascular device is at least 94.5 cm. According to some embodiments, the length of segment 110 of the endovascular device is at least 95 cm. According to some embodiments, the length of segment 110 of the endovascular device is at least 95.5 cm. According to some embodiments, the length of segment 110 of the endovascular device is at least 96 cm. According to some embodiments, the length of segment 110 of the endovascular device is at least 96.5 cm. According to some embodiments, the length of segment 110 of the endovascular device is at least 97 cm. According to some embodiments, the length of segment 110 of the endovascular device is at least 97.5 cm. According to some embodiments, the length of segment 110 of the endovascular device is at least 98 cm. According to some embodiments, the length of segment 110 of the endovascular device is at least 98.5 cm. According to some embodiments, the length of segment 110 of the endovascular device is at least 99 cm. According to some embodiments, the length of segment 110 of the endovascular device is at least 99.5 cm. According to some embodiments, the length of segment 110 of the endovascular device is at least 100 cm. According to some embodiments, the length of segment 110 of the endovascular device is at least 100.5 cm. According to some embodiments, the length of segment 110 of the endovascular device is at least 101 cm. According to some embodiments, the length of segment 110 of the endovascular device is at least 101.5 cm. According to some embodiments, the length of segment 110 of the endovascular device is at least 102 cm. According to some embodiments, the length of segment 110 of the endovascular device is at least 102.5 cm. According to some embodiments, the length of segment 110 of the endovascular device is at least 103 cm. According to some embodiments, the length of segment 110 of the endovascular device is at least 103.5 cm. According to some embodiments, the length of segment 110 of the endovascular device is at least 104 cm. According to some embodiments, the length of segment 110 of the endovascular device is at least 104.5 cm. According to some embodiments, the length of segment 110 of the endovascular device is at least 105 cm. According to some embodiments, the length of segment 110 of the endovascular device is at least 105.5 cm. According to some embodiments, the length of segment 110 of the endovascular device is at least 106 cm. According to some embodiments, the length of segment 110 of the endovascular device is at least 106.5 cm. According to some embodiments, the length of segment 110 of the endovascular device is at least 107 cm. According to some embodiments, the length of segment 110 of the endovascular device is at least 107.5 cm. According to some embodiments, the length of segment 110 of the endovascular device is at least 108 cm. According to some embodiments, the length of segment 110 of the endovascular device is at least 108.5 cm. According to some embodiments, the length of segment 110 of the endovascular device is at least 109 cm. According to some embodiments, the length of segment 110 of the endovascular device is at least 109.5 cm. According to some embodiments, the length of segment 110 of the endovascular device is at least 110 cm. According to some embodiments, the length of segment 110 of the endovascular device is at least 110.5 cm. According to some embodiments, the length of segment 110 of the endovascular device is at least 111 cm. According to some embodiments, the length of segment 110 of the endovascular device is at least 111.5 cm. According to some embodiments, the length of segment 110 of the endovascular device is at least 112 cm. According to some embodiments, the length of segment 110 of the endovascular device is at least 112.5 cm. According to some embodiments, the length of segment 110 of the endovascular device is at least 113 cm. According to some embodiments, the length of segment 110 of the endovascular device is at least 113.5 cm. According to some embodiments, the length of segment 110 of the endovascular device is at least 114 cm. According to some embodiments, the length of segment 110 of the endovascular device is at least 114.5 cm. According to some embodiments, the length of segment 110 of the endovascular device is at least 115 cm. According to some embodiments, the length of segment 110 of the endovascular device is at least 115.5 cm. According to some embodiments, the length of segment 110 of the endovascular device is at least 116 cm. According to some embodiments, the length of segment 110 of the endovascular device is at least 116.5 cm. According to some embodiments, the length of segment 110 of the endovascular device is at least 117 cm. According to some embodiments, the length of segment 110 of the endovascular device is at least 117.5 cm. According to some embodiments, the length of segment 110 of the endovascular device is at least 118 cm. According to some embodiments, the length of segment 110 of the endovascular device is at least 118.5 cm. According to some embodiments, the length of segment 110 of the endovascular device is at least 119 cm. According to some embodiments, the length of segment 110 of the endovascular device is at least 119.5 cm. According to some embodiments, the length of segment 110 of the endovascular device is at least 120 cm. According to some embodiments, the length of segment 110 of the endovascular device is at least 120.5 cm. According to some embodiments, the length of segment 110 of the endovascular device is at least 121 cm. According to some embodiments, the length of segment 110 of the endovascular device is at least 121.5 cm. According to some embodiments, the length of segment 110 of the endovascular device is at least 122 cm. According to some embodiments, the length of segment 110 of the endovascular device is at least 122.5 cm. According to some embodiments, the length of segment 110 of the endovascular device is at least 123 cm. According to some embodiments, the length of segment 110 of the endovascular device is at least 123.5 cm. According to some embodiments, the length of segment 110 of the endovascular device is at least 124 cm. According to some embodiments, the length of segment 110 of the endovascular device is at least 124.5 cm. According to some embodiments, the length of segment 110 of the endovascular device is at least 125 cm. According to some embodiments, the length of segment 110 of the endovascular device is at least 125.5 cm. According to some embodiments, the length of segment 110 of the endovascular device is at least 126 cm. According to some embodiments, the length of segment 110 of the endovascular device is at least 126.5 cm. According to some embodiments, the length of segment 110 of the endovascular device is at least 127 cm. According to some embodiments, the length of segment 110 of the endovascular device is at least 127.5 cm. According to some embodiments, the length of segment 110 of the endovascular device is at least 128 cm. According to some embodiments, the length of segment 110 of the endovascular device is at least 128.5 cm. According to some embodiments, the length of segment 110 of the endovascular device is at least 129 cm. According to some embodiments, the length of segment 110 of the endovascular device is at least 129.5 cm. According to some embodiments, the length of segment 110 of the endovascular device is at least 130 cm. According to some embodiments, the length of segment 110 of the endovascular device is at least 130.5 cm. According to some embodiments, the length of segment 110 of the endovascular device is at least 131 cm. According to some embodiments, the length of segment 110 of the endovascular device is at least 131.5 cm. According to some embodiments, the length of segment 110 of the endovascular device is at least 132 cm. According to some embodiments, the length of segment 110 of the endovascular device is at least 132.5 cm. According to some embodiments, the length of segment 110 of the endovascular device is at least 133 cm. According to some embodiments, the length of segment 110 of the endovascular device is at least 133.5 cm. According to some embodiments, the length of segment 110 of the endovascular device is at least 134 cm. According to some embodiments, the length of segment 110 of the endovascular device is at least 134.5 cm. According to some embodiments, the length of segment 110 of the endovascular device is at least 135 cm. According to some embodiments, the length of segment 110 of the endovascular device is at least 135.5 cm. According to some embodiments, the length of segment 110 of the endovascular device is at least 136 cm. According to some embodiments, the length of segment 110 of the endovascular device is at least 136.5 cm. According to some embodiments, the length of segment 110 of the endovascular device is at least 137 cm. According to some embodiments, the length of segment 110 of the endovascular device is at least 137.5 cm. According to some embodiments, the length of segment 110 of the endovascular device is at least 138 cm. According to some embodiments, the length of segment 110 of the endovascular device is at least 138.5 cm. According to some embodiments, the length of segment 110 of the endovascular device is at least 139 cm. According to some embodiments, the length of segment 110 of the endovascular device is at least 139.5 cm. According to some embodiments, the length of segment 110 of the endovascular device is at least 140 cm. According to some embodiments, the length of segment 110 of the endovascular device is at least 140.5 cm. According to some embodiments, the length of segment 110 of the endovascular device is at least 141 cm. According to some embodiments, the length of segment 110 of the endovascular device is at least 141.5 cm. According to some embodiments, the length of segment 110 of the endovascular device is at least 142 cm. According to some embodiments, the length of segment 110 of the endovascular device is at least 142.5 cm. According to some embodiments, the length of segment 110 of the endovascular device is at least 143 cm. According to some embodiments, the length of segment 110 of the endovascular device is at least 143.5 cm. According to some embodiments, the length of segment 110 of the endovascular device is at least 144 cm. According to some embodiments, the length of segment 110 of the endovascular device is at least 144.5 cm. According to some embodiments, the length of segment 110 of the endovascular device is at least 145 cm. According to some embodiments, the length of segment 110 of the endovascular device is at least 145.5 cm. According to some embodiments, the length of segment 110 of the endovascular device is at least 146 cm. According to some embodiments, the length of segment 110 of the endovascular device is at least 146.5 cm. According to some embodiments, the length of segment 110 of the endovascular device is at least 147 cm. According to some embodiments, the length of segment 110 of the endovascular device is at least 147.5 cm. According to some embodiments, the length of segment 110 of the endovascular device is at least 148 cm. According to some embodiments, the length of segment 110 of the endovascular device is at least 148.5 cm. According to some embodiments, the length of segment 110 of the endovascular device is at least 149 cm. According to some embodiments, the length of segment 110 of the endovascular device is at least 149.5 cm. According to some embodiments, the length of segment 110 of the endovascular device is at least 150 cm. According to some embodiments, the length of segment 110 of the endovascular device is at least 150.5 cm. According to some embodiments, the length of segment 110 of the endovascular device is at least 151 cm. According to some embodiments, the length of segment 110 of the endovascular device is at least 151.5 cm. According to some embodiments, the length of segment 110 of the endovascular device is at least 152 cm. According to some embodiments, the length of segment 110 of the endovascular device is at least 152.5 cm. According to some embodiments, the length of segment 110 of the endovascular device is at least 153 cm. According to some embodiments, the length of segment 110 of the endovascular device is at least 153.5 cm. According to some embodiments, the length of segment 110 of the endovascular device is at least 154 cm. According to some embodiments, the length of segment 110 of the endovascular device is at least 154.5 cm. According to some embodiments, the length of segment 110 of the endovascular device is at least 155 cm. According to some embodiments, the length of segment 110 of the endovascular device is at least 155.5 cm. According to some embodiments, the length of segment 110 of the endovascular device is at least 156 cm. According to some embodiments, the length of segment 110 of the endovascular device is at least 156.5 cm. According to some embodiments, the length of segment 110 of the endovascular device is at least 157 cm. According to some embodiments, the length of segment 110 of the endovascular device is at least 157.5 cm. According to some embodiments, the length of segment 110 of the endovascular device is at least 158 cm. According to some embodiments, the length of segment 110 of the endovascular device is at least 158.5 cm. According to some embodiments, the length of segment 110 of the endovascular device is at least 159 cm. According to some embodiments, the length of segment 110 of the endovascular device is at least 159.5 cm. According to some embodiments, the length of segment 110 of the endovascular device is at least 160 cm. According to some embodiments, the length of segment 110 of the endovascular device is at least 160.5 cm. According to some embodiments, the length of segment 110 of the endovascular device is at least 161 cm. According to some embodiments, the length of segment 110 of the endovascular device is at least 161.5 cm. According to some embodiments, the length of segment 110 of the endovascular device is at least 162 cm. According to some embodiments, the length of segment 110 of the endovascular device is at least 162.5 cm. According to some embodiments, the length of segment 110 of the endovascular device is at least 163 cm. According to some embodiments, the length of segment 110 of the endovascular device is at least 163.5 cm. According to some embodiments, the length of segment 110 of the endovascular device is at least 164 cm. According to some embodiments, the length of segment 110 of the endovascular device is at least 164.5 cm. According to some embodiments, the length of segment 110 of the endovascular device is at least 165 cm. According to some embodiments, the length of segment 110 of the endovascular device is at least 165.5 cm. According to some embodiments, the length of segment 110 of the endovascular device is at least 166 cm. According to some embodiments, the length of segment 110 of the endovascular device is at least 166.5 cm. According to some embodiments, the length of segment 110 of the endovascular device is at least 167 cm. According to some embodiments, the length of segment 110 of the endovascular device is at least 167.5 cm. According to some embodiments, the length of segment 110 of the endovascular device is at least 168 cm. According to some embodiments, the length of segment 110 of the endovascular device is at least 168.5 cm. According to some embodiments, the length of segment 110 of the endovascular device is at least 169 cm. According to some embodiments, the length of segment 110 of the endovascular device is at least 169.5 cm. According to some embodiments, the length of segment 110 of the endovascular device is at least 170 cm. According to some embodiments, the length of segment 110 of the endovascular device is at least 170.5 cm. According to some embodiments, the length of segment 110 of the endovascular device is at least 171 cm. According to some embodiments, the length of segment 110 of the endovascular device is at least 171.5 cm. According to some embodiments, the length of segment 110 of the endovascular device is at least 172 cm. According to some embodiments, the length of segment 110 of the endovascular device is at least 172.5 cm. According to some embodiments, the length of segment 110 of the endovascular device is at least 173 cm. According to some embodiments, the length of segment 110 of the endovascular device is at least 173.5 cm. According to some embodiments, the length of segment 110 of the endovascular device is at least 174 cm. According to some embodiments, the length of segment 110 of the endovascular device is at least 174.5 cm. According to some embodiments, the length of segment 110 of the endovascular device is at least 175 cm. According to some embodiments, the length of segment 110 of the endovascular device is at least 175.5 cm. According to some embodiments, the length of segment 110 of the endovascular device is at least 176 cm. According to some embodiments, the length of segment 110 of the endovascular device is at least 176.5 cm. According to some embodiments, the length of segment 110 of the endovascular device is at least 177 cm. According to some embodiments, the length of segment 110 of the endovascular device is at least 177.5 cm. According to some embodiments, the length of segment 110 of the endovascular device is at least 178 cm. According to some embodiments, the length of segment 110 of the endovascular device is at least 178.5 cm. According to some embodiments, the length of segment 110 of the endovascular device is at least 179 cm. According to some embodiments, the length of segment 110 of the endovascular device is at least 179.5 cm. According to some embodiments, the length of segment 110 of the endovascular device is at least 180 cm. According to some embodiments, the length of segment 110 of the endovascular device is at least 180.5 cm. According to some embodiments, the length of segment 110 of the endovascular device is at least 181 cm. According to some embodiments, the length of segment 110 of the endovascular device is at least 181.5 cm. According to some embodiments, the length of segment 110 of the endovascular device is at least 182 cm. According to some embodiments, the length of segment 110 of the endovascular device is at least 182.5 cm. According to some embodiments, the length of segment 110 of the endovascular device is at least 183 cm. According to some embodiments, the length of segment 110 of the endovascular device is at least 183.5 cm. According to some embodiments, the length of segment 110 of the endovascular device is at least 184 cm. According to some embodiments, the length of segment 110 of the endovascular device is at least 184.5 cm. According to some embodiments, the length of segment 110 of the endovascular device is at least 185 cm. According to some embodiments, the length of segment 110 of the endovascular device is at least 185.5 cm. According to some embodiments, the length of segment 110 of the endovascular device is at least 186 cm. According to some embodiments, the length of segment 110 of the endovascular device is at least 186.5 cm. According to some embodiments, the length of segment 110 of the endovascular device is at least 187 cm. According to some embodiments, the length of segment 110 of the endovascular device is at least 187.5 cm. According to some embodiments, the length of segment 110 of the endovascular device is at least 188 cm. According to some embodiments, the length of segment 110 of the endovascular device is at least 188.5 cm. According to some embodiments, the length of segment 110 of the endovascular device is at least 189 cm. According to some embodiments, the length of segment 110 of the endovascular device is at least 189.5 cm. According to some embodiments, the length of segment 110 of the endovascular device is at least 190 cm. According to some embodiments, the length of segment 110 of the endovascular device is at least 190.5 cm. According to some embodiments, the length of segment 110 of the endovascular device is at least 191 cm. According to some embodiments, the length of segment 110 of the endovascular device is at least 191.5 cm. According to some embodiments, the length of segment 110 of the endovascular device is at least 192 cm. According to some embodiments, the length of segment 110 of the endovascular device is at least 192.5 cm. According to some embodiments, the length of segment 110 of the endovascular device is at least 193 cm. According to some embodiments, the length of segment 110 of the endovascular device is at least 193.5 cm. According to some embodiments, the length of segment 110 of the endovascular device is at least 194 cm. According to some embodiments, the length of segment 110 of the endovascular device is at least 194.5 cm. According to some embodiments, the length of segment 110 of the endovascular device is at least 195 cm. According to some embodiments, the length of segment 110 of the endovascular device is at least 195.5 cm. According to some embodiments, the length of segment 110 of the endovascular device is at least 196 cm. According to some embodiments, the length of segment 110 of the endovascular device is at least 196.5 cm. According to some embodiments, the length of segment 110 of the endovascular device is at least 197 cm. According to some embodiments, the length of segment 110 of the endovascular device is at least 197.5 cm. According to some embodiments, the length of segment 110 of the endovascular device is at least 198 cm. According to some embodiments, the length of segment 110 of the endovascular device is at least 198.5 cm. According to some embodiments, the length of segment 110 of the endovascular device is at least 199 cm. According to some embodiments, the length of segment 110 of the endovascular device is at least 199.5 cm. According to some embodiments, the length of segment 110 of the endovascular device is at least 200 cm.

According to some embodiments, the diameter of the lumen of distal support segment 195 is less than the diameter of the working lumen 161. Said another way, the diameter of the lumen 161 of working segment 120 is greater than the diameter of the support lumen 171. According to some embodiments, the diameter of the support lumen 171 is equal to the diameter of the working lumen 161. Said another way, the diameter of the support lumen 171 is the same as the diameter of the working lumen 161. According to some embodiments, the diameter is an inner diameter (ID). According to some embodiments, the diameter is an outer diameter (OD).

According to some embodiments, the diameter of the support lumen 172 ranges from about 0.1 French (Fr) to about 30 French (Fr).

According to some embodiments, the diameter of the support lumen 172 is less than 1 French (Fr). According to some embodiments the diameter of the support lumen 172 ranges from at least 0.0020 cm (at least 0.0008 in.) to at least 0.0305 cm (at least 0.038 in.). According to some embodiments the diameter of the support lumen 172 is at least 0.0020 cm (at least 0.0008 inches). According to some embodiments, the diameter of the support lumen 172 is at least 0.0023 cm (at least 0.0009 inches). According to some embodiments, the diameter of the support lumen 172 is at least 0.0254 cm (at least 0.010 inches). According to some embodiments, the diameter of the support lumen 172 is at least 0.0279 cm (at least 0.011 inches). According to some embodiments, the diameter of the support lumen 172 is at least 0.0305 cm (at least 0.038 in.). According to some embodiments, in the single lumen version, the diameter of the support lumen can be as much as 30 Fr., as described above.

According to some embodiments, the diameter of the working lumen 161 is less than 1 French (Fr). According to some embodiments the diameter of the working lumen 161 ranges from at least 0.0152 cm (at least 0.006 inches) to at least 0.0305 cm (at least 30 Fr. inner diameter). According to some embodiments, the diameter of the working lumen 161 is at least 0.0152 cm (at least 0.006 inches). According to some embodiments, the diameter of the working lumen 161 is at least 0.0279 cm (at least 0.011 inches). According to some embodiments, the diameter of the working lumen 161 is at least 0.0305 cm (at least 30 Fr.).

According to some embodiments, the diameter of the working lumen 161 for intracranial applications ranges from about 0.001 in to about 0.11 in.. According to some embodiments, the diameter of the working lumen 161 is at least 0.001 in. According to some embodiments, the diameter of the working lumen 161 is at least 0.002 in. According to some embodiments, the diameter of the working lumen 161 is at least 0.003 in. According to some embodiments, the diameter of the working lumen 161 is at least 0.004 in. According to some embodiments, the diameter of the working lumen 161 is at least 0.005 in. According to some embodiments, the diameter of the working lumen 161 is at least 0.006 in. According to some embodiments, the diameter of the working lumen 161 is at least 0.007 in. According to some embodiments, the diameter of the working lumen 161 is at least 0.008 in. According to some embodiments, the diameter of the working lumen 161 is at least 0.009 in. According to some embodiments, the diameter of the working lumen 161 is at least 0.010 in. According to some embodiments, the diameter of the working lumen 161 is at least 0.011 in. According to some embodiments, the diameter of the working lumen 161 is at least 0.012 in. According to some embodiments, the diameter of the working lumen 161 is at least 0.013 in. According to some embodiments, the diameter of the working lumen 161 is at least 0.014 in. According to some embodiments, the diameter of the working lumen 161 is at least 0.015 in. According to some embodiments, the diameter of the working lumen 161 is at least 0.016 in. According to some embodiments, the diameter of the working lumen 161 is at least 0.017 in. According to some embodiments, the diameter of the working lumen 161 is at least 0.018 in. According to some embodiments, the diameter of the working lumen 161 is at least 0.019 in. According to some embodiments, the diameter of the working lumen 161 is at least 0.020 in. According to some embodiments, the diameter of the working lumen 161 is at least 0.021 in. According to some embodiments, the diameter of the working lumen 161 is at least 0.022 in. According to some embodiments, the diameter of the working lumen 161 is at least 0.023 in. According to some embodiments, the diameter of the working lumen 161 is at least 0.024 in. According to some embodiments, the diameter of the working lumen 161 is at least 0.025 in. According to some embodiments, the diameter of the working lumen 161 is at least 0.026 in. According to some embodiments, the diameter of the working lumen 161 is at least 0.027 in. According to some embodiments, the diameter of the working lumen 161 is at least 0.028 in. According to some embodiments, the diameter of the working lumen 161 is at least 0.029 in. According to some embodiments, the diameter of the working lumen 161 is at least 0.030 in. According to some embodiments, the diameter of the working lumen 161 is at least 0.031 in. According to some embodiments, the diameter of the working lumen 161 is at least 0.032 in. According to some embodiments, the diameter of the working lumen 161 is at least 0.033 in. According to some embodiments, the diameter of the working lumen 161 is at least 0.034 in. According to some embodiments, the diameter of the working lumen 161 is at least 0.035 in. According to some embodiments, the diameter of the working lumen 161 is at least 0.036 in. According to some embodiments, the diameter of the working lumen 161 is at least 0.037 in. According to some embodiments, the diameter of the working lumen 161 is at least 0.038 in. According to some embodiments, the diameter of the working lumen 161 is at least 0.039 in. According to some embodiments, the diameter of the working lumen 161 is at least 0.040 in. According to some embodiments, the diameter of the working lumen 161 is at least 0.041 in. According to some embodiments, the diameter of the working lumen 161 is at least 0.042 in. According to some embodiments, the diameter of the working lumen 161 is at least 0.043 in. According to some embodiments, the diameter of the working lumen 161 is at least 0.044 in. According to some embodiments, the diameter of the working lumen 161 is at least 0.045 in. According to some embodiments, the diameter of the working lumen 161 is at least 0.046 in. According to some embodiments, the diameter of the working lumen 161 is at least 0.047 in. According to some embodiments, the diameter of the working lumen 161 is at least 0.048 in. According to some embodiments, the diameter of the working lumen 161 is at least 0.049 in. According to some embodiments, the diameter of the working lumen 161 is at least 0.050 in. According to some embodiments, the diameter of the working lumen 161 is at least 0.051 in. According to some embodiments, the diameter of the working lumen 161 is at least 0.052 in. According to some embodiments, the diameter of the working lumen 161 is at least 0.053 in. According to some embodiments, the diameter of the working lumen 161 is at least 0.054 in. According to some embodiments, the diameter of the working lumen 161 is at least 0.055 in. According to some embodiments, the diameter of the working lumen 161 is at least 0.056 in. According to some embodiments, the diameter of the working lumen 161 is at least 0.057 in. According to some embodiments, the diameter of the working lumen 161 is at least 0.058 in. According to some embodiments, the diameter of the working lumen 161 is at least 0.059 in. According to some embodiments, the diameter of the working lumen 161 is at least 0.060 in. According to some embodiments, the diameter of the working lumen 161 is at least 0.061 in. According to some embodiments, the diameter of the working lumen 161 is at least 0.062 in. According to some embodiments, the diameter of the working lumen 161 is at least 0.063 in. According to some embodiments, the diameter of the working lumen 161 is at least 0.064 in. According to some embodiments, the diameter of the working lumen 161 is at least 0.065 in. According to some embodiments, the diameter of the working lumen 161 is at least 0.066 in. According to some embodiments, the diameter of the working lumen 161 is at least 0.067 in. According to some embodiments, the diameter of the working lumen 161 is at least 0.068 in. According to some embodiments, the diameter of the working lumen 161 is at least 0.069 in. According to some embodiments, the diameter of the working lumen 161 is at least 0.070 in. According to some embodiments, the diameter of the working lumen 161 is at least 0.071 in. According to some embodiments, the diameter of the working lumen 161 is at least 0.072 in. According to some embodiments, the diameter of the working lumen 161 is at least 0.073 in. According to some embodiments, the diameter of the working lumen 161 is at least 0.074 in. According to some embodiments, the diameter of the working lumen 161 is at least 0.075 in. According to some embodiments, the diameter of the working lumen 161 is at least 0.076 in. According to some embodiments, the diameter of the working lumen 161 is at least 0.077 in. According to some embodiments, the diameter of the working lumen 161 is at least 0.078 in. According to some embodiments, the diameter of the working lumen 161 is at least 0.079 in. According to some embodiments, the diameter of the working lumen 161 is at least 0.080 in. According to some embodiments, the diameter of the working lumen 161 is at least 0.081 in. According to some embodiments, the diameter of the working lumen 161 is at least 0.082 in. According to some embodiments, the diameter of the working lumen 161 is at least 0.083 in. According to some embodiments, the diameter of the working lumen 161 is at least 0.084 in. According to some embodiments, the diameter of the working lumen 161 is at least 0.085 in. According to some embodiments, the diameter of the working lumen 161 is at least 0.086 in. According to some embodiments, the diameter of the working lumen 161 is at least 0.087 in. According to some embodiments, the diameter of the working lumen 161 is at least 0.088 in. According to some embodiments, the diameter of the working lumen 161 is at least 0.089 in. According to some embodiments, the diameter of the working lumen 161 is at least 0.090 in. According to some embodiments, the diameter of the working lumen 161 is at least 0.091 in. According to some embodiments, the diameter of the working lumen 161 is at least 0.092 in. According to some embodiments, the diameter of the working lumen 161 is at least 0.093 in. According to some embodiments, the diameter of the working lumen 161 is at least 0.094 in. According to some embodiments, the diameter of the working lumen 161 is at least 0.095 in. According to some embodiments, the diameter of the working lumen 161 is at least 0.096 in. According to some embodiments, the diameter of the working lumen 161 is at least 0.097 in. According to some embodiments, the diameter of the working lumen 161 is at least 0.098 in. According to some embodiments, the diameter of the working lumen 161 is at least 0.099 in. According to some embodiments, the diameter of the working lumen 161 is at least 0.10 in. According to some embodiments, the diameter of the working lumen 161 is about 0.11 in.

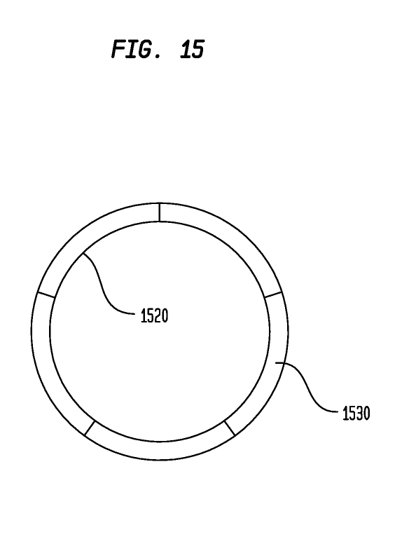

According to some embodiments, the endovascular device 100 comprises one or more fluid delivery tubes defining fluid delivery lumina in addition to the main working lumen 161. According to some embodiments, the one or more fluid delivery lumina provide fluid media to inflate one or more inflatable balloons via a single additional fluid delivery lumen. According to some embodiments, the one or more fluid delivery lumina provide fluid media to inflate one or more inflatable balloons via multiple additional fluid delivery lumina. According to some embodiments, the one or more fluid delivery lumina are effective to remove fluid media to deflate one or more inflatable balloons. According to some embodiments, the fluid delivery tubes defining fluid delivery lumina may be arranged in a side-by side arrangement similar to FIG. 2 (using 171 in this instance as a fluid delivery lumen), concentric to the working lumen (e.g. FIG. 15) in a concentric fashion, or in a combination thereof.

According to some embodiments, endovascular device 100 can comprise additional support elements, for example, a stent, a wire, or a balloon. According to some embodiments, support tube 172 of catheter 100 can be further supported by placing a wire into and through support lumen 171.

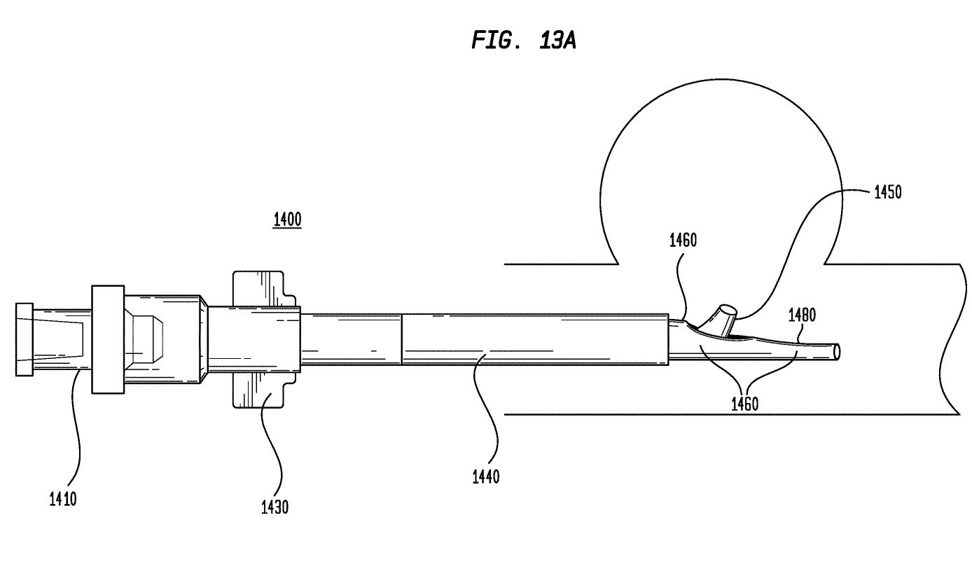

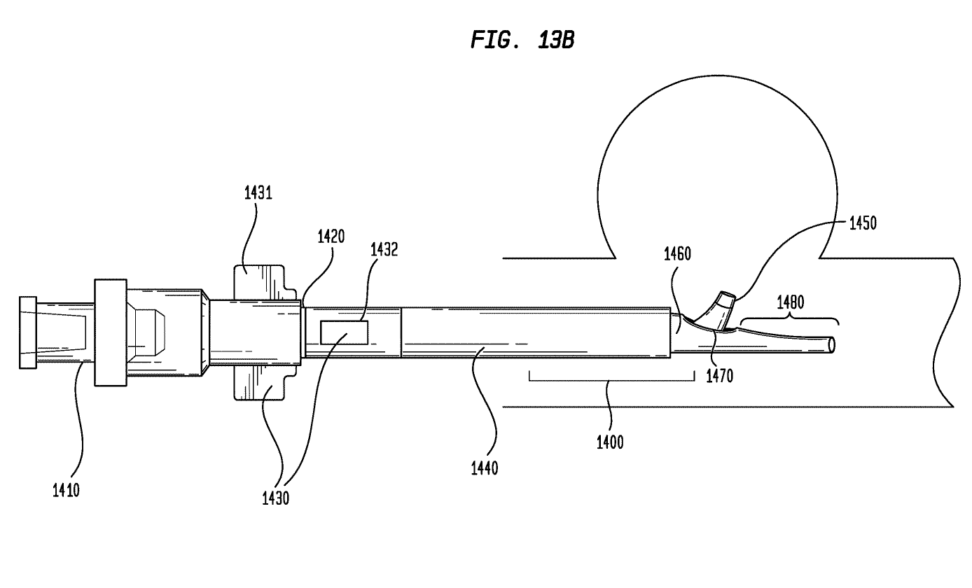

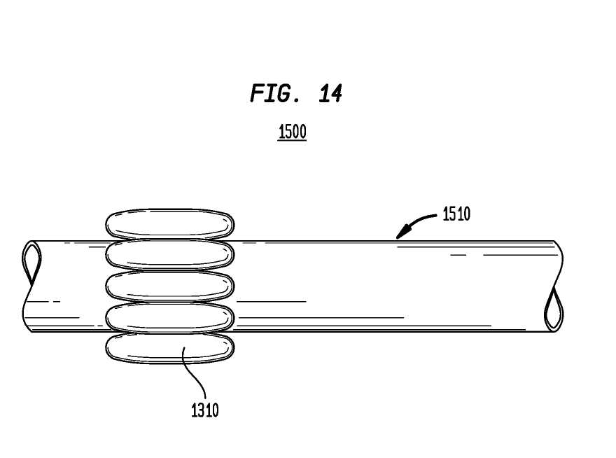

According to some embodiments, working segment 120 and /or support segment 195 (as well as corresponding segments 320 and 340 of FIGS. 3, 1440 and 1460 of FIG. 13A, and 1500 of FIG. 14) of the endovascular device comprises one or more inflatable balloons. According to some embodiments, the one or more inflatable balloons are located on the outside of working segment 120 of the endovascular device. According to some embodiments, at least one (1) inflatable balloon is attached around the circumference of tube 130. According to some embodiments, at least two (2) inflatable balloons are attached around the circumference of tube 130. According to some embodiments, at least three (3) inflatable balloons are attached around the circumference of tube 130. According to some embodiments, at least four (4) inflatable balloons are attached around the circumference of tube 130. According to some embodiments, at least five (5) inflatable balloons are attached around the circumference of tube 130. According to some embodiments, more than five (5) inflatable balloons are attached around the circumference of tube 130. According to some embodiments, the balloons are attached equidistantly from each other around the circumference of tube 130.

According to some embodiments, the inflatable balloons are attached at a distance of about 0.1 cm to about 4 cm from primary opening 160 of tube 130 (and corresponding parts 1210 of FIG. 3, and 1470 of FIG. 13A) . According to some embodiments, the inflatable balloons are attached at a distance of about 0.1 cm from primary opening 160 of tube 130. According to some embodiments, the inflatable balloons are attached at a distance of about 0.2 cm from primary opening 160 of tube 130. According to some embodiments, the inflatable balloons are attached at a distance of about 0.3 cm from primary opening 160 of tube 130. According to some embodiments, the inflatable balloons are attached at a distance of about 0.4 cm from primary opening 160 of tube 130. According to some embodiments, the inflatable balloons are attached at a distance of about 0.5 cm from the primary opening 160 of tube 130. According to some embodiments, the inflatable balloons are attached at a distance of about 0.6 cm from the primary opening 160 of tube 130. According to some embodiments, the inflatable balloons are attached at a distance of about 0.7 cm from primary opening 160 of tube 130. According to some embodiments, the inflatable balloons are attached at a distance of about 0.8 cm from primary opening 160 of tube 130. According to some embodiments, the inflatable balloons are attached at a distance of about 0.9 cm from primary opening 160 of tube 130. According to some embodiments, the inflatable balloons are attached at a distance of about 1.0 cm from primary opening 160 of tube 130. According to some embodiments, the inflatable balloons are attached at a distance of about 1.1 cm from primary opening 160 of tube 130. According to some embodiments, the inflatable balloons are attached at a distance of about 1.2 cm from primary opening 160 of tube 130. According to some embodiments, the inflatable balloons are attached at a distance of about 1.3 cm from primary opening 160 of tube 130. According to some embodiments, the inflatable balloons are attached at a distance of about 1.4 cm from primary opening 160 of tube 130. According to some embodiments, the inflatable balloons are attached at a distance of about 1.5 cm from primary opening 160 of tube 130. According to some embodiments, the inflatable balloons are attached at a distance of about 1.6 cm from primary opening 160 of tube 130. According to some embodiments, the inflatable balloons are attached at a distance of about 1.7 cm from primary opening 160 of tube 130. According to some embodiments, the inflatable balloons are attached at a distance of about 1.8 cm from primary opening 160 of tube 130. According to some embodiments, the inflatable balloons are attached at a distance of about 1.9 cm from primary opening 160 of tube 130. According to some embodiments, the inflatable balloons are attached at a distance of about 2.0 cm from primary opening 160 of tube 130. According to some embodiments, the inflatable balloons are attached at a distance of about 2.1 cm from primary opening 160 of tube 130. According to some embodiments, the inflatable balloons are attached at a distance of about 2.0 cm from primary opening 160 of tube 130. According to some embodiments, the inflatable balloons are attached at a distance of about 2.2 cm from primary opening 160 of tube 130. According to some embodiments, the inflatable balloons are attached at a distance of about 2.3 cm from primary opening 160 of tube 130. According to some embodiments, the inflatable balloons are attached at a distance of about 2.4 cm from primary opening 160 of tube 130. According to some embodiments, the inflatable balloons are attached at a distance of about 2.5 cm from primary opening 160 of tube 130. According to some embodiments, the inflatable balloons are attached at a distance of about 2.6 cm from primary opening 160 of tube 130. According to some embodiments, the inflatable balloons are attached at a distance of about 2.7 cm from primary opening 160 of tube 130. According to some embodiments, the inflatable balloons are attached at a distance of about 2.8 cm from primary opening 160 of tube 130. According to some embodiments, the inflatable balloons are attached at a distance of about 2.9 cm from primary opening 160 of tube 130. According to some embodiments, the inflatable balloons are attached at a distance of about 3.0 cm from primary opening 160 of tube 130. According to some embodiments, the inflatable balloons are attached at a distance of about 3.1 cm from primary opening 160 of tube 130. According to some embodiments, the inflatable balloons are attached at a distance of about 3.2 cm from primary opening 160 of tube 130. According to some embodiments, the inflatable balloons are attached at a distance of about 3.3 cm from primary opening 160 of tube 130. According to some embodiments, the inflatable balloons are attached at a distance of about 3.4 cm from primary opening 160 of tube 130. According to some embodiments, the inflatable balloons are attached at a distance of about 3.5 cm from primary opening 160 of tube 130. According to some embodiments, the inflatable balloons are attached at a distance of about 3.6 cm from primary opening 160 of tube 130. According to some embodiments, the inflatable balloons are attached at a distance of about 3.7.0 cm from primary opening 160 of tube 130. According to some embodiments, the inflatable balloons are attached at a distance of about 3.8.0 cm from primary opening 160 of tube 130. According to some embodiments, the inflatable balloons are attached at a distance of about 3.9 cm from primary opening 160 of tube 130. According to some embodiments, the inflatable balloons are attached at a distance of about 4.0 cm from primary opening 160 of tube 130.

According to some embodiments, the inflatable balloons can be inflated individually. According to some embodiments, more than one of the inflatable balloons can be inflated simultaneously via a single fluid syringe. According to some embodiments, the inflated balloon(s) is/are effective to position tube 130 within a blood vessel. According to some embodiments, the inflated balloon(s) is/are effective to position tube 130 within a blood vessel consistent with an operator's desired position. For example, according to some embodiments, the inflated balloon(s) is/are effective to center tube 130 on the base of an aneurysm. According to some embodiments, the inflated balloon(s) is/are effective to center the access catheter on the base of a side-wall aneurysm. According to some embodiments, the inflated balloon(s) is/are effective to center tube 130 on the base of a side-wall aneurysm close to the neck of the side-wall aneurysm. According to some embodiments, the inflated balloon(s) is/are effective to center tube 130 on the base of a side-wall aneurysm away from the neck of a side-wall aneurysm. According to some embodiments, the inflated balloon(s) is/are effective to center tube 130 catheter on the base of a bifurcation aneurysm. According to some embodiments, the blood vessel is an artery. According to some embodiments, the blood vessel is a vein.

According to some embodiments, the balloons are located proximal to the primary opening 160 (and corresponding parts 1210 of FIGS. 3, and 1470 of FIG. 13A). According to some embodiments, the balloons are located adjacent to the primary opening 160 (and corresponding parts 1210 of FIGS. 3, and 1470 of FIG. 13A), without obstructing the primary opening 160 (and corresponding parts 1210 of FIGS. 3, and 1470 of FIG. 13A). According to some embodiments, the balloons are located distal to the primary opening 160 (and corresponding parts 1210 of FIGS. 3, and 1470 of FIG. 13A).

According to some embodiments, the distal end of distal support segment 195 (as well as e.g., the distal end of corresponding segment 340 of FIG. 3 and distal support segment of 1460 beyond working side-hole 1470 of FIG. 13A) can comprise a single balloon in addition to the circumferential array of positioning balloons. The single distal balloon, which is effective to grip the walls of the vessel so that the catheter is less likely to move, is effective to help anchor the catheter in the vessel.

According to some embodiments, the endovascular device comprises a circumferential array of positioning balloons, a single distal balloon or both. It should be understood that a circumferential array of positioning balloons can be placed on any segment of any catheter, and is not limited to any of the disclosed embodiments.

According to some embodiments, one or more inflatable balloons are attached to a second device separate from the endovascular device 100. According to some embodiments, the second device is a microcatheter, an intermediate catheter, a guide catheter, or any other intravascular device. Any of these intravascular devices can comprise a circumferential array of positioning balloons, a single distal balloon or both as described above.

According to some embodiments, the distal portion of segment 110 of the endovascular device, or distal support segment 195 (or corresponding support segment 390 of FIG. 3 and the distal support segment of FIG. 13A) of the endovascular device comprises a stent. According to some embodiments, the stent provides additional support to endovascular device 100.

According to some embodiments, the stent is retrievable. According to some embodiments, the retrievable stent is attached to distal support segment 195 or the distal portion of segment 110 or corresponding support segments illustrated in FIGS. 3 and 13A) of the endovascular device. According to some embodiments, the retrievable stent is attached to a second device separate from the endovascular device 100. According to some embodiments, the second separate device is capable of being advanced into a blood vessel through one or more of the lumina present in segment 110 of the endovascular device. According to some embodiments, the retrievable stent is effective to anchor working segment 120 and/or distal support segment 195 of the endovascular device to a blood vessel. According to some embodiments, the blood vessel is an artery. According to some embodiments, the blood vessel is a vein.

According to some embodiments, part of or all of segment 110 (as well as corresponding illustrated in FIGS. 3 and 13A), is preformed in form of a geometric shape.

According to some embodiments, first branch 140 comprises a luer lock on its proximal end. According to some embodiments, second branch 150 comprises a luer lock on its proximal end. According to some embodiments, a luer lock is attached on the proximal end of each lumen of each endovascular device.

Exemplary Catheter 1400 of FIG. 13

According to some embodiments, endovascular device 1400 comprises an outer support catheter 1440 and an inner catheter 1460 disposed at least partially within the lumen defined by and outer support catheter 1440 (See, e.g. FIG. 13). According to some embodiments, the inner catheter 1460 comprises a side-hole. According to some embodiments, the side-hole is a working distal side-hole. According to some embodiments, the working distal side hole comprises an angled extension 1450. According to some embodiments, a distal support segment 1480 (corresponding to distal support segment 330 of FIG. 3) that extends beyond the distal working side-hole 1470 (corresponding to working side-hole 310 of FIG. 3, serves to provide additional support and stability to any devices being advanced through it into a lesion or an acutely angled and/or tortuous vessel, and minimizes the tendency of such device and/or its delivery wire to “kick-back” into the parent vessel.

According to some embodiments, one or more radiopaque markers can be used to identify the position of the working distal side hole. According to some embodiments, intravascular ultrasound (IVUS) can be incorporated into the catheter to help identify the position of the working side-hole relative to a lesion such as an aneurysm, a vessel or other targeted anatomy.

According to some embodiments, grips 140 are connected to the inner and outer catheters. The term “grips” as used herein refers to a part or attachment of the endovascular device by which the device is held in the hand. According to some such embodiments, inner catheter 1460 is connected to grips on the inner catheter 1431 at the proximal end of the endovascular device (FIG. 13A). A separate set of grips 1432 is on the proximal end of the outer catheter 1440. According to some embodiments, the grips are used by the operator to rotate the inner catheter 1460 relative to the outer catheter 1440. The operator can use the grips 1432 on proximal end of the outer catheter 1440 to hold the outer catheter in a stable position, while using the grips 1431 on the inner catheter 1460 relative to the fixed outer catheter and thereby reposition the working side hold 1470 in a different desired orientation. According to some embodiments, endovascular device 1400 can be rotated within a blood vessel. According to some embodiments, the rotation of endovascular device 1400 is effective to position inner catheter 1460 within a blood vessel. According to some embodiments, the rotation is effective to position the working side-hole of inner catheter 1460 within a blood vessel. According to some embodiments, the rotation is effective to center the working distal side-hole of inner catheter 1460 on the base of an aneurysm. According to some embodiments, the rotation is effective to center the working distal side-hole of inner catheter 1460 in proper alignment with a blood vessel origin and for support in accessing the blood vessel.

According to some embodiments, endovascular device 1400 comprises one or two sets of grips 1430 at the first (proximal) end of the device (FIG. 13B). According to some embodiments, the grips are used by the operator to rotate the inner or outer catheter of the endovascular device 1400. According to some embodiments, a first set of grips can be connected to inner catheter 1460 and a second set of grips can be connected to outer catheter 1440.

According to some embodiments, the grips are used by an operator to rotate the inner catheter 1460 within outer catheter 1440 of the endovascular device 1400. According to some embodiments, the endovascular device 1400 can be rotated within a blood vessel. According to some embodiments, the rotation is effective to position the inner catheter within a blood vessel. According to some embodiments, the rotation is effective to orient the distal working side-hole of device 1400 in a desired direction and location. According to some embodiments, the rotation is effective to center the working side-hole of the inner catheter on the base of an aneurysm. According to some embodiments, the rotation is effective to center the working side-hole of the inner catheter on the origin of a blood vessel.

By way of example, the grips 1430 of outer support catheter 1440 can be immobilized/held stationary while the grips of inner catheter 1460 are rotated relative to the grips of the outer support catheter. Put another way, the outer support catheter is maintained in a fixed position while rotating the inner catheter relative to the outer support catheter.

According to some embodiments, inner catheter 1460 of endovascular device 1400 is endovascular device 100 or endovascular device 300. While endovascular 100 would allow for more support, it would be bulkier and may be more difficult to rotate than endovascular device 300.

It should be understood that the inner/outer catheter arrangement with grips as described above and shown in FIGS. 13 A and 13B is only one illustrative way to achieve rotation. According to some embodiments, for example, a single balloon on the inner catheter just beside the working side-hole is effective to deflect the catheter away from an aneurysm and provide more working room; a circumferential array of balloons can achieve the same purpose. According to another embodiment, a symmetric or asymmetric balloon arranged so that it can deflect the catheter away from a base/neck of the aneurysm while also occluding a vessel, thereby allowing flow arrest and/or flow reversal and decreasing the tendency for normal flow to deflect the advancing endovascular device.

According to some embodiments, a circumferential array of balloons is attached to the outside of the outer catheter 1440. According to some embodiments, the balloons are attached to the outside of the distal, exposed segment of the inner catheter 1460. According to some embodiments, the circumferential balloons can be on the outer sheeth of the catheter. According to some embodiments, the balloons are attached to both the outside of the outer catheter 1440 and the outside of the distal, exposed segment of the inner catheter 1460. According to some embodiments, inner catheter 1460 comprises one or more grooves. According to some embodiments, the one or more grooves are located around the outer circumference of inner catheter 1460. According to some embodiments, outer catheter 1440 comprises one or more grooves. According to some embodiments, the one or more grooves are located around the inner circumference of outer catheter 1440. According to some embodiments, the grooves allow the inner and outer catheters to rotate relative to each other while preventing movement along the length of one catheter relative to the other catheter. By way of example, the inner catheter can comprise an outer groove along the outside of the inner catheter, centered between two (2) inner grooves on the inner surface of the outer catheter at a corresponding location. According to some nonlimiting embodiments, there can be an inner groove along the inside of the outer catheter, centered between two (2) outer grooves on the outside of the inner catheter. According to some embodiments, the inner and outer catheters may comprise additional grooves for added translational stability.

FIG. 15 shows an illustrative embodiment of a cross-section through the fluid channels supplying an array of five (5) balloons (1310) depicted in an exemplary configuration in side view in FIG. 14 at the proximal end of the endovascular device. This embodiment shows a working lumen (1510) and five (5) fluid channels to the five separate balloons. By inflating some balloons and not others, the catheter can be centered at the desired location within a vessel. According to some such embodiments, a catheter can be so centered at the base of a bifurcation aneurysm, for subsequent delivery of a Web, coils or another device. According to some such embodiments, the balloons can be on an intermediate support catheter, or directly on the delivering microcatheter.

The Exemplary Catheter of FIG. 3

FIG. 3 shows an exemplary and non-limiting example of some embodiments of the endovascular device 300 of the described invention in side view. Endovascular device 300 comprising a tube 360 defining a lumen 370, a side-hole 310, a primary opening 340, and an open end 350. The side-hole 310 divides the endovascular device 300 into two (2) segments: a first segment 320 and a second segment 330. The first segment 320 comprises primary opening 340. First segment 320 extends from primary opening 340 to side-hole 310. First segment 320 forms a working segment 380 comprising primary opening 340, lumen 370, and side hole 310. The second segment 330 extends from the side-hole 310 and can taper (i.e., decrease in diameter) to an end 350. The second segment 330 forms support segment 390 comprising end 350 and lumen 370.

According to some embodiments, at least part of first segment 320 and/or second segment 330 of the endovascular device depicted in FIG. 3 is preformed in form of a geometric shape as described above.

According to some embodiments, the length of the working segment 380 formed between the side-hole 310 and the first segment 320 of the endovascular device ranges from about 20 cm to about 160 cm. According to some embodiments, the working segment 380 of the endovascular device ranges from about 20 cm to at least 160 cm. According to some embodiments, the length of the working segment 380 of the endovascular device is at least 20 cm. According to some embodiments, the length of the working segment 380 of the endovascular device is at least 25 cm. According to some embodiments, the length of the working segment 380 of the endovascular device is at least 30 cm. According to some embodiments, the length of the working segment 380 of the endovascular device is at least 35 cm. According to some embodiments, the length of the working segment 380 of the endovascular device is at least 40 cm. According to some embodiments, the length of the working segment 380 of the endovascular device is at least 45 cm. According to some embodiments, the length of the working segment 380 of the endovascular device is at least 50 cm. According to some embodiments, the length of the working segment 380 of the endovascular device is at least 55 cm. According to some embodiments, the length of the working segment 380 of the endovascular device is at least 60 cm. According to some embodiments, the length of the working segment 380 of the endovascular device is at least 65 cm. According to some embodiments, the length of the working segment 380 of the endovascular device is at least 70 cm. According to some embodiments, the length of the working segment 380 of the endovascular device is at least 75 cm. According to some embodiments, the length of the working segment 380 of the endovascular device is at least 80 cm. According to some embodiments, the length of the working segment 380 of the endovascular device is at least 85 cm. According to some embodiments, the length of the working segment 380 of the endovascular device is at least 90 cm. According to some embodiments, the length of the working segment 380 of the endovascular device is at least 95 cm. According to some embodiments, the length of the working segment 380 of the endovascular device is at least 100 cm. According to some embodiments, the length of the working segment 380 of the endovascular device is at least 105 cm. According to some embodiments, the length of the working segment 380 of the endovascular device is at least 110 cm. According to some embodiments, the length of the working segment 380 of the endovascular device is at least 115 cm. According to some embodiments, the length of the working segment 380 of the endovascular device is at least 120 cm. According to some embodiments, the length of the working segment 380 of the endovascular device is at least 125 cm. According to some embodiments, the length of the working segment 380 of the endovascular device is at least 130 cm. According to some embodiments, the length of the working segment 380 of the endovascular device is at least 135 cm. According to some embodiments, the length of the working segment 380 of the endovascular device is at least 140 cm. According to some embodiments, the working segment 380 of the endovascular device is at least 145 cm. According to some embodiments, the length of the working segment 380 of the endovascular device is at least 150 cm. According to some embodiments, the length of the working segment 380 of the endovascular device is at least 155 cm. According to some embodiments, the length of the working segment 380 of the endovascular device is at least 160 cm.

According to some embodiments, the inner diameter (ID) of the lumen 370 in the working segment of the endovascular device ranges from about 0.1 French (Fr) (0.001 inches) to about 30 French (Fr) (0.394 inches). According to some embodiments, the inner diameter (ID) of the lumen 370 in the of the working segment of the endovascular device ranges from about 0.1 French (Fr) (0.001 inches) to at least 30 French (Fr) (0.394 inches). According to some embodiments, the inner diameter (ID) of the lumen 370 in the of the working segment of the endovascular device ranges from at least 0.1 French (Fr) (0.001 inches) to at least 30 French (Fr) (0.394 inches). According to some embodiments, the inner diameter (ID) of the lumen 370 in the of the working segment of the endovascular device is at least 0.1 French (Fr) (0.001 inches). According to some embodiments, the inner diameter (ID) of the lumen 370 in the of the working segment of the endovascular device is at least 0.2 French (Fr) (0.003 inches). According to some embodiments, the inner diameter (ID) of the lumen 370 in the working segment of the endovascular device is at least 0.3 French (Fr) (0.004 inches). According to some embodiments, the inner diameter (ID) of the lumen 370 in the working segment of the endovascular device is at least 0.4 French (Fr) (0.005 inches). According to some embodiments, the inner diameter (ID) of the lumen 370 in the working segment of the endovascular device is at least 0.5 French (Fr) (0.007 inches). According to some embodiments, the inner diameter (ID) of the lumen 370 in the working segment of the endovascular device is at least 0.6 French (Fr) (0.008 inches). According to some embodiments, the inner diameter (ID) of the lumen 370 in the working segment of the endovascular device is at least 0.7 French (Fr) (0.009 inches). According to some embodiments, the inner diameter (ID) of the lumen 370 in the working segment of the endovascular device is at least 0.8 French (Fr) (0.01 inches). According to some embodiments, the inner diameter (ID) of the lumen 370 in the working segment of the endovascular device is at least 0.9 French (Fr) (0.012 inches). According to some embodiments, the inner diameter (ID) of the lumen 370 in the working segment of the endovascular device is at least 1 French (Fr) (0.013 inches). According to some embodiments, the inner diameter (ID) of the lumen 370 in the working segment of the endovascular device is at least 2 French (Fr) (0.026 inches). According to some embodiments, the inner diameter (ID) of the lumen 370 in the working segment of the endovascular device is at least 3 French (Fr) (0.039 inches). According to some embodiments, the inner diameter (ID) of the lumen 370 in the working segment of the endovascular device is at least 4 French (Fr) (0.052 inches). According to some embodiments, the inner diameter (ID) of the lumen 370 in the working segment of the endovascular device is at least 5 French (Fr) (0.066 inches). According to some embodiments, the inner diameter (ID) of the lumen 370 in the working segment of the endovascular device is at least 6 French (Fr) (0.079 inches). According to some embodiments, the inner diameter (ID) of the lumen 370 in the working segment of the endovascular device is at least 7 French (Fr) (0.092 inches). According to some embodiments, the inner diameter (ID) of the lumen 370 in the working segment of the endovascular device is at least 8 French (Fr) (0.105 inches). According to some embodiments, the inner diameter (ID) of the lumen 370 in the working segment of the endovascular device is at least 9 French (Fr) (0.118 inches). According to some embodiments, the inner diameter (ID) of the lumen 370 in the working segment of the endovascular device is at least 10 French (Fr) (0.131 inches). According to some embodiments, the inner diameter (ID) of the lumen 370 in the working segment of the endovascular device is at least 11 French (Fr) (0.144 inches). According to some embodiments, the inner diameter (ID) of the lumen 370 in the working segment of the endovascular device is at least 12 French (Fr) (0.157 inches). According to some embodiments, the inner diameter (ID) of the lumen 370 in the working segment of the endovascular device is at least 13 French (Fr) (0.171 inches). According to some embodiments, the inner diameter (ID) of the lumen 370 in the working segment of the endovascular device is at least 14 French (Fr) (0.184 inches).). According to some embodiments, the inner diameter (ID) of the lumen 370 in the working segment of the endovascular device is at least 15 French (Fr) (0.197 inches). According to some embodiments, the inner diameter (ID) of the lumen 370 in the working segment of the endovascular device is at least 16 French (Fr) (0.210 inches). According to some embodiments, the inner diameter (ID) of the lumen 370 in the working segment of the endovascular device is at least 17 French (Fr) (0.223 inches). According to some embodiments, the inner diameter (ID) of the lumen 370 in the working segment of the endovascular device is at least 18 French (Fr) (0.236 inches). According to some embodiments, the inner diameter (ID) of the lumen 370 in the working segment of the endovascular device is at least 19 French (Fr) (0.249 inches). According to some embodiments, the inner diameter (ID) of the lumen 370 in the working segment of the endovascular device is at least 20 French (Fr) (0.262 inches). According to some embodiments, the inner diameter (ID) of the lumen 370 in the working segment of the endovascular device is at least 21 French (Fr) (0.276 inches). According to some embodiments, the inner diameter (ID) of the lumen 370 in the working segment of the endovascular device is at least 22 French (Fr) (0.289 inches). According to some embodiments, the inner diameter (ID) of the lumen 370 in the working segment of the endovascular device is at least 23 French (Fr) (0.302 inches). According to some embodiments, the inner diameter (ID) of the lumen 370 in the working segment of the endovascular device is at least 24 French (Fr) (0.315 inches). According to some embodiments, the inner diameter (ID) of the lumen 370 in the working segment of the endovascular device is at least 25 French (Fr) (0.328 inches). According to some embodiments, the inner diameter (ID) of the lumen 370 in the working segment of the endovascular device is at least 26 French (Fr) (0.341 inches). According to some embodiments, the inner diameter (ID) of the lumen 370 in the working segment of the endovascular device is at least 27 French (Fr) (0.354 inches). According to some embodiments, the inner diameter (ID) of the lumen 370 in the working segment of the endovascular device is at least 28 French (Fr) (0.367 inches). According to some embodiments, the inner diameter (ID) of the lumen 370 in the working segment of the endovascular device is at least 29 French (Fr) (0.381 inches). According to some embodiments, the inner diameter (ID) of the lumen 370 in the working segment of the endovascular device is at least 30 French (Fr) (0.394 inches).

According to some embodiments, the length of support segment 390 of endovascular device 300 for intracranial applications ranges from about 0.01% to about 20% of the length of the working segment 380. According to some such embodiments, the length of the support segment 390 of the endovascular device ranges from about 0.01% to at least 20% of the length of the working segment 380. According to some such embodiments, the length of the support segment 390 of the endovascular device is at least 0.1% of the length of the working segment 380. According to some such embodiments, the length of the support segment 390 of the endovascular device is at least 0.2% of the length of the working segment 380. According to some such embodiments, the length of the support segment 390 of the endovascular device is at least 0.3% of the length of the working segment 380. According to some such embodiments, the length of the support segment 390 of the endovascular device is at least 0.4% of the length of the working segment 380. According to some such embodiments, the length of the support segment 390 of the endovascular device is at least 0.5% of the length of the working segment 380. According to some such embodiments, the length of the support segment 390 of the endovascular device is at least 0.6% of the length of the working segment 380. According to some such embodiments, the length of the support segment 390 of the endovascular device is at least 0.7% of the length of the working segment 380. According to some such embodiments, the length of the support segment 390 of the endovascular device is at least 0.8% of the length of the working segment 380. According to some such embodiments, the length of the support segment 390 of the endovascular device is at least 0.9% of the length of the working segment 380. According to some such embodiments, the length of the support segment 390 of the endovascular device is at least 1% of the length of the working segment 380. According to some such embodiments, the length of the support segment 390 of the endovascular device is at least 1.1% of the length of the working segment 380. According to some such embodiments, the length of the support segment 390 of the endovascular device is at least 1.2% of the length of the working segment 380. According to some such embodiments, the length of the support segment 390 of the endovascular device is at least 1.3% of the length of the working segment 380. According to some such embodiments, the length of the support segment 390 of the endovascular device is at least 1.4% of the length of the working segment 380. According to some such embodiments, the length of the support segment 390 of the endovascular device is at least 1.5% of the length of the working segment 380. According to some such embodiments, the length of the support segment 390 of the endovascular device is at least 1.6% of the length of the working segment 380. According to some such embodiments, the length of the support segment 390 of the endovascular device is at least 1.7% of the length of the working segment 380. According to some such embodiments, the length of the support segment 390 of the endovascular device is at least 1.8% of the length of the working segment 380. According to some such embodiments, the length of the support segment 390 of the endovascular device is at least 1.9% of the length of the working segment 380. According to some such embodiments, the length of the support segment 390 of the endovascular device is at least 2% of the length of the working segment 380. According to some such embodiments, the length of the support segment 390 of the endovascular device is at least 2.1% of the length of the working segment 380. According to some such embodiments, the length of the support segment 390 of the endovascular device is at least 2.2% of the length of the working segment 380. According to some such embodiments, the length of the support segment 390 of the endovascular device is at least 2.3% of the length of the working segment 380. According to some such embodiments, the length of the support segment 390 of the endovascular device is at least 2.4% of the length of the working segment 380. According to some such embodiments, the length of the support segment 390 of the endovascular device is at least 2.5% of the length of the working segment 380. According to some such embodiments, the length of the support segment 390 of the endovascular device is at least 2.6% of the length of the working segment 380. According to some such embodiments, the length of the support segment 390 of the endovascular device is at least 2.7% of the length of the working segment 380. According to some such embodiments, the length of the support segment 390 of the endovascular device is at least 2.8% of the length of the working segment 380. According to some such embodiments, the length of the support segment 390 of the endovascular device is at least 2.9% of the length of the working segment 380. According to some such embodiments, the length of the support segment 390 of the endovascular device is at least 3% of the length of the working segment 380. According to some such embodiments, the length of the support segment 390 of the endovascular device is at least 3.1% of the length of the working segment 380. According to some such embodiments, the length of the support segment 390 of the endovascular device is at least 3.2% of the length of the working segment 380. According to some such embodiments, the length of the support segment 390 of the endovascular device is at least 3.3% of the length of the working segment 380. According to some such embodiments, the length of the support segment 390 of the endovascular device is at least 3.4% of the length of the working segment 380. According to some such embodiments, the length of the support segment 390 of the endovascular device is at least 3.5% of the length of the working segment 380. According to some such embodiments, the length of the support segment 390 of the endovascular device is at least 3.6% of the length of the working segment 380. According to some such embodiments, the length of the support segment 390 of the endovascular device is at least 3.7% of the length of the working segment 380. According to some such embodiments, the length of the support segment 390 of the endovascular device is at least 3.8% of the length of the working segment 380. According to some such embodiments, the length of the support segment 390 of the endovascular device is at least 3.9% of the length of the working segment 380. According to some such embodiments, the length of the support segment 390 of the endovascular device is at least 4% of the length of the working segment 380. According to some such embodiments, the length of the support segment 390 of the endovascular device is at least 4.1% of the length of the working segment 380. According to some such embodiments, the length of the support segment 390 of the endovascular device is at least 4.2% of the length of the working segment 380. According to some such embodiments, the length of the support segment 390 of the endovascular device is at least 4.3% of the length of the working segment 380. According to some such embodiments, the length of the support segment 390 of the endovascular device is at least 4.4% of the length of the working segment 380. According to some such embodiments, the length of the support segment 390 of the endovascular device is at least 4.5% of the length of the working segment 380. According to some such embodiments, the length of the support segment 390 of the endovascular device is at least 4.6% of the length of the working segment 380. According to some such embodiments, the length of the support segment 390 of the endovascular device is at least 4.7% of the length of the working segment 380. According to some such embodiments, the length of the support segment 390 of the endovascular device is at least 4.8% of the length of the working segment 380. According to some such embodiments, the length of the support segment 390 of the endovascular device is at least 4.9% of the length of the working segment 380. According to some such embodiments, the length of the support segment 390 of the endovascular device is at least 5% of the length of the working segment 380. According to some such embodiments, the length of the support segment 390 of the endovascular device is at least 5.1% of the length of the working segment 380. According to some such embodiments, the length of the support segment 390 of the endovascular device is at least 5.2% of the length of the working segment 380. According to some such embodiments, the length of the support segment 390 of the endovascular device is at least 5.3% of the length of the working segment 380. According to some such embodiments, the length of the support segment 390 of the endovascular device is at least 5.4% of the length of the working segment 380. According to some such embodiments, the length of the support segment 390 of the endovascular device is at least 5.5% of the length of the working segment 380. According to some such embodiments, the length of the support segment 390 of the endovascular device is at least 5.6% of the length of the working segment 380. According to some such embodiments, the length of the support segment 390 of the endovascular device is at least 5.7% of the length of the working segment 380. According to some such embodiments, the length of the support segment 390 of the endovascular device is at least 5.8% of the length of the working segment 380. According to some such embodiments, the length of the support segment 390 of the endovascular device is at least 5.9% of the length of the working segment 380. According to some such embodiments, the length of the support segment 390 of the endovascular device is at least 6% of the length of the working segment 380. According to some such embodiments, the length of the support segment 390 of the endovascular device is at least 6.1% of the length of the working segment 380. According to some such embodiments, the length of the support segment 390 of the endovascular device is at least 6.2% of the length of the working segment 380. According to some such embodiments, the length of the support segment 390 of the endovascular device is at least 6.3% of the length of the working segment 380. According to some such embodiments, the length of the support segment 390 of the endovascular device is at least 6.4% of the length of the working segment 380. According to some such embodiments, the length of the support segment 390 of the endovascular device is at least 6.5% of the length of the working segment 380. According to some such embodiments, the length of the support segment 390 of the endovascular device is at least 6.6% of the length of the working segment 380. According to some such embodiments, the length of the support segment 390 of the endovascular device is at least 6.7% of the length of the working segment 380. According to some such embodiments, the length of the support segment 390 of the endovascular device is at least 6.8% of the length of the working segment 380. According to some such embodiments, the length of the support segment 390 of the endovascular device is at least 6.9% of the length of the working segment 380. According to some such embodiments, the length of the support segment 390 of the endovascular device is at least 7% of the length of the working segment 380. According to some such embodiments, the length of the support segment 390 of the endovascular device is at least 7.1% of the length of the working segment 380. According to some such embodiments, the length of the support segment 390 of the endovascular device is at least 7.2% of the length of the working segment 380. According to some such embodiments, the length of the support segment 390 of the endovascular device is at least 7.3% of the length of the working segment 380. According to some such embodiments, the length of the support segment 390 of the endovascular device is at least 7.4% of the length of the working segment 380. According to some such embodiments, the length of the support segment 390 of the endovascular device is at least 7.5% of the length of the working segment 380. According to some such embodiments, the length of the support segment 390 of the endovascular device is at least 7.6% of the length of the working segment 380. According to some such embodiments, the length of the support segment 390 of the endovascular device is at least 7.7% of the length of the working segment 380. According to some such embodiments, the length of the support segment 390 of the endovascular device is at least 7.8% of the length of the working segment 380. According to some such embodiments, the length of the support segment 390 of the endovascular device is at least 7.9% of the length of the working segment 380. According to some such embodiments, the length of the support segment 390 of the endovascular device is at least 8% of the length of the working segment 380. According to some such embodiments, the length of the support segment 390 of the endovascular device is at least 8.1% of the length of the working segment 380. According to some such embodiments, the length of the support segment 390 of the endovascular device is at least 8.2% of the length of the working segment 380. According to some such embodiments, the length of the support segment 390 of the endovascular device is at least 8.3% of the length of the working segment 380. According to some such embodiments, the length of the support segment 390 of the endovascular device is at least 8.4% of the length of the working segment 380. According to some such embodiments, the length of the support segment 390 of the endovascular device is at least 8.5% of the length of the working segment 380. According to some such embodiments, the length of the support segment 390 of the endovascular device is at least 8.6% of the length of the working segment 380. According to some such embodiments, the length of the support segment 390 of the endovascular device is at least 8.7% of the length of the working segment 380. According to some such embodiments, the length of the support segment 390 of the endovascular device is at least 8.8% of the length of the working segment 380. According to some such embodiments, the length of the support segment 390 of the endovascular device is at least 8.9% of the length of the working segment 380. According to some such embodiments, the length of the support segment 390 of the endovascular device is at least 9% of the length of the working segment 380. According to some such embodiments, the length of the support segment 390 of the endovascular device is at least 9.1% of the length of the working segment 380. According to some such embodiments, the length of the support segment 390 of the endovascular device is at least 9.2% of the length of the working segment 380. According to some such embodiments, the length of the support segment 390 of the endovascular device is at least 9.3% of the length of the working segment 380. According to some such embodiments, the length of the support segment 390 of the endovascular device is at least 9.4% of the length of the working segment 380. According to some such embodiments, the length of the support segment 390 of the endovascular device is at least 9.5% of the length of the working segment 380. According to some such embodiments, the length of the support segment 390 of the endovascular device is at least 9.6% of the length of the working segment 380. According to some such embodiments, the length of the support segment 390 of the endovascular device is at least 9.7% of the length of the working segment 380. According to some such embodiments, the length of the support segment 390 of the endovascular device is at least 9.8% of the length of the working segment 380. According to some such embodiments, the length of the support segment 390 of the endovascular device is at least 9.9% of the length of the working segment 380. According to some such embodiments, the length of the support segment 390 of the endovascular device is at least 10% of the length of the working segment 380.

According to some such embodiments, the length of the support segment 390 of the endovascular device is at least 10.1% of the length of the working segment 380. According to some such embodiments, the length of the support segment 390 of the endovascular device is at least 10.2% of the length of the working segment 380. According to some such embodiments, the length of the support segment 390 of the endovascular device is at least 10.3% of the length of the working segment 380. According to some such embodiments, the length of the support segment 390 of the endovascular device is at least 10.4% of the length of the working segment 380. According to some such embodiments, the length of the support segment 390 of the endovascular device is at least 10.5% of the length of the working segment 380. According to some such embodiments, the length of the support segment 390 of the endovascular device is at least 10.6% of the length of the working segment 380. According to some such embodiments, the length of the support segment 390 of the endovascular device is at least 10.7% of the length of the working segment 380. According to some such embodiments, the length of the support segment 390 of the endovascular device is at least 10.8% of the length of the working segment 380. According to some such embodiments, the length of the support segment 390 of the endovascular device is at least 10.9% of the length of the working segment 380. According to some such embodiments, the length of the support segment 390 of the endovascular device is at least 11% of the length of the working segment 380.

According to some such embodiments, the length of the support segment 390 of the endovascular device is at least 11.1% of the length of the working segment 380. According to some such embodiments, the length of the support segment 390 of the endovascular device is at least 11.2% of the length of the working segment 380. According to some such embodiments, the length of the support segment 390 of the endovascular device is at least 11.3% of the length of the working segment 380. According to some such embodiments, the length of the support segment 390 of the endovascular device is at least 11.4% of the length of the working segment 380. According to some such embodiments, the length of the support segment 390 of the endovascular device is at least 11.5% of the length of the working segment 380. According to some such embodiments, the length of the support segment 390 of the endovascular device is at least 11.6% of the length of the working segment 380. According to some such embodiments, the length of the support segment 390 of the endovascular device is at least 11.7% of the length of the working segment 380. According to some such embodiments, the length of the support segment 390 of the endovascular device is at least 11.8% of the length of the working segment 380. According to some such embodiments, the length of the support segment 390 of the endovascular device is at least 11.9% of the length of the working segment 380. According to some such embodiments, the length of the support segment 390 of the endovascular device is at least 12% of the length of the working segment 380.

According to some such embodiments, the length of the support segment 390 of the endovascular device is at least 12.1% of the length of the working segment 380. According to some such embodiments, the length of the support segment 390 of the endovascular device is at least 12.2% of the length of the working segment 380. According to some such embodiments, the length of the support segment 390 of the endovascular device is at least 12.3% of the length of the working segment 380. According to some such embodiments, the length of the support segment 390 of the endovascular device is at least 12.4% of the length of the working segment 380. According to some such embodiments, the length of the support segment 390 of the endovascular device is at least 12.5% of the length of the working segment 380. According to some such embodiments, the length of the support segment 390 of the endovascular device is at least 12.6% of the length of the working segment 380. According to some such embodiments, the length of the support segment 390 of the endovascular device is at least 12.7% of the length of the working segment 380. According to some such embodiments, the length of the support segment 390 of the endovascular device is at least 12.8% of the length of the working segment 380. According to some such embodiments, the length of the support segment 390 of the endovascular device is at least 12.9% of the length of the working segment 380. According to some such embodiments, the length of the support segment 390 of the endovascular device is at least 13% of the length of the working segment 380.

According to some such embodiments, the length of the support segment 390 of the endovascular device is at least 13.1% of the length of the working segment 380. According to some such embodiments, the length of the support segment 390 of the endovascular device is at least 13.2% of the length of the working segment 380. According to some such embodiments, the length of the support segment 390 of the endovascular device is at least 13.3% of the length of the working segment 380. According to some such embodiments, the length of the support segment 390 of the endovascular device is at least 13.4% of the length of the working segment 380. According to some such embodiments, the length of the support segment 390 of the endovascular device is at least 13.5% of the length of the working segment 380. According to some such embodiments, the length of the support segment 390 of the endovascular device is at least 13.6% of the length of the working segment 380. According to some such embodiments, the length of the support segment 390 of the endovascular device is at least 13.7% of the length of the working segment 380. According to some such embodiments, the length of the support segment 390 of the endovascular device is at least 13.8% of the length of the working segment 380. According to some such embodiments, the length of the support segment 390 of the endovascular device is at least 13.9% of the length of the working segment 380. According to some such embodiments, the length of the support segment 390 of the endovascular device is at least 14% of the length of the working segment 380.

According to some such embodiments, the length of the support segment 390 of the endovascular device is at least 14.1% of the length of the working segment 380. According to some such embodiments, the length of the support segment 390 of the endovascular device is at least 14.2% of the length of the working segment 380. According to some such embodiments, the length of the support segment 390 of the endovascular device is at least 14.3% of the length of the working segment 380. According to some such embodiments, the length of the support segment 390 of the endovascular device is at least 14.4% of the length of the working segment 380. According to some such embodiments, the length of the support segment 390 of the endovascular device is at least 14.5% of the length of the working segment 380. According to some such embodiments, the length of the support segment 390 of the endovascular device is at least 14.6% of the length of the working segment 380. According to some such embodiments, the length of the support segment 390 of the endovascular device is at least 14.7% of the length of the working segment 380. According to some such embodiments, the length of the support segment 390 of the endovascular device is at least 14.8% of the length of the working segment 380. According to some such embodiments, the length of the support segment 390 of the endovascular device is at least 14.9% of the length of the working segment 380. According to some such embodiments, the length of the support segment 390 of the endovascular device is at least 15% of the length of the working segment 380.

According to some such embodiments, the length of the support segment 390 of the endovascular device is at least 15.1% of the length of the working segment 380. According to some such embodiments, the length of the support segment 390 of the endovascular device is at least 15.2% of the length of the working segment 380. According to some such embodiments, the length of the support segment 390 of the endovascular device is at least 15.3% of the length of the working segment 380. According to some such embodiments, the length of the support segment 390 of the endovascular device is at least 15.4% of the length of the working segment 380. According to some such embodiments, the length of the support segment 390 of the endovascular device is at least 15.5% of the length of the working segment 380. According to some such embodiments, the length of the support segment 390 of the endovascular device is at least 15.6% of the length of the working segment 380. According to some such embodiments, the length of the support segment 390 of the endovascular device is at least 15.7% of the length of the working segment 380. According to some such embodiments, the length of the support segment 390 of the endovascular device is at least 15.8% of the length of the working segment 380. According to some such embodiments, the length of the support segment 390 of the endovascular device is at least 15.9% of the length of the working segment 380. According to some such embodiments, the length of the support segment 390 of the endovascular device is at least 16% of the length of the working segment 380.

According to some such embodiments, the length of the support segment 390 of the endovascular device is at least 16.1% of the length of the working segment 380. According to some such embodiments, the length of the support segment 390 of the endovascular device is at least 16.2% of the length of the working segment 380. According to some such embodiments, the length of the support segment 390 of the endovascular device is at least 16.3% of the length of the working segment 380. According to some such embodiments, the length of the support segment 390 of the endovascular device is at least 16.4% of the length of the working segment 380. According to some such embodiments, the length of the support segment 390 of the endovascular device is at least 16.5% of the length of the working segment 380. According to some such embodiments, the length of the support segment 390 of the endovascular device is at least 16.6% of the length of the working segment 380. According to some such embodiments, the length of the support segment 390 of the endovascular device is at least 16.7% of the length of the working segment 380. According to some such embodiments, the length of the support segment 390 of the endovascular device is at least 16.8% of the length of the working segment 380. According to some such embodiments, the length of the support segment 390 of the endovascular device is at least 16.9% of the length of the working segment 380. According to some such embodiments, the length of the support segment 390 of the endovascular device is at least 17% of the length of the working segment 380.

According to some such embodiments, the length of the support segment 390 of the endovascular device is at least 17.1% of the length of the working segment 380. According to some such embodiments, the length of the support segment 390 of the endovascular device is at least 17.2% of the length of the working segment 380. According to some such embodiments, the length of the support segment 390 of the endovascular device is at least 17.3% of the length of the working segment 380. According to some such embodiments, the length of the support segment 390 of the endovascular device is at least 17.4% of the length of the working segment 380. According to some such embodiments, the length of the support segment 390 of the endovascular device is at least 17.5% of the length of the working segment 380. According to some such embodiments, the length of the support segment 390 of the endovascular device is at least 17.6% of the length of the working segment 380. According to some such embodiments, the length of the support segment 390 of the endovascular device is at least 17.7% of the length of the working segment 380. According to some such embodiments, the length of the support segment 390 of the endovascular device is at least 17.8% of the length of the working segment 380. According to some such embodiments, the length of the support segment 390 of the endovascular device is at least 17.9% of the length of the working segment 380. According to some such embodiments, the length of the support segment 390 of the endovascular device is at least 18% of the length of the working segment 380.

According to some such embodiments, the length of the support segment 390 of the endovascular device is at least 18.1% of the length of the working segment 380. According to some such embodiments, the length of the support segment 390 of the endovascular device is at least 18.2% of the length of the working segment 380. According to some such embodiments, the length of the support segment 390 of the endovascular device is at least 18.3% of the length of the working segment 380. According to some such embodiments, the length of the support segment 390 of the endovascular device is at least 18.4% of the length of the working segment 380. According to some such embodiments, the length of the support segment 390 of the endovascular device is at least 18.5% of the length of the working segment 380. According to some such embodiments, the length of the support segment 390 of the endovascular device is at least 18.6% of the length of the working segment 380. According to some such embodiments, the length of the support segment 390 of the endovascular device is at least 18.7% of the length of the working segment 380. According to some such embodiments, the length of the support segment 390 of the endovascular device is at least 18.8% of the length of the working segment 380. According to some such embodiments, the length of the support segment 390 of the endovascular device is at least 18.9% of the length of the working segment 380. According to some such embodiments, the length of the support segment 390 of the endovascular device is at least 19% of the length of the working segment 380.

According to some such embodiments, the length of the support segment 390 of the endovascular device is at least 19.1% of the length of the working segment 380. According to some such embodiments, the length of the support segment 390 of the endovascular device is at least 19.2% of the length of the working segment 380. According to some such embodiments, the length of the support segment 390 of the endovascular device is at least 19.3% of the length of the working segment 380. According to some such embodiments, the length of the support segment 390 of the endovascular device is at least 19.4% of the length of the working segment 380. According to some such embodiments, the length of the support segment 390 of the endovascular device is at least 19.5% of the length of the working segment 380. According to some such embodiments, the length of the support segment 390 of the endovascular device is at least 19.6% of the length of the working segment 380. According to some such embodiments, the length of the support segment 390 of the endovascular device is at least 19.7% of the length of the working segment 380. According to some such embodiments, the length of the support segment 390 of the endovascular device is at least 19.8% of the length of the working segment 380. According to some such embodiments, the length of the support segment 390 of the endovascular device is at least 19.9% of the length of the working segment 380. According to some such embodiments, the length of the support segment 390 of the endovascular device is at least 20% of the length of the working segment 380.

According to some such embodiments, the length of the support segment 390 of the endovascular device for peripheral applications ranges from about 0.01% to at least 200% of the length of the working segment 380. According to some such embodiments, the length of the support segment 390 of the endovascular device is at least 0.1% of the length of the working segment 380. According to some such embodiments, the length of the support segment 390 of the endovascular device is at least 0.2% of the length of the working segment 380. According to some such embodiments, the length of the support segment 390 of the endovascular device is at least 0.3% of the length of the working segment 380. According to some such embodiments, the length of the support segment 390 of the endovascular device is at least 0.4% of the length of the working segment 380. According to some such embodiments, the length of the support segment 390 of the endovascular device is at least 0.5% of the length of the working segment 380. According to some such embodiments, the length of the support segment 390 of the endovascular device is at least 0.6% of the length of the working segment 380. According to some such embodiments, the length of the support segment 390 of the endovascular device is at least 0.7% of the length of the working segment 380. According to some such embodiments, the length of the support segment 390 of the endovascular device is at least 0.8% of the length of the working segment 380. According to some such embodiments, the length of the support segment 390 of the endovascular device is at least 0.9% of the length of the working segment 380. According to some such embodiments, the length of the support segment 390 of the endovascular device is at least 1% of the length of the working segment 380. According to some such embodiments, the length of the support segment 390 of the endovascular device is at least 1.1% of the length of the working segment 380. According to some such embodiments, the length of the support segment 390 of the endovascular device is at least 1.2% of the length of the working segment 380. According to some such embodiments, the length of the support segment 390 of the endovascular device is at least 1.3% of the length of the working segment 380. According to some such embodiments, the length of the support segment 390 of the endovascular device is at least 1.4% of the length of the working segment 380. According to some such embodiments, the length of the support segment 390 of the endovascular device is at least 1.5% of the length of the working segment 380. According to some such embodiments, the length of the support segment 390 of the endovascular device is at least 1.6% of the length of the working segment 380. According to some such embodiments, the length of the support segment 390 of the endovascular device is at least 1.7% of the length of the working segment 380. According to some such embodiments, the length of the support segment 390 of the endovascular device is at least 1.8% of the length of the working segment 380. According to some such embodiments, the length of the support segment 390 of the endovascular device is at least 1.9% of the length of the working segment 380. According to some such embodiments, the length of the support segment 390 of the endovascular device is at least 2% of the length of the working segment 380. According to some such embodiments, the length of the support segment 390 of the endovascular device is at least 2.1% of the length of the working segment 380. According to some such embodiments, the length of the support segment 390 of the endovascular device is at least 2.2% of the length of the working segment 380. According to some such embodiments, the length of the support segment 390 of the endovascular device is at least 2.3% of the length of the working segment 380. According to some such embodiments, the length of the support segment 390 of the endovascular device is at least 2.4% of the length of the working segment 380. According to some such embodiments, the length of the support segment 390 of the endovascular device is at least 2.5% of the length of the working segment 380. According to some such embodiments, the length of the support segment 390 of the endovascular device is at least 2.6% of the length of the working segment 380. According to some such embodiments, the length of the support segment 390 of the endovascular device is at least 2.7% of the length of the working segment 380. According to some such embodiments, the length of the support segment 390 of the endovascular device is at least 2.8% of the length of the working segment 380. According to some such embodiments, the length of the support segment 390 of the endovascular device is at least 2.9% of the length of the working segment 380. According to some such embodiments, the length of the support segment 390 of the endovascular device is at least 3% of the length of the working segment 380. According to some such embodiments, the length of the support segment 390 of the endovascular device is at least 3.1% of the length of the working segment 380. According to some such embodiments, the length of the support segment 390 of the endovascular device is at least 3.2% of the length of the working segment 380. According to some such embodiments, the length of the support segment 390 of the endovascular device is at least 3.3% of the length of the working segment 380. According to some such embodiments, the length of the support segment 390 of the endovascular device is at least 3.4% of the length of the working segment 380. According to some such embodiments, the length of the support segment 390 of the endovascular device is at least 3.5% of the length of the working segment 380. According to some such embodiments, the length of the support segment 390 of the endovascular device is at least 3.6% of the length of the working segment 380. According to some such embodiments, the length of the support segment 390 of the endovascular device is at least 3.7% of the length of the working segment 380. According to some such embodiments, the length of the support segment 390 of the endovascular device is at least 3.8% of the length of the working segment 380. According to some such embodiments, the length of the support segment 390 of the endovascular device is at least 3.9% of the length of the working segment 380. According to some such embodiments, the length of the support segment 390 of the endovascular device is at least 4% of the length of the working segment 380. According to some such embodiments, the length of the support segment 390 of the endovascular device is at least 4.1% of the length of the working segment 380. According to some such embodiments, the length of the support segment 390 of the endovascular device is at least 4.2% of the length of the working segment 380. According to some such embodiments, the length of the support segment 390 of the endovascular device is at least 4.3% of the length of the working segment 380. According to some such embodiments, the length of the support segment 390 of the endovascular device is at least 4.4% of the length of the working segment 380. According to some such embodiments, the length of the support segment 390 of the endovascular device is at least 4.5% of the length of the working segment 380. According to some such embodiments, the length of the support segment 390 of the endovascular device is at least 4.6% of the length of the working segment 380. According to some such embodiments, the length of the support segment 390 of the endovascular device is at least 4.7% of the length of the working segment 380. According to some such embodiments, the length of the support segment 390 of the endovascular device is at least 4.8% of the length of the working segment 380. According to some such embodiments, the length of the support segment 390 of the endovascular device is at least 4.9% of the length of the working segment 380. According to some such embodiments, the length of the support segment 390 of the endovascular device is at least 5% of the length of the working segment 380. According to some such embodiments, the length of the support segment 390 of the endovascular device is at least 5.1% of the length of the working segment 380. According to some such embodiments, the length of the support segment 390 of the endovascular device is at least 5.2% of the length of the working segment 380. According to some such embodiments, the length of the support segment 390 of the endovascular device is at least 5.3% of the length of the working segment 380. According to some such embodiments, the length of the support segment 390 of the endovascular device is at least 5.4% of the length of the working segment 380. According to some such embodiments, the length of the support segment 390 of the endovascular device is at least 5.5% of the length of the working segment 380. According to some such embodiments, the length of the support segment 390 of the endovascular device is at least 5.6% of the length of the working segment 380. According to some such embodiments, the length of the support segment 390 of the endovascular device is at least 5.7% of the length of the working segment 380. According to some such embodiments, the length of the support segment 390 of the endovascular device is at least 5.8% of the length of the working segment 380. According to some such embodiments, the length of the support segment 390 of the endovascular device is at least 5.9% of the length of the working segment 380. According to some such embodiments, the length of the support segment 390 of the endovascular device is at least 6% of the length of the working segment 380. According to some such embodiments, the length of the support segment 390 of the endovascular device is at least 6.1% of the length of the working segment 380. According to some such embodiments, the length of the support segment 390 of the endovascular device is at least 6.2% of the length of the working segment 380. According to some such embodiments, the length of the support segment 390 of the endovascular device is at least 6.3% of the length of the working segment 380. According to some such embodiments, the length of the support segment 390 of the endovascular device is at least 6.4% of the length of the working segment 380. According to some such embodiments, the length of the support segment 390 of the endovascular device is at least 6.5% of the length of the working segment 380. According to some such embodiments, the length of the support segment 390 of the endovascular device is at least 6.6% of the length of the working segment 380. According to some such embodiments, the length of the support segment 390 of the endovascular device is at least 6.7% of the length of the working segment 380. According to some such embodiments, the length of the support segment 390 of the endovascular device is at least 6.8% of the length of the working segment 380. According to some such embodiments, the length of the support segment 390 of the endovascular device is at least 6.9% of the length of the working segment 380. According to some such embodiments, the length of the support segment 390 of the endovascular device is at least 7% of the length of the working segment 380. According to some such embodiments, the length of the support segment 390 of the endovascular device is at least 7.1% of the length of the working segment 380. According to some such embodiments, the length of the support segment 390 of the endovascular device is at least 7.2% of the length of the working segment 380. According to some such embodiments, the length of the support segment 390 of the endovascular device is at least 7.3% of the length of the working segment 380. According to some such embodiments, the length of the support segment 390 of the endovascular device is at least 7.4% of the length of the working segment 380. According to some such embodiments, the length of the support segment 390 of the endovascular device is at least 7.5% of the length of the working segment 380. According to some such embodiments, the length of the support segment 390 of the endovascular device is at least 7.6% of the length of the working segment 380. According to some such embodiments, the length of the support segment 390 of the endovascular device is at least 7.7% of the length of the working segment 380. According to some such embodiments, the length of the support segment 390 of the endovascular device is at least 7.8% of the length of the working segment 380. According to some such embodiments, the length of the support segment 390 of the endovascular device is at least 7.9% of the length of the working segment 380. According to some such embodiments, the length of the support segment 390 of the endovascular device is at least 8% of the length of the working segment 380. According to some such embodiments, the length of the support segment 390 of the endovascular device is at least 8.1% of the length of the working segment 380. According to some such embodiments, the length of the support segment 390 of the endovascular device is at least 8.2% of the length of the working segment 380. According to some such embodiments, the length of the support segment 390 of the endovascular device is at least 8.3% of the length of the working segment 380. According to some such embodiments, the length of the support segment 390 of the endovascular device is at least 8.4% of the length of the working segment 380. According to some such embodiments, the length of the support segment 390 of the endovascular device is at least 8.5% of the length of the working segment 380. According to some such embodiments, the length of the support segment 390 of the endovascular device is at least 8.6% of the length of the working segment 380. According to some such embodiments, the length of the support segment 390 of the endovascular device is at least 8.7% of the length of the working segment 380. According to some such embodiments, the length of the support segment 390 of the endovascular device is at least 8.8% of the length of the working segment 380. According to some such embodiments, the length of the support segment 390 of the endovascular device is at least 8.9% of the length of the working segment 380. According to some such embodiments, the length of the support segment 390 of the endovascular device is at least 9% of the length of the working segment 380. According to some such embodiments, the length of the support segment 390 of the endovascular device is at least 9.1% of the length of the working segment 380. According to some such embodiments, the length of the support segment 390 of the endovascular device is at least 9.2% of the length of the working segment 380. According to some such embodiments, the length of the support segment 390 of the endovascular device is at least 9.3% of the length of the working segment 380. According to some such embodiments, the length of the support segment 390 of the endovascular device is at least 9.4% of the length of the working segment 380. According to some such embodiments, the length of the support segment 390 of the endovascular device is at least 9.5% of the length of the working segment 380. According to some such embodiments, the length of the support segment 390 of the endovascular device is at least 9.6% of the length of the working segment 380. According to some such embodiments, the length of the support segment 390 of the endovascular device is at least 9.7% of the length of the working segment 380. According to some such embodiments, the length of the support segment 390 of the endovascular device is at least 9.8% of the length of the working segment 380. According to some such embodiments, the length of the support segment 390 of the endovascular device is at least 9.9% of the length of the working segment 380. According to some such embodiments, the length of the support segment 390 of the endovascular device is at least 10% of the length of the working segment 380.

According to some such embodiments, the length of the support segment 390 of the endovascular device is at least 10.1% of the length of the working segment 380. According to some such embodiments, the length of the support segment 390 of the endovascular device is at least 10.2% of the length of the working segment 380. According to some such embodiments, the length of the support segment 390 of the endovascular device is at least 10.3% of the length of the working segment 380. According to some such embodiments, the length of the support segment 390 of the endovascular device is at least 10.4% of the length of the working segment 380. According to some such embodiments, the length of the support segment 390 of the endovascular device is at least 10.5% of the length of the working segment 380. According to some such embodiments, the length of the support segment 390 of the endovascular device is at least 10.6% of the length of the working segment 380. According to some such embodiments, the length of the support segment 390 of the endovascular device is at least 10.7% of the length of the working segment 380. According to some such embodiments, the length of the support segment 390 of the endovascular device is at least 10.8% of the length of the working segment 380. According to some such embodiments, the length of the support segment 390 of the endovascular device is at least 10.9% of the length of the working segment 380. According to some such embodiments, the length of the support segment 390 of the endovascular device is at least 11% of the length of the working segment 380.

According to some such embodiments, the length of the support segment 390 of the endovascular device is at least 11.1% of the length of the working segment 380. According to some such embodiments, the length of the support segment 390 of the endovascular device is at least 11.2% of the length of the working segment 380. According to some such embodiments, the length of the support segment 390 of the endovascular device is at least 11.3% of the length of the working segment 380. According to some such embodiments, the length of the support segment 390 of the endovascular device is at least 11.4% of the length of the working segment 380. According to some such embodiments, the length of the support segment 390 of the endovascular device is at least 11.5% of the length of the working segment 380. According to some such embodiments, the length of the support segment 390 of the endovascular device is at least 11.6% of the length of the working segment 380. According to some such embodiments, the length of the support segment 390 of the endovascular device is at least 11.7% of the length of the working segment 380. According to some such embodiments, the length of the support segment 390 of the endovascular device is at least 11.8% of the length of the working segment 380. According to some such embodiments, the length of the support segment 390 of the endovascular device is at least 11.9% of the length of the working segment 380. According to some such embodiments, the length of the support segment 390 of the endovascular device is at least 12% of the length of the working segment 380.

According to some such embodiments, the length of the support segment 390 of the endovascular device is at least 12.1% of the length of the working segment 380. According to some such embodiments, the length of the support segment 390 of the endovascular device is at least 12.2% of the length of the working segment 380. According to some such embodiments, the length of the support segment 390 of the endovascular device is at least 12.3% of the length of the working segment 380. According to some such embodiments, the length of the support segment 390 of the endovascular device is at least 12.4% of the length of the working segment 380. According to some such embodiments, the length of the support segment 390 of the endovascular device is at least 12.5% of the length of the working segment 380. According to some such embodiments, the length of the support segment 390 of the endovascular device is at least 12.6% of the length of the working segment 380. According to some such embodiments, the length of the support segment 390 of the endovascular device is at least 12.7% of the length of the working segment 380. According to some such embodiments, the length of the support segment 390 of the endovascular device is at least 12.8% of the length of the working segment 380. According to some such embodiments, the length of the support segment 390 of the endovascular device is at least 12.9% of the length of the working segment 380. According to some such embodiments, the length of the support segment 390 of the endovascular device is at least 13% of the length of the working segment 380.

According to some such embodiments, the length of the support segment 390 of the endovascular device is at least 13.1% of the length of the working segment 380. According to some such embodiments, the length of the support segment 390 of the endovascular device is at least 13.2% of the length of the working segment 380. According to some such embodiments, the length of the support segment 390 of the endovascular device is at least 13.3% of the length of the working segment 380. According to some such embodiments, the length of the support segment 390 of the endovascular device is at least 13.4% of the length of the working segment 380. According to some such embodiments, the length of the support segment 390 of the endovascular device is at least 13.5% of the length of the working segment 380. According to some such embodiments, the length of the support segment 390 of the endovascular device is at least 13.6% of the length of the working segment 380. According to some such embodiments, the length of the support segment 390 of the endovascular device is at least 13.7% of the length of the working segment 380. According to some such embodiments, the length of the support segment 390 of the endovascular device is at least 13.8% of the length of the working segment 380. According to some such embodiments, the length of the support segment 390 of the endovascular device is at least 13.9% of the length of the working segment 380. According to some such embodiments, the length of the support segment 390 of the endovascular device is at least 14% of the length of the working segment 380.

According to some such embodiments, the length of the support segment 390 of the endovascular device is at least 14.1% of the length of the working segment 380. According to some such embodiments, the length of the support segment 390 of the endovascular device is at least 14.2% of the length of the working segment 380. According to some such embodiments, the length of the support segment 390 of the endovascular device is at least 14.3% of the length of the working segment 380. According to some such embodiments, the length of the support segment 390 of the endovascular device is at least 14.4% of the length of the working segment 380. According to some such embodiments, the length of the support segment 390 of the endovascular device is at least 14.5% of the length of the working segment 380. According to some such embodiments, the length of the support segment 390 of the endovascular device is at least 14.6% of the length of the working segment 380. According to some such embodiments, the length of the support segment 390 of the endovascular device is at least 14.7% of the length of the working segment 380. According to some such embodiments, the length of the support segment 390 of the endovascular device is at least 14.8% of the length of the working segment 380. According to some such embodiments, the length of the support segment 390 of the endovascular device is at least 14.9% of the length of the working segment 380. According to some such embodiments, the length of the support segment 390 of the endovascular device is at least 15% of the length of the working segment 380.

According to some such embodiments, the length of the support segment 390 of the endovascular device is at least 15.1% of the length of the working segment 380. According to some such embodiments, the length of the support segment 390 of the endovascular device is at least 15.2% of the length of the working segment 380. According to some such embodiments, the length of the support segment 390 of the endovascular device is at least 15.3% of the length of the working segment 380. According to some such embodiments, the length of the support segment 390 of the endovascular device is at least 15.4% of the length of the working segment 380. According to some such embodiments, the length of the support segment 390 of the endovascular device is at least 15.5% of the length of the working segment 380. According to some such embodiments, the length of the support segment 390 of the endovascular device is at least 15.6% of the length of the working segment 380. According to some such embodiments, the length of the support segment 390 of the endovascular device is at least 15.7% of the length of the working segment 380. According to some such embodiments, the length of the support segment 390 of the endovascular device is at least 15.8% of the length of the working segment 380. According to some such embodiments, the length of the support segment 390 of the endovascular device is at least 15.9% of the length of the working segment 380. According to some such embodiments, the length of the support segment 390 of the endovascular device is at least 16% of the length of the working segment 380.

According to some such embodiments, the length of the support segment 390 of the endovascular device is at least 16.1% of the length of the working segment 380. According to some such embodiments, the length of the support segment 390 of the endovascular device is at least 16.2% of the length of the working segment 380. According to some such embodiments, the length of the support segment 390 of the endovascular device is at least 16.3% of the length of the working segment 380. According to some such embodiments, the length of the support segment 390 of the endovascular device is at least 16.4% of the length of the working segment 380. According to some such embodiments, the length of the support segment 390 of the endovascular device is at least 16.5% of the length of the working segment 380. According to some such embodiments, the length of the support segment 390 of the endovascular device is at least 16.6% of the length of the working segment 380. According to some such embodiments, the length of the support segment 390 of the endovascular device is at least 16.7% of the length of the working segment 380. According to some such embodiments, the length of the support segment 390 of the endovascular device is at least 16.8% of the length of the working segment 380. According to some such embodiments, the length of the support segment 390 of the endovascular device is at least 16.9% of the length of the working segment 380. According to some such embodiments, the length of the support segment 390 of the endovascular device is at least 17% of the length of the working segment 380.

According to some such embodiments, the length of the support segment 390 of the endovascular device is at least 17.1% of the length of the working segment 380. According to some such embodiments, the length of the support segment 390 of the endovascular device is at least 17.2% of the length of the working segment 380. According to some such embodiments, the length of the support segment 390 of the endovascular device is at least 17.3% of the length of the working segment 380. According to some such embodiments, the length of the support segment 390 of the endovascular device is at least 17.4% of the length of the working segment 380. According to some such embodiments, the length of the support segment 390 of the endovascular device is at least 17.5% of the length of the working segment 380. According to some such embodiments, the length of the support segment 390 of the endovascular device is at least 17.6% of the length of the working segment 380. According to some such embodiments, the length of the support segment 390 of the endovascular device is at least 17.7% of the length of the working segment 380. According to some such embodiments, the length of the support segment 390 of the endovascular device is at least 17.8% of the length of the working segment 380. According to some such embodiments, the length of the support segment 390 of the endovascular device is at least 17.9% of the length of the working segment 380. According to some such embodiments, the length of the support segment 390 of the endovascular device is at least 18% of the length of the working segment 380.

According to some such embodiments, the length of the support segment 390 of the endovascular device is at least 18.1% of the length of the working segment 380. According to some such embodiments, the length of the support segment 390 of the endovascular device is at least 18.2% of the length of the working segment 380. According to some such embodiments, the length of the support segment 390 of the endovascular device is at least 18.3% of the length of the working segment 380. According to some such embodiments, the length of the support segment 390 of the endovascular device is at least 18.4% of the length of the working segment 380. According to some such embodiments, the length of the support segment 390 of the endovascular device is at least 18.5% of the length of the working segment 380. According to some such embodiments, the length of the support segment 390 of the endovascular device is at least 18.6% of the length of the working segment 380. According to some such embodiments, the length of the support segment 390 of the endovascular device is at least 18.7% of the length of the working segment 380. According to some such embodiments, the length of the support segment 390 of the endovascular device is at least 18.8% of the length of the working segment 380. According to some such embodiments, the length of the support segment 390 of the endovascular device is at least 18.9% of the length of the working segment 380. According to some such embodiments, the length of the support segment 390 of the endovascular device is at least 19% of the length of the working segment 380.

According to some such embodiments, the length of the support segment 390 of the endovascular device is at least 19.1% of the length of the working segment 380. According to some such embodiments, the length of the support segment 390 of the endovascular device is at least 19.2% of the length of the working segment 380. According to some such embodiments, the length of the support segment 390 of the endovascular device is at least 19.3% of the length of the working segment 380. According to some such embodiments, the length of the support segment 390 of the endovascular device is at least 19.4% of the length of the working segment 380. According to some such embodiments, the length of the support segment 390 of the endovascular device is at least 19.5% of the length of the working segment 380. According to some such embodiments, the length of the support segment 390 of the endovascular device is at least 19.6% of the length of the working segment 380. According to some such embodiments, the length of the support segment 390 of the endovascular device is at least 19.7% of the length of the working segment 380. According to some such embodiments, the length of the support segment 390 of the endovascular device is at least 19.8% of the length of the working segment 380. According to some such embodiments, the length of the support segment 390 of the endovascular device is at least 19.9% of the length of the working segment 380. According to some such embodiments, the length of the support segment 390 of the endovascular device is at least 20% of the length of the working segment 380.

According to some such embodiments, the length of the support segment 390 of the endovascular device is at least 20.1% of the length of the working segment 380. According to some such embodiments, the length of the support segment 390 of the endovascular device is at least 20.2% of the length of the working segment 380. According to some such embodiments, the length of the support segment 390 of the endovascular device is at least 20.3% of the length of the working segment 380. According to some such embodiments, the length of the support segment 390 of the endovascular device is at least 20.4% of the length of the working segment 380. According to some such embodiments, the length of the support segment 390 of the endovascular device is at least 20.5% of the length of the working segment 380. According to some such embodiments, the length of the support segment 390 of the endovascular device is at least 20.6% of the length of the working segment 380. According to some such embodiments, the length of the support segment 390 of the endovascular device is at least 20.7% of the length of the working segment 380. According to some such embodiments, the length of the support segment 390 of the endovascular device is at least 20.8% of the length of the working segment 380. According to some such embodiments, the length of the support segment 390 of the endovascular device is at least 20.9% of the length of the working segment 380. According to some such embodiments, the length of the support segment 390 of the endovascular device is at least 21% of the length of the working segment 380.

According to some such embodiments, the length of the support segment 390 of the endovascular device is at least 21.1% of the length of the working segment 380. According to some such embodiments, the length of the support segment 390 of the endovascular device is at least 21.2% of the length of the working segment 380. According to some such embodiments, the length of the support segment 390 of the endovascular device is at least 21.3% of the length of the working segment 380. According to some such embodiments, the length of the support segment 390 of the endovascular device is at least 21.4% of the length of the working segment 380. According to some such embodiments, the length of the support segment 390 of the endovascular device is at least 21.5% of the length of the working segment 380. According to some such embodiments, the length of the support segment 390 of the endovascular device is at least 21.6% of the length of the working segment 380. According to some such embodiments, the length of the support segment 390 of the endovascular device is at least 21.7% of the length of the working segment 380. According to some such embodiments, the length of the support segment 390 of the endovascular device is at least 21.8% of the length of the working segment 380. According to some such embodiments, the length of the support segment 390 of the endovascular device is at least 21.9% of the length of the working segment 380. According to some such embodiments, the length of the support segment 390 of the endovascular device is at least 22% of the length of the working segment 380.

According to some such embodiments, the length of the support segment 390 of the endovascular device is at least 22.1% of the length of the working segment 380. According to some such embodiments, the length of the support segment 390 of the endovascular device is at least 22.2% of the length of the working segment 380. According to some such embodiments, the length of the support segment 390 of the endovascular device is at least 22.3% of the length of the working segment 380. According to some such embodiments, the length of the support segment 390 of the endovascular device is at least 22.4% of the length of the working segment 380. According to some such embodiments, the length of the support segment 390 of the endovascular device is at least 22.5% of the length of the working segment 380. According to some such embodiments, the length of the support segment 390 of the endovascular device is at least 22.6% of the length of the working segment 380. According to some such embodiments, the length of the support segment 390 of the endovascular device is at least 22.7% of the length of the working segment 380. According to some such embodiments, the length of the support segment 390 of the endovascular device is at least 22.8% of the length of the working segment 380. According to some such embodiments, the length of the support segment 390 of the endovascular device is at least 22.9% of the length of the working segment 380. According to some such embodiments, the length of the support segment 390 of the endovascular device is at least 23% of the length of the working segment 380.

According to some such embodiments, the length of the support segment 390 of the endovascular device is at least 23.1% of the length of the working segment 380. According to some such embodiments, the length of the support segment 390 of the endovascular device is at least 23.2% of the length of the working segment 380. According to some such embodiments, the length of the support segment 390 of the endovascular device is at least 23.3% of the length of the working segment 380. According to some such embodiments, the length of the support segment 390 of the endovascular device is at least 23.4% of the length of the working segment 380. According to some such embodiments, the length of the support segment 390 of the endovascular device is at least 23.5% of the length of the working segment 380. According to some such embodiments, the length of the support segment 390 of the endovascular device is at least 23.6% of the length of the working segment 380. According to some such embodiments, the length of the support segment 390 of the endovascular device is at least 23.7% of the length of the working segment 380. According to some such embodiments, the length of the support segment 390 of the endovascular device is at least 23.8% of the length of the working segment 380. According to some such embodiments, the length of the support segment 390 of the endovascular device is at least 23.9% of the length of the working segment 380. According to some such embodiments, the length of the support segment 390 of the endovascular device is at least 24% of the length of the working segment 380.

According to some such embodiments, the length of the support segment 390 of the endovascular device is at least 24.1% of the length of the working segment 380. According to some such embodiments, the length of the support segment 390 of the endovascular device is at least 24.2% of the length of the working segment 380. According to some such embodiments, the length of the support segment 390 of the endovascular device is at least 24.3% of the length of the working segment 380. According to some such embodiments, the length of the support segment 390 of the endovascular device is at least 24.4% of the length of the working segment 380. According to some such embodiments, the length of the support segment 390 of the endovascular device is at least 24.5% of the length of the working segment 380. According to some such embodiments, the length of the support segment 390 of the endovascular device is at least 24.6% of the length of the working segment 380. According to some such embodiments, the length of the support segment 390 of the endovascular device is at least 24.7% of the length of the working segment 380. According to some such embodiments, the length of the support segment 390 of the endovascular device is at least 24.8% of the length of the working segment 380. According to some such embodiments, the length of the support segment 390 of the endovascular device is at least 24.9% of the length of the working segment 380. According to some such embodiments, the length of the support segment 390 of the endovascular device is at least 25% of the length of the working segment 380.

According to some such embodiments, the length of the support segment 390 of the endovascular device is at least 25.1% of the length of the working segment 380. According to some such embodiments, the length of the support segment 390 of the endovascular device is at least 25.2% of the length of the working segment 380. According to some such embodiments, the length of the support segment 390 of the endovascular device is at least 25.3% of the length of the working segment 380. According to some such embodiments, the length of the support segment 390 of the endovascular device is at least 25.4% of the length of the working segment 380. According to some such embodiments, the length of the support segment 390 of the endovascular device is at least 25.5% of the length of the working segment 380. According to some such embodiments, the length of the support segment 390 of the endovascular device is at least 25.6% of the length of the working segment 380. According to some such embodiments, the length of the support segment 390 of the endovascular device is at least 25.7% of the length of the working segment 380. According to some such embodiments, the length of the support segment 390 of the endovascular device is at least 25.8% of the length of the working segment 380. According to some such embodiments, the length of the support segment 390 of the endovascular device is at least 25.9% of the length of the working segment 380. According to some such embodiments, the length of the support segment 390 of the endovascular device is at least 26% of the length of the working segment 380.

According to some such embodiments, the length of the support segment 390 of the endovascular device is at least 26.1% of the length of the working segment 380. According to some such embodiments, the length of the support segment 390 of the endovascular device is at least 26.2% of the length of the working segment 380. According to some such embodiments, the length of the support segment 390 of the endovascular device is at least 26.3% of the length of the working segment 380. According to some such embodiments, the length of the support segment 390 of the endovascular device is at least 26.4% of the length of the working segment 380. According to some such embodiments, the length of the support segment 390 of the endovascular device is at least 26.5% of the length of the working segment 380. According to some such embodiments, the length of the support segment 390 of the endovascular device is at least 26.6% of the length of the working segment 380. According to some such embodiments, the length of the support segment 390 of the endovascular device is at least 26.7% of the length of the working segment 380. According to some such embodiments, the length of the support segment 390 of the endovascular device is at least 26.8% of the length of the working segment 380. According to some such embodiments, the length of the support segment 390 of the endovascular device is at least 26.9% of the length of the working segment 380. According to some such embodiments, the length of the support segment 390 of the endovascular device is at least 27% of the length of the working segment 380.

According to some such embodiments, the length of the support segment 390 of the endovascular device is at least 27.1% of the length of the working segment 380. According to some such embodiments, the length of the support segment 390 of the endovascular device is at least 27.2% of the length of the working segment 380. According to some such embodiments, the length of the support segment 390 of the endovascular device is at least 27.3% of the length of the working segment 380. According to some such embodiments, the length of the support segment 390 of the endovascular device is at least 27.4% of the length of the working segment 380. According to some such embodiments, the length of the support segment 390 of the endovascular device is at least 27.5% of the length of the working segment 380. According to some such embodiments, the length of the support segment 390 of the endovascular device is at least 27.6% of the length of the working segment 380. According to some such embodiments, the length of the support segment 390 of the endovascular device is at least 27.7% of the length of the working segment 380. According to some such embodiments, the length of the support segment 390 of the endovascular device is at least 27.8% of the length of the working segment 380. According to some such embodiments, the length of the support segment 390 of the endovascular device is at least 27.9% of the length of the working segment 380. According to some such embodiments, the length of the support segment 390 of the endovascular device is at least 28% of the length of the working segment 380.

According to some such embodiments, the length of the support segment 390 of the endovascular device is at least 28.1% of the length of the working segment 380. According to some such embodiments, the length of the support segment 390 of the endovascular device is at least 28.2% of the length of the working segment 380. According to some such embodiments, the length of the support segment 390 of the endovascular device is at least 28.3% of the length of the working segment 380. According to some such embodiments, the length of the support segment 390 of the endovascular device is at least 28.4% of the length of the working segment 380. According to some such embodiments, the length of the support segment 390 of the endovascular device is at least 28.5% of the length of the working segment 380. According to some such embodiments, the length of the support segment 390 of the endovascular device is at least 28.6% of the length of the working segment 380. According to some such embodiments, the length of the support segment 390 of the endovascular device is at least 28.7% of the length of the working segment 380. According to some such embodiments, the length of the support segment 390 of the endovascular device is at least 28.8% of the length of the working segment 380. According to some such embodiments, the length of the support segment 390 of the endovascular device is at least 28.9% of the length of the working segment 380. According to some such embodiments, the length of the support segment 390 of the endovascular device is at least 29% of the length of the working segment 380.

According to some such embodiments, the length of the support segment 390 of the endovascular device is at least 29.1% of the length of the working segment 380. According to some such embodiments, the length of the support segment 390 of the endovascular device is at least 29.2% of the length of the working segment 380. According to some such embodiments, the length of the support segment 390 of the endovascular device is at least 29.3% of the length of the working segment 380. According to some such embodiments, the length of the support segment 390 of the endovascular device is at least 29.4% of the length of the working segment 380. According to some such embodiments, the length of the support segment 390 of the endovascular device is at least 29.5% of the length of the working segment 380. According to some such embodiments, the length of the support segment 390 of the endovascular device is at least 29.6% of the length of the working segment 380. According to some such embodiments, the length of the support segment 390 of the endovascular device is at least 29.7% of the length of the working segment 380. According to some such embodiments, the length of the support segment 390 of the endovascular device is at least 29.8% of the length of the working segment 380. According to some such embodiments, the length of the support segment 390 of the endovascular device is at least 29.9% of the length of the working segment 380. According to some such embodiments, the length of the support segment 390 of the endovascular device is at least 30% of the length of the working segment 380.

According to some such embodiments, the length of the support segment 390 of the endovascular device is at least 30.1% of the length of the working segment 380. According to some such embodiments, the length of the support segment 390 of the endovascular device is at least 30.2% of the length of the working segment 380. According to some such embodiments, the length of the support segment 390 of the endovascular device is at least 30.3% of the length of the working segment 380. According to some such embodiments, the length of the support segment 390 of the endovascular device is at least 30.4% of the length of the working segment 380. According to some such embodiments, the length of the support segment 390 of the endovascular device is at least 30.5% of the length of the working segment 380. According to some such embodiments, the length of the support segment 390 of the endovascular device is at least 30.6% of the length of the working segment 380. According to some such embodiments, the length of the support segment 390 of the endovascular device is at least 30.7% of the length of the working segment 380. According to some such embodiments, the length of the support segment 390 of the endovascular device is at least 30.8% of the length of the working segment 380. According to some such embodiments, the length of the support segment 390 of the endovascular device is at least 30.9% of the length of the working segment 380. According to some such embodiments, the length of the support segment 390 of the endovascular device is at least 31% of the length of the working segment 380.

According to some such embodiments, the length of the support segment 390 of the endovascular device is at least 31.1% of the length of the working segment 380. According to some such embodiments, the length of the support segment 390 of the endovascular device is at least 31.2% of the length of the working segment 380. According to some such embodiments, the length of the support segment 390 of the endovascular device is at least 31.3% of the length of the working segment 380. According to some such embodiments, the length of the support segment 390 of the endovascular device is at least 31.4% of the length of the working segment 380. According to some such embodiments, the length of the support segment 390 of the endovascular device is at least 31.5% of the length of the working segment 380. According to some such embodiments, the length of the support segment 390 of the endovascular device is at least 31.6% of the length of the working segment 380. According to some such embodiments, the length of the support segment 390 of the endovascular device is at least 31.7% of the length of the working segment 380. According to some such embodiments, the length of the support segment 390 of the endovascular device is at least 31.8% of the length of the working segment 380. According to some such embodiments, the length of the support segment 390 of the endovascular device is at least 31.9% of the length of the working segment 380. According to some such embodiments, the length of the support segment 390 of the endovascular device is at least 32% of the length of the working segment 380.

According to some such embodiments, the length of the support segment 390 of the endovascular device is at least 32.1% of the length of the working segment 380. According to some such embodiments, the length of the support segment 390 of the endovascular device is at least 32.2% of the length of the working segment 380. According to some such embodiments, the length of the support segment 390 of the endovascular device is at least 32.3% of the length of the working segment 380. According to some such embodiments, the length of the support segment 390 of the endovascular device is at least 32.4% of the length of the working segment 380. According to some such embodiments, the length of the support segment 390 of the endovascular device is at least 32.5% of the length of the working segment 380. According to some such embodiments, the length of the support segment 390 of the endovascular device is at least 32.6% of the length of the working segment 380. According to some such embodiments, the length of the support segment 390 of the endovascular device is at least 32.7% of the length of the working segment 380. According to some such embodiments, the length of the support segment 390 of the endovascular device is at least 32.8% of the length of the working segment 380. According to some such embodiments, the length of the support segment 390 of the endovascular device is at least 32.9% of the length of the working segment 380. According to some such embodiments, the length of the support segment 390 of the endovascular device is at least 33% of the length of the working segment 380.

According to some such embodiments, the length of the support segment 390 of the endovascular device is at least 33.1% of the length of the working segment 380. According to some such embodiments, the length of the support segment 390 of the endovascular device is at least 33.2% of the length of the working segment 380. According to some such embodiments, the length of the support segment 390 of the endovascular device is at least 33.3% of the length of the working segment 380. According to some such embodiments, the length of the support segment 390 of the endovascular device is at least 33.4% of the length of the working segment 380. According to some such embodiments, the length of the support segment 390 of the endovascular device is at least 33.5% of the length of the working segment 380. According to some such embodiments, the length of the support segment 390 of the endovascular device is at least 33.6% of the length of the working segment 380. According to some such embodiments, the length of the support segment 390 of the endovascular device is at least 33.7% of the length of the working segment 380. According to some such embodiments, the length of the support segment 390 of the endovascular device is at least 33.8% of the length of the working segment 380. According to some such embodiments, the length of the support segment 390 of the endovascular device is at least 33.9% of the length of the working segment 380. According to some such embodiments, the length of the support segment 390 of the endovascular device is at least 34% of the length of the working segment 380.

According to some such embodiments, the length of the support segment 390 of the endovascular device is at least 34.1% of the length of the working segment 380. According to some such embodiments, the length of the support segment 390 of the endovascular device is at least 34.2% of the length of the working segment 380. According to some such embodiments, the length of the support segment 390 of the endovascular device is at least 34.3% of the length of the working segment 380. According to some such embodiments, the length of the support segment 390 of the endovascular device is at least 34.4% of the length of the working segment 380. According to some such embodiments, the length of the support segment 390 of the endovascular device is at least 34.5% of the length of the working segment 380. According to some such embodiments, the length of the support segment 390 of the endovascular device is at least 34.6% of the length of the working segment 380. According to some such embodiments, the length of the support segment 390 of the endovascular device is at least 34.7% of the length of the working segment 380. According to some such embodiments, the length of the support segment 390 of the endovascular device is at least 34.8% of the length of the working segment 380. According to some such embodiments, the length of the support segment 390 of the endovascular device is at least 34.9% of the length of the working segment 380. According to some such embodiments, the length of the support segment 390 of the endovascular device is at least 35% of the length of the working segment 380.

According to some such embodiments, the length of the support segment 390 of the endovascular device is at least 35.1% of the length of the working segment 380. According to some such embodiments, the length of the support segment 390 of the endovascular device is at least 35.2% of the length of the working segment 380. According to some such embodiments, the length of the support segment 390 of the endovascular device is at least 35.3% of the length of the working segment 380. According to some such embodiments, the length of the support segment 390 of the endovascular device is at least 35.4% of the length of the working segment 380. According to some such embodiments, the length of the support segment 390 of the endovascular device is at least 35.5% of the length of the working segment 380. According to some such embodiments, the length of the support segment 390 of the endovascular device is at least 35.6% of the length of the working segment 380. According to some such embodiments, the length of the support segment 390 of the endovascular device is at least 35.7% of the length of the working segment 380. According to some such embodiments, the length of the support segment 390 of the endovascular device is at least 35.8% of the length of the working segment 380. According to some such embodiments, the length of the support segment 390 of the endovascular device is at least 35.9% of the length of the working segment 380. According to some such embodiments, the length of the support segment 390 of the endovascular device is at least 36% of the length of the working segment 380.

According to some such embodiments, the length of the support segment 390 of the endovascular device is at least 36.1% of the length of the working segment 380. According to some such embodiments, the length of the support segment 390 of the endovascular device is at least 36.2% of the length of the working segment 380. According to some such embodiments, the length of the support segment 390 of the endovascular device is at least 36.3% of the length of the working segment 380. According to some such embodiments, the length of the support segment 390 of the endovascular device is at least 36.4% of the length of the working segment 380. According to some such embodiments, the length of the support segment 390 of the endovascular device is at least 36.5% of the length of the working segment 380. According to some such embodiments, the length of the support segment 390 of the endovascular device is at least 36.6% of the length of the working segment 380. According to some such embodiments, the length of the support segment 390 of the endovascular device is at least 36.7% of the length of the working segment 380. According to some such embodiments, the length of the support segment 390 of the endovascular device is at least 36.8% of the length of the working segment 380. According to some such embodiments, the length of the support segment 390 of the endovascular device is at least 36.9% of the length of the working segment 380. According to some such embodiments, the length of the support segment 390 of the endovascular device is at least 37% of the length of the working segment 380.

According to some such embodiments, the length of the support segment 390 of the endovascular device is at least 37.1% of the length of the working segment 380. According to some such embodiments, the length of the support segment 390 of the endovascular device is at least 37.2% of the length of the working segment 380. According to some such embodiments, the length of the support segment 390 of the endovascular device is at least 37.3% of the length of the working segment 380. According to some such embodiments, the length of the support segment 390 of the endovascular device is at least 37.4% of the length of the working segment 380. According to some such embodiments, the length of the support segment 390 of the endovascular device is at least 37.5% of the length of the working segment 380. According to some such embodiments, the length of the support segment 390 of the endovascular device is at least 37.6% of the length of the working segment 380. According to some such embodiments, the length of the support segment 390 of the endovascular device is at least 37.7% of the length of the working segment 380. According to some such embodiments, the length of the support segment 390 of the endovascular device is at least 37.8% of the length of the working segment 380. According to some such embodiments, the length of the support segment 390 of the endovascular device is at least 37.9% of the length of the working segment 380. According to some such embodiments, the length of the support segment 390 of the endovascular device is at least 38% of the length of the working segment 380.

According to some such embodiments, the length of the support segment 390 of the endovascular device is at least 38.1% of the length of the working segment 380. According to some such embodiments, the length of the support segment 390 of the endovascular device is at least 38.2% of the length of the working segment 380. According to some such embodiments, the length of the support segment 390 of the endovascular device is at least 38.3% of the length of the working segment 380. According to some such embodiments, the length of the support segment 390 of the endovascular device is at least 38.4% of the length of the working segment 380. According to some such embodiments, the length of the support segment 390 of the endovascular device is at least 38.5% of the length of the working segment 380. According to some such embodiments, the length of the support segment 390 of the endovascular device is at least 38.6% of the length of the working segment 380. According to some such embodiments, the length of the support segment 390 of the endovascular device is at least 38.7% of the length of the working segment 380. According to some such embodiments, the length of the support segment 390 of the endovascular device is at least 38.8% of the length of the working segment 380. According to some such embodiments, the length of the support segment 390 of the endovascular device is at least 38.9% of the length of the working segment 380. According to some such embodiments, the length of the support segment 390 of the endovascular device is at least 39% of the length of the working segment 380.

According to some such embodiments, the length of the support segment 390 of the endovascular device is at least 39.1% of the length of the working segment 380. According to some such embodiments, the length of the support segment 390 of the endovascular device is at least 39.2% of the length of the working segment 380. According to some such embodiments, the length of the support segment 390 of the endovascular device is at least 39.3% of the length of the working segment 380. According to some such embodiments, the length of the support segment 390 of the endovascular device is at least 39.4% of the length of the working segment 380. According to some such embodiments, the length of the support segment 390 of the endovascular device is at least 39.5% of the length of the working segment 380. According to some such embodiments, the length of the support segment 390 of the endovascular device is at least 39.6% of the length of the working segment 380. According to some such embodiments, the length of the support segment 390 of the endovascular device is at least 39.7% of the length of the working segment 380. According to some such embodiments, the length of the support segment 390 of the endovascular device is at least 39.8% of the length of the working segment 380. According to some such embodiments, the length of the support segment 390 of the endovascular device is at least 39.9% of the length of the working segment 380. According to some such embodiments, the length of the support segment 390 of the endovascular device is at least 40% of the length of the working segment 380.

According to some such embodiments, the length of the support segment 390 of the endovascular device is at least 40.1% of the length of the working segment 380. According to some such embodiments, the length of the support segment 390 of the endovascular device is at least 40.2% of the length of the working segment 380. According to some such embodiments, the length of the support segment 390 of the endovascular device is at least 40.3% of the length of the working segment 380. According to some such embodiments, the length of the support segment 390 of the endovascular device is at least 40.4% of the length of the working segment 380. According to some such embodiments, the length of the support segment 390 of the endovascular device is at least 40.5% of the length of the working segment 380. According to some such embodiments, the length of the support segment 390 of the endovascular device is at least 40.6% of the length of the working segment 380. According to some such embodiments, the length of the support segment 390 of the endovascular device is at least 40.7% of the length of the working segment 380. According to some such embodiments, the length of the support segment 390 of the endovascular device is at least 40.8% of the length of the working segment 380. According to some such embodiments, the length of the support segment 390 of the endovascular device is at least 40.9% of the length of the working segment 380. According to some such embodiments, the length of the support segment 390 of the endovascular device is at least 41% of the length of the working segment 380.

According to some such embodiments, the length of the support segment 390 of the endovascular device is at least 41.1% of the length of the working segment 380. According to some such embodiments, the length of the support segment 390 of the endovascular device is at least 41.2% of the length of the working segment 380. According to some such embodiments, the length of the support segment 390 of the endovascular device is at least 41.3% of the length of the working segment 380. According to some such embodiments, the length of the support segment 390 of the endovascular device is at least 41.4% of the length of the working segment 380. According to some such embodiments, the length of the support segment 390 of the endovascular device is at least 41.5% of the length of the working segment 380. According to some such embodiments, the length of the support segment 390 of the endovascular device is at least 41.6% of the length of the working segment 380. According to some such embodiments, the length of the support segment 390 of the endovascular device is at least 41.7% of the length of the working segment 380. According to some such embodiments, the length of the support segment 390 of the endovascular device is at least 41.8% of the length of the working segment 380. According to some such embodiments, the length of the support segment 390 of the endovascular device is at least 41.9% of the length of the working segment 380. According to some such embodiments, the length of the support segment 390 of the endovascular device is at least 42% of the length of the working segment 380.

According to some such embodiments, the length of the support segment 390 of the endovascular device is at least 42.1% of the length of the working segment 380. According to some such embodiments, the length of the support segment 390 of the endovascular device is at least 42.2% of the length of the working segment 380. According to some such embodiments, the length of the support segment 390 of the endovascular device is at least 42.3% of the length of the working segment 380. According to some such embodiments, the length of the support segment 390 of the endovascular device is at least 42.4% of the length of the working segment 380. According to some such embodiments, the length of the support segment 390 of the endovascular device is at least 42.5% of the length of the working segment 380. According to some such embodiments, the length of the support segment 390 of the endovascular device is at least 42.6% of the length of the working segment 380. According to some such embodiments, the length of the support segment 390 of the endovascular device is at least 42.7% of the length of the working segment 380. According to some such embodiments, the length of the support segment 390 of the endovascular device is at least 42.8% of the length of the working segment 380. According to some such embodiments, the length of the support segment 390 of the endovascular device is at least 42.9% of the length of the working segment 380. According to some such embodiments, the length of the support segment 390 of the endovascular device is at least 43% of the length of the working segment 380.

According to some such embodiments, the length of the support segment 390 of the endovascular device is at least 43.1% of the length of the working segment 380. According to some such embodiments, the length of the support segment 390 of the endovascular device is at least 43.2% of the length of the working segment 380. According to some such embodiments, the length of the support segment 390 of the endovascular device is at least 43.3% of the length of the working segment 380. According to some such embodiments, the length of the support segment 390 of the endovascular device is at least 43.4% of the length of the working segment 380. According to some such embodiments, the length of the support segment 390 of the endovascular device is at least 43.5% of the length of the working segment 380. According to some such embodiments, the length of the support segment 390 of the endovascular device is at least 43.6% of the length of the working segment 380. According to some such embodiments, the length of the support segment 390 of the endovascular device is at least 43.7% of the length of the working segment 380. According to some such embodiments, the length of the support segment 390 of the endovascular device is at least 43.8% of the length of the working segment 380. According to some such embodiments, the length of the support segment 390 of the endovascular device is at least 43.9% of the length of the working segment 380. According to some such embodiments, the length of the support segment 390 of the endovascular device is at least 44% of the length of the working segment 380.

According to some such embodiments, the length of the support segment 390 of the endovascular device is at least 44.1% of the length of the working segment 380. According to some such embodiments, the length of the support segment 390 of the endovascular device is at least 44.2% of the length of the working segment 380. According to some such embodiments, the length of the support segment 390 of the endovascular device is at least 44.3% of the length of the working segment 380. According to some such embodiments, the length of the support segment 390 of the endovascular device is at least 44.4% of the length of the working segment 380. According to some such embodiments, the length of the support segment 390 of the endovascular device is at least 44.5% of the length of the working segment 380. According to some such embodiments, the length of the support segment 390 of the endovascular device is at least 44.6% of the length of the working segment 380. According to some such embodiments, the length of the support segment 390 of the endovascular device is at least 44.7% of the length of the working segment 380. According to some such embodiments, the length of the support segment 390 of the endovascular device is at least 44.8% of the length of the working segment 380. According to some such embodiments, the length of the support segment 390 of the endovascular device is at least 44.9% of the length of the working segment 380. According to some such embodiments, the length of the support segment 390 of the endovascular device is at least 45% of the length of the working segment 380.

According to some such embodiments, the length of the support segment 390 of the endovascular device is at least 45.1% of the length of the working segment 380. According to some such embodiments, the length of the support segment 390 of the endovascular device is at least 45.2% of the length of the working segment 380. According to some such embodiments, the length of the support segment 390 of the endovascular device is at least 45.3% of the length of the working segment 380. According to some such embodiments, the length of the support segment 390 of the endovascular device is at least 45.4% of the length of the working segment 380. According to some such embodiments, the length of the support segment 390 of the endovascular device is at least 45.5% of the length of the working segment 380. According to some such embodiments, the length of the support segment 390 of the endovascular device is at least 45.6% of the length of the working segment 380. According to some such embodiments, the length of the support segment 390 of the endovascular device is at least 45.7% of the length of the working segment 380. According to some such embodiments, the length of the support segment 390 of the endovascular device is at least 45.8% of the length of the working segment 380. According to some such embodiments, the length of the support segment 390 of the endovascular device is at least 45.9% of the length of the working segment 380. According to some such embodiments, the length of the support segment 390 of the endovascular device is at least 46% of the length of the working segment 380.

According to some such embodiments, the length of the support segment 390 of the endovascular device is at least 46.1% of the length of the working segment 380. According to some such embodiments, the length of the support segment 390 of the endovascular device is at least 46.2% of the length of the working segment 380. According to some such embodiments, the length of the support segment 390 of the endovascular device is at least 46.3% of the length of the working segment 380. According to some such embodiments, the length of the support segment 390 of the endovascular device is at least 46.4% of the length of the working segment 380. According to some such embodiments, the length of the support segment 390 of the endovascular device is at least 46.5% of the length of the working segment 380. According to some such embodiments, the length of the support segment 390 of the endovascular device is at least 46.6% of the length of the working segment 380. According to some such embodiments, the length of the support segment 390 of the endovascular device is at least 46.7% of the length of the working segment 380. According to some such embodiments, the length of the support segment 390 of the endovascular device is at least 46.8% of the length of the working segment 380. According to some such embodiments, the length of the support segment 390 of the endovascular device is at least 46.9% of the length of the working segment 380. According to some such embodiments, the length of the support segment 390 of the endovascular device is at least 47% of the length of the working segment 380.

According to some such embodiments, the length of the support segment 390 of the endovascular device is at least 47.1% of the length of the working segment 380. According to some such embodiments, the length of the support segment 390 of the endovascular device is at least 47.2% of the length of the working segment 380. According to some such embodiments, the length of the support segment 390 of the endovascular device is at least 47.3% of the length of the working segment 380. According to some such embodiments, the length of the support segment 390 of the endovascular device is at least 47.4% of the length of the working segment 380. According to some such embodiments, the length of the support segment 390 of the endovascular device is at least 47.5% of the length of the working segment 380. According to some such embodiments, the length of the support segment 390 of the endovascular device is at least 47.6% of the length of the working segment 380. According to some such embodiments, the length of the support segment 390 of the endovascular device is at least 47.7% of the length of the working segment 380. According to some such embodiments, the length of the support segment 390 of the endovascular device is at least 47.8% of the length of the working segment 380. According to some such embodiments, the length of the support segment 390 of the endovascular device is at least 47.9% of the length of the working segment 380. According to some such embodiments, the length of the support segment 390 of the endovascular device is at least 48% of the length of the working segment 380.

According to some such embodiments, the length of the support segment 390 of the endovascular device is at least 48.1% of the length of the working segment 380. According to some such embodiments, the length of the support segment 390 of the endovascular device is at least 48.2% of the length of the working segment 380. According to some such embodiments, the length of the support segment 390 of the endovascular device is at least 48.3% of the length of the working segment 380. According to some such embodiments, the length of the support segment 390 of the endovascular device is at least 48.4% of the length of the working segment 380. According to some such embodiments, the length of the support segment 390 of the endovascular device is at least 48.5% of the length of the working segment 380. According to some such embodiments, the length of the support segment 390 of the endovascular device is at least 48.6% of the length of the working segment 380. According to some such embodiments, the length of the support segment 390 of the endovascular device is at least 48.7% of the length of the working segment 380. According to some such embodiments, the length of the support segment 390 of the endovascular device is at least 48.8% of the length of the working segment 380. According to some such embodiments, the length of the support segment 390 of the endovascular device is at least 48.9% of the length of the working segment 380. According to some such embodiments, the length of the support segment 390 of the endovascular device is at least 49% of the length of the working segment 380.

According to some such embodiments, the length of the support segment 390 of the endovascular device is at least 49.1% of the length of the working segment 380. According to some such embodiments, the length of the support segment 390 of the endovascular device is at least 49.2% of the length of the working segment 380. According to some such embodiments, the length of the support segment 390 of the endovascular device is at least 49.3% of the length of the working segment 380. According to some such embodiments, the length of the support segment 390 of the endovascular device is at least 49.4% of the length of the working segment 380. According to some such embodiments, the length of the support segment 390 of the endovascular device is at least 49.5% of the length of the working segment 380. According to some such embodiments, the length of the support segment 390 of the endovascular device is at least 49.6% of the length of the working segment 380. According to some such embodiments, the length of the support segment 390 of the endovascular device is at least 49.7% of the length of the working segment 380. According to some such embodiments, the length of the support segment 390 of the endovascular device is at least 49.8% of the length of the working segment 380. According to some such embodiments, the length of the support segment 390 of the endovascular device is at least 49.9% of the length of the working segment 380. According to some such embodiments, the length of the support segment 390 of the endovascular device is at least 50% of the length of the working segment 380.

According to some such embodiments, the length of the support segment 390 of the endovascular device is at least 50.1% of the length of the working segment 380. According to some such embodiments, the length of the support segment 390 of the endovascular device is at least 50.2% of the length of the working segment 380. According to some such embodiments, the length of the support segment 390 of the endovascular device is at least 50.3% of the length of the working segment 380. According to some such embodiments, the length of the support segment 390 of the endovascular device is at least 50.4% of the length of the working segment 380. According to some such embodiments, the length of the support segment 390 of the endovascular device is at least 50.5% of the length of the working segment 380. According to some such embodiments, the length of the support segment 390 of the endovascular device is at least 50.6% of the length of the working segment 380. According to some such embodiments, the length of the support segment 390 of the endovascular device is at least 50.7% of the length of the working segment 380. According to some such embodiments, the length of the support segment 390 of the endovascular device is at least 50.8% of the length of the working segment 380. According to some such embodiments, the length of the support segment 390 of the endovascular device is at least 50.9% of the length of the working segment 380. According to some such embodiments, the length of the support segment 390 of the endovascular device is at least 51% of the length of the working segment 380.

According to some such embodiments, the length of the support segment 390 of the endovascular device is at least 51.1% of the length of the working segment 380. According to some such embodiments, the length of the support segment 390 of the endovascular device is at least 51.2% of the length of the working segment 380. According to some such embodiments, the length of the support segment 390 of the endovascular device is at least 51.3% of the length of the working segment 380. According to some such embodiments, the length of the support segment 390 of the endovascular device is at least 51.4% of the length of the working segment 380. According to some such embodiments, the length of the support segment 390 of the endovascular device is at least 51.5% of the length of the working segment 380. According to some such embodiments, the length of the support segment 390 of the endovascular device is at least 51.6% of the length of the working segment 380. According to some such embodiments, the length of the support segment 390 of the endovascular device is at least 51.7% of the length of the working segment 380. According to some such embodiments, the length of the support segment 390 of the endovascular device is at least 51.8% of the length of the working segment 380. According to some such embodiments, the length of the support segment 390 of the endovascular device is at least 51.9% of the length of the working segment 380. According to some such embodiments, the length of the support segment 390 of the endovascular device is at least 52% of the length of the working segment 380.

According to some such embodiments, the length of the support segment 390 of the endovascular device is at least 52.1% of the length of the working segment 380. According to some such embodiments, the length of the support segment 390 of the endovascular device is at least 52.2% of the length of the working segment 380. According to some such embodiments, the length of the support segment 390 of the endovascular device is at least 52.3% of the length of the working segment 380. According to some such embodiments, the length of the support segment 390 of the endovascular device is at least 52.4% of the length of the working segment 380. According to some such embodiments, the length of the support segment 390 of the endovascular device is at least 52.5% of the length of the working segment 380. According to some such embodiments, the length of the support segment 390 of the endovascular device is at least 52.6% of the length of the working segment 380. According to some such embodiments, the length of the support segment 390 of the endovascular device is at least 52.7% of the length of the working segment 380. According to some such embodiments, the length of the support segment 390 of the endovascular device is at least 52.8% of the length of the working segment 380. According to some such embodiments, the length of the support segment 390 of the endovascular device is at least 52.9% of the length of the working segment 380. According to some such embodiments, the length of the support segment 390 of the endovascular device is at least 53% of the length of the working segment 380.

According to some such embodiments, the length of the support segment 390 of the endovascular device is at least 53.1% of the length of the working segment 380. According to some such embodiments, the length of the support segment 390 of the endovascular device is at least 53.2% of the length of the working segment 380. According to some such embodiments, the length of the support segment 390 of the endovascular device is at least 53.3% of the length of the working segment 380. According to some such embodiments, the length of the support segment 390 of the endovascular device is at least 53.4% of the length of the working segment 380. According to some such embodiments, the length of the support segment 390 of the endovascular device is at least 53.5% of the length of the working segment 380. According to some such embodiments, the length of the support segment 390 of the endovascular device is at least 53.6% of the length of the working segment 380. According to some such embodiments, the length of the support segment 390 of the endovascular device is at least 53.7% of the length of the working segment 380. According to some such embodiments, the length of the support segment 390 of the endovascular device is at least 53.8% of the length of the working segment 380. According to some such embodiments, the length of the support segment 390 of the endovascular device is at least 53.9% of the length of the working segment 380. According to some such embodiments, the length of the support segment 390 of the endovascular device is at least 54% of the length of the working segment 380.

According to some such embodiments, the length of the support segment 390 of the endovascular device is at least 54.1% of the length of the working segment 380. According to some such embodiments, the length of the support segment 390 of the endovascular device is at least 54.2% of the length of the working segment 380. According to some such embodiments, the length of the support segment 390 of the endovascular device is at least 54.3% of the length of the working segment 380. According to some such embodiments, the length of the support segment 390 of the endovascular device is at least 54.4% of the length of the working segment 380. According to some such embodiments, the length of the support segment 390 of the endovascular device is at least 54.5% of the length of the working segment 380. According to some such embodiments, the length of the support segment 390 of the endovascular device is at least 54.6% of the length of the working segment 380. According to some such embodiments, the length of the support segment 390 of the endovascular device is at least 54.7% of the length of the working segment 380. According to some such embodiments, the length of the support segment 390 of the endovascular device is at least 54.8% of the length of the working segment 380. According to some such embodiments, the length of the support segment 390 of the endovascular device is at least 54.9% of the length of the working segment 380. According to some such embodiments, the length of the support segment 390 of the endovascular device is at least 55% of the length of the working segment 380.

According to some such embodiments, the length of the support segment 390 of the endovascular device is at least 55.1% of the length of the working segment 380. According to some such embodiments, the length of the support segment 390 of the endovascular device is at least 55.2% of the length of the working segment 380. According to some such embodiments, the length of the support segment 390 of the endovascular device is at least 55.3% of the length of the working segment 380. According to some such embodiments, the length of the support segment 390 of the endovascular device is at least 55.4% of the length of the working segment 380. According to some such embodiments, the length of the support segment 390 of the endovascular device is at least 55.5% of the length of the working segment 380. According to some such embodiments, the length of the support segment 390 of the endovascular device is at least 55.6% of the length of the working segment 380. According to some such embodiments, the length of the support segment 390 of the endovascular device is at least 55.7% of the length of the working segment 380. According to some such embodiments, the length of the support segment 390 of the endovascular device is at least 55.8% of the length of the working segment 380. According to some such embodiments, the length of the support segment 390 of the endovascular device is at least 55.9% of the length of the working segment 380. According to some such embodiments, the length of the support segment 390 of the endovascular device is at least 56% of the length of the working segment 380.

According to some such embodiments, the length of the support segment 390 of the endovascular device is at least 56.1% of the length of the working segment 380. According to some such embodiments, the length of the support segment 390 of the endovascular device is at least 56.2% of the length of the working segment 380. According to some such embodiments, the length of the support segment 390 of the endovascular device is at least 56.3% of the length of the working segment 380. According to some such embodiments, the length of the support segment 390 of the endovascular device is at least 56.4% of the length of the working segment 380. According to some such embodiments, the length of the support segment 390 of the endovascular device is at least 6.5% of the length of the working segment 380. According to some such embodiments, the length of the support segment 390 of the endovascular device is at least 56.6% of the length of the working segment 380. According to some such embodiments, the length of the support segment 390 of the endovascular device is at least 56.7% of the length of the working segment 380. According to some such embodiments, the length of the support segment 390 of the endovascular device is at least 56.8% of the length of the working segment 380. According to some such embodiments, the length of the support segment 390 of the endovascular device is at least 56.9% of the length of the working segment 380. According to some such embodiments, the length of the support segment 390 of the endovascular device is at least 57% of the length of the working segment 380.

According to some such embodiments, the length of the support segment 390 of the endovascular device is at least 57.1% of the length of the working segment 380. According to some such embodiments, the length of the support segment 390 of the endovascular device is at least 57.2% of the length of the working segment 380. According to some such embodiments, the length of the support segment 390 of the endovascular device is at least 57.3% of the length of the working segment 380. According to some such embodiments, the length of the support segment 390 of the endovascular device is at least 57.4% of the length of the working segment 380. According to some such embodiments, the length of the support segment 390 of the endovascular device is at least 57.5% of the length of the working segment 380. According to some such embodiments, the length of the support segment 390 of the endovascular device is at least 57.6% of the length of the working segment 380. According to some such embodiments, the length of the support segment 390 of the endovascular device is at least 57.7% of the length of the working segment 380. According to some such embodiments, the length of the support segment 390 of the endovascular device is at least 57.8% of the length of the working segment 380. According to some such embodiments, the length of the support segment 390 of the endovascular device is at least 57.9% of the length of the working segment 380. According to some such embodiments, the length of the support segment 390 of the endovascular device is at least 58% of the length of the working segment 380.

According to some such embodiments, the length of the support segment 390 of the endovascular device is at least 58.1% of the length of the working segment 380. According to some such embodiments, the length of the support segment 390 of the endovascular device is at least 58.2% of the length of the working segment 380. According to some such embodiments, the length of the support segment 390 of the endovascular device is at least 58.3% of the length of the working segment 380. According to some such embodiments, the length of the support segment 390 of the endovascular device is at least 58.4% of the length of the working segment 380. According to some such embodiments, the length of the support segment 390 of the endovascular device is at least 58.5% of the length of the working segment 380. According to some such embodiments, the length of the support segment 390 of the endovascular device is at least 58.6% of the length of the working segment 380. According to some such embodiments, the length of the support segment 390 of the endovascular device is at least 58.7% of the length of the working segment 380. According to some such embodiments, the length of the support segment 390 of the endovascular device is at least 58.8% of the length of the working segment 380. According to some such embodiments, the length of the support segment 390 of the endovascular device is at least 58.9% of the length of the working segment 380. According to some such embodiments, the length of the support segment 390 of the endovascular device is at least 59% of the length of the working segment 380.

According to some such embodiments, the length of the support segment 390 of the endovascular device is at least 59.1% of the length of the working segment 380. According to some such embodiments, the length of the support segment 390 of the endovascular device is at least 59.2% of the length of the working segment 380. According to some such embodiments, the length of the support segment 390 of the endovascular device is at least 59.3% of the length of the working segment 380. According to some such embodiments, the length of the support segment 390 of the endovascular device is at least 59.4% of the length of the working segment 380. According to some such embodiments, the length of the support segment 390 of the endovascular device is at least 59.5% of the length of the working segment 380. According to some such embodiments, the length of the support segment 390 of the endovascular device is at least 59.6% of the length of the working segment 380. According to some such embodiments, the length of the support segment 390 of the endovascular device is at least 59.7% of the length of the working segment 380. According to some such embodiments, the length of the support segment 390 of the endovascular device is at least 59.8% of the length of the working segment 380. According to some such embodiments, the length of the support segment 390 of the endovascular device is at least 59.9% of the length of the working segment 380. According to some such embodiments, the length of the support segment 390 of the endovascular device is at least 60% of the length of the working segment 380.

According to some such embodiments, the length of the support segment 390 of the endovascular device is at least 60.1% of the length of the working segment 380. According to some such embodiments, the length of the support segment 390 of the endovascular device is at least 60.2% of the length of the working segment 380. According to some such embodiments, the length of the support segment 390 of the endovascular device is at least 60.3% of the length of the working segment 380. According to some such embodiments, the length of the support segment 390 of the endovascular device is at least 60.4% of the length of the working segment 380. According to some such embodiments, the length of the support segment 390 of the endovascular device is at least 60.5% of the length of the working segment 380. According to some such embodiments, the length of the support segment 390 of the endovascular device is at least 60.6% of the length of the working segment 380. According to some such embodiments, the length of the support segment 390 of the endovascular device is at least 60.7% of the length of the working segment 380. According to some such embodiments, the length of the support segment 390 of the endovascular device is at least 60.8% of the length of the working segment 380. According to some such embodiments, the length of the support segment 390 of the endovascular device is at least 60.9% of the length of the working segment 380. According to some such embodiments, the length of the support segment 390 of the endovascular device is at least 61% of the length of the working segment 380.

According to some such embodiments, the length of the support segment 390 of the endovascular device is at least 61.1% of the length of the working segment 380. According to some such embodiments, the length of the support segment 390 of the endovascular device is at least 61.2% of the length of the working segment 380. According to some such embodiments, the length of the support segment 390 of the endovascular device is at least 61.3% of the length of the working segment 380. According to some such embodiments, the length of the support segment 390 of the endovascular device is at least 61.4% of the length of the working segment 380. According to some such embodiments, the length of the support segment 390 of the endovascular device is at least 61.5% of the length of the working segment 380. According to some such embodiments, the length of the support segment 390 of the endovascular device is at least 61.6% of the length of the working segment 380. According to some such embodiments, the length of the support segment 390 of the endovascular device is at least 61.7% of the length of the working segment 380. According to some such embodiments, the length of the support segment 390 of the endovascular device is at least 61.8% of the length of the working segment 380. According to some such embodiments, the length of the support segment 390 of the endovascular device is at least 61.9% of the length of the working segment 380. According to some such embodiments, the length of the support segment 390 of the endovascular device is at least 62% of the length of the working segment 380.

According to some such embodiments, the length of the support segment 390 of the endovascular device is at least 62.1% of the length of the working segment 380. According to some such embodiments, the length of the support segment 390 of the endovascular device is at least 62.2% of the length of the working segment 380. According to some such embodiments, the length of the support segment 390 of the endovascular device is at least 62.3% of the length of the working segment 380. According to some such embodiments, the length of the support segment 390 of the endovascular device is at least 62.4% of the length of the working segment 380. According to some such embodiments, the length of the support segment 390 of the endovascular device is at least 62.5% of the length of the working segment 380. According to some such embodiments, the length of the support segment 390 of the endovascular device is at least 62.6% of the length of the working segment 380. According to some such embodiments, the length of the support segment 390 of the endovascular device is at least 62.7% of the length of the working segment 380. According to some such embodiments, the length of the support segment 390 of the endovascular device is at least 62.8% of the length of the working segment 380. According to some such embodiments, the length of the support segment 390 of the endovascular device is at least 62.9% of the length of the working segment 380. According to some such embodiments, the length of the support segment 390 of the endovascular device is at least 63% of the length of the working segment 380.

According to some such embodiments, the length of the support segment 390 of the endovascular device is at least 63.1% of the length of the working segment 380. According to some such embodiments, the length of the support segment 390 of the endovascular device is at least 63.2% of the length of the working segment 380. According to some such embodiments, the length of the support segment 390 of the endovascular device is at least 63.3% of the length of the working segment 380. According to some such embodiments, the length of the support segment 390 of the endovascular device is at least 63.4% of the length of the working segment 380. According to some such embodiments, the length of the support segment 390 of the endovascular device is at least 63.5% of the length of the working segment 380. According to some such embodiments, the length of the support segment 390 of the endovascular device is at least 63.6% of the length of the working segment 380. According to some such embodiments, the length of the support segment 390 of the endovascular device is at least 63.7% of the length of the working segment 380. According to some such embodiments, the length of the support segment 390 of the endovascular device is at least 63.8% of the length of the working segment 380. According to some such embodiments, the length of the support segment 390 of the endovascular device is at least 63.9% of the length of the working segment 380. According to some such embodiments, the length of the support segment 390 of the endovascular device is at least 64% of the length of the working segment 380.

According to some such embodiments, the length of the support segment 390 of the endovascular device is at least 64.1% of the length of the working segment 380. According to some such embodiments, the length of the support segment 390 of the endovascular device is at least 64.2% of the length of the working segment 380. According to some such embodiments, the length of the support segment 390 of the endovascular device is at least 64.3% of the length of the working segment 380. According to some such embodiments, the length of the support segment 390 of the endovascular device is at least 64.4% of the length of the working segment 380. According to some such embodiments, the length of the support segment 390 of the endovascular device is at least 64.5% of the length of the working segment 380. According to some such embodiments, the length of the support segment 390 of the endovascular device is at least 64.6% of the length of the working segment 380. According to some such embodiments, the length of the support segment 390 of the endovascular device is at least 64.7% of the length of the working segment 380. According to some such embodiments, the length of the support segment 390 of the endovascular device is at least 64.8% of the length of the working segment 380. According to some such embodiments, the length of the support segment 390 of the endovascular device is at least 64.9% of the length of the working segment 380. According to some such embodiments, the length of the support segment 390 of the endovascular device is at least 65% of the length of the working segment 380.

According to some such embodiments, the length of the support segment 390 of the endovascular device is at least 65.1% of the length of the working segment 380. According to some such embodiments, the length of the support segment 390 of the endovascular device is at least 65.2% of the length of the working segment 380. According to some such embodiments, the length of the support segment 390 of the endovascular device is at least 65.3% of the length of the working segment 380. According to some such embodiments, the length of the support segment 390 of the endovascular device is at least 65.4% of the length of the working segment 380. According to some such embodiments, the length of the support segment 390 of the endovascular device is at least 65.5% of the length of the working segment 380. According to some such embodiments, the length of the support segment 390 of the endovascular device is at least 65.6% of the length of the working segment 380. According to some such embodiments, the length of the support segment 390 of the endovascular device is at least 65.7% of the length of the working segment 380. According to some such embodiments, the length of the support segment 390 of the endovascular device is at least 65.8% of the length of the working segment 380. According to some such embodiments, the length of the support segment 390 of the endovascular device is at least 65.9% of the length of the working segment 380. According to some such embodiments, the length of the support segment 390 of the endovascular device is at least 66% of the length of the working segment 380.

According to some such embodiments, the length of the support segment 390 of the endovascular device is at least 66.1% of the length of the working segment 380. According to some such embodiments, the length of the support segment 390 of the endovascular device is at least 66.2% of the length of the working segment 380. According to some such embodiments, the length of the support segment 390 of the endovascular device is at least 66.3% of the length of the working segment 380. According to some such embodiments, the length of the support segment 390 of the endovascular device is at least 66.4% of the length of the working segment 380. According to some such embodiments, the length of the support segment 390 of the endovascular device is at least 66.5% of the length of the working segment 380. According to some such embodiments, the length of the support segment 390 of the endovascular device is at least 66.6% of the length of the working segment 380. According to some such embodiments, the length of the support segment 390 of the endovascular device is at least 66.7% of the length of the working segment 380. According to some such embodiments, the length of the support segment 390 of the endovascular device is at least 66.8% of the length of the working segment 380. According to some such embodiments, the length of the support segment 390 of the endovascular device is at least 66.9% of the length of the working segment 380. According to some such embodiments, the length of the support segment 390 of the endovascular device is at least 67% of the length of the working segment 380.

According to some such embodiments, the length of the support segment 390 of the endovascular device is at least 67.1% of the length of the working segment 380. According to some such embodiments, the length of the support segment 390 of the endovascular device is at least 67.2% of the length of the working segment 380. According to some such embodiments, the length of the support segment 390 of the endovascular device is at least 67.3% of the length of the working segment 380. According to some such embodiments, the length of the support segment 390 of the endovascular device is at least 67.4% of the length of the working segment 380. According to some such embodiments, the length of the support segment 390 of the endovascular device is at least 67.5% of the length of the working segment 380. According to some such embodiments, the length of the support segment 390 of the endovascular device is at least 67.6% of the length of the working segment 380. According to some such embodiments, the length of the support segment 390 of the endovascular device is at least 67.7% of the length of the working segment 380. According to some such embodiments, the length of the support segment 390 of the endovascular device is at least 67.8% of the length of the working segment 380. According to some such embodiments, the length of the support segment 390 of the endovascular device is at least 67.9% of the length of the working segment 380. According to some such embodiments, the length of the support segment 390 of the endovascular device is at least 68% of the length of the working segment 380.

According to some such embodiments, the length of the support segment 390 of the endovascular device is at least 68.1% of the length of the working segment 380. According to some such embodiments, the length of the support segment 390 of the endovascular device is at least 68.2% of the length of the working segment 380. According to some such embodiments, the length of the support segment 390 of the endovascular device is at least 68.3% of the length of the working segment 380. According to some such embodiments, the length of the support segment 390 of the endovascular device is at least 68.4% of the length of the working segment 380. According to some such embodiments, the length of the support segment 390 of the endovascular device is at least 68.5% of the length of the working segment 380. According to some such embodiments, the length of the support segment 390 of the endovascular device is at least 68.6% of the length of the working segment 380. According to some such embodiments, the length of the support segment 390 of the endovascular device is at least 68.7% of the length of the working segment 380. According to some such embodiments, the length of the support segment 390 of the endovascular device is at least 68.8% of the length of the working segment 380. According to some such embodiments, the length of the support segment 390 of the endovascular device is at least 68.9% of the length of the working segment 380. According to some such embodiments, the length of the support segment 390 of the endovascular device is at least 69% of the length of the working segment 380.

According to some such embodiments, the length of the support segment 390 of the endovascular device is at least 69.1% of the length of the working segment 380. According to some such embodiments, the length of the support segment 390 of the endovascular device is at least 69.2% of the length of the working segment 380. According to some such embodiments, the length of the support segment 390 of the endovascular device is at least 69.3% of the length of the working segment 380. According to some such embodiments, the length of the support segment 390 of the endovascular device is at least 69.4% of the length of the working segment 380. According to some such embodiments, the length of the support segment 390 of the endovascular device is at least 69.5% of the length of the working segment 380. According to some such embodiments, the length of the support segment 390 of the endovascular device is at least 69.6% of the length of the working segment 380. According to some such embodiments, the length of the support segment 390 of the endovascular device is at least 69.7% of the length of the working segment 380. According to some such embodiments, the length of the support segment 390 of the endovascular device is at least 69.8% of the length of the working segment 380. According to some such embodiments, the length of the support segment 390 of the endovascular device is at least 69.9% of the length of the working segment 380. According to some such embodiments, the length of the support segment 390 of the endovascular device is at least 70% of the length of the working segment 380.

According to some such embodiments, the length of the support segment 390 of the endovascular device is at least 70.1% of the length of the working segment 380. According to some such embodiments, the length of the support segment 390 of the endovascular device is at least 70.2% of the length of the working segment 380. According to some such embodiments, the length of the support segment 390 of the endovascular device is at least 70.3% of the length of the working segment 380. According to some such embodiments, the length of the support segment 390 of the endovascular device is at least 70.4% of the length of the working segment 380. According to some such embodiments, the length of the support segment 390 of the endovascular device is at least 70.5% of the length of the working segment 380. According to some such embodiments, the length of the support segment 390 of the endovascular device is at least 70.6% of the length of the working segment 380. According to some such embodiments, the length of the support segment 390 of the endovascular device is at least 70.7% of the length of the working segment 380. According to some such embodiments, the length of the support segment 390 of the endovascular device is at least 70.8% of the length of the working segment 380. According to some such embodiments, the length of the support segment 390 of the endovascular device is at least 70.9% of the length of the working segment 380. According to some such embodiments, the length of the support segment 390 of the endovascular device is at least 71% of the length of the working segment 380.

According to some such embodiments, the length of the support segment 390 of the endovascular device is at least 71.1% of the length of the working segment 380. According to some such embodiments, the length of the support segment 390 of the endovascular device is at least 71.2% of the length of the working segment 380. According to some such embodiments, the length of the support segment 390 of the endovascular device is at least 71.3% of the length of the working segment 380. According to some such embodiments, the length of the support segment 390 of the endovascular device is at least 71.4% of the length of the working segment 380. According to some such embodiments, the length of the support segment 390 of the endovascular device is at least 71.5% of the length of the working segment 380. According to some such embodiments, the length of the support segment 390 of the endovascular device is at least 71.6% of the length of the working segment 380. According to some such embodiments, the length of the support segment 390 of the endovascular device is at least 71.7% of the length of the working segment 380. According to some such embodiments, the length of the support segment 390 of the endovascular device is at least 71.8% of the length of the working segment 380. According to some such embodiments, the length of the support segment 390 of the endovascular device is at least 71.9% of the length of the working segment 380. According to some such embodiments, the length of the support segment 390 of the endovascular device is at least 72% of the length of the working segment 380.

According to some such embodiments, the length of the support segment 390 of the endovascular device is at least 72.1% of the length of the working segment 380. According to some such embodiments, the length of the support segment 390 of the endovascular device is at least 72.2% of the length of the working segment 380. According to some such embodiments, the length of the support segment 390 of the endovascular device is at least 72.3% of the length of the working segment 380. According to some such embodiments, the length of the support segment 390 of the endovascular device is at least 72.4% of the length of the working segment 380. According to some such embodiments, the length of the support segment 390 of the endovascular device is at least 72.5% of the length of the working segment 380. According to some such embodiments, the length of the support segment 390 of the endovascular device is at least 72.6% of the length of the working segment 380. According to some such embodiments, the length of the support segment 390 of the endovascular device is at least 72.7% of the length of the working segment 380. According to some such embodiments, the length of the support segment 390 of the endovascular device is at least 72.8% of the length of the working segment 380. According to some such embodiments, the length of the support segment 390 of the endovascular device is at least 72.9% of the length of the working segment 380. According to some such embodiments, the length of the support segment 390 of the endovascular device is at least 73% of the length of the working segment 380.

According to some such embodiments, the length of the support segment 390 of the endovascular device is at least 73.1% of the length of the working segment 380. According to some such embodiments, the length of the support segment 390 of the endovascular device is at least 73.2% of the length of the working segment 380. According to some such embodiments, the length of the support segment 390 of the endovascular device is at least 73.3% of the length of the working segment 380. According to some such embodiments, the length of the support segment 390 of the endovascular device is at least 73.4% of the length of the working segment 380. According to some such embodiments, the length of the support segment 390 of the endovascular device is at least 73.5% of the length of the working segment 380. According to some such embodiments, the length of the support segment 390 of the endovascular device is at least 73.6% of the length of the working segment 380. According to some such embodiments, the length of the support segment 390 of the endovascular device is at least 73.7% of the length of the working segment 380. According to some such embodiments, the length of the support segment 390 of the endovascular device is at least 73.8% of the length of the working segment 380. According to some such embodiments, the length of the support segment 390 of the endovascular device is at least 73.9% of the length of the working segment 380. According to some such embodiments, the length of the support segment 390 of the endovascular device is at least 74% of the length of the working segment 380.

According to some such embodiments, the length of the support segment 390 of the endovascular device is at least 74.1% of the length of the working segment 380. According to some such embodiments, the length of the support segment 390 of the endovascular device is at least 74.2% of the length of the working segment 380. According to some such embodiments, the length of the support segment 390 of the endovascular device is at least 74.3% of the length of the working segment 380. According to some such embodiments, the length of the support segment 390 of the endovascular device is at least 74.4% of the length of the working segment 380. According to some such embodiments, the length of the support segment 390 of the endovascular device is at least 74.5% of the length of the working segment 380. According to some such embodiments, the length of the support segment 390 of the endovascular device is at least 74.6% of the length of the working segment 380. According to some such embodiments, the length of the support segment 390 of the endovascular device is at least 74.7% of the length of the working segment 380. According to some such embodiments, the length of the support segment 390 of the endovascular device is at least 74.8% of the length of the working segment 380. According to some such embodiments, the length of the support segment 390 of the endovascular device is at least 74.9% of the length of the working segment 380. According to some such embodiments, the length of the support segment 390 of the endovascular device is at least 75% of the length of the working segment 380.

According to some such embodiments, the length of the support segment 390 of the endovascular device is at least 75.1% of the length of the working segment 380. According to some such embodiments, the length of the support segment 390 of the endovascular device is at least 75.2% of the length of the working segment 380. According to some such embodiments, the length of the support segment 390 of the endovascular device is at least 75.3% of the length of the working segment 380. According to some such embodiments, the length of the support segment 390 of the endovascular device is at least 75.4% of the length of the working segment 380. According to some such embodiments, the length of the support segment 390 of the endovascular device is at least 75.5% of the length of the working segment 380. According to some such embodiments, the length of the support segment 390 of the endovascular device is at least 75.6% of the length of the working segment 380. According to some such embodiments, the length of the support segment 390 of the endovascular device is at least 75.7% of the length of the working segment 380. According to some such embodiments, the length of the support segment 390 of the endovascular device is at least 75.8% of the length of the working segment 380. According to some such embodiments, the length of the support segment 390 of the endovascular device is at least 75.9% of the length of the working segment 380. According to some such embodiments, the length of the support segment 390 of the endovascular device is at least 76% of the length of the working segment 380.

According to some such embodiments, the length of the support segment 390 of the endovascular device is at least 76.1% of the length of the working segment 380. According to some such embodiments, the length of the support segment 390 of the endovascular device is at least 76.2% of the length of the working segment 380. According to some such embodiments, the length of the support segment 390 of the endovascular device is at least 76.3% of the length of the working segment 380. According to some such embodiments, the length of the support segment 390 of the endovascular device is at least 76.4% of the length of the working segment 380. According to some such embodiments, the length of the support segment 390 of the endovascular device is at least 76.5% of the length of the working segment 380. According to some such embodiments, the length of the support segment 390 of the endovascular device is at least 76.6% of the length of the working segment 380. According to some such embodiments, the length of the support segment 390 of the endovascular device is at least 76.7% of the length of the working segment 380. According to some such embodiments, the length of the support segment 390 of the endovascular device is at least 76.8% of the length of the working segment 380. According to some such embodiments, the length of the support segment 390 of the endovascular device is at least 76.9% of the length of the working segment 380. According to some such embodiments, the length of the support segment 390 of the endovascular device is at least 77% of the length of the working segment 380.

According to some such embodiments, the length of the support segment 390 of the endovascular device is at least 77.1% of the length of the working segment 380. According to some such embodiments, the length of the support segment 390 of the endovascular device is at least 77.2% of the length of the working segment 380. According to some such embodiments, the length of the support segment 390 of the endovascular device is at least 77.3% of the length of the working segment 380. According to some such embodiments, the length of the support segment 390 of the endovascular device is at least 77.4% of the length of the working segment 380. According to some such embodiments, the length of the support segment 390 of the endovascular device is at least 77.5% of the length of the working segment 380. According to some such embodiments, the length of the support segment 390 of the endovascular device is at least 77.6% of the length of the working segment 380. According to some such embodiments, the length of the support segment 390 of the endovascular device is at least 77.7% of the length of the working segment 380. According to some such embodiments, the length of the support segment 390 of the endovascular device is at least 77.8% of the length of the working segment 380. According to some such embodiments, the length of the support segment 390 of the endovascular device is at least 77.9% of the length of the working segment 380. According to some such embodiments, the length of the support segment 390 of the endovascular device is at least 78% of the length of the working segment 380.

According to some such embodiments, the length of the support segment 390 of the endovascular device is at least 78.1% of the length of the working segment 380. According to some such embodiments, the length of the support segment 390 of the endovascular device is at least 78.2% of the length of the working segment 380. According to some such embodiments, the length of the support segment 390 of the endovascular device is at least 78.3% of the length of the working segment 380. According to some such embodiments, the length of the support segment 390 of the endovascular device is at least 78.4% of the length of the working segment 380. According to some such embodiments, the length of the support segment 390 of the endovascular device is at least 78.5% of the length of the working segment 380. According to some such embodiments, the length of the support segment 390 of the endovascular device is at least 78.6% of the length of the working segment 380. According to some such embodiments, the length of the support segment 390 of the endovascular device is at least 78.7% of the length of the working segment 380. According to some such embodiments, the length of the support segment 390 of the endovascular device is at least 78.8% of the length of the working segment 380. According to some such embodiments, the length of the support segment 390 of the endovascular device is at least 78.9% of the length of the working segment 380. According to some such embodiments, the length of the support segment 390 of the endovascular device is at least 79% of the length of the working segment 380.

According to some such embodiments, the length of the support segment 390 of the endovascular device is at least 79.1% of the length of the working segment 380. According to some such embodiments, the length of the support segment 390 of the endovascular device is at least 79.2% of the length of the working segment 380. According to some such embodiments, the length of the support segment 390 of the endovascular device is at least 79.3% of the length of the working segment 380. According to some such embodiments, the length of the support segment 390 of the endovascular device is at least 79.4% of the length of the working segment 380. According to some such embodiments, the length of the support segment 390 of the endovascular device is at least 79.5% of the length of the working segment 380. According to some such embodiments, the length of the support segment 390 of the endovascular device is at least 79.6% of the length of the working segment 380. According to some such embodiments, the length of the support segment 390 of the endovascular device is at least 79.7% of the length of the working segment 380. According to some such embodiments, the length of the support segment 390 of the endovascular device is at least 79.8% of the length of the working segment 380. According to some such embodiments, the length of the support segment 390 of the endovascular device is at least 79.9% of the length of the working segment 380. According to some such embodiments, the length of the support segment 390 of the endovascular device is at least 80% of the length of the working segment 380.

According to some such embodiments, the length of the support segment 390 of the endovascular device is at least 80.1% of the length of the working segment 380. According to some such embodiments, the length of the support segment 390 of the endovascular device is at least 80.2% of the length of the working segment 380. According to some such embodiments, the length of the support segment 390 of the endovascular device is at least 80.3% of the length of the working segment 380. According to some such embodiments, the length of the support segment 390 of the endovascular device is at least 80.4% of the length of the working segment 380. According to some such embodiments, the length of the support segment 390 of the endovascular device is at least 80.5% of the length of the working segment 380. According to some such embodiments, the length of the support segment 390 of the endovascular device is at least 80.6% of the length of the working segment 380. According to some such embodiments, the length of the support segment 390 of the endovascular device is at least 80.7% of the length of the working segment 380. According to some such embodiments, the length of the support segment 390 of the endovascular device is at least 80.8% of the length of the working segment 380. According to some such embodiments, the length of the support segment 390 of the endovascular device is at least 80.9% of the length of the working segment 380. According to some such embodiments, the length of the support segment 390 of the endovascular device is at least 81% of the length of the working segment 380.

According to some such embodiments, the length of the support segment 390 of the endovascular device is at least 81.1% of the length of the working segment 380. According to some such embodiments, the length of the support segment 390 of the endovascular device is at least 81.2% of the length of the working segment 380. According to some such embodiments, the length of the support segment 390 of the endovascular device is at least 81.3% of the length of the working segment 380. According to some such embodiments, the length of the support segment 390 of the endovascular device is at least 81.4% of the length of the working segment 380. According to some such embodiments, the length of the support segment 390 of the endovascular device is at least 81.5% of the length of the working segment 380. According to some such embodiments, the length of the support segment 390 of the endovascular device is at least 81.6% of the length of the working segment 380. According to some such embodiments, the length of the support segment 390 of the endovascular device is at least 81.7% of the length of the working segment 380. According to some such embodiments, the length of the support segment 390 of the endovascular device is at least 81.8% of the length of the working segment 380. According to some such embodiments, the length of the support segment 390 of the endovascular device is at least 81.9% of the length of the working segment 380. According to some such embodiments, the length of the support segment 390 of the endovascular device is at least 82% of the length of the working segment 380.

According to some such embodiments, the length of the support segment 390 of the endovascular device is at least 82.1% of the length of the working segment 380. According to some such embodiments, the length of the support segment 390 of the endovascular device is at least 82.2% of the length of the working segment 380. According to some such embodiments, the length of the support segment 390 of the endovascular device is at least 82.3% of the length of the working segment 380. According to some such embodiments, the length of the support segment 390 of the endovascular device is at least 82.4% of the length of the working segment 380. According to some such embodiments, the length of the support segment 390 of the endovascular device is at least 82.5% of the length of the working segment 380. According to some such embodiments, the length of the support segment 390 of the endovascular device is at least 82.6% of the length of the working segment 380. According to some such embodiments, the length of the support segment 390 of the endovascular device is at least 82.7% of the length of the working segment 380. According to some such embodiments, the length of the support segment 390 of the endovascular device is at least 82.8% of the length of the working segment 380. According to some such embodiments, the length of the support segment 390 of the endovascular device is at least 82.9% of the length of the working segment 380. According to some such embodiments, the length of the support segment 390 of the endovascular device is at least 83% of the length of the working segment 380.

According to some such embodiments, the length of the support segment 390 of the endovascular device is at least 83.1% of the length of the working segment 380. According to some such embodiments, the length of the support segment 390 of the endovascular device is at least 83.2% of the length of the working segment 380. According to some such embodiments, the length of the support segment 390 of the endovascular device is at least 83.3% of the length of the working segment 380. According to some such embodiments, the length of the support segment 390 of the endovascular device is at least 83.4% of the length of the working segment 380. According to some such embodiments, the length of the support segment 390 of the endovascular device is at least 83.5% of the length of the working segment 380. According to some such embodiments, the length of the support segment 390 of the endovascular device is at least 83.6% of the length of the working segment 380. According to some such embodiments, the length of the support segment 390 of the endovascular device is at least 83.7% of the length of the working segment 380. According to some such embodiments, the length of the support segment 390 of the endovascular device is at least 83.8% of the length of the working segment 380. According to some such embodiments, the length of the support segment 390 of the endovascular device is at least 83.9% of the length of the working segment 380. According to some such embodiments, the length of the support segment 390 of the endovascular device is at least 84% of the length of the working segment 380.

According to some such embodiments, the length of the support segment 390 of the endovascular device is at least 84.1% of the length of the working segment 380. According to some such embodiments, the length of the support segment 390 of the endovascular device is at least 84.2% of the length of the working segment 380. According to some such embodiments, the length of the support segment 390 of the endovascular device is at least 84.3% of the length of the working segment 380. According to some such embodiments, the length of the support segment 390 of the endovascular device is at least 84.4% of the length of the working segment 380. According to some such embodiments, the length of the support segment 390 of the endovascular device is at least 84.5% of the length of the working segment 380. According to some such embodiments, the length of the support segment 390 of the endovascular device is at least 84.6% of the length of the working segment 380. According to some such embodiments, the length of the support segment 390 of the endovascular device is at least 84.7% of the length of the working segment 380. According to some such embodiments, the length of the support segment 390 of the endovascular device is at least 84.8% of the length of the working segment 380. According to some such embodiments, the length of the support segment 390 of the endovascular device is at least 84.9% of the length of the working segment 380. According to some such embodiments, the length of the support segment 390 of the endovascular device is at least 85% of the length of the working segment 380.

According to some such embodiments, the length of the support segment 390 of the endovascular device is at least 85.1% of the length of the working segment 380. According to some such embodiments, the length of the support segment 390 of the endovascular device is at least 85.2% of the length of the working segment 380. According to some such embodiments, the length of the support segment 390 of the endovascular device is at least 85.3% of the length of the working segment 380. According to some such embodiments, the length of the support segment 390 of the endovascular device is at least 85.4% of the length of the working segment 380. According to some such embodiments, the length of the support segment 390 of the endovascular device is at least 85.5% of the length of the working segment 380. According to some such embodiments, the length of the support segment 390 of the endovascular device is at least 85.6% of the length of the working segment 380. According to some such embodiments, the length of the support segment 390 of the endovascular device is at least 85.7% of the length of the working segment 380. According to some such embodiments, the length of the support segment 390 of the endovascular device is at least 85.8% of the length of the working segment 380. According to some such embodiments, the length of the support segment 390 of the endovascular device is at least 85.9% of the length of the working segment 380. According to some such embodiments, the length of the support segment 390 of the endovascular device is at least 86% of the length of the working segment 380.

According to some such embodiments, the length of the support segment 390 of the endovascular device is at least 86.1% of the length of the working segment 380. According to some such embodiments, the length of the support segment 390 of the endovascular device is at least 86.2% of the length of the working segment 380. According to some such embodiments, the length of the support segment 390 of the endovascular device is at least 86.3% of the length of the working segment 380. According to some such embodiments, the length of the support segment 390 of the endovascular device is at least 86.4% of the length of the working segment 380. According to some such embodiments, the length of the support segment 390 of the endovascular device is at least 86.5% of the length of the working segment 380. According to some such embodiments, the length of the support segment 390 of the endovascular device is at least 86.6% of the length of the working segment 380. According to some such embodiments, the length of the support segment 390 of the endovascular device is at least 86.7% of the length of the working segment 380. According to some such embodiments, the length of the support segment 390 of the endovascular device is at least 86.8% of the length of the working segment 380. According to some such embodiments, the length of the support segment 390 of the endovascular device is at least 86.9% of the length of the working segment 380. According to some such embodiments, the length of the support segment 390 of the endovascular device is at least 87% of the length of the working segment 380.

According to some such embodiments, the length of the support segment 390 of the endovascular device is at least 87.1% of the length of the working segment 380. According to some such embodiments, the length of the support segment 390 of the endovascular device is at least 87.2% of the length of the working segment 380. According to some such embodiments, the length of the support segment 390 of the endovascular device is at least 87.3% of the length of the working segment 380. According to some such embodiments, the length of the support segment 390 of the endovascular device is at least 87.4% of the length of the working segment 380. According to some such embodiments, the length of the support segment 390 of the endovascular device is at least 87.5% of the length of the working segment 380. According to some such embodiments, the length of the support segment 390 of the endovascular device is at least 87.6% of the length of the working segment 380. According to some such embodiments, the length of the support segment 390 of the endovascular device is at least 87.7% of the length of the working segment 380. According to some such embodiments, the length of the support segment 390 of the endovascular device is at least 87.8% of the length of the working segment 380. According to some such embodiments, the length of the support segment 390 of the endovascular device is at least 87.9% of the length of the working segment 380. According to some such embodiments, the length of the support segment 390 of the endovascular device is at least 88% of the length of the working segment 380.

According to some such embodiments, the length of the support segment 390 of the endovascular device is at least 88.1% of the length of the working segment 380. According to some such embodiments, the length of the support segment 390 of the endovascular device is at least 88.2% of the length of the working segment 380. According to some such embodiments, the length of the support segment 390 of the endovascular device is at least 88.3% of the length of the working segment 380. According to some such embodiments, the length of the support segment 390 of the endovascular device is at least 88.4% of the length of the working segment 380. According to some such embodiments, the length of the support segment 390 of the endovascular device is at least 88.5% of the length of the working segment 380. According to some such embodiments, the length of the support segment 390 of the endovascular device is at least 88.6% of the length of the working segment 380. According to some such embodiments, the length of the support segment 390 of the endovascular device is at least 88.7% of the length of the working segment 380. According to some such embodiments, the length of the support segment 390 of the endovascular device is at least 88.8% of the length of the working segment 380. According to some such embodiments, the length of the support segment 390 of the endovascular device is at least 88.9% of the length of the working segment 380. According to some such embodiments, the length of the support segment 390 of the endovascular device is at least 89% of the length of the working segment 380.

According to some such embodiments, the length of the support segment 390 of the endovascular device is at least 89.1% of the length of the working segment 380. According to some such embodiments, the length of the support segment 390 of the endovascular device is at least 89.2% of the length of the working segment 380. According to some such embodiments, the length of the support segment 390 of the endovascular device is at least 89.3% of the length of the working segment 380. According to some such embodiments, the length of the support segment 390 of the endovascular device is at least 89.4% of the length of the working segment 380. According to some such embodiments, the length of the support segment 390 of the endovascular device is at least 89.5% of the length of the working segment 380. According to some such embodiments, the length of the support segment 390 of the endovascular device is at least 89.6% of the length of the working segment 380. According to some such embodiments, the length of the support segment 390 of the endovascular device is at least 89.7% of the length of the working segment 380. According to some such embodiments, the length of the support segment 390 of the endovascular device is at least 89.8% of the length of the working segment 380. According to some such embodiments, the length of the support segment 390 of the endovascular device is at least 89.9% of the length of the working segment 380. According to some such embodiments, the length of the support segment 390 of the endovascular device is at least 90% of the length of the working segment 380.

According to some such embodiments, the length of the support segment 390 of the endovascular device is at least 90.1% of the length of the working segment 380. According to some such embodiments, the length of the support segment 390 of the endovascular device is at least 90.2% of the length of the working segment 380. According to some such embodiments, the length of the support segment 390 of the endovascular device is at least 90.3% of the length of the working segment 380. According to some such embodiments, the length of the support segment 390 of the endovascular device is at least 90.4% of the length of the working segment 380. According to some such embodiments, the length of the support segment 390 of the endovascular device is at least 90.5% of the length of the working segment 380. According to some such embodiments, the length of the support segment 390 of the endovascular device is at least 90.6% of the length of the working segment 380. According to some such embodiments, the length of the support segment 390 of the endovascular device is at least 90.7% of the length of the working segment 380. According to some such embodiments, the length of the support segment 390 of the endovascular device is at least 90.8% of the length of the working segment 380. According to some such embodiments, the length of the support segment 390 of the endovascular device is at least 90.9% of the length of the working segment 380. According to some such embodiments, the length of the support segment 390 of the endovascular device is at least 91% of the length of the working segment 380.

According to some such embodiments, the length of the support segment 390 of the endovascular device is at least 91.1% of the length of the working segment 380. According to some such embodiments, the length of the support segment 390 of the endovascular device is at least 91.2% of the length of the working segment 380. According to some such embodiments, the length of the support segment 390 of the endovascular device is at least 91.3% of the length of the working segment 380. According to some such embodiments, the length of the support segment 390 of the endovascular device is at least 91.4% of the length of the working segment 380. According to some such embodiments, the length of the support segment 390 of the endovascular device is at least 91.5% of the length of the working segment 380. According to some such embodiments, the length of the support segment 390 of the endovascular device is at least 91.6% of the length of the working segment 380. According to some such embodiments, the length of the support segment 390 of the endovascular device is at least 91.7% of the length of the working segment 380. According to some such embodiments, the length of the support segment 390 of the endovascular device is at least 91.8% of the length of the working segment 380. According to some such embodiments, the length of the support segment 390 of the endovascular device is at least 91.9% of the length of the working segment 380. According to some such embodiments, the length of the support segment 390 of the endovascular device is at least 92% of the length of the working segment 380.

According to some such embodiments, the length of the support segment 390 of the endovascular device is at least 92.1% of the length of the working segment 380. According to some such embodiments, the length of the support segment 390 of the endovascular device is at least 92.2% of the length of the working segment 380. According to some such embodiments, the length of the support segment 390 of the endovascular device is at least 92.3% of the length of the working segment 380. According to some such embodiments, the length of the support segment 390 of the endovascular device is at least 92.4% of the length of the working segment 380. According to some such embodiments, the length of the support segment 390 of the endovascular device is at least 92.5% of the length of the working segment 380. According to some such embodiments, the length of the support segment 390 of the endovascular device is at least 92.6% of the length of the working segment 380. According to some such embodiments, the length of the support segment 390 of the endovascular device is at least 92.7% of the length of the working segment 380. According to some such embodiments, the length of the support segment 390 of the endovascular device is at least 92.8% of the length of the working segment 380. According to some such embodiments, the length of the support segment 390 of the endovascular device is at least 92.9% of the length of the working segment 380. According to some such embodiments, the length of the support segment 390 of the endovascular device is at least 93% of the length of the working segment 380.

According to some such embodiments, the length of the support segment 390 of the endovascular device is at least 93.1% of the length of the working segment 380. According to some such embodiments, the length of the support segment 390 of the endovascular device is at least 93.2% of the length of the working segment 380. According to some such embodiments, the length of the support segment 390 of the endovascular device is at least 93.3% of the length of the working segment 380. According to some such embodiments, the length of the support segment 390 of the endovascular device is at least 93.4% of the length of the working segment 380. According to some such embodiments, the length of the support segment 390 of the endovascular device is at least 93.5% of the length of the working segment 380. According to some such embodiments, the length of the support segment 390 of the endovascular device is at least 93.6% of the length of the working segment 380. According to some such embodiments, the length of the support segment 390 of the endovascular device is at least 93.7% of the length of the working segment 380. According to some such embodiments, the length of the support segment 390 of the endovascular device is at least 93.8% of the length of the working segment 380. According to some such embodiments, the length of the support segment 390 of the endovascular device is at least 93.9% of the length of the working segment 380. According to some such embodiments, the length of the support segment 390 of the endovascular device is at least 94% of the length of the working segment 380.

According to some such embodiments, the length of the support segment 390 of the endovascular device is at least 94.1% of the length of the working segment 380. According to some such embodiments, the length of the support segment 390 of the endovascular device is at least 94.2% of the length of the working segment 380. According to some such embodiments, the length of the support segment 390 of the endovascular device is at least 94.3% of the length of the working segment 380. According to some such embodiments, the length of the support segment 390 of the endovascular device is at least 94.4% of the length of the working segment 380. According to some such embodiments, the length of the support segment 390 of the endovascular device is at least 94.5% of the length of the working segment 380. According to some such embodiments, the length of the support segment 390 of the endovascular device is at least 94.6% of the length of the working segment 380. According to some such embodiments, the length of the support segment 390 of the endovascular device is at least 94.7% of the length of the working segment 380. According to some such embodiments, the length of the support segment 390 of the endovascular device is at least 94.8% of the length of the working segment 380. According to some such embodiments, the length of the support segment 390 of the endovascular device is at least 94.9% of the length of the working segment 380. According to some such embodiments, the length of the support segment 390 of the endovascular device is at least 95% of the length of the working segment 380.

According to some such embodiments, the length of the support segment 390 of the endovascular device is at least 95.1% of the length of the working segment 380. According to some such embodiments, the length of the support segment 390 of the endovascular device is at least 95.2% of the length of the working segment 380. According to some such embodiments, the length of the support segment 390 of the endovascular device is at least 95.3% of the length of the working segment 380. According to some such embodiments, the length of the support segment 390 of the endovascular device is at least 95.4% of the length of the working segment 380. According to some such embodiments, the length of the support segment 390 of the endovascular device is at least 95.5% of the length of the working segment 380. According to some such embodiments, the length of the support segment 390 of the endovascular device is at least 95.6% of the length of the working segment 380. According to some such embodiments, the length of the support segment 390 of the endovascular device is at least 95.7% of the length of the working segment 380. According to some such embodiments, the length of the support segment 390 of the endovascular device is at least 95.8% of the length of the working segment 380. According to some such embodiments, the length of the support segment 390 of the endovascular device is at least 95.9% of the length of the working segment 380. According to some such embodiments, the length of the support segment 390 of the endovascular device is at least 96% of the length of the working segment 380.

According to some such embodiments, the length of the support segment 390 of the endovascular device is at least 96.1% of the length of the working segment 380. According to some such embodiments, the length of the support segment 390 of the endovascular device is at least 96.2% of the length of the working segment 380. According to some such embodiments, the length of the support segment 390 of the endovascular device is at least 96.3% of the length of the working segment 380. According to some such embodiments, the length of the support segment 390 of the endovascular device is at least 96.4% of the length of the working segment 380. According to some such embodiments, the length of the support segment 390 of the endovascular device is at least 96.5% of the length of the working segment 380. According to some such embodiments, the length of the support segment 390 of the endovascular device is at least 96.6% of the length of the working segment 380. According to some such embodiments, the length of the support segment 390 of the endovascular device is at least 96.7% of the length of the working segment 380. According to some such embodiments, the length of the support segment 390 of the endovascular device is at least 96.8% of the length of the working segment 380. According to some such embodiments, the length of the support segment 390 of the endovascular device is at least 96.9% of the length of the working segment 380. According to some such embodiments, the length of the support segment 390 of the endovascular device is at least 97% of the length of the working segment 380.

According to some such embodiments, the length of the support segment 390 of the endovascular device is at least 97.1% of the length of the working segment 380. According to some such embodiments, the length of the support segment 390 of the endovascular device is at least 97.2% of the length of the working segment 380. According to some such embodiments, the length of the support segment 390 of the endovascular device is at least 97.3% of the length of the working segment 380. According to some such embodiments, the length of the support segment 390 of the endovascular device is at least 97.4% of the length of the working segment 380. According to some such embodiments, the length of the support segment 390 of the endovascular device is at least 97.5% of the length of the working segment 380. According to some such embodiments, the length of the support segment 390 of the endovascular device is at least 97.6% of the length of the working segment 380. According to some such embodiments, the length of the support segment 390 of the endovascular device is at least 97.7% of the length of the working segment 380. According to some such embodiments, the length of the support segment 390 of the endovascular device is at least 97.8% of the length of the working segment 380. According to some such embodiments, the length of the support segment 390 of the endovascular device is at least 97.9% of the length of the working segment 380. According to some such embodiments, the length of the support segment 390 of the endovascular device is at least 98% of the length of the working segment 380.

According to some such embodiments, the length of the support segment 390 of the endovascular device is at least 98.1% of the length of the working segment 380. According to some such embodiments, the length of the support segment 390 of the endovascular device is at least 98.2% of the length of the working segment 380. According to some such embodiments, the length of the support segment 390 of the endovascular device is at least 98.3% of the length of the working segment 380. According to some such embodiments, the length of the support segment 390 of the endovascular device is at least 98.4% of the length of the working segment 380. According to some such embodiments, the length of the support segment 390 of the endovascular device is at least 98.5% of the length of the working segment 380. According to some such embodiments, the length of the support segment 390 of the endovascular device is at least 98.6% of the length of the working segment 380. According to some such embodiments, the length of the support segment 390 of the endovascular device is at least 98.7% of the length of the working segment 380. According to some such embodiments, the length of the support segment 390 of the endovascular device is at least 98.8% of the length of the working segment 380. According to some such embodiments, the length of the support segment 390 of the endovascular device is at least 98.9% of the length of the working segment 380. According to some such embodiments, the length of the support segment 390 of the endovascular device is at least 99% of the length of the working segment 380.

According to some such embodiments, the length of the support segment 390 of the endovascular device is at least 99.1% of the length of the working segment 380. According to some such embodiments, the length of the support segment 390 of the endovascular device is at least 99.2% of the length of the working segment 380. According to some such embodiments, the length of the support segment 390 of the endovascular device is at least 99.3% of the length of the working segment 380. According to some such embodiments, the length of the support segment 390 of the endovascular device is at least 99.4% of the length of the working segment 380. According to some such embodiments, the length of the support segment 390 of the endovascular device is at least 99.5% of the length of the working segment 380. According to some such embodiments, the length of the support segment 390 of the endovascular device is at least 99.6% of the length of the working segment 380. According to some such embodiments, the length of the support segment 390 of the endovascular device is at least 99.7% of the length of the working segment 380. According to some such embodiments, the length of the support segment 390 of the endovascular device is at least 99.8% of the length of the working segment 380. According to some such embodiments, the length of the support segment 390 of the endovascular device is at least 99.9% of the length of the working segment 380. According to some such embodiments, the length of the support segment 390 of the endovascular device is at least 100% of the length of the working segment 380.

According to some such embodiments, the length of the support segment 390 of the endovascular device is at least 100.1% of the length of the working segment 380. According to some such embodiments, the length of the support segment 390 of the endovascular device is at least 100.2% of the length of the working segment 380. According to some such embodiments, the length of the support segment 390 of the endovascular device is at least 100.3% of the length of the working segment 380. According to some such embodiments, the length of the support segment 390 of the endovascular device is at least 100.4% of the length of the working segment 380. According to some such embodiments, the length of the support segment 390 of the endovascular device is at least 100.5% of the length of the working segment 380. According to some such embodiments, the length of the support segment 390 of the endovascular device is at least 100.6% of the length of the working segment 380. According to some such embodiments, the length of the support segment 390 of the endovascular device is at least 100.7% of the length of the working segment 380. According to some such embodiments, the length of the support segment 390 of the endovascular device is at least 100.8% of the length of the working segment 380. According to some such embodiments, the length of the support segment 390 of the endovascular device is at least 100.9% of the length of the working segment 380. According to some such embodiments, the length of the support segment 390 of the endovascular device is at least 101% of the length of the working segment 380. According to some such embodiments, the length of the support segment 390 of the endovascular device is at least 101.1% of the length of the working segment 380. According to some such embodiments, the length of the support segment 390 of the endovascular device is at least 101.2% of the length of the working segment 380. According to some such embodiments, the length of the support segment 390 of the endovascular device is at least 101.3% of the length of the working segment 380. According to some such embodiments, the length of the support segment 390 of the endovascular device is at least 101.4% of the length of the working segment 380. According to some such embodiments, the length of the support segment 390 of the endovascular device is at least 101.5% of the length of the working segment 380. According to some such embodiments, the length of the support segment 390 of the endovascular device is at least 101.6% of the length of the working segment 380. According to some such embodiments, the length of the support segment 390 of the endovascular device is at least 101.7% of the length of the working segment 380. According to some such embodiments, the length of the support segment 390 of the endovascular device is at least 101.8% of the length of the working segment 380. According to some such embodiments, the length of the support segment 390 of the endovascular device is at least 101.9% of the length of the working segment 380. According to some such embodiments, the length of the support segment 390 of the endovascular device is at least 102% of the length of the working segment 380. According to some such embodiments, the length of the support segment 390 of the endovascular device is at least 102.1% of the length of the working segment 380. According to some such embodiments, the length of the support segment 390 of the endovascular device is at least 102.2% of the length of the working segment 380. According to some such embodiments, the length of the support segment 390 of the endovascular device is at least 102.3% of the length of the working segment 380. According to some such embodiments, the length of the support segment 390 of the endovascular device is at least 102.4% of the length of the working segment 380. According to some such embodiments, the length of the support segment 390 of the endovascular device is at least 102.5% of the length of the working segment 380. According to some such embodiments, the length of the support segment 390 of the endovascular device is at least 102.6% of the length of the working segment 380. According to some such embodiments, the length of the support segment 390 of the endovascular device is at least 102.7% of the length of the working segment 380. According to some such embodiments, the length of the support segment 390 of the endovascular device is at least 102.8% of the length of the working segment 380. According to some such embodiments, the length of the support segment 390 of the endovascular device is at least 102.9% of the length of the working segment 380. According to some such embodiments, the length of the support segment 390 of the endovascular device is at least 103% of the length of the working segment 380. According to some such embodiments, the length of the support segment 390 of the endovascular device is at least 103.1% of the length of the working segment 380. According to some such embodiments, the length of the support segment 390 of the endovascular device is at least 103.2% of the length of the working segment 380. According to some such embodiments, the length of the support segment 390 of the endovascular device is at least 103.3% of the length of the working segment 380. According to some such embodiments, the length of the support segment 390 of the endovascular device is at least 103.4% of the length of the working segment 380. According to some such embodiments, the length of the support segment 390 of the endovascular device is at least 103.5% of the length of the working segment 380. According to some such embodiments, the length of the support segment 390 of the endovascular device is at least 103.6% of the length of the working segment 380. According to some such embodiments, the length of the support segment 390 of the endovascular device is at least 103.7% of the length of the working segment 380. According to some such embodiments, the length of the support segment 390 of the endovascular device is at least 103.8% of the length of the working segment 380. According to some such embodiments, the length of the support segment 390 of the endovascular device is at least 103.9% of the length of the working segment 380. According to some such embodiments, the length of the support segment 390 of the endovascular device is at least 104% of the length of the working segment 380. According to some such embodiments, the length of the support segment 390 of the endovascular device is at least 104.1% of the length of the working segment 380. According to some such embodiments, the length of the support segment 390 of the endovascular device is at least 104.2% of the length of the working segment 380. According to some such embodiments, the length of the support segment 390 of the endovascular device is at least 104.3% of the length of the working segment 380. According to some such embodiments, the length of the support segment 390 of the endovascular device is at least 104.4% of the length of the working segment 380. According to some such embodiments, the length of the support segment 390 of the endovascular device is at least 104.5% of the length of the working segment 380. According to some such embodiments, the length of the support segment 390 of the endovascular device is at least 104.6% of the length of the working segment 380. According to some such embodiments, the length of the support segment 390 of the endovascular device is at least 104.7% of the length of the working segment 380. According to some such embodiments, the length of the support segment 390 of the endovascular device is at least 104.8% of the length of the working segment 380. According to some such embodiments, the length of the support segment 390 of the endovascular device is at least 104.9% of the length of the working segment 380. According to some such embodiments, the length of the support segment 390 of the endovascular device is at least 105% of the length of the working segment 380. According to some such embodiments, the length of the support segment 390 of the endovascular device is at least 105.1% of the length of the working segment 380. According to some such embodiments, the length of the support segment 390 of the endovascular device is at least 105.2% of the length of the working segment 380. According to some such embodiments, the length of the support segment 390 of the endovascular device is at least 105.3% of the length of the working segment 380. According to some such embodiments, the length of the support segment 390 of the endovascular device is at least 105.4% of the length of the working segment 380. According to some such embodiments, the length of the support segment 390 of the endovascular device is at least 105.5% of the length of the working segment 380. According to some such embodiments, the length of the support segment 390 of the endovascular device is at least 105.6% of the length of the working segment 380. According to some such embodiments, the length of the support segment 390 of the endovascular device is at least 105.7% of the length of the working segment 380. According to some such embodiments, the length of the support segment 390 of the endovascular device is at least 105.8% of the length of the working segment 380. According to some such embodiments, the length of the support segment 390 of the endovascular device is at least 105.9% of the length of the working segment 380. According to some such embodiments, the length of the support segment 390 of the endovascular device is at least 106% of the length of the working segment 380. According to some such embodiments, the length of the support segment 390 of the endovascular device is at least 106.1% of the length of the working segment 380. According to some such embodiments, the length of the support segment 390 of the endovascular device is at least 106.2% of the length of the working segment 380. According to some such embodiments, the length of the support segment 390 of the endovascular device is at least 106.3% of the length of the working segment 380. According to some such embodiments, the length of the support segment 390 of the endovascular device is at least 106.4% of the length of the working segment 380. According to some such embodiments, the length of the support segment 390 of the endovascular device is at least 106.5% of the length of the working segment 380. According to some such embodiments, the length of the support segment 390 of the endovascular device is at least 106.6% of the length of the working segment 380. According to some such embodiments, the length of the support segment 390 of the endovascular device is at least 106.7% of the length of the working segment 380. According to some such embodiments, the length of the support segment 390 of the endovascular device is at least 106.8% of the length of the working segment 380. According to some such embodiments, the length of the support segment 390 of the endovascular device is at least 106.9% of the length of the working segment 380. According to some such embodiments, the length of the support segment 390 of the endovascular device is at least 107% of the length of the working segment 380. According to some such embodiments, the length of the support segment 390 of the endovascular device is at least 107.1% of the length of the working segment 380. According to some such embodiments, the length of the support segment 390 of the endovascular device is at least 107.2% of the length of the working segment 380. According to some such embodiments, the length of the support segment 390 of the endovascular device is at least 107.3% of the length of the working segment 380. According to some such embodiments, the length of the support segment 390 of the endovascular device is at least 107.4% of the length of the working segment 380. According to some such embodiments, the length of the support segment 390 of the endovascular device is at least 107.5% of the length of the working segment 380. According to some such embodiments, the length of the support segment 390 of the endovascular device is at least 107.6% of the length of the working segment 380. According to some such embodiments, the length of the support segment 390 of the endovascular device is at least 107.7% of the length of the working segment 380. According to some such embodiments, the length of the support segment 390 of the endovascular device is at least 107.8% of the length of the working segment 380. According to some such embodiments, the length of the support segment 390 of the endovascular device is at least 107.9% of the length of the working segment 380. According to some such embodiments, the length of the support segment 390 of the endovascular device is at least 8% of the length of the working segment 380. According to some such embodiments, the length of the support segment 390 of the endovascular device is at least 108.1% of the length of the working segment 380. According to some such embodiments, the length of the support segment 390 of the endovascular device is at least 108.2% of the length of the working segment 380. According to some such embodiments, the length of the support segment 390 of the endovascular device is at least 108.3% of the length of the working segment 380. According to some such embodiments, the length of the support segment 390 of the endovascular device is at least 108.4% of the length of the working segment 380. According to some such embodiments, the length of the support segment 390 of the endovascular device is at least 108.5% of the length of the working segment 380. According to some such embodiments, the length of the support segment 390 of the endovascular device is at least 108.6% of the length of the working segment 380. According to some such embodiments, the length of the support segment 390 of the endovascular device is at least 108.7% of the length of the working segment 380. According to some such embodiments, the length of the support segment 390 of the endovascular device is at least 108.8% of the length of the working segment 380. According to some such embodiments, the length of the support segment 390 of the endovascular device is at least 108.9% of the length of the working segment 380. According to some such embodiments, the length of the support segment 390 of the endovascular device is at least 9% of the length of the working segment 380. According to some such embodiments, the length of the support segment 390 of the endovascular device is at least 109.1% of the length of the working segment 380. According to some such embodiments, the length of the support segment 390 of the endovascular device is at least 109.2% of the length of the working segment 380. According to some such embodiments, the length of the support segment 390 of the endovascular device is at least 109.3% of the length of the working segment 380. According to some such embodiments, the length of the support segment 390 of the endovascular device is at least 109.4% of the length of the working segment 380. According to some such embodiments, the length of the support segment 390 of the endovascular device is at least 109.5% of the length of the working segment 380. According to some such embodiments, the length of the support segment 390 of the endovascular device is at least 109.6% of the length of the working segment 380. According to some such embodiments, the length of the support segment 390 of the endovascular device is at least 109.7% of the length of the working segment 380. According to some such embodiments, the length of the support segment 390 of the endovascular device is at least 109.8% of the length of the working segment 380. According to some such embodiments, the length of the support segment 390 of the endovascular device is at least 109.9% of the length of the working segment 380. According to some such embodiments, the length of the support segment 390 of the endovascular device is at least 110% of the length of the working segment 380.

According to some such embodiments, the length of the support segment 390 of the endovascular device is at least 110.1% of the length of the working segment 380. According to some such embodiments, the length of the support segment 390 of the endovascular device is at least 110.2% of the length of the working segment 380. According to some such embodiments, the length of the support segment 390 of the endovascular device is at least 110.3% of the length of the working segment 380. According to some such embodiments, the length of the support segment 390 of the endovascular device is at least 110.4% of the length of the working segment 380. According to some such embodiments, the length of the support segment 390 of the endovascular device is at least 110.5% of the length of the working segment 380. According to some such embodiments, the length of the support segment 390 of the endovascular device is at least 110.6% of the length of the working segment 380. According to some such embodiments, the length of the support segment 390 of the endovascular device is at least 110.7% of the length of the working segment 380. According to some such embodiments, the length of the support segment 390 of the endovascular device is at least 110.8% of the length of the working segment 380. According to some such embodiments, the length of the support segment 390 of the endovascular device is at least 110.9% of the length of the working segment 380. According to some such embodiments, the length of the support segment 390 of the endovascular device is at least 110% of the length of the working segment 380.

According to some such embodiments, the length of the support segment 390 of the endovascular device is at least 111.1% of the length of the working segment 380. According to some such embodiments, the length of the support segment 390 of the endovascular device is at least 111.2% of the length of the working segment 380. According to some such embodiments, the length of the support segment 390 of the endovascular device is at least 111.3% of the length of the working segment 380. According to some such embodiments, the length of the support segment 390 of the endovascular device is at least 111.4% of the length of the working segment 380. According to some such embodiments, the length of the support segment 390 of the endovascular device is at least 111.5% of the length of the working segment 380. According to some such embodiments, the length of the support segment 390 of the endovascular device is at least 111.6% of the length of the working segment 380. According to some such embodiments, the length of the support segment 390 of the endovascular device is at least 111.7% of the length of the working segment 380. According to some such embodiments, the length of the support segment 390 of the endovascular device is at least 111.8% of the length of the working segment 380. According to some such embodiments, the length of the support segment 390 of the endovascular device is at least 111.9% of the length of the working segment 380. According to some such embodiments, the length of the support segment 390 of the endovascular device is at least 112% of the length of the working segment 380.

According to some such embodiments, the length of the support segment 390 of the endovascular device is at least 112.1% of the length of the working segment 380. According to some such embodiments, the length of the support segment 390 of the endovascular device is at least 112.2% of the length of the working segment 380. According to some such embodiments, the length of the support segment 390 of the endovascular device is at least 112.3% of the length of the working segment 380. According to some such embodiments, the length of the support segment 390 of the endovascular device is at least 112.4% of the length of the working segment 380. According to some such embodiments, the length of the support segment 390 of the endovascular device is at least 112.5% of the length of the working segment 380. According to some such embodiments, the length of the support segment 390 of the endovascular device is at least 112.6% of the length of the working segment 380. According to some such embodiments, the length of the support segment 390 of the endovascular device is at least 112.7% of the length of the working segment 380. According to some such embodiments, the length of the support segment 390 of the endovascular device is at least 112.8% of the length of the working segment 380. According to some such embodiments, the length of the support segment 390 of the endovascular device is at least 112.9% of the length of the working segment 380. According to some such embodiments, the length of the support segment 390 of the endovascular device is at least 113% of the length of the working segment 380.

According to some such embodiments, the length of the support segment 390 of the endovascular device is at least 113.1% of the length of the working segment 380. According to some such embodiments, the length of the support segment 390 of the endovascular device is at least 113.2% of the length of the working segment 380. According to some such embodiments, the length of the support segment 390 of the endovascular device is at least 113.3% of the length of the working segment 380. According to some such embodiments, the length of the support segment 390 of the endovascular device is at least 113.4% of the length of the working segment 380. According to some such embodiments, the length of the support segment 390 of the endovascular device is at least 113.5% of the length of the working segment 380. According to some such embodiments, the length of the support segment 390 of the endovascular device is at least 113.6% of the length of the working segment 380. According to some such embodiments, the length of the support segment 390 of the endovascular device is at least 113.7% of the length of the working segment 380. According to some such embodiments, the length of the support segment 390 of the endovascular device is at least 113.8% of the length of the working segment 380. According to some such embodiments, the length of the support segment 390 of the endovascular device is at least 113.9% of the length of the working segment 380. According to some such embodiments, the length of the support segment 390 of the endovascular device is at least 114% of the length of the working segment 380.

According to some such embodiments, the length of the support segment 390 of the endovascular device is at least 114.1% of the length of the working segment 380. According to some such embodiments, the length of the support segment 390 of the endovascular device is at least 114.2% of the length of the working segment 380. According to some such embodiments, the length of the support segment 390 of the endovascular device is at least 114.3% of the length of the working segment 380. According to some such embodiments, the length of the support segment 390 of the endovascular device is at least 114.4% of the length of the working segment 380. According to some such embodiments, the length of the support segment 390 of the endovascular device is at least 114.5% of the length of the working segment 380. According to some such embodiments, the length of the support segment 390 of the endovascular device is at least 114.6% of the length of the working segment 380. According to some such embodiments, the length of the support segment 390 of the endovascular device is at least 114.7% of the length of the working segment 380. According to some such embodiments, the length of the support segment 390 of the endovascular device is at least 114.8% of the length of the working segment 380. According to some such embodiments, the length of the support segment 390 of the endovascular device is at least 114.9% of the length of the working segment 380. According to some such embodiments, the length of the support segment 390 of the endovascular device is at least 115% of the length of the working segment 380.

According to some such embodiments, the length of the support segment 390 of the endovascular device is at least 115.1% of the length of the working segment 380. According to some such embodiments, the length of the support segment 390 of the endovascular device is at least 115.2% of the length of the working segment 380. According to some such embodiments, the length of the support segment 390 of the endovascular device is at least 115.3% of the length of the working segment 380. According to some such embodiments, the length of the support segment 390 of the endovascular device is at least 115.4% of the length of the working segment 380. According to some such embodiments, the length of the support segment 390 of the endovascular device is at least 115.5% of the length of the working segment 380. According to some such embodiments, the length of the support segment 390 of the endovascular device is at least 115.6% of the length of the working segment 380. According to some such embodiments, the length of the support segment 390 of the endovascular device is at least 115.7% of the length of the working segment 380. According to some such embodiments, the length of the support segment 390 of the endovascular device is at least 115.8% of the length of the working segment 380. According to some such embodiments, the length of the support segment 390 of the endovascular device is at least 115.9% of the length of the working segment 380. According to some such embodiments, the length of the support segment 390 of the endovascular device is at least 116% of the length of the working segment 380.

According to some such embodiments, the length of the support segment 390 of the endovascular device is at least 116.1% of the length of the working segment 380. According to some such embodiments, the length of the support segment 390 of the endovascular device is at least 116.2% of the length of the working segment 380. According to some such embodiments, the length of the support segment 390 of the endovascular device is at least 116.3% of the length of the working segment 380. According to some such embodiments, the length of the support segment 390 of the endovascular device is at least 116.4% of the length of the working segment 380. According to some such embodiments, the length of the support segment 390 of the endovascular device is at least 116.5% of the length of the working segment 380. According to some such embodiments, the length of the support segment 390 of the endovascular device is at least 116.6% of the length of the working segment 380. According to some such embodiments, the length of the support segment 390 of the endovascular device is at least 116.7% of the length of the working segment 380. According to some such embodiments, the length of the support segment 390 of the endovascular device is at least 116.8% of the length of the working segment 380. According to some such embodiments, the length of the support segment 390 of the endovascular device is at least 116.9% of the length of the working segment 380. According to some such embodiments, the length of the support segment 390 of the endovascular device is at least 117% of the length of the working segment 380.

According to some such embodiments, the length of the support segment 390 of the endovascular device is at least 117.1% of the length of the working segment 380. According to some such embodiments, the length of the support segment 390 of the endovascular device is at least 117.2% of the length of the working segment 380. According to some such embodiments, the length of the support segment 390 of the endovascular device is at least 117.3% of the length of the working segment 380. According to some such embodiments, the length of the support segment 390 of the endovascular device is at least 117.4% of the length of the working segment 380. According to some such embodiments, the length of the support segment 390 of the endovascular device is at least 117.5% of the length of the working segment 380. According to some such embodiments, the length of the support segment 390 of the endovascular device is at least 117.6% of the length of the working segment 380. According to some such embodiments, the length of the support segment 390 of the endovascular device is at least 117.7% of the length of the working segment 380. According to some such embodiments, the length of the support segment 390 of the endovascular device is at least 117.8% of the length of the working segment 380. According to some such embodiments, the length of the support segment 390 of the endovascular device is at least 117.9% of the length of the working segment 380. According to some such embodiments, the length of the support segment 390 of the endovascular device is at least 118% of the length of the working segment 380.

According to some such embodiments, the length of the support segment 390 of the endovascular device is at least 118.1% of the length of the working segment 380. According to some such embodiments, the length of the support segment 390 of the endovascular device is at least 118.2% of the length of the working segment 380. According to some such embodiments, the length of the support segment 390 of the endovascular device is at least 118.3% of the length of the working segment 380. According to some such embodiments, the length of the support segment 390 of the endovascular device is at least 118.4% of the length of the working segment 380. According to some such embodiments, the length of the support segment 390 of the endovascular device is at least 118.5% of the length of the working segment 380. According to some such embodiments, the length of the support segment 390 of the endovascular device is at least 118.6% of the length of the working segment 380. According to some such embodiments, the length of the support segment 390 of the endovascular device is at least 118.7% of the length of the working segment 380. According to some such embodiments, the length of the support segment 390 of the endovascular device is at least 118.8% of the length of the working segment 380. According to some such embodiments, the length of the support segment 390 of the endovascular device is at least 118.9% of the length of the working segment 380. According to some such embodiments, the length of the support segment 390 of the endovascular device is at least 119% of the length of the working segment 380.

According to some such embodiments, the length of the support segment 390 of the endovascular device is at least 119.1% of the length of the working segment 380. According to some such embodiments, the length of the support segment 390 of the endovascular device is at least 119.2% of the length of the working segment 380. According to some such embodiments, the length of the support segment 390 of the endovascular device is at least 119.3% of the length of the working segment 380. According to some such embodiments, the length of the support segment 390 of the endovascular device is at least 119.4% of the length of the working segment 380. According to some such embodiments, the length of the support segment 390 of the endovascular device is at least 119.5% of the length of the working segment 380. According to some such embodiments, the length of the support segment 390 of the endovascular device is at least 119.6% of the length of the working segment 380. According to some such embodiments, the length of the support segment 390 of the endovascular device is at least 119.7% of the length of the working segment 380. According to some such embodiments, the length of the support segment 390 of the endovascular device is at least 119.8% of the length of the working segment 380. According to some such embodiments, the length of the support segment 390 of the endovascular device is at least 119.9% of the length of the working segment 380. According to some such embodiments, the length of the support segment 390 of the endovascular device is at least 120% of the length of the working segment 380.

According to some such embodiments, the length of the support segment 390 of the endovascular device is at least 120.1% of the length of the working segment 380. According to some such embodiments, the length of the support segment 390 of the endovascular device is at least 120.2% of the length of the working segment 380. According to some such embodiments, the length of the support segment 390 of the endovascular device is at least 120.3% of the length of the working segment 380. According to some such embodiments, the length of the support segment 390 of the endovascular device is at least 120.4% of the length of the working segment 380. According to some such embodiments, the length of the support segment 390 of the endovascular device is at least 120.5% of the length of the working segment 380. According to some such embodiments, the length of the support segment 390 of the endovascular device is at least 120.6% of the length of the working segment 380. According to some such embodiments, the length of the support segment 390 of the endovascular device is at least 120.7% of the length of the working segment 380. According to some such embodiments, the length of the support segment 390 of the endovascular device is at least 120.8% of the length of the working segment 380. According to some such embodiments, the length of the support segment 390 of the endovascular device is at least 120.9% of the length of the working segment 380. According to some such embodiments, the length of the support segment 390 of the endovascular device is at least 121% of the length of the working segment 380.

According to some such embodiments, the length of the support segment 390 of the endovascular device is at least 121.1% of the length of the working segment 380. According to some such embodiments, the length of the support segment 390 of the endovascular device is at least 121.2% of the length of the working segment 380. According to some such embodiments, the length of the support segment 390 of the endovascular device is at least 121.3% of the length of the working segment 380. According to some such embodiments, the length of the support segment 390 of the endovascular device is at least 121.4% of the length of the working segment 380. According to some such embodiments, the length of the support segment 390 of the endovascular device is at least 121.5% of the length of the working segment 380. According to some such embodiments, the length of the support segment 390 of the endovascular device is at least 121.6% of the length of the working segment 380. According to some such embodiments, the length of the support segment 390 of the endovascular device is at least 121.7% of the length of the working segment 380. According to some such embodiments, the length of the support segment 390 of the endovascular device is at least 121.8% of the length of the working segment 380. According to some such embodiments, the length of the support segment 390 of the endovascular device is at least 121.9% of the length of the working segment 380. According to some such embodiments, the length of the support segment 390 of the endovascular device is at least 122% of the length of the working segment 380.

According to some such embodiments, the length of the support segment 390 of the endovascular device is at least 122.1% of the length of the working segment 380. According to some such embodiments, the length of the support segment 390 of the endovascular device is at least 122.2% of the length of the working segment 380. According to some such embodiments, the length of the support segment 390 of the endovascular device is at least 122.3% of the length of the working segment 380. According to some such embodiments, the length of the support segment 390 of the endovascular device is at least 122.4% of the length of the working segment 380. According to some such embodiments, the length of the support segment 390 of the endovascular device is at least 122.5% of the length of the working segment 380. According to some such embodiments, the length of the support segment 390 of the endovascular device is at least 122.6% of the length of the working segment 380. According to some such embodiments, the length of the support segment 390 of the endovascular device is at least 122.7% of the length of the working segment 380. According to some such embodiments, the length of the support segment 390 of the endovascular device is at least 122.8% of the length of the working segment 380. According to some such embodiments, the length of the support segment 390 of the endovascular device is at least 122.9% of the length of the working segment 380. According to some such embodiments, the length of the support segment 390 of the endovascular device is at least 123% of the length of the working segment 380.

According to some such embodiments, the length of the support segment 390 of the endovascular device is at least 123.1% of the length of the working segment 380. According to some such embodiments, the length of the support segment 390 of the endovascular device is at least 123.2% of the length of the working segment 380. According to some such embodiments, the length of the support segment 390 of the endovascular device is at least 123.3% of the length of the working segment 380. According to some such embodiments, the length of the support segment 390 of the endovascular device is at least 123.4% of the length of the working segment 380. According to some such embodiments, the length of the support segment 390 of the endovascular device is at least 123.5% of the length of the working segment 380. According to some such embodiments, the length of the support segment 390 of the endovascular device is at least 123.6% of the length of the working segment 380. According to some such embodiments, the length of the support segment 390 of the endovascular device is at least 123.7% of the length of the working segment 380. According to some such embodiments, the length of the support segment 390 of the endovascular device is at least 123.8% of the length of the working segment 380. According to some such embodiments, the length of the support segment 390 of the endovascular device is at least 123.9% of the length of the working segment 380. According to some such embodiments, the length of the support segment 390 of the endovascular device is at least 124% of the length of the working segment 380.

According to some such embodiments, the length of the support segment 390 of the endovascular device is at least 124.1% of the length of the working segment 380. According to some such embodiments, the length of the support segment 390 of the endovascular device is at least 124.2% of the length of the working segment 380. According to some such embodiments, the length of the support segment 390 of the endovascular device is at least 124.3% of the length of the working segment 380. According to some such embodiments, the length of the support segment 390 of the endovascular device is at least 124.4% of the length of the working segment 380. According to some such embodiments, the length of the support segment 390 of the endovascular device is at least 124.5% of the length of the working segment 380. According to some such embodiments, the length of the support segment 390 of the endovascular device is at least 124.6% of the length of the working segment 380. According to some such embodiments, the length of the support segment 390 of the endovascular device is at least 124.7% of the length of the working segment 380. According to some such embodiments, the length of the support segment 390 of the endovascular device is at least 124.8% of the length of the working segment 380. According to some such embodiments, the length of the support segment 390 of the endovascular device is at least 124.9% of the length of the working segment 380. According to some such embodiments, the length of the support segment 390 of the endovascular device is at least 125% of the length of the working segment 380.

According to some such embodiments, the length of the support segment 390 of the endovascular device is at least 125.1% of the length of the working segment 380. According to some such embodiments, the length of the support segment 390 of the endovascular device is at least 125.2% of the length of the working segment 380. According to some such embodiments, the length of the support segment 390 of the endovascular device is at least 125.3% of the length of the working segment 380. According to some such embodiments, the length of the support segment 390 of the endovascular device is at least 125.4% of the length of the working segment 380. According to some such embodiments, the length of the support segment 390 of the endovascular device is at least 125.5% of the length of the working segment 380. According to some such embodiments, the length of the support segment 390 of the endovascular device is at least 125.6% of the length of the working segment 380. According to some such embodiments, the length of the support segment 390 of the endovascular device is at least 125.7% of the length of the working segment 380. According to some such embodiments, the length of the support segment 390 of the endovascular device is at least 125.8% of the length of the working segment 380. According to some such embodiments, the length of the support segment 390 of the endovascular device is at least 125.9% of the length of the working segment 380. According to some such embodiments, the length of the support segment 390 of the endovascular device is at least 126% of the length of the working segment 380.

According to some such embodiments, the length of the support segment 390 of the endovascular device is at least 126.1% of the length of the working segment 380. According to some such embodiments, the length of the support segment 390 of the endovascular device is at least 126.2% of the length of the working segment 380. According to some such embodiments, the length of the support segment 390 of the endovascular device is at least 126.3% of the length of the working segment 380. According to some such embodiments, the length of the support segment 390 of the endovascular device is at least 126.4% of the length of the working segment 380. According to some such embodiments, the length of the support segment 390 of the endovascular device is at least 126.5% of the length of the working segment 380. According to some such embodiments, the length of the support segment 390 of the endovascular device is at least 126.6% of the length of the working segment 380. According to some such embodiments, the length of the support segment 390 of the endovascular device is at least 126.7% of the length of the working segment 380. According to some such embodiments, the length of the support segment 390 of the endovascular device is at least 126.8% of the length of the working segment 380. According to some such embodiments, the length of the support segment 390 of the endovascular device is at least 126.9% of the length of the working segment 380. According to some such embodiments, the length of the support segment 390 of the endovascular device is at least 127% of the length of the working segment 380.

According to some such embodiments, the length of the support segment 390 of the endovascular device is at least 127.1% of the length of the working segment 380. According to some such embodiments, the length of the support segment 390 of the endovascular device is at least 127.2% of the length of the working segment 380. According to some such embodiments, the length of the support segment 390 of the endovascular device is at least 127.3% of the length of the working segment 380. According to some such embodiments, the length of the support segment 390 of the endovascular device is at least 127.4% of the length of the working segment 380. According to some such embodiments, the length of the support segment 390 of the endovascular device is at least 127.5% of the length of the working segment 380. According to some such embodiments, the length of the support segment 390 of the endovascular device is at least 127.6% of the length of the working segment 380. According to some such embodiments, the length of the support segment 390 of the endovascular device is at least 127.7% of the length of the working segment 380. According to some such embodiments, the length of the support segment 390 of the endovascular device is at least 127.8% of the length of the working segment 380. According to some such embodiments, the length of the support segment 390 of the endovascular device is at least 127.9% of the length of the working segment 380. According to some such embodiments, the length of the support segment 390 of the endovascular device is at least 128% of the length of the working segment 380.

According to some such embodiments, the length of the support segment 390 of the endovascular device is at least 128.1% of the length of the working segment 380. According to some such embodiments, the length of the support segment 390 of the endovascular device is at least 128.2% of the length of the working segment 380. According to some such embodiments, the length of the support segment 390 of the endovascular device is at least 128.3% of the length of the working segment 380. According to some such embodiments, the length of the support segment 390 of the endovascular device is at least 128.4% of the length of the working segment 380. According to some such embodiments, the length of the support segment 390 of the endovascular device is at least 128.5% of the length of the working segment 380. According to some such embodiments, the length of the support segment 390 of the endovascular device is at least 128.6% of the length of the working segment 380. According to some such embodiments, the length of the support segment 390 of the endovascular device is at least 128.7% of the length of the working segment 380. According to some such embodiments, the length of the support segment 390 of the endovascular device is at least 128.8% of the length of the working segment 380. According to some such embodiments, the length of the support segment 390 of the endovascular device is at least 128.9% of the length of the working segment 380. According to some such embodiments, the length of the support segment 390 of the endovascular device is at least 129% of the length of the working segment 380.

According to some such embodiments, the length of the support segment 390 of the endovascular device is at least 129.1% of the length of the working segment 380. According to some such embodiments, the length of the support segment 390 of the endovascular device is at least 129.2% of the length of the working segment 380. According to some such embodiments, the length of the support segment 390 of the endovascular device is at least 129.3% of the length of the working segment 380. According to some such embodiments, the length of the support segment 390 of the endovascular device is at least 129.4% of the length of the working segment 380. According to some such embodiments, the length of the support segment 390 of the endovascular device is at least 129.5% of the length of the working segment 380. According to some such embodiments, the length of the support segment 390 of the endovascular device is at least 129.6% of the length of the working segment 380. According to some such embodiments, the length of the support segment 390 of the endovascular device is at least 129.7% of the length of the working segment 380. According to some such embodiments, the length of the support segment 390 of the endovascular device is at least 129.8% of the length of the working segment 380. According to some such embodiments, the length of the support segment 390 of the endovascular device is at least 129.9% of the length of the working segment 380. According to some such embodiments, the length of the support segment 390 of the endovascular device is at least 130% of the length of the working segment 380.

According to some such embodiments, the length of the support segment 390 of the endovascular device is at least 130.1% of the length of the working segment 380. According to some such embodiments, the length of the support segment 390 of the endovascular device is at least 130.2% of the length of the working segment 380. According to some such embodiments, the length of the support segment 390 of the endovascular device is at least 130.3% of the length of the working segment 380. According to some such embodiments, the length of the support segment 390 of the endovascular device is at least 130.4% of the length of the working segment 380. According to some such embodiments, the length of the support segment 390 of the endovascular device is at least 130.5% of the length of the working segment 380. According to some such embodiments, the length of the support segment 390 of the endovascular device is at least 130.6% of the length of the working segment 380. According to some such embodiments, the length of the support segment 390 of the endovascular device is at least 130.7% of the length of the working segment 380. According to some such embodiments, the length of the support segment 390 of the endovascular device is at least 130.8% of the length of the working segment 380. According to some such embodiments, the length of the support segment 390 of the endovascular device is at least 130.9% of the length of the working segment 380. According to some such embodiments, the length of the support segment 390 of the endovascular device is at least 131% of the length of the working segment 380.

According to some such embodiments, the length of the support segment 390 of the endovascular device is at least 131.1% of the length of the working segment 380. According to some such embodiments, the length of the support segment 390 of the endovascular device is at least 131.2% of the length of the working segment 380. According to some such embodiments, the length of the support segment 390 of the endovascular device is at least 131.3% of the length of the working segment 380. According to some such embodiments, the length of the support segment 390 of the endovascular device is at least 131.4% of the length of the working segment 380. According to some such embodiments, the length of the support segment 390 of the endovascular device is at least 131.5% of the length of the working segment 380. According to some such embodiments, the length of the support segment 390 of the endovascular device is at least 131.6% of the length of the working segment 380. According to some such embodiments, the length of the support segment 390 of the endovascular device is at least 131.7% of the length of the working segment 380. According to some such embodiments, the length of the support segment 390 of the endovascular device is at least 131.8% of the length of the working segment 380. According to some such embodiments, the length of the support segment 390 of the endovascular device is at least 131.9% of the length of the working segment 380. According to some such embodiments, the length of the support segment 390 of the endovascular device is at least 132% of the length of the working segment 380.

According to some such embodiments, the length of the support segment 390 of the endovascular device is at least 132.1% of the length of the working segment 380. According to some such embodiments, the length of the support segment 390 of the endovascular device is at least 132.2% of the length of the working segment 380. According to some such embodiments, the length of the support segment 390 of the endovascular device is at least 132.3% of the length of the working segment 380. According to some such embodiments, the length of the support segment 390 of the endovascular device is at least 132.4% of the length of the working segment 380. According to some such embodiments, the length of the support segment 390 of the endovascular device is at least 132.5% of the length of the working segment 380. According to some such embodiments, the length of the support segment 390 of the endovascular device is at least 132.6% of the length of the working segment 380. According to some such embodiments, the length of the support segment 390 of the endovascular device is at least 132.7% of the length of the working segment 380. According to some such embodiments, the length of the support segment 390 of the endovascular device is at least 132.8% of the length of the working segment 380. According to some such embodiments, the length of the support segment 390 of the endovascular device is at least 132.9% of the length of the working segment 380. According to some such embodiments, the length of the support segment 390 of the endovascular device is at least 133% of the length of the working segment 380.

According to some such embodiments, the length of the support segment 390 of the endovascular device is at least 133.1% of the length of the working segment 380. According to some such embodiments, the length of the support segment 390 of the endovascular device is at least 133.2% of the length of the working segment 380. According to some such embodiments, the length of the support segment 390 of the endovascular device is at least 133.3% of the length of the working segment 380. According to some such embodiments, the length of the support segment 390 of the endovascular device is at least 133.4% of the length of the working segment 380. According to some such embodiments, the length of the support segment 390 of the endovascular device is at least 133.5% of the length of the working segment 380. According to some such embodiments, the length of the support segment 390 of the endovascular device is at least 133.6% of the length of the working segment 380. According to some such embodiments, the length of the support segment 390 of the endovascular device is at least 133.7% of the length of the working segment 380. According to some such embodiments, the length of the support segment 390 of the endovascular device is at least 133.8% of the length of the working segment 380. According to some such embodiments, the length of the support segment 390 of the endovascular device is at least 133.9% of the length of the working segment 380. According to some such embodiments, the length of the support segment 390 of the endovascular device is at least 134% of the length of the working segment 380.

According to some such embodiments, the length of the support segment 390 of the endovascular device is at least 134.1% of the length of the working segment 380. According to some such embodiments, the length of the support segment 390 of the endovascular device is at least 134.2% of the length of the working segment 380. According to some such embodiments, the length of the support segment 390 of the endovascular device is at least 134.3% of the length of the working segment 380. According to some such embodiments, the length of the support segment 390 of the endovascular device is at least 134.4% of the length of the working segment 380. According to some such embodiments, the length of the support segment 390 of the endovascular device is at least 134.5% of the length of the working segment 380. According to some such embodiments, the length of the support segment 390 of the endovascular device is at least 134.6% of the length of the working segment 380. According to some such embodiments, the length of the support segment 390 of the endovascular device is at least 134.7% of the length of the working segment 380. According to some such embodiments, the length of the support segment 390 of the endovascular device is at least 134.8% of the length of the working segment 380. According to some such embodiments, the length of the support segment 390 of the endovascular device is at least 134.9% of the length of the working segment 380. According to some such embodiments, the length of the support segment 390 of the endovascular device is at least 135% of the length of the working segment 380.

According to some such embodiments, the length of the support segment 390 of the endovascular device is at least 135.1% of the length of the working segment 380. According to some such embodiments, the length of the support segment 390 of the endovascular device is at least 135.2% of the length of the working segment 380. According to some such embodiments, the length of the support segment 390 of the endovascular device is at least 135.3% of the length of the working segment 380. According to some such embodiments, the length of the support segment 390 of the endovascular device is at least 135.4% of the length of the working segment 380. According to some such embodiments, the length of the support segment 390 of the endovascular device is at least 135.5% of the length of the working segment 380. According to some such embodiments, the length of the support segment 390 of the endovascular device is at least 135.6% of the length of the working segment 380. According to some such embodiments, the length of the support segment 390 of the endovascular device is at least 135.7% of the length of the working segment 380. According to some such embodiments, the length of the support segment 390 of the endovascular device is at least 135.8% of the length of the working segment 380. According to some such embodiments, the length of the support segment 390 of the endovascular device is at least 135.9% of the length of the working segment 380. According to some such embodiments, the length of the support segment 390 of the endovascular device is at least 136% of the length of the working segment 380.

According to some such embodiments, the length of the support segment 390 of the endovascular device is at least 136.1% of the length of the working segment 380. According to some such embodiments, the length of the support segment 390 of the endovascular device is at least 136.2% of the length of the working segment 380. According to some such embodiments, the length of the support segment 390 of the endovascular device is at least 136.3% of the length of the working segment 380. According to some such embodiments, the length of the support segment 390 of the endovascular device is at least 136.4% of the length of the working segment 380. According to some such embodiments, the length of the support segment 390 of the endovascular device is at least 136.5% of the length of the working segment 380. According to some such embodiments, the length of the support segment 390 of the endovascular device is at least 136.6% of the length of the working segment 380. According to some such embodiments, the length of the support segment 390 of the endovascular device is at least 136.7% of the length of the working segment 380. According to some such embodiments, the length of the support segment 390 of the endovascular device is at least 136.8% of the length of the working segment 380. According to some such embodiments, the length of the support segment 390 of the endovascular device is at least 136.9% of the length of the working segment 380. According to some such embodiments, the length of the support segment 390 of the endovascular device is at least 137% of the length of the working segment 380.

According to some such embodiments, the length of the support segment 390 of the endovascular device is at least 137.1% of the length of the working segment 380. According to some such embodiments, the length of the support segment 390 of the endovascular device is at least 137.2% of the length of the working segment 380. According to some such embodiments, the length of the support segment 390 of the endovascular device is at least 137.3% of the length of the working segment 380. According to some such embodiments, the length of the support segment 390 of the endovascular device is at least 137.4% of the length of the working segment 380. According to some such embodiments, the length of the support segment 390 of the endovascular device is at least 137.5% of the length of the working segment 380. According to some such embodiments, the length of the support segment 390 of the endovascular device is at least 137.6% of the length of the working segment 380. According to some such embodiments, the length of the support segment 390 of the endovascular device is at least 137.7% of the length of the working segment 380. According to some such embodiments, the length of the support segment 390 of the endovascular device is at least 137.8% of the length of the working segment 380. According to some such embodiments, the length of the support segment 390 of the endovascular device is at least 137.9% of the length of the working segment 380. According to some such embodiments, the length of the support segment 390 of the endovascular device is at least 138% of the length of the working segment 380.

According to some such embodiments, the length of the support segment 390 of the endovascular device is at least 138.1% of the length of the working segment 380. According to some such embodiments, the length of the support segment 390 of the endovascular device is at least 138.2% of the length of the working segment 380. According to some such embodiments, the length of the support segment 390 of the endovascular device is at least 138.3% of the length of the working segment 380. According to some such embodiments, the length of the support segment 390 of the endovascular device is at least 138.4% of the length of the working segment 380. According to some such embodiments, the length of the support segment 390 of the endovascular device is at least 138.5% of the length of the working segment 380. According to some such embodiments, the length of the support segment 390 of the endovascular device is at least 138.6% of the length of the working segment 380. According to some such embodiments, the length of the support segment 390 of the endovascular device is at least 138.7% of the length of the working segment 380. According to some such embodiments, the length of the support segment 390 of the endovascular device is at least 138.8% of the length of the working segment 380. According to some such embodiments, the length of the support segment 390 of the endovascular device is at least 138.9% of the length of the working segment 380. According to some such embodiments, the length of the support segment 390 of the endovascular device is at least 139% of the length of the working segment 380.

According to some such embodiments, the length of the support segment 390 of the endovascular device is at least 139.1% of the length of the working segment 380. According to some such embodiments, the length of the support segment 390 of the endovascular device is at least 139.2% of the length of the working segment 380. According to some such embodiments, the length of the support segment 390 of the endovascular device is at least 139.3% of the length of the working segment 380. According to some such embodiments, the length of the support segment 390 of the endovascular device is at least 139.4% of the length of the working segment 380. According to some such embodiments, the length of the support segment 390 of the endovascular device is at least 139.5% of the length of the working segment 380. According to some such embodiments, the length of the support segment 390 of the endovascular device is at least 139.6% of the length of the working segment 380. According to some such embodiments, the length of the support segment 390 of the endovascular device is at least 139.7% of the length of the working segment 380. According to some such embodiments, the length of the support segment 390 of the endovascular device is at least 139.8% of the length of the working segment 380. According to some such embodiments, the length of the support segment 390 of the endovascular device is at least 139.9% of the length of the working segment 380. According to some such embodiments, the length of the support segment 390 of the endovascular device is at least 140% of the length of the working segment 380.

According to some such embodiments, the length of the support segment 390 of the endovascular device is at least 140.1% of the length of the working segment 380. According to some such embodiments, the length of the support segment 390 of the endovascular device is at least 140.2% of the length of the working segment 380. According to some such embodiments, the length of the support segment 390 of the endovascular device is at least 140.3% of the length of the working segment 380. According to some such embodiments, the length of the support segment 390 of the endovascular device is at least 140.4% of the length of the working segment 380. According to some such embodiments, the length of the support segment 390 of the endovascular device is at least 140.5% of the length of the working segment 380. According to some such embodiments, the length of the support segment 390 of the endovascular device is at least 140.6% of the length of the working segment 380. According to some such embodiments, the length of the support segment 390 of the endovascular device is at least 140.7% of the length of the working segment 380. According to some such embodiments, the length of the support segment 390 of the endovascular device is at least 140.8% of the length of the working segment 380. According to some such embodiments, the length of the support segment 390 of the endovascular device is at least 140.9% of the length of the working segment 380. According to some such embodiments, the length of the support segment 390 of the endovascular device is at least 141% of the length of the working segment 380.

According to some such embodiments, the length of the support segment 390 of the endovascular device is at least 141.1% of the length of the working segment 380. According to some such embodiments, the length of the support segment 390 of the endovascular device is at least 141.2% of the length of the working segment 380. According to some such embodiments, the length of the support segment 390 of the endovascular device is at least 141.3% of the length of the working segment 380. According to some such embodiments, the length of the support segment 390 of the endovascular device is at least 141.4% of the length of the working segment 380. According to some such embodiments, the length of the support segment 390 of the endovascular device is at least 141.5% of the length of the working segment 380. According to some such embodiments, the length of the support segment 390 of the endovascular device is at least 141.6% of the length of the working segment 380. According to some such embodiments, the length of the support segment 390 of the endovascular device is at least 141.7% of the length of the working segment 380. According to some such embodiments, the length of the support segment 390 of the endovascular device is at least 141.8% of the length of the working segment 380. According to some such embodiments, the length of the support segment 390 of the endovascular device is at least 141.9% of the length of the working segment 380. According to some such embodiments, the length of the support segment 390 of the endovascular device is at least 142% of the length of the working segment 380.

According to some such embodiments, the length of the support segment 390 of the endovascular device is at least 142.1% of the length of the working segment 380. According to some such embodiments, the length of the support segment 390 of the endovascular device is at least 142.2% of the length of the working segment 380. According to some such embodiments, the length of the support segment 390 of the endovascular device is at least 142.3% of the length of the working segment 380. According to some such embodiments, the length of the support segment 390 of the endovascular device is at least 142.4% of the length of the working segment 380. According to some such embodiments, the length of the support segment 390 of the endovascular device is at least 142.5% of the length of the working segment 380. According to some such embodiments, the length of the support segment 390 of the endovascular device is at least 142.6% of the length of the working segment 380. According to some such embodiments, the length of the support segment 390 of the endovascular device is at least 142.7% of the length of the working segment 380. According to some such embodiments, the length of the support segment 390 of the endovascular device is at least 142.8% of the length of the working segment 380. According to some such embodiments, the length of the support segment 390 of the endovascular device is at least 142.9% of the length of the working segment 380. According to some such embodiments, the length of the support segment 390 of the endovascular device is at least 143% of the length of the working segment 380.

According to some such embodiments, the length of the support segment 390 of the endovascular device is at least 143.1% of the length of the working segment 380. According to some such embodiments, the length of the support segment 390 of the endovascular device is at least 143.2% of the length of the working segment 380. According to some such embodiments, the length of the support segment 390 of the endovascular device is at least 143.3% of the length of the working segment 380. According to some such embodiments, the length of the support segment 390 of the endovascular device is at least 143.4% of the length of the working segment 380. According to some such embodiments, the length of the support segment 390 of the endovascular device is at least 143.5% of the length of the working segment 380. According to some such embodiments, the length of the support segment 390 of the endovascular device is at least 143.6% of the length of the working segment 380. According to some such embodiments, the length of the support segment 390 of the endovascular device is at least 143.7% of the length of the working segment 380. According to some such embodiments, the length of the support segment 390 of the endovascular device is at least 143.8% of the length of the working segment 380. According to some such embodiments, the length of the support segment 390 of the endovascular device is at least 143.9% of the length of the working segment 380. According to some such embodiments, the length of the support segment 390 of the endovascular device is at least 144% of the length of the working segment 380.

According to some such embodiments, the length of the support segment 390 of the endovascular device is at least 144.1% of the length of the working segment 380. According to some such embodiments, the length of the support segment 390 of the endovascular device is at least 144.2% of the length of the working segment 380. According to some such embodiments, the length of the support segment 390 of the endovascular device is at least 144.3% of the length of the working segment 380. According to some such embodiments, the length of the support segment 390 of the endovascular device is at least 144.4% of the length of the working segment 380. According to some such embodiments, the length of the support segment 390 of the endovascular device is at least 144.5% of the length of the working segment 380. According to some such embodiments, the length of the support segment 390 of the endovascular device is at least 144.6% of the length of the working segment 380. According to some such embodiments, the length of the support segment 390 of the endovascular device is at least 144.7% of the length of the working segment 380. According to some such embodiments, the length of the support segment 390 of the endovascular device is at least 144.8% of the length of the working segment 380. According to some such embodiments, the length of the support segment 390 of the endovascular device is at least 144.9% of the length of the working segment 380. According to some such embodiments, the length of the support segment 390 of the endovascular device is at least 145% of the length of the working segment 380.

According to some such embodiments, the length of the support segment 390 of the endovascular device is at least 145.1% of the length of the working segment 380. According to some such embodiments, the length of the support segment 390 of the endovascular device is at least 145.2% of the length of the working segment 380. According to some such embodiments, the length of the support segment 390 of the endovascular device is at least 145.3% of the length of the working segment 380. According to some such embodiments, the length of the support segment 390 of the endovascular device is at least 145.4% of the length of the working segment 380. According to some such embodiments, the length of the support segment 390 of the endovascular device is at least 145.5% of the length of the working segment 380. According to some such embodiments, the length of the support segment 390 of the endovascular device is at least 145.6% of the length of the working segment 380. According to some such embodiments, the length of the support segment 390 of the endovascular device is at least 145.7% of the length of the working segment 380. According to some such embodiments, the length of the support segment 390 of the endovascular device is at least 145.8% of the length of the working segment 380. According to some such embodiments, the length of the support segment 390 of the endovascular device is at least 145.9% of the length of the working segment 380. According to some such embodiments, the length of the support segment 390 of the endovascular device is at least 146% of the length of the working segment 380.

According to some such embodiments, the length of the support segment 390 of the endovascular device is at least 146.1% of the length of the working segment 380. According to some such embodiments, the length of the support segment 390 of the endovascular device is at least 146.2% of the length of the working segment 380. According to some such embodiments, the length of the support segment 390 of the endovascular device is at least 146.3% of the length of the working segment 380. According to some such embodiments, the length of the support segment 390 of the endovascular device is at least 146.4% of the length of the working segment 380. According to some such embodiments, the length of the support segment 390 of the endovascular device is at least 146.5% of the length of the working segment 380. According to some such embodiments, the length of the support segment 390 of the endovascular device is at least 146.6% of the length of the working segment 380. According to some such embodiments, the length of the support segment 390 of the endovascular device is at least 146.7% of the length of the working segment 380. According to some such embodiments, the length of the support segment 390 of the endovascular device is at least 146.8% of the length of the working segment 380. According to some such embodiments, the length of the support segment 390 of the endovascular device is at least 146.9% of the length of the working segment 380. According to some such embodiments, the length of the support segment 390 of the endovascular device is at least 147% of the length of the working segment 380.

According to some such embodiments, the length of the support segment 390 of the endovascular device is at least 147.1% of the length of the working segment 380. According to some such embodiments, the length of the support segment 390 of the endovascular device is at least 147.2% of the length of the working segment 380. According to some such embodiments, the length of the support segment 390 of the endovascular device is at least 147.3% of the length of the working segment 380. According to some such embodiments, the length of the support segment 390 of the endovascular device is at least 147.4% of the length of the working segment 380. According to some such embodiments, the length of the support segment 390 of the endovascular device is at least 147.5% of the length of the working segment 380. According to some such embodiments, the length of the support segment 390 of the endovascular device is at least 147.6% of the length of the working segment 380. According to some such embodiments, the length of the support segment 390 of the endovascular device is at least 147.7% of the length of the working segment 380. According to some such embodiments, the length of the support segment 390 of the endovascular device is at least 147.8% of the length of the working segment 380. According to some such embodiments, the length of the support segment 390 of the endovascular device is at least 147.9% of the length of the working segment 380. According to some such embodiments, the length of the support segment 390 of the endovascular device is at least 148% of the length of the working segment 380.

According to some such embodiments, the length of the support segment 390 of the endovascular device is at least 148.1% of the length of the working segment 380. According to some such embodiments, the length of the support segment 390 of the endovascular device is at least 148.2% of the length of the working segment 380. According to some such embodiments, the length of the support segment 390 of the endovascular device is at least 148.3% of the length of the working segment 380. According to some such embodiments, the length of the support segment 390 of the endovascular device is at least 148.4% of the length of the working segment 380. According to some such embodiments, the length of the support segment 390 of the endovascular device is at least 148.5% of the length of the working segment 380. According to some such embodiments, the length of the support segment 390 of the endovascular device is at least 148.6% of the length of the working segment 380. According to some such embodiments, the length of the support segment 390 of the endovascular device is at least 148.7% of the length of the working segment 380. According to some such embodiments, the length of the support segment 390 of the endovascular device is at least 148.8% of the length of the working segment 380. According to some such embodiments, the length of the support segment 390 of the endovascular device is at least 148.9% of the length of the working segment 380. According to some such embodiments, the length of the support segment 390 of the endovascular device is at least 149% of the length of the working segment 380.

According to some such embodiments, the length of the support segment 390 of the endovascular device is at least 149.1% of the length of the working segment 380. According to some such embodiments, the length of the support segment 390 of the endovascular device is at least 149.2% of the length of the working segment 380. According to some such embodiments, the length of the support segment 390 of the endovascular device is at least 149.3% of the length of the working segment 380. According to some such embodiments, the length of the support segment 390 of the endovascular device is at least 149.4% of the length of the working segment 380. According to some such embodiments, the length of the support segment 390 of the endovascular device is at least 149.5% of the length of the working segment 380. According to some such embodiments, the length of the support segment 390 of the endovascular device is at least 149.6% of the length of the working segment 380. According to some such embodiments, the length of the support segment 390 of the endovascular device is at least 149.7% of the length of the working segment 380. According to some such embodiments, the length of the support segment 390 of the endovascular device is at least 149.8% of the length of the working segment 380. According to some such embodiments, the length of the support segment 390 of the endovascular device is at least 149.9% of the length of the working segment 380. According to some such embodiments, the length of the support segment 390 of the endovascular device is at least 150% of the length of the working segment 380.

According to some such embodiments, the length of the support segment 390 of the endovascular device is at least 150.1% of the length of the working segment 380. According to some such embodiments, the length of the support segment 390 of the endovascular device is at least 150.2% of the length of the working segment 380. According to some such embodiments, the length of the support segment 390 of the endovascular device is at least 150.3% of the length of the working segment 380. According to some such embodiments, the length of the support segment 390 of the endovascular device is at least 150.4% of the length of the working segment 380. According to some such embodiments, the length of the support segment 390 of the endovascular device is at least 150.5% of the length of the working segment 380. According to some such embodiments, the length of the support segment 390 of the endovascular device is at least 150.6% of the length of the working segment 380. According to some such embodiments, the length of the support segment 390 of the endovascular device is at least 150.7% of the length of the working segment 380. According to some such embodiments, the length of the support segment 390 of the endovascular device is at least 150.8% of the length of the working segment 380. According to some such embodiments, the length of the support segment 390 of the endovascular device is at least 150.9% of the length of the working segment 380. According to some such embodiments, the length of the support segment 390 of the endovascular device is at least 151% of the length of the working segment 380.

According to some such embodiments, the length of the support segment 390 of the endovascular device is at least 151.1% of the length of the working segment 380. According to some such embodiments, the length of the support segment 390 of the endovascular device is at least 151.2% of the length of the working segment 380. According to some such embodiments, the length of the support segment 390 of the endovascular device is at least 151.3% of the length of the working segment 380. According to some such embodiments, the length of the support segment 390 of the endovascular device is at least 151.4% of the length of the working segment 380. According to some such embodiments, the length of the support segment 390 of the endovascular device is at least 151.5% of the length of the working segment 380. According to some such embodiments, the length of the support segment 390 of the endovascular device is at least 151.6% of the length of the working segment 380. According to some such embodiments, the length of the support segment 390 of the endovascular device is at least 151.7% of the length of the working segment 380. According to some such embodiments, the length of the support segment 390 of the endovascular device is at least 151.8% of the length of the working segment 380. According to some such embodiments, the length of the support segment 390 of the endovascular device is at least 151.9% of the length of the working segment 380. According to some such embodiments, the length of the support segment 390 of the endovascular device is at least 152% of the length of the working segment 380.

According to some such embodiments, the length of the support segment 390 of the endovascular device is at least 152.1% of the length of the working segment 380. According to some such embodiments, the length of the support segment 390 of the endovascular device is at least 152.2% of the length of the working segment 380. According to some such embodiments, the length of the support segment 390 of the endovascular device is at least 152.3% of the length of the working segment 380. According to some such embodiments, the length of the support segment 390 of the endovascular device is at least 152.4% of the length of the working segment 380. According to some such embodiments, the length of the support segment 390 of the endovascular device is at least 152.5% of the length of the working segment 380. According to some such embodiments, the length of the support segment 390 of the endovascular device is at least 152.6% of the length of the working segment 380. According to some such embodiments, the length of the support segment 390 of the endovascular device is at least 152.7% of the length of the working segment 380. According to some such embodiments, the length of the support segment 390 of the endovascular device is at least 152.8% of the length of the working segment 380. According to some such embodiments, the length of the support segment 390 of the endovascular device is at least 152.9% of the length of the working segment 380. According to some such embodiments, the length of the support segment 390 of the endovascular device is at least 153% of the length of the working segment 380.

According to some such embodiments, the length of the support segment 390 of the endovascular device is at least 153.1% of the length of the working segment 380. According to some such embodiments, the length of the support segment 390 of the endovascular device is at least 153.2% of the length of the working segment 380. According to some such embodiments, the length of the support segment 390 of the endovascular device is at least 153.3% of the length of the working segment 380. According to some such embodiments, the length of the support segment 390 of the endovascular device is at least 153.4% of the length of the working segment 380. According to some such embodiments, the length of the support segment 390 of the endovascular device is at least 153.5% of the length of the working segment 380. According to some such embodiments, the length of the support segment 390 of the endovascular device is at least 153.6% of the length of the working segment 380. According to some such embodiments, the length of the support segment 390 of the endovascular device is at least 153.7% of the length of the working segment 380. According to some such embodiments, the length of the support segment 390 of the endovascular device is at least 153.8% of the length of the working segment 380. According to some such embodiments, the length of the support segment 390 of the endovascular device is at least 153.9% of the length of the working segment 380. According to some such embodiments, the length of the support segment 390 of the endovascular device is at least 154% of the length of the working segment 380.

According to some such embodiments, the length of the support segment 390 of the endovascular device is at least 154.1% of the length of the working segment 380. According to some such embodiments, the length of the support segment 390 of the endovascular device is at least 154.2% of the length of the working segment 380. According to some such embodiments, the length of the support segment 390 of the endovascular device is at least 154.3% of the length of the working segment 380. According to some such embodiments, the length of the support segment 390 of the endovascular device is at least 154.4% of the length of the working segment 380. According to some such embodiments, the length of the support segment 390 of the endovascular device is at least 154.5% of the length of the working segment 380. According to some such embodiments, the length of the support segment 390 of the endovascular device is at least 154.6% of the length of the working segment 380. According to some such embodiments, the length of the support segment 390 of the endovascular device is at least 154.7% of the length of the working segment 380. According to some such embodiments, the length of the support segment 390 of the endovascular device is at least 154.8% of the length of the working segment 380. According to some such embodiments, the length of the support segment 390 of the endovascular device is at least 154.9% of the length of the working segment 380. According to some such embodiments, the length of the support segment 390 of the endovascular device is at least 155% of the length of the working segment 380.

According to some such embodiments, the length of the support segment 390 of the endovascular device is at least 155.1% of the length of the working segment 380. According to some such embodiments, the length of the support segment 390 of the endovascular device is at least 155.2% of the length of the working segment 380. According to some such embodiments, the length of the support segment 390 of the endovascular device is at least 155.3% of the length of the working segment 380. According to some such embodiments, the length of the support segment 390 of the endovascular device is at least 155.4% of the length of the working segment 380. According to some such embodiments, the length of the support segment 390 of the endovascular device is at least 155.5% of the length of the working segment 380. According to some such embodiments, the length of the support segment 390 of the endovascular device is at least 155.6% of the length of the working segment 380. According to some such embodiments, the length of the support segment 390 of the endovascular device is at least 155.7% of the length of the working segment 380. According to some such embodiments, the length of the support segment 390 of the endovascular device is at least 155.8% of the length of the working segment 380. According to some such embodiments, the length of the support segment 390 of the endovascular device is at least 155.9% of the length of the working segment 380. According to some such embodiments, the length of the support segment 390 of the endovascular device is at least 156% of the length of the working segment 380.

According to some such embodiments, the length of the support segment 390 of the endovascular device is at least 156.1% of the length of the working segment 380. According to some such embodiments, the length of the support segment 390 of the endovascular device is at least 156.2% of the length of the working segment 380. According to some such embodiments, the length of the support segment 390 of the endovascular device is at least 156.3% of the length of the working segment 380. According to some such embodiments, the length of the support segment 390 of the endovascular device is at least 156.4% of the length of the working segment 380. According to some such embodiments, the length of the support segment 390 of the endovascular device is at least 156.5% of the length of the working segment 380. According to some such embodiments, the length of the support segment 390 of the endovascular device is at least 156.6% of the length of the working segment 380. According to some such embodiments, the length of the support segment 390 of the endovascular device is at least 156.7% of the length of the working segment 380. According to some such embodiments, the length of the support segment 390 of the endovascular device is at least 156.8% of the length of the working segment 380. According to some such embodiments, the length of the support segment 390 of the endovascular device is at least 156.9% of the length of the working segment 380. According to some such embodiments, the length of the support segment 390 of the endovascular device is at least 157% of the length of the working segment 380.

According to some such embodiments, the length of the support segment 390 of the endovascular device is at least 157.1% of the length of the working segment 380. According to some such embodiments, the length of the support segment 390 of the endovascular device is at least 157.2% of the length of the working segment 380. According to some such embodiments, the length of the support segment 390 of the endovascular device is at least 157.3% of the length of the working segment 380. According to some such embodiments, the length of the support segment 390 of the endovascular device is at least 157.4% of the length of the working segment 380. According to some such embodiments, the length of the support segment 390 of the endovascular device is at least 157.5% of the length of the working segment 380. According to some such embodiments, the length of the support segment 390 of the endovascular device is at least 157.6% of the length of the working segment 380. According to some such embodiments, the length of the support segment 390 of the endovascular device is at least 157.7% of the length of the working segment 380. According to some such embodiments, the length of the support segment 390 of the endovascular device is at least 157.8% of the length of the working segment 380. According to some such embodiments, the length of the support segment 390 of the endovascular device is at least 157.9% of the length of the working segment 380. According to some such embodiments, the length of the support segment 390 of the endovascular device is at least 158% of the length of the working segment 380.

According to some such embodiments, the length of the support segment 390 of the endovascular device is at least 158.1% of the length of the working segment 380. According to some such embodiments, the length of the support segment 390 of the endovascular device is at least 158.2% of the length of the working segment 380. According to some such embodiments, the length of the support segment 390 of the endovascular device is at least 158.3% of the length of the working segment 380. According to some such embodiments, the length of the support segment 390 of the endovascular device is at least 158.4% of the length of the working segment 380. According to some such embodiments, the length of the support segment 390 of the endovascular device is at least 158.5% of the length of the working segment 380. According to some such embodiments, the length of the support segment 390 of the endovascular device is at least 158.6% of the length of the working segment 380. According to some such embodiments, the length of the support segment 390 of the endovascular device is at least 158.7% of the length of the working segment 380. According to some such embodiments, the length of the support segment 390 of the endovascular device is at least 158.8% of the length of the working segment 380. According to some such embodiments, the length of the support segment 390 of the endovascular device is at least 158.9% of the length of the working segment 380. According to some such embodiments, the length of the support segment 390 of the endovascular device is at least 159% of the length of the working segment 380.

According to some such embodiments, the length of the support segment 390 of the endovascular device is at least 159.1% of the length of the working segment 380. According to some such embodiments, the length of the support segment 390 of the endovascular device is at least 159.2% of the length of the working segment 380. According to some such embodiments, the length of the support segment 390 of the endovascular device is at least 159.3% of the length of the working segment 380. According to some such embodiments, the length of the support segment 390 of the endovascular device is at least 159.4% of the length of the working segment 380. According to some such embodiments, the length of the support segment 390 of the endovascular device is at least 159.5% of the length of the working segment 380. According to some such embodiments, the length of the support segment 390 of the endovascular device is at least 159.6% of the length of the working segment 380. According to some such embodiments, the length of the support segment 390 of the endovascular device is at least 159.7% of the length of the working segment 380. According to some such embodiments, the length of the support segment 390 of the endovascular device is at least 159.8% of the length of the working segment 380. According to some such embodiments, the length of the support segment 390 of the endovascular device is at least 159.9% of the length of the working segment 380. According to some such embodiments, the length of the support segment 390 of the endovascular device is at least 160% of the length of the working segment 380.

According to some such embodiments, the length of the support segment 390 of the endovascular device is at least 160.1% of the length of the working segment 380. According to some such embodiments, the length of the support segment 390 of the endovascular device is at least 160.2% of the length of the working segment 380. According to some such embodiments, the length of the support segment 390 of the endovascular device is at least 160.3% of the length of the working segment 380. According to some such embodiments, the length of the support segment 390 of the endovascular device is at least 160.4% of the length of the working segment 380. According to some such embodiments, the length of the support segment 390 of the endovascular device is at least 160.5% of the length of the working segment 380. According to some such embodiments, the length of the support segment 390 of the endovascular device is at least 160.6% of the length of the working segment 380. According to some such embodiments, the length of the support segment 390 of the endovascular device is at least 160.7% of the length of the working segment 380. According to some such embodiments, the length of the support segment 390 of the endovascular device is at least 160.8% of the length of the working segment 380. According to some such embodiments, the length of the support segment 390 of the endovascular device is at least 160.9% of the length of the working segment 380. According to some such embodiments, the length of the support segment 390 of the endovascular device is at least 161% of the length of the working segment 380.

According to some such embodiments, the length of the support segment 390 of the endovascular device is at least 161.1% of the length of the working segment 380. According to some such embodiments, the length of the support segment 390 of the endovascular device is at least 161.2% of the length of the working segment 380. According to some such embodiments, the length of the support segment 390 of the endovascular device is at least 161.3% of the length of the working segment 380. According to some such embodiments, the length of the support segment 390 of the endovascular device is at least 161.4% of the length of the working segment 380. According to some such embodiments, the length of the support segment 390 of the endovascular device is at least 161.5% of the length of the working segment 380. According to some such embodiments, the length of the support segment 390 of the endovascular device is at least 161.6% of the length of the working segment 380. According to some such embodiments, the length of the support segment 390 of the endovascular device is at least 161.7% of the length of the working segment 380. According to some such embodiments, the length of the support segment 390 of the endovascular device is at least 161.8% of the length of the working segment 380. According to some such embodiments, the length of the support segment 390 of the endovascular device is at least 161.9% of the length of the working segment 380. According to some such embodiments, the length of the support segment 390 of the endovascular device is at least 162% of the length of the working segment 380.

According to some such embodiments, the length of the support segment 390 of the endovascular device is at least 162.1% of the length of the working segment 380. According to some such embodiments, the length of the support segment 390 of the endovascular device is at least 162.2% of the length of the working segment 380. According to some such embodiments, the length of the support segment 390 of the endovascular device is at least 162.3% of the length of the working segment 380. According to some such embodiments, the length of the support segment 390 of the endovascular device is at least 162.4% of the length of the working segment 380. According to some such embodiments, the length of the support segment 390 of the endovascular device is at least 162.5% of the length of the working segment 380. According to some such embodiments, the length of the support segment 390 of the endovascular device is at least 162.6% of the length of the working segment 380. According to some such embodiments, the length of the support segment 390 of the endovascular device is at least 162.7% of the length of the working segment 380. According to some such embodiments, the length of the support segment 390 of the endovascular device is at least 162.8% of the length of the working segment 380. According to some such embodiments, the length of the support segment 390 of the endovascular device is at least 162.9% of the length of the working segment 380. According to some such embodiments, the length of the support segment 390 of the endovascular device is at least 163% of the length of the working segment 380.

According to some such embodiments, the length of the support segment 390 of the endovascular device is at least 163.1% of the length of the working segment 380. According to some such embodiments, the length of the support segment 390 of the endovascular device is at least 163.2% of the length of the working segment 380. According to some such embodiments, the length of the support segment 390 of the endovascular device is at least 163.3% of the length of the working segment 380. According to some such embodiments, the length of the support segment 390 of the endovascular device is at least 163.4% of the length of the working segment 380. According to some such embodiments, the length of the support segment 390 of the endovascular device is at least 163.5% of the length of the working segment 380. According to some such embodiments, the length of the support segment 390 of the endovascular device is at least 163.6% of the length of the working segment 380. According to some such embodiments, the length of the support segment 390 of the endovascular device is at least 163.7% of the length of the working segment 380. According to some such embodiments, the length of the support segment 390 of the endovascular device is at least 163.8% of the length of the working segment 380. According to some such embodiments, the length of the support segment 390 of the endovascular device is at least 163.9% of the length of the working segment 380. According to some such embodiments, the length of the support segment 390 of the endovascular device is at least 164% of the length of the working segment 380.

According to some such embodiments, the length of the support segment 390 of the endovascular device is at least 164.1% of the length of the working segment 380. According to some such embodiments, the length of the support segment 390 of the endovascular device is at least 164.2% of the length of the working segment 380. According to some such embodiments, the length of the support segment 390 of the endovascular device is at least 164.3% of the length of the working segment 380. According to some such embodiments, the length of the support segment 390 of the endovascular device is at least 164.4% of the length of the working segment 380. According to some such embodiments, the length of the support segment 390 of the endovascular device is at least 164.5% of the length of the working segment 380. According to some such embodiments, the length of the support segment 390 of the endovascular device is at least 164.6% of the length of the working segment 380. According to some such embodiments, the length of the support segment 390 of the endovascular device is at least 164.7% of the length of the working segment 380. According to some such embodiments, the length of the support segment 390 of the endovascular device is at least 164.8% of the length of the working segment 380. According to some such embodiments, the length of the support segment 390 of the endovascular device is at least 164.9% of the length of the working segment 380. According to some such embodiments, the length of the support segment 390 of the endovascular device is at least 165% of the length of the working segment 380.

According to some such embodiments, the length of the support segment 390 of the endovascular device is at least 165.1% of the length of the working segment 380. According to some such embodiments, the length of the support segment 390 of the endovascular device is at least 165.2% of the length of the working segment 380. According to some such embodiments, the length of the support segment 390 of the endovascular device is at least 165.3% of the length of the working segment 380. According to some such embodiments, the length of the support segment 390 of the endovascular device is at least 165.4% of the length of the working segment 380. According to some such embodiments, the length of the support segment 390 of the endovascular device is at least 165.5% of the length of the working segment 380. According to some such embodiments, the length of the support segment 390 of the endovascular device is at least 165.6% of the length of the working segment 380. According to some such embodiments, the length of the support segment 390 of the endovascular device is at least 165.7% of the length of the working segment 380. According to some such embodiments, the length of the support segment 390 of the endovascular device is at least 165.8% of the length of the working segment 380. According to some such embodiments, the length of the support segment 390 of the endovascular device is at least 165.9% of the length of the working segment 380. According to some such embodiments, the length of the support segment 390 of the endovascular device is at least 166% of the length of the working segment 380.

According to some such embodiments, the length of the support segment 390 of the endovascular device is at least 166.1% of the length of the working segment 380. According to some such embodiments, the length of the support segment 390 of the endovascular device is at least 166.2% of the length of the working segment 380. According to some such embodiments, the length of the support segment 390 of the endovascular device is at least 166.3% of the length of the working segment 380. According to some such embodiments, the length of the support segment 390 of the endovascular device is at least 166.4% of the length of the working segment 380. According to some such embodiments, the length of the support segment 390 of the endovascular device is at least 166.5% of the length of the working segment 380. According to some such embodiments, the length of the support segment 390 of the endovascular device is at least 166.6% of the length of the working segment 380. According to some such embodiments, the length of the support segment 390 of the endovascular device is at least 166.7% of the length of the working segment 380. According to some such embodiments, the length of the support segment 390 of the endovascular device is at least 166.8% of the length of the working segment 380. According to some such embodiments, the length of the support segment 390 of the endovascular device is at least 166.9% of the length of the working segment 380. According to some such embodiments, the length of the support segment 390 of the endovascular device is at least 167% of the length of the working segment 380.

According to some such embodiments, the length of the support segment 390 of the endovascular device is at least 167.1% of the length of the working segment 380. According to some such embodiments, the length of the support segment 390 of the endovascular device is at least 167.2% of the length of the working segment 380. According to some such embodiments, the length of the support segment 390 of the endovascular device is at least 167.3% of the length of the working segment 380. According to some such embodiments, the length of the support segment 390 of the endovascular device is at least 167.4% of the length of the working segment 380. According to some such embodiments, the length of the support segment 390 of the endovascular device is at least 167.5% of the length of the working segment 380. According to some such embodiments, the length of the support segment 390 of the endovascular device is at least 167.6% of the length of the working segment 380. According to some such embodiments, the length of the support segment 390 of the endovascular device is at least 167.7% of the length of the working segment 380. According to some such embodiments, the length of the support segment 390 of the endovascular device is at least 167.8% of the length of the working segment 380. According to some such embodiments, the length of the support segment 390 of the endovascular device is at least 167.9% of the length of the working segment 380. According to some such embodiments, the length of the support segment 390 of the endovascular device is at least 168% of the length of the working segment 380.

According to some such embodiments, the length of the support segment 390 of the endovascular device is at least 168.1% of the length of the working segment 380. According to some such embodiments, the length of the support segment 390 of the endovascular device is at least 168.2% of the length of the working segment 380. According to some such embodiments, the length of the support segment 390 of the endovascular device is at least 168.3% of the length of the working segment 380. According to some such embodiments, the length of the support segment 390 of the endovascular device is at least 168.4% of the length of the working segment 380. According to some such embodiments, the length of the support segment 390 of the endovascular device is at least 168.5% of the length of the working segment 380. According to some such embodiments, the length of the support segment 390 of the endovascular device is at least 168.6% of the length of the working segment 380. According to some such embodiments, the length of the support segment 390 of the endovascular device is at least 168.7% of the length of the working segment 380. According to some such embodiments, the length of the support segment 390 of the endovascular device is at least 168.8% of the length of the working segment 380. According to some such embodiments, the length of the support segment 390 of the endovascular device is at least 168.9% of the length of the working segment 380. According to some such embodiments, the length of the support segment 390 of the endovascular device is at least 169% of the length of the working segment 380.

According to some such embodiments, the length of the support segment 390 of the endovascular device is at least 169.1% of the length of the working segment 380. According to some such embodiments, the length of the support segment 390 of the endovascular device is at least 169.2% of the length of the working segment 380. According to some such embodiments, the length of the support segment 390 of the endovascular device is at least 169.3% of the length of the working segment 380. According to some such embodiments, the length of the support segment 390 of the endovascular device is at least 169.4% of the length of the working segment 380. According to some such embodiments, the length of the support segment 390 of the endovascular device is at least 169.5% of the length of the working segment 380. According to some such embodiments, the length of the support segment 390 of the endovascular device is at least 169.6% of the length of the working segment 380. According to some such embodiments, the length of the support segment 390 of the endovascular device is at least 169.7% of the length of the working segment 380. According to some such embodiments, the length of the support segment 390 of the endovascular device is at least 169.8% of the length of the working segment 380. According to some such embodiments, the length of the support segment 390 of the endovascular device is at least 169.9% of the length of the working segment 380. According to some such embodiments, the length of the support segment 390 of the endovascular device is at least 170% of the length of the working segment 380.

According to some such embodiments, the length of the support segment 390 of the endovascular device is at least 170.1% of the length of the working segment 380. According to some such embodiments, the length of the support segment 390 of the endovascular device is at least 170.2% of the length of the working segment 380. According to some such embodiments, the length of the support segment 390 of the endovascular device is at least 170.3% of the length of the working segment 380. According to some such embodiments, the length of the support segment 390 of the endovascular device is at least 170.4% of the length of the working segment 380. According to some such embodiments, the length of the support segment 390 of the endovascular device is at least 170.5% of the length of the working segment 380. According to some such embodiments, the length of the support segment 390 of the endovascular device is at least 170.6% of the length of the working segment 380. According to some such embodiments, the length of the support segment 390 of the endovascular device is at least 170.7% of the length of the working segment 380. According to some such embodiments, the length of the support segment 390 of the endovascular device is at least 170.8% of the length of the working segment 380. According to some such embodiments, the length of the support segment 390 of the endovascular device is at least 170.9% of the length of the working segment 380. According to some such embodiments, the length of the support segment 390 of the endovascular device is at least 171% of the length of the working segment 380.

According to some such embodiments, the length of the support segment 390 of the endovascular device is at least 171.1% of the length of the working segment 380. According to some such embodiments, the length of the support segment 390 of the endovascular device is at least 171.2% of the length of the working segment 380. According to some such embodiments, the length of the support segment 390 of the endovascular device is at least 171.3% of the length of the working segment 380. According to some such embodiments, the length of the support segment 390 of the endovascular device is at least 171.4% of the length of the working segment 380. According to some such embodiments, the length of the support segment 390 of the endovascular device is at least 171.5% of the length of the working segment 380. According to some such embodiments, the length of the support segment 390 of the endovascular device is at least 171.6% of the length of the working segment 380. According to some such embodiments, the length of the support segment 390 of the endovascular device is at least 171.7% of the length of the working segment 380. According to some such embodiments, the length of the support segment 390 of the endovascular device is at least 171.8% of the length of the working segment 380. According to some such embodiments, the length of the support segment 390 of the endovascular device is at least 171.9% of the length of the working segment 380. According to some such embodiments, the length of the support segment 390 of the endovascular device is at least 172% of the length of the working segment 380.

According to some such embodiments, the length of the support segment 390 of the endovascular device is at least 172.1% of the length of the working segment 380. According to some such embodiments, the length of the support segment 390 of the endovascular device is at least 172.2% of the length of the working segment 380. According to some such embodiments, the length of the support segment 390 of the endovascular device is at least 172.3% of the length of the working segment 380. According to some such embodiments, the length of the support segment 390 of the endovascular device is at least 172.4% of the length of the working segment 380. According to some such embodiments, the length of the support segment 390 of the endovascular device is at least 172.5% of the length of the working segment 380. According to some such embodiments, the length of the support segment 390 of the endovascular device is at least 172.6% of the length of the working segment 380. According to some such embodiments, the length of the support segment 390 of the endovascular device is at least 172.7% of the length of the working segment 380. According to some such embodiments, the length of the support segment 390 of the endovascular device is at least 172.8% of the length of the working segment 380. According to some such embodiments, the length of the support segment 390 of the endovascular device is at least 172.9% of the length of the working segment 380. According to some such embodiments, the length of the support segment 390 of the endovascular device is at least 173% of the length of the working segment 380.

According to some such embodiments, the length of the support segment 390 of the endovascular device is at least 173.1% of the length of the working segment 380. According to some such embodiments, the length of the support segment 390 of the endovascular device is at least 173.2% of the length of the working segment 380. According to some such embodiments, the length of the support segment 390 of the endovascular device is at least 173.3% of the length of the working segment 380. According to some such embodiments, the length of the support segment 390 of the endovascular device is at least 173.4% of the length of the working segment 380. According to some such embodiments, the length of the support segment 390 of the endovascular device is at least 173.5% of the length of the working segment 380. According to some such embodiments, the length of the support segment 390 of the endovascular device is at least 173.6% of the length of the working segment 380. According to some such embodiments, the length of the support segment 390 of the endovascular device is at least 173.7% of the length of the working segment 380. According to some such embodiments, the length of the support segment 390 of the endovascular device is at least 173.8% of the length of the working segment 380. According to some such embodiments, the length of the support segment 390 of the endovascular device is at least 173.9% of the length of the working segment 380. According to some such embodiments, the length of the support segment 390 of the endovascular device is at least 174% of the length of the working segment 380.

According to some such embodiments, the length of the support segment 390 of the endovascular device is at least 174.1% of the length of the working segment 380. According to some such embodiments, the length of the support segment 390 of the endovascular device is at least 174.2% of the length of the working segment 380. According to some such embodiments, the length of the support segment 390 of the endovascular device is at least 174.3% of the length of the working segment 380. According to some such embodiments, the length of the support segment 390 of the endovascular device is at least 174.4% of the length of the working segment 380. According to some such embodiments, the length of the support segment 390 of the endovascular device is at least 174.5% of the length of the working segment 380. According to some such embodiments, the length of the support segment 390 of the endovascular device is at least 174.6% of the length of the working segment 380. According to some such embodiments, the length of the support segment 390 of the endovascular device is at least 174.7% of the length of the working segment 380. According to some such embodiments, the length of the support segment 390 of the endovascular device is at least 174.8% of the length of the working segment 380. According to some such embodiments, the length of the support segment 390 of the endovascular device is at least 174.9% of the length of the working segment 380. According to some such embodiments, the length of the support segment 390 of the endovascular device is at least 175% of the length of the working segment 380.

According to some such embodiments, the length of the support segment 390 of the endovascular device is at least 175.1% of the length of the working segment 380. According to some such embodiments, the length of the support segment 390 of the endovascular device is at least 175.2% of the length of the working segment 380. According to some such embodiments, the length of the support segment 390 of the endovascular device is at least 175.3% of the length of the working segment 380. According to some such embodiments, the length of the support segment 390 of the endovascular device is at least 175.4% of the length of the working segment 380. According to some such embodiments, the length of the support segment 390 of the endovascular device is at least 175.5% of the length of the working segment 380. According to some such embodiments, the length of the support segment 390 of the endovascular device is at least 175.6% of the length of the working segment 380. According to some such embodiments, the length of the support segment 390 of the endovascular device is at least 175.7% of the length of the working segment 380. According to some such embodiments, the length of the support segment 390 of the endovascular device is at least 175.8% of the length of the working segment 380. According to some such embodiments, the length of the support segment 390 of the endovascular device is at least 175.9% of the length of the working segment 380. According to some such embodiments, the length of the support segment 390 of the endovascular device is at least 176% of the length of the working segment 380.

According to some such embodiments, the length of the support segment 390 of the endovascular device is at least 176.1% of the length of the working segment 380. According to some such embodiments, the length of the support segment 390 of the endovascular device is at least 176.2% of the length of the working segment 380. According to some such embodiments, the length of the support segment 390 of the endovascular device is at least 176.3% of the length of the working segment 380. According to some such embodiments, the length of the support segment 390 of the endovascular device is at least 176.4% of the length of the working segment 380. According to some such embodiments, the length of the support segment 390 of the endovascular device is at least 176.5% of the length of the working segment 380. According to some such embodiments, the length of the support segment 390 of the endovascular device is at least 176.6% of the length of the working segment 380. According to some such embodiments, the length of the support segment 390 of the endovascular device is at least 176.7% of the length of the working segment 380. According to some such embodiments, the length of the support segment 390 of the endovascular device is at least 176.8% of the length of the working segment 380. According to some such embodiments, the length of the support segment 390 of the endovascular device is at least 176.9% of the length of the working segment 380. According to some such embodiments, the length of the support segment 390 of the endovascular device is at least 177% of the length of the working segment 380.

According to some such embodiments, the length of the support segment 390 of the endovascular device is at least 177.1% of the length of the working segment 380. According to some such embodiments, the length of the support segment 390 of the endovascular device is at least 177.2% of the length of the working segment 380. According to some such embodiments, the length of the support segment 390 of the endovascular device is at least 177.3% of the length of the working segment 380. According to some such embodiments, the length of the support segment 390 of the endovascular device is at least 177.4% of the length of the working segment 380. According to some such embodiments, the length of the support segment 390 of the endovascular device is at least 177.5% of the length of the working segment 380. According to some such embodiments, the length of the support segment 390 of the endovascular device is at least 177.6% of the length of the working segment 380. According to some such embodiments, the length of the support segment 390 of the endovascular device is at least 177.7% of the length of the working segment 380. According to some such embodiments, the length of the support segment 390 of the endovascular device is at least 177.8% of the length of the working segment 380. According to some such embodiments, the length of the support segment 390 of the endovascular device is at least 177.9% of the length of the working segment 380. According to some such embodiments, the length of the support segment 390 of the endovascular device is at least 178% of the length of the working segment 380.

According to some such embodiments, the length of the support segment 390 of the endovascular device is at least 178.1% of the length of the working segment 380. According to some such embodiments, the length of the support segment 390 of the endovascular device is at least 78.2% of the length of the working segment 380. According to some such embodiments, the length of the support segment 390 of the endovascular device is at least 178.3% of the length of the working segment 380. According to some such embodiments, the length of the support segment 390 of the endovascular device is at least 178.4% of the length of the working segment 380. According to some such embodiments, the length of the support segment 390 of the endovascular device is at least 178.5% of the length of the working segment 380. According to some such embodiments, the length of the support segment 390 of the endovascular device is at least 178.6% of the length of the working segment 380. According to some such embodiments, the length of the support segment 390 of the endovascular device is at least 178.7% of the length of the working segment 380. According to some such embodiments, the length of the support segment 390 of the endovascular device is at least 178.8% of the length of the working segment 380. According to some such embodiments, the length of the support segment 390 of the endovascular device is at least 178.9% of the length of the working segment 380. According to some such embodiments, the length of the support segment 390 of the endovascular device is at least 179% of the length of the working segment 380.

According to some such embodiments, the length of the support segment 390 of the endovascular device is at least 179.1% of the length of the working segment 380. According to some such embodiments, the length of the support segment 390 of the endovascular device is at least 179.2% of the length of the working segment 380. According to some such embodiments, the length of the support segment 390 of the endovascular device is at least 179.3% of the length of the working segment 380. According to some such embodiments, the length of the support segment 390 of the endovascular device is at least 179.4% of the length of the working segment 380. According to some such embodiments, the length of the support segment 390 of the endovascular device is at least 179.5% of the length of the working segment 380. According to some such embodiments, the length of the support segment 390 of the endovascular device is at least 179.6% of the length of the working segment 380. According to some such embodiments, the length of the support segment 390 of the endovascular device is at least 179.7% of the length of the working segment 380. According to some such embodiments, the length of the support segment 390 of the endovascular device is at least 179.8% of the length of the working segment 380. According to some such embodiments, the length of the support segment 390 of the endovascular device is at least 179.9% of the length of the working segment 380. According to some such embodiments, the length of the support segment 390 of the endovascular device is at least 180% of the length of the working segment 380.

According to some such embodiments, the length of the support segment 390 of the endovascular device is at least 180.1% of the length of the working segment 3180. According to some such embodiments, the length of the support segment 390 of the endovascular device is at least 180.2% of the length of the working segment 3180. According to some such embodiments, the length of the support segment 390 of the endovascular device is at least 180.3% of the length of the working segment 3180. According to some such embodiments, the length of the support segment 390 of the endovascular device is at least 180.4% of the length of the working segment 3180. According to some such embodiments, the length of the support segment 390 of the endovascular device is at least 180.5% of the length of the working segment 3180. According to some such embodiments, the length of the support segment 390 of the endovascular device is at least 180.6% of the length of the working segment 3180. According to some such embodiments, the length of the support segment 390 of the endovascular device is at least 180.7% of the length of the working segment 3180. According to some such embodiments, the length of the support segment 390 of the endovascular device is at least 180.8% of the length of the working segment 3180. According to some such embodiments, the length of the support segment 390 of the endovascular device is at least 180.9% of the length of the working segment 3180. According to some such embodiments, the length of the support segment 390 of the endovascular device is at least 181% of the length of the working segment 3180.

According to some such embodiments, the length of the support segment 390 of the endovascular device is at least 181.1% of the length of the working segment 380. According to some such embodiments, the length of the support segment 390 of the endovascular device is at least 181.2% of the length of the working segment 380. According to some such embodiments, the length of the support segment 390 of the endovascular device is at least 181.3% of the length of the working segment 380. According to some such embodiments, the length of the support segment 390 of the endovascular device is at least 181.4% of the length of the working segment 380. According to some such embodiments, the length of the support segment 390 of the endovascular device is at least 181.5% of the length of the working segment 380. According to some such embodiments, the length of the support segment 390 of the endovascular device is at least 181.6% of the length of the working segment 380. According to some such embodiments, the length of the support segment 390 of the endovascular device is at least 181.7% of the length of the working segment 380. According to some such embodiments, the length of the support segment 390 of the endovascular device is at least 181.8% of the length of the working segment 380. According to some such embodiments, the length of the support segment 390 of the endovascular device is at least 181.9% of the length of the working segment 380. According to some such embodiments, the length of the support segment 390 of the endovascular device is at least 182% of the length of the working segment 380.

According to some such embodiments, the length of the support segment 390 of the endovascular device is at least 182.1% of the length of the working segment 380. According to some such embodiments, the length of the support segment 390 of the endovascular device is at least 182.2% of the length of the working segment 380. According to some such embodiments, the length of the support segment 390 of the endovascular device is at least 182.3% of the length of the working segment 380. According to some such embodiments, the length of the support segment 390 of the endovascular device is at least 182.4% of the length of the working segment 380. According to some such embodiments, the length of the support segment 390 of the endovascular device is at least 182.5% of the length of the working segment 380. According to some such embodiments, the length of the support segment 390 of the endovascular device is at least 182.6% of the length of the working segment 380. According to some such embodiments, the length of the support segment 390 of the endovascular device is at least 182.7% of the length of the working segment 380. According to some such embodiments, the length of the support segment 390 of the endovascular device is at least 182.8% of the length of the working segment 380. According to some such embodiments, the length of the support segment 390 of the endovascular device is at least 182.9% of the length of the working segment 380. According to some such embodiments, the length of the support segment 390 of the endovascular device is at least 183% of the length of the working segment 380.

According to some such embodiments, the length of the support segment 390 of the endovascular device is at least 183.1% of the length of the working segment 380. According to some such embodiments, the length of the support segment 390 of the endovascular device is at least 183.2% of the length of the working segment 380. According to some such embodiments, the length of the support segment 390 of the endovascular device is at least 183.3% of the length of the working segment 380. According to some such embodiments, the length of the support segment 390 of the endovascular device is at least 183.4% of the length of the working segment 380. According to some such embodiments, the length of the support segment 390 of the endovascular device is at least 183.5% of the length of the working segment 380. According to some such embodiments, the length of the support segment 390 of the endovascular device is at least 183.6% of the length of the working segment 380. According to some such embodiments, the length of the support segment 390 of the endovascular device is at least 183.7% of the length of the working segment 380. According to some such embodiments, the length of the support segment 390 of the endovascular device is at least 183.8% of the length of the working segment 380. According to some such embodiments, the length of the support segment 390 of the endovascular device is at least 183.9% of the length of the working segment 380. According to some such embodiments, the length of the support segment 390 of the endovascular device is at least 184% of the length of the working segment 380.

According to some such embodiments, the length of the support segment 390 of the endovascular device is at least 184.1% of the length of the working segment 380. According to some such embodiments, the length of the support segment 390 of the endovascular device is at least 184.2% of the length of the working segment 380. According to some such embodiments, the length of the support segment 390 of the endovascular device is at least 184.3% of the length of the working segment 380. According to some such embodiments, the length of the support segment 390 of the endovascular device is at least 184.4% of the length of the working segment 380. According to some such embodiments, the length of the support segment 390 of the endovascular device is at least 184.5% of the length of the working segment 380. According to some such embodiments, the length of the support segment 390 of the endovascular device is at least 184.6% of the length of the working segment 380. According to some such embodiments, the length of the support segment 390 of the endovascular device is at least 184.7% of the length of the working segment 380. According to some such embodiments, the length of the support segment 390 of the endovascular device is at least 184.8% of the length of the working segment 380. According to some such embodiments, the length of the support segment 390 of the endovascular device is at least 184.9% of the length of the working segment 380. According to some such embodiments, the length of the support segment 390 of the endovascular device is at least 185% of the length of the working segment 380.

According to some such embodiments, the length of the support segment 390 of the endovascular device is at least 185.1% of the length of the working segment 380. According to some such embodiments, the length of the support segment 390 of the endovascular device is at least 185.2% of the length of the working segment 380. According to some such embodiments, the length of the support segment 390 of the endovascular device is at least 185.3% of the length of the working segment 380. According to some such embodiments, the length of the support segment 390 of the endovascular device is at least 185.4% of the length of the working segment 380. According to some such embodiments, the length of the support segment 390 of the endovascular device is at least 185.5% of the length of the working segment 380. According to some such embodiments, the length of the support segment 390 of the endovascular device is at least 185.6% of the length of the working segment 380. According to some such embodiments, the length of the support segment 390 of the endovascular device is at least 185.7% of the length of the working segment 380. According to some such embodiments, the length of the support segment 390 of the endovascular device is at least 185.8% of the length of the working segment 380. According to some such embodiments, the length of the support segment 390 of the endovascular device is at least 185.9% of the length of the working segment 380. According to some such embodiments, the length of the support segment 390 of the endovascular device is at least 186% of the length of the working segment 380.

According to some such embodiments, the length of the support segment 390 of the endovascular device is at least 186.1% of the length of the working segment 380. According to some such embodiments, the length of the support segment 390 of the endovascular device is at least 186.2% of the length of the working segment 380. According to some such embodiments, the length of the support segment 390 of the endovascular device is at least 186.3% of the length of the working segment 380. According to some such embodiments, the length of the support segment 390 of the endovascular device is at least 186.4% of the length of the working segment 380. According to some such embodiments, the length of the support segment 390 of the endovascular device is at least 186.5% of the length of the working segment 380. According to some such embodiments, the length of the support segment 390 of the endovascular device is at least 186.6% of the length of the working segment 380. According to some such embodiments, the length of the support segment 390 of the endovascular device is at least 186.7% of the length of the working segment 380. According to some such embodiments, the length of the support segment 390 of the endovascular device is at least 186.8% of the length of the working segment 380. According to some such embodiments, the length of the support segment 390 of the endovascular device is at least 186.9% of the length of the working segment 380. According to some such embodiments, the length of the support segment 390 of the endovascular device is at least 187% of the length of the working segment 380.

According to some such embodiments, the length of the support segment 390 of the endovascular device is at least 187.1% of the length of the working segment 380. According to some such embodiments, the length of the support segment 390 of the endovascular device is at least 187.2% of the length of the working segment 380. According to some such embodiments, the length of the support segment 390 of the endovascular device is at least 187.3% of the length of the working segment 380. According to some such embodiments, the length of the support segment 390 of the endovascular device is at least 187.4% of the length of the working segment 380. According to some such embodiments, the length of the support segment 390 of the endovascular device is at least 187.5% of the length of the working segment 380. According to some such embodiments, the length of the support segment 390 of the endovascular device is at least 187.6% of the length of the working segment 380. According to some such embodiments, the length of the support segment 390 of the endovascular device is at least 187.7% of the length of the working segment 380. According to some such embodiments, the length of the support segment 390 of the endovascular device is at least 187.8% of the length of the working segment 380. According to some such embodiments, the length of the support segment 390 of the endovascular device is at least 187.9% of the length of the working segment 380. According to some such embodiments, the length of the support segment 390 of the endovascular device is at least 188% of the length of the working segment 380.

According to some such embodiments, the length of the support segment 390 of the endovascular device is at least 188.1% of the length of the working segment 380. According to some such embodiments, the length of the support segment 390 of the endovascular device is at least 188.2% of the length of the working segment 380. According to some such embodiments, the length of the support segment 390 of the endovascular device is at least 188.3% of the length of the working segment 380. According to some such embodiments, the length of the support segment 390 of the endovascular device is at least 188.4% of the length of the working segment 380. According to some such embodiments, the length of the support segment 390 of the endovascular device is at least 188.5% of the length of the working segment 380. According to some such embodiments, the length of the support segment 390 of the endovascular device is at least 188.6% of the length of the working segment 380. According to some such embodiments, the length of the support segment 390 of the endovascular device is at least 188.7% of the length of the working segment 380. According to some such embodiments, the length of the support segment 390 of the endovascular device is at least 188.8% of the length of the working segment 380. According to some such embodiments, the length of the support segment 390 of the endovascular device is at least 188.9% of the length of the working segment 380. According to some such embodiments, the length of the support segment 390 of the endovascular device is at least 189% of the length of the working segment 380.

According to some such embodiments, the length of the support segment 390 of the endovascular device is at least 189.1% of the length of the working segment 380. According to some such embodiments, the length of the support segment 390 of the endovascular device is at least 189.2% of the length of the working segment 380. According to some such embodiments, the length of the support segment 390 of the endovascular device is at least 189.3% of the length of the working segment 380. According to some such embodiments, the length of the support segment 390 of the endovascular device is at least 189.4% of the length of the working segment 380. According to some such embodiments, the length of the support segment 390 of the endovascular device is at least 189.5% of the length of the working segment 380. According to some such embodiments, the length of the support segment 390 of the endovascular device is at least 189.6% of the length of the working segment 380. According to some such embodiments, the length of the support segment 390 of the endovascular device is at least 189.7% of the length of the working segment 380. According to some such embodiments, the length of the support segment 390 of the endovascular device is at least 189.8% of the length of the working segment 380. According to some such embodiments, the length of the support segment 390 of the endovascular device is at least 189.9% of the length of the working segment 380. According to some such embodiments, the length of the support segment 390 of the endovascular device is at least 190% of the length of the working segment 380.

According to some such embodiments, the length of the support segment 390 of the endovascular device is at least 190.1% of the length of the working segment 380. According to some such embodiments, the length of the support segment 390 of the endovascular device is at least 190.2% of the length of the working segment 380. According to some such embodiments, the length of the support segment 390 of the endovascular device is at least 190.3% of the length of the working segment 380. According to some such embodiments, the length of the support segment 390 of the endovascular device is at least 190.4% of the length of the working segment 380. According to some such embodiments, the length of the support segment 390 of the endovascular device is at least 190.5% of the length of the working segment 380. According to some such embodiments, the length of the support segment 390 of the endovascular device is at least 190.6% of the length of the working segment 380. According to some such embodiments, the length of the support segment 390 of the endovascular device is at least 190.7% of the length of the working segment 380. According to some such embodiments, the length of the support segment 390 of the endovascular device is at least 190.8% of the length of the working segment 380. According to some such embodiments, the length of the support segment 390 of the endovascular device is at least 190.9% of the length of the working segment 380. According to some such embodiments, the length of the support segment 390 of the endovascular device is at least 191% of the length of the working segment 380.

According to some such embodiments, the length of the support segment 390 of the endovascular device is at least 191.1% of the length of the working segment 380. According to some such embodiments, the length of the support segment 390 of the endovascular device is at least 191.2% of the length of the working segment 380. According to some such embodiments, the length of the support segment 390 of the endovascular device is at least 191.3% of the length of the working segment 380. According to some such embodiments, the length of the support segment 390 of the endovascular device is at least 91.4% of the length of the working segment 380. According to some such embodiments, the length of the support segment 390 of the endovascular device is at least 191.5% of the length of the working segment 380. According to some such embodiments, the length of the support segment 390 of the endovascular device is at least 191.6% of the length of the working segment 380. According to some such embodiments, the length of the support segment 390 of the endovascular device is at least 191.7% of the length of the working segment 380. According to some such embodiments, the length of the support segment 390 of the endovascular device is at least 191.8% of the length of the working segment 380. According to some such embodiments, the length of the support segment 390 of the endovascular device is at least 191.9% of the length of the working segment 380. According to some such embodiments, the length of the support segment 390 of the endovascular device is at least 192% of the length of the working segment 380.

According to some such embodiments, the length of the support segment 390 of the endovascular device is at least 192.1% of the length of the working segment 380. According to some such embodiments, the length of the support segment 390 of the endovascular device is at least 192.2% of the length of the working segment 380. According to some such embodiments, the length of the support segment 390 of the endovascular device is at least 192.3% of the length of the working segment 380. According to some such embodiments, the length of the support segment 390 of the endovascular device is at least 192.4% of the length of the working segment 380. According to some such embodiments, the length of the support segment 390 of the endovascular device is at least 192.5% of the length of the working segment 380. According to some such embodiments, the length of the support segment 390 of the endovascular device is at least 192.6% of the length of the working segment 380. According to some such embodiments, the length of the support segment 390 of the endovascular device is at least 192.7% of the length of the working segment 380. According to some such embodiments, the length of the support segment 390 of the endovascular device is at least 192.8% of the length of the working segment 380. According to some such embodiments, the length of the support segment 390 of the endovascular device is at least 192.9% of the length of the working segment 380. According to some such embodiments, the length of the support segment 390 of the endovascular device is at least 193% of the length of the working segment 380.

According to some such embodiments, the length of the support segment 390 of the endovascular device is at least 193.1% of the length of the working segment 380. According to some such embodiments, the length of the support segment 390 of the endovascular device is at least 193.2% of the length of the working segment 380. According to some such embodiments, the length of the support segment 390 of the endovascular device is at least 193.3% of the length of the working segment 380. According to some such embodiments, the length of the support segment 390 of the endovascular device is at least 193.4% of the length of the working segment 380. According to some such embodiments, the length of the support segment 390 of the endovascular device is at least 193.5% of the length of the working segment 380. According to some such embodiments, the length of the support segment 390 of the endovascular device is at least 193.6% of the length of the working segment 380. According to some such embodiments, the length of the support segment 390 of the endovascular device is at least 193.7% of the length of the working segment 380. According to some such embodiments, the length of the support segment 390 of the endovascular device is at least 193.8% of the length of the working segment 380. According to some such embodiments, the length of the support segment 390 of the endovascular device is at least 193.9% of the length of the working segment 380. According to some such embodiments, the length of the support segment 390 of the endovascular device is at least 194% of the length of the working segment 380.

According to some such embodiments, the length of the support segment 390 of the endovascular device is at least 194.1% of the length of the working segment 380. According to some such embodiments, the length of the support segment 390 of the endovascular device is at least 194.2% of the length of the working segment 380. According to some such embodiments, the length of the support segment 390 of the endovascular device is at least 194.3% of the length of the working segment 380. According to some such embodiments, the length of the support segment 390 of the endovascular device is at least 194.4% of the length of the working segment 380. According to some such embodiments, the length of the support segment 390 of the endovascular device is at least 194.5% of the length of the working segment 380. According to some such embodiments, the length of the support segment 390 of the endovascular device is at least 194.6% of the length of the working segment 380. According to some such embodiments, the length of the support segment 390 of the endovascular device is at least 194.7% of the length of the working segment 380. According to some such embodiments, the length of the support segment 390 of the endovascular device is at least 194.8% of the length of the working segment 380. According to some such embodiments, the length of the support segment 390 of the endovascular device is at least 194.9% of the length of the working segment 380. According to some such embodiments, the length of the support segment 390 of the endovascular device is at least 195% of the length of the working segment 380.

According to some such embodiments, the length of the support segment 390 of the endovascular device is at least 195.1% of the length of the working segment 380. According to some such embodiments, the length of the support segment 390 of the endovascular device is at least 195.2% of the length of the working segment 380. According to some such embodiments, the length of the support segment 390 of the endovascular device is at least 195.3% of the length of the working segment 380. According to some such embodiments, the length of the support segment 390 of the endovascular device is at least 195.4% of the length of the working segment 380. According to some such embodiments, the length of the support segment 390 of the endovascular device is at least 195.5% of the length of the working segment 380. According to some such embodiments, the length of the support segment 390 of the endovascular device is at least 195.6% of the length of the working segment 380. According to some such embodiments, the length of the support segment 390 of the endovascular device is at least 195.7% of the length of the working segment 380. According to some such embodiments, the length of the support segment 390 of the endovascular device is at least 195.8% of the length of the working segment 380. According to some such embodiments, the length of the support segment 390 of the endovascular device is at least 195.9% of the length of the working segment 380. According to some such embodiments, the length of the support segment 390 of the endovascular device is at least 196% of the length of the working segment 380.

According to some such embodiments, the length of the support segment 390 of the endovascular device is at least 196.1% of the length of the working segment 380. According to some such embodiments, the length of the support segment 390 of the endovascular device is at least 196.2% of the length of the working segment 380. According to some such embodiments, the length of the support segment 390 of the endovascular device is at least 196.3% of the length of the working segment 380. According to some such embodiments, the length of the support segment 390 of the endovascular device is at least 196.4% of the length of the working segment 380. According to some such embodiments, the length of the support segment 390 of the endovascular device is at least 196.5% of the length of the working segment 380. According to some such embodiments, the length of the support segment 390 of the endovascular device is at least 196.6% of the length of the working segment 380. According to some such embodiments, the length of the support segment 390 of the endovascular device is at least 196.7% of the length of the working segment 380. According to some such embodiments, the length of the support segment 390 of the endovascular device is at least 196.8% of the length of the working segment 380. According to some such embodiments, the length of the support segment 390 of the endovascular device is at least 196.9% of the length of the working segment 380. According to some such embodiments, the length of the support segment 390 of the endovascular device is at least 197% of the length of the working segment 380.

According to some such embodiments, the length of the support segment 390 of the endovascular device is at least 197.1% of the length of the working segment 380. According to some such embodiments, the length of the support segment 390 of the endovascular device is at least 197.2% of the length of the working segment 380. According to some such embodiments, the length of the support segment 390 of the endovascular device is at least 197.3% of the length of the working segment 380. According to some such embodiments, the length of the support segment 390 of the endovascular device is at least 197.4% of the length of the working segment 380. According to some such embodiments, the length of the support segment 390 of the endovascular device is at least 197.5% of the length of the working segment 380. According to some such embodiments, the length of the support segment 390 of the endovascular device is at least 197.6% of the length of the working segment 380. According to some such embodiments, the length of the support segment 390 of the endovascular device is at least 197.7% of the length of the working segment 380. According to some such embodiments, the length of the support segment 390 of the endovascular device is at least 197.8% of the length of the working segment 380. According to some such embodiments, the length of the support segment 390 of the endovascular device is at least 197.9% of the length of the working segment 380. According to some such embodiments, the length of the support segment 390 of the endovascular device is at least 198% of the length of the working segment 380.

According to some such embodiments, the length of the support segment 390 of the endovascular device is at least 198.1% of the length of the working segment 380. According to some such embodiments, the length of the support segment 390 of the endovascular device is at least 198.2% of the length of the working segment 380. According to some such embodiments, the length of the support segment 390 of the endovascular device is at least 198.3% of the length of the working segment 380. According to some such embodiments, the length of the support segment 390 of the endovascular device is at least 198.4% of the length of the working segment 380. According to some such embodiments, the length of the support segment 390 of the endovascular device is at least 198.5% of the length of the working segment 380. According to some such embodiments, the length of the support segment 390 of the endovascular device is at least 198.6% of the length of the working segment 380. According to some such embodiments, the length of the support segment 390 of the endovascular device is at least 198.7% of the length of the working segment 380. According to some such embodiments, the length of the support segment 390 of the endovascular device is at least 198.8% of the length of the working segment 380. According to some such embodiments, the length of the support segment 390 of the endovascular device is at least 198.9% of the length of the working segment 380. According to some such embodiments, the length of the support segment 390 of the endovascular device is at least 199% of the length of the working segment 380.

According to some such embodiments, the length of the support segment 390 of the endovascular device is at least 199.1% of the length of the working segment 380. According to some such embodiments, the length of the support segment 390 of the endovascular device is at least 199.2% of the length of the working segment 380. According to some such embodiments, the length of the support segment 390 of the endovascular device is at least 199.3% of the length of the working segment 380. According to some such embodiments, the length of the support segment 390 of the endovascular device is at least 199.4% of the length of the working segment 380. According to some such embodiments, the length of the support segment 390 of the endovascular device is at least 199.5% of the length of the working segment 380. According to some such embodiments, the length of the support segment 390 of the endovascular device is at least 199.6% of the length of the working segment 380. According to some such embodiments, the length of the support segment 390 of the endovascular device is at least 199.7% of the length of the working segment 380. According to some such embodiments, the length of the support segment 390 of the endovascular device is at least 199.8% of the length of the working segment 380. According to some such embodiments, the length of the support segment 390 of the endovascular device is at least 199.9% of the length of the working segment 380. According to some such embodiments, the length of the support segment 390 of the endovascular device is about 200% of the length of the working segment 380.

For example, according to some embodiments, the length of the support segment 390 of the endovascular device for intracerebral applications ranges from about 0.05 cm to about 32 cm. According to some embodiments, the length of the support segment 390 of the endovascular device ranges from about 0.05 cm to at least 32 cm. According to some embodiments, the length of the support segment 390 of the endovascular device is at least 0.05 cm. According to some embodiments, the length of the support segment 390 of the endovascular device is at least 0.1 cm. According to some embodiments, the length of the support segment 390 of the endovascular device is at least 0.2 cm. According to some embodiments, the length of the support segment 390 of the endovascular device is at least 0.3 cm. According to some embodiments, the length of the support segment 390 of the endovascular device is at least 0.4 cm. According to some embodiments, the length of the support segment 390 of the endovascular device is at least 0.5 cm. According to some embodiments, the length of the support segment 390 of the endovascular device is at least 0.6 cm. According to some embodiments, the length of the support segment 390 of the endovascular device is at least 0.7 cm. According to some embodiments, the length of the support segment 390 of the endovascular device is at least 0.8 cm. According to some embodiments, the length of the support segment 390 of the endovascular device is at least 0.9 cm. According to some embodiments, the length of the support segment 390 of the endovascular device is at least 1 cm. According to some embodiments, the length of the support segment 390 of the endovascular device is at least 1.1 cm. According to some embodiments, the length of the support segment 390 of the endovascular device is at least 1.2 cm. According to some embodiments, the length of the support segment 390 of the endovascular device is at least 1.3 cm. According to some embodiments, the length of the support segment 390 of the endovascular device is at least 1.4 cm. According to some embodiments, the length of the support segment 390 of the endovascular device is at least 1.5 cm. According to some embodiments, the length of the support segment 390 of the endovascular device is at least 1.6 cm. According to some embodiments, the length of the support segment 390 of the endovascular device is at least 1.7 cm. According to some embodiments, the length of the support segment 390 of the endovascular device is at least 1.8 cm. According to some embodiments, the length of the support segment 390 of the endovascular device is at least 1.9 cm. According to some embodiments, the length of the support segment 390 of the endovascular device is at least 2 cm. According to some embodiments, the length of the support segment 390 of the endovascular device is at least 2.1 cm. According to some embodiments, the length of the support segment 390 of the endovascular device is at least 2.2 cm. According to some embodiments, the length of the support segment 390 of the endovascular device is at least 2.3 cm. According to some embodiments, the length of the support segment 390 of the endovascular device is at least 2.4 cm. According to some embodiments, the length of the support segment 390 of the endovascular device is at least 2.5 cm. According to some embodiments, the length of the support segment 390 of the endovascular device is at least 2.6 cm. According to some embodiments, the length of the support segment 390 of the endovascular device is at least 2.7 cm. According to some embodiments, the length of the support segment 390 of the endovascular device is at least 2.8 cm. According to some embodiments, the length of the support segment 390 of the endovascular device is at least 2.9 cm. According to some embodiments, the length of the support segment 390 of the endovascular device is at least 3 cm. According to some embodiments, the length of the support segment 390 of the endovascular device is at least 3.1 cm. According to some embodiments, the length of the support segment 390 of the endovascular device is at least 3.2 cm. According to some embodiments, the length of the support segment 390 of the endovascular device is at least 3.3 cm. According to some embodiments, the length of the support segment 390 of the endovascular device is at least 3.4 cm. According to some embodiments, the length of the support segment 390 of the endovascular device is at least 3.5 cm. According to some embodiments, the length of the support segment 390 of the endovascular device is at least 3.6 cm. According to some embodiments, the length of the support segment 390 of the endovascular device is at least 3.7 cm. According to some embodiments, the length of the support segment 390 of the endovascular device is at least 3.8 cm. According to some embodiments, the length of the support segment 390 of the endovascular device is at least 3.9 cm. According to some embodiments, the length of the support segment 390 of the endovascular device is at least 4 cm. According to some embodiments, the length of the support segment 390 of the endovascular device is at least 4.1 cm. According to some embodiments, the length of the support segment 390 of the endovascular device is at least 4.2 cm. According to some embodiments, the length of the support segment 390 of the endovascular device is at least 4.3 cm. According to some embodiments, the length of the support segment 390 of the endovascular device is at least 4.4 cm. According to some embodiments, the length of the support segment 390 of the endovascular device is at least 4.5 cm. According to some embodiments, the length of the support segment 390 of the endovascular device is at least 4.6 cm. According to some embodiments, the length of the support segment 390 of the endovascular device is at least 4.7 cm. According to some embodiments, the length of the support segment 390 of the endovascular device is at least 4.8 cm. According to some embodiments, the length of the support segment 390 of the endovascular device is at least 4.9 cm. According to some embodiments, the length of the support segment 390 of the endovascular device is at least 5 cm. According to some embodiments, the length of the support segment 390 of the endovascular device is at least 5.1 cm. According to some embodiments, the length of the support segment 390 of the endovascular device is at least 5.2 cm. According to some embodiments, the length of the support segment 390 of the endovascular device is at least 5.3 cm. According to some embodiments, the length of the support segment 390 of the endovascular device is at least 5.4 cm. According to some embodiments, the length of the support segment 390 of the endovascular device is at least 5.5 cm. According to some embodiments, the length of the support segment 390 of the endovascular device is at least 5.6 cm. According to some embodiments, the length of the support segment 390 of the endovascular device is at least 5.7 cm. According to some embodiments, the length of the support segment 390 of the endovascular device is at least 5.8 cm. According to some embodiments, the length of the support segment 390 of the endovascular device is at least 5.9 cm. According to some embodiments, the length of the support segment 390 of the endovascular device is at least 6 cm. According to some embodiments, the length of the support segment 390 of the endovascular device is at least 6.1 cm. According to some embodiments, the length of the support segment 390 of the endovascular device is at least 6.2 cm. According to some embodiments, the length of the support segment 390 of the endovascular device is at least 6.3 cm. According to some embodiments, the length of the support segment 390 of the endovascular device is at least 6.4 cm. According to some embodiments, the length of the support segment 390 of the endovascular device is at least 6.5 cm. According to some embodiments, the length of the support segment 390 of the endovascular device is at least 6.6 cm. According to some embodiments, the length of the support segment 390 of the endovascular device is at least 6.7 cm. According to some embodiments, the length of the support segment 390 of the endovascular device is at least 6.8 cm. According to some embodiments, the length of the support segment 390 of the endovascular device is at least 6.9 cm. According to some embodiments, the length of the support segment 390 of the endovascular device is at least 7 cm. According to some embodiments, the length of the support segment 390 of the endovascular device is at least 7.1 cm. According to some embodiments, the length of the support segment 390 of the endovascular device is at least 7.2 cm. According to some embodiments, the length of the support segment 390 of the endovascular device is at least 7.3 cm. According to some embodiments, the length of the support segment 390 of the endovascular device is at least 7.4 cm. According to some embodiments, the length of the support segment 390 of the endovascular device is at least 7.5 cm. According to some embodiments, the length of the support segment 390 of the endovascular device is at least 7.6 cm. According to some embodiments, the length of the support segment 390 of the endovascular device is at least 7.7 cm. According to some embodiments, the length of the support segment 390 of the endovascular device is at least 7.8 cm. According to some embodiments, the length of the support segment 390 of the endovascular device is at least 7.9 cm. According to some embodiments, the length of the support segment 390 of the endovascular device is at least 8 cm. According to some embodiments, the length of the support segment 390 of the endovascular device is at least 8.1 cm. According to some embodiments, the length of the support segment 390 of the endovascular device is at least 8.2 cm. According to some embodiments, the length of the support segment 390 of the endovascular device is at least 8.3 cm. According to some embodiments, the length of the support segment 390 of the endovascular device is at least 8.4 cm. According to some embodiments, the length of the support segment 390 of the endovascular device is at least 8.5 cm. According to some embodiments, the length of the support segment 390 of the endovascular device is at least 8.6 cm. According to some embodiments, the length of the support segment 390 of the endovascular device is at least 8.7 cm. According to some embodiments, the length of the support segment 390 of the endovascular device is at least 8.8 cm. According to some embodiments, the length of the support segment 390 of the endovascular device is at least 8.9 cm. According to some embodiments, the length of the support segment 390 of the endovascular device is at least 9 cm. According to some embodiments, the length of the support segment 390 of the endovascular device is at least 9 cm. According to some embodiments, the length of the support segment 390 of the endovascular device is at least 9.1 cm. According to some embodiments, the length of the support segment 390 of the endovascular device is at least 9.2 cm. According to some embodiments, the length of the support segment 390 of the endovascular device is at least 9.3 cm. According to some embodiments, the length of the support segment 390 of the endovascular device is at least 9.4 cm. According to some embodiments, the length of the support segment 390 of the endovascular device is at least 9.5 cm. According to some embodiments, the length of the support segment 390 of the endovascular device is at least 9.6 cm. According to some embodiments, the length of the support segment 390 of the endovascular device is at least 9.7 cm. According to some embodiments, the length of the support segment 390 of the endovascular device is at least 9.8 cm. According to some embodiments, the length of the support segment 390 of the endovascular device is at least 9.9 cm. According to some embodiments, the length of the support segment 390 of the endovascular device is at least 10 cm. According to some embodiments, the length of the support segment 390 of the endovascular device is at least 10.1 cm. According to some embodiments, the length of the support segment 390 of the endovascular device is at least 10.2 cm. According to some embodiments, the length of the support segment 390 of the endovascular device is at least 10.3 cm. According to some embodiments, the length of the support segment 390 of the endovascular device is at least 10.4 cm. According to some embodiments, the length of the support segment 390 of the endovascular device is at least 10.5 cm. According to some embodiments, the length of the support segment 390 of the endovascular device is at least 10.6 cm. According to some embodiments, the length of the support segment 390 of the endovascular device is at least 10.7 cm. According to some embodiments, the length of the support segment 390 of the endovascular device is at least 10.8 cm. According to some embodiments, the length of the support segment 390 of the endovascular device is at least 10.9 cm. According to some embodiments, the length of the support segment 390 of the endovascular device is at least 11 cm. According to some embodiments, the length of the support segment 390 of the endovascular device is at least 11.1 cm. According to some embodiments, the length of the support segment 390 of the endovascular device is at least 11.2 cm. According to some embodiments, the length of the support segment 390 of the endovascular device is at least 11.3 cm. According to some embodiments, the length of the support segment 390 of the endovascular device is at least 11.4 cm. According to some embodiments, the length of the support segment 390 of the endovascular device is at least 11.5 cm. According to some embodiments, the length of the support segment 390 of the endovascular device is at least 11.6 cm. According to some embodiments, the length of the support segment 390 of the endovascular device is at least 11.7 cm. According to some embodiments, the length of the support segment 390 of the endovascular device is at least 11.8 cm. According to some embodiments, the length of the support segment 390 of the endovascular device is at least 11.9 cm. According to some embodiments, the length of the support segment 390 of the endovascular device is at least 12 cm. According to some embodiments, the length of the support segment 390 of the endovascular device is at least 12.1 cm. According to some embodiments, the length of the support segment 390 of the endovascular device is at least 12.2 cm. According to some embodiments, the length of the support segment 390 of the endovascular device is at least 12.3 cm. According to some embodiments, the length of the support segment 390 of the endovascular device is at least 12.4 cm. According to some embodiments, the length of the support segment 390 of the endovascular device is at least 12.5 cm. According to some embodiments, the length of the support segment 390 of the endovascular device is at least 12.6 cm. According to some embodiments, the length of the support segment 390 of the endovascular device is at least 12.7 cm. According to some embodiments, the length of the support segment 390 of the endovascular device is at least 12.8 cm. According to some embodiments, the length of the support segment 390 of the endovascular device is at least 12.9 cm. According to some embodiments, the length of the support segment 390 of the endovascular device is at least 13 cm. According to some embodiments, the length of the support segment 390 of the endovascular device is at least 13.1 cm. According to some embodiments, the length of the support segment 390 of the endovascular device is at least 13.2 cm. According to some embodiments, the length of the support segment 390 of the endovascular device is at least 13.3 cm. According to some embodiments, the length of the support segment 390 of the endovascular device is at least 13.4 cm. According to some embodiments, the length of the support segment 390 of the endovascular device is at least 13.5 cm. According to some embodiments, the length of the support segment 390 of the endovascular device is at least 13.6 cm. According to some embodiments, the length of the support segment 390 of the endovascular device is at least 13.7 cm. According to some embodiments, the length of the support segment 390 of the endovascular device is at least 13.8 cm. According to some embodiments, the length of the support segment 390 of the endovascular device is at least 13.9 cm. According to some embodiments, the length of the support segment 390 of the endovascular device is at least 14 cm. According to some embodiments, the length of the support segment 390 of the endovascular device is at least 14.1 cm. According to some embodiments, the length of the support segment 390 of the endovascular device is at least 14.2 cm. According to some embodiments, the length of the support segment 390 of the endovascular device is at least 14.3 cm. According to some embodiments, the length of the support segment 390 of the endovascular device is at least 14.4 cm. According to some embodiments, the length of the support segment 390 of the endovascular device is at least 14.5 cm. According to some embodiments, the length of the support segment 390 of the endovascular device is at least 14.6 cm. According to some embodiments, the length of the support segment 390 of the endovascular device is at least 14.7 cm. According to some embodiments, the length of the support segment 390 of the endovascular device is at least 14.8 cm. According to some embodiments, the length of the support segment 390 of the endovascular device is at least 14.9 cm. According to some embodiments, the length of the support segment 390 of the endovascular device is at least 15 cm. According to some embodiments, the length of the support segment 390 of the endovascular device is at least 15.1 cm. According to some embodiments, the length of the support segment 390 of the endovascular device is at least 15.2 cm. According to some embodiments, the length of the support segment 390 of the endovascular device is at least 15.3 cm. According to some embodiments, the length of the support segment 390 of the endovascular device is at least 15.4 cm. According to some embodiments, the length of the support segment 390 of the endovascular device is at least 15.5 cm. According to some embodiments, the length of the support segment 390 of the endovascular device is at least 15.6 cm. According to some embodiments, the length of the support segment 390 of the endovascular device is at least 15.7 cm. According to some embodiments, the length of the support segment 390 of the endovascular device is at least 15.8 cm. According to some embodiments, the length of the support segment 390 of the endovascular device is at least 15.9 cm. According to some embodiments, the length of the support segment 390 of the endovascular device is at least 16 cm. According to some embodiments, the length of the support segment 390 of the endovascular device is at least 16.1 cm. According to some embodiments, the length of the support segment 390 of the endovascular device is at least 16.2 cm. According to some embodiments, the length of the support segment 390 of the endovascular device is at least 16.3 cm. According to some embodiments, the length of the support segment 390 of the endovascular device is at least 16.4 cm. According to some embodiments, the length of the support segment 390 of the endovascular device is at least 16.5 cm. According to some embodiments, the length of the support segment 390 of the endovascular device is at least 16.6 cm. According to some embodiments, the length of the support segment 390 of the endovascular device is at least 16.7 cm. According to some embodiments, the length of the support segment 390 of the endovascular device is at least 16.8 cm. According to some embodiments, the length of the support segment 390 of the endovascular device is at least 16.9 cm. According to some embodiments, the length of the support segment 390 of the endovascular device is at least 17 cm. According to some embodiments, the length of the support segment 390 of the endovascular device is at least 17.1 cm. According to some embodiments, the length of the support segment 390 of the endovascular device is at least 17.2 cm. According to some embodiments, the length of the support segment 390 of the endovascular device is at least 17.3 cm. According to some embodiments, the length of the support segment 390 of the endovascular device is at least 17.4 cm. According to some embodiments, the length of the support segment 390 of the endovascular device is at least 17.5 cm. According to some embodiments, the length of the support segment 390 of the endovascular device is at least 17.6 cm. According to some embodiments, the length of the support segment 390 of the endovascular device is at least 17.7 cm. According to some embodiments, the length of the support segment 390 of the endovascular device is at least 17.8 cm. According to some embodiments, the length of the support segment 390 of the endovascular device is at least 17.9 cm. According to some embodiments, the length of the support segment 390 of the endovascular device is at least 18 cm. According to some embodiments, the length of the support segment 390 of the endovascular device is at least 18.1 cm. According to some embodiments, the length of the support segment 390 of the endovascular device is at least 18.2 cm. According to some embodiments, the length of the support segment 390 of the endovascular device is at least 18.3 cm. According to some embodiments, the length of the support segment 390 of the endovascular device is at least 18.4 cm. According to some embodiments, the length of the support segment 390 of the endovascular device is at least 18.5 cm. According to some embodiments, the length of the support segment 390 of the endovascular device is at least 18.6 cm. According to some embodiments, the length of the support segment 390 of the endovascular device is at least 18.7 cm. According to some embodiments, the length of the support segment 390 of the endovascular device is at least 18.8 cm. According to some embodiments, the length of the support segment 390 of the endovascular device is at least 18.9 cm. According to some embodiments, the length of the support segment 390 of the endovascular device is at least 19 cm. According to some embodiments, the length of the support segment 390 of the endovascular device is at least 19.1 cm. According to some embodiments, the length of the support segment 390 of the endovascular device is at least 19.2 cm. According to some embodiments, the length of the support segment 390 of the endovascular device is at least 19.3 cm. According to some embodiments, the length of the support segment 390 of the endovascular device is at least 19.4 cm. According to some embodiments, the length of the support segment 390 of the endovascular device is at least 19.5 cm. According to some embodiments, the length of the support segment 390 of the endovascular device is at least 19.6 cm. According to some embodiments, the length of the support segment 390 of the endovascular device is at least 19.7 cm. According to some embodiments, the length of the support segment 390 of the endovascular device is at least 19.8 cm. According to some embodiments, the length of the support segment 390 of the endovascular device is at least 19.9 cm. According to some embodiments, the length of the support segment 390 of the endovascular device is at least 20 cm. According to some embodiments, the length of the support segment 390 of the endovascular device is at least 20.1 cm. According to some embodiments, the length of the support segment 390 of the endovascular device is at least 20.2 cm. According to some embodiments, the length of the support segment 390 of the endovascular device is at least 20.3 cm. According to some embodiments, the length of the support segment 390 of the endovascular device is at least 20.4 cm. According to some embodiments, the length of the support segment 390 of the endovascular device is at least 20.5 cm. According to some embodiments, the length of the support segment 390 of the endovascular device is at least 20.6 cm. According to some embodiments, the length of the support segment 390 of the endovascular device is at least 20.7 cm. According to some embodiments, the length of the support segment 390 of the endovascular device is at least 20.8 cm. According to some embodiments, the length of the support segment 390 of the endovascular device is at least 20.9 cm. According to some embodiments, the length of the support segment 390 of the endovascular device is at least 21 cm. According to some embodiments, the length of the support segment 390 of the endovascular device is at least 21.1 cm. According to some embodiments, the length of the support segment 390 of the endovascular device is at least 21.2 cm. According to some embodiments, the length of the support segment 390 of the endovascular device is at least 21.3 cm. According to some embodiments, the length of the support segment 390 of the endovascular device is at least 21.4 cm. According to some embodiments, the length of the support segment 390 of the endovascular device is at least 21.5 cm. According to some embodiments, the length of the support segment 390 of the endovascular device is at least 21.6 cm. According to some embodiments, the length of the support segment 390 of the endovascular device is at least 21.7 cm. According to some embodiments, the length of the support segment 390 of the endovascular device is at least 21.8 cm. According to some embodiments, the length of the support segment 390 of the endovascular device is at least 21.9 cm. According to some embodiments, the length of the support segment 390 of the endovascular device is at least 22 cm. According to some embodiments, the length of the support segment 390 of the endovascular device is at least 22.1 cm. According to some embodiments, the length of the support segment 390 of the endovascular device is at least 22.2 cm. According to some embodiments, the length of the support segment 390 of the endovascular device is at least 22.3 cm. According to some embodiments, the length of the support segment 390 of the endovascular device is at least 22.4 cm. According to some embodiments, the length of the support segment 390 of the endovascular device is at least 22.5 cm. According to some embodiments, the length of the support segment 390 of the endovascular device is at least 22.6 cm. According to some embodiments, the length of the support segment 390 of the endovascular device is at least 22.7 cm. According to some embodiments, the length of the support segment 390 of the endovascular device is at least 22.8 cm. According to some embodiments, the length of the support segment 390 of the endovascular device is at least 22.9 cm. According to some embodiments, the length of the support segment 390 of the endovascular device is at least 23 cm. According to some embodiments, the length of the support segment 390 of the endovascular device is at least 23.1 cm. According to some embodiments, the length of the support segment 390 of the endovascular device is at least 23.2 cm. According to some embodiments, the length of the support segment 390 of the endovascular device is at least 23.3 cm. According to some embodiments, the length of the support segment 390 of the endovascular device is at least 23.4 cm. According to some embodiments, the length of the support segment 390 of the endovascular device is at least 23.5 cm. According to some embodiments, the length of the support segment 390 of the endovascular device is at least 23.6 cm. According to some embodiments, the length of the support segment 390 of the endovascular device is at least 23.7 cm. According to some embodiments, the length of the support segment 390 of the endovascular device is at least 23.8 cm. According to some embodiments, the length of the support segment 390 of the endovascular device is at least 23.9 cm. According to some embodiments, the length of the support segment 390 of the endovascular device is at least 24 cm. According to some embodiments, the length of the support segment 390 of the endovascular device is at least 24.1 cm. According to some embodiments, the length of the support segment 390 of the endovascular device is at least 24.2 cm. According to some embodiments, the length of the support segment 390 of the endovascular device is at least 24.3 cm. According to some embodiments, the length of the support segment 390 of the endovascular device is at least 24.4 cm. According to some embodiments, the length of the support segment 390 of the endovascular device is at least 24.5 cm. According to some embodiments, the length of the support segment 390 of the endovascular device is at least 24.6 cm. According to some embodiments, the length of the support segment 390 of the endovascular device is at least 24.7 cm. According to some embodiments, the length of the support segment 390 of the endovascular device is at least 24.8 cm. According to some embodiments, the length of the support segment 390 of the endovascular device is at least 24.9 cm. According to some embodiments, the length of the support segment 390 of the endovascular device is at least 25 cm. According to some embodiments, the length of the support segment 390 of the endovascular device is at least 25.1 cm. According to some embodiments, the length of the support segment 390 of the endovascular device is at least 25.2 cm. According to some embodiments, the length of the support segment 390 of the endovascular device is at least 25.3 cm. According to some embodiments, the length of the support segment 390 of the endovascular device is at least 25.4 cm. According to some embodiments, the length of the support segment 390 of the endovascular device is at least 25.5 cm. According to some embodiments, the length of the support segment 390 of the endovascular device is at least 25.6 cm. According to some embodiments, the length of the support segment 390 of the endovascular device is at least 25.7 cm. According to some embodiments, the length of the support segment 390 of the endovascular device is at least 25.8 cm. According to some embodiments, the length of the support segment 390 of the endovascular device is at least 25.9 cm. According to some embodiments, the length of the support segment 390 of the endovascular device is at least 26 cm. According to some embodiments, the length of the support segment 390 of the endovascular device is at least 26.1 cm. According to some embodiments, the length of the support segment 390 of the endovascular device is at least 26.2 cm. According to some embodiments, the length of the support segment 390 of the endovascular device is at least 26.3 cm. According to some embodiments, the length of the support segment 390 of the endovascular device is at least 26.4 cm. According to some embodiments, the length of the support segment 390 of the endovascular device is at least 26.5 cm. According to some embodiments, the length of the support segment 390 of the endovascular device is at least 26.6 cm. According to some embodiments, the length of the support segment 390 of the endovascular device is at least 26.7 cm. According to some embodiments, the length of the support segment 390 of the endovascular device is at least 26.8 cm. According to some embodiments, the length of the support segment 390 of the endovascular device is at least 26.9 cm. According to some embodiments, the length of the support segment 390 of the endovascular device is at least 27 cm. According to some embodiments, the length of the support segment 390 of the endovascular device is at least 27.1 cm. According to some embodiments, the length of the support segment 390 of the endovascular device is at least 27.2 cm. According to some embodiments, the length of the support segment 390 of the endovascular device is at least 27.3 cm. According to some embodiments, the length of the support segment 390 of the endovascular device is at least 27.4 cm. According to some embodiments, the length of the support segment 390 of the endovascular device is at least 27.5 cm. According to some embodiments, the length of the support segment 390 of the endovascular device is at least 27.6 cm. According to some embodiments, the length of the support segment 390 of the endovascular device is at least 27.7 cm. According to some embodiments, the length of the support segment 390 of the endovascular device is at least 27.8 cm. According to some embodiments, the length of the support segment 390 of the endovascular device is at least 27.9 cm. According to some embodiments, the length of the support segment 390 of the endovascular device is at least 28 cm. According to some embodiments, the length of the support segment 390 of the endovascular device is at least 28.1 cm. According to some embodiments, the length of the support segment 390 of the endovascular device is at least 28.2 cm. According to some embodiments, the length of the support segment 390 of the endovascular device is at least 28.3 cm. According to some embodiments, the length of the support segment 390 of the endovascular device is at least 28.4 cm. According to some embodiments, the length of the support segment 390 of the endovascular device is at least 28.5 cm. According to some embodiments, the length of the support segment 390 of the endovascular device is at least 28.6 cm. According to some embodiments, the length of the support segment 390 of the endovascular device is at least 28.7 cm. According to some embodiments, the length of the support segment 390 of the endovascular device is at least 28.8 cm. According to some embodiments, the length of the support segment 390 of the endovascular device is at least 28.9 cm. According to some embodiments, the length of the support segment 390 of the endovascular device is at least 29 cm. According to some embodiments, the length of the support segment 390 of the endovascular device is at least 29.1 cm. According to some embodiments, the length of the support segment 390 of the endovascular device is at least 29.2 cm. According to some embodiments, the length of the support segment 390 of the endovascular device is at least 29.3 cm. According to some embodiments, the length of the support segment 390 of the endovascular device is at least 29.4 cm. According to some embodiments, the length of the support segment 390 of the endovascular device is at least 29.5 cm. According to some embodiments, the length of the support segment 390 of the endovascular device is at least 29.6 cm. According to some embodiments, the length of the support segment 390 of the endovascular device is at least 29.7 cm. According to some embodiments, the length of the support segment 390 of the endovascular device is at least 29.8 cm. According to some embodiments, the length of the support segment 390 of the endovascular device is at least 29.9 cm. According to some embodiments, the length of the support segment 390 of the endovascular device is at least 30 cm. According to some embodiments, the length of the support segment 390 of the endovascular device is at least 30.1 cm. According to some embodiments, the length of the support segment 390 of the endovascular device is at least 30.2 cm. According to some embodiments, the length of the support segment 390 of the endovascular device is at least 30.3 cm. According to some embodiments, the length of the support segment 390 of the endovascular device is at least 30.4 cm. According to some embodiments, the length of the support segment 390 of the endovascular device is at least 30.5 cm. According to some embodiments, the length of the support segment 390 of the endovascular device is at least 30.6 cm. According to some embodiments, the length of the support segment 390 of the endovascular device is at least 30.7 cm. According to some embodiments, the length of the support segment 390 of the endovascular device is at least 30.8 cm. According to some embodiments, the length of the support segment 390 of the endovascular device is at least 30.9 cm. According to some embodiments, the length of the support segment 390 of the endovascular device is at least 31 cm. According to some embodiments, the length of the support segment 390 of the endovascular device is at least 31.1 cm. According to some embodiments, the length of the support segment 390 of the endovascular device is at least 31.2 cm. According to some embodiments, the length of the support segment 390 of the endovascular device is at least 31.3 cm. According to some embodiments, the length of the support segment 390 of the endovascular device is at least 31.4 cm. According to some embodiments, the length of the support segment 390 of the endovascular device is at least 31.5 cm. According to some embodiments, the length of the support segment 390 of the endovascular device is at least 31.6 cm. According to some embodiments, the length of the support segment 390 of the endovascular device is at least 31.7 cm. According to some embodiments, the length of the support segment 390 of the endovascular device is at least 31.8 cm. According to some embodiments, the length of the support segment 390 of the endovascular device is at least 31.9 cm. According to some embodiments, the length of the support segment 390 of the endovascular device is at least 32 cm.

For example, according to some embodiments, the length of the support segment 390 of the endovascular device for peripheral applications ranges from about 0.05 cm to about 320 cm. According to some embodiments, the length of the support segment 390 of the endovascular device ranges from about 0.05 cm to at least 320 cm.

According to some embodiments, the support segment 390 of the endovascular device comprises an inflatable balloon. According to some embodiments, the inflatable balloon is attached to the distal portion of the support segment 390 of the endovascular device. According to some embodiments, the inflatable balloon is attached proximal to the side-hole 310. According to some embodiments, the inflatable balloon is attached distal to the side-hole 310. According to some embodiments, the inflatable balloon is attached opposite the side-hole 310. According to some embodiments, the inflatable balloon is attached opposite the side-hole 310 and spans the length of the side-hole 310. According to some embodiments, the inflatable balloon is attached to a second device separate from the endovascular device 300. According to some embodiments, the second separate device is a segment of an inflatable balloon catheter. According to some embodiments, the segment of the inflatable balloon catheter comprises a luer lock on its proximal end. According to some embodiments, the second separate device can be advanced into a blood vessel through the lumen defined in the support segment 390 and/or the lumen defined in the working segment 380 of the endovascular device. According to some embodiments, the inflated balloon is effective to anchor the support segment 390 of the endovascular device to a blood vessel. According to some embodiments, the blood vessel is an artery. According to some embodiments, the blood vessel is a vein.

According to some embodiments, endovascular device 300 is the inner catheter of endovascular device 1400.

According to some embodiments, the end 350 is open. According to some embodiments, the end 350 is closed.

According to some embodiments, the second segment 330 extends from about 0.6 cm to about 200 cm in length from the side-hole 310. According to some embodiments, the second segment 330 extends from about 0.6 cm to at least 200 cm in length from the side-hole 310. According to some embodiments, the second segment 330 extends at least 0.6 cm in length from the side-hole 310. According to some embodiments, the second segment 330 extends at least 0.7 cm in length from the side-hole 310. According to some embodiments, the second segment 330 extends at least 0.8 cm in length from the side-hole 310. According to some embodiments, the second segment 330 extends at least 0.9 cm in length from the side-hole 310. According to some embodiments, the second segment 330 extends at least 1 cm in length from the side-hole 310. According to some embodiments, the second segment 330 extends at least 2 cm in length from the side-hole 310. According to some embodiments, the second segment 330 extends at least 3 cm in length from the side-hole 310. According to some embodiments, the second segment 330 extends at least 4 cm in length from the side-hole 310. According to some embodiments, the second segment 330 extends at least 5 cm in length from the side-hole 310. According to some embodiments, the second segment 330 extends at least 6 cm in length from the side-hole 310. According to some embodiments, the second segment 330 extends at least 7 cm in length from the side-hole 310. According to some embodiments, the second segment 330 extends at least 8 cm in length from the side-hole 310. According to some embodiments, the second segment 330 extends at least 9 cm in length from the side-hole 310. According to some embodiments, the second segment 330 extends at least 10 cm in length from the side-hole 310. According to some embodiments, the second segment 330 extends at least 11 cm in length from the side-hole 310. According to some embodiments, the second segment 330 extends at least 12 cm in length from the side-hole 310. According to some embodiments, the second segment 330 extends at least 13 cm in length from the side-hole 310. According to some embodiments, the second segment 330 extends at least 14 cm in length from the side-hole 310. According to some embodiments, the second segment 330 extends at least 15 cm in length from the side-hole 310. According to some embodiments, the second segment 330 extends at least 16 cm in length from the side-hole 310. According to some embodiments, the second segment 330 extends at least 17 cm in length from the side-hole 310. According to some embodiments, the second segment 330 extends at least 18 cm in length from the side-hole 310. According to some embodiments, the second segment 330 extends at least 19 cm in length from the side-hole 310. According to some embodiments, the second segment 330 extends at least 20 cm in length from the side-hole 310. According to some embodiments, the second segment 330 extends at least 21 cm in length from the side-hole 310. According to some embodiments, the second segment 330 extends at least 22 cm in length from the side-hole 310. According to some embodiments, the second segment 330 extends at least 23 cm in length from the side-hole 310. According to some embodiments, the second segment 330 extends at least 24 cm in length from the side-hole 310. According to some embodiments, the second segment 330 extends at least 25 cm in length from the side-hole 310. According to some embodiments, the second segment 330 extends at least 26 cm in length from the side-hole 310. According to some embodiments, the second segment 330 extends at least 27 cm in length from the side-hole 310. According to some embodiments, the second segment 330 extends at least 28 cm in length from the side-hole 310. According to some embodiments, the second segment 330 extends at least 29 cm in length from the side-hole 310. According to some embodiments, the second segment 330 extends at least 30 cm in length from the side-hole 310. According to some embodiments, the second segment 330 extends at least 31 cm in length from the side-hole 310. According to some embodiments, the second segment 330 extends at least 32 cm in length from the side-hole 310. According to some embodiments, the second segment 330 extends at least 33 cm in length from the side-hole 310. According to some embodiments, the second segment 330 extends at least 34 cm in length from the side-hole 310. According to some embodiments, the second segment 330 extends at least 35 cm in length from the side-hole 310. According to some embodiments, the second segment 330 extends at least 36 cm in length from the side-hole 310. According to some embodiments, the second segment 330 extends at least 37 cm in length from the side-hole 310. According to some embodiments, the second segment 330 extends at least 38 cm in length from the side-hole 310. According to some embodiments, the second segment 330 extends at least 39 cm in length from the side-hole 310. According to some embodiments, the second segment 330 extends at least 40 cm in length from the side-hole 310. According to some embodiments, the second segment 330 extends at least 41 cm in length from the side-hole 310. According to some embodiments, the second segment 330 extends at least 42 cm in length from the side-hole 310. According to some embodiments, the second segment 330 extends at least 43 cm in length from the side-hole 310. According to some embodiments, the second segment 330 extends at least 44 cm in length from the side-hole 310. According to some embodiments, the second segment 330 extends at least 45 cm in length from the side-hole 310. According to some embodiments, the second segment 330 extends at least 46 cm in length from the side-hole 310. According to some embodiments, the second segment 330 extends at least 47 cm in length from the side-hole 310. According to some embodiments, the second segment 330 extends at least 48 cm in length from the side-hole 310. According to some embodiments, the second segment 330 extends at least 49 cm in length from the side-hole 310. According to some embodiments, the second segment 330 extends at least 50 cm in length from the side-hole 310. According to some embodiments, the second segment 330 extends at least 51 cm in length from the side-hole 310. According to some embodiments, the second segment 330 extends at least 52 cm in length from the side-hole 310. According to some embodiments, the second segment 330 extends at least 53 cm in length from the side-hole 310. According to some embodiments, the second segment 330 extends at least 54 cm in length from the side-hole 310. According to some embodiments, the second segment 330 extends at least 55 cm in length from the side-hole 310. According to some embodiments, the second segment 330 extends at least 56 cm in length from the side-hole 310. According to some embodiments, the second segment 330 extends at least 57 cm in length from the side-hole 310. According to some embodiments, the second segment 330 extends at least 58 cm in length from the side-hole 310. According to some embodiments, the second segment 330 extends at least 59 cm in length from the side-hole 310. According to some embodiments, the second segment 330 extends at least 60 cm in length from the side-hole 310.

According to some embodiments, the second segment 330 extends at least 61 cm in length from the side-hole 310. According to some embodiments, the second segment 330 extends at least 62 cm in length from the side-hole 310. According to some embodiments, the second segment 330 extends at least 63 cm in length from the side-hole 310. According to some embodiments, the second segment 330 extends at least 64 cm in length from the side-hole 310. According to some embodiments, the second segment 330 extends at least 65 cm in length from the side-hole 310. According to some embodiments, the second segment 330 extends at least 66 cm in length from the side-hole 310. According to some embodiments, the second segment 330 extends at least 67 cm in length from the side-hole 310. According to some embodiments, the second segment 330 extends at least 68 cm in length from the side-hole 310. According to some embodiments, the second segment 330 extends at least 69 cm in length from the side-hole 310. According to some embodiments, the second segment 330 extends at least 70 cm in length from the side-hole 310.

According to some embodiments, the second segment 330 extends at least 71 cm in length from the side-hole 310. According to some embodiments, the second segment 330 extends at least 72 cm in length from the side-hole 310. According to some embodiments, the second segment 330 extends at least 73 cm in length from the side-hole 310. According to some embodiments, the second segment 330 extends at least 74 cm in length from the side-hole 310. According to some embodiments, the second segment 330 extends at least 75 cm in length from the side-hole 310. According to some embodiments, the second segment 330 extends at least 76 cm in length from the side-hole 310. According to some embodiments, the second segment 330 extends at least 77 cm in length from the side-hole 310. According to some embodiments, the second segment 330 extends at least 78 cm in length from the side-hole 310. According to some embodiments, the second segment 330 extends at least 79 cm in length from the side-hole 310. According to some embodiments, the second segment 330 extends at least 80 cm in length from the side-hole 310.

According to some embodiments, the second segment 330 extends at least 81 cm in length from the side-hole 310. According to some embodiments, the second segment 330 extends at least 82 cm in length from the side-hole 310. According to some embodiments, the second segment 330 extends at least 83 cm in length from the side-hole 310. According to some embodiments, the second segment 330 extends at least 84 cm in length from the side-hole 310. According to some embodiments, the second segment 330 extends at least 85 cm in length from the side-hole 310. According to some embodiments, the second segment 330 extends at least 86 cm in length from the side-hole 310. According to some embodiments, the second segment 330 extends at least 87 cm in length from the side-hole 310. According to some embodiments, the second segment 330 extends at least 88 cm in length from the side-hole 310. According to some embodiments, the second segment 330 extends at least 89 cm in length from the side-hole 310. According to some embodiments, the second segment 330 extends at least 90 cm in length from the side-hole 310.

According to some embodiments, the second segment 330 extends at least 91 cm in length from the side-hole 310. According to some embodiments, the second segment 330 extends at least 92 cm in length from the side-hole 310. According to some embodiments, the second segment 330 extends at least 93 cm in length from the side-hole 310. According to some embodiments, the second segment 330 extends at least 94 cm in length from the side-hole 310. According to some embodiments, the second segment 330 extends at least 95 cm in length from the side-hole 310. According to some embodiments, the second segment 330 extends at least 96 cm in length from the side-hole 310. According to some embodiments, the second segment 330 extends at least 97 cm in length from the side-hole 310. According to some embodiments, the second segment 330 extends at least 98 cm in length from the side-hole 310. According to some embodiments, the second segment 330 extends at least 99 cm in length from the side-hole 310. According to some embodiments, the second segment 330 extends at least 100 cm in length from the side-hole 310.

According to some embodiments, the second segment 330 extends at least 100.1 cm in length from the side-hole 310. According to some embodiments, the second segment 330 extends at least 100.2 cm in length from the side-hole 310. According to some embodiments, the second segment 330 extends at least 100.3 cm in length from the side-hole 310. According to some embodiments, the second segment 330 extends at least 100.4 cm in length from the side-hole 310. According to some embodiments, the second segment 330 extends at least 100.5 cm in length from the side-hole 310. According to some embodiments, the second segment 330 extends at least 100.6 cm in length from the side-hole 31100. According to some embodiments, the second segment 330 extends at least 100.7 cm in length from the side-hole 31100. According to some embodiments, the second segment 330 extends at least 100.8 cm in length from the side-hole 31100. According to some embodiments, the second segment 330 extends at least 100.9 cm in length from the side-hole 310. According to some embodiments, the second segment 330 extends at least 101 cm in length from the side-hole 310. According to some embodiments, the second segment 330 extends at least 102 cm in length from the side-hole 310. According to some embodiments, the second segment 330 extends at least 103 cm in length from the side-hole 310. According to some embodiments, the second segment 330 extends at least 104 cm in length from the side-hole 310. According to some embodiments, the second segment 330 extends at least 105 cm in length from the side-hole 310. According to some embodiments, the second segment 330 extends at least 106 cm in length from the side-hole 310. According to some embodiments, the second segment 330 extends at least 107 cm in length from the side-hole 310. According to some embodiments, the second segment 330 extends at least 108 cm in length from the side-hole 310. According to some embodiments, the second segment 330 extends at least 109 cm in length from the side-hole 310. According to some embodiments, the second segment 330 extends at least 110 cm in length from the side-hole 310. According to some embodiments, the second segment 330 extends at least 111 cm in length from the side-hole 310. According to some embodiments, the second segment 330 extends at least 112 cm in length from the side-hole 310. According to some embodiments, the second segment 330 extends at least 113 cm in length from the side-hole 310. According to some embodiments, the second segment 330 extends at least 114 cm in length from the side-hole 310. According to some embodiments, the second segment 330 extends at least 115 cm in length from the side-hole 310. According to some embodiments, the second segment 330 extends at least 116 cm in length from the side-hole 310. According to some embodiments, the second segment 330 extends at least 117 cm in length from the side-hole 310. According to some embodiments, the second segment 330 extends at least 118 cm in length from the side-hole 310. According to some embodiments, the second segment 330 extends at least 119 cm in length from the side-hole 310. According to some embodiments, the second segment 330 extends at least 120 cm in length from the side-hole 310. According to some embodiments, the second segment 330 extends at least 121 cm in length from the side-hole 310. According to some embodiments, the second segment 330 extends at least 122 cm in length from the side-hole 310. According to some embodiments, the second segment 330 extends at least 123 cm in length from the side-hole 310. According to some embodiments, the second segment 330 extends at least 124 cm in length from the side-hole 310. According to some embodiments, the second segment 330 extends at least 125 cm in length from the side-hole 310. According to some embodiments, the second segment 330 extends at least 126 cm in length from the side-hole 310. According to some embodiments, the second segment 330 extends at least 127 cm in length from the side-hole 310. According to some embodiments, the second segment 330 extends at least 128 cm in length from the side-hole 310. According to some embodiments, the second segment 330 extends at least 129 cm in length from the side-hole 310. According to some embodiments, the second segment 330 extends at least 130 cm in length from the side-hole 310. According to some embodiments, the second segment 330 extends at least 131 cm in length from the side-hole 310. According to some embodiments, the second segment 330 extends at least 132 cm in length from the side-hole 310. According to some embodiments, the second segment 330 extends at least 133 cm in length from the side-hole 310. According to some embodiments, the second segment 330 extends at least 134 cm in length from the side-hole 310. According to some embodiments, the second segment 330 extends at least 135 cm in length from the side-hole 310. According to some embodiments, the second segment 330 extends at least 136 cm in length from the side-hole 310. According to some embodiments, the second segment 330 extends at least 137 cm in length from the side-hole 310. According to some embodiments, the second segment 330 extends at least 138 cm in length from the side-hole 310. According to some embodiments, the second segment 330 extends at least 139 cm in length from the side-hole 310. According to some embodiments, the second segment 330 extends at least 140 cm in length from the side-hole 310. According to some embodiments, the second segment 330 extends at least 141 cm in length from the side-hole 310. According to some embodiments, the second segment 330 extends at least 142 cm in length from the side-hole 310. According to some embodiments, the second segment 330 extends at least 143 cm in length from the side-hole 310. According to some embodiments, the second segment 330 extends at least 144 cm in length from the side-hole 310. According to some embodiments, the second segment 330 extends at least 145 cm in length from the side-hole 310. According to some embodiments, the second segment 330 extends at least 146 cm in length from the side-hole 310. According to some embodiments, the second segment 330 extends at least 147 cm in length from the side-hole 310. According to some embodiments, the second segment 330 extends at least 148 cm in length from the side-hole 310. According to some embodiments, the second segment 330 extends at least 149 cm in length from the side-hole 310. According to some embodiments, the second segment 330 extends at least 150 cm in length from the side-hole 310. According to some embodiments, the second segment 330 extends at least 151 cm in length from the side-hole 310. According to some embodiments, the second segment 330 extends at least 152 cm in length from the side-hole 310. According to some embodiments, the second segment 330 extends at least 153 cm in length from the side-hole 310. According to some embodiments, the second segment 330 extends at least 154 cm in length from the side-hole 310. According to some embodiments, the second segment 330 extends at least 155 cm in length from the side-hole 310. According to some embodiments, the second segment 330 extends at least 156 cm in length from the side-hole 310. According to some embodiments, the second segment 330 extends at least 157 cm in length from the side-hole 310. According to some embodiments, the second segment 330 extends at least 158 cm in length from the side-hole 310. According to some embodiments, the second segment 330 extends at least 159 cm in length from the side-hole 310. According to some embodiments, the second segment 330 extends at least 160 cm in length from the side-hole 310.

According to some embodiments, the second segment 330 extends at least 161 cm in length from the side-hole 310. According to some embodiments, the second segment 330 extends at least 162 cm in length from the side-hole 310. According to some embodiments, the second segment 330 extends at least 163 cm in length from the side-hole 310. According to some embodiments, the second segment 330 extends at least 164 cm in length from the side-hole 310. According to some embodiments, the second segment 330 extends at least 165 cm in length from the side-hole 310. According to some embodiments, the second segment 330 extends at least 166 cm in length from the side-hole 310. According to some embodiments, the second segment 330 extends at least 167 cm in length from the side-hole 310. According to some embodiments, the second segment 330 extends at least 168 cm in length from the side-hole 310. According to some embodiments, the second segment 330 extends at least 169 cm in length from the side-hole 310. According to some embodiments, the second segment 330 extends at least 170 cm in length from the side-hole 310.

According to some embodiments, the second segment 330 extends at least 171 cm in length from the side-hole 310. According to some embodiments, the second segment 330 extends at least 172 cm in length from the side-hole 310. According to some embodiments, the second segment 330 extends at least 173 cm in length from the side-hole 310. According to some embodiments, the second segment 330 extends at least 174 cm in length from the side-hole 310. According to some embodiments, the second segment 330 extends at least 175 cm in length from the side-hole 310. According to some embodiments, the second segment 330 extends at least 176 cm in length from the side-hole 310. According to some embodiments, the second segment 330 extends at least 177 cm in length from the side-hole 310. According to some embodiments, the second segment 330 extends at least 178 cm in length from the side-hole 310. According to some embodiments, the second segment 330 extends at least 179 cm in length from the side-hole 310. According to some embodiments, the second segment 330 extends at least 180 cm in length from the side-hole 310.

According to some embodiments, the second segment 330 extends at least 181 cm in length from the side-hole 310. According to some embodiments, the second segment 330 extends at least 182 cm in length from the side-hole 310. According to some embodiments, the second segment 330 extends at least 183 cm in length from the side-hole 310. According to some embodiments, the second segment 330 extends at least 184 cm in length from the side-hole 310. According to some embodiments, the second segment 330 extends at least 185 cm in length from the side-hole 310. According to some embodiments, the second segment 330 extends at least 186 cm in length from the side-hole 310. According to some embodiments, the second segment 330 extends at least 187 cm in length from the side-hole 310. According to some embodiments, the second segment 330 extends at least 188 cm in length from the side-hole 310. according to some embodiments, the second segment 330 extends at least 189 cm in length from the side-hole 310. According to some embodiments, the second segment 330 extends at least 190 cm in length from the side-hole 310.

According to some embodiments, the second segment 330 extends at least 191 cm in length from the side-hole 310. According to some embodiments, the second segment 330 extends at least 192 cm in length from the side-hole 310. According to some embodiments, the second segment 330 extends at least 193 cm in length from the side-hole 310. According to some embodiments, the second segment 330 extends at least 194 cm in length from the side-hole 310. According to some embodiments, the second segment 330 extends at least 195 cm in length from the side-hole 310. According to some embodiments, the second segment 330 extends at least 196 cm in length from the side-hole 310. According to some embodiments, the second segment 330 extends at least 197 cm in length from the side-hole 310. According to some embodiments, the second segment 330 extends at least 198 cm in length from the side-hole 310. According to some embodiments, the second segment 330 extends at least 199 cm in length from the side-hole 310. According to some embodiments, the second segment 330 extends at least 200 cm in length from the side-hole 310.

According to some embodiments, the diameter of the lumen of the first segment 320 ranges from about 0.1 French (Fr) to about 30 French (Fr). According to some embodiments, the diameter of the lumen 370 of the first segment 320 ranges from about 0.1 French (Fr) to at least 26 French (Fr). According to some embodiments, the diameter of the lumen 370 of the first segment 320 ranges from at least 0.1 French (Fr) to at least 26 French (Fr). According to some embodiments, the diameter of the lumen 370 of first segment 320 is at least 0.1 French (Fr). According to some embodiments, the diameter of the lumen 370 of first segment 320 is at least 0.2 French (Fr). According to some embodiments, the diameter of the lumen 370 of first segment 320 is at least 0.3 French (Fr). According to some embodiments, the diameter of the lumen 370 of first segment 320 is at least 0.4 French (Fr). According to some embodiments, the diameter of the lumen 370 of first segment 320 is at least 0.5 French (Fr). According to some embodiments, the diameter of the lumen 370 of first segment 320 is at least 0.6 French (Fr). According to some embodiments, the diameter of the lumen 370 of first segment 320 is at least 0.7 French (Fr). According to some embodiments, the diameter of the lumen 370 of first segment 320 is at least 0.8 French (Fr). According to some embodiments, the diameter of the lumen 370 of first segment 320 is at least 0.9 French (Fr). According to some embodiments, the diameter of the lumen 370 of first segment 320 is at least 1 French (Fr). According to some embodiments, the diameter of the lumen 370 of first segment 320 is at least 2 French (Fr). According to some embodiments, the diameter of the lumen 370 of first segment 320 is at least 3 French (Fr). According to some embodiments, the diameter of the lumen 370 of first segment 320 is at least 4 French (Fr). According to some embodiments, the diameter of the lumen 370 of first segment 320 is at least 5 French (Fr). According to some embodiments, the diameter of the lumen 370 of first segment 320 is at least 6 French (Fr). According to some embodiments, the diameter of the lumen 370 of first segment 320 is at least 7 French (Fr). According to some embodiments, the diameter of the lumen 370 of first segment 320 is at least 8 French (Fr). According to some embodiments, the diameter of the lumen 370 of first segment 320 is at least 9 French (Fr). According to some embodiments, the diameter of the lumen 370 of first segment 320 is at least 10 French (Fr). According to some embodiments, the diameter of the lumen 370 of first segment 320 is at least 11 French (Fr). According to some embodiments, the diameter of the lumen 370 of first segment 320 is at least 12 French (Fr). According to some embodiments, the diameter of the lumen 370 of first segment 320 is at least 13 French (Fr). According to some embodiments, the diameter of the lumen 370 of first segment 320 is at least 14 French (Fr). According to some embodiments, the diameter of the lumen 370 of first segment 320 is at least 15 French (Fr). According to some embodiments, the diameter of the lumen 370 of first segment 320 is at least 16 French (Fr). According to some embodiments, the diameter of the lumen 370 of first segment 320 is at least 17 French (Fr). According to some embodiments, the diameter of the lumen 370 of first segment 320 is at least 18 French (Fr). According to some embodiments, the diameter of the lumen 370 of first segment 320 is at least 19 French (Fr). According to some embodiments, the diameter of the lumen 370 of first segment 320 is at least 20 French (Fr). According to some embodiments, the diameter of the lumen 370 of first segment 320 is at least 21 French (Fr). According to some embodiments, the diameter of the lumen 370 of first segment 320 is at least 22 French (Fr). According to some embodiments, the diameter of the lumen 370 of first segment 320 is at least 23 French (Fr). According to some embodiments, the diameter of the lumen 370 of first segment 320 is at least 24 French (Fr). According to some embodiments, the diameter of the lumen 370 of first segment 320 is at least 25 French (Fr). According to some embodiments, the diameter of the lumen 370 of first segment 320 is at least 26 French (Fr). According to some embodiments, the diameter of the lumen 370 of first segment 320 is at least 27 French (Fr). According to some embodiments, the diameter of the lumen 370 of first segment 320 is at least 28 French (Fr). According to some embodiments, the diameter of the lumen 370 of first segment 320 is at least 29 French (Fr). According to some embodiments, the diameter of the lumen 370 of first segment 320 is about 30 French (Fr).

According to some embodiments, the diameter of the lumen 370 of first segment 320 is less than 1 French (Fr). According to some embodiments the diameter of the lumen 370 of first segment 320 ranges from at least 0.0152 cm (at least 0.006 inches) to at least 0.0508 cm (at least 0.02 inches). According to some embodiments, the diameter of the lumen 370 of first segment 320 is at least 0.0254 cm (at least 0.010 inches). According to some embodiments, the diameter of the lumen 370 of first segment 320 is at least 0.0279 cm (at least 0.011 inches). According to some embodiments, the diameter of the lumen 370 of first segment 320 is at least 0.0305 cm (at least 0.012 inches). According to some embodiments, the diameter of the lumen 370 of first segment 320 is at least 0.0356 cm (at least 0.014 inches). According to some embodiments, the diameter of the lumen 370 of first segment 320 is at least 0.0406 cm (at least 0.016 inches). According to some embodiments, the diameter of the lumen 370 of first segment 320 is at least 0.0457 cm (at least 0.018 inches). According to some embodiments, the diameter of the lumen 370 of first segment 320 is at least 0.0508 cm (at least 0.02 inches).

According to some embodiments, the diameter of the lumen 370 of the second segment 330 ranges from about 0.1 French (Fr) to at least 30 French (Fr). According to some embodiments, the diameter of the lumen 370 of the second segment 330 is at least 0.1 French (Fr). According to some embodiments, the diameter of the lumen 370 of the second segment 330 is at least 0.2 French (Fr). According to some embodiments, the diameter of the lumen 370 of the second segment 330 is at least 0.3 French (Fr). According to some embodiments, the diameter of the lumen 370 of the second segment 330 is at least 0.4 French (Fr). According to some embodiments, the diameter of the lumen 370 of the second segment 330 is at least 0.5 French (Fr). According to some embodiments, the diameter of the lumen 370 of the second segment 330 is at least 0.6 French (Fr). According to some embodiments, the diameter of the lumen 370 of the second segment 330 is at least 0.7 French (Fr). According to some embodiments, the diameter of the lumen 370 of the second segment 330 is at least 0.8 French (Fr). According to some embodiments, the diameter of the lumen 370 of the second segment 330 is at least 0.9 French (Fr). According to some embodiments, the d iameterof the lumen 370 of the second segment 330 of the endovascular device is at least 1.0 Fr. According to some embodiments, the diameter of the lumen 370 of the second segment 330 of the endovascular device is at least 1.1 French (Fr) (0.014 inches). According to some embodiments, the diameter of the lumen 370 of the second segment 330 of the endovascular device is at least 1.2 French (Fr) (0.016 inches). According to some embodiments, the diameter of the lumen 370 of the second segment 330 of the endovascular device is at least 1.3 French (Fr) (0.017 inches). According to some embodiments, the diameter of the lumen 370 of the second segment 330 of the endovascular device is at least 1.4 French (Fr) (0.018 inches). According to some embodiments, the diameter of the lumen 370 of the second segment 330 of the endovascular device is at least 1.5 French (Fr) (0.02 inches). According to some embodiments, the diameter of the lumen 370 of the second segment 330 of the endovascular device is at least 1.6 French (Fr) (0.021 inches). According to some embodiments, the diameter of the lumen 370 of the second segment 330 of the endovascular device is at least 1.7 French (Fr) (0.022 inches). According to some embodiments, the diameter of the lumen 370 of the second segment 330 of the endovascular device is at least 1.8 French (Fr) (0.024 inches). According to some embodiments, the diameter of the lumen 370 of the second segment 330 of the endovascular device is at least 1.9 French (Fr) (0.025 inches). According to some embodiments, the diameter of the lumen 370 of the second segment 330 of the endovascular device is at least 2 French (Fr) (0.026 inches). According to some embodiments, the diameter of the lumen 370 of the second segment 330 of the endovascular device is at least 2.1 French (Fr) (0.028 inches). According to some embodiments, the diameter of the lumen 370 of the second segment 330 of the endovascular device is at least 2.2 French (Fr) (0.029 inches). According to some embodiments, the diameter of the lumen 370 of the second segment 330 of the endovascular device is at least 2.3 French (Fr) (0.03 inches). According to some embodiments, the diameter of the lumen 370 of the second segment 330 of the endovascular device is at least 2.4 French (Fr) (0.031 inches). According to some embodiments, the diameter of the lumen 370 of the second segment 330 of the endovascular device is at least 2.5 French (Fr) (0.033 inches). According to some embodiments, the diameter of the lumen 370 of the second segment 330 of the endovascular device is at least 2.6 French (Fr) (0.034 inches). According to some embodiments, the diameter of the lumen 370 of the second segment 330 of the endovascular device is at least 2.7 French (Fr) (0.035 inches). According to some embodiments, the diameter of the lumen 370 of the second segment 330 of the endovascular device is at least 2.8 French (Fr) (0.037 inches). According to some embodiments, the diameter of the lumen 370 of the second segment 330 of the endovascular device is at least 2.9 French (Fr) (0.038 inches). According to some embodiments, the diameter of the lumen 370 of the second segment 330 of the endovascular device is at least 3 French (Fr) (0.039 inches). According to some embodiments, the diameter of the lumen 370 of the second segment 330 of the endovascular device is at least 3.1 French (Fr) (0.041 inches). According to some embodiments, the diameter of the lumen 370 of the second segment 330 of the endovascular device is at least 3.2 French (Fr) (0.042 inches). According to some embodiments, the diameter of the lumen 370 of the second segment 330 of the endovascular device is at least 3.3 French (Fr) (0.043 inches). According to some embodiments, the diameter of the lumen 370 of the second segment 330 of the endovascular device is at least 3.4 French (Fr) (0.045 inches). According to some embodiments, the diameter of the lumen 370 of the second segment 330 of the endovascular device is at least 3.5 French (Fr) (0.046 inches). According to some embodiments, the diameter of the lumen 370 of the second segment 330 of the endovascular device is at least 3.6 French (Fr) (0.047 inches). According to some embodiments, the diameter of the lumen 370 of the second segment 330 of the endovascular device is at least 3.7 French (Fr) (0.049 inches). According to some embodiments, the diameter of the lumen 370 of the second segment 330 of the endovascular device is at least 3.8 French (Fr) (0.05 inches). According to some embodiments, the diameter of the lumen 370 of the second segment 330 of the endovascular device is at least 3.9 French (Fr) (0.051 inches). According to some embodiments, the diameter of the lumen 370 of the second segment 330 of the endovascular device is at least 4 French (Fr) (0.052 inches). According to some embodiments, the diameter of the lumen 370 of the second segment 330 of the endovascular device is at least 4.1 French (Fr) (0.054 inches). According to some embodiments, the diameter of the lumen 370 of the second segment 330 of the endovascular device is at least 4.2 French (Fr) (0.055 inches). According to some embodiments, the diameter of the lumen 370 of the second segment 330 of the endovascular device is at least 4.3 French (Fr) (0.056 inches). According to some embodiments, the diameter of the lumen 370 of the second segment 330 of the endovascular device is at least 4.4 French (Fr) (0.058 inches). According to some embodiments, the diameter of the lumen 370 of the second segment 330 of the endovascular device is at least 4.5 French (Fr) (0.059 inches). According to some embodiments, the diameter of the lumen 370 of the second segment 330 of the endovascular device is at least 4.6 French (Fr) (0.06 inches). According to some embodiments, the diameter of the lumen 370 of the second segment 330 of the endovascular device is at least 4.7 French (Fr) (0.062 inches). According to some embodiments, the diameter of the lumen 370 of the second segment 330 of the endovascular device is at least 4.8 French (Fr) (0.063 inches). According to some embodiments, the diameter of the lumen 370 of the second segment 330 of the endovascular device is at least 4.9 French (Fr) (0.064 inches). According to some embodiments, the diameter of the lumen 370 of the second segment 330 of the endovascular device is at least 5 French (Fr) (0.066 inches). According to some embodiments, the diameter of the lumen 370 of the second segment 330 of the endovascular device is at least 5.1 French (Fr) (0.067 inches). According to some embodiments, the diameter of the lumen 370 of the second segment 330 of the endovascular device is at least 5.2 French (Fr) (0.068 inches). According to some embodiments, the diameter of the lumen 370 of the second segment 330 of the endovascular device is at least 5.3 French (Fr) (0.070 inches). According to some embodiments, the diameter of the lumen 370 of the second segment 330 of the endovascular device is at least 5.4 French (Fr) (0.071 inches). According to some embodiments, the diameter of the lumen 370 of the second segment 330 of the endovascular device is at least 5.5 French (Fr) (0.072 inches). According to some embodiments, the diameter of the lumen 370 of the second segment 330 of the endovascular device is at least 5.6 French (Fr) (0.073 inches). According to some embodiments, the diameter of the lumen 370 of the second segment 330 of the endovascular device is at least 5.7 French (Fr) (0.075 inches). According to some embodiments, the diameter of the lumen 370 of the second segment 330 of the endovascular device is at least 5.8 French (Fr) (0.076 inches). According to some embodiments, the diameter of the lumen 370 of the second segment 330 of the endovascular device is at least 5.9 French (Fr) (0.077 inches). According to some embodiments, the diameter of the lumen 370 of the second segment 330 of the endovascular device is at least 6 French (Fr) (0.079 inches). According to some embodiments, the diameter of the lumen 370 of the second segment 330 of the endovascular device is at least 6.1 French (Fr) (0.080 inches). According to some embodiments, the diameter of the lumen 370 of the second segment 330 of the endovascular device is at least 6.2 French (Fr) (0.081 inches). According to some embodiments, the diameter of the lumen 370 of the second segment 330 of the endovascular device is at least 6.3 French (Fr) (0.083 inches). According to some embodiments, the diameter of the lumen 370 of the second segment 330 of the endovascular device is at least 6.4 French (Fr) (0.084 inches). According to some embodiments, the diameter of the lumen 370 of the second segment 330 of the endovascular device is at least 6.5 French (Fr) (0.085 inches). According to some embodiments, the diameter of the lumen 370 of the second segment 330 of the endovascular device is at least 6.6 French (Fr) (0.087 inches). According to some embodiments, the diameter of the lumen 370 of the second segment 330 of the endovascular device is at least 6.7 French (Fr) (0.088 inches). According to some embodiments, the diameter of the lumen 370 of the second segment 330 of the endovascular device is at least 6.8 French (Fr) (0.089 inches). According to some embodiments, the diameter of the lumen 370 of the second segment 330 of the endovascular device is at least 6.9 French (Fr) (0.091 inches). According to some embodiments, the diameter of the lumen 370 of the second segment 330 of the endovascular device is at least 7 French (Fr) (0.092 inches). According to some embodiments, the diameter of the lumen 370 of the second segment 330 of the endovascular device is at least 7.1 French (Fr). According to some embodiments, the diameter of the lumen 370 of the second segment 330 of the endovascular device is at least 7.2 French (Fr). According to some embodiments, the diameter of the lumen 370 of the second segment 330 of the endovascular device is at least 7.3 French (Fr). According to some embodiments, the diameter of the lumen 370 of the second segment 330 of the endovascular device is at least 7.4 French (Fr). According to some embodiments, the diameter of the lumen 370 of the second segment 330 of the endovascular device is at least 7.5 French (Fr). According to some embodiments, the diameter of the lumen 370 of the second segment 330 of the endovascular device is at least 7.6 French (Fr). According to some embodiments, the diameter of the lumen 370 of the second segment 330 of the endovascular device is at least 7.7 French (Fr). According to some embodiments, the diameter of the lumen 370 of the second segment 330 of the endovascular device is at least 7.8 French (Fr). According to some embodiments, the diameter of the lumen 370 of the second segment 330 of the endovascular device is at least 7.9 French (Fr). According to some embodiments, the diameter of the lumen 370 of the second segment 330 of the endovascular device is at least 8.0 French (Fr). According to some embodiments, the diameter of the lumen 370 of the second segment 330 of the endovascular device is at least 8.1 French (Fr). According to some embodiments, the diameter of the lumen 370 of the second segment 330 of the endovascular device is at least 8.2 French (Fr). According to some embodiments, the diameter of the lumen 370 of the second segment 330 of the endovascular device is at least 8.3 French (Fr). According to some embodiments, the diameter of the lumen 370 of the second segment 330 of the endovascular device is at least 8.4 French (Fr). According to some embodiments, the diameter of the lumen 370 of the second segment 330 of the endovascular device is at least 8.5 French (Fr). According to some embodiments, the diameter of the lumen 370 of the second segment 330 of the endovascular device is at least 8.6 French (Fr). According to some embodiments, the diameter of the lumen 370 of the second segment 330 of the endovascular device is at least 8.7 French (Fr). According to some embodiments, the diameter of the lumen 370 of the second segment 330 of the endovascular device is at least 8.8 French (Fr). According to some embodiments, the diameter of the lumen 370 of the second segment 330 of the endovascular device is at least 8.9 French (Fr). According to some embodiments, the diameter of the lumen 370 of the second segment 330 of the endovascular device is at least 9.0 French (Fr). According to some embodiments, the diameter of the lumen 370 of the second segment 330 of the endovascular device is at least 9.1 French (Fr). According to some embodiments, the diameter of the lumen 370 of the second segment 330 of the endovascular device is at least 9.2 French (Fr). According to some embodiments, the diameter of the lumen 370 of the second segment 330 of the endovascular device is at least 9.3 French (Fr). According to some embodiments, the diameter of the lumen 370 of the second segment 330 of the endovascular device is at least 9.4 French (Fr). According to some embodiments, the diameter of the lumen 370 of the second segment 330 of the endovascular device is at least 9.5 French (Fr). According to some embodiments, the diameter of the lumen 370 of the second segment 330 of the endovascular device is at least 9.6 French (Fr). According to some embodiments, the diameter of the lumen 370 of the second segment 330 of the endovascular device is at least 9.7 French (Fr). According to some embodiments, the diameter of the lumen 370 of the second segment 330 of the endovascular device is at least 9.8 French (Fr). According to some embodiments, the diameter of the lumen 370 of the second segment 330 of the endovascular device is at least 9.9 French (Fr). According to some embodiments, the diameter of the lumen 370 of the second segment 330 of the endovascular device is at least 10.0 French (Fr). According to some embodiments, the diameter of the lumen 370 of the second segment 330 of the endovascular device is at least 10.1 French (Fr). According to some embodiments, the diameter of the lumen 370 of the second segment 330 of the endovascular device is at least 10.2 French (Fr). According to some embodiments, the diameter of the lumen 370 of the second segment 330 of the endovascular device is at least 10.3 French (Fr). According to some embodiments, the diameter of the lumen 370 of the second segment 330 of the endovascular device is at least 10.4 French (Fr). According to some embodiments, the diameter of the lumen 370 of the second segment 330 of the endovascular device is at least 10.5 French (Fr). According to some embodiments, the diameter of the lumen 370 of the second segment 330 of the endovascular device is at least 10.6 French (Fr). According to some embodiments, the diameter of the lumen 370 of the second segment 330 of the endovascular device is at least 10.7 French (Fr). According to some embodiments, the diameter of the lumen 370 of the second segment 330 of the endovascular device is at least 10.8 French (Fr). According to some embodiments, the diameter of the lumen 370 of the second segment 330 of the endovascular device is at least 10.9 French (Fr). According to some embodiments, the diameter of the lumen 370 of the second segment 330 of the endovascular device is at least 11.0 French (Fr). According to some embodiments, the diameter of the lumen 370 of the second segment 330 of the endovascular device is at least 11.1 French (Fr). According to some embodiments, the diameter of the lumen 370 of the second segment 330 of the endovascular device is at least 11.2 French (Fr). According to some embodiments, the diameter of the lumen 370 of the second segment 330 of the endovascular device is at least 11.3 French (Fr). According to some embodiments, the diameter of the lumen 370 of the second segment 330 of the endovascular device is at least 11.4 French (Fr). According to some embodiments, the diameter of the lumen 370 of the second segment 330 of the endovascular device is at least 11.5 French (Fr). According to some embodiments, the diameter of the lumen 370 of the second segment 330 of the endovascular device is at least 11.6 French (Fr). According to some embodiments, the diameter of the lumen 370 of the second segment 330 of the endovascular device is at least 11.7 French (Fr). According to some embodiments, the diameter of the lumen 370 of the second segment 330 of the endovascular device is at least 11.8 French (Fr). According to some embodiments, the diameter of the lumen 370 of the second segment 330 of the endovascular device is at least 11.9 French (Fr). According to some embodiments, the diameter of the lumen 370 of the second segment 330 of the endovascular device is at least 12.0 French (Fr). According to some embodiments, the diameter of the lumen 370 of the second segment 330 of the endovascular device is at least 12.1 French (Fr). According to some embodiments, the diameter of the lumen 370 of the second segment 330 of the endovascular device is at least 12.2 French (Fr). According to some embodiments, the diameter of the lumen 370 of the second segment 330 of the endovascular device is at least 12.3 French (Fr). According to some embodiments, the diameter of the lumen 370 of the second segment 330 of the endovascular device is at least 12.4 French (Fr). According to some embodiments, the diameter of the lumen 370 of the second segment 330 of the endovascular device is at least 12.5 French (Fr). According to some embodiments, the diameter of the lumen 370 of the second segment 330 of the endovascular device is at least 12.6 French (Fr). According to some embodiments, the diameter of the lumen 370 of the second segment 330 of the endovascular device is at least 12.7 French (Fr). According to some embodiments, the diameter of the lumen 370 of the second segment 330 of the endovascular device is at least 12.8 French (Fr). According to some embodiments, the diameter of the lumen 370 of the second segment 330 of the endovascular device is at least 12.9 French (Fr). According to some embodiments, the diameter of the lumen 370 of the second segment 330 of the endovascular device is at least 13.0 French (Fr). According to some embodiments, the diameter of the lumen 370 of the second segment 330 of the endovascular device is at least 13.1 French (Fr). According to some embodiments, the diameter of the lumen 370 of the second segment 330 of the endovascular device is at least 13.2 French (Fr). According to some embodiments, the diameter of the lumen 370 of the second segment 330 of the endovascular device is at least 13.3 French (Fr). According to some embodiments, the diameter of the lumen 370 of the second segment 330 of the endovascular device is at least 13.4 French (Fr). According to some embodiments, the diameter of the lumen 370 of the second segment 330 of the endovascular device is at least 13.5 French (Fr). According to some embodiments, the diameter of the lumen 370 of the second segment 330 of the endovascular device is at least 13.6 French (Fr). According to some embodiments, the diameter of the lumen 370 of the second segment 330 of the endovascular device is at least 13.7 French (Fr). According to some embodiments, the diameter of the lumen 370 of the second segment 330 of the endovascular device is at least 13.8 French (Fr). According to some embodiments, the diameter of the lumen 370 of the second segment 330 of the endovascular device is at least 13.9 French (Fr). According to some embodiments, the diameter of the lumen 370 of the second segment 330 of the endovascular device is at least 14.0 French (Fr). According to some embodiments, the diameter of the lumen 370 of the second segment 330 of the endovascular device is at least 14.1 French (Fr). According to some embodiments, the diameter of the lumen 370 of the second segment 330 of the endovascular device is at least 14.2 French (Fr). According to some embodiments, the diameter of the lumen 370 of the second segment 330 of the endovascular device is at least 14.3 French (Fr). According to some embodiments, the diameter of the lumen 370 of the second segment 330 of the endovascular device is at least 14.4 French (Fr). According to some embodiments, the diameter of the lumen 370 of the second segment 330 of the endovascular device is at least 14.5 French (Fr). According to some embodiments, the diameter of the lumen 370 of the second segment 330 of the endovascular device is at least 14.6 French (Fr). According to some embodiments, the diameter of the lumen 370 of the second segment 330 of the endovascular device is at least 14.7 French (Fr). According to some embodiments, the diameter of the lumen 370 of the second segment 330 of the endovascular device is at least 14.8 French (Fr). According to some embodiments, the diameter of the lumen 370 of the second segment 330 of the endovascular device is at least 14.9 French (Fr). According to some embodiments, the diameter of the lumen 370 of the second segment 330 of the endovascular device is at least 15.0 French (Fr). According to some embodiments, the diameter of the lumen 370 of the second segment 330 of the endovascular device is at least 15.0 French (Fr). According to some embodiments, the diameter of the lumen 370 of the second segment 330 of the endovascular device is at least 15.1 French (Fr). According to some embodiments, the diameter of the lumen 370 of the second segment 330 of the endovascular device is at least 15.2 French (Fr). According to some embodiments, the diameter of the lumen 370 of the second segment 330 of the endovascular device is at least 15.3 French (Fr). According to some embodiments, the diameter of the lumen 370 of the second segment 330 of the endovascular device is at least 15.4 French (Fr). According to some embodiments, the diameter of the lumen 370 of the second segment 330 of the endovascular device is at least 15.5 French (Fr). According to some embodiments, the diameter of the lumen 370 of the second segment 330 of the endovascular device is at least 15.6 French (Fr). According to some embodiments, the diameter of the lumen 370 of the second segment 330 of the endovascular device is at least 15.7 French (Fr). According to some embodiments, the diameter of the lumen 370 of the second segment 330 of the endovascular device is at least 15.8 French (Fr). According to some embodiments, the diameter of the lumen 370 of the second segment 330 of the endovascular device is at least 15.9 French (Fr). According to some embodiments, the diameter of the lumen 370 of the second segment 330 of the endovascular device is at least 16.0 French (Fr). According to some embodiments, the diameter of the lumen 370 of the second segment 330 of the endovascular device is at least 16.1 French (Fr). According to some embodiments, the diameter of the lumen 370 of the second segment 330 of the endovascular device is at least 16.2 French (Fr). According to some embodiments, the diameter of the lumen 370 of the second segment 330 of the endovascular device is at least 16.3 French (Fr). According to some embodiments, the diameter of the lumen 370 of the second segment 330 of the endovascular device is at least 16.4 French (Fr). According to some embodiments, the diameter of the lumen 370 of the second segment 330 of the endovascular device is at least 16.5 French (Fr). According to some embodiments, the diameter of the lumen 370 of the second segment 330 of the endovascular device is at least 16.6 French (Fr). According to some embodiments, the diameter of the lumen 370 of the second segment 330 of the endovascular device is at least 16.7 French (Fr). According to some embodiments, the diameter of the lumen 370 of the second segment 330 of the endovascular device is at least 16.8 French (Fr). According to some embodiments, the diameter of the lumen 370 of the second segment 330 of the endovascular device is at least 16.9 French (Fr). According to some embodiments, the diameter of the lumen 370 of the second segment 330 of the endovascular device is at least 17.0 French (Fr). According to some embodiments, the diameter of the lumen 370 of the second segment 330 of the endovascular device is at least 17.1 French (Fr). According to some embodiments, the diameter of the lumen 370 of the second segment 330 of the endovascular device is at least 17.2 French (Fr). According to some embodiments, the diameter of the lumen 370 of the second segment 330 of the endovascular device is at least 17.3 French (Fr). According to some embodiments, the diameter of the lumen 370 of the second segment 330 of the endovascular device is at least 17.4 French (Fr). According to some embodiments, the diameter of the lumen 370 of the second segment 330 of the endovascular device is at least 17.5 French (Fr). According to some embodiments, the diameter of the lumen 370 of the second segment 330 of the endovascular device is at least 17.6 French (Fr). According to some embodiments, the diameter of the lumen 370 of the second segment 330 of the endovascular device is at least 17.7 French (Fr). According to some embodiments, the diameter of the lumen 370 of the second segment 330 of the endovascular device is at least 17.8 French (Fr). According to some embodiments, the diameter of the lumen 370 of the second segment 330 of the endovascular device is at least 17.9 French (Fr). According to some embodiments, the diameter of the lumen 370 of the second segment 330 of the endovascular device is at least 18.0 French (Fr). According to some embodiments, the diameter of the lumen 370 of the second segment 330 of the endovascular device is at least 18.1 French (Fr). According to some embodiments, the diameter of the lumen 370 of the second segment 330 of the endovascular device is at least 18.2 French (Fr). According to some embodiments, the diameter of the lumen 370 of the second segment 330 of the endovascular device is at least 18.3 French (Fr). According to some embodiments, the diameter of the lumen 370 of the second segment 330 of the endovascular device is at least 18.4 French (Fr). According to some embodiments, the diameter of the lumen 370 of the second segment 330 of the endovascular device is at least 18.5 French (Fr). According to some embodiments, the diameter of the lumen 370 of the second segment 330 of the endovascular device is at least 18.6 French (Fr). According to some embodiments, the diameter of the lumen 370 of the second segment 330 of the endovascular device is at least 18.7 French (Fr). According to some embodiments, the diameter of the lumen 370 of the second segment 330 of the endovascular device is at least 18.8 French (Fr). According to some embodiments, the diameter of the lumen 370 of the second segment 330 of the endovascular device is at least 18.9 French (Fr). According to some embodiments, the diameter of the lumen 370 of the second segment 330 of the endovascular device is at least 19.0 French (Fr). According to some embodiments, the diameter of the lumen 370 of the second segment 330 of the endovascular device is at least 19.1 French (Fr). According to some embodiments, the diameter of the lumen 370 of the second segment 330 of the endovascular device is at least 19.2 French (Fr). According to some embodiments, the diameter of the lumen 370 of the second segment 330 of the endovascular device is at least 19.3 French (Fr). According to some embodiments, the diameter of the lumen 370 of the second segment 330 of the endovascular device is at least 19.4 French (Fr). According to some embodiments, the diameter of the lumen 370 of the second segment 330 of the endovascular device is at least 19.5 French (Fr). According to some embodiments, the diameter of the lumen 370 of the second segment 330 of the endovascular device is at least 19.6 French (Fr). According to some embodiments, the diameter of the lumen 370 of the second segment 330 of the endovascular device is at least 19.6 French (Fr). According to some embodiments, the diameter of the lumen 370 of the second segment 330 of the endovascular device is at least 19.7 French (Fr). According to some embodiments, the diameter of the lumen 370 of the second segment 330 of the endovascular device is at least 19.8 French (Fr). According to some embodiments, the diameter of the lumen 370 of the second segment 330 of the endovascular device is at least 19.9 French (Fr). According to some embodiments, the diameter of the lumen 370 of the second segment 330 of the endovascular device is at least 20.0 French (Fr). According to some embodiments, the diameter of the lumen 370 of the second segment 330 of the endovascular device is at least 20.1 French (Fr). According to some embodiments, the diameter of the lumen 370 of the second segment 330 of the endovascular device is at least 20.2 French (Fr). According to some embodiments, the diameter of the lumen 370 of the second segment 330 of the endovascular device is at least 20.3 French (Fr). According to some embodiments, the diameter of the lumen 370 of the second segment 330 of the endovascular device is at least 20.4 French (Fr). According to some embodiments, the diameter of the lumen 370 of the second segment 330 of the endovascular device is at least 20.5 French (Fr). According to some embodiments, the diameter of the lumen 370 of the second segment 330 of the endovascular device is at least 20.6 French (Fr). According to some embodiments, the diameter of the lumen 370 of the second segment 330 of the endovascular device is at least 20.7 French (Fr). According to some embodiments, the diameter of the lumen 370 of the second segment 330 of the endovascular device is at least 20.8 French (Fr). According to some embodiments, the diameter of the lumen 370 of the second segment 330 of the endovascular device is at least 20.9 French (Fr). According to some embodiments, the diameter of the lumen 370 of the second segment 330 of the endovascular device is at least 21.0 French (Fr). According to some embodiments, the diameter of the lumen 370 of the second segment 330 of the endovascular device is at least 21.1 French (Fr). According to some embodiments, the diameter of the lumen 370 of the second segment 330 of the endovascular device is at least 21.2 French (Fr). According to some embodiments, the diameter of the lumen 370 of the second segment 330 of the endovascular device is at least 21.3 French (Fr). According to some embodiments, the diameter of the lumen 370 of the second segment 330 of the endovascular device is at least 21.4 French (Fr). According to some embodiments, the diameter of the lumen 370 of the second segment 330 of the endovascular device is at least 21.5 French (Fr). According to some embodiments, the diameter of the lumen 370 of the second segment 330 of the endovascular device is at least 21.6 French (Fr). According to some embodiments, the diameter of the lumen 370 of the second segment 330 of the endovascular device is at least 21.7 French (Fr). According to some embodiments, the diameter of the lumen 370 of the second segment 330 of the endovascular device is at least 21.8 French (Fr). According to some embodiments, the diameter of the lumen 370 of the second segment 330 of the endovascular device is at least 21.9 French (Fr). According to some embodiments, the diameter of the lumen 370 of the second segment 330 of the endovascular device is at least 22.0 French (Fr). According to some embodiments, the diameter of the lumen 370 of the second segment 330 of the endovascular device is at least 22.1 French (Fr). According to some embodiments, the diameter of the lumen 370 of the second segment 330 of the endovascular device is at least 22.2 French (Fr). According to some embodiments, the diameter of the lumen 370 of the second segment 330 of the endovascular device is at least 22.3 French (Fr). According to some embodiments, the diameter of the lumen 370 of the second segment 330 of the endovascular device is at least 22.4 French (Fr). According to some embodiments, the diameter of the lumen 370 of the second segment 330 of the endovascular device is at least 22.5 French (Fr). According to some embodiments, the diameter of the lumen 370 of the second segment 330 of the endovascular device is at least 22.6 French (Fr). According to some embodiments, the diameter of the lumen 370 of the second segment 330 of the endovascular device is at least 22.7 French (Fr). According to some embodiments, the diameter of the lumen 370 of the second segment 330 of the endovascular device is at least 22.8 French (Fr). According to some embodiments, the diameter of the lumen 370 of the second segment 330 of the endovascular device is at least 22.9 French (Fr). According to some embodiments, the diameter of the lumen 370 of the second segment 330 of the endovascular device is at least 23.0 French (Fr). According to some embodiments, the diameter of the lumen 370 of the second segment 330 of the endovascular device is at least 23.1 French (Fr). According to some embodiments, the diameter of the lumen 370 of the second segment 330 of the endovascular device is at least 23.2 French (Fr). According to some embodiments, the diameter of the lumen 370 of the second segment 330 of the endovascular device is at least 23.3 French (Fr). According to some embodiments, the diameter of the lumen 370 of the second segment 330 of the endovascular device is at least 23.4 French (Fr). According to some embodiments, the diameter of the lumen 370 of the second segment 330 of the endovascular device is at least 23.5 French (Fr). According to some embodiments, the diameter of the lumen 370 of the second segment 330 of the endovascular device is at least 23.6 French (Fr). According to some embodiments, the diameter of the lumen 370 of the second segment 330 of the endovascular device is at least 23.7 French (Fr). According to some embodiments, the diameter of the lumen 370 of the second segment 330 of the endovascular device is at least 23.8 French (Fr). According to some embodiments, the diameter of the lumen 370 of the second segment 330 of the endovascular device is at least 23.9 French (Fr). According to some embodiments, the diameter of the lumen 370 of the second segment 330 of the endovascular device is at least 24.0 French (Fr). According to some embodiments, the diameter of the lumen 370 of the second segment 330 of the endovascular device is at least 24.1 French (Fr). According to some embodiments, the diameter of the lumen 370 of the second segment 330 of the endovascular device is at least 24.2 French (Fr). According to some embodiments, the diameter of the lumen 370 of the second segment 330 of the endovascular device is at least 24.3 French (Fr). According to some embodiments, the diameter of the lumen 370 of the second segment 330 of the endovascular device is at least 24.4 French (Fr). According to some embodiments, the diameter of the lumen 370 of the second segment 330 of the endovascular device is at least 24.5 French (Fr). According to some embodiments, the diameter of the lumen 370 of the second segment 330 of the endovascular device is at least 24.6 French (Fr). According to some embodiments, the diameter of the lumen 370 of the second segment 330 of the endovascular device is at least 24.7 French (Fr). According to some embodiments, the diameter of the lumen 370 of the second segment 330 of the endovascular device is at least 24.8 French (Fr). According to some embodiments, the diameter of the lumen 370 of the second segment 330 of the endovascular device is at least 24.9 French (Fr). According to some embodiments, the diameter of the lumen 370 of the second segment 330 of the endovascular device is at least 25.0 French (Fr). According to some embodiments, the diameter of the lumen 370 of the second segment 330 of the endovascular device is at least 25.0 French (Fr). According to some embodiments, the diameter of the lumen 370 of the second segment 330 of the endovascular device is at least 25.1 French (Fr). According to some embodiments, the diameter of the lumen 370 of the second segment 330 of the endovascular device is at least 25.2 French (Fr). According to some embodiments, the diameter of the lumen 370 of the second segment 330 of the endovascular device is at least 25.3 French (Fr). According to some embodiments, the diameter of the lumen 370 of the second segment 330 of the endovascular device is at least 25.4 French (Fr). According to some embodiments, the diameter of the lumen 370 of the second segment 330 of the endovascular device is at least 25.5 French (Fr). According to some embodiments, the diameter of the lumen 370 of the second segment 330 of the endovascular device is at least 25.6 French (Fr). According to some embodiments, the diameter of the lumen 370 of the second segment 330 of the endovascular device is at least 25.7 French (Fr). According to some embodiments, the diameter of the lumen 370 of the second segment 330 of the endovascular device is at least 25.8 French (Fr). According to some embodiments, the diameter of the lumen 370 of the second segment 330 of the endovascular device is at least 25.9 French (Fr). According to some embodiments, the diameter of the lumen 370 of the second segment 330 of the endovascular device is at least 26.0 French (Fr). According to some embodiments, the diameter of the lumen 370 of the second segment 330 of the endovascular device is at least 26.1 French (Fr). According to some embodiments, the diameter of the lumen 370 of the second segment 330 of the endovascular device is at least 26.2 French (Fr). According to some embodiments, the diameter of the lumen 370 of the second segment 330 of the endovascular device is at least 26.3 French (Fr). According to some embodiments, the diameter of the lumen 370 of the second segment 330 of the endovascular device is at least 26.4 French (Fr). According to some embodiments, the diameter of the lumen 370 of the second segment 330 of the endovascular device is at least 26.5 French (Fr). According to some embodiments, the diameter of the lumen 370 of the second segment 330 of the endovascular device is at least 26.6 French (Fr). According to some embodiments, the diameter of the lumen 370 of the second segment 330 of the endovascular device is at least 26.7 French (Fr). According to some embodiments, the diameter of the lumen 370 of the second segment 330 of the endovascular device is at least 26.8 French (Fr). According to some embodiments, the diameter of the lumen 370 of the second segment 330 of the endovascular device is at least 26.9 French (Fr). According to some embodiments, the diameter of the lumen 370 of the second segment 330 of the endovascular device is at least 27.0 French (Fr). According to some embodiments, the diameter of the lumen 370 of the second segment 330 of the endovascular device is at least 27.1 French (Fr). According to some embodiments, the diameter of the lumen 370 of the second segment 330 of the endovascular device is at least 27.2 French (Fr). According to some embodiments, the diameter of the lumen 370 of the second segment 330 of the endovascular device is at least 27.3 French (Fr). According to some embodiments, the diameter of the lumen 370 of the second segment 330 of the endovascular device is at least 27.4 French (Fr). According to some embodiments, the diameter of the lumen 370 of the second segment 330 of the endovascular device is at least 27.5 French (Fr). According to some embodiments, the diameter of the lumen 370 of the second segment 330 of the endovascular device is at least 27.6 French (Fr). According to some embodiments, the diameter of the lumen 370 of the second segment 330 of the endovascular device is at least 27.7 French (Fr). According to some embodiments, the diameter of the lumen 370 of the second segment 330 of the endovascular device is at least 27.8 French (Fr). According to some embodiments, the diameter of the lumen 370 of the second segment 330 of the endovascular device is at least 27.9 French (Fr). According to some embodiments, the diameter of the lumen 370 of the second segment 330 of the endovascular device is at least 28.0 French (Fr). According to some embodiments, the diameter of the lumen 370 of the second segment 330 of the endovascular device is at least 28.1 French (Fr). According to some embodiments, the diameter of the lumen 370 of the second segment 330 of the endovascular device is at least 28.2 French (Fr). According to some embodiments, the diameter of the lumen 370 of the second segment 330 of the endovascular device is at least 28.3 French (Fr). According to some embodiments, the diameter of the lumen 370 of the second segment 330 of the endovascular device is at least 28.4 French (Fr). According to some embodiments, the diameter of the lumen 370 of the second segment 330 of the endovascular device is at least 28.5 French (Fr). According to some embodiments, the diameter of the lumen 370 of the second segment 330 of the endovascular device is at least 28.6 French (Fr). According to some embodiments, the diameter of the lumen 370 of the second segment 330 of the endovascular device is at least 28.7 French (Fr). According to some embodiments, the diameter of the lumen 370 of the second segment 330 of the endovascular device is at least 28.8 French (Fr). According to some embodiments, the diameter of the lumen 370 of the second segment 330 of the endovascular device is at least 28.9 French (Fr). According to some embodiments, the diameter of the lumen 370 of the second segment 330 of the endovascular device is at least 29.0 French (Fr). According to some embodiments, the diameter of the lumen 370 of the second segment 330 of the endovascular device is at least 29.1 French (Fr). According to some embodiments, the diameter of the lumen 370 of the second segment 330 of the endovascular device is at least 29.2 French (Fr). According to some embodiments, the diameter of the lumen 370 of the second segment 330 of the endovascular device is at least 29.3 French (Fr). According to some embodiments, the diameter of the lumen 370 of the second segment 330 of the endovascular device is at least 29.4 French (Fr). According to some embodiments, the diameter of the lumen 370 of the second segment 330 of the endovascular device is at least 29.5 French (Fr). According to some embodiments, the diameter of the lumen 370 of the second segment 330 of the endovascular device is at least 29.6 French (Fr). According to some embodiments, the diameter of the lumen 370 of the second segment 330 of the endovascular device is at least 29.6 French (Fr). According to some embodiments, the diameter of the lumen 370 of the second segment 330 of the endovascular device is at least 29.7 French (Fr). According to some embodiments, the diameter of the lumen 370 of the second segment 330 of the endovascular device is at least 29.8 French (Fr). According to some embodiments, the diameter of the lumen 370 of the second segment 330 of the endovascular device is at least 29.9 French (Fr). According to some embodiments, the diameter of the lumen 370 of the second segment 330 of the endovascular device is at least 30.0 French (Fr).

According to some embodiments, the diameter is an inner diameter (ID). According to some embodiments, the diameter is an outer diameter (OD).

According to some embodiments, the described invention can be used with an embolic agent. Embolic agents include, but are not limited to, gelatin foam, polyvinyl alcohol (PVA) particles, tris-acryl gelatin microspheres (TAGM), amplatzer vascular plugs, N-butyl-2 cyanoacrylate (NBCA), ethylene vinyl alcohol (EVOH) copolymer, calcium alginate gel, absolute alcohol, precipitating hydrophobic injectable liquid (PHIL) (Microvention Inc., CA), Onyx® liquid (EVOH copolymer in dimethyl sulfoxide (DMSO) and suspended micronized tantalum powder)(ev3 Neurovascular, Irvine CA), and coils. Non-limiting examples of coils include pushable coils, injectable coils, liquid coils, detachable coils, and hydrogel coils. The described invention also can be used to help introduce other devices, including, without limitation, Luna/Artisse (Medtronic) or Web (Microvention) devices.

According to some embodiments, the described invention can be used in an endovascular procedure in a subject suffering from an anatomical variation in a blood vessel. According to some embodiments, the blood vessel comprises an anatomical variation comprising tortuosity. According to some embodiments, the blood vessel comprises an anatomical variation comprising an acute angulation. According to some embodiments, the acute angulation is an aortic arch variation. According to some embodiments, the aortic arch variation is a bovine arch variation. According to some embodiments, the acute angulation is a vertebral artery variation. According to some embodiments, the anatomical variation is a cause/increases risk of an acute stroke.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “an”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. An endovascular device including:
a tube defining a working lumen further comprising a support tube defining a support lumen, wherein the tube comprises: a first end; and a second end comprising an opening,
wherein
the opening at the second end comprises a primary opening leading to the working lumen comprising a diameter of the primary opening and a secondary opening leading to the support lumen comprising a diameter of the secondary opening, wherein the diameter of the primary opening is greater than the diameter of the secondary opening,
the first branch, the working lumen and the primary opening form a working segment comprising the working lumen and the proximal portion of the support lumen;
the support tube distal to the primary opening comprises a support segment, which comprises a distal segment of the support lumen and the secondary opening, and
the diameter of the working segment is greater than the diameter of the support segment.

2. The endovascular device according to claim 1, wherein
(a) the support segment of a single lumen device is a conduit through which a second device is advanced into a blood vessel; or
(b) the working segment of a single lumen device is a conduit through which a second device is advanced into a blood vessel; or
(c) the support segment or the working segment of a dual lumen device is a conduit through which a second device is advanced into a blood vessel.

3. The endovascular device according to claim 2, wherein the second device is a diagnostic catheter, a therapeutic catheter, a therapeutic balloon, a retrievable balloon, a therapeutic stent, a retrievable stent, a flow-diverting stent, a coil, a wire, an endoluminal mesh, an endosaccular mesh, an embolic agent, another endovascular device or a combination thereof.

4. The endovascular device according to claim 1, wherein
(a) for intracranial applications, length of the support segment of a single lumen device, ranges from 0.01% to about 20% of the length of the working segment; or
(b) for peripheral applications,
(i) length of the support segment ranges from at least 0.01% to at least 200% of the length of the working segment; or
(ii) length of the support segment is about 0.05 cm to about 320 cm; or
(c) length of the working segment ranges from at least 20 cm to at least 160 cm.

5. The endovascular device according to claim 1, wherein
the support segment comprises one or more inflatable balloons encircling the support segment, the working segment comprises one or more inflatable balloons encircling the working segment, or a combination thereof.

6. The endovascular device according to claim 5, wherein
(a) when a single balloon on one side of the endovascular device that comprises the side-hole is effective to deflect the device, to provide more room between the side-hole and a side-wall aneurysm, or to center the endovascular device; or
(b) the two or more inflatable balloons when selectively inflated are effective to center the primary opening of the endovascular device on a base of an aneurysm in vivo; or
(c) the two or more inflatable balloons when selectively inflated are effective to center the secondary opening of the endovascular device on a base of an aneurysm in vivo.

7. The endovascular device according to claim 1, wherein the endovascular device comprises a distal inflatable balloon.

8. The endovascular device according to claim 6, wherein
(a) the selectively inflated inflatable balloons are effective to occlude blood flow; or
(b) the selectively inflated inflatable balloons are effective to reverse direction of blood flow; or
(c) the selectively inflated inflatable balloons are effective to position the catheter in a desired position within a blood vessel.

9. The endovascular device according to claim 6, further comprising a lumen positioned in association with each balloon that is adapted to fill and empty each selectively inflatable balloon.

10. The endovascular device according to claim 1, wherein
(a) the support segment comprises a second device separate from the endovascular device, wherein the second device is effective to provide added support to the endovascular device; or
(b) the support segment is in form of a geometric shape selected from the group consisting of a “C” shape, a “J” shape and an “S” shape; or
(c) the support segment is in form of a pre-shaped configuration; or
(d) the support segment is in form of an adjustable shape; or
(e) the support segment for insertion into the body of a subject is in a straight configuration and assumes a pre-formed geometric shape in situ; or
(f) the support segment comprises a shape-memory polymer (SMP); or
(g) the support segment comprises a shape-memory alloy (SMA).

11. The endovascular device according to claim 10, wherein the second device separate from the endovascular device that provides added support is a stent, a retrievable stent, a balloon, a retrievable balloon, a wire or combination thereof.

12. The endovascular device according to claim 11, wherein the second device separate from the endovascular device is adapted to be incorporated directly into the endovascular device; or is a second device adapted to be deployed through the support lumen of the endovascular device.

13. The endovascular device according to claim 1, wherein
a side-hole divides the endovascular device into a first segment and a second segment;
the first segment comprising the primary opening and the lumen,
extends from the primary opening to the side hole; and
forms the working segment comprising the primary opening, the lumen, and the side-hole; and
the second segment, which extends from the side hole to the second opening/end; forms
the support segment comprising the second opening/end and the lumen.

14. The endovascular device according to claim 1, wherein the endovascular device is an inner catheter disposed at least partially within the working lumen of an outer support catheter, wherein
(a) the inner catheter comprises a working distal side-hole that separates the working segment of the inner catheter from the distal support segment of the inner catheter;
(b) the inner catheter is connected to a set of inner catheter grips at the proximal end of the endovascular device; and the outer catheter is connected to a set of outer catheter grips, wherein the inner catheter grips and outer catheter grips are adapted for rotation of one catheter relative to the other catheter; and
(c) the rotation is effective to center the working distal side-hole of the inner catheter in proper alignment with a blood vessel origin, for support in accessing the blood vessel in proper alignment with a targeted lesion or other targeted anatomy, or a combination thereof.

15. The endovascular device according to claim 14, wherein
(a) the inner catheter is a single lumen catheter; or
(b) the inner catheter is a dual lumen catheter.

16. The endovascular device according to claim 14, wherein
(a) one or more radiopaque markers can be used to identify position of the side hole; or
(b) intravascular ultrasound (IVUS) can be incorporated into the endovascular device to help identify position of the side-hole relative to intraluminal anatomy including a neck of an aneurysm, a base of an aneurysm, an origin of a vessel branch, other targeted anatomy, or a combination thereof.

17. The endovascular device according to claim 1, wherein the primary opening comprises an extension extending from the primary opening at an angle (angled extension), wherein
(a) the angle of the angled extension is fixed or adjustable; or
(b) the angle of the angled extension ranges from an angle of at least 1 degree to an angle of at least 359 degrees; or
(c) a length of the angled extension is less than a length of the primary opening; or
(d) the primary opening is adapted to accommodate extension of the angled extension through the primary opening when in a vessel.

18. The endovascular device according to claim 1, wherein the endovascular device is
(a) an intracranial endovascular device; or
(b) a peripheral blood vessel endovascular device; or
(c) a cardiac endovascular device.

19. The endovascular device according to claim 1, wherein
the first end comprises a bifurcation, the bifurcation at the first end comprising
a first branch comprising a diameter of the first branch and a length of the first branch; and
a second branch comprising a diameter of the second branch and a length of the second branch.

20. The endovascular device according to claim 19, wherein the diameter of the first branch comprising the working lumen is greater than the diameter of the second branch comprising the support lumen.

21. A catheter comprising two or more inflatable balloons along a segment of its outer circumference that are adapted to be selectively inflated to position the catheter within a blood vessel.
